(19) United States
(12) Patent Application Publication
(10) Pub. No.: US 2024/0083934 A1
(43) Pub. Date: Mar. 14, 2024

(54) N-ACETYLGALACTOSAMINE (GALNAC)-DERIVED COMPOUNDS AND OLIGONUCLEOTIDES

(71) Applicant: ADARx Pharmaceuticals, Inc., San Diego, CA (US)

(72) Inventors: Zhen Li, San Diego, CA (US); Rui Zhu, San Diego, CA (US); Mehdi Michel Djamel Numa, San Diego, CA (US); Bo Cheng, San Diego, CA (US); Chase Robert Olsson, San Diego, CA (US); Chandramouli Chiruta, San Diego, CA (US); Indrasena Reddy Kummetha, San Diego, CA (US)

(73) Assignee: ADARx Pharmaceuticals, Inc., San Diego, CA (US)

(21) Appl. No.: 18/030,968

(22) PCT Filed: Oct. 8, 2021
(86) PCT No.: PCT/US2021/054318

§ 371 Date: Apr. 7, 2023

Related U.S. Application Data
(60) Provisional application No. 63/251,580, filed on Oct. 1, 2021.
(60) Provisional application No. 63/146,276, filed on Feb. 5, 2021.
(60) Provisional application No. 63/141,300, filed on Jan. 25, 2021.
(60) Provisional application No. 63/131,317, filed on Dec. 29, 2020.
(60) Provisional application No. 63/113,801, filed on Nov. 13, 2020.
(60) Provisional application No. 63/089,936, filed on Oct. 9, 2020.

(51) Int. Cl. C07H 19/067; C07H 19/10; C07H 21/02

(52) U.S. Cl. CPC C07H 19/067; C07H 19/10; C07H 21/02

(57) ABSTRACT

Provided herein are N-acetylgalactosamine (GalNAc)-derived compounds, modified oligonucleotides, and methods of modulating protein function and treating diseases, disorders, and symptoms in a subject.

### RELATED APPLICATIONS

This application claims priority to U.S. Provisional Application U.S. Ser. No. 63/251,580, filed Oct. 1, 2021, U.S. Provisional Application U.S. Ser. No. 63/141,300, filed Jan. 25, 2021, U.S. Provisional Application U.S. Ser. No. 63/131,317, filed Dec. 29, 2020, and U.S. Provisional Application U.S. Ser. No. 63/089,936, filed Oct. 9, 2020, as well as to U.S. Provisional Application U.S. Ser. No. 63/146,276, filed Feb. 5, 2021 and U.S. Provisional Application U.S. Ser. No. 63/113,801, filed Nov. 13, 2020, each of which is incorporated herein by reference.

### BACKGROUND

In the use of compounds in therapeutic, prophylactic, or diagnostic applications, it is often desirable that the compounds be delivered to a specific location (for example, to desired cell(s)) to enhance the therapeutic or prophylactic effect or to be advantageous for diagnostic purposes. This is frequently the case when attempting to deliver a therapeutic compound in vivo. Further, being able to efficiently deliver a compound to a specific location can limit or potentially eliminate unintended consequences (such as off-target effects) that may be caused by administration of the compound. One strategy to facilitate delivery of a compound, such as a therapeutic, prophylactic, or diagnostic compound, to a desired location in vivo, is by linking or attaching the compound to a targeting ligand.

One class of compounds that can be targeted using targeting ligands are oligomeric compounds. Oligomeric compounds that include nucleotide sequences at least partially complementary to a target nucleic acid have been shown to alter the function and activity of the target both in vitro and in vivo. When delivered to a cell containing a target nucleic acid (such as mRNA), oligomeric compounds have been shown to modulate the expression of the target resulting in altered transcription or translation of the target nucleic acid. In certain instances, the oligomeric compound can reduce the expression of the gene by inhibiting the nucleic acid target and/or triggering the degradation of the target nucleic acid.

If the target nucleic acid is mRNA, one mechanism by which an expression-inhibiting oligomeric compound can modulate the expression of the mRNA target is through RNA interference. RNA interference is a biological process by which RNA or RNA-like molecules (such as chemically modified RNA molecules) are able to silence gene expression through degradation. Additionally, single-stranded RNA and RNA-like molecules, which can also include modified nucleotides and have one or more non-phosphodiester linkages, can also alter the expression of a target nucleic acid, such as a target mRNA.

Another class of compounds that can be targeted using targeting ligands are small molecule compounds. The small molecule compounds (e.g., an organic compound having a molecular weight of ca. 1000 daltons or less) are typically shown to alter the function and/or activity of the target such that disease and/or disease symptoms are modulated or ameliorated, or are typically useful as a diagnostic marker when localized to the target. More efficient delivery of a compound to a specific location can limit or potentially eliminate unintended consequences (such as off-target effects) that may be caused by administration of the compound and provide improved localization of a diagnostic compound.

### BRIEF SUMMARY OF THE INVENTION

The invention is directed towards compounds (e.g. any of those delineated herein), modified oligonucleotides, and methods of modulating protein function and treating diseases, disorders, and symptoms in a subject. The methods can comprise the compounds and modified oligonucleotides disclosed herein.

It is understood that the embodiments of the invention discussed below with respect to the preferred variable selections can be taken alone or in combination with one or more embodiments, or preferred variable selections of the invention, as if each combination were explicitly listed herein.

In one aspect, provided are compounds of Formula (I-a), (I-b), (I-c), and (I-d), and salts, solvates, and hydrates thereof:

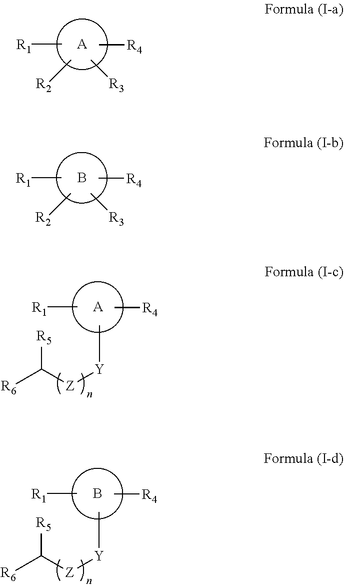

wherein:
[symbol] is Ring A, wherein each Ring A is independently optionally substituted carbocyclyl or optionally substituted heterocyclyl;
[symbol] is Ring B, wherein each Ring B is independently optionally substituted aryl or optionally substituted heteroaryl;
each n is independently 0, 1, 2, 3, or 4;
each Y is independently O, CH₂, S, S(═O), S(═O)₂, NH, substituted amino, NHC(═O), C(═O)NH, P(═O)(OH)—O—, P(═O)(SH)—O, P(═S)(SH)—O, —O—P(═O)(OH)—O—, —O—P(═O)(SH)—O—, —O—P(═S)(SH)—O—, —O—P(═O)(OH)—, —O—P(═O)(SH)—, —O—P(═S)(SH)—;
each Z is independently an optionally substituted alkyl group, an optionally substituted alkenyl group, an optionally substituted alkynyl group, or ethylene glycol;
each R₁ is independently alkyl-O-phosphoramidite, alkyl phosphoramidite, optionally substituted alkenyl phosphoramidite, or optionally substituted alkynyl phosphoramidite;
R₂ and R₅ are each independently optionally substituted alkyl-O-GalNAc, polyethylene glycol-O-GalNAc, optionally substituted alkenyl-O-GalNAc, optionally substituted alkynyl-O-GalNAc, alkyl-S(═O)-alkyl-GalNAc, alkyl-S(═O)₂-alkyl-GalNAc, alkyl-S(═O)—NH-alkyl, alkyl-S(═O)₂—NH-alkyl-GalNAc, alkyl-P P(═O)(—O—)—NH-alkyl-GalNAc, alkyl-O—P(═O)(—O—)—O-alkyl-GalNAc, alkyl-O—P(—O—)(═S)—O-alkyl-GalNAc, or alkyl-O—P(—S—)(═S)—O-alkyl-GalNAc;
R₃ and R₆ are each independently optionally substituted alkyl-O-GalNAc, optionally substituted alkenyl-O-GalNAc, optionally substituted alkynyl-O-GalNAc, alkyl-S(═O)-alkyl-GalNAc, alkyl-S(═O)₂-alkyl-GalNAc, alkyl-S(═O)—NH-alkyl, alkyl-S(═O)₂—NH-alkyl-GalNAc, alkyl-P P(═O)(—O—)—NH-alkyl-GalNAc, alkyl-O—P(═O)(—O—)—O-alkyl-GalNAc, alkyl-O—P(—O—)(═S)—O-alkyl-GalNAc, or alkyl-O—P(—S—)(═S)—O-alkyl-GalNAc; and
each R₄ is independently optionally substituted alkyl-O-GalNAc, optionally substituted alkenyl-O-GalNAc, optionally substituted alkynyl-O-GalNAc, alkyl-S(═O)-alkyl-GalNAc, alkyl-S(═O)₂-alkyl-GalNAc, alkyl-S(═O)—NH-alkyl, alkyl-S(═O)₂—NH-alkyl-GalNAc, alkyl-P P(═O)(—O—)—NH-alkyl-GalNAc, alkyl-O—P(═O)(—O—)—O-alkyl-GalNAc, alkyl-O—P(—O—)(═S)—O-alkyl-GalNAc, or alkyl-O—P(—S—)(═S)—O-alkyl-GalNAc.

In certain embodiments, the compound is of Formula (I-a).

In certain embodiments, the compound is of Formula (I-b).

In certain embodiments, the compound is of Formula (I-c).

In certain embodiments, the compound is of Formula (I-d).

In certain embodiments, R₂ and R₃ are the same.

In certain embodiments, Ring A is optionally substituted carbocyclyl. In certain embodiments, Ring A is optionally substituted heterocyclyl.

In certain embodiments, Ring B is optionally substituted aryl. In certain embodiments, Ring B is optionally substituted heteroaryl.

In another aspect, provided herein are compounds of Formula (II-a), (II-b), and (II-c), and salts, solvates, and hydrates thereof:

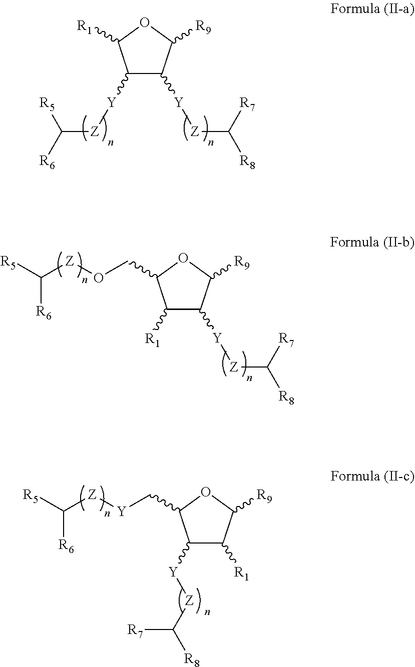

wherein:
each n is independently 0, 1, 2, 3, or 4;
each Y is independently O, CH₂, S, S(═O), S(═O)₂, NH, substituted N group, NHC(═O), C(═O)NH, P(═O)(OH)—O—, P(═O)(SH)—O, P(═S)(SH)—O, —O—P(═O)(OH)—O—, —O—P(═O)(SH)—O—, —O—P(═S)(SH)—O—, —O—P(═O)(OH)—, —O—P(═O)(SH)—, —O—P(═S)(SH)—;
each Z is independently an optionally substituted alkyl group, an optionally substituted alkenyl group, an optionally substituted alkynyl group, or ethylene glycol;
each R₁ is independently alkyl-O-phosphoramidite, optionally substituted alkenyl phosphoramidite, or optionally substituted alkynyl phosphoramidite;
R₅, R₆, R₇, and R₈ are each independently optionally substituted alkyl-O-GalNAc, polyethylene glycol-O-GalNAc, optionally substituted alkenyl-O-GalNAc, optionally substituted alkynyl-O-GalNAc, alkyl-S(═O)-alkyl-GalNAc, alkyl-S(═O)₂-alkyl-GalNAc, alkyl-S(═O)—NH-alkyl, alkyl-S(═O)₂—NH-alkyl-GalNAc, alkyl-P P(═O)(—O—)—NH-alkyl-GalNAc, alkyl-O—P(═O)(—O—)—O-alkyl-GalNAc, alkyl-O—P(—O—)(═S)—O-alkyl-GalNAc, or alkyl-O—P(—S—)(═S)—O-alkyl-GalNAc; and
R₉ is H, adenine, guanine, thymine, cytosine, uracil, inosine (I), or a nucleobase variant.

In certain embodiments, the compound is of Formula (II-a).

In certain embodiments, the compound is of Formula (II-b).

In certain embodiments, the compound is of Formula (II-c).

In certain embodiments, three of R₅, R₆, R₇, and R₈ are each independently optionally substituted alkyl-O-GalNAc, polyethylene glycol-O-GalNAc, optionally substituted alkenyl-O-GalNAc, optionally substituted alkynyl-O-GalNAc, alkyl-S(═O)-alkyl-GalNAc, alkyl-S(═O)₂-alkyl-GalNAc, alkyl-S(═O)—NH-alkyl, alkyl-S(═O)₂—NH-alkyl-GalNAc, alkyl-P P(═O)(—O—)—NH-alkyl-GalNAc, alkyl-O—P(═O)(—O—)—O-alkyl-GalNAc, alkyl-O—P(—O—)(═S)—O-alkyl-GalNAc, or alkyl-O—P(—S—)(═S)—O-alkyl-GalNAc, and the remaining one of R₅, R₆, R₇, or R₈ is independently H, alkyl, alkenyl, alkynyl, halogen, substituted amine, thiol, or amide. In certain embodiments, two of R₅, R₆, R₇, and R₈ are each the same. In certain embodiments, three of R₅, R₆, R₇, and R₈ are each the same. In certain embodiments, three of R₅, R₆, R₇, and R₈ are each the same, and the remaining one of R₅, R₆, R₇, or R₈ is independently H, alkyl, alkenyl, alkynyl, halogen, substituted amine, thiol, or amide.

In certain embodiments, R₉ is H. In certain embodiments, R₉ is adenine, guanine, thymine, cytosine, or uracil.

In another aspect, provided herein are compounds of Formula (III-a) and (III-b), and salts, solvates, and hydrates thereof:

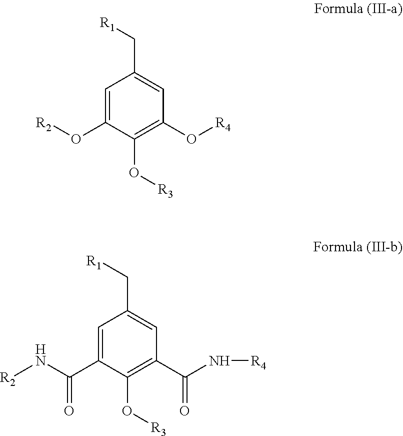

wherein:
each R₁ is independently alkyl-O-phosphoramidite, alkyl phosphoramidite, optionally substituted alkenyl phosphoramidite, or optionally substituted alkynyl phosphoramidite;
each R₂ is independently optionally substituted alkyl-O-GalNAc, polyethylene glycol-O-GalNAc, optionally substituted alkenyl-O-GalNAc, optionally substituted alkynyl-O-GalNAc, alkyl-S(═O)-alkyl-GalNAc, alkyl-S(═O)₂-alkyl-GalNAc, alkyl-S(═O)—NH-alkyl, alkyl-S(═O)₂—NH-alkyl-GalNAc, alkyl-P P(═O)(—O—)—NH-alkyl-GalNAc, alkyl-O—P(═O)(—O—)—O-alkyl-GalNAc, alkyl-O—P(—O—)(═S)—O-alkyl-GalNAc, or alkyl-O—P(—S—)(═S)—O-alkyl-GalNAc;
each R₃ is independently optionally substituted alkyl-O-GalNAc, polyethylene glycol-O-GalNAc, optionally substituted alkenyl-O-GalNAc, optionally substituted alkynyl-O-GalNAc, alkyl-S(═O)-alkyl-GalNAc, alkyl-S(═O)₂-alkyl-GalNAc, alkyl-S(═O)—NH-alkyl, alkyl-S(═O)₂—NH-alkyl-GalNAc, alkyl-P P(═O)(—O—)—NH-alkyl-GalNAc, alkyl-O—P(═O)(—O—)—O-alkyl-GalNAc, alkyl-O—P(—O—)(═S)—O-alkyl-GalNAc, or alkyl-O—P(—S—)(═S)—O-alkyl-GalNAc; and
each R₄ is independently optionally substituted alkyl-O-GalNAc, polyethylene glycol-O-GalNAc, optionally substituted alkenyl-O-GalNAc, optionally substituted alkynyl-O-GalNAc, alkyl-S(═O)-alkyl-GalNAc; alkyl-S(═O)₂-alkyl-GalNAc, alkyl-S(═O)—NH-alkyl, alkyl-S(═O)₂—NH-alkyl-GalNAc, alkyl-P P(═O)(—O—)—NH-alkyl-GalNAc, alkyl-O—P(═O)(—O—)—O-alkyl-GalNAc, alkyl-O—P(—O—)(═S)—O-alkyl-GalNAc, or alkyl-O—P(—S—)(═S)—O-alkyl-GalNAc.

In certain embodiments, the compound is of Formula (III-a).

In certain embodiments, the compound is of Formula (III-b).

In another aspect, provided herein are compounds of the formula:

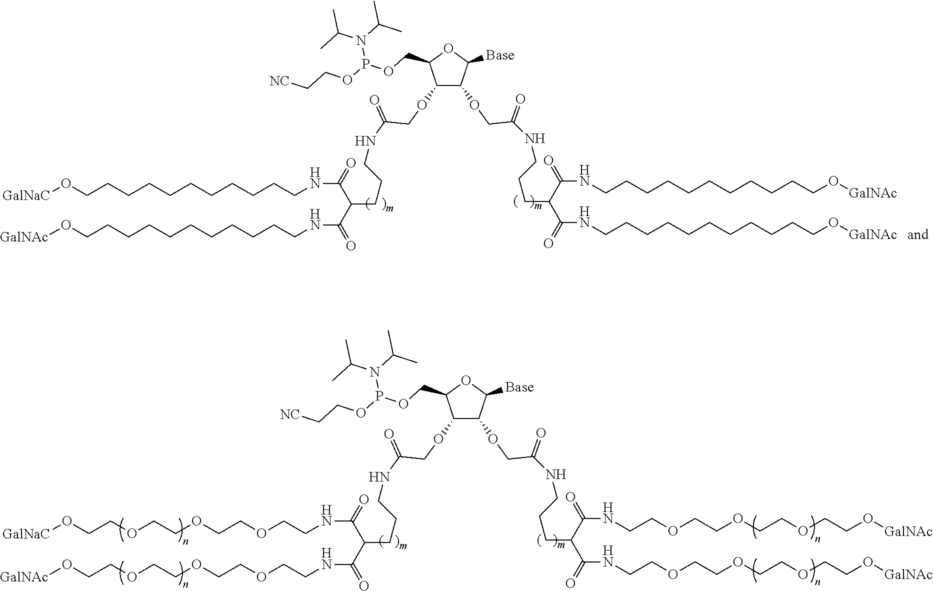

and salts, solvates, and hydrates thereof, wherein:
each n is independently an integer 0-10, inclusive;
each m is independently an integer 0-10, inclusive; and
base is adenosine, uracil, thymine, cytosine, inosine, or guanosine.

In another aspect, provided herein are compounds of Formula (IV-a), (IV-b), (IV-c), and (IV-d):

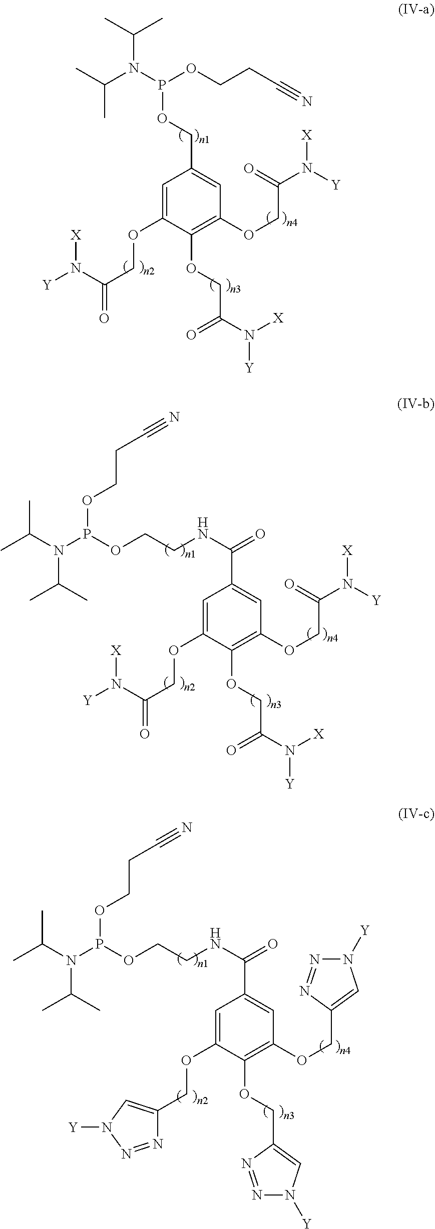

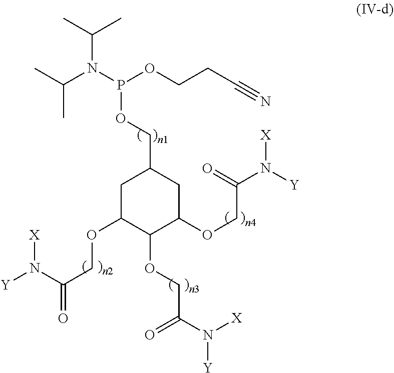

and salts, solvates, and hydrates thereof,
wherein:
n1 is independently an integer 1-10, inclusive;
n2 is independently an integer 1-10, inclusive;
n3 is independently an integer 1-10, inclusive;
n4 is independently an integer 1-10, inclusive;
each X is independently H, alkyl-GalNAc, or PEG-GalNAc; and
each Y is independently H, alkyl-GalNAc, or PEG-GalNAc.

In one aspect, provided herein are modified oligonucleotides comprising any of the compounds disclosed herein. In certain embodiments, the modified oligonucleotide comprises an siRNA, an miRNA, an ADAR recruiting molecule, an ADAR targeting molecule, a guide RNA, or an antisense nucleic acid. In some embodiments, the modified oligonucleotide comprises any of the compounds disclosed herein, wherein: (i) heterocyclyl contains only one heteroatom, which is oxygen, (ii) heterocyclyl comprises at least one heteroatom that is S; or (iii) heterocyclyl contains no nitrogen heteroatoms. In some embodiments, the modified oligonucleotide comprises any of the compounds disclosed herein, wherein Ring A is not cyclopentyl, pyrrolidinyl, piperidinyl, or morpholinyl. In some embodiments, the modified oligonucleotides comprise any of the compounds disclosed herein, wherein: (i) heteroaryl contains no nitrogen heteroatoms; (ii) heteroaryl contains only one heteroatom, which is oxygen; or (iii) heteroaryl contains only one heteroatom, which is sulfur. In some embodiments, the modified oligonucleotides comprise any of the compounds disclosed herein, wherein Ring A is not phenyl, pyridinyl, 1,3-pyrimidinyl, 1,4-pyrimidinyl, 1-quinolinyl, or 9H-purinyl.

In another aspect, provided herein are compounds of Formula (V), and salts, solvates, and hydrates thereof:

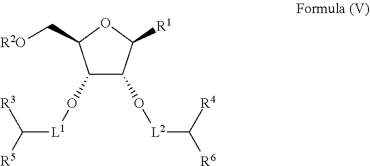

wherein:
R¹ is H, adenine, guanine, thymine, cytosine, or uracil;
R² is H, Protecting Group (PG),

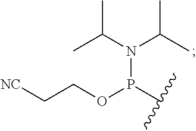 [SMILES: *P(OCCC#N)N(C(C)C)C(C)C]

L¹ is alkyl, or alkyl-C(═O)—NH-alkyl;
L² is alkyl, or alkyl-C(═O)—NH-alkyl;
R³ is H, —C═(O)—NH—(CH₂CH₂O)ⱼ-GalNAc, or —C(═O)—NH-alkyl-NH—C(═O)-alkyl-O-GalNAc;
R⁴ is H, —C═(O)—NH—(CH₂CH₂O)ₖ-GalNAc, or —C(═O)—NH-alkyl-NH—C(═O)-alkyl-O-GalNAc;
R⁵ is —C═(O)—NH—(CH₂CH₂O)ₘ-GalNAc, or —C(═O)—NH-alkyl-NH—C(═O)-alkyl-O-GalNAc;
R⁶ is —C═(O)NH—(CH₂CH₂O)ₙ-GalNAc, or —C(═O)—NH-alkyl-NH—C(═O)-alkyl-O-GalNAc;
j is an integer 1-10, inclusive;
k is an integer 1-10, inclusive;
m is an integer 1-10, inclusive; and
n is an integer 1-10, inclusive.

In another aspect, provided herein are compounds of Formula (V-a), and salts, solvates, and hydrates thereof:

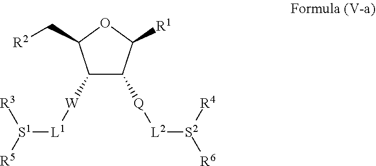

wherein:
R¹ is H, adenine, guanine, thymine, cytosine, uracil, carbocyclyl, heterocyclyl, aryl, heteroaryl, or a nucleobase isostere;
R² is H, OH, O-Protecting Group (PG),

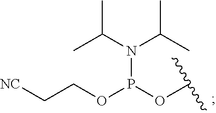 [SMILES: *OP(OCCC#N)N(C(C)C)C(C)C]

a linker, an azide, a carboxylic acid, or an amine;
L¹ is alkyl, or alkyl-C(═O)—NH-alkyl;
L² is alkyl, or alkyl-C(═O)—NH-alkyl;
R³ is H, —C═(O)—NH—(CH₂CH₂O)ⱼ-GalNAc, or —C(═O)—NH-alkyl-NH—C(═O)-alkyl-O-GalNAc;
R⁴ is H, —C═(O)—NH—(CH₂CH₂O)ₖ-GalNAc, or —C(═O)—NH-alkyl-NH—C(═O)-alkyl-O-GalNAc;
R⁵ is —C═(O)—NH—(CH₂CH₂O)ₘ-GalNAc, or —C(═O)—NH-alkyl-NH—C(═O)-alkyl-O-GalNAc;
R⁶ is —C═(O)—NH—(CH₂CH₂O)ₙ-GalNAc, or —C(═O)—NH-alkyl-NH—C(═O)-alkyl-O-GalNAc;
W and Q are each independently O, NH, CH₂, or CH₂O;
S¹ and S² are each independently C(R⁷) or N, wherein each instance of R⁷ is independently H, alkyl, heteroalkyl, or halogen;
j is an integer 1-10, inclusive;
k is an integer 1-10, inclusive;
m is an integer 1-10, inclusive; and
n is an integer 1-10, inclusive.

In another aspect, provided herein are compounds of Formula (V-b), and salts, solvates, and hydrates thereof:

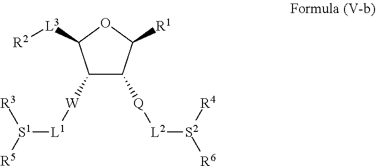

wherein:
R¹ is H, adenine, guanine, thymine, cytosine, uracil, carbocyclyl, heterocyclyl, aryl, heteroaryl, or a nucleobase isostere;
R² is an oligonucleotide;
L¹ is alkyl, or alkyl-C(═O)—NH-alkyl;
L² is alkyl, or alkyl-C(═O)—NH-alkyl;
L³ is a bond, a phosphodiester bond, a phosphorothioate bond, a triazole, a tetrazole, an amide, a reverse-amide, a carbamate, a carbonate, urea, O, S, S(═O), S(═O)₂, NH, a substituted N, alkyl, alkenyl, dienyl, alkynyl, heteroalkyl, or phosphate;
R³ is H, —C═(O)—NH—(CH₂CH₂O)ⱼ-GalNAc, or —C(═O)—NH-alkyl-NH—C(═O)-alkyl-O-GalNAc;
R⁴ is H, —C═(O)—NH—(CH₂CH₂O)ₖ-GalNAc, or —C(═O)—NH-alkyl-NH—C(═O)-alkyl-O-GalNAc;
R⁵ is —C═(O)—NH—(CH₂CH₂O)ₘ-GalNAc, or —C(═O)—NH-alkyl-NH—C(═O)-alkyl-O-GalNAc;
R⁶ is —C═(O)—NH—(CH₂CH₂O)ₙ-GalNAc, or —C(═O)—NH-allyl-NH—C(═O)-alkyl-O-GalNAc;
W and Q are each independently O, NH, CH₂, or CH₂O;
S¹ and S² are each independently C(R⁷) or N, wherein each instance of R⁷ is independently H, alkyl, heteroalkyl, or halogen;
j is an integer 1-10, inclusive;
k is an integer 1-10, inclusive;
m is an integer 1-10, inclusive; and
n is an integer 1-10, inclusive.

In certain embodiments, R³, R⁴, R⁵, and R⁶ are the same. In certain embodiments, R³, R⁵, and R⁶ are the same. In certain embodiments, R³ or R⁴ is H.

In certain embodiments, L¹ and L² are the same.

In certain embodiments, L¹ and L² are each independently alkyl; R³ is H, —C═(O)—NH—(CH₂CH₂O)ⱼ-GalNAc, or —C(═O)—NH-alkyl-NH—C(═O)-alkyl-O-GalNAc; R⁴ is H, —C═(O)—NH—(CH₂CH₂O)ₖ-GalNAc, or —C(═O)—NH-alkyl-NH—C(═O)-alkyl-O-GalNAc; R⁵ is —C═(O)—NH—(CH₂CH₂O)ₘ-GalNAc, or —C(═O)—NH-alkyl-NH—C(═O)-alkyl-O-GalNAc; and R⁶ is —C═(O)—NH—(CH₂CH₂O)ₙ-GalNAc, or —C(═O)—NH-alkyl-NH—C(═O)-alkyl-O-GalNAc.

In certain embodiments, L¹ and L² are each independently alkyl-C(═O)—NH-alkyl; R³ is H, —C═(O)—NH—(CH₂CH₂O)ⱼ-GalNAc, or —C(═O)—NH-alkyl-NH—C(═O)-alkyl-O-GalNAc; R⁴ is H, —C═(O)—NH—(CH₂CH₂O)ₖ-GalNAc, or —C(═O)—NH-alkyl-NH—C(═O)-alkyl-O-GalNAc; R⁵ is —C═(O)—NH—(CH₂CH₂O)ₘ-GalNAc, or —C(═O)—NH-alkyl-NH—C(═O)-alkyl-O-GalNAc; and R⁶ is —C═(O)—NH—(CH₂CH₂O)ₙ-GalNAc, or —C(═O)—NH-alkyl-NH—C(═O)-alkyl-O-GalNAc.

In certain embodiments, R⁴ is H.

In certain embodiments, L¹ and L² are each independently alkyl;

R³ is —C═(O)—NH—(CH₂CH₂O)ⱼ-GalNAc, or —C(═O)—NH-alkyl-NH—C(═O)-alkyl-O-GalNAc; R⁴ is H; R⁵ is —C═(O)—NH—(CH₂CH₂O)ₘ-GalNAc, or —C(═O)—NH-alkyl-NH—C(═O)-alkyl-O-GalNAc; and R⁶ is —C═(O)—NH—(CH₂CH₂O)ₙ-GalNAc, or —C(═O)—NH-alkyl-NH—C(═O)-alkyl-O-GalNAc.

In certain embodiments, L¹ and L² are each independently alkyl-C(═O)—NH-alkyl; R³ is —C═(O)—NH—(CH₂CH₂O)ⱼ-GalNAc, or —C(═O)—NH-alkyl-NH—C(═O)-alkyl-O-GalNAc; R⁴ is H; R⁵ is —C═(O)—NH—(CH₂CH₂O)ₘ-GalNAc, or —C(═O)—NH-alkyl-NH—C(═O)-alkyl-O-GalNAc; and R⁶ is —C═(O)—NH—(CH₂CH₂O)ₙ-GalNAc, or —C(═O)—NH-alkyl-NH—C(═O)-alkyl-O-GalNAc.

In certain embodiments, R³ is —C═(O)—NH—(CH₂CH₂O)ⱼ-GalNAc; R⁴ is H; R⁵ is —C═(O)—NH—(CH₂CH₂O)ₘ-GalNAc; and R⁶ is —C═(O)—NH—(CH₂CH₂O)ₙ-GalNAc.

In certain embodiments, R³ is —C(═O)—NH-alkyl-NH—C(═O)-alkyl-O-GalNAc; R⁴ is H; R⁵ is —C(═O)—NH-alkyl-NH—C(═O)-alkyl-O-GalNAc; and R⁶ is —C(═O)—NH-allyl-NH—C(═O)-allyl-O-GalNAc.

In certain embodiments, j, m, and n are each independently an integer 4-10, inclusive. In certain embodiments, j, m, and n are each the same integer 4-10, inclusive.

In certain embodiments, R¹ is H. In certain embodiments, R¹ is adenine, guanine, thymine, cytosine, or uracil. In certain embodiments R¹ is adenine. In certain embodiments, R¹ is guanine. In certain embodiments, R¹ is thymine. In certain embodiments, R¹ is cytosine. In certain embodiments, R¹ is uracil.

In certain embodiments, R² is H. In certain embodiments, R² is Protecting Group (PG). In certain embodiments, PG is an oxygen protecting group. In certain embodiments, R² is

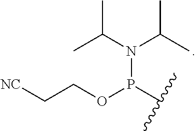 [SMILES: *P(OCCC#N)N(C(C)C)C(C)C]

In certain embodiments, any of the compounds disclosed herein, or salts, solvates, or hydrates thereof, may be used as a reagent in a chemical reaction.

In another aspect, provided herein are compounds of Formula (VI), and salts, solvates, and hydrates thereof:

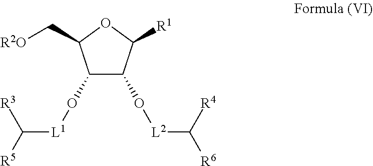

wherein:
R¹ is H, adenine, guanine, thymine, cytosine, or uracil;
R² is an oligonucleotide sequence;
L¹ is alkyl, or alkyl-C(═O)—NH-alkyl;
L² is alkyl, or alkyl-C(═O)—NH-alkyl;
R³ is H, —C═(O)—NH—(CH₂CH₂O)ⱼ-GalNAc, or —C(═O)—NH-alkyl-NH—C(═O)-alkyl-O-GalNAc;
R⁴ is H, —C═(O)—NH—(CH₂CH₂O)ₖ-GalNAc, or —C(═O)—NH-alkyl-NH—C(═O)-alkyl-O-GalNAc;
R⁵ is —C═(O)—NH—(CH₂CH₂O)ₘ-GalNAc, or —C(═O)—NH-alkyl-NH—C(═O)-alkyl-O-GalNAc;
R⁶ is —C═(O)—NH—(CH₂CH₂O)ₙ-GalNAc, or —C(═O)—NH-alkyl-NH—C(═O)-alkyl-O-GalNAc;
j is an integer 1-10, inclusive;
k is an integer 1-10, inclusive;
m is an integer 1-10, inclusive; and
n is an integer 1-10, inclusive.

In another aspect, provided herein are modified oligonucleotides comprising a compound of Formula VI. In certain embodiments, the modified oligonucleotide comprises an siRNA, an miRNA, an ADAR recruiting molecule, an ADAR targeting molecule, a guide RNA, or an antisense nucleic acid.

In one aspect, provided herein are oligonucleotides comprising a moiety of Formula (VII), or a salt, solvate, or hydrate thereof:

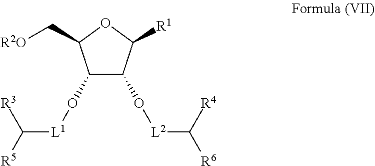

wherein:
R¹ is H, adenine, guanine, thymine, cytosine, or uracil;
R² is a bond;
L¹ is alkyl, or alkyl-C(═O)—NH-alkyl;
L² is alkyl, or alkyl-C(═O)—NH-alkyl;
R³ is H, —C═(O)—NH—(CH₂CH₂O)ⱼ-GalNAc, or —C(═O)—NH-alkyl-NH—C(═O)-alkyl-O-GalNAc;
R⁴ is H, —C═(O)—NH—(CH₂CH₂O)ₖ-GalNAc, or —C(═O)—NH-alkyl-NH—C(═O)-alkyl-O-GalNAc;
R⁵ is —C═(O)—NH—(CH₂CH₂O)ₘ-GalNAc, or —C(═O)—NH-alkyl-NH—C(═O)-alkyl-O-GalNAc;
R⁶ is —C═(O)—NH—(CH₂CH₂O)ₙ-GalNAc, or —C(═O)—NH-alkyl-NH—C(═O)-alkyl-O-GalNAc;
j is an integer 1-10, inclusive;
k is an integer 1-10, inclusive;
m is an integer 1-10, inclusive; and
n is an integer 1-10, inclusive.

In certain embodiments, the oligonucleotide comprises an siRNA, an miRNA, an ADAR recruiting molecule, an ADAR targeting molecule, a guide RNA, or an antisense nucleic acid.

In one aspect, provided herein are compositions comprising any of the compounds disclosed herein, and a pharmaceutically acceptable carrier.

In another aspect, provided herein are methods for modulating protein function in a subject, comprising administration of any of the compounds disclosed herein to the subject.

In another aspect, provided herein are methods for treating or ameliorating a disease, disorder, or symptom thereof in a subject, comprising administration of any of the compounds disclosed herein to the subject.

In another aspect, provided herein are compounds of Formula (I-a), (I-b), (I-c), and (I-d), and salts, solvates, and hydrates thereof:

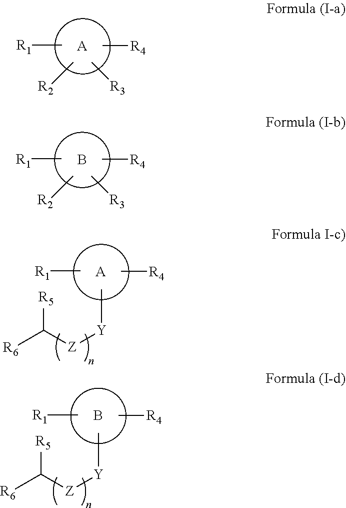

wherein:
[symbol] is Ring A, wherein each Ring A is independently optionally substituted carbocyclyl or optionally substituted heterocyclyl;
[symbol] is Ring B, wherein each Ring B is independently optionally substituted heteroaryl;
each n is independently 0, 1, 2, 3, or 4;
each Y is independently O, CH₂, S, S(═O), S(═O)₂, NH, substituted amino, NHC(═O), C(═O)NH, P(═O)(OH)—O—, P(═O)(SH)—O, P(═S)(SH)—O, —O—P(═O)(OH)—O—, —O—P(═O)(SH)—O—, —O—P(═S)(SH)—O—, —O—P(═O)(OH)—, —O—P(═O)(SH)—, —O—P(═S)(SH)—;
each Z is independently an optionally substituted alkyl group, an optionally substituted alkenyl group, an optionally substituted alkynyl group, or ethylene glycol;
each R₁ is independently alkyl-O-phosphoramidite, alkyl phosphoramidite, optionally substituted alkenyl phosphoramidite, or optionally substituted alkynyl phosphoramidite;
R₂ and R₅ are each independently optionally substituted alkyl-O-GalNAc, polyethylene glycol-O-GalNAc, optionally substituted alkenyl-O-GalNAc, optionally substituted alkynyl-O-GalNAc, alkyl-S(═O)-alkyl-GalNAc, alkyl-S(═O)₂-alkyl-GalNAc, alkyl-S(═O)—NH-alkyl, alkyl-S(═O)₂—NH-alkyl-GalNAc, alkyl-P P(═O)(—O—)—NH-alkyl-GalNAc, alkyl-O—P(═O)(—O—)—O-alkyl-GalNAc, alkyl-O—P(—O—)(═S)—O-alkyl-GalNAc, or alkyl-O—P(—S—)(═S)—O-alkyl-GalNAc;
R₃ and R₆ are each independently optionally substituted alkyl-O-GalNAc, optionally substituted alkenyl-O-GalNAc, optionally substituted alkynyl-O-GalNAc, alkyl-S(═O)-alkyl-GalNAc, alkyl-S(═O)₂-alkyl-GalNAc, alkyl-S(═O)—NH-alkyl, alkyl-S(═O)₂—NH-alkyl-GalNAc, alkyl-P P(═O)(—O—)—NH-alkyl-GalNAc, alkyl-O—P(═O)(—O—)—O-alkyl-GalNAc, alkyl-O—P(—O—)(═S)—O-alkyl-GalNAc, or alkyl-O—P(—S—)(═S)—O-alkyl-GalNAc; and
each R₄ is independently optionally substituted alkyl-O-GalNAc, optionally substituted alkenyl-O-GalNAc, optionally substituted alkynyl-O-GalNAc, alkyl-S(═O)-alkyl-GalNAc, alkyl-S(═O)₂-alkyl-GalNAc, alkyl-S(═O)—NH-alkyl, alkyl-S(═O)₂—NH-alkyl-GalNAc, alkyl-P P(═O)(—O—)—NH-alkyl-GalNAc, alkyl-O—P(═O)(—O—)—O-alkyl-GalNAc, alkyl-O—P(—O—)(═S)—O-alkyl-GalNAc, or alkyl-O—P(—S—)(═S)—O-alkyl-GalNAc.

In another aspect, provided herein are compounds of Formula (IV-a) and (IV-d):

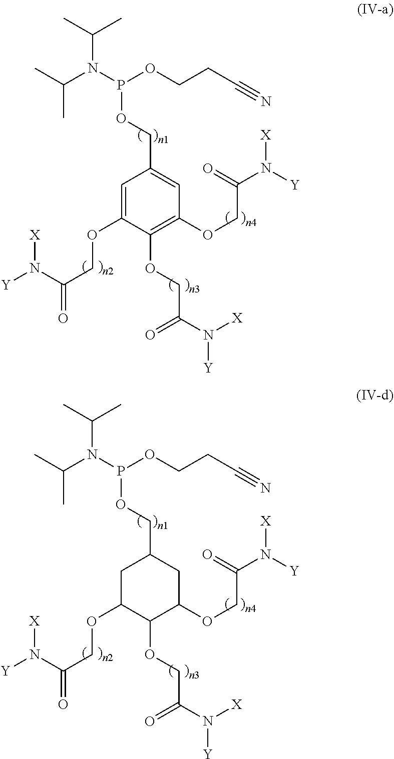

and salts, solvates, and hydrates thereof,
wherein:
n1 is independently an integer 1-10, inclusive;
n2 is independently an integer 1-10, inclusive;
n3 is independently an integer 1-10, inclusive;
n4 is independently an integer 1-10, inclusive;
each X is independently H, alkyl-GalNAc, or PEG-GalNAc; and
each Y is independently H, alkyl-GalNAc, or PEG-GalNAc.

In another aspect, compounds of Formula (VIII), and salts, solvates, and hydrates thereof, are provided:

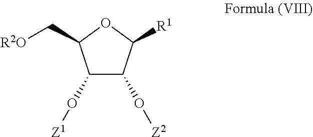

wherein:
R¹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, or optionally substituted heteroaryl;
R² is H, Protecting Group (PG),

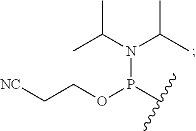 [SMILES: *P(OCCC#N)N(C(C)C)C(C)C]

Z¹ is

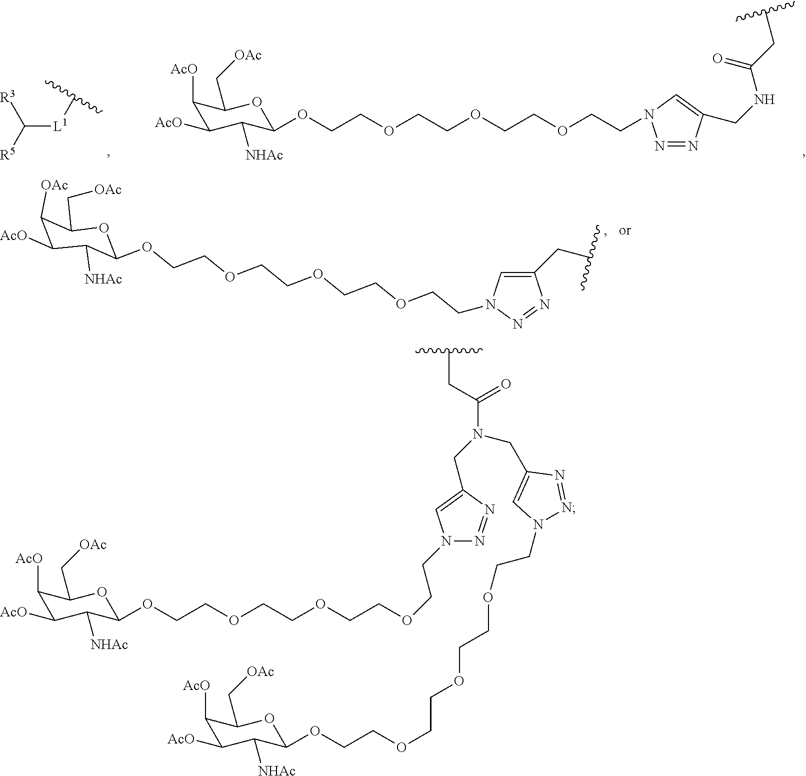

Z² is

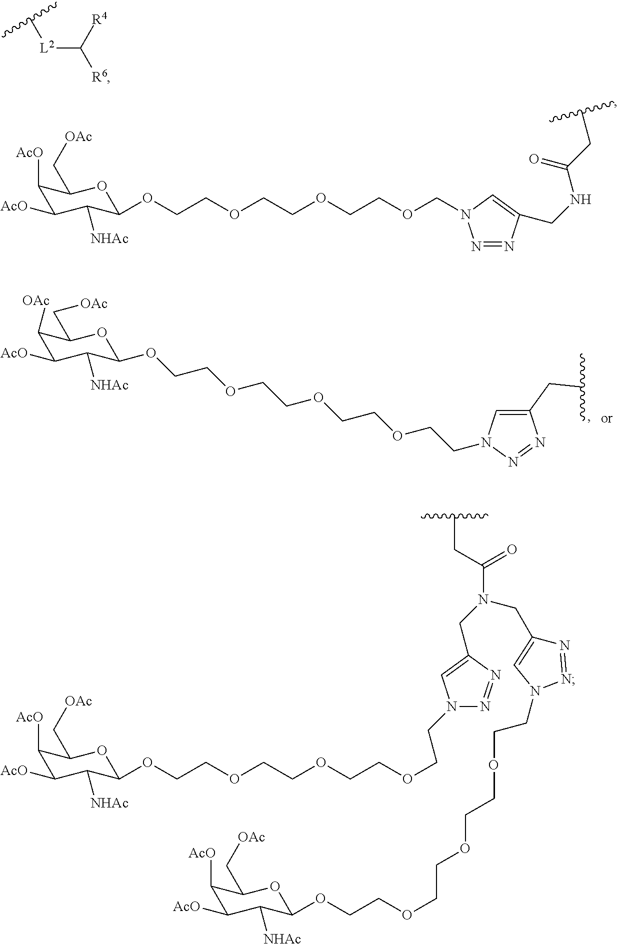

L¹ is alkyl, or alkyl-C(═O)—NH-alkyl;
L² is alkyl, or alkyl-C(═O)—NH-alkyl;
R³ is H, —C═(O)—NH—(CH₂CH₂O)ⱼ-GalNAc, or —C(═O)—NH-alkyl-NH—C(═O)-alkyl-O-GalNAc;
R⁴ is H, —C═(O)—NH—(CH₂CH₂O)ₖ-GalNAc, or —C(═O)—NH-alkyl-NH—C(═O)-alkyl-O-GalNAc;
R⁵ is —C═(O)—NH—(CH₂CH₂O)ₘ-GalNAc, or —C(═O)—NH-alkyl-NH—C(═O)-alkyl-O-GalNAc;
R⁶ is —C═(O)—NH—(CH₂CH₂O)ₙ-GalNAc, or —C(═O)—NH-alkyl-NH—C(═O)-alkyl-O-GalNAc;
j is an integer 1-10, inclusive;
k is an integer 1-10, inclusive;
m is an integer 1-10, inclusive; and
n is an integer 1-10, inclusive.

In another aspect, compounds of Formula (VIII-a), and salts, solvates, and hydrates thereof, are provided:

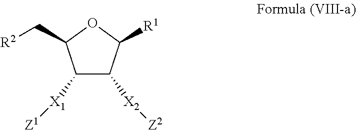

wherein:
R¹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, or optionally substituted heteroaryl, optionally substituted carbocyclyl, optionally substituted heterocyclyl, or a nucleobase isostere;
R² is H, OH, O-Protecting Group (PG),

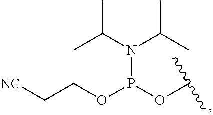 [SMILES: *OP(OCCC#N)N(C(C)C)C(C)C]

a linker, an azide, a carboxylic acid, an amine, or phosphate;
X₁ is O, optionally substituted NH, CH₂, or CH₂O;
Z¹ is

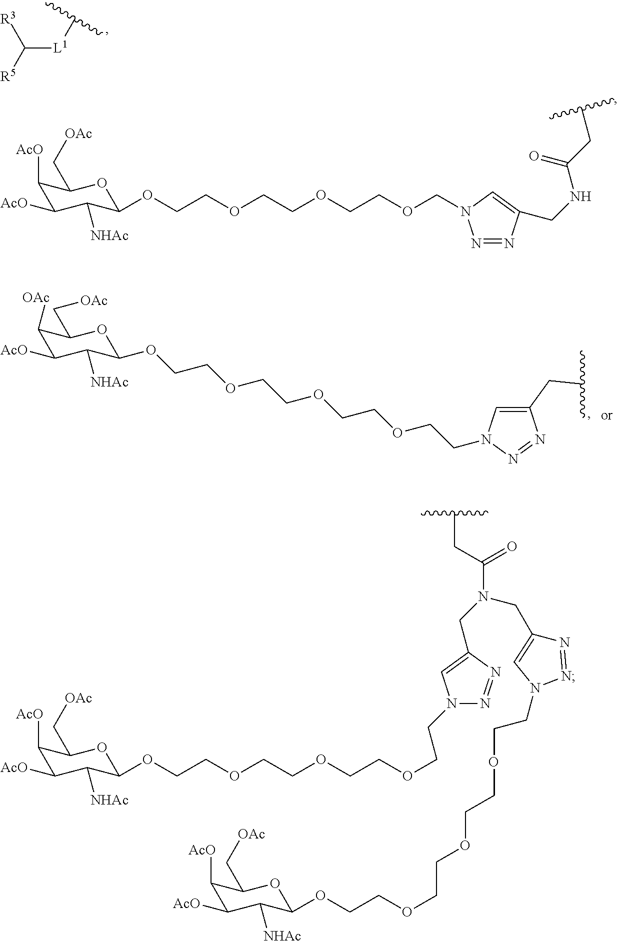

Z² is

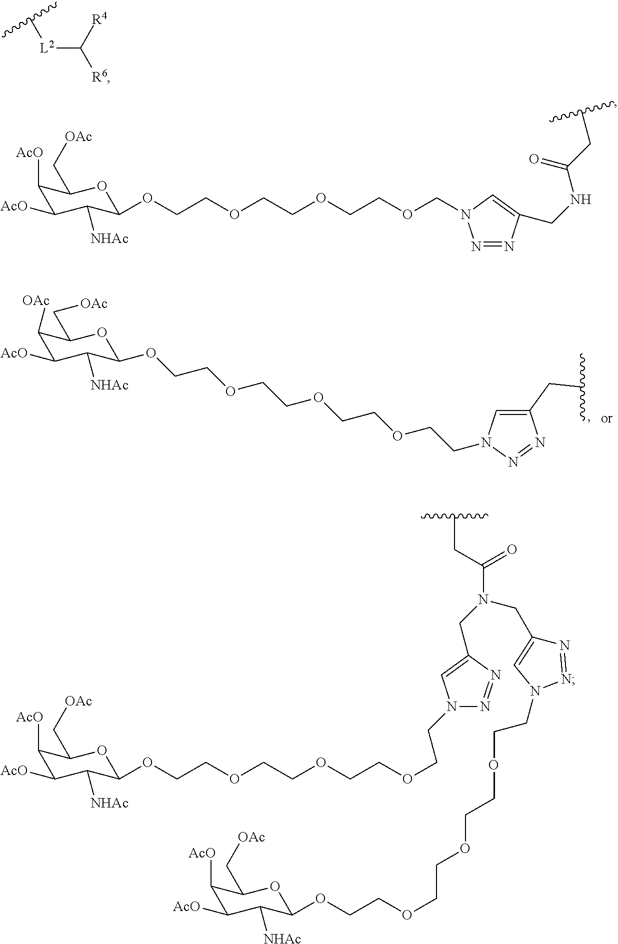

L¹ is alkyl, or alkyl-C(═O)—NH-alkyl;
L² is alkyl, or alkyl-C(═O)—NH-alkyl;
R³ is H, —C═(O)—NH—(CH₂CH₂O)ⱼ-GalNAc, or —C(═O)—NH-alkyl-NH—C(═O)-alkyl-O-GalNAc;
R⁴ is H, —C═(O)—NH—(CH₂CH₂O)ₖ-GalNAc, or —C(═O)—NH-alkyl-NH—C(═O)-alkyl-O-GalNAc;
R⁵ is —C═(O)—NH—(CH₂CH₂O)ₘ-GalNAc, or —C(═O)—NH-alkyl-NH—C(═O)-alkyl-O-GalNAc;
R⁶ is —C═(O)—NH—(CH₂CH₂O)ₙ-GalNAc, or —C(═O)—NH-alkyl-NH—C(═O)-alkyl-O-GalNAc;
j is an integer 1-10, inclusive;
k is an integer 1-10, inclusive;
m is an integer 1-10, inclusive; and
n is an integer 1-10, inclusive.

In another aspect, compounds of Formula (VIII-b), and salts, solvates, and hydrates thereof, are provided:

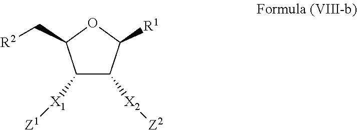

wherein:
R¹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, or optionally substituted heteroaryl, optionally substituted carbocyclyl, optionally substituted heterocyclyl, or a nucleobase isostere;
R² is H, OH, O-Protecting Group (PG),

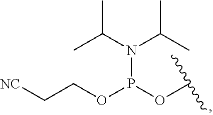 [SMILES: *OP(OCCC#N)N(C(C)C)C(C)C]

a linker, an azide, a carboxylic acid, an amine, or phosphate;
X₁ is O, optionally substituted NH, CH₂, or CH₂O;
X₂ is O, optionally substituted NH, CH₂, or CH₂O;
Z¹ is

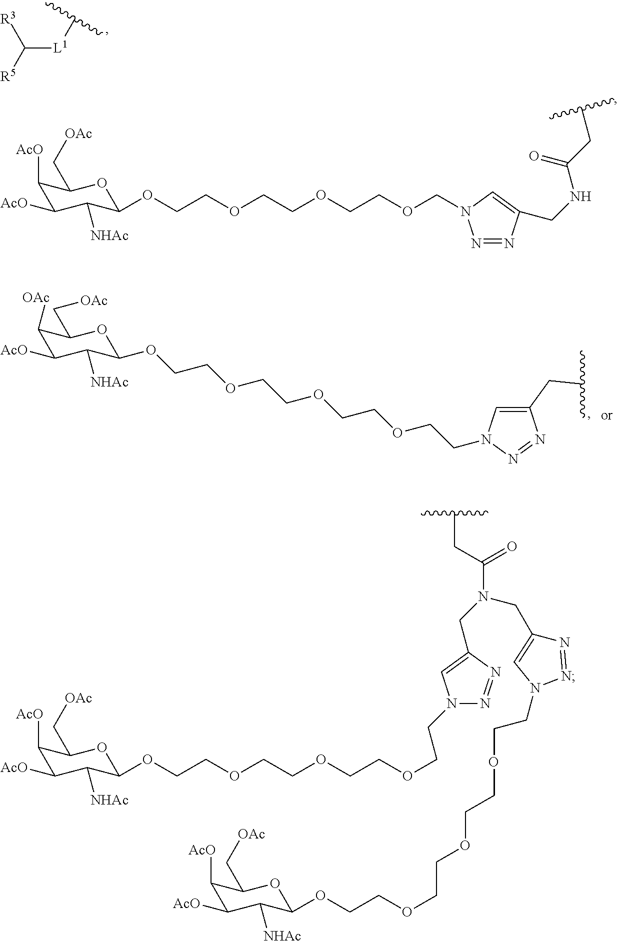

Z² is

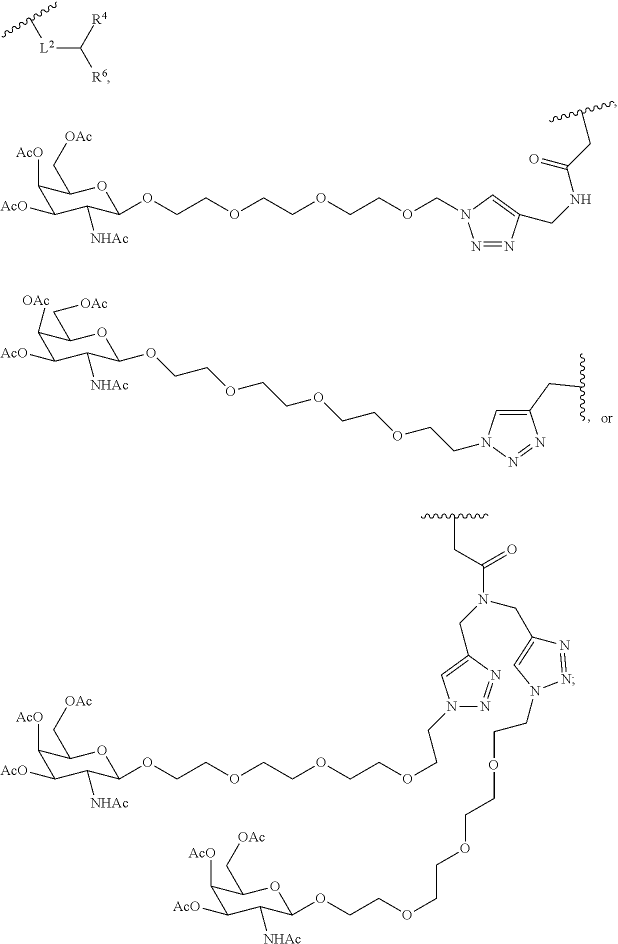

L¹ is alkyl, or alkyl-C(═O)—NH-alkyl;
L² is alkyl, or alkyl-C(═O)—NH-alkyl;
R³ is H, —C═(O)—NH—(CH₂CH₂O)ⱼ-GalNAc, or —C(═O)—NH-alkyl-NH—C(═O)-alkyl-O-GalNAc;
R⁴ is H, —C═(O)—NH—(CH₂CH₂O)ₖ-GalNAc, or —C(═O)—NH-alkyl-NH—C(═O)-alkyl-O-GalNAc;
R⁵ is —C═(O)—NH—(CH₂CH₂O)ₘ-GalNAc, or —C(═O)—NH-alkyl-NH—C(═O)-alkyl-O-GalNAc;
R⁶ is —C═(O)—NH—(CH₂CH₂O)ₙ-GalNAc, or —C(═O)—NH-alkyl-NH—C(═O)-alkyl-O-GalNAc;
j is an integer 1-10, inclusive;
k is an integer 1-10, inclusive;
m is an integer 1-10, inclusive; and
n is an integer 1-10, inclusive.

In another aspect, compounds of Formula (IX), and salts, solvates, and hydrates thereof, are provided:

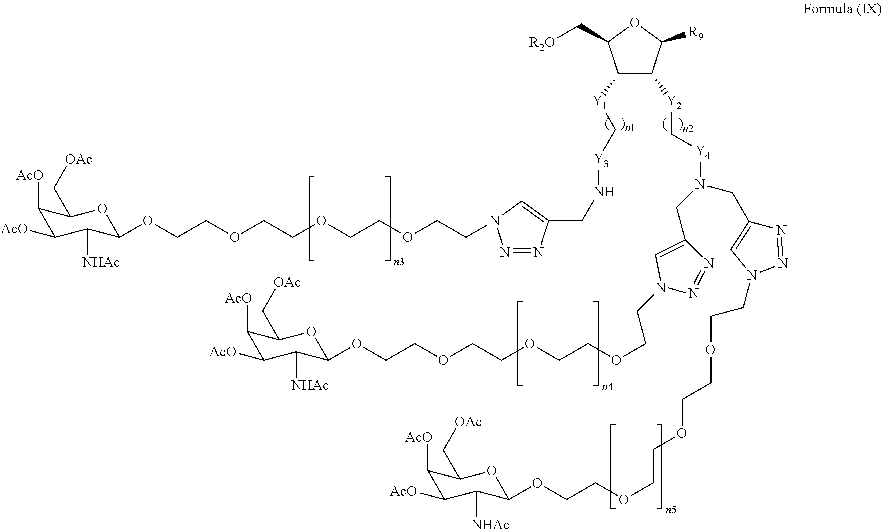

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, or optionally substituted heteroaryl;
R² is H, Protecting Group (PG), or

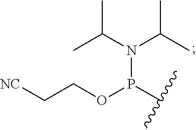 [SMILES: *P(OCCC#N)N(C(C)C)C(C)C]

Y₁ is O;
Y₂ is O;
Y₃ is CO, SO₂, P(O)O, CH₂—O—C(O), CH₂—NH—C(O), CH₂—NH—SO₂, or CH₂;
Y₄ is CO, SO₂, P(O)O, CH₂—O—C(O), CH₂—NH—C(O), CH₂—NH—SO₂, or CH₂;
n₂ is 1, 2, 3, 4, 5, or 6;
each n₁, n₃, n₄ and n₅ is independently 0, 1, 2, 3, 4, 5, or 6.

In some embodiments, the compound is of Formula (IX-a), or a salt, solvate, or hydrate thereof:

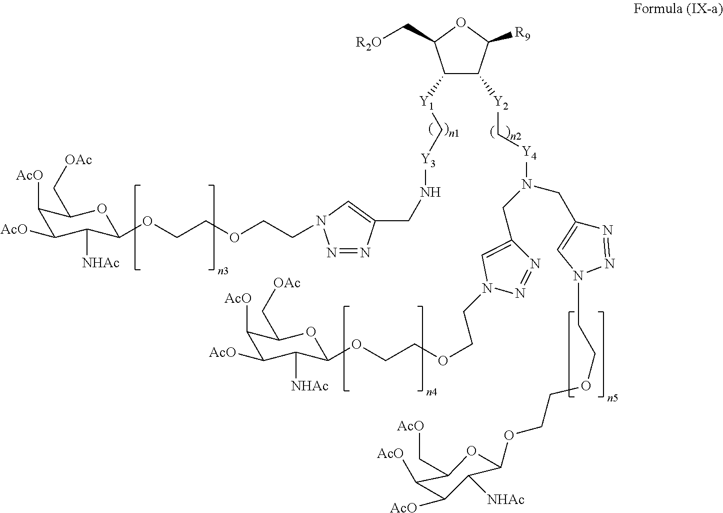

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, optionally substituted heteroaryl, optionally substituted carbocyclyl, optionally substituted heterocyclyl, or a nucleobase isostere;
R² is H, OH, O-Protecting Group (PG),

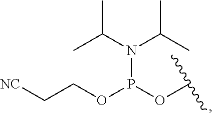 [SMILES: *OP(OCCC#N)N(C(C)C)C(C)C]

a linker, an azide, a carboxylic acid, or an amine;
Y₁ is O, optionally substituted NH, CH₂, or CH₂O;
Y₂ is O, optionally substituted NH, CH₂, or CH₂O;
Y₃ is CO, SO₂, P(O)O, CH₂—O—C(O), CH₂—NH—C(O), CH₂—NH—SO₂, or CH₂;
Y₄ is CO, SO₂, P(O)O, CH₂—O—C(O), CH₂—NH—C(O), CH₂—NH—SO₂, or CH₂;
n₂ is 0, 1, 2, 3, 4, 5, or 6;
each n₁, n₃, n₄ and n₅ is independently 0, 1, 2, 3, 4, 5, 6, 7, 8, 9, or 10.

In another aspect, compounds of Formula (X), and salts, solvates, and hydrates thereof, are provided:

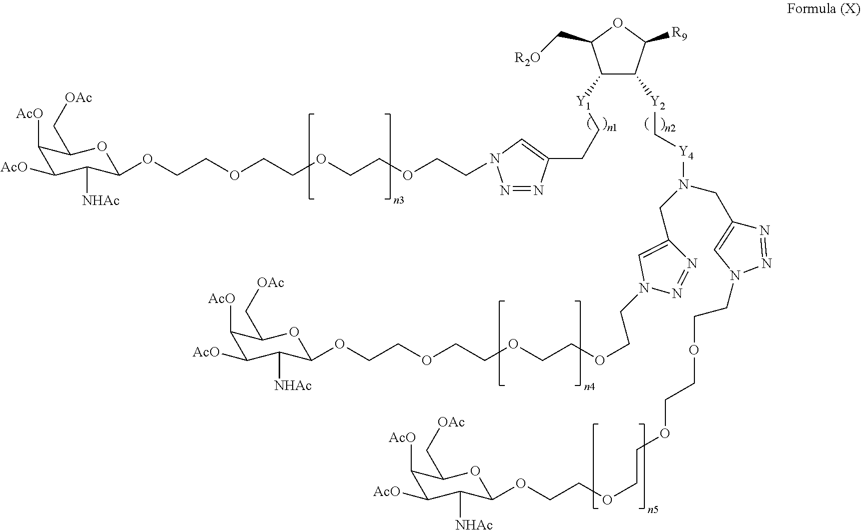

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, or optionally substituted heteroaryl;
R² is H, Protecting Group (PG), or

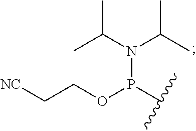 [SMILES: *P(OCCC#N)N(C(C)C)C(C)C]

Y₁ is O;
Y₂ is O;
Y₄ is CO, SO₂, P(O)O, CH₂—O—C(O), CH₂—NH—C(O), CH₂—NH—SO₂, or CH₂;
n₂ is 1, 2, 3, 4, 5, or 6;
each n₁, n₃, n₄ and n₅ is independently 0, 1, 2, 3, 4, 5, or 6.

In some embodiments, the compound is of Formula (X-a), or a salt, solvate, or hydrate thereof:

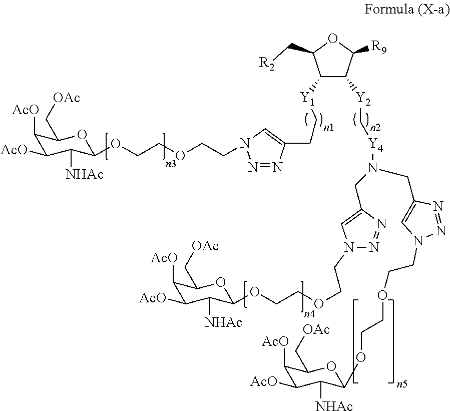

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, optionally substituted heteroaryl, optionally substituted carbocyclyl, optionally substituted heterocyclyl, or a nucleobase isostere;
R² is H, OH, O-Protecting Group (PG),

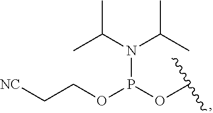 [SMILES: *OP(OCCC#N)N(C(C)C)C(C)C]

a linker, an azide, a carboxylic acid, or an amine;
Y₁ is O, optionally substituted NH, CH₂, or CH₂O;
Y₂ is O, optionally substituted NH, CH₂, or CH₂O;
Y₄ is CO, SO₂, P(O)O, CH₂—O—C(O), CH₂—NH—C(O), CH₂—NH—SO₂, or CH₂;
n₂ is 0, 1, 2, 3, 4, 5, or 6;
each n₁, n₃, n₄, and n₅ is independently 0, 1, 2, 3, 4, 5, 6, 7, 8, 9, or 10.

In some embodiments, R⁹ is uracil. In some embodiments, R⁹ is uracil comprising a protecting group. In certain embodiments, R⁹ is uracil comprising a benzoyl protecting group. In some embodiments, R⁹ is cytosine. In some embodiments, R⁹ is cytosine comprising a protecting group. In certain embodiments, R⁹ is cytosine comprising an acyl protecting group. In certain embodiments, R⁹ is cytosine comprising a benzoyl protecting group. In some embodiments, R⁹ is adenine. In some embodiments, R⁹ is adenine comprising a protecting group. In certain embodiments, R⁹ is adenine comprising a benzoyl protecting group. In some embodiments, R⁹ is guanine. In some embodiments, R⁹ is guanine comprising a protecting group. In some embodiments, R⁹ is guanine comprising a dimethylacetate protecting group.

In some embodiments, R² is O-Protecting Group (PG). In certain embodiments, R² is

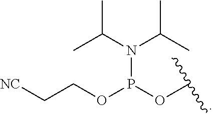 [SMILES: *OP(OCCC#N)N(C(C)C)C(C)C]

In some embodiments, Y₁ is O. In some embodiments, Y₂ is O. In certain embodiments, both Y₁ and Y₂ are O. In some embodiments, Y₃ is CO. In some embodiments, Y₄ is CO. In certain embodiments, both Y₃ and Y₄ are CO. In certain embodiments, Y₁ and Y₂ are O, and Y₃ and Y₄ are CO.

In some embodiments, n₃ is 1. In some embodiments, n₃ is 3. In some embodiments, n₃ is 5. In some embodiments, n₃ is 7. In some embodiments, n₄ is 1. In some embodiments, n₄ is 3. In some embodiments, n₄ is 5. In some embodiments, n₄ is 7. In some embodiments, n₅ is 1. In some embodiments, n₅ is 3. In some embodiments, n₅ is 5. In some embodiments, n₅ is 7. In certain embodiments, two of n₃, n₄, and n₅ are 1. In certain embodiments, all three of n₃, n₄, and n₅ are 1.

In some embodiments, the compound is of Formula (IX-b):

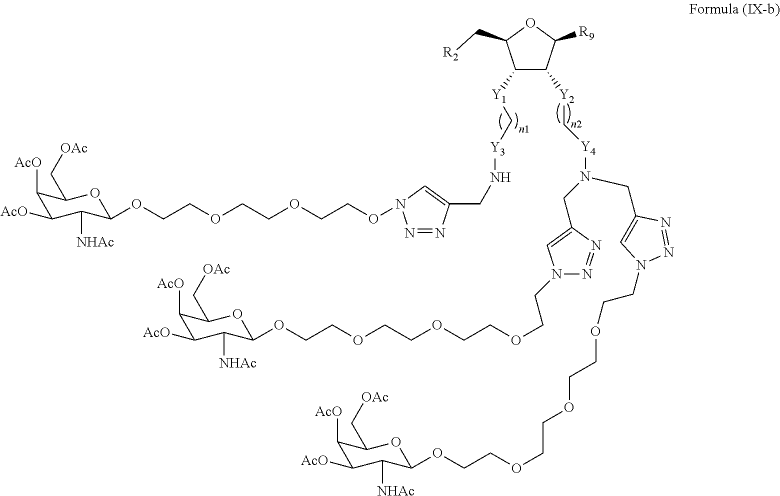

wherein R₉, R₂, Y₁, Y₂, Y₃, Y₄, n₁, and n₂ are as defined herein.

In some embodiments, the compound is of Formula (X-b):

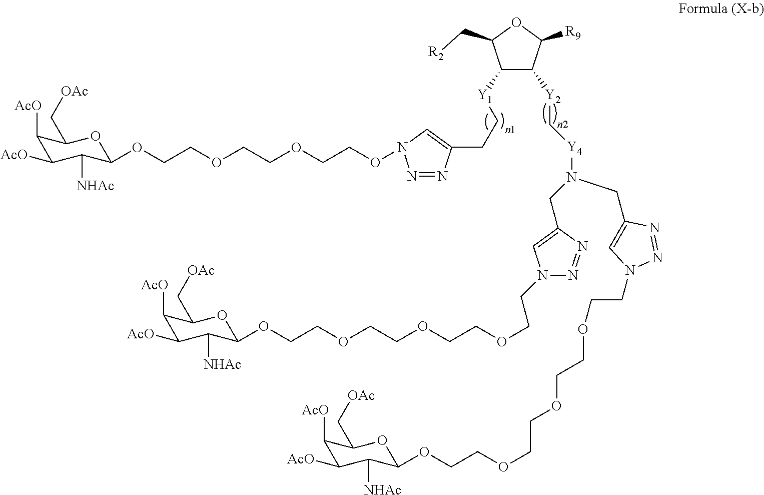

wherein R₉, R₂, Y₁, Y₂, n₁, and n₂ are as defined herein.

In some embodiments, the compound is of the Formula:

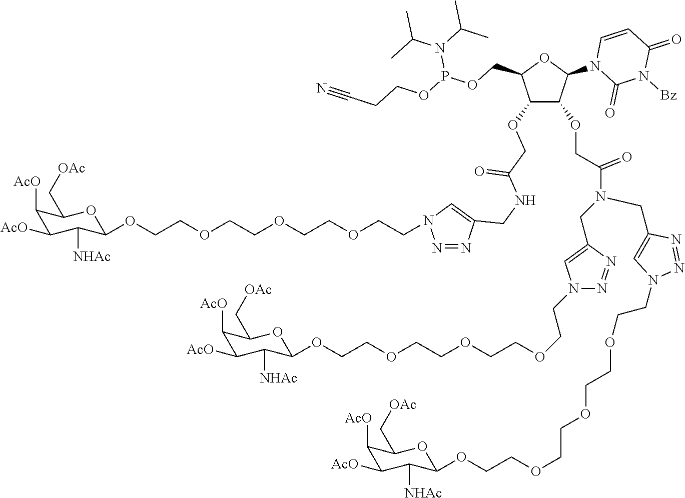

or a salt, solvate, or hydrate thereof. In some embodiments, the compound is of the Formula:

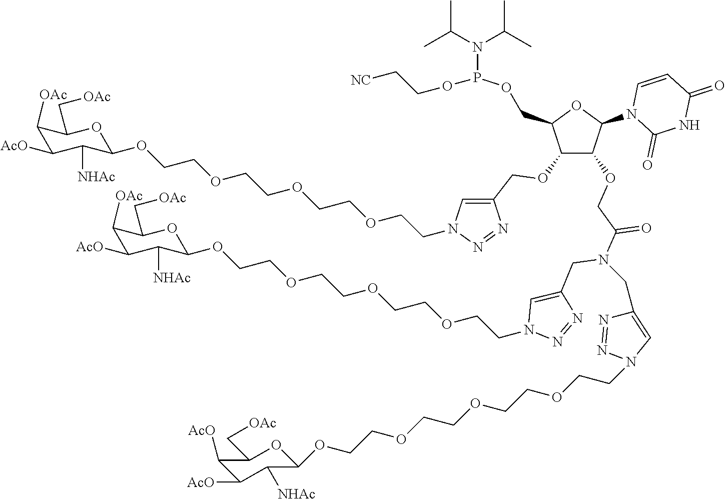 [SMILES: CC(=O)NC1C(OCCOCCOCCOCCn2cc(CO[C@H]3[C@@H](OCC(=O)N(Cc4cn(CCOCCOCCOCCOC5OC(COC(C)=O)C(OC(C)=O)C(OC(C)=O)C5NC(C)=O)nn4)Cc4cn(CCOCCOCCOCCOC5OC(COC(C)=O)C(OC(C)=O)C(OC(C)=O)C5NC(C)=O)nn4)[C@H](n4ccc(=O)[nH]c4=O)O[C@@H]3COP(OCCC#N)N(C(C)C)C(C)C)nn2)OC(COC(C)=O)C(OC(C)=O)C1OC(C)=O]

or a salt, solvate, or hydrate thereof. In some embodiments, the compound is of the Formula:

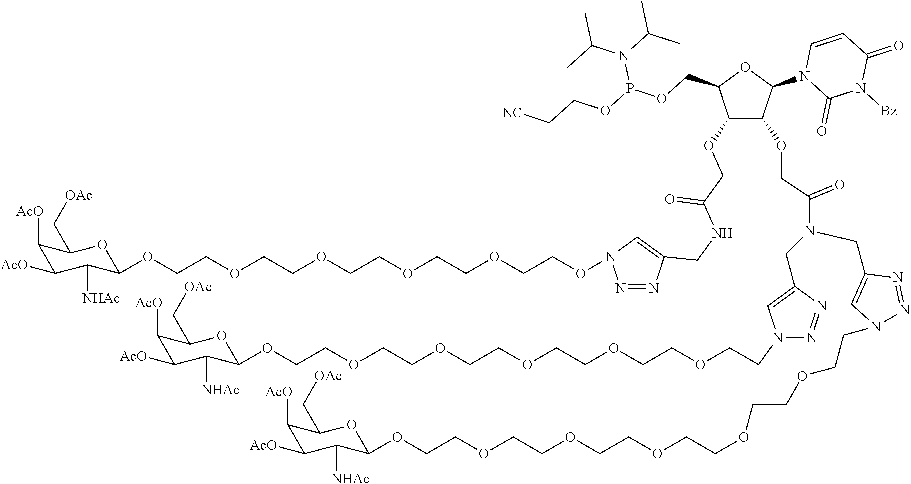

or a salt, solvate, or hydrate thereof. In some embodiments, the compound is of the Formula:

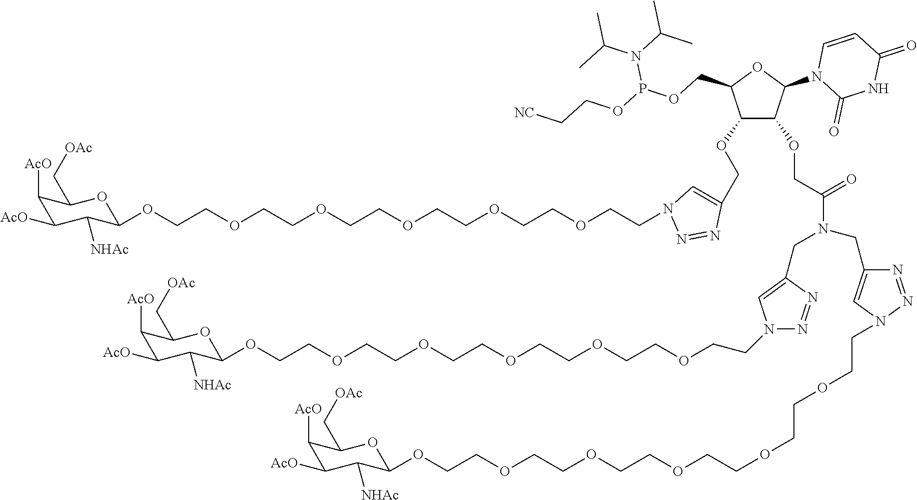 [SMILES: CC(=O)NC1C(OCCOCCOCCOCCOCCOCCn2cc(CO[C@H]3[C@@H](OCC(=O)N(Cc4cn(CCOCCOCCOCCOCCOCCOC5OC(COC(C)=O)C(OC(C)=O)C(OC(C)=O)C5NC(C)=O)nn4)Cc4cn(CCOCCOCCOCCOCCOCCOC5OC(COC(C)=O)C(OC(C)=O)C(OC(C)=O)C5NC(C)=O)nn4)[C@H](n4ccc(=O)[nH]c4=O)O[C@@H]3COP(OCCC#N)N(C(C)C)C(C)C)nn2)OC(COC(C)=O)C(OC(C)=O)C1OC(C)=O]

or a salt, solvate, or hydrate thereof. In some embodiments, the compound is of the Formula:

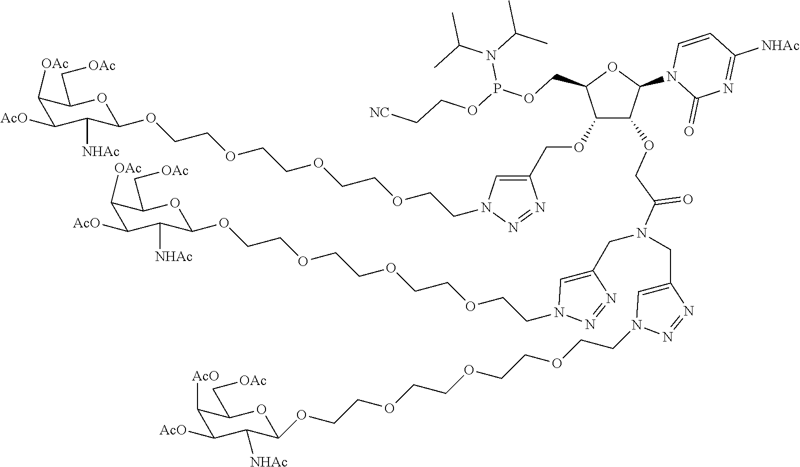 [SMILES: CC(=O)Nc1ccn([C@@H]2O[C@H](COP(OCCC#N)N(C(C)C)C(C)C)[C@@H](OCc3cn(CCOCCOCCOCCOC4OC(COC(C)=O)C(OC(C)=O)C(OC(C)=O)C4NC(C)=O)nn3)[C@H]2OCC(=O)N(Cc2cn(CCOCCOCCOCCOC3OC(COC(C)=O)C(OC(C)=O)C(OC(C)=O)C3NC(C)=O)nn2)Cc2cn(CCOCCOCCOCCOC3OC(COC(C)=O)C(OC(C)=O)C(OC(C)=O)C3NC(C)=O)nn2)c(=O)n1]

or a salt, solvate, or hydrate thereof. In some embodiments, the compound is of the Formula:

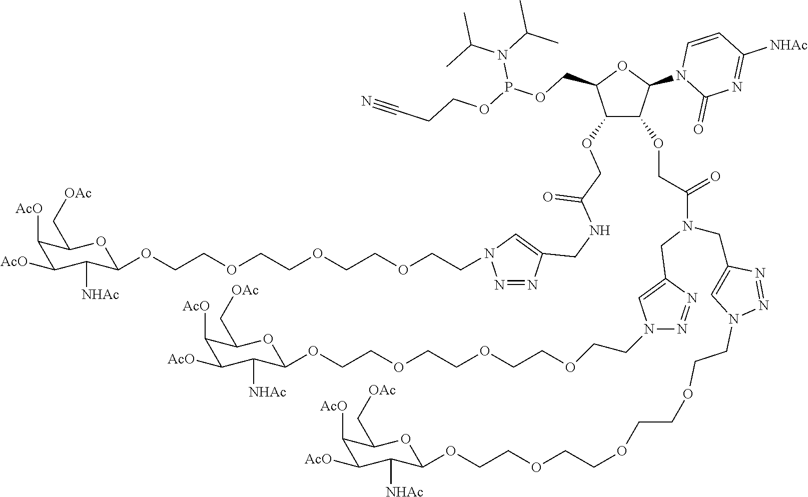 [SMILES: CC(=O)Nc1ccn([C@@H]2O[C@H](COP(OCCC#N)N(C(C)C)C(C)C)[C@@H](OCC(=O)NCc3cn(CCOCCOCCOCCOC4OC(COC(C)=O)C(OC(C)=O)C(OC(C)=O)C4NC(C)=O)nn3)[C@H]2OCC(=O)N(Cc2cn(CCOCCOCCOCCOC3OC(COC(C)=O)C(OC(C)=O)C(OC(C)=O)C3NC(C)=O)nn2)Cc2cn(CCOCCOCCOCCOC3OC(COC(C)=O)C(OC(C)=O)C(OC(C)=O)C3NC(C)=O)nn2)c(=O)n1]

or a salt, solvate, or hydrate thereof. In some embodiments, the compound is of the Formula:

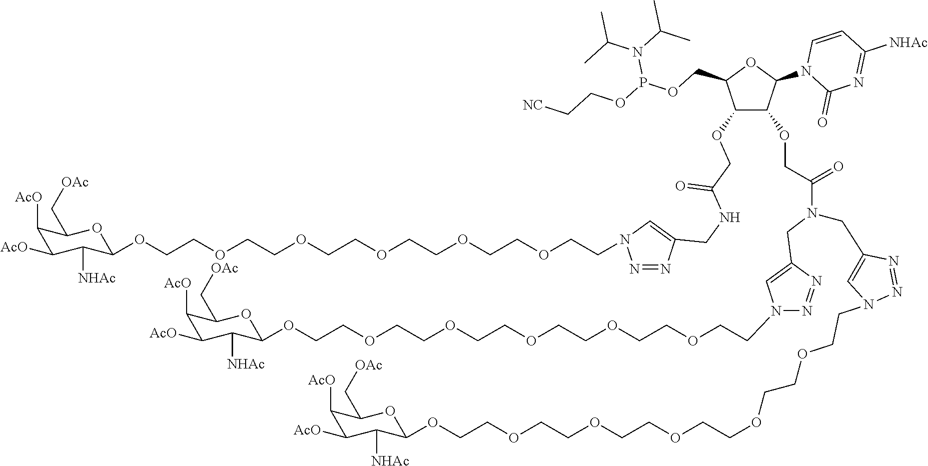 [SMILES: CC(=O)Nc1ccn([C@@H]2O[C@H](COP(OCCC#N)N(C(C)C)C(C)C)[C@@H](OCC(=O)NCc3cn(CCOCCOCCOCCOCCOCCOC4OC(COC(C)=O)C(OC(C)=O)C(OC(C)=O)C4NC(C)=O)nn3)[C@H]2OCC(=O)N(Cc2cn(CCOCCOCCOCCOCCOCCOC3OC(COC(C)=O)C(OC(C)=O)C(OC(C)=O)C3NC(C)=O)nn2)Cc2cn(CCOCCOCCOCCOCCOCCOC3OC(COC(C)=O)C(OC(C)=O)C(OC(C)=O)C3NC(C)=O)nn2)c(=O)n1]

or a salt, solvate, or hydrate thereof. In some embodiments, the compound is of the Formula:

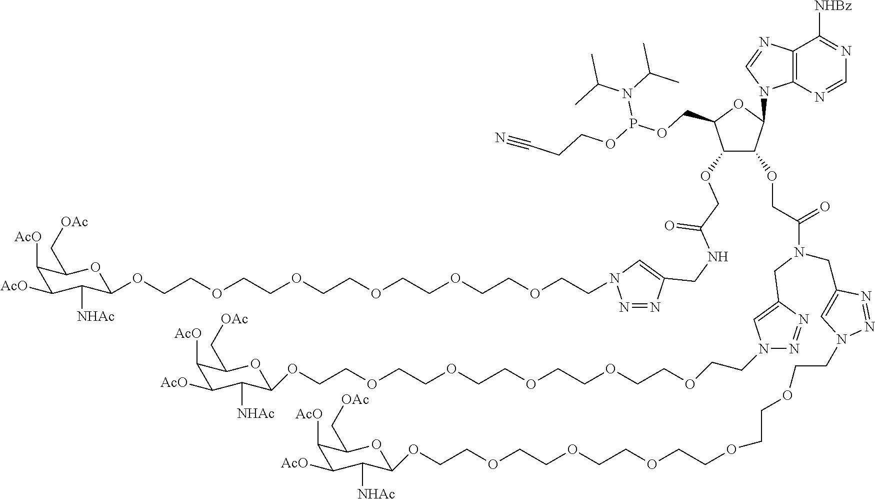

or a salt, solvate, or hydrate thereof. In some embodiments, the compound is of the Formula:

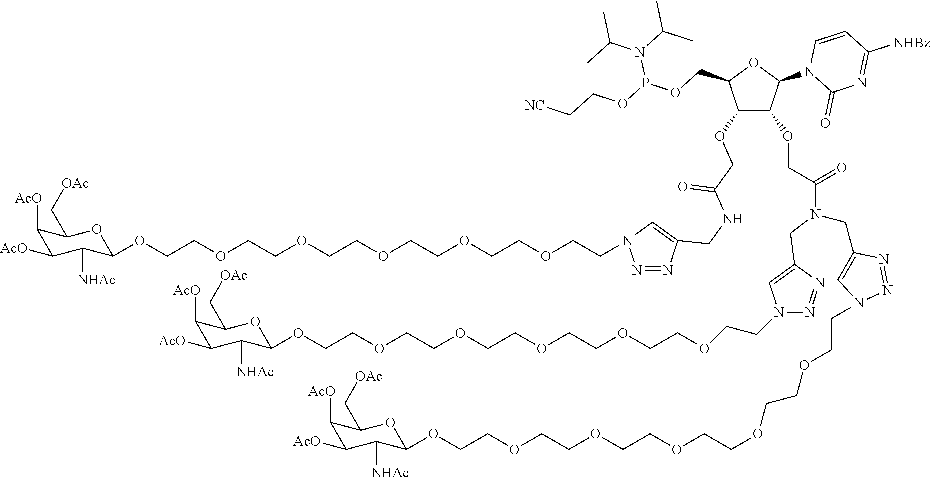

or a salt, solvate, or hydrate thereof. In some embodiments, the compound is of the Formula:

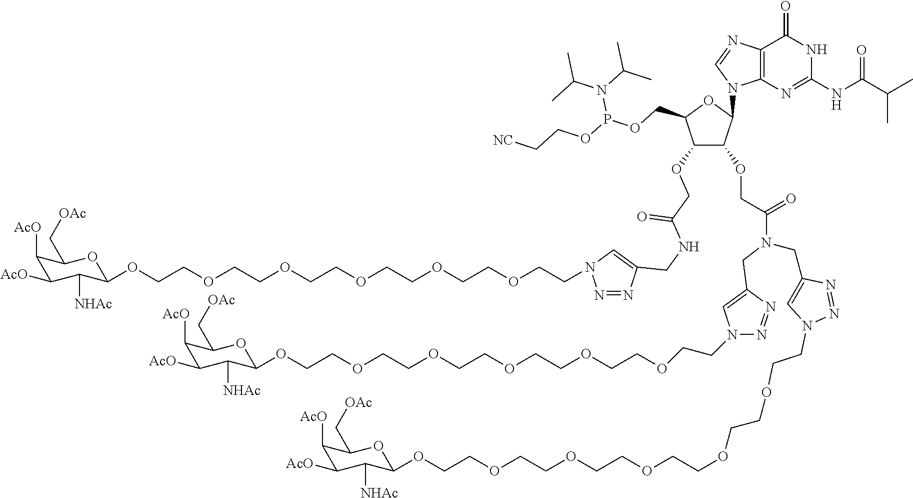 [SMILES: CC(=O)NC1C(OCCOCCOCCOCCOCCOCCn2cc(CNC(=O)CO[C@H]3[C@@H](OCC(=O)N(Cc4cn(CCOCCOCCOCCOCCOCCOC5OC(COC(C)=O)C(OC(C)=O)C(OC(C)=O)C5NC(C)=O)nn4)Cc4cn(CCOCCOCCOCCOCCOCCOC5OC(COC(C)=O)C(OC(C)=O)C(OC(C)=O)C5NC(C)=O)nn4)[C@H](n4cnc5c(=O)[nH]c(NC(=O)C(C)C)nc54)O[C@@H]3COP(OCCC#N)N(C(C)C)C(C)C)nn2)OC(COC(C)=O)C(OC(C)=O)C1OC(C)=O]

or a salt, solvate, or hydrate thereof. In some embodiments, the compound is of the Formula:

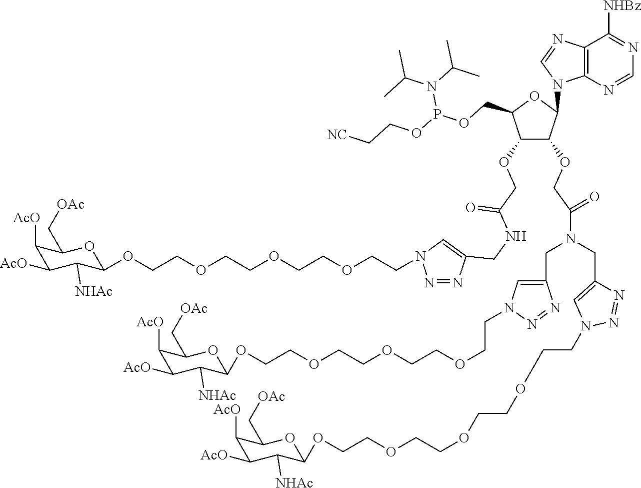

or a salt, solvate, or hydrate thereof. In some embodiments, the compound is of the Formula:

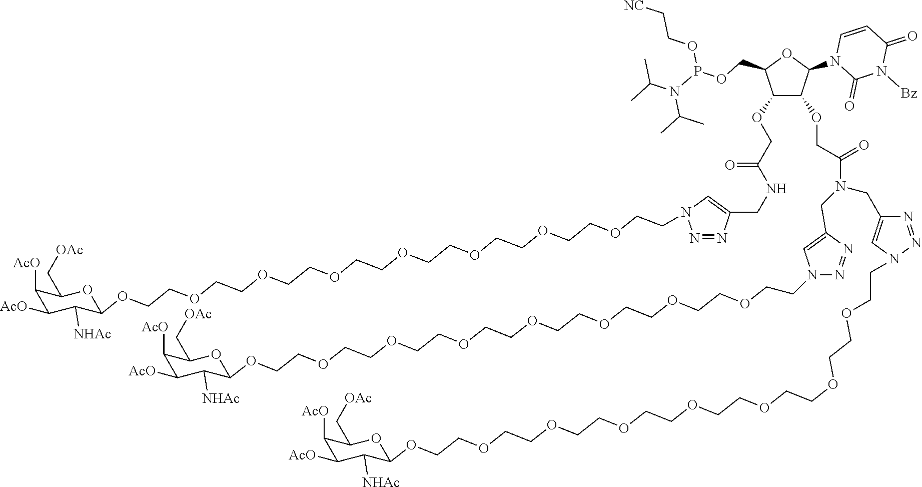

or a salt, solvate, or hydrate thereof. In some embodiments, the compound is of the Formula:

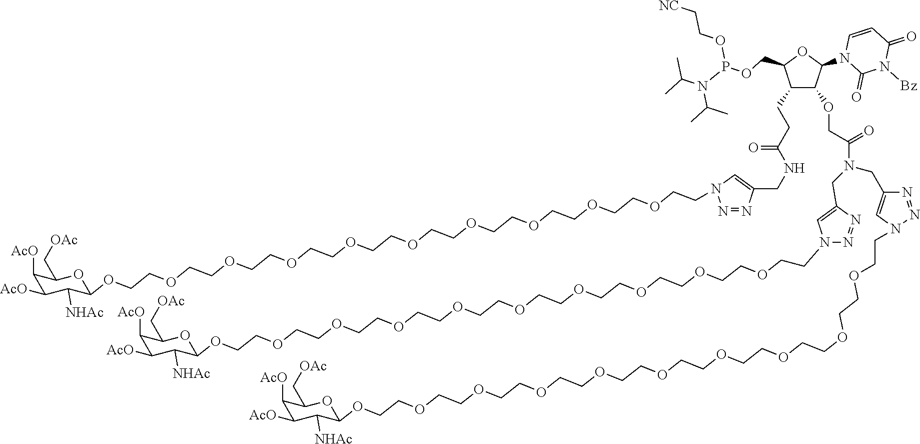

or a salt, solvate, or hydrate thereof.

In another aspect, the present disclosure provides compounds of Formula (XXIX), or salts, solvates, or hydrates thereof:

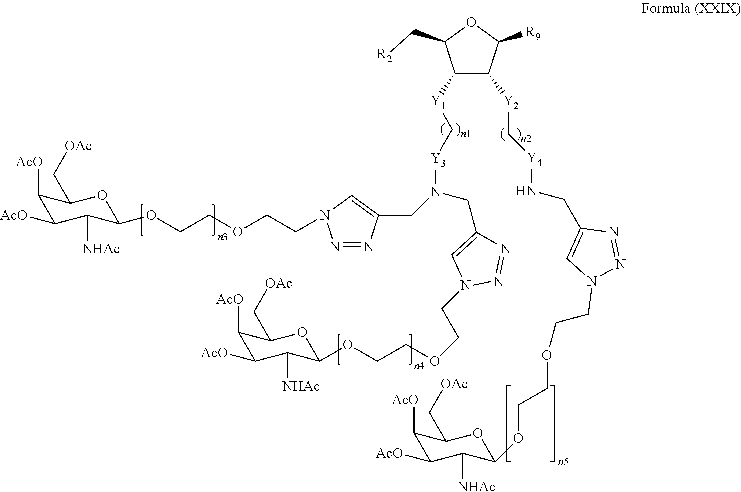

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, optionally substituted heteroaryl, optionally substituted carbocyclyl, optionally substituted heterocyclyl, or a nucleobase isostere;
R² is H, OH, O-Protecting Group (PG),

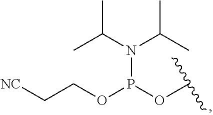 [SMILES: *OP(OCCC#N)N(C(C)C)C(C)C]

a linker, an azide, a carboxylic acid, or an amine;
Y₁ is O, optionally substituted NH, CH₂, or CH₂O;
Y₂ is O, optionally substituted NH, CH₂, or CH₂O;
Y₃ is CO, SO₂, P(O)O, CH₂—O—C(O), CH₂—NH—C(O), CH₂—NH—SO₂, or CH₂;
Y₄ is CO, SO₂, P(O)O, CH₂—O—C(O), CH₂—NH—C(O), CH₂—NH—SO₂, or CH₂;
n₂ is 0, 1, 2, 3, 4, 5, or 6;
each n₁, n₃, n₄ and n₅ is independently 0, 1, 2, 3, 4, 5, 6, 7, 8, 9, or 10.

In some embodiments, the compound is of Formula:

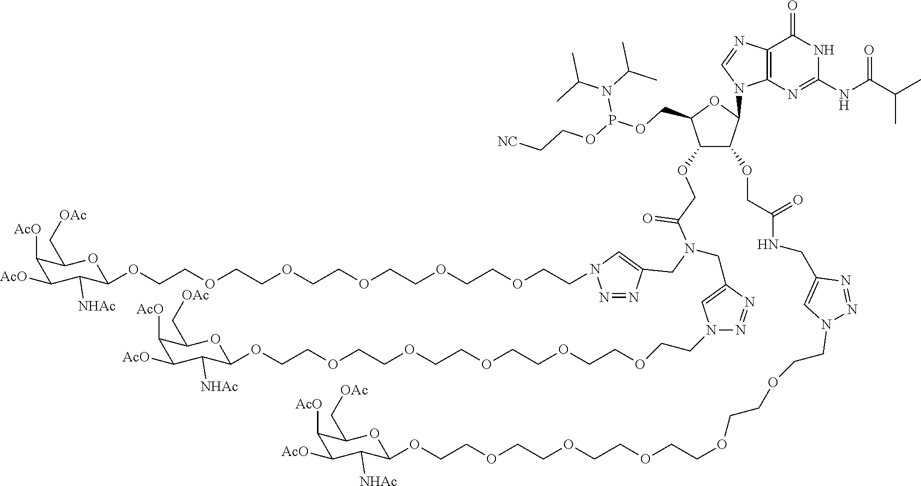 [SMILES: CC(=O)NC1C(OCCOCCOCCOCCOCCOCCn2cc(CNC(=O)CO[C@@H]3[C@H](OCC(=O)N(Cc4cn(CCOCCOCCOCCOCCOCCOC5OC(COC(C)=O)C(OC(C)=O)C(OC(C)=O)C5NC(C)=O)nn4)Cc4cn(CCOCCOCCOCCOCCOCCOC5OC(COC(C)=O)C(OC(C)=O)C(OC(C)=O)C5NC(C)=O)nn4)[C@@H](COP(OCCC#N)N(C(C)C)C(C)C)O[C@H]3n3cnc4c(=O)[nH]c(NC(=O)C(C)C)nc43)nn2)OC(COC(C)=O)C(OC(C)=O)C1OC(C)=O]

or a salt, solvate, or hydrate thereof.

In another aspect, compounds of Formula (XI), and salts, solvates, and hydrates thereof, are provided:

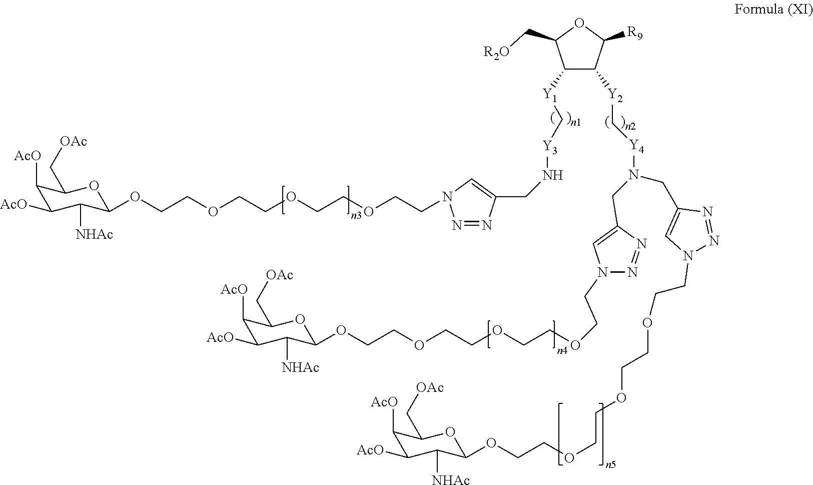

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, or optionally substituted heteroaryl;
R² is an oligonucleotide sequence;
Y₁ is O;
Y₂ is O;
Y₃ is CO, SO₂, P(O)O, CH₂—O—C(O), CH₂—NH—C(O), CH₂—NH—SO₂, or CH₂;
Y₄ is CO, SO₂, P(O)O, CH₂—O—C(O), CH₂—NH—C(O), CH₂—NH—SO₂, or CH₂;
n₂ is 1, 2, 3, 4, 5, or 6;
each n₁, n₃, n₄ and n₅ is independently 0, 1, 2, 3, 4, 5, or 6.

In another aspect, compounds of Formula (XI-a), and salts, solvates, and hydrates thereof, are provided:

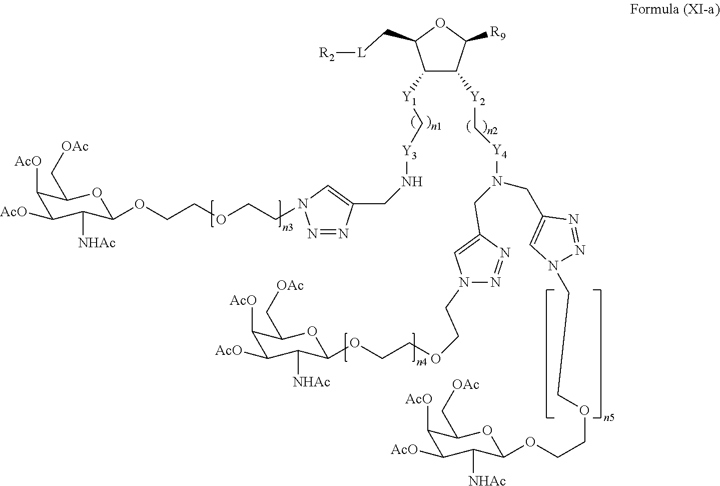

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, optionally substituted heteroaryl, optionally substituted carbocyclyl, optionally substituted heterocyclyl, or a nucleobase isostere;
L is a bond, a phosphodiester bond, a phosphorothioate bond, a triazole, a tetrazole, an amide, a reverse-amide, a carbamate, a carbonate, urea, alkyl, or heteroalkyl;
R² is an oligonucleotide sequence;
Y₁ is O, CH₂, CH₂O, or optionally substituted NH;
Y₂ is O, CH₂, CH₂O, or optionally substituted NH;
Y₃ is CO, SO₂, P(O)O, CH₂—O—C(O), CH₂—NH—C(O), CH₂—NH—SO₂, or CH₂;
Y₄ is CO, SO₂, P(O)O, CH₂—O—C(O), CH₂—NH—C(O), CH₂—NH—SO₂, or CH₂;
n₂ is 0, 1, 2, 3, 4, 5, or 6;
each n₁, n₃, n₄ and n₅ is independently 0, 1, 2, 3, 4, 5, 6, 7, 8, 9, or 10.

In another aspect, compounds of Formula (XII), and salts, solvates, and hydrates thereof, are provided:

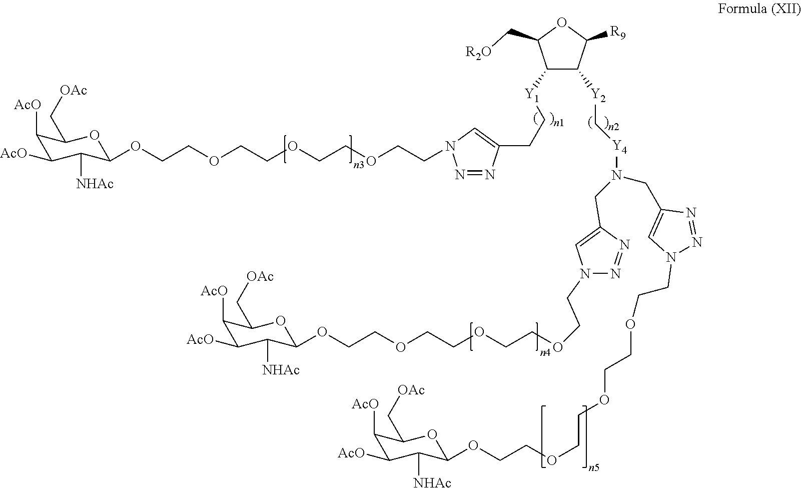

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, or optionally substituted heteroaryl;
R² is an oligonucleotide sequence;
Y₁ is O;
Y₂ is O;
Y₄ is CO, SO₂, P(O)O, CH₂—O—C(O), CH₂—NH—C(O), CH₂—NH—SO₂, or CH₂;
n₂ is 1, 2, 3, 4, 5, or 6;
each n₁, n₃, n₄ and n₅ is independently 0, 1, 2, 3, 4, 5, or 6.

In another aspect, compounds of Formula (XII-a), and salts, solvates, and hydrates thereof, are provided:

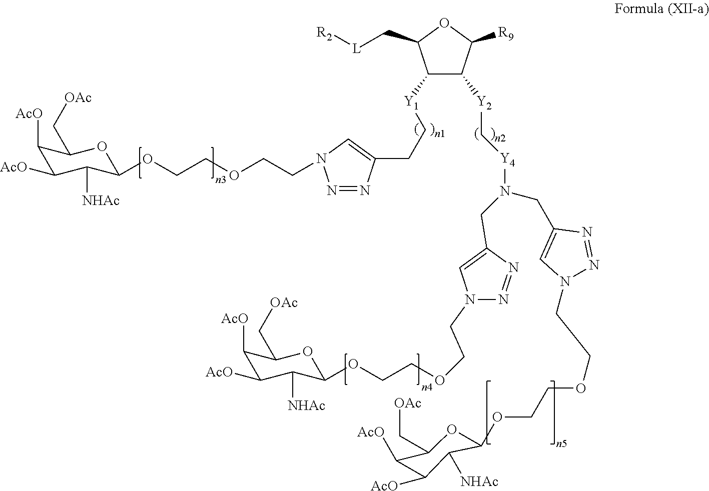

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, optionally substituted heteroaryl, optionally substituted carbocyclyl, optionally substituted heterocyclyl, or a nucleobase isostere;
L is a bond, a phosphodiester bond, a phosphorothioate bond, a triazole, a tetrazole, an amide, a reverse-amide, a carbamate, a carbonate, urea, alkyl, or heteroalkyl;
R² is an oligonucleotide sequence;
Y₁ is O, CH₂, CH₂O, or optionally substituted NH;
Y₂ is O, CH₂, CH₂O, or optionally substituted NH;
Y₄ is CO, SO₂, P(O)O, CH₂—O—C(O), CH₂—NH—C(O), CH₂—NH—SO₂, or CH₂;
n₂ is 0, 1, 2, 3, 4, 5, or 6;
each n₁, n₃, n₄ and n₅ is independently 0, 1, 2, 3, 4, 5, 6, 7, 8, 9, or 10.

In another aspect, oligonucleotides comprising a moiety of Formula (XIII), and salts, solvates, and hydrates thereof are provided:

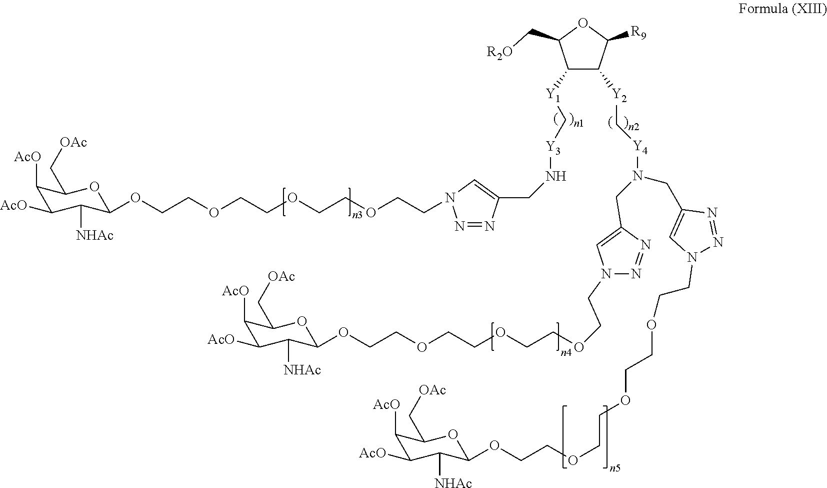

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, or optionally substituted heteroaryl;
R² is a bond;
Y₁ is O;
Y₂ is O;
Y₃ is CO, SO₂, P(O)O, CH₂—O—C(O), CH₂—NH—C(O), CH₂—NH—SO₂, or CH₂;
Y₄ is CO, SO₂, P(O)O, CH₂—O—C(O), CH₂—NH—C(O), CH₂—NH—SO₂, or CH₂;
n₂ is 1, 2, 3, 4, 5, or 6;
each n₁, n₃, n₄ and n₅ is independently 0, 1, 2, 3, 4, 5, or 6.

In another aspect, oligonucleotides comprising a moiety of Formula (XIII-a), and salts, solvates, and hydrates thereof are provided:

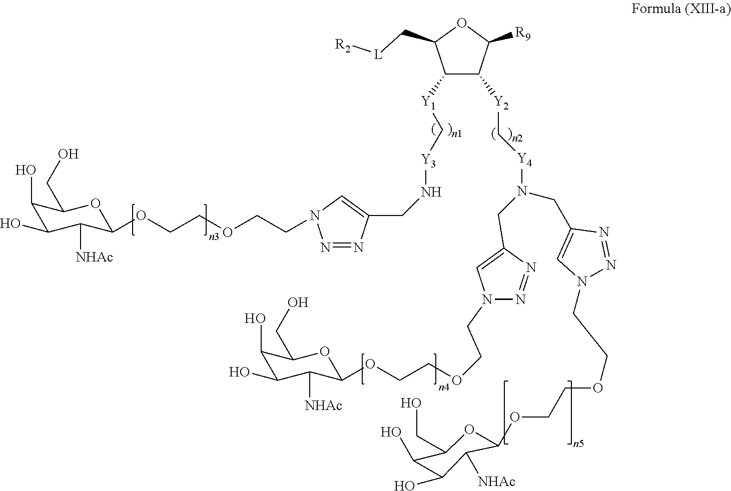

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, optionally substituted heteroaryl, optionally substituted carbocyclyl, optionally substituted heterocyclyl, or a nucleobase isostere;
L is a bond, a phosphodiester bond, a phosphorothioate bond, a triazole, a tetrazole, an amide, a reverse-amide, a carbamate, a carbonate, urea, alkyl, or heteroalkyl;
R² is a bond to an oligonucleotide sequence;
Y₁ is O, CH₂, CH₂O, or optionally substituted NH;
Y₂ is O, CH₂, CH₂O, or optionally substituted NH;
Y₃ is CO, SO₂, P(O)O, CH₂—O—C(O), CH₂—NH—C(O), CH₂—NH—SO₂, or CH₂;
Y₄ is CO, SO₂, P(O)O, CH₂—O—C(O), CH₂—NH—C(O), CH₂—NH—SO₂, or CH₂;
n₂ is 0, 1, 2, 3, 4, 5, or 6;
each n₁, n₃, n₄ and n₅ is independently 0, 1, 2, 3, 4, 5, 6, 7, 8, 9, or 10.

In another aspect, oligonucleotides comprising a moiety of Formula (XIV), and salts, solvates, and hydrates thereof, are provided:

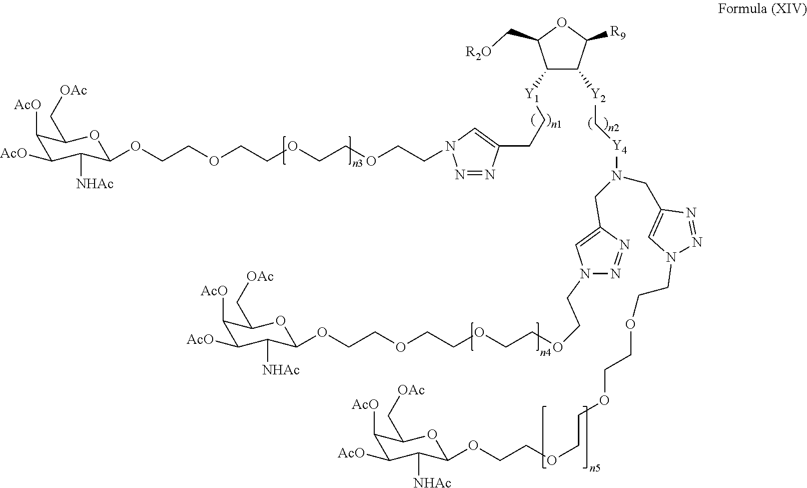

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, or optionally substituted heteroaryl;
R² is a bond;
Y₁ is O;
Y₂ is O;
Y₄ is CO, SO₂, P(O)O, CH₂—O—C(O), CH₂—NH—C(O), CH₂—NH—SO₂, or CH₂;
n₂ is 1, 2, 3, 4, 5, or 6;
each n₁, n₃, n₄ and n₅ is independently 0, 1, 2, 3, 4, 5, or 6.

In another aspect, oligonucleotides comprising a moiety of Formula (XIV-a), and salts, solvates, and hydrates thereof, are provided:

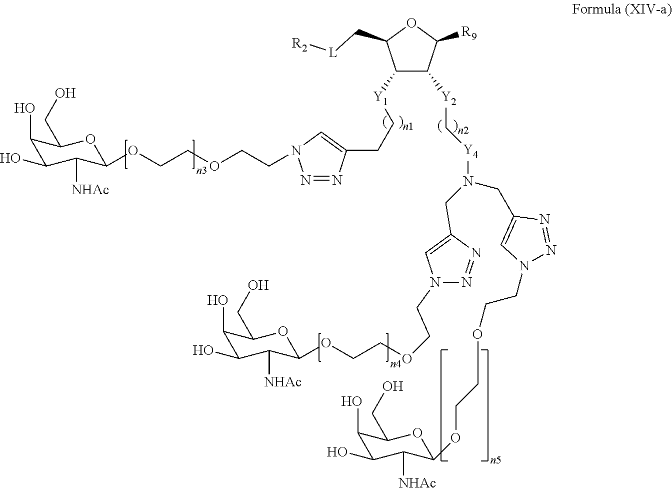

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, optionally substituted heteroaryl, optionally substituted carbocyclyl, optionally substituted heterocyclyl, or a nucleobase isostere;
L is a bond, a phosphodiester bond, a phosphorothioate bond, a triazole, a tetrazole, an amide, a reverse-amide, a carbamate, a carbonate, urea, alkyl, or heteroalkyl;
R² is a bond to an oligonucleotide sequence;
Y₁ is O, CH₂, CH₂O, or optionally substituted NH;
Y₂ is O, CH₂, CH₂O, or optionally substituted NH;
Y₄ is CO, SO₂, P(O)O, CH₂—O—C(O), CH₂—NH—C(O), CH₂—NH—SO₂, or CH₂;
n₂ is 0, 1, 2, 3, 4, 5, or 6,
each n₁, n₃, n₄ and n₅ is independently 0, 1, 2, 3, 4, 5, 6, 7, 8, 9, or 10.

In another aspect, compounds of Formula (XV), and salts, solvates, and hydrates thereof, are provided:

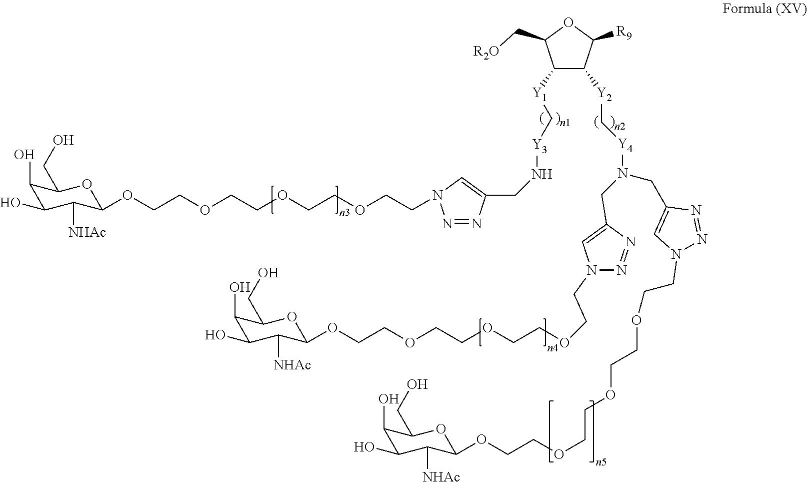

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, or optionally substituted heteroaryl;
R² is an oligonucleotide sequence;
Y₁ is O;
Y₂ is O;
Y₃ is CO, SO₂, P(O)O, CH₂—O—C(O), CH₂—NH—C(O), CH₂—NH—SO₂, or CH₂;
Y₄ is CO, SO₂, P(O)O, CH₂—O—C(O), CH₂—NH—C(O), CH₂—NH—SO₂, or CH₂;
n₂ is 1, 2, 3, 4, 5, or 6,
each n₁, n₃, n₄ and n₅ is independently 0, 1, 2, 3, 4, 5, or 6.

In another aspect, compounds of Formula (XV-a), and salts, solvates, and hydrates thereof, are provided:

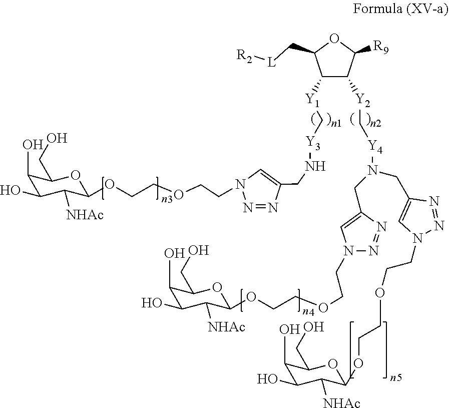

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, optionally substituted heteroaryl, optionally substituted carbocyclyl, optionally substituted heterocyclyl, or a nucleobase isostere;
L is a bond, a phosphodiester bond, a phosphorothioate bond, a triazole, a tetrazole, an amide, a reverse-amide, a carbamate, a carbonate, urea, alkyl, or heteroalkyl;
R² is an oligonucleotide sequence;
Y₁ is O, CH₂, CH₂O, or optionally substituted NH;
Y₂ is O, CH₂, CH₂O, or optionally substituted NH;
Y₃ is CO, SO₂, P(O)O, CH₂—O—C(O), CH₂—NH—C(O), CH₂—NH—SO₂, or CH₂;
Y₄ is CO, SO₂, P(O)O, CH₂—O—C(O), CH₂—NH—C(O), CH₂—NH—SO₂, or CH₂;
n₂ is 0, 1, 2, 3, 4, 5, or 6;
each n₁, n₃, n₄ and n₅ is independently 0, 1, 2, 3, 4, 5, 6, 7, 8, 9, or 10.

In another aspect, compounds of Formula (XVI), and salts, solvates, and hydrates thereof, are provided:

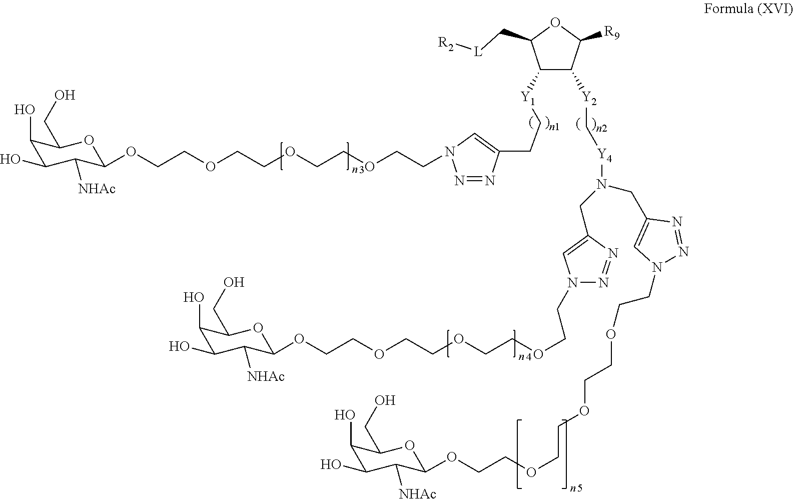

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, optionally substituted heteroaryl, optionally substituted carbocyclyl, optionally substituted heterocyclyl, or a nucleobase isostere;
L is a bond, a phosphodiester bond, a phosphorothioate bond, a triazole, a tetrazole, an amide, a reverse-amide, a carbamate, a carbonate, urea, alkyl, or heteroalkyl;
R² is an oligonucleotide sequence;
Y₁ is O, CH₂, CH₂O, or optionally substituted NH;
Y₂ is O, CH₂, CH₂O, or optionally substituted NH;
Y₄ is CO, SO₂, P(O)O, CH₂—O—C(O), CH₂—NH—C(O), CH₂—NH—SO₂, or CH₂;
n₂ is 0, 1, 2, 3, 4, 5, or 6;
each n₁, n₃, n₄ and n₅ is independently 0, 1, 2, 3, 4, 5, or 6.

In another aspect, compounds of Formula (XVI-a), and salts, solvates, and hydrates thereof, are provided:

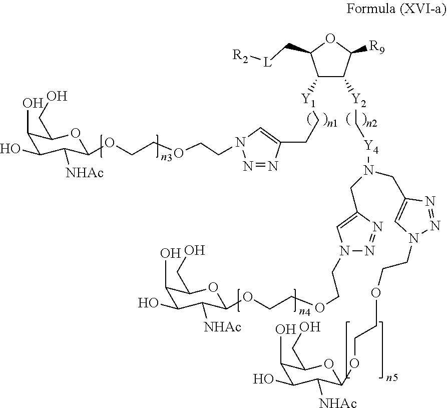

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, optionally substituted heteroaryl, optionally substituted carbocyclyl, optionally substituted heterocyclyl, or a nucleobase isostere;
L is a bond, a phosphodiester bond, a phosphorothioate bond, a triazole, a tetrazole, an amide, a reverse-amide, a carbamate, a carbonate, urea, alkyl, or heteroalkyl;
R² is an oligonucleotide sequence;
Y₁ is O, CH₂, CH₂O, or optionally substituted NH;
Y₂ is O, CH₂, CH₂O, or optionally substituted NH;
Y₄ is CO, SO₂, P(O)O, CH₂—O—C(O), CH₂—NH—C(O), CH₂—NH—SO₂, or CH₂;
n₂ is 0, 1, 2, 3, 4, 5, or 6;
each n₁, n₃, n₄ and n₅ is independently 0, 1, 2, 3, 4, 5, 6, 7, 8, 9, or 10.

In some embodiments, R⁹ is uracil. In some embodiments, R⁹ is cytosine. In some embodiments, R⁹ is adenine. In some embodiments, R⁹ is guanine.

In some embodiments, Y₁ is O. In some embodiments, Y₂ is O. In certain embodiments, both Y₁ and Y₂ are O. In some embodiments, Y₃ is CO. In some embodiments, Y₄ is CO. In certain embodiments, both Y₃ and Y₄ are CO. In certain embodiments, Y₁ and Y₂ are O, and Y₃ and Y₄ are CO.

In some embodiments, n₃ is 1. In some embodiments, n₃ is 3. In some embodiments, n₃ is 5. In some embodiments, n₃ is 7. In some embodiments, n₄ is 1. In some embodiments, n₄ is 3. In some embodiments, n₄ is 5. In some embodiments, n₄ is 7. In some embodiments, n₅ is 1. In some embodiments, n₅ is 3. In some embodiments, n₅ is 5. In some embodiments, n₅ is 7. In certain embodiments, two of n₃, n₄, and n₅ are 1. In certain embodiments, all three of n₃, n₄, and n₅ are 1.

In some embodiments, the compound is of the formula:

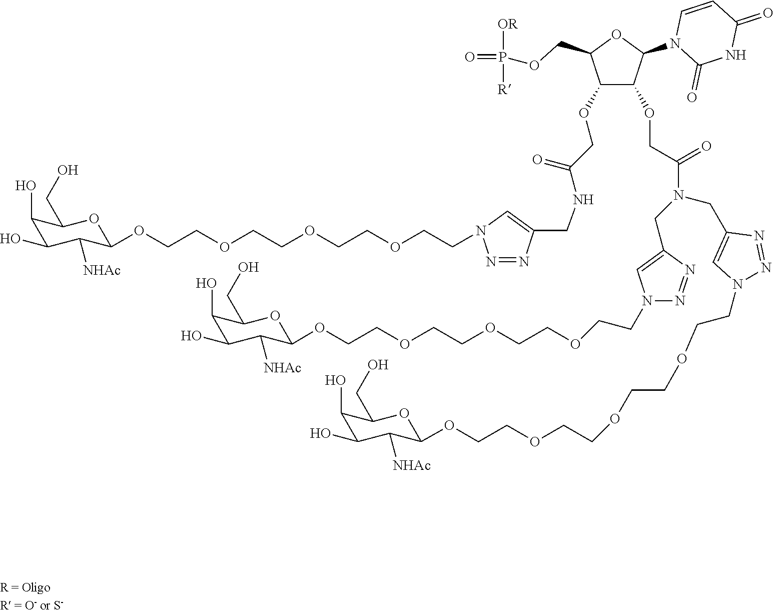

or a salt, solvate, or hydrate thereof. In some embodiments, the compound is of the formula:

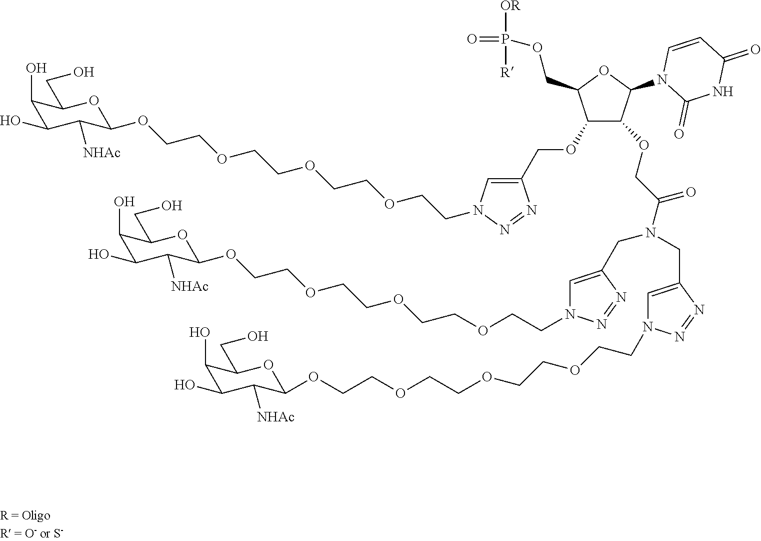

or a salt, solvate, or hydrate thereof. In some embodiments, the compound is of the formula:

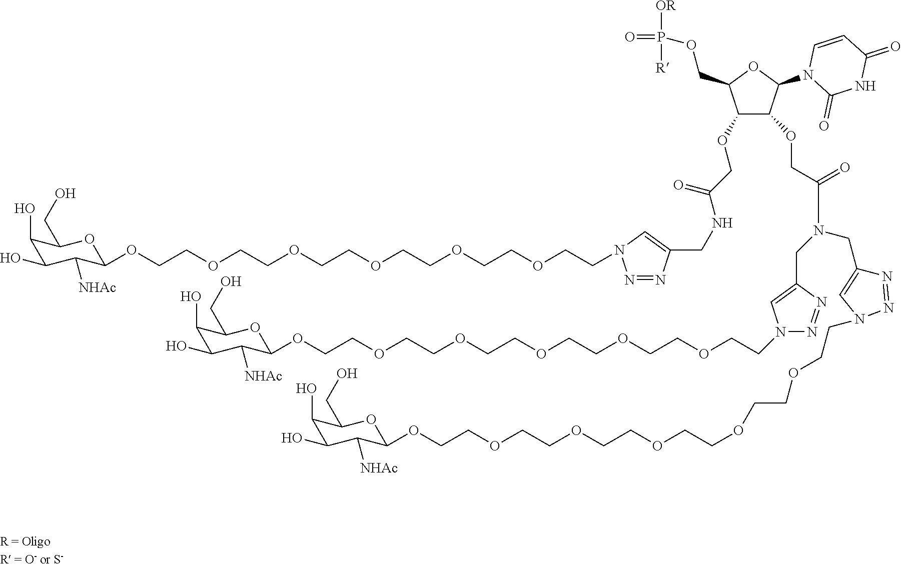

or a salt, solvate, or hydrate thereof. In some embodiments, the compound is of the formula:

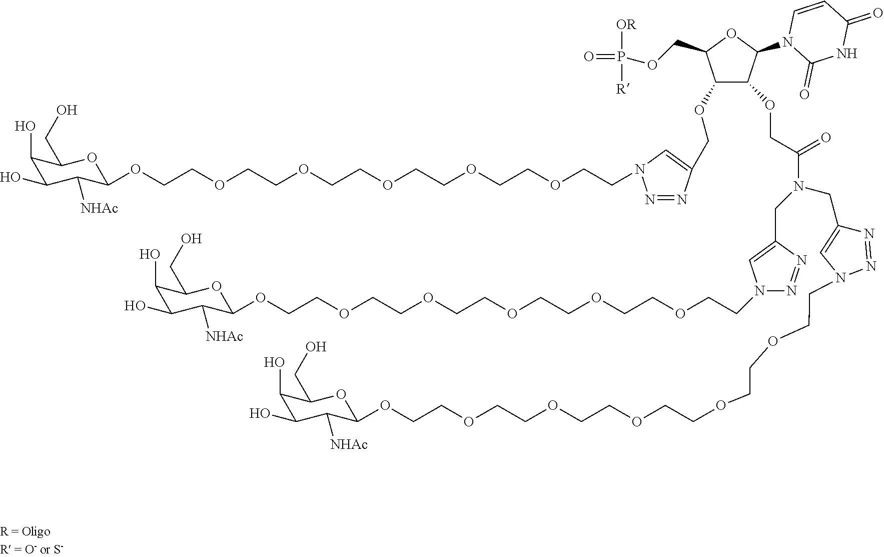

or a salt, solvate, or hydrate thereof. In some embodiments, the compound is of the formula:

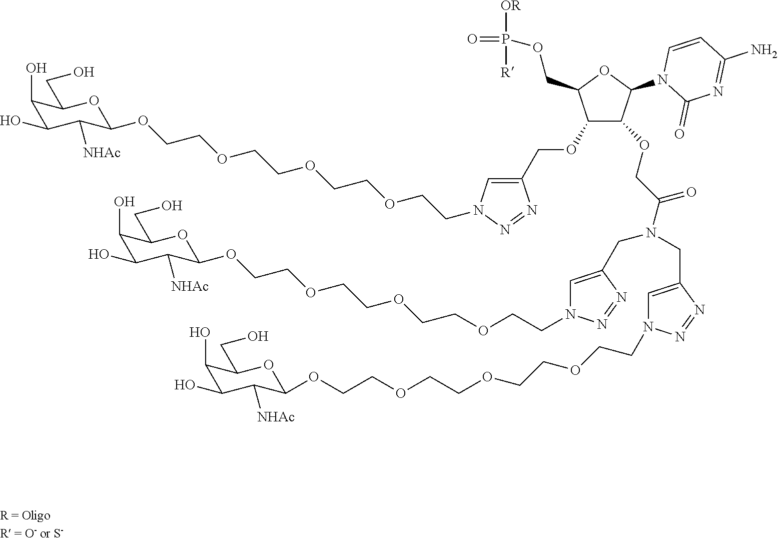

or a salt, solvate, or hydrate thereof. In some embodiments, the compound is of the formula:

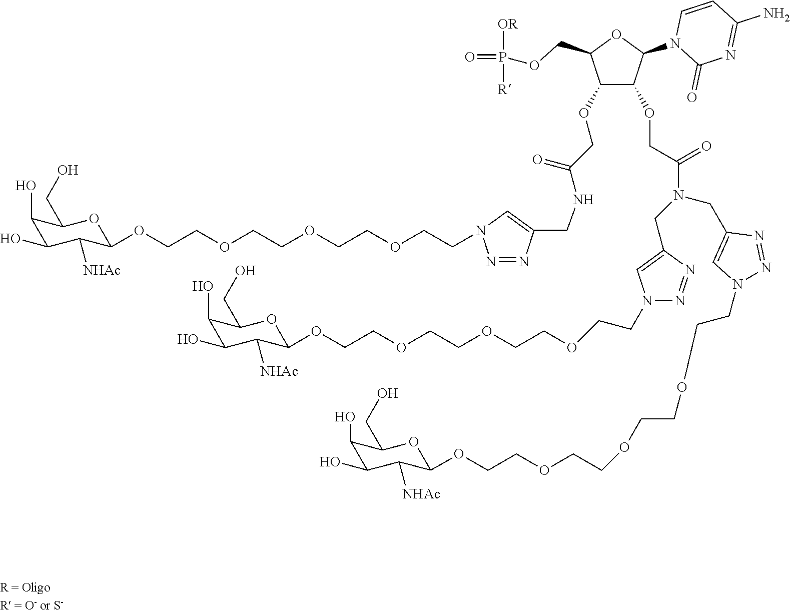

or a salt, solvate, or hydrate thereof. In some embodiments, the compound is of the formula:

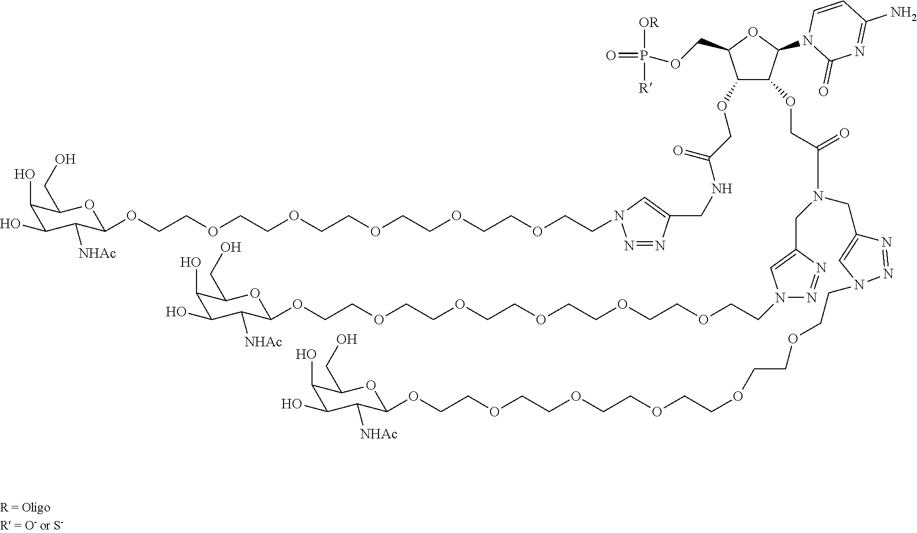

or a salt, solvate, or hydrate thereof. In some embodiments, the compound is of the formula:

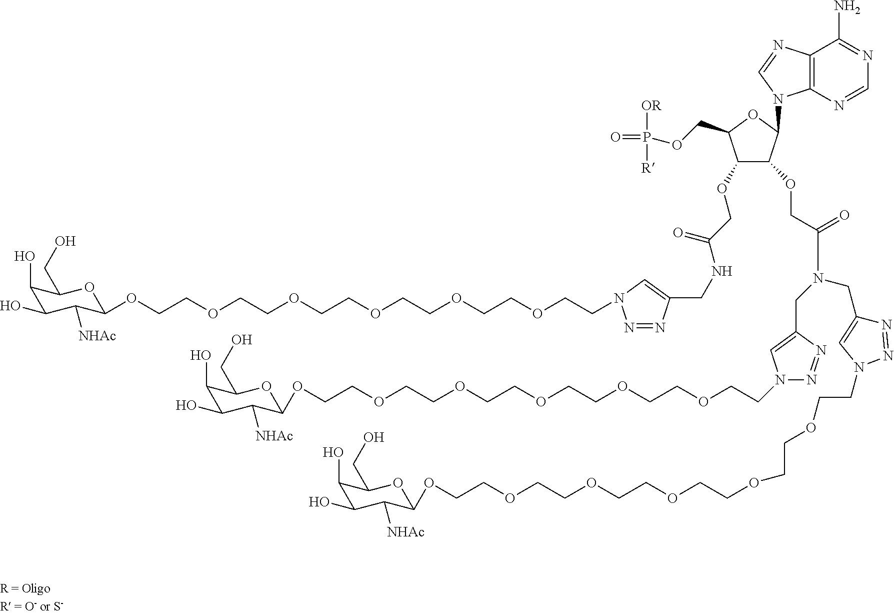

or a salt, solvate, or hydrate thereof. In some embodiments, the compound is of the formula:

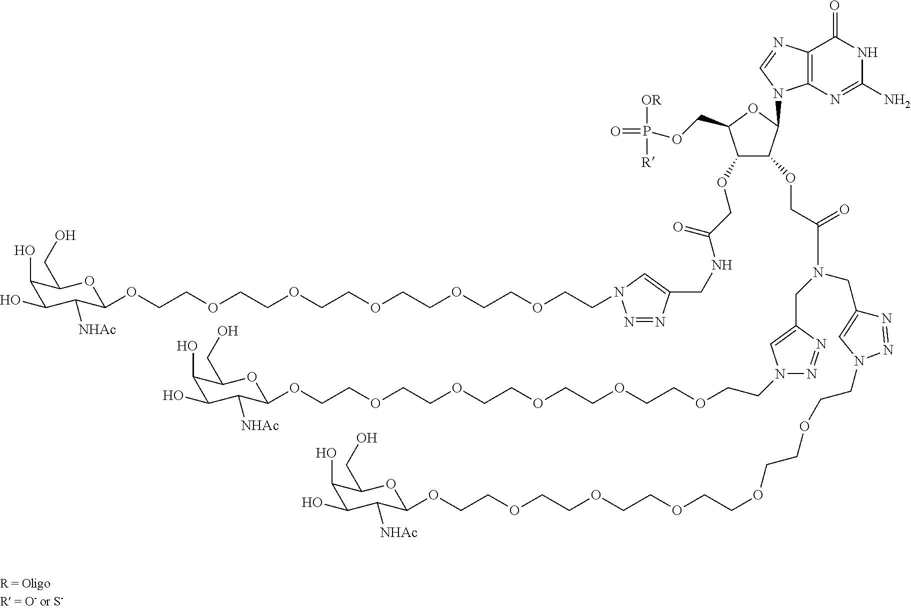

or a salt, solvate, or hydrate thereof. In some embodiments, the compound is of the formula:

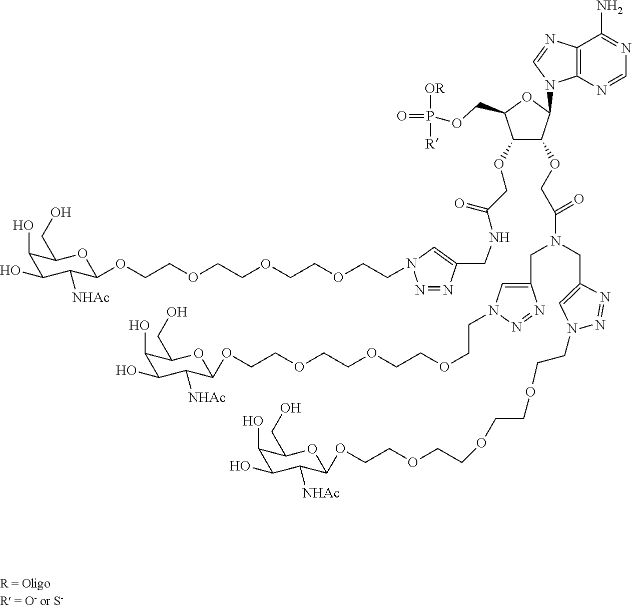

or a salt, solvate, or hydrate thereof. In some embodiments, the compound is of the formula:

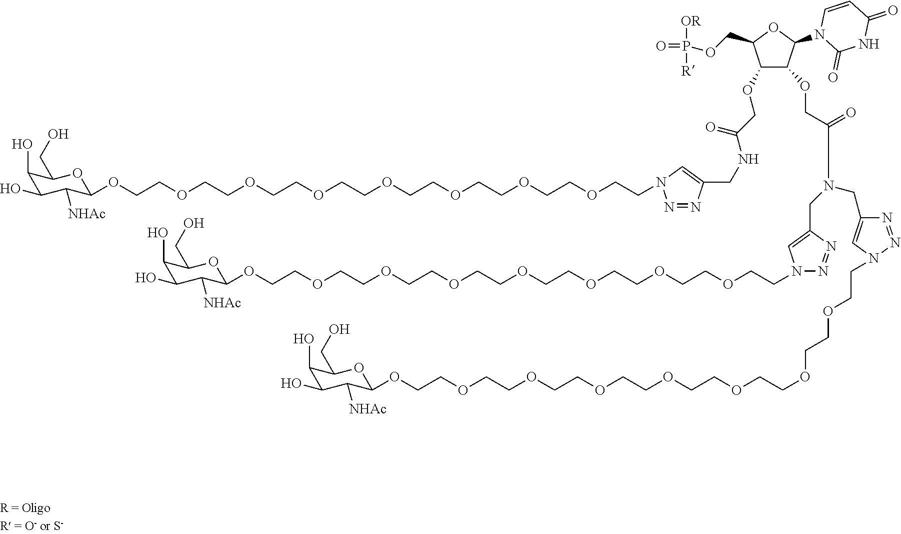

or a salt, solvate, or hydrate thereof. In some embodiments, the compound is of the formula:

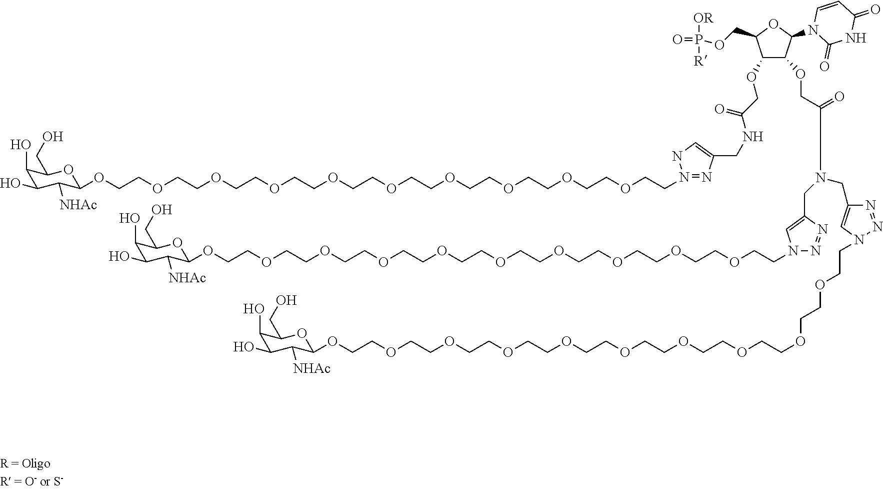

or a salt, solvate, or hydrate thereof.

In another aspect, compounds of Formula (XXX), and salts, solvates, and hydrates thereof, are provided:

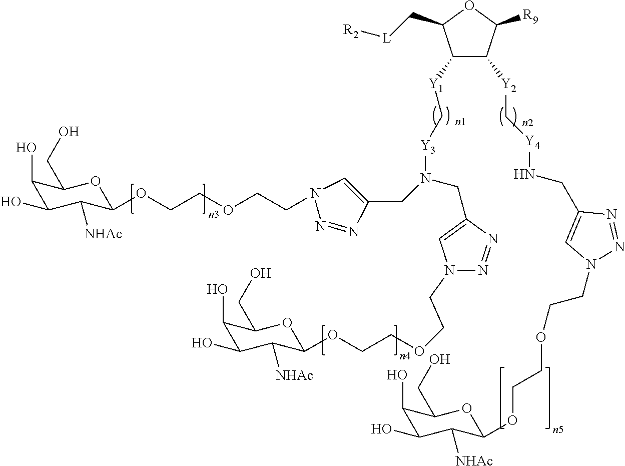

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, optionally substituted heteroaryl, optionally substituted carbocyclyl, optionally substituted heterocyclyl, or a nucleobase isostere;
L is a bond, a phosphodiester bond, a phosphorothioate bond, a triazole, a tetrazole, an amide, a reverse-amide, a carbamate, a carbonate, urea, alkyl, or heteroalkyl;
R² is an oligonucleotide sequence;
Y₁ is O, CH₂, CH₂O, or optionally substituted NH;
Y₂ is O, CH₂, CH₂O, or optionally substituted NH;
Y₃ is CO, SO₂, P(O)O, CH₂—O—C(O), CH₂—NH—C(O), CH₂—NH—SO₂, or CH₂;
Y₄ is CO, SO₂, P(O)O, CH₂—O—C(O), CH₂—NH—C(O), CH₂—NH—SO₂, or CH₂;
n₂ is 0, 1, 2, 3, 4, 5, or 6;
each n₁, n₃, n₄ and n₅ is independently 0, 1, 2, 3, 4, 5, 6, 7, 8, 9, or 10.

In some embodiments, the compound is of the formula:

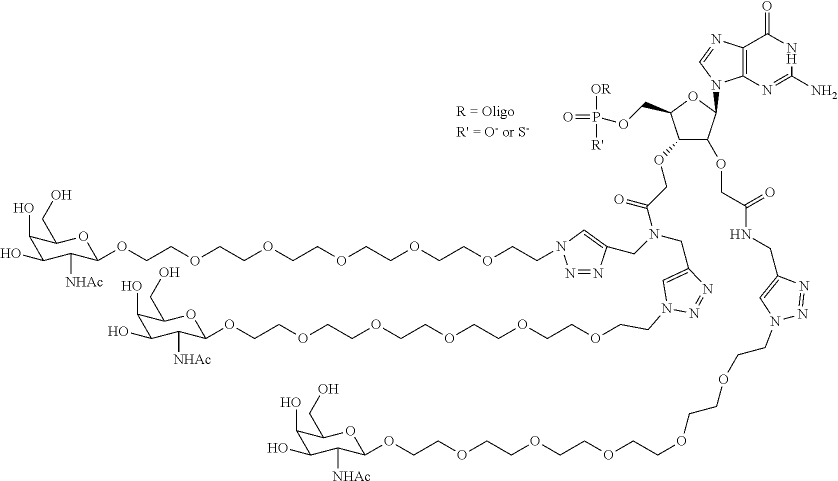

or a salt, solvate, or hydrate thereof.

In another aspect, oligonucleotides comprising a moiety of Formula (XVII), and salts, solvates, and hydrates thereof, are provided:

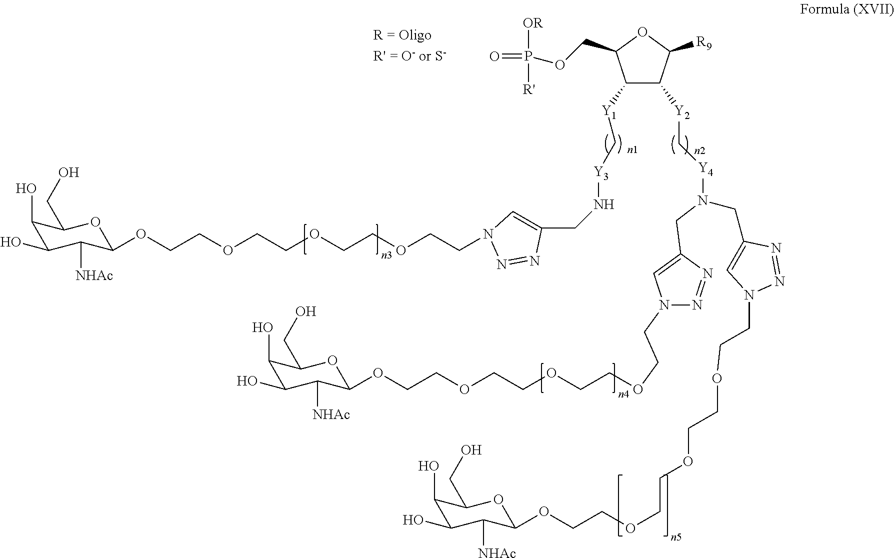

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, optionally substituted heteroaryl, optionally substituted carbocyclyl, optionally substituted heterocyclyl, or a nucleobase isostere;
R is an oligonucleotide;
R′ is O or S;
Y₁ is O, CH₂, CH₂O, or optionally substituted NH;
Y₂ is O, CH₂, CH₂O, or optionally substituted NH;
Y₃ is CO, SO₂, P(O)O, CH₂—O—C(O), CH₂—NH—C(O), CH₂—NH—SO₂, or CH₂;
Y₄ is CO, SO₂, P(O)O, CH₂—O—C(O), CH₂—NH—C(O), CH₂—NH—SO₂, or CH₂;
n₂ is 0, 1, 2, 3, 4, 5, or 6;
each n₁, n₃, n₄ and n₅ is independently 0, 1, 2, 3, 4, 5, or 6.

In another aspect, oligonucleotides comprising a moiety of Formula (XVII-a), and salts, solvates, and hydrates thereof, are provided:

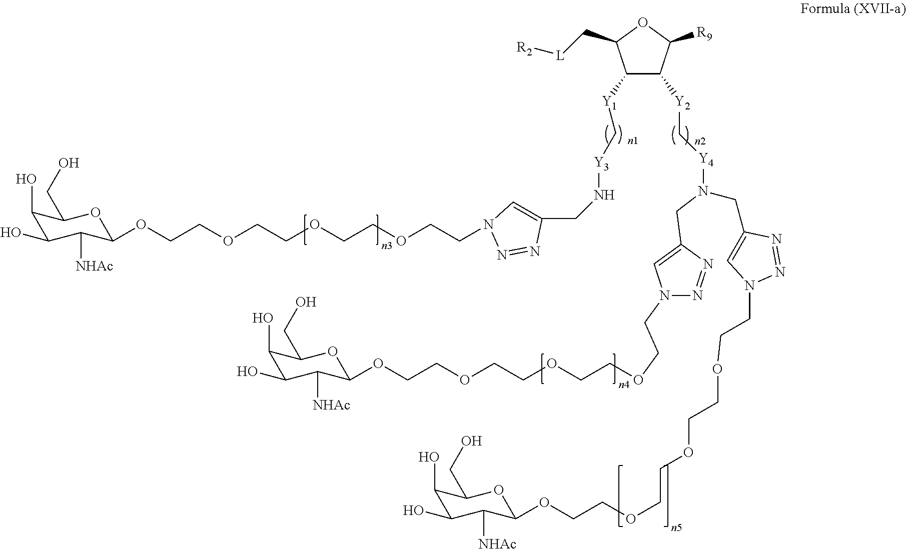

wherein.
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, optionally substituted heteroaryl, optionally substituted carbocyclyl, optionally substituted heterocyclyl, or a nucleobase isostere;
L is a bond, a phosphodiester bond, a phosphorothioate bond, a triazole, a tetrazole, an amide, a reverse-amide, a carbamate, a carbonate, urea, alkyl, or heteroalkyl;
R² is a bond to an oligonucleotide;
Y₁ is O, CH₂, CH₂O, or optionally substituted NH;
Y₂ is O, CH₂, CH₂O, or optionally substituted NH;
Y₃ is CO, SO₂, P(O)O, CH₂—O—C(O), CH₂—NH—C(O), CH₂—NH—SO₂, or CH₂;
Y₄ is CO, SO₂, P(O)O, CH₂—O—C(O), CH₂—NH—C(O), CH₂—NH—SO₂, or CH₂;
n₂ is 0, 1, 2, 3, 4, 5, or 6;
each n₁, n₃, n₄ and n₅ is independently 0, 1, 2, 3, 4, 5, 6, 7, 8, 9, or 10.

In another aspect, oligonucleotides comprising a moiety of Formula (XVIII), and salts, solvates, and hydrates thereof, are provided:

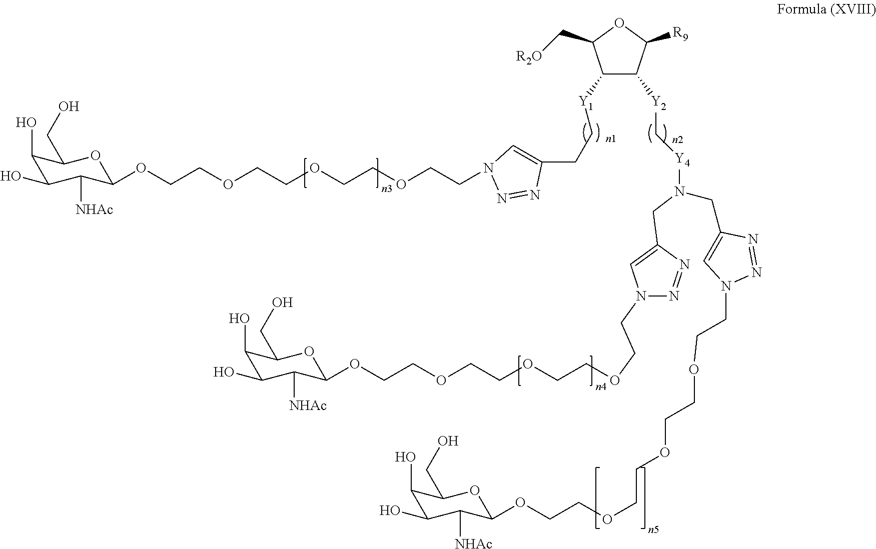

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, or optionally substituted heteroaryl;
R² is a bond;
Y₁ is O;
Y₂ is O;
Y₄ is CO, SO₂, P(O)O, CH₂—O—C(O), CH₂—NH—C(O), CH₂—NH—SO₂, or CH₂;
n₂ is 1, 2, 3, 4, 5, or 6,
each n₁, n₃, n₄ and n₅ is independently 0, 1, 2, 3, 4, 5, or 6.

In another aspect, oligonucleotides comprising a moiety of Formula (XVIII-a), and salts, solvates, and hydrates thereof, are provided:

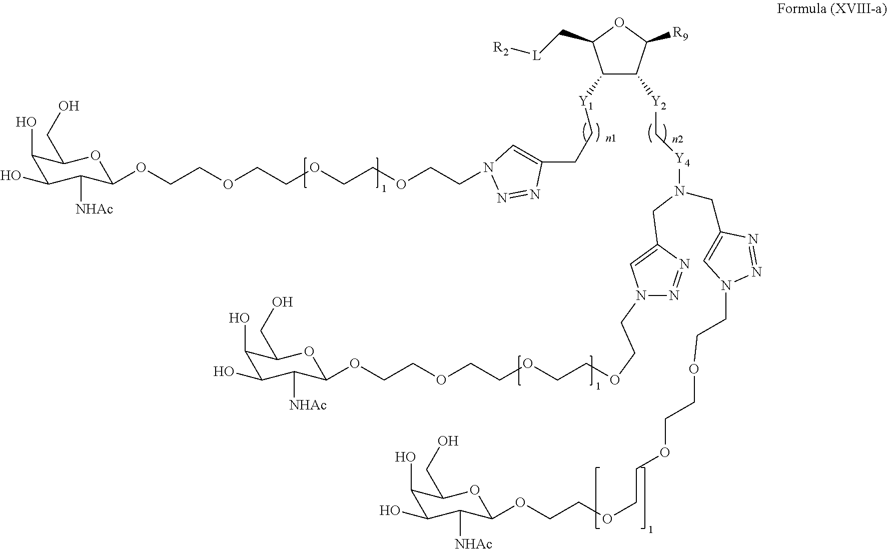

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, optionally substituted heteroalkyl, optionally substituted carbocyclyl, optionally substituted heterocyclyl, or a nucleobase isostere;
L is a bond, a phosphodiester bond, a phosphorothioate bond, a triazole, a tetrazole, an amide, a reverse-amide, a carbamate, a carbonate, urea, alkyl, or heteroalkyl;
R² is a bond to an oligonucleotide;
Y₁ is O, CH₂, CH₂O, or optionally substituted NH;
Y₂ is O, CH₂, CH₂O, or optionally substituted NH;
Y₄ is CO, SO₂, P(O)O, CH₂—O—C(O), CH₂—NH—C(O), CH₂—NH—SO₂, or CH₂.

In some embodiments, R⁹ is H, adenine, guanine, thymine, cytosine, or uracil, or adenine, guanine, thymine, cytosine, or uracil, each comprising a Protecting Group (PG). In some embodiments, R⁹ is a modified nucleobase. In some embodiments, R⁹ is optionally substituted alkyl, optionally substituted aryl, or optionally substituted heteroaryl.

In another aspect, compounds of Formula (XIX), or a salt, solvent, or hydrate thereof, are provided:

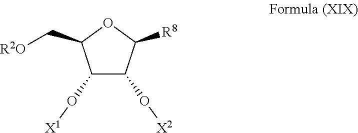

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, or optionally substituted heteroaryl;
R² is H, Protecting Group (PG), or

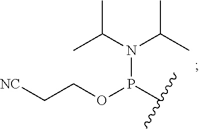 [SMILES: *P(OCCC#N)N(C(C)C)C(C)C]

X¹ is

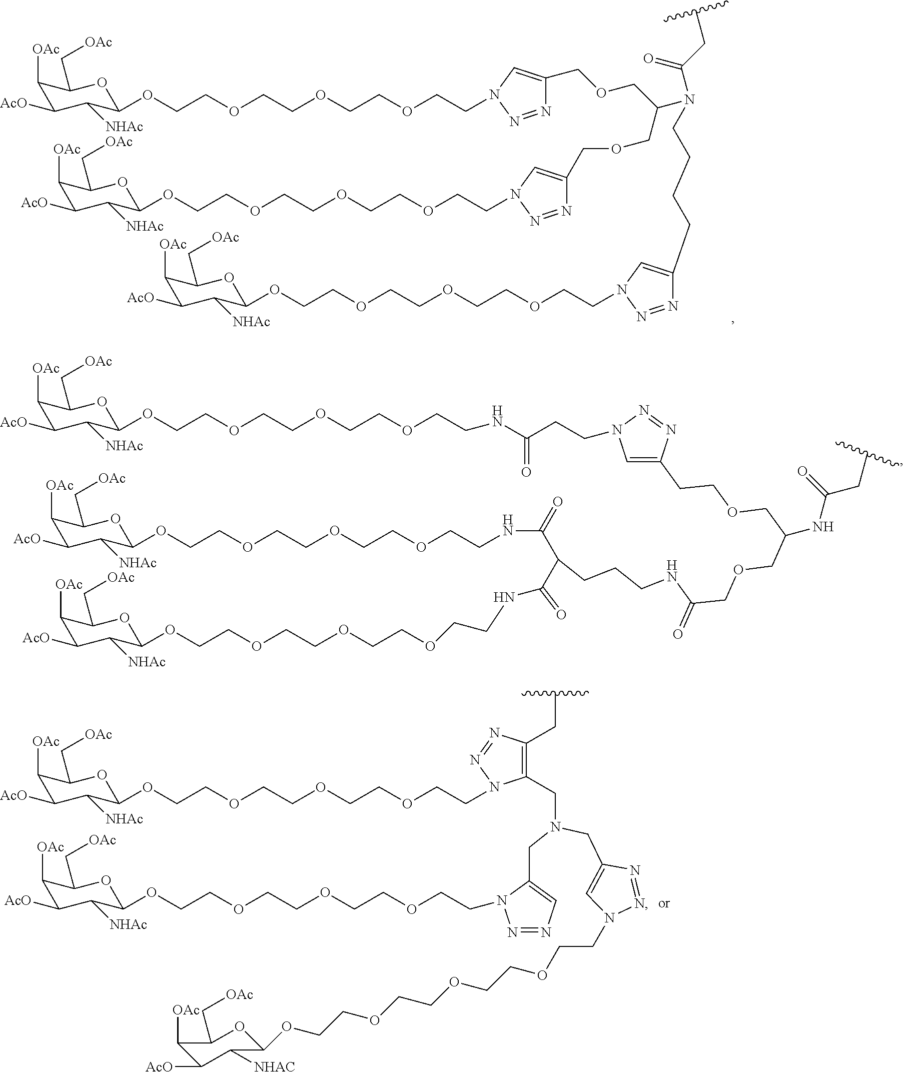

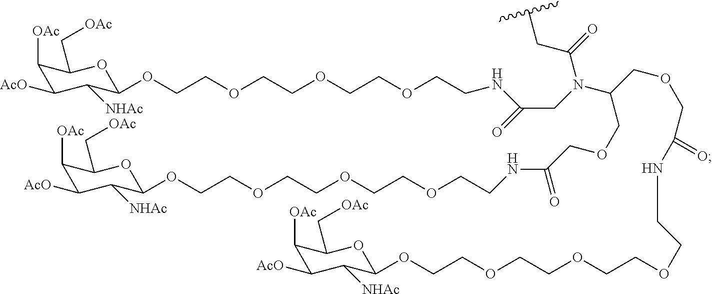 [SMILES: *CC(=O)N(CC(=O)NCCOCCOCCOCCOC1OC(COC(C)=O)C(OC(C)=O)C(OC(C)=O)C1NC(C)=O)C(COCC(=O)NCCOCCOCCOCCOC1OC(COC(C)=O)C(OC(C)=O)C(OC(C)=O)C1NC(C)=O)COCC(=O)NCCOCCOCCOCCOC1OC(COC(C)=O)C(OC(C)=O)C(OC(C)=O)C1NC(C)=O]

and
X² is H, alkyl, or Protecting Group (PG).

In another aspect, compounds of Formula (XX), or a salt, solvent, or hydrate thereof, are provided:

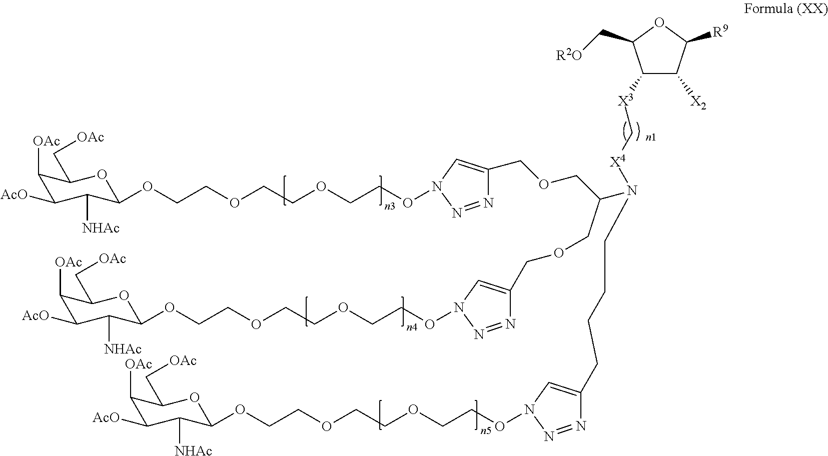

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, or optionally substituted heteroaryl;
R² is H, Protecting Group (PG), or

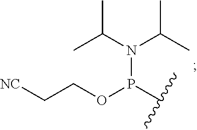 [SMILES: *P(OCCC#N)N(C(C)C)C(C)C]

X² is H, halogen, OH, O-alkyl, O-heteroalkyl, or O-Protecting Group (PG);
X³ is O, CH₂, CH₂O, or optionally substituted NH;
X⁴ is CO, SO₂, P(O)O, CH₂—O—C(O), CH₂—NH—C(O), CH₂—NH—SO₂, or CH₂; and
each n₁, n₃, n₄ and n₅ is independently 0, 1, 2, 3, 4, 5, or 6.

In another aspect, compounds of Formula (XXI), or a salt, solvate, or hydrate thereof, are provided:

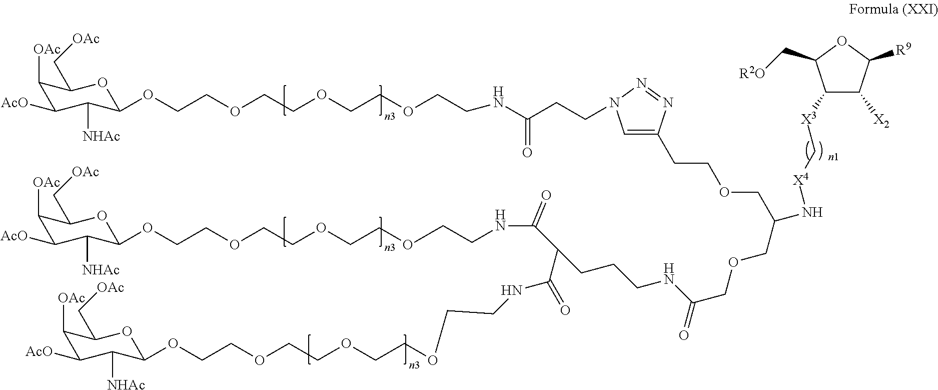

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, or optionally substituted heteroaryl;
R² is H, Protecting Group (PG), or

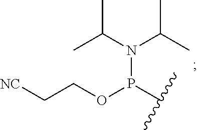 [SMILES: *P(OCCC#N)N(C(C)C)C(C)C]

X² is H, halogen, OH, O-alkyl, O-heteroalkyl, or Protecting Group (PG);
X³ is O, CH₂, CH₂O, or optionally substituted NH;
X⁴ is CO, SO₂, P(O)O, CH₂—O—C(O), CH₂—NH—C(O), CH₂—NH—SO₂, or CH₂; and
each n₁, n₃, n₄ and n₅ is independently 0, 1, 2, 3, 4, 5, or 6.

In another aspect, compounds of Formula (XXII), or a salt, solvate, or hydrate thereof, are provided:

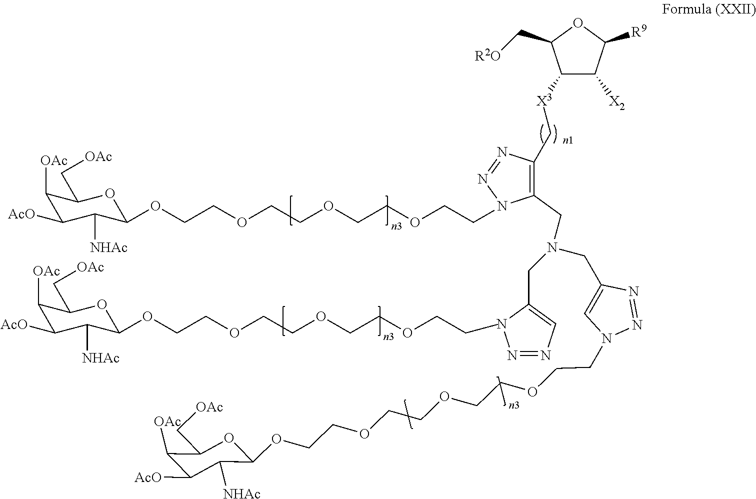

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, or optionally substituted heteroaryl;
R² is H, Protecting Group (PG), or

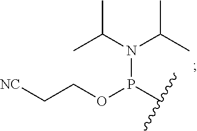 [SMILES: *P(OCCC#N)N(C(C)C)C(C)C]

X² is H, halogen, OH, O-alkyl, O-heteroalkyl, or O-Protecting Group (PG);
X³ is O, CH₂, CH₂O, or optionally substituted NH;
each n₁, n₃, n₄ and n₅ is independently 0, 1, 2, 3, 4, 5, or 6.

In another aspect, compounds of Formula (XXIII), or a salt, solvate, or hydrate thereof, are provided:

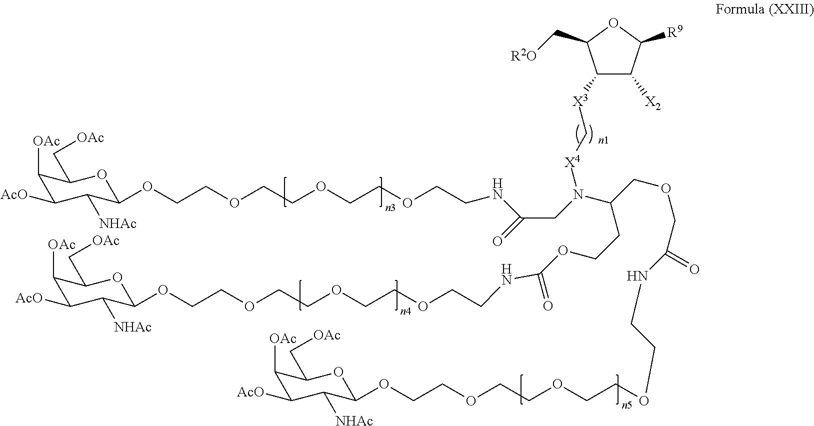

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, or optionally substituted heteroaryl;
R² is H, Protecting Group (PG), or

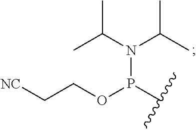 [SMILES: *P(OCCC#N)N(C(C)C)C(C)C]

X² is H, halogen, OH, O-alkyl, O-heteroalkyl, or O-Protecting Group (PG);
X³ is O, CH₂, CH₂O, or optionally substituted NH;
X⁴ is CO, SO₂, P(O)O, CH₂—O—C(O), CH₂—NH—C(O), CH₂—NH—SO₂, or CH₂; and
each n₁, n₃, n₄ and n₅ is independently 0, 1, 2, 3, 4, 5, or 6.

In some embodiments, the compound is:

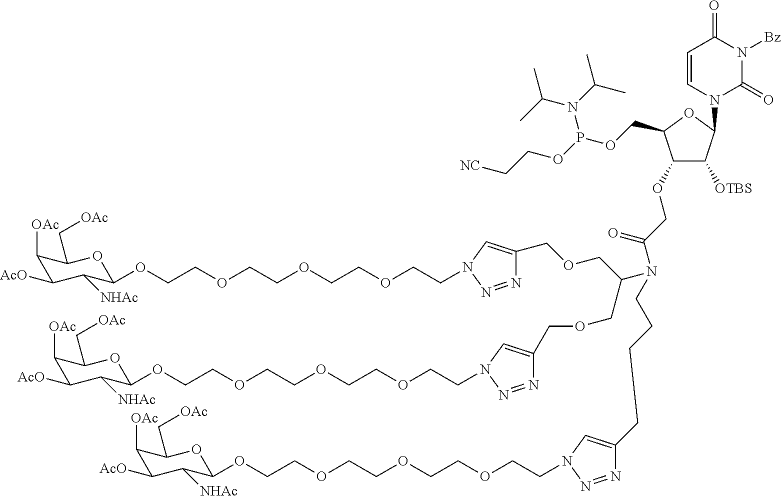

or a salt, solvate, or hydrate thereof. In some embodiments, the compound is:

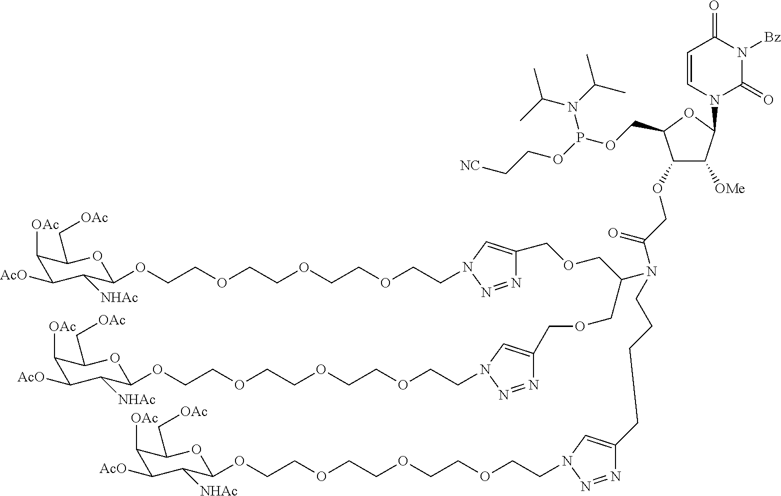

or a salt, solvate, or hydrate thereof. In some embodiments, the compound is:

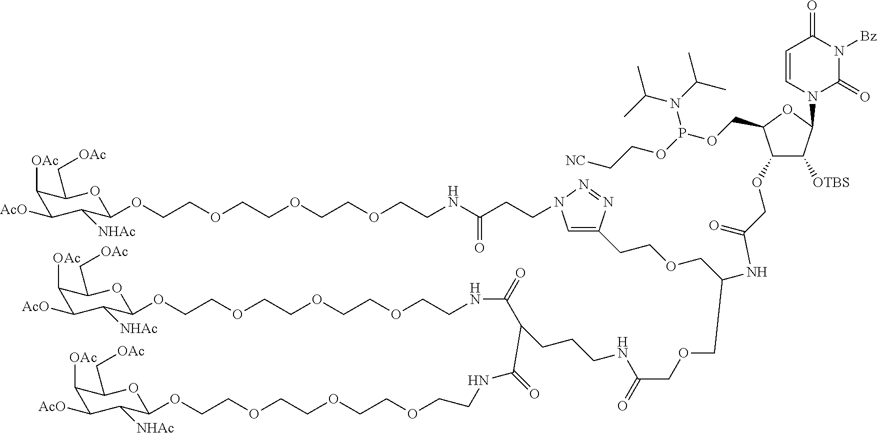

or a salt, solvate, or hydrate thereof. In some embodiments, the compound is:

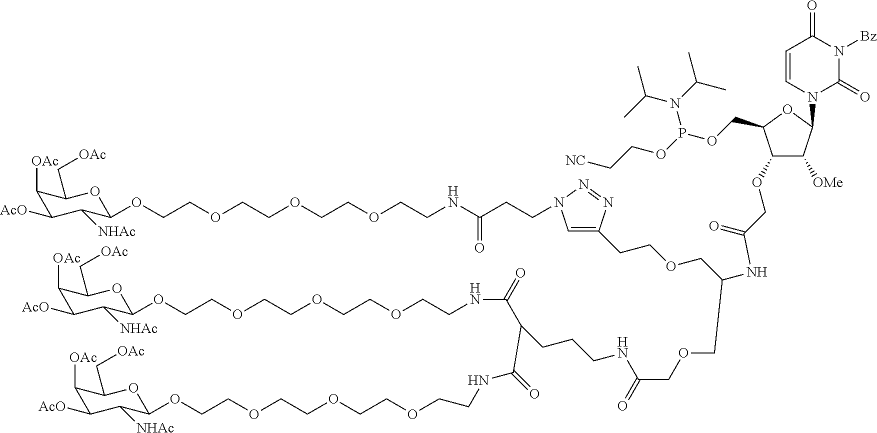

or a salt, solvate, or hydrate thereof. In some embodiments, the compound is:

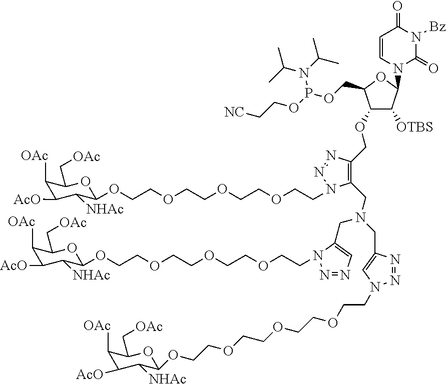

or a salt, solvate, or hydrate thereof. In some embodiments, the compound is:

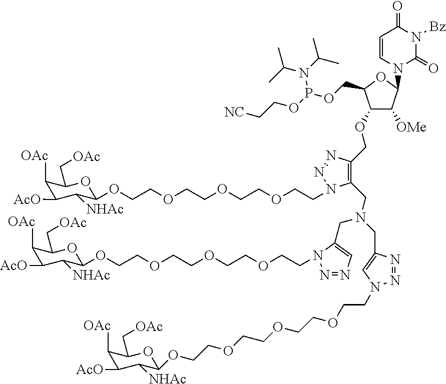

or a salt, solvate, or hydrate thereof. In some embodiments, the compound is:

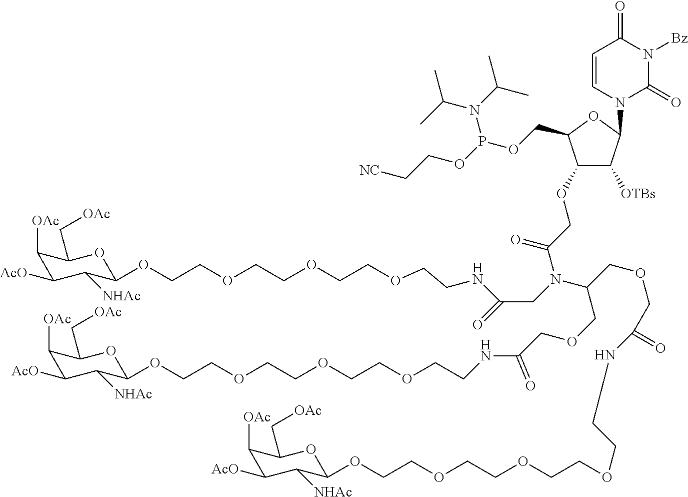

or a salt, solvate, or hydrate thereof. In some embodiments, the compound is:

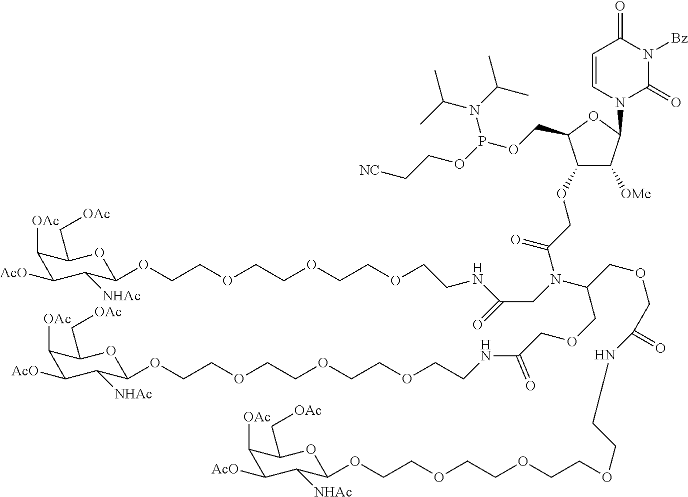

or a salt, solvate, or hydrate thereof.

In another aspect, compounds of Formula (XXIV), or a salt, solvate, or hydrate thereof, are provided:

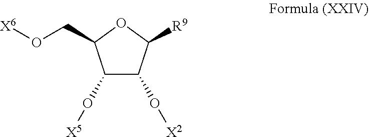

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, or optionally substituted heteroaryl;
X² is H, alkyl, or Protecting Group (PG);
X⁵ is H, Protecting Group (PG), or

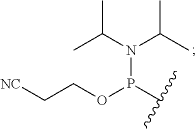 [SMILES: *P(OCCC#N)N(C(C)C)C(C)C]

and
X⁶ is

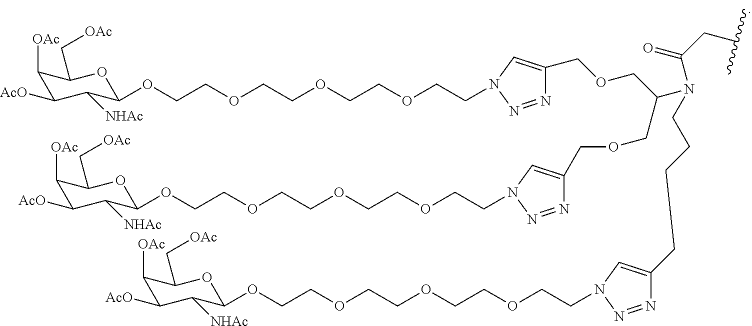 [SMILES: *CC(=O)N(CCCCc1cn(CCOCCOCCOCCOC2OC(COC(C)=O)C(OC(C)=O)C(OC(C)=O)C2NC(C)=O)nn1)C(COCc1cn(CCOCCOCCOCCOC2OC(COC(C)=O)C(OC(C)=O)C(OC(C)=O)C2NC(C)=O)nn1)COCc1cn(CCOCCOCCOCCOC2OC(COC(C)=O)C(OC(C)=O)C(OC(C)=O)C2NC(C)=O)nn1]

In another aspect, compounds of Formula (XXV), or a salt, solvate, or hydrate thereof, are provided:

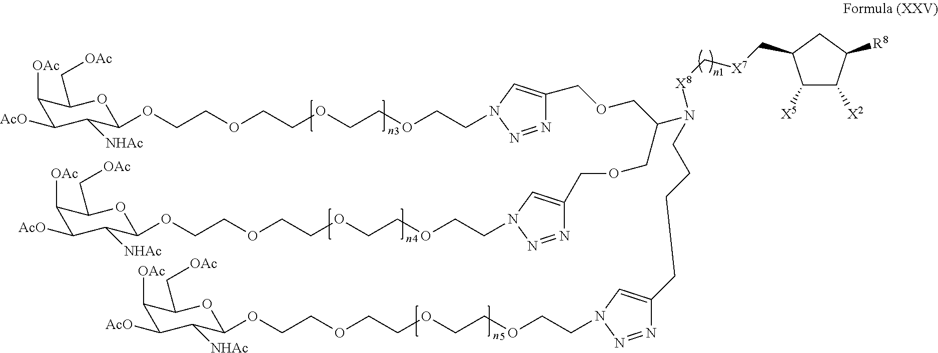

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, or optionally substituted heteroaryl;
X² is H, OH, O-alkyl, alkyl, heteroalkyl, halogen, or O-Protecting Group (PG);
X⁵ is H, OH, O-Protecting Group (PG), or

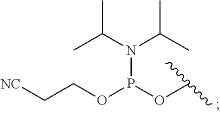 [SMILES: *OP(OCCC#N)N(C(C)C)C(C)C]

X⁷ is O;
X⁸ is CO, SO₂, P(O)O, CH₂—O—C(O), CH₂—NH—C(O), CH₂—NH—SO₂, or CH₂; and
each n₁, n₃, n₄ and n₅ is independently 0, 1, 2, 3, 4, 5, or 6.

In some embodiments, the compound is:

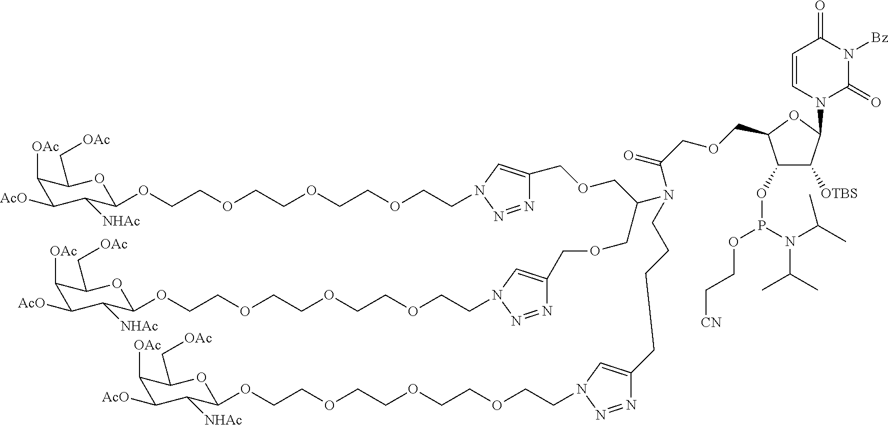

or a salt, solvate, or hydrate thereof. In some embodiments, the compound is:

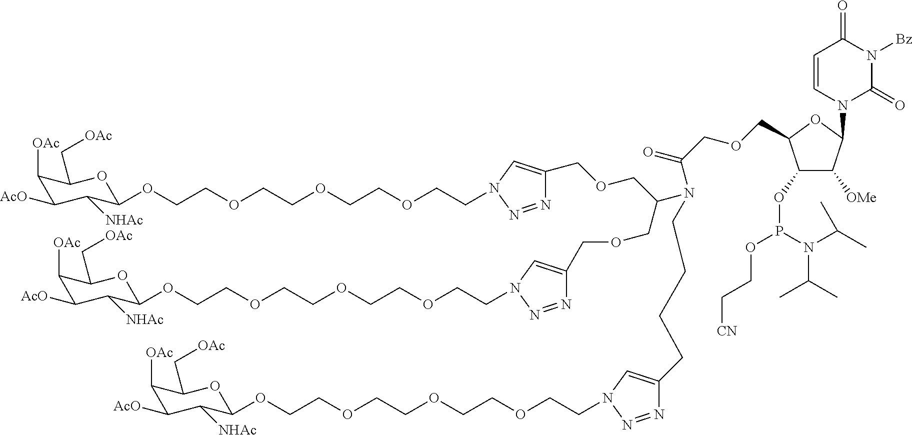

or a salt, solvate, or hydrate thereof. In some embodiments, any of the compounds provided herein may be conjugated to an oligonucleotide. In some embodiments, any of the compounds provided herein may be deprotected (e.g., the acetyl groups on the oxygen atoms in GalNAc may be removed) and may be conjugated to an oligonucleotide. In some embodiments, any of the compounds provided herein, or an oligonucleotide comprising any of the compounds provided herein, may be provided in a composition comprising a pharmaceutically acceptable carrier. In some embodiments, any of the compounds, oligonucleotides, or compositions provided herein may be administered to a subject in a method for modulating protein function in a subject. In some embodiments, any of the compounds, oligonucleotides, or compositions provided herein may be administered to a subject in a method for treating or ameliorating a disease, disorder, or symptom thereof in a subject. In some embodiments, the disease, disorder, or symptom thereof is a liver disease, disorder, or symptom thereof.

In another aspect, the present disclosure provides methods for making any of the compounds provided herein comprising one or more compounds and chemical transformations described herein, including the examples herein, e.g., Examples 1-23.

In another aspect, the present disclosure provides compounds of Formula (XXVI), or a salt, solvate, or hydrate thereof:

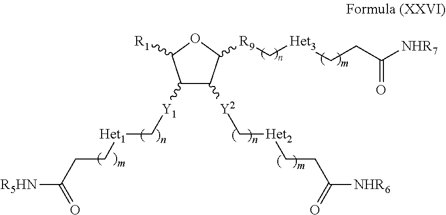

wherein:
each n is independently 1, 2, 3, 4, or 5;
each m is independently 0, 1, 2, 3, 4, 5, or 6;
each o is independently 0, 1, 2, 3, 4, 5, or 6;
each Y₁ is independently O, CH(R^{a}), S, S(═O), S(═O)₂, NH, substituted N group, NHC(═O), C(═O)NH, P(═O)(OH)—O—, P(═O)(SH)—O, P(═S)(SH)—O, —O—P(═O)(OH)—O—, —O—P(═O)(SH)—O—, —O—P(═S)(SH)—O—, —O—P(═O)(OH)—, —O—P(O)(SH)—, —O—P(═S)(SH)—;
each Y₂ is independently O, CH(R^{b}), S, S(═O), S(═O)₂, NH, substituted N group, NHC(═O), C(═O)NH, P(═O)(OH)—O—, P(═O)(SH)—O, P(═S)(SH)—O, —O—P(═O)(OH)—O—, —O—P(═O)(SH)—O—, —O—P(═S)(SH)—O—, —O—P(═O)(OH)—, —O—P(═O)(SH)—, —O—P(═S)(SH)—;
each of Het₁, Het₂, and Het₃ is independently optionally substituted heteroaryl or optionally substituted heterocyclyl;
each R₁ is independently alkyl-O-phosphoramidite, optionally substituted alkenyl phosphoramidite, optionally substituted alkynyl phosphoramidite, OH, NH₂, NHR^{a}, N₃, C(═O)OH, C(═O)X, CN, SH, SSH, SO₂X, C(═O)NHNH₂, NHNH₂, C(═S)NHNH₂, C(═S)NH₂, NHOH, C(═O)CH₂X, malonyl, alkyl, alkenyl, dienyl, alkynyl, heteroalkyl, —OP(═S)X, —C(═O)H, —C(═O)R^{a}, —N═C═O, —N═C═NR^{a}, —N═C═S, CHX, —OP(═O)OH, phosphane, alkoxyphosphane, —C(R^{a})₂, a Michael acceptor, a protein, or a therapeutic agent for modulating hepatocytes or treating liver disease, wherein X is a leaving group;
each R₅, R₆, and R₇ is independently

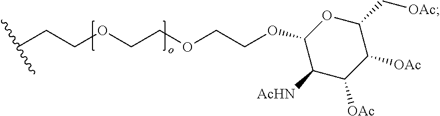

R₉ is optionally substituted heterocyclyl;
each R^{a} is independently H, alkyl, halo, OR^{c}, or SR^{c};
each R^{b} is independently H, alkyl, halo, OR^{c}, or SR^{c}; and
each R^{c} is independently H or alkyl;
as well as compositions comprising, methods of treatment comprising, and methods of making comprising, such compounds, and salts, solvates, and hydrates thereof.

In one aspect of the invention each R₁ is independently alkyl-O-phosphoramidite, optionally substituted alkenyl phosphoramidite, or optionally substituted alkynyl phosphoramidite.

In one aspect of the invention each R₁ is independently OH, NH₂, NHR^{a}, N₃, C(═O)OH, C(═O)X, CN, SH, SSH, SO₂X, C(═O)NHNH₂, NHNH₂, C(═S)NHNH₂, C(═S)NH₂, NHOH, C(═O)CH₂X, malonyl, alkyl, alkenyl, dienyl, alkynyl, heteroalkyl, —OP(═S)X, —C(═O)H, —C(═O)R^{a}, —N═C═O, —N═C═NR^{a}, —N═C═S, CHX, —OP(═O)OH, phosphane, alkoxyphosphane, or —C(R^{a})₂, wherein X is a leaving group.

In one aspect of the invention each R₁ is independently a Michael acceptor, a protein, or a therapeutic agent for modulating hepatocytes or treating liver disease.

In another aspect, compounds of Formula (XXVI-a), or a salt, solvate, or hydrate thereof, are provided:

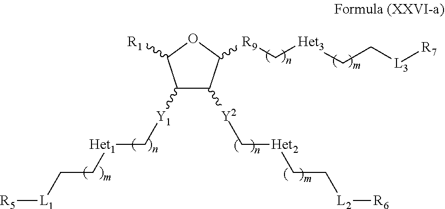

wherein:
each n is independently 1, 2, 3, 4, or 5;
each m is independently 0, 1, 2, 3, 4, 5, or 6;
each o is independently 0, 1, 2, 3, 4, 5, or 6;
each of L₁, L₂, and L₃ is independently absent, C(═O), or C(═O)NH;
each Y₁ is independently O, CH(R^{a}), S, S(═O), S(═O)₂, NH, substituted N group, NHC(═O), C(═O)NH, P(═O)(OH)—O—, P(═O)(SH)—O, P(═S)(SH)—O, —O—P(═O)(OH)—O—, —O—P(═O)(SH)—O—, —O—P(═S)(SH)—O—, —O—P(═O)(OH)—, —O—P(═O)(SH)—, —O—P(═S)(SH)—;
each Y₂ is independently O, CH(R^{b}), S, S(═O), S(═O)₂, NH, substituted N group, NHC(═O), C(═O)NH, P(═O)(OH)—O—, P(═O)(SH)—O, P(═S)(SH)—O, —O—P(═O)(OH)—O—, —O—P(═O)(SH)—O—, —O—P(═S)(SH)—O—, —O—P(═O)(OH)—, —O—P(═O)(SH)—, —O—P(═S)(SH)—;
each of Het₁, Het₂, and Het₃ is independently optionally substituted heteroaryl or optionally substituted heterocyclyl;
each R₁ is independently alkyl-O-phosphoramidite, optionally substituted alkenyl phosphoramidite, optionally substituted alkynyl phosphoramidite, OH, NH₂, NHR^{a}, N₃, CH₂N₃, C(═O)OH, C(═O)X, CN, SH, SSH, SO₂X, C(═O)NHNH₂, NHNH₂, C(═S)NHNH₂, C(═S)NH₂, NHOH, C(═O)CH₂X, malonyl, alkyl, alkenyl, dienyl, alkynyl, heteroalkyl, —OP(═S)X, —C(═O)H, —C(═O)R^{a}, —N═C═O, —N═C═S, CHX, —OP(═O)OH, phosphate, phosphane, alkoxyphosphane, —C(R^{a})₂, a Michael acceptor, a protein, or a therapeutic agent for modulating hepatocytes or treating liver disease, wherein X is a leaving group; or R¹ comprises an oligonucleotide linked by a bond, a phosphodiester bond, a phosphorothioate bond, a triazole, a tetrazole, an amide, a reverse-amide, a carbamate, a carbonate, urea, alkyl, or heteroalkyl;
each R₅, R₆, and R₇ is independently

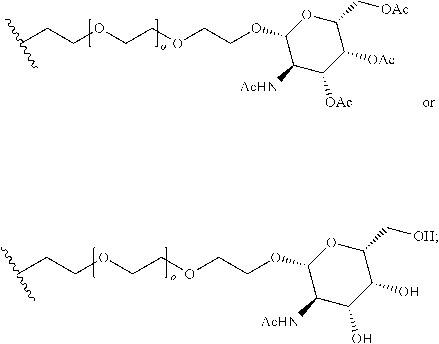

R₉ is optionally substituted heterocyclyl;
each R^{a} is independently H, alkyl, halo, OR^{c}, or SR^{c};
each R^{b} is independently H, alkyl, halo, OR^{c}, or SR^{c}; and
each R^{c} is independently H or alkyl;
as well as compositions comprising, methods of treatment comprising, and methods of making comprising, such compounds, and salts, solvates, and hydrates thereof.

In some embodiments, R₉ is an optionally substituted nitrogen-containing heterocyclyl. In some embodiments, R₉ is an optionally substituted pyrimidinyl. In some embodiments, R₉ is a 4H-1λ²,3λ²-pyrimidine-2,4-dione. In certain embodiments, R₉ is

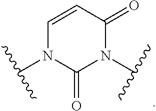 [SMILES: *n1ccc(=O)n(*)c1=O]

In some embodiments, each of Het₁, Het₂, and Het₃ is independently optionally substituted nitrogen-containing heterocyclyl or optionally substituted nitrogen-containing heteroaryl. In some embodiments, each of Het₁, Het₂, and Het₃ is independently optionally substituted 1, 2, 3-triazolyl. In some embodiments, each of Het₁, Het₂, and Het₃ is independently optionally substituted 1λ²,2,3-triazol-4-yl. In certain embodiments, each of Het₁, Het₂, and Het₃ is independently

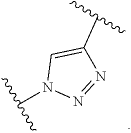 [SMILES: *c1cn(*)nn1]

In some embodiments, two of R₅, R₆, and R₇ are each the same. In some embodiments, R₅, and R₆ are each the same. In some embodiments, three of R₅, R₆, and R₇ are each the same. In some embodiments, o is the same in each of R₅, R₆, and R₇. In certain embodiments, o is the same in each of R₅ and R₆. In certain embodiments, all three of R₅, R₆, and R₇ are

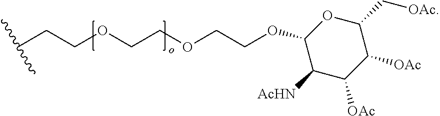

In certain embodiments, all three of R₅, R₆, and R₇ are

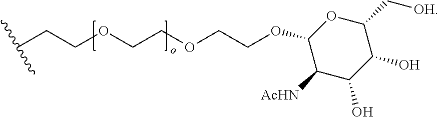

In some embodiments of the invention, each R₁ is independently alkyl-O-phosphoramidite, optionally substituted alkenyl phosphoramidite, or optionally substituted alkynyl phosphoramidite. In some embodiments of the invention, each R₁ is independently OH, NH₂, NHR^{a}, N₃, C(═O)OH, C(═O)X, CN, SH, SSH, SO₂X, C(═O)NHNH₂, NHNH₂, C(═S)NHNH₂, C(═S)NH₂, NHOH, C(═O)CH₂X, malonyl, alkyl, alkenyl, dienyl, alkynyl, heteroalkyl, —OP(═S)X, —C(═O)H, —C(═O)R^{a}, —N═C═O, —N═C═NR^{a}, —N═C═S, CHX, —OP(═O)OH, phosphane, alkoxyphosphane, or —C(R^{a})₂, wherein X is a leaving group. In some embodiments of the invention, each R₁ is independently a Michael acceptor, a protein, or a therapeutic agent for modulating hepatocytes or treating liver disease. In certain embodiments, R₁ is

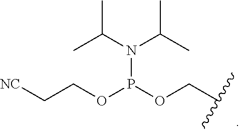 [SMILES: *COP(OCCC#N)N(C(C)C)C(C)C]

In certain embodiments, R₁ is phosphate. In certain embodiments, R₁ is CH₂N₃.

In another aspect, compounds of Formula (XXVII), or a salt, solvate, or hydrate thereof are provided:

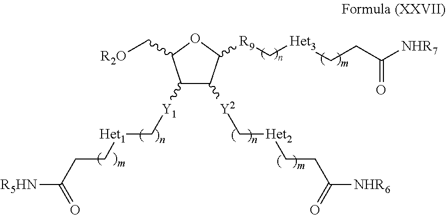

wherein,
each n is independently 1, 2, 3, 4, or 5;
each m is independently 0, 1, 2, 3, 4, 5, or 6;
each o is independently 0, 1, 2, 3, 4, 5, or 6;
each Y₁ is independently O, CH(R^{a}), S, S(═O), S(═O)₂, NH, substituted N group, NHC(═O), C(═O)NH, P(═O)(OH)—O—, P(═O)(SH)—O, P(═S)(SH)—O, —O—P(═O)(OH)—O—, —O—P(═O)(SH)—O—, —O—P(═S)(SH)—O—, —O—P(═O)(OH)—, —O—P(O)(SH)—, —O—P(═S)(SH)—;
each Y₂ is independently O, CH(R^{b}), S, S(═O), S(═O)₂, NH, substituted N group, NHC(═O), C(═O)NH, P(═O)(OH)—O—, P(═O)(SH)—O, P(═S)(SH)—O, —O—P(═O)(OH)—O—, —O—P(═O)(SH)—O—, —O—P(═S)(SH)—O—, —O—P(═O)(OH)—, —O—P(═O)(SH)—, —O—P(═S)(SH)—;
each of Het₁, Het₂, and Het₃ is independently optionally substituted heteroaryl or optionally substituted heterocyclyl;
each R₅, R₆, and R₇ is independently

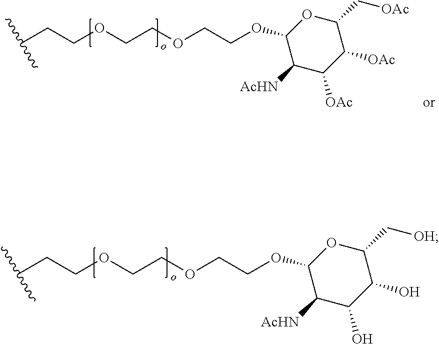

R₉ is optionally substituted heterocyclyl; and
R² is H, Protecting Group (PG),

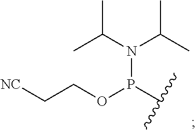 [SMILES: *P(OCCC#N)N(C(C)C)C(C)C]

each R^{a} is independently H, alkyl, halo, OR^{c}, or SR^{c};
each R^{b} is independently H, alkyl, halo, OR^{c}, or SR^{c}; and
each R^{c} is independently H or alkyl.

In some embodiments, R² is H. In some embodiments, R² is Protecting Group (PG). In certain embodiments, R² is

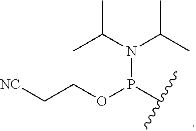 [SMILES: *P(OCCC#N)N(C(C)C)C(C)C]

In some embodiments, R₉ is an optionally substituted nitrogen-containing heterocyclyl. In some embodiments, R₉ is an optionally substituted dihydropyrimidinyl, optionally substituted tetrahydropyrimidinyl, or optionally substituted hexahydropyrimidinyl. In some embodiments, R₉ is a 4H-1λ²,3λ²-pyrimidine-2,4-dione. In certain embodiments, R₉ is

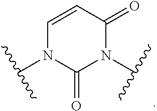 [SMILES: *n1ccc(=O)n(*)c1=O]

In some embodiments, each of Het₁, Het₂, and Het₃ is independently optionally substituted nitrogen-containing heterocyclyl or optionally substituted nitrogen-containing heteroaryl. In some embodiments, each of Het₁, Het₂, and Het₃ is independently optionally substituted 1, 2, 3-triazolyl. In some embodiments, each of Het₁, Het₂, and Het₃ is independently optionally substituted 1λ²,2,3-triazol-4-yl. In certain embodiments, each of Het₁, Het₂, and Het₃ is independently

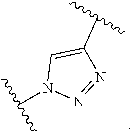 [SMILES: *c1cn(*)nn1]

In some embodiments, two of R₅, R₆, and R₇ are each the same. In some embodiments, R₅, and R₆ are each the same. In some embodiments, three of R₅, R₆, and R₇ are each the same. In some embodiments, o is the same in each of R₅, R₆, and R₇. In certain embodiments, o is the same in each of R₅ and R₆.

In some embodiments, the compound is of Formula (XXVII-a):

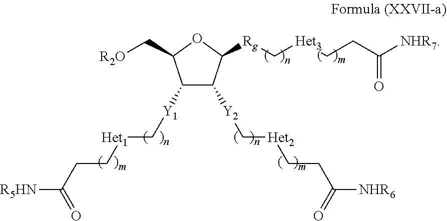

In some embodiments, the compound is of the formula:

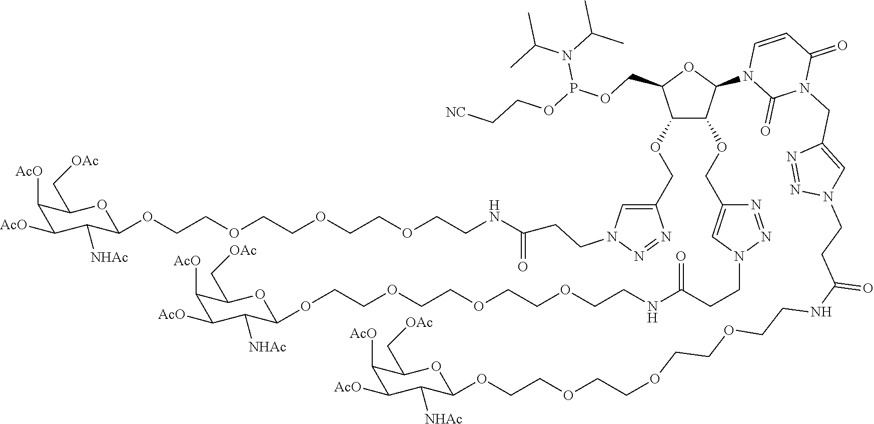 [SMILES: CC(=O)NC1C(OCCOCCOCCOCCNC(=O)CCn2cc(CO[C@@H]3[C@H](OCc4cn(CCC(=O)NCCOCCOCCOCCOC5OC(COC(C)=O)C(OC(C)=O)C(OC(C)=O)C5NC(C)=O)nn4)[C@@H](COP(OCCC#N)N(C(C)C)C(C)C)O[C@H]3n3ccc(=O)n(Cc4cn(CCC(=O)NCCOCCOCCOCCOC5OC(COC(C)=O)C(OC(C)=O)C(OC(C)=O)C5NC(C)=O)nn4)c3=O)nn2)OC(COC(C)=O)C(OC(C)=O)C1OC(C)=O]

or a salt, solvate, or hydrate thereof. In some embodiments, the compound is of the formula:

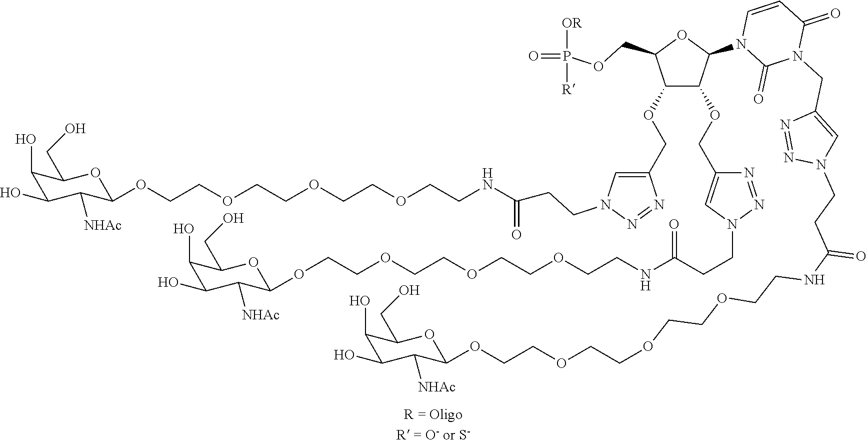

or a salt, solvate, or hydrate thereof. In some embodiments, the compound is of the formula.

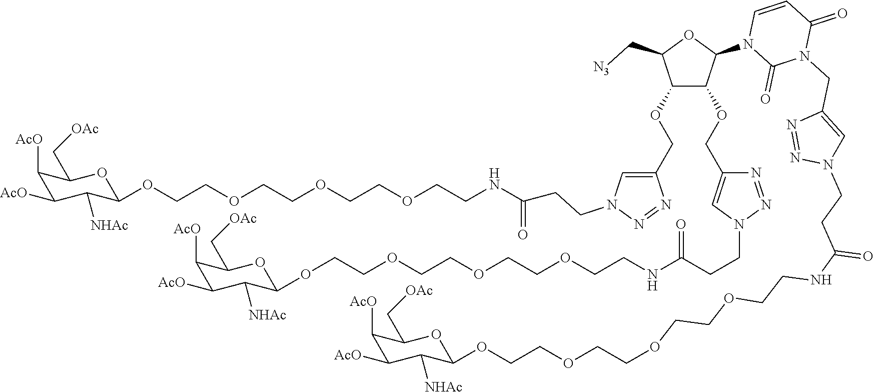 [SMILES: CC(=O)NC1C(OCCOCCOCCOCCNC(=O)CCn2cc(CO[C@@H]3[C@H](OCc4cn(CCC(=O)NCCOCCOCCOCCOC5OC(COC(C)=O)C(OC(C)=O)C(OC(C)=O)C5NC(C)=O)nn4)[C@@H](CN=[N+]=[N-])O[C@H]3n3ccc(=O)n(Cc4cn(CCC(=O)NCCOCCOCCOCCOC5OC(COC(C)=O)C(OC(C)=O)C(OC(C)=O)C5NC(C)=O)nn4)c3=O)nn2)OC(COC(C)=O)C(OC(C)=O)C1OC(C)=O]

or a salt, solvate, or hydrate thereof. In some embodiments, the compound is of the formula:

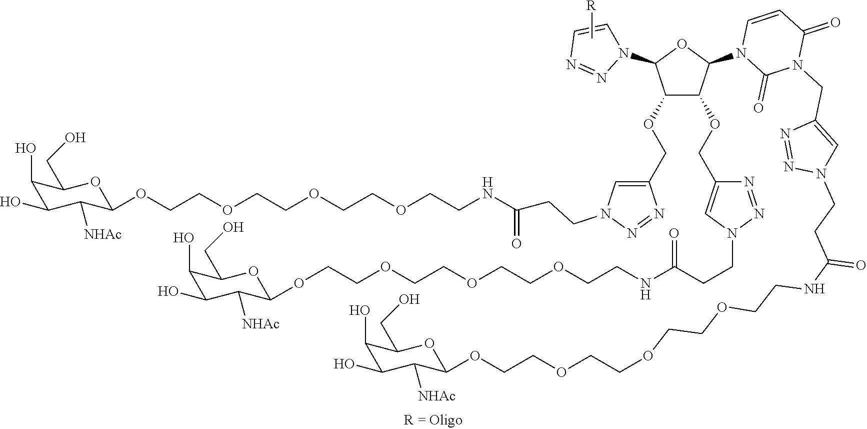

or a salt, solvate, or hydrate thereof. In some embodiments, the compound is of the formula:

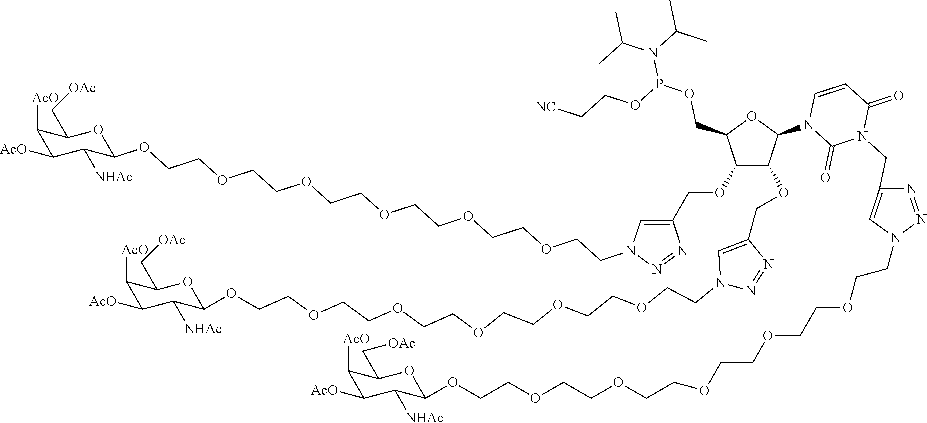 [SMILES: CC(=O)NC1C(OCCOCCOCCOCCOCCOCCn2cc(CO[C@@H]3[C@H](OCc4cn(CCOCCOCCOCCOCCOCCOC5OC(COC(C)=O)C(OC(C)=O)C(OC(C)=O)C5NC(C)=O)nn4)[C@@H](COP(OCCC#N)N(C(C)C)C(C)C)O[C@H]3n3ccc(=O)n(Cc4cn(CCOCCOCCOCCOCCOCCOC5OC(COC(C)=O)C(OC(C)=O)C(OC(C)=O)C5NC(C)=O)nn4)c3=O)nn2)OC(COC(C)=O)C(OC(C)=O)C1OC(C)=O]

or a salt, solvate, or hydrate thereof. In some embodiments, the compound is of the formula:

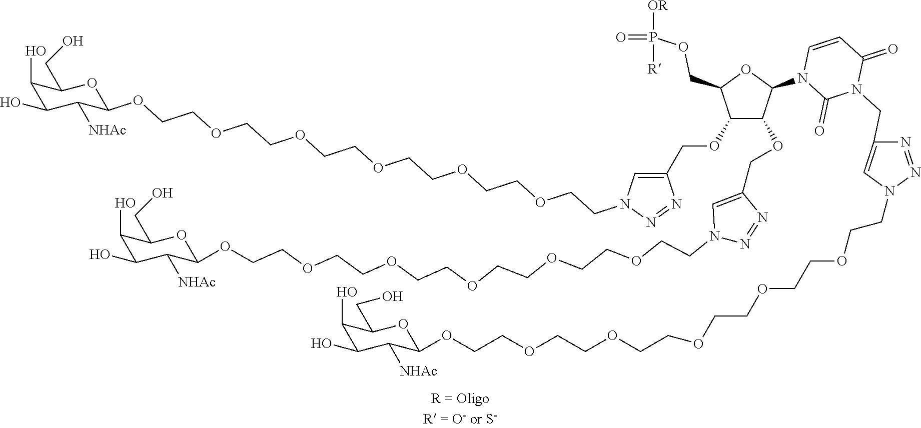

or a salt, solvate, or hydrate thereof.

In another aspect, the present disclosure provides modified oligonucleotides comprising a moiety of Formula (XXVIII), or a salt, solvate, or hydrate thereof:

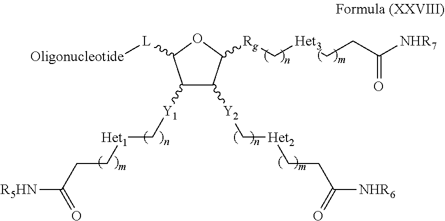

wherein:
each n is independently 0, 1, 2, 3, 4, or 5;
each m is independently 0, 1, 2, 3, 4, 5, or 6;
each o is independently 0, 1, 2, 3, 4, 5, or 6;
each Y₁ is independently O, CH(R^{a}), S, S(═O), S(═O)₂, NH, substituted N group, NHC(═O), C(═O)NH, P(═O)(OH)—O—, P(═O)(SH)—O, P(═S)(SH)—O, —O—P(═O)(OH)—O—, —O—P(═O)(SH)—O—, —O—P(═S)(SH)—O—, —O—P(═O)(OH)—, —O—P(═O)(SH)—, —O—P(═S)(SH)—;
each Y₂ is independently O, CH(R^{b}), S, S(═O), S(═O)₂, NH, substituted N group, NHC(═O), C(═O)NH, P(═O)(OH)—O—, P(═O)(SH)—O, P(═S)(SH)—O, —O—P(═O)(OH)—O—, —O—P(═O)(SH)—O—, —O—P(═S)(SH)—O—, —O—P(═O)(OH)—, —O—P(O)(SH)—, —O—P(═S)(SH)—;
each of Het₁, Het₂, and Het₃ is independently optionally substituted heteroaryl or optionally substituted heterocyclyl;
each R₅, R₆, and R₇ is independently

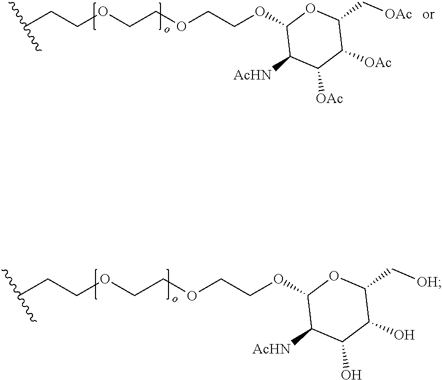

and
R⁹ is optionally substituted heterocyclyl;
L is bond, O, CH₂, S, S(═O), S(═O)₂, NH, substituted N group, NHC(═O), C(═O)NH, alkyl, alkenyl, dienyl, alkynyl, heteroalkyl, phosphate, or a thiol-Michael adduct;
each R^{a} is independently H, alkyl, halo, OR^{c}, or SR^{c};
each R^{b} is independently H, alkyl, halo, OR^{c}, or SR^{c}; and
each R^{c} is independently H or alkyl.

In another aspect, the present disclosure provides oligonucleotides comprising a moiety of Formula (XXIX), or a salt, solvate, or hydrate thereof:

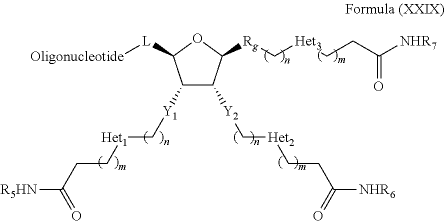

wherein:
each n is independently 0, 1, 2, 3, 4, or 5;
each m is independently 0, 1, 2, 3, 4, 5, or 6;
each o is independently 0, 1, 2, 3, 4, 5, or 6;
each Y₁ is independently O, CH(R^{a}), S, S(═O), S(═O)₂, NH, substituted N group, NHC(═O), C(═O)NH, P(═O)(OH)—O—, P(═O)(SH)—O, P(═S)(SH)—O, —O—P(═O)(OH)—O—, —O—P(═O)(SH)—O—, —O—P(═S)(SH)—O—, —O—P(═O)(OH)—, —O—P(O)(SH)—, —O—P(═S)(SH)—;
each Y₂ is independently O, CH(R^{b}), S, S(═O), S(═O)₂, NH, substituted N group, NHC(═O), C(═O)NH, P(═O)(OH)—O—, P(═O)(SH)—O, P(═S)(SH)—O, —O—P(═O)(OH)—O—, —O—P(═O)(SH)—O—, —O—P(═S)(SH)—O—, —O—P(═O)(OH)—, —O—P(═O)(SH)—, —O—P(═S)(SH)—;
each of Het₁, Het₂, and Het₃ is independently optionally substituted heteroaryl or optionally substituted heterocyclyl;
each R₅, R₆, and R₇ is independently

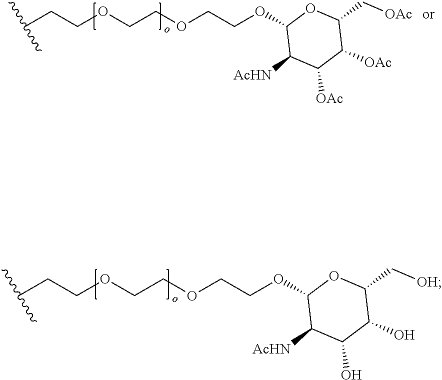

R₉ is optionally substituted heterocyclyl;
L is bond, O, CH₂, S, S(═O), S(═O)₂, NH, substituted N group, NHC(═O), C(═O)NH, alkyl, alkenyl, dienyl, alkynyl, heteroalkyl, phosphate, or a thiol-Michael adduct;
each R^{a} is independently H, alkyl, halo, OR^{c}, or SR^{c};
each R^{b} is independently H, alkyl, halo, OR^{c}, or SR^{c}; and
each R^{c} is independently H or alkyl.

In some embodiments, the oligonucleotide comprises an siRNA, an miRNA, an ADAR recruiting molecule, an ADAR targeting molecule, a guide RNA, or an antisense nucleic acid. In some embodiments, any of the compounds or oligonucleotides provided herein, or a salt, solvate or hydrate thereof, may be used as a reagent in a chemical reaction.

Also provided herein are compositions comprising any of the compounds provided herein, and a pharmaceutically acceptable carrier.

Further provided herein are methods of making any of the compounds disclosed herein comprising one or more of the compounds and chemical transformations described herein, including the examples herein, e.g., Examples 1-23.

In some embodiments, the compound is of Formula (XXXI), or a salt, solvate, or hydrate thereof:

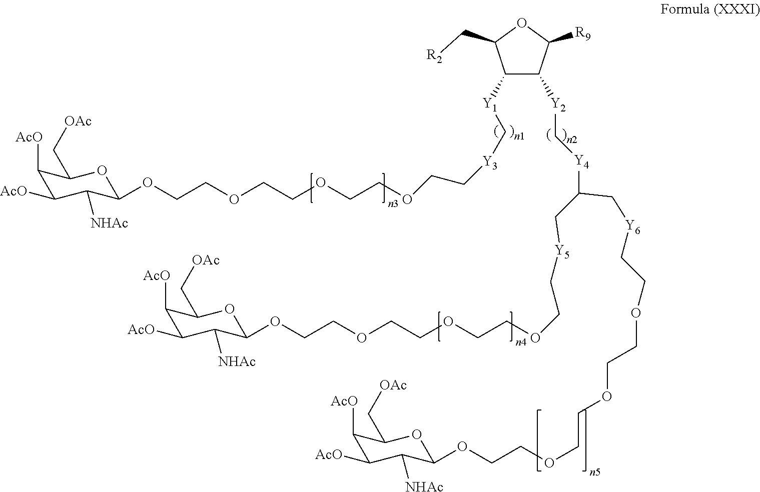

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, optionally substituted heteroaryl, optionally substituted carbocyclyl, optionally substituted heterocyclyl, or a nucleobase isostere;
R² is H, O-Protecting Group (PG),

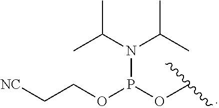 [SMILES: *OP(OCCC#N)N(C(C)C)C(C)C]

a linker, an aide, a carboxylic acid, or an amine;
Y₁ is O, CH₂, CH₂O, or optionally substituted NH;
Y₂ is O, CH₂, CH₂O, or optionally substituted NH;
Y₃ is CO, C(O)NH, C(O)—NH—CH₂—C(O)—NH, SO₂, P(O)O, CH₂—O—C(O), CH₂—NH—C(O), CH₂—NH—SO₂, or CH₂;
Y₄ is CO, C(O)NH, SO₂, P(O)O, CH₂—O—C(O), CH₂—NH—C(O), CH₂—NH—SO₂, or CH₂;
Y₅ is CO, C(O)NH, OC(O)NH, or OCH₂C(O)NH;
Y₆ is CO, C(O)NH, OC(O)NH, or OCH₂C(O)NH;
n₂ is 0, 1, 2, 3, 4, 5, or 6;
each n₁, n₃, n₄ and n₅ is independently 0, 1, 2, 3, 4, 5, 6, 7, 8, 9, or 10.

In some embodiments, the compound is of formula:

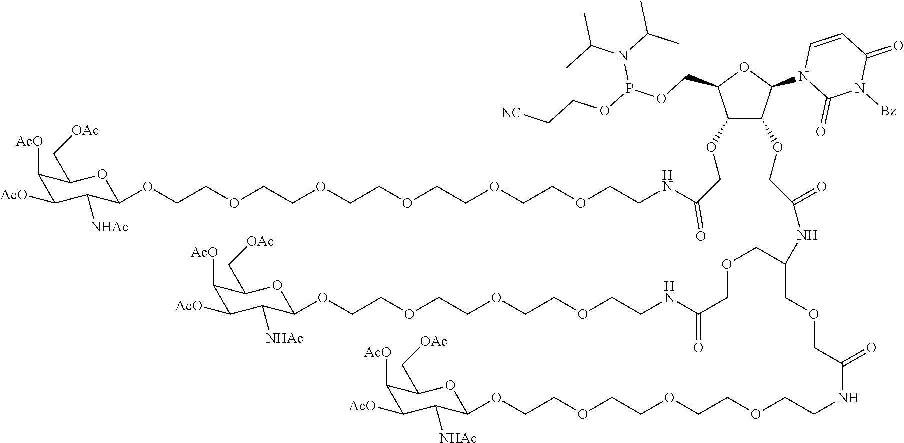

or a salt, solvate, or hydrate thereof. In some embodiments, the compound is of formula:

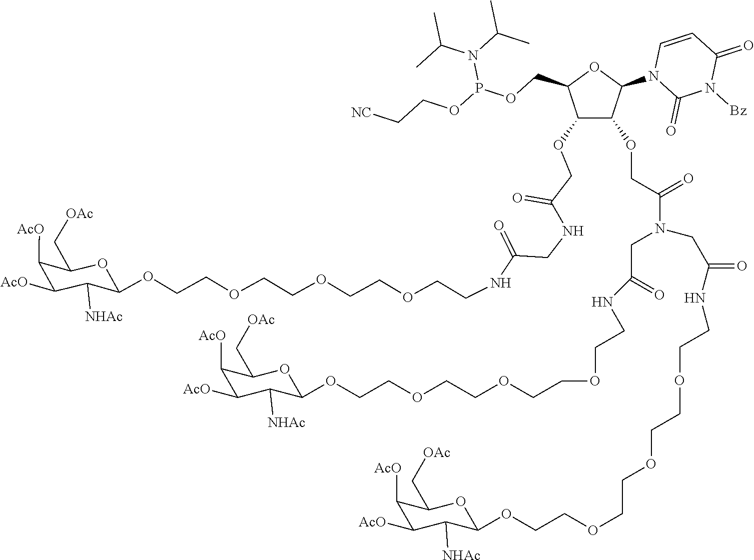

or a salt, solvate, or hydrate thereof.

In some embodiments, the compound is of Formula (XXXII), or a salt, solvate, or hydrate thereof:

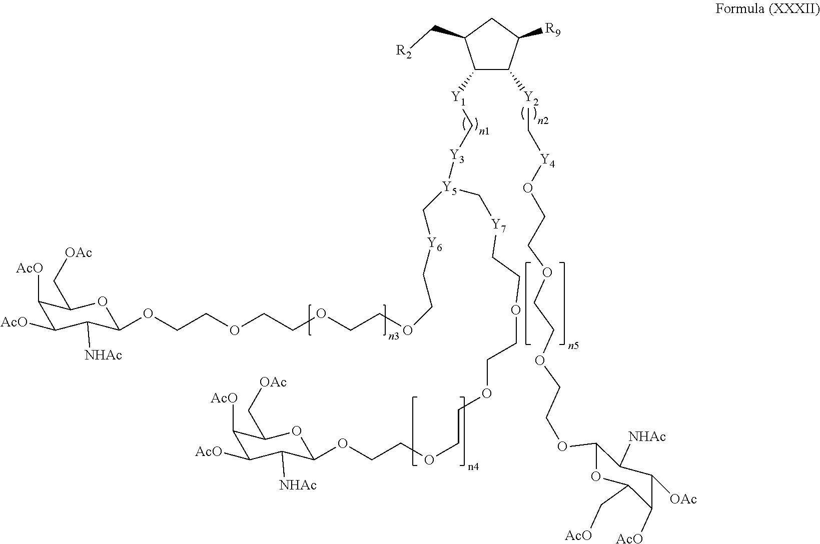

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, optionally substituted heteroaryl, optionally substituted carbocyclyl, optionally substituted heterocyclyl, or a nucleobase isostere;
R² is H, O-Protecting Group (PG), or

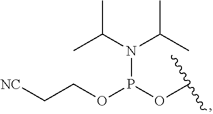 [SMILES: *OP(OCCC#N)N(C(C)C)C(C)C]

a linker, an azide, a carboxylic acid, or an amine;
Y₁ is O, CH₂, CH₂O, or optionally substituted NH;
Y₂ is O, CH₂, CH₂O, or optionally substituted NH;
Y₃ is CO, C(O)NH, C(O)—NH—CH₂—C(O)—NH, SO₂, P(O)O, CH₂—O—C(O), CH₂—NH—C(O), CH₂—NH—SO₂, or CH₂;
Y₄ is CO, C(O)NH, C(O)—NH—CH₂—C(O)—NH, SO₂, P(O)O, CH₂—O—C(O), CH₂—NH—C(O), CH₂—NH—SO₂, or CH₂;
Y₅ is CO, C(O)NH, OC(O)NH, or OCH₂C(O)NH;
Y₆ is CO, C(O)NH, OC(O)NH, or OCH₂C(O)NH;
Y₇ is CO, C(O)NH, OC(O)NH, or OCH₂C(O)NH;
n₂ is 0, 1, 2, 3, 4, 5, or 6;
each n₁, n₃, n₄ and n₅ is independently 0, 1, 2, 3, 4, 5, 6, 7, 8, 9, or 10.

In some embodiments, the compound is of the formula:

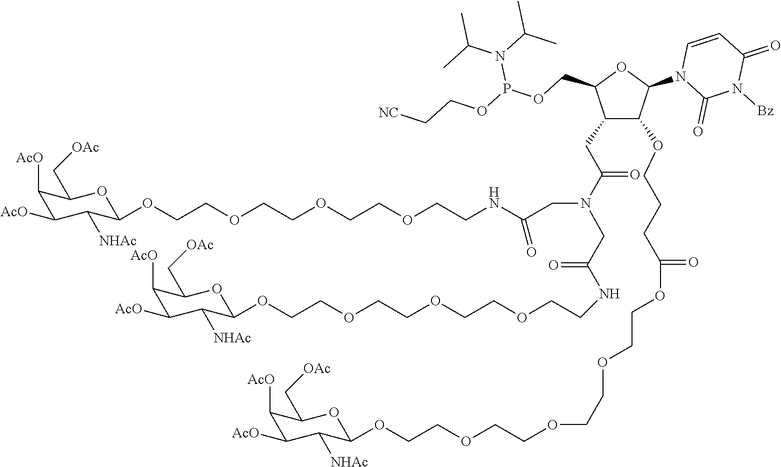

In some embodiments, the compound is of Formula (XXXII-a), or a salt, solvate, or hydrate thereof:

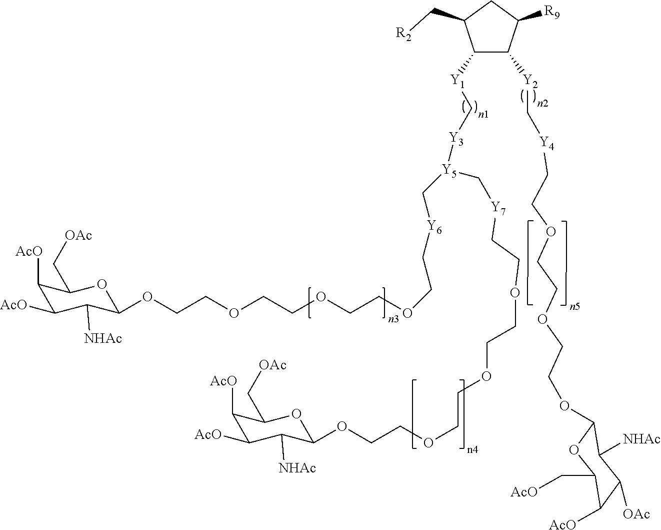

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, optionally substituted heteroaryl, optionally substituted carbocyclyl, optionally substituted heterocyclyl, or a nucleobase isostere;
R² is H, O-Protecting Group (PG), or

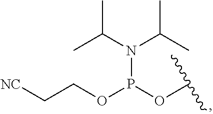 [SMILES: *OP(OCCC#N)N(C(C)C)C(C)C]

a linker, an azide, a carboxylic acid, or an amine;
Y₁ is O, CH₂, CH₂O, or optionally substituted NH;
Y₂ is O, CH₂, CH₂O, or optionally substituted NH;
Y₃ is CO, C(O)NH, C(O)—NH—CH₂—C(O)—NH, SO₂, P(O)O, CH₂—O—C(O), CH₂—NH—C(O), CH₂—NH—SO₂, or CH₂;
Y₄ is CO, C(O)NH, C(O)—NH—CH₂—C(O)—NH, SO₂, P(O)O, CH₂—O—C(O), CH₂—NH—C(O), CH₂—NH—SO₂, or CH₂;
Y₅ is CO, C(O)NH, OC(O)NH, or OCH₂C(O)NH;
Y₆ is CO, C(O)NH, OC(O)NH, or OCH₂C(O)NH;
Y₇ is CO, C(O)NH, OC(O)NH, or OCH₂C(O)NH;
n₂ is 0, 1, 2, 3, 4, 5, or 6;
each n₁, n₃, n₄ and n₅ is independently 0, 1, 2, 3, 4, 5, 6, 7, 8, 9, or 10.

In some embodiments, the compound is of the formula:

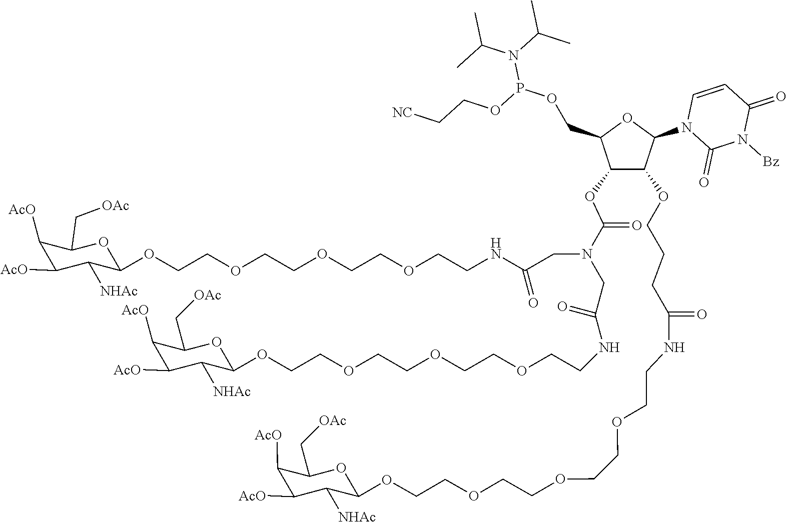

or a salt, solvate, or hydrate thereof. In some embodiments, the compound is of the formula:

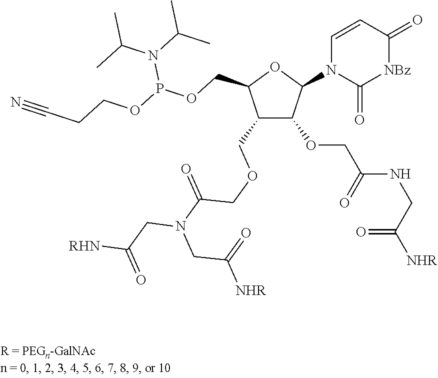

or a salt, solvate, or hydrate thereof.

In some embodiments, the compound is of Formula (XXXIII), or a salt, solvate, or hydrate thereof:

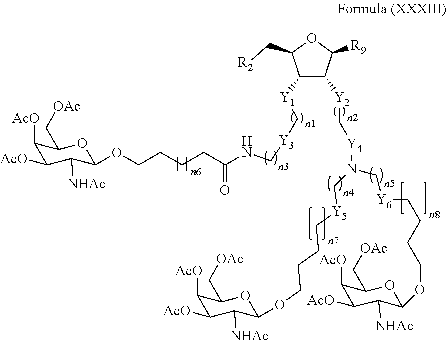

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, optionally substituted heteroaryl, optionally substituted carbocyclyl, optionally substituted heterocyclyl, or a nucleobase isostere;
R² is H, O-Protecting Group (PG), or

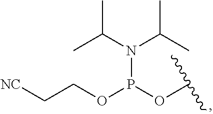 [SMILES: *OP(OCCC#N)N(C(C)C)C(C)C]

a linker, an azide, a carboxylic acid, or an amine;
Y₁ is O, CH₂, CH₂O, or optionally substituted NH;
Y₂ is O, CH₂, CH₂O, or optionally substituted NH;
Y₃ is CO, C(O)NH, C(O)—NH—CH₂—C(O)—NH, SO₂, P(O)O, CH₂—O—C(O), CH₂—NH—C(O), CH₂—NH—SO₂, or CH₂;
Y₄ is CO, C(O)NH, SO₂, P(O)O, CH₂—O—C(O), CH₂—NH—C(O), CH₂—NH—SO₂, or CH₂;
Y₅ is CO, C(O)NH, OC(O)NH, or OCH₂C(O)NH;
Y₆ is CO, C(O)NH, OC(O)NH, or OCH₂C(O)NH;
n₂ is 0, 1, 2, 3, 4, 5, or 6;
each n₁, n₃, n₄, n₅, n₆, n₇, and n₈ is independently 0, 1, 2, 3, 4, 5, 6, 7, 8, 9, or 10.

In some embodiments, the compound is of formula:

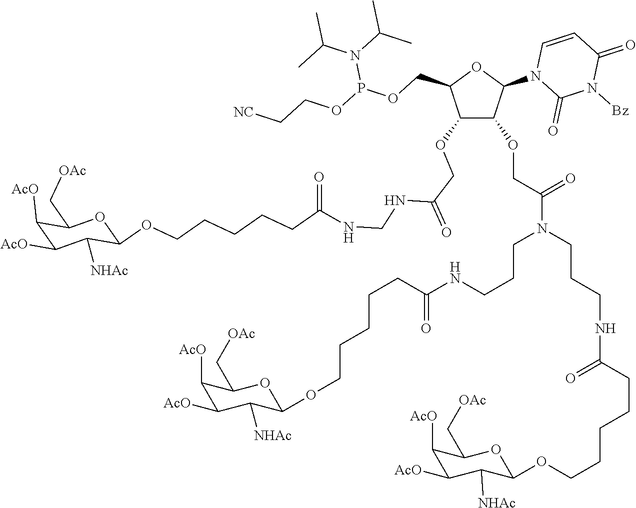

or a salt, solvate, or hydrate thereof.

In some embodiments, the compound is of Formula (XXXIV), or a salt, solvate, or hydrate thereof:

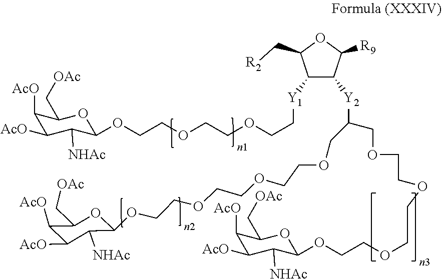

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, optionally substituted heteroaryl, optionally substituted carbocyclyl, optionally substituted heterocyclyl, or a nucleobase isostere;
R² is H, O-Protecting Group (PG),

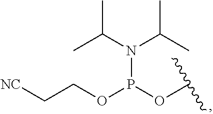 [SMILES: *OP(OCCC#N)N(C(C)C)C(C)C]

a linker, an azide, a carboxylic acid, or an amine;
Y₁ is O, CH₂, CH₂O, or optionally substituted NH;
Y₂ is O, CH₂, CH₂O, or optionally substituted NH;
Y₃ is CO, C(O)NH, C(O)—NH—CH₂—C(O)—NH, SO₂, P(O)O, CH₂—O—C(O), CH₂—NH—C(O), CH₂—NH—SO₂, or CH₂;
Y₄ is CO, C(O)NH, SO₂, P(O)O, CH₂—O—C(O), CH₂—NH—C(O), CH₂—NH—SO₂, or CH₂;
Y₅ is CO, C(O)NH, OC(O)NH, or OCH₂C(O)NH;
Y₆ is CO, C(O)NH, OC(O)NH, or OCH₂C(O)NH;
each n₁, n₂, and n₃ is independently 0, 1, 2, 3, 4, 5, 6, 7, 8, 9, or 10.

In some embodiments, the compound is of formula:

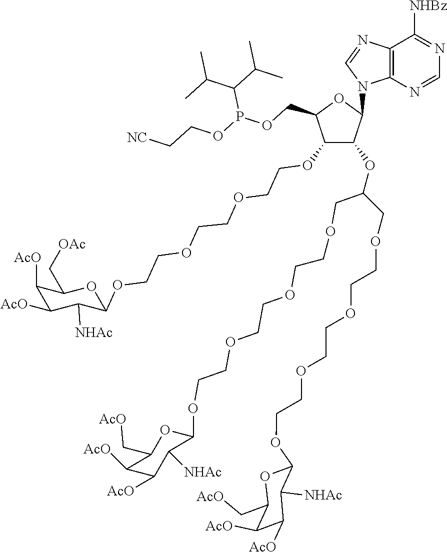

or a salt, solvate, or hydrate thereof.

In one aspect, the compounds herein are of any of the formulae delineated herein, having one or more GalNAc moieties wherein the GalNAc moieties are:

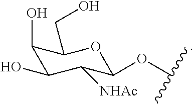 [SMILES: *OC1OC(CO)C(O)C(O)C1NC(C)=O]

In one aspect, the compounds herein are of any of the formulae delineated herein, having one or more acetylated GalNAc moieties wherein the acetylated GalNAc moieties are:

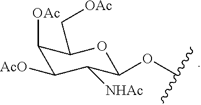 [SMILES: *OC1OC(COC(C)=O)C(OC(C)=O)C(OC(C)=O)C1NC(C)=O]

In one aspect, the compounds herein are of any of the formulae delineated herein, having one or more protected (PG) GalNAc moieties wherein the protected GalNAc moieties are:

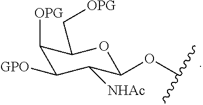

In one aspect, the oligonucleotide-containing compounds herein are of any of the formulae delineated herein, having one or more GalNAc moieties wherein the GalNAc moieties are:

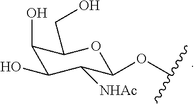 [SMILES: *OC1OC(CO)C(O)C(O)C1NC(C)=O]

In one aspect, the oligonucleotide-containing compounds herein are of any of the formulae delineated herein, having one or more acetylated GalNAc moieties wherein the acetylated GalNAc moieties are:

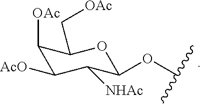 [SMILES: *OC1OC(COC(C)=O)C(OC(C)=O)C(OC(C)=O)C1NC(C)=O]

In one aspect, the oligonucleotide-containing compounds herein are of any of the formulae delineated herein, having one or more protected (PG) GalNAc moieties wherein the protected GalNAc moieties are:

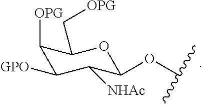

Compounds having all free hydroxy groups on the GalNAc moiety are obtained using chemical synthesis techniques known in the art (e.g., deprotection; removal of protecting group (PG)), for example by selective deacetylation of the acetyl moieties on the oxygen atoms of the GalNAc structure, while leaving intact the acetyl group attached to the nitrogen atom of the GalNAc structure.

### BRIEF DESCRIPTION OF THE DRAWINGS

FIG. 1 shows a ¹H NMR spectrum of product 2 from Example 3.

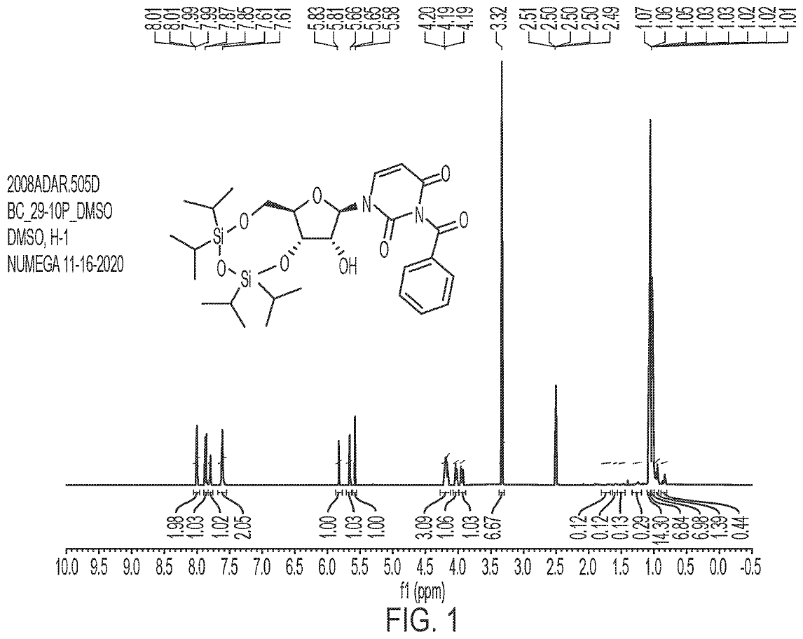

FIG. 2 shows a ¹H NMR spectrum of product 3 from Example 3.

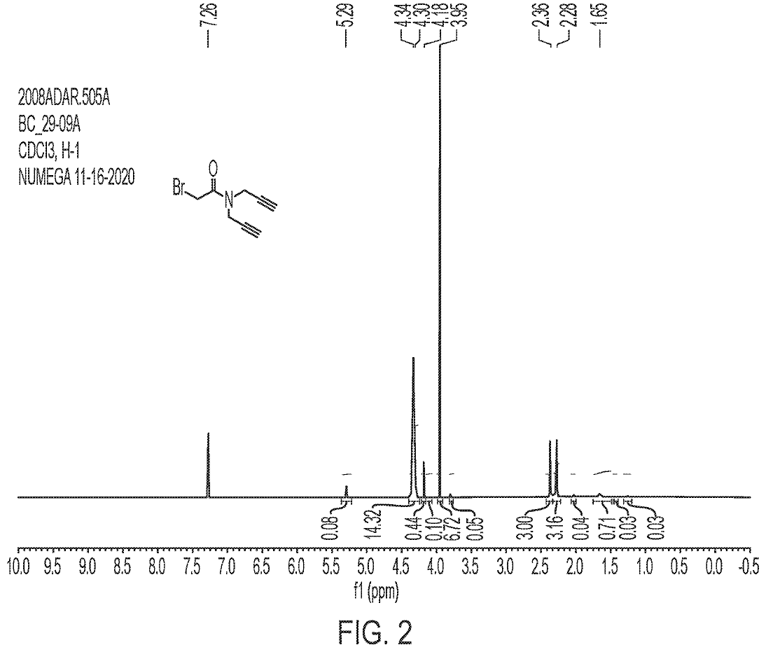

FIG. 3 shows a ¹H NMR spectrum of product 4 from Example 3.

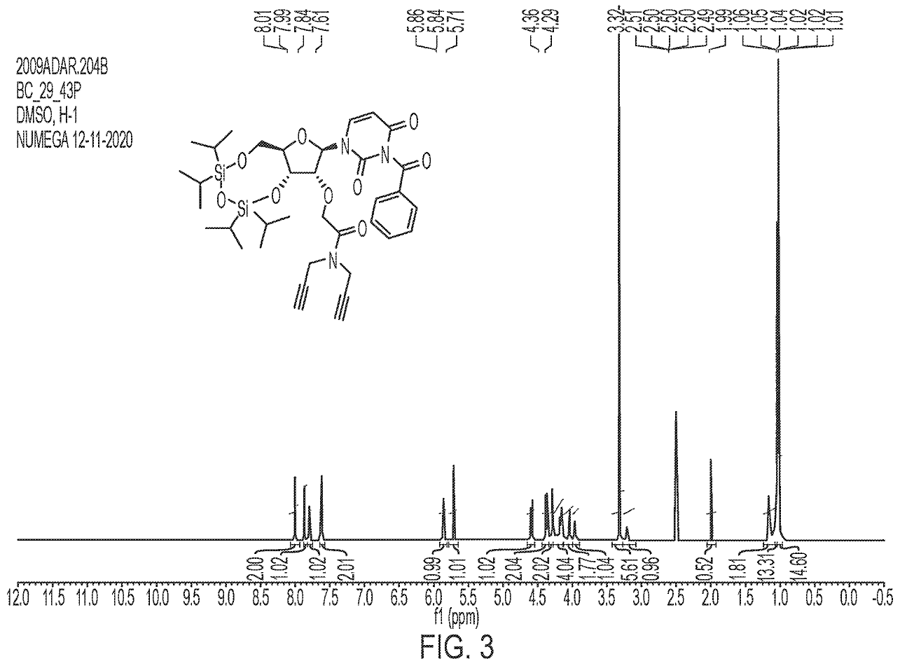

FIG. 4 shows a ¹H NMR spectrum of product 6 from Example 3.

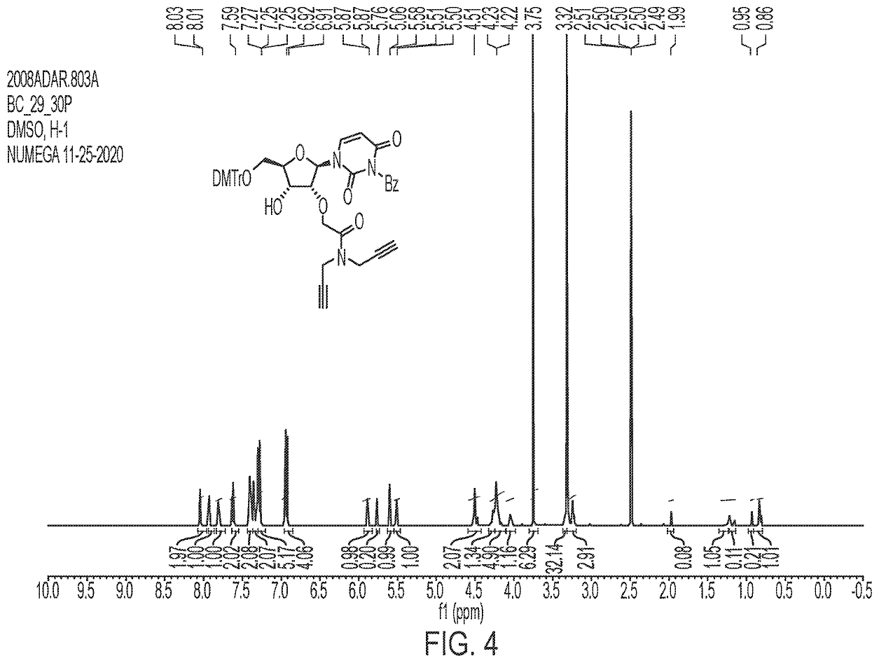

FIG. 5 shows a ¹H NMR spectrum of product 7 from Example 3.

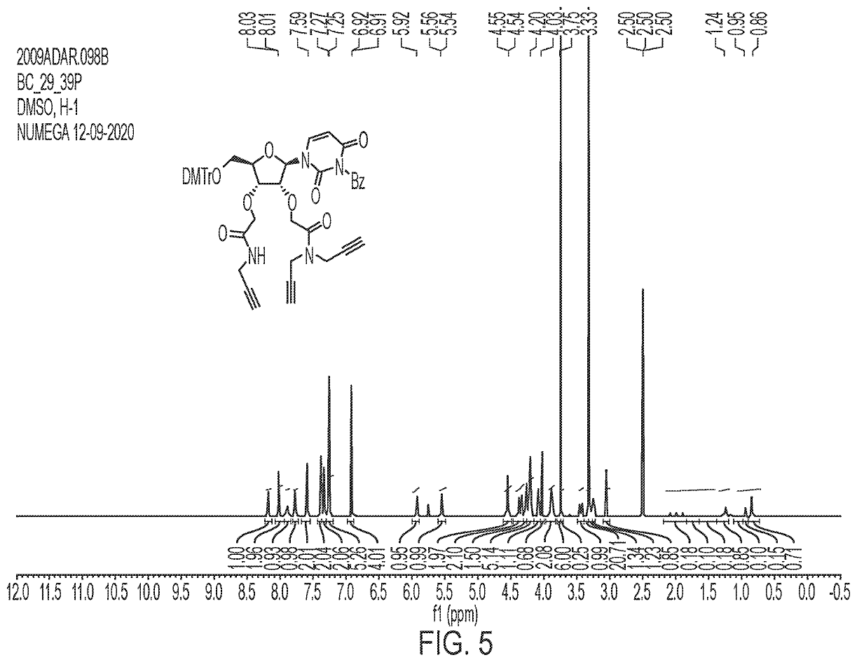

FIG. 6 shows a ¹H NMR spectrum of product 8 from Example 3.

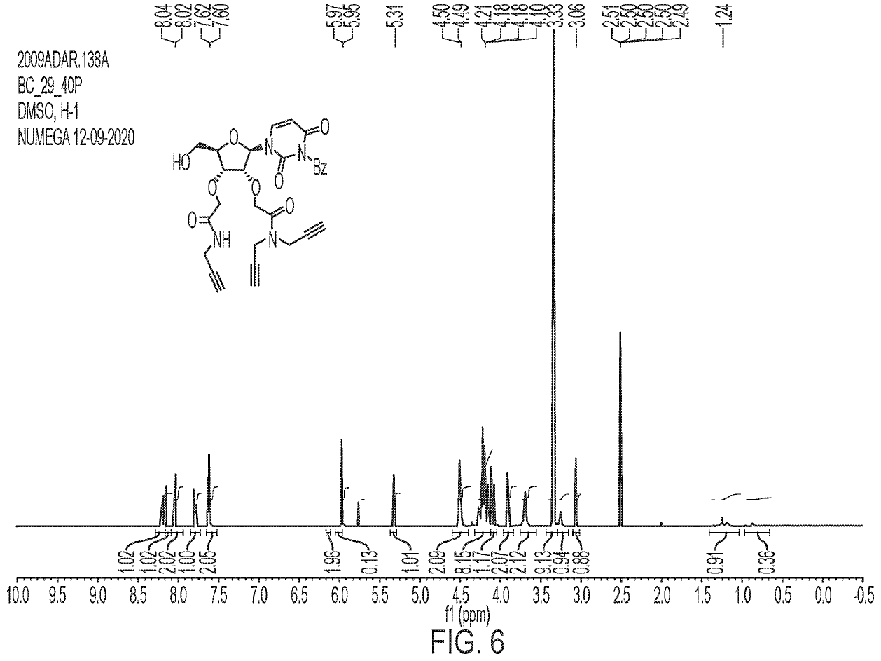

FIG. 7 shows a ¹H NMR spectrum of product 10 from Example 3.

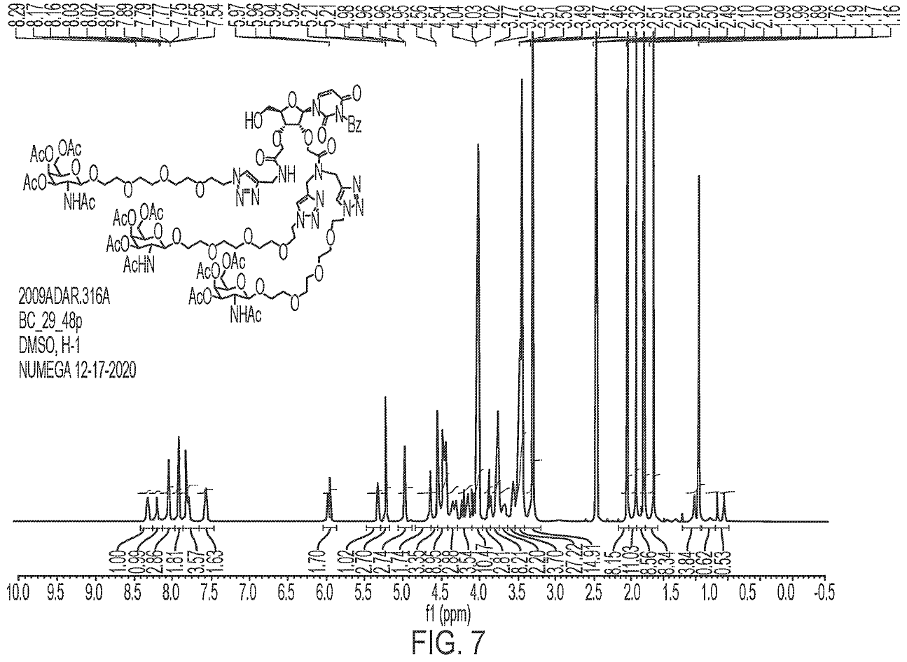

FIG. 8 shows tandem mass spectrometry of product 10 from Example 3.

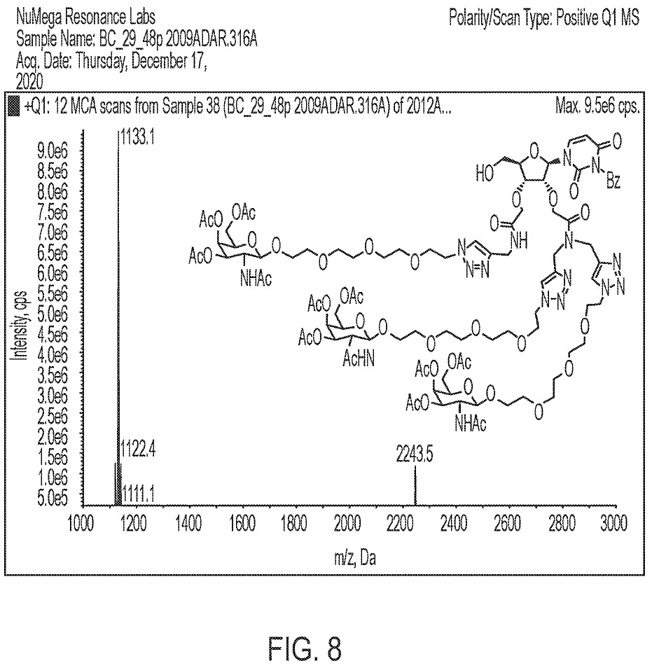

FIG. 9 shows tandem mass spectrometry of product 11 from Example 3.

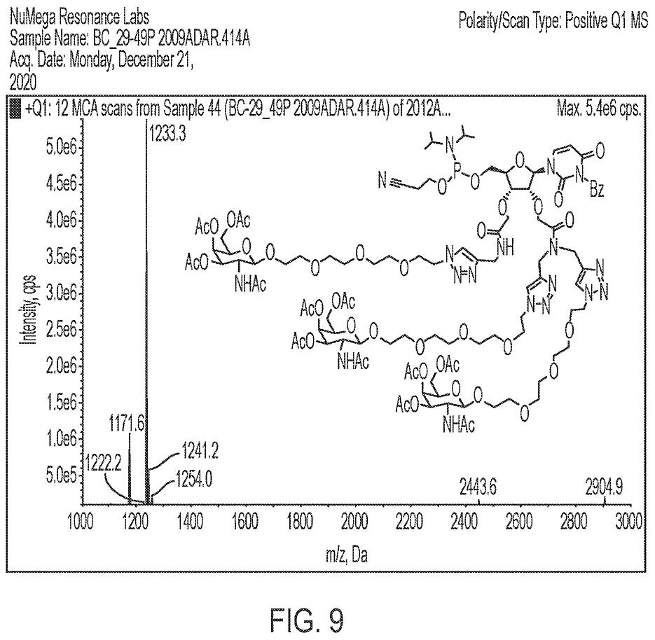

FIG. 10 shows a ¹H NMR spectrum of product 11 from Example 3.

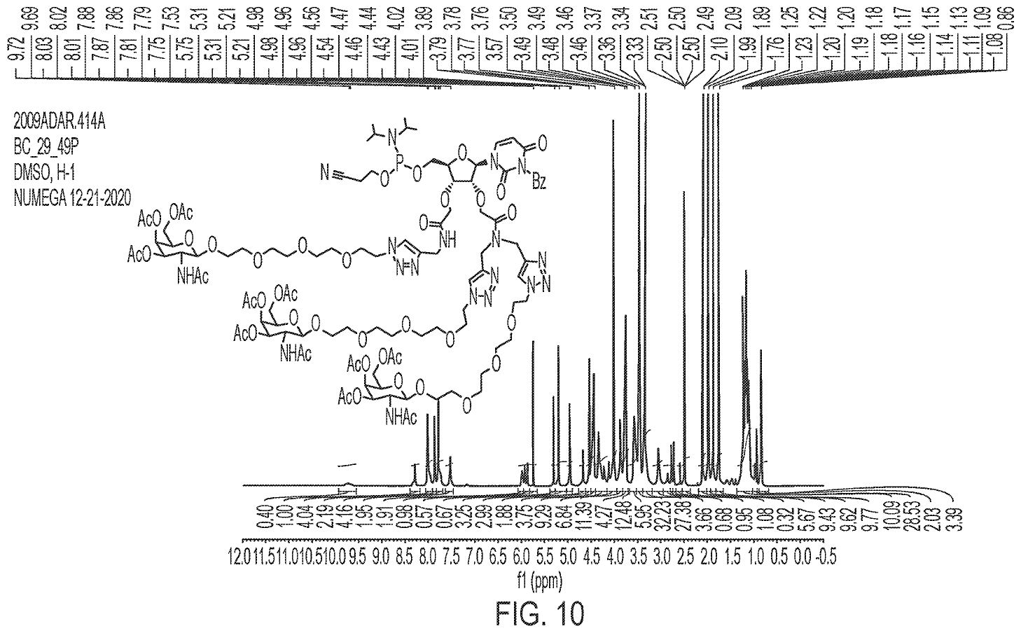

FIG. 11 shows a ¹³C NMR spectrum of product 11 from Example 3.

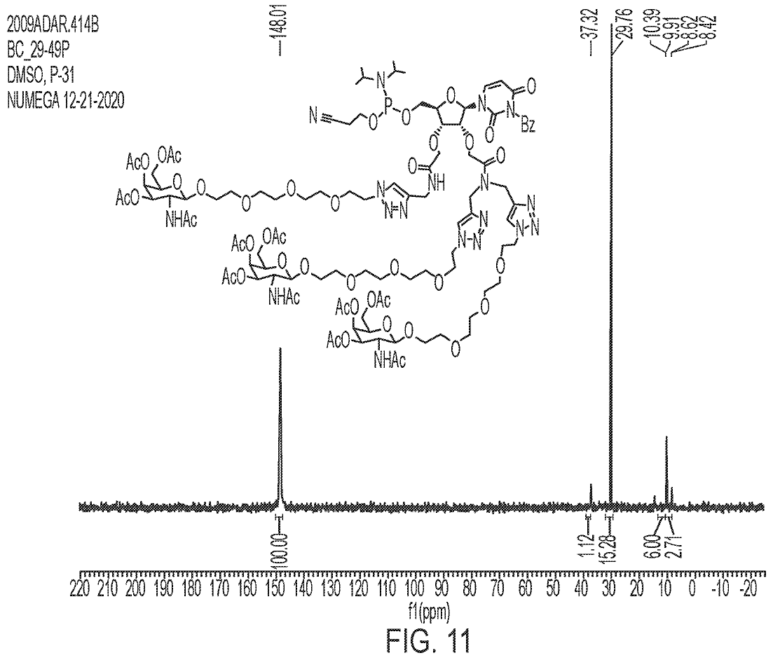

FIG. 12 shows a ¹H NMR spectrum of product 2 from Example 4.

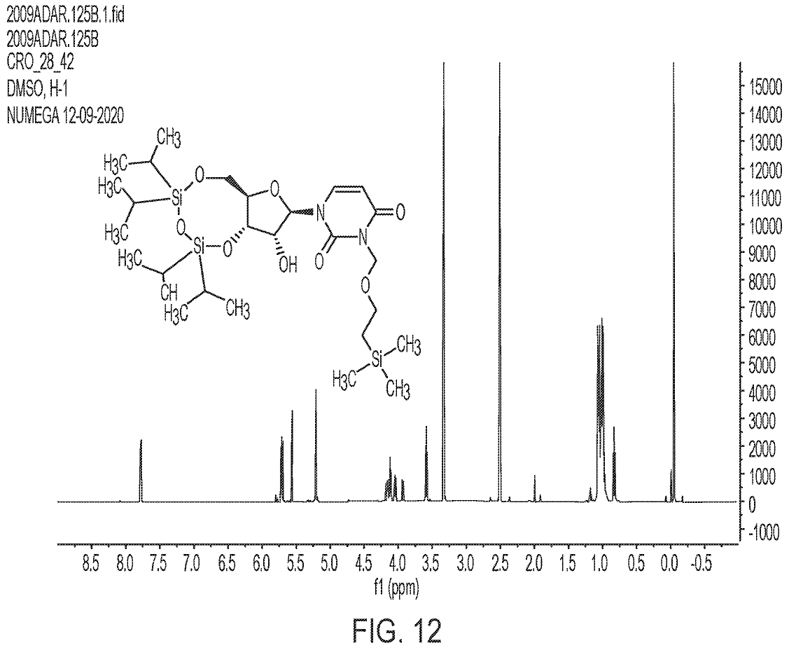

FIG. 13 shows a ¹H NMR spectrum of product 3 from Example 4.

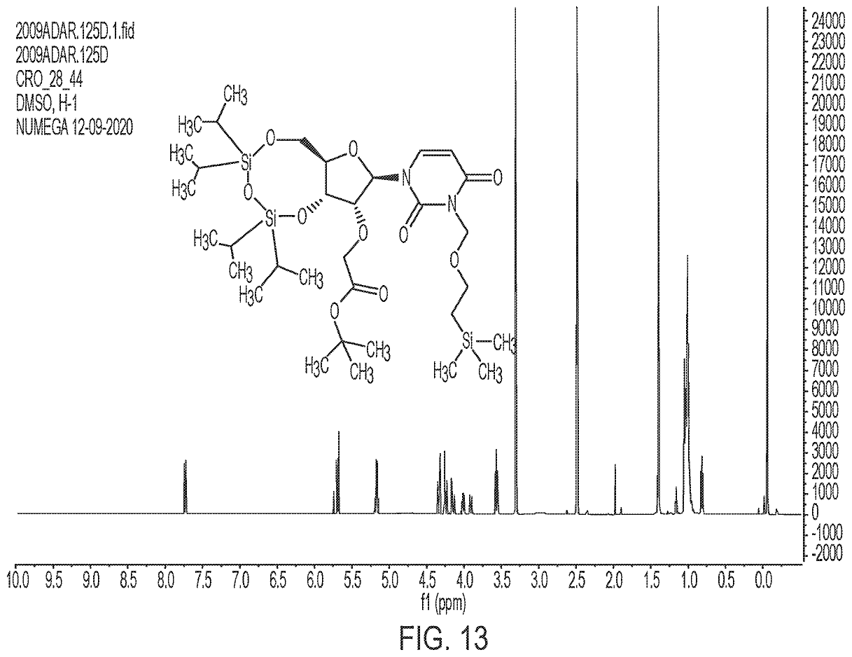

FIG. 14 shows a ¹H NMR spectrum of product 4 from Example 4.

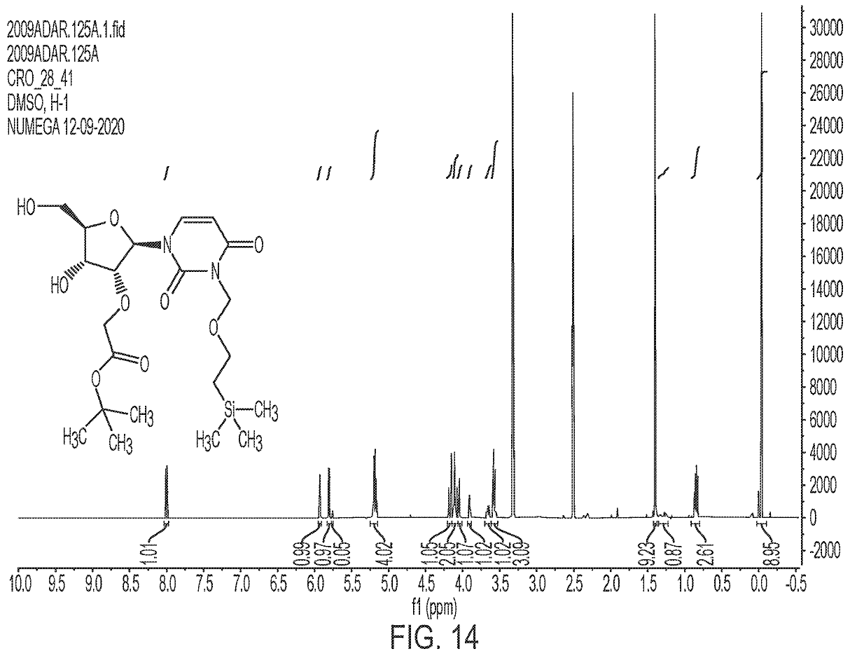

FIG. 15 shows a ¹H NMR spectrum of product 5 from Example 4.

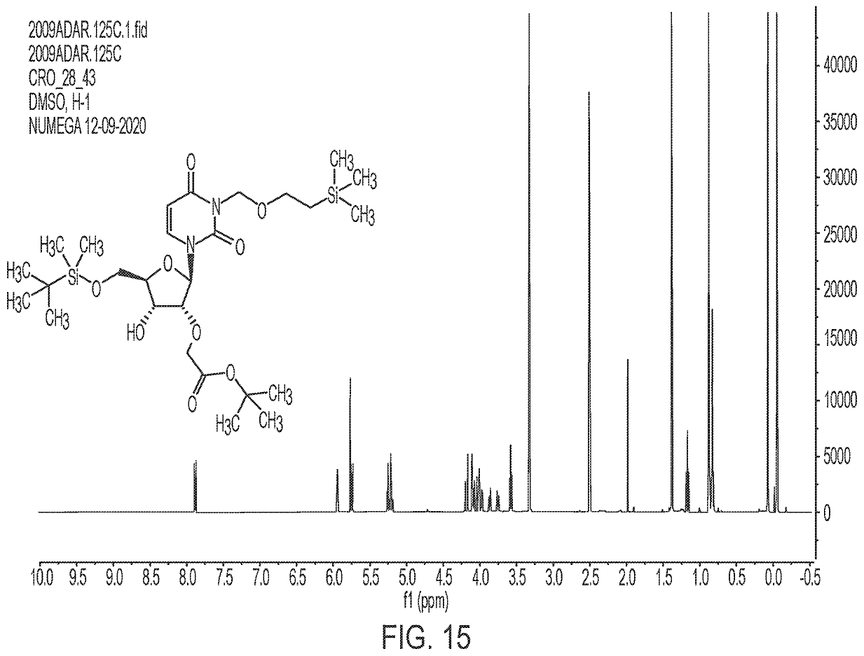

FIG. 16 shows a ¹H NMR spectrum of product 6 from Example 4.

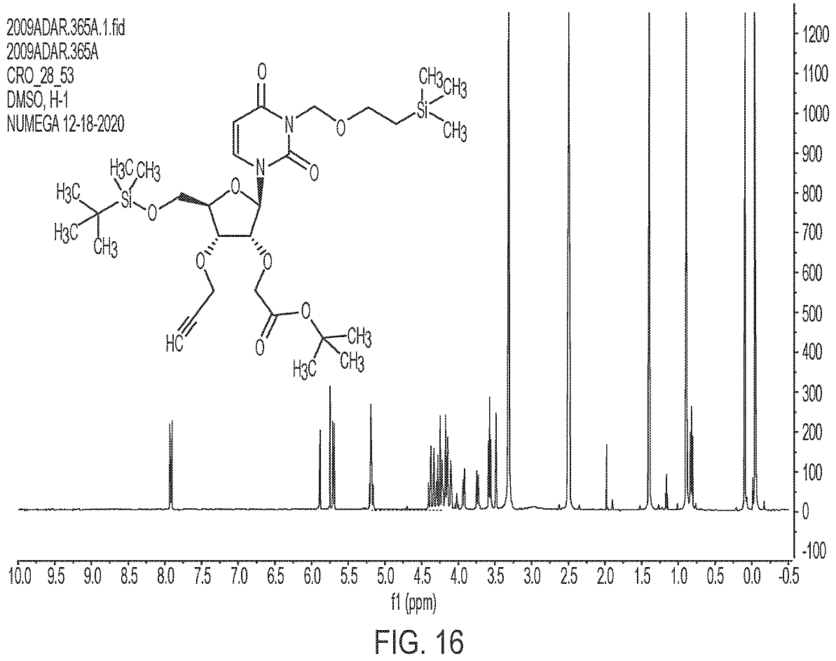

FIG. 17 shows a ¹H NMR spectrum of product 7 from Example 4.

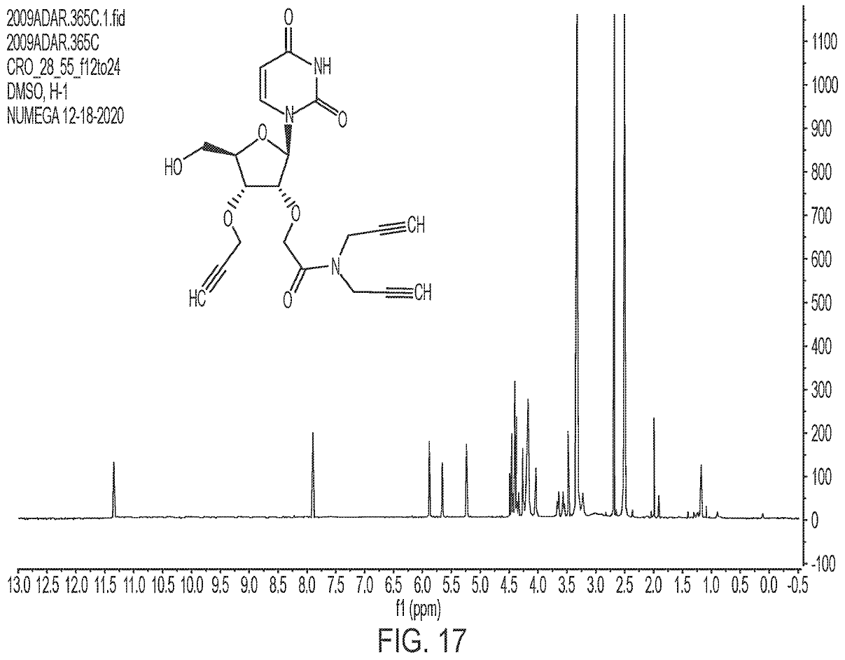

FIG. 18 shows a ¹H NMR spectrum of product 8 from Example 4.

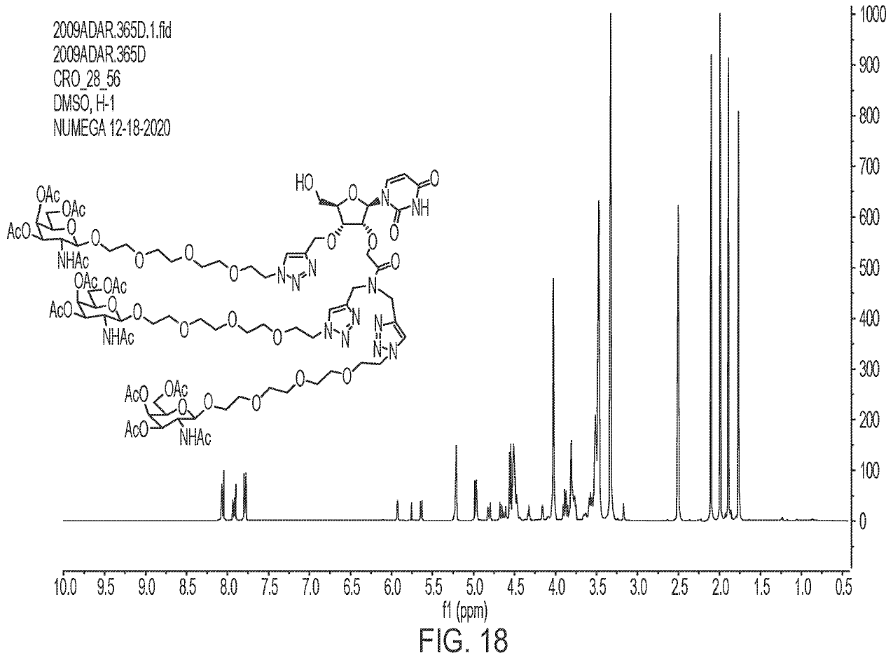

FIG. 19 shows a ¹H NMR spectrum of product 10 from Example 4.

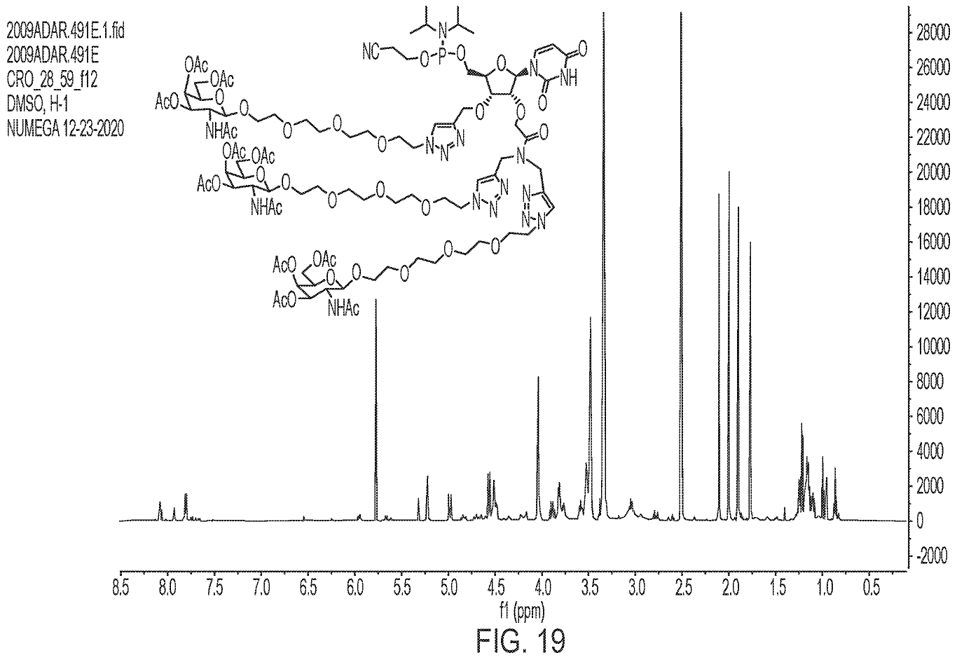

FIG. 20 shows tandem mass spectrometry of product 10 from Example 4.

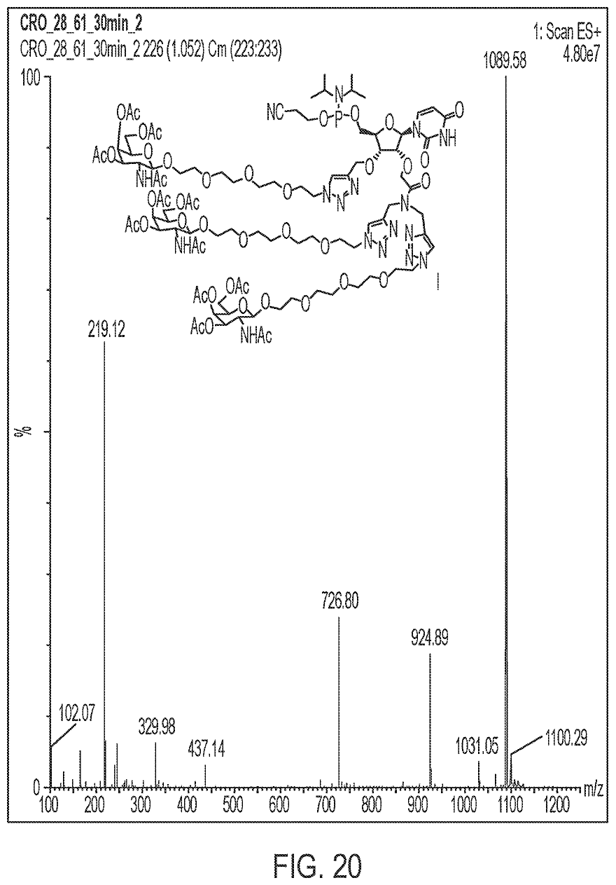

FIG. 21 shows a ¹H NMR spectrum of compound 6 from Example 7.

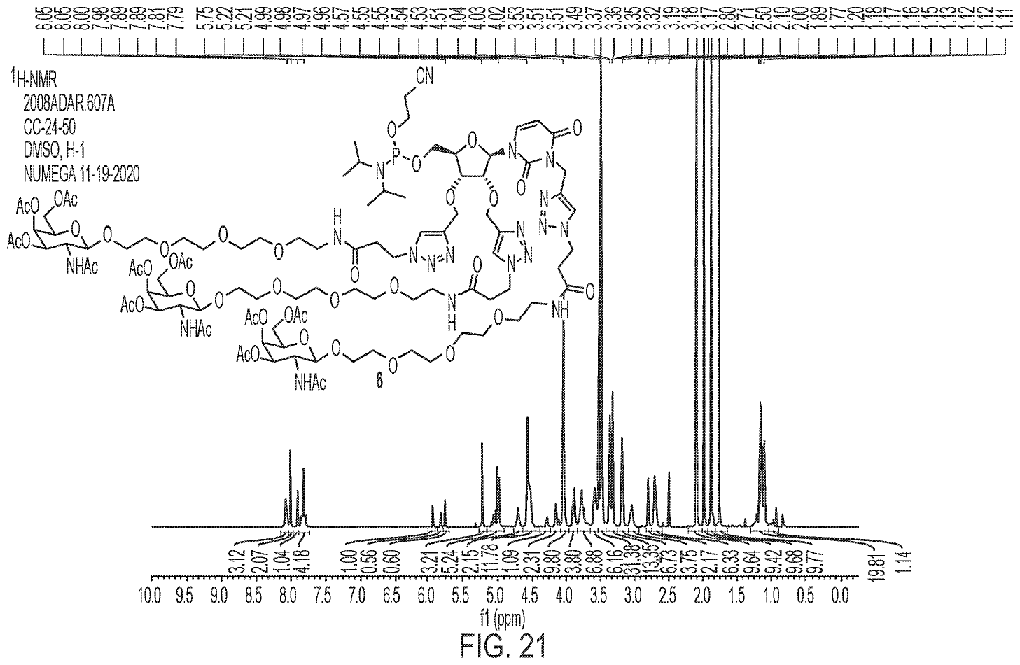

FIG. 22 shows a ³¹P NMR spectrum of compound 5 from Example 7.

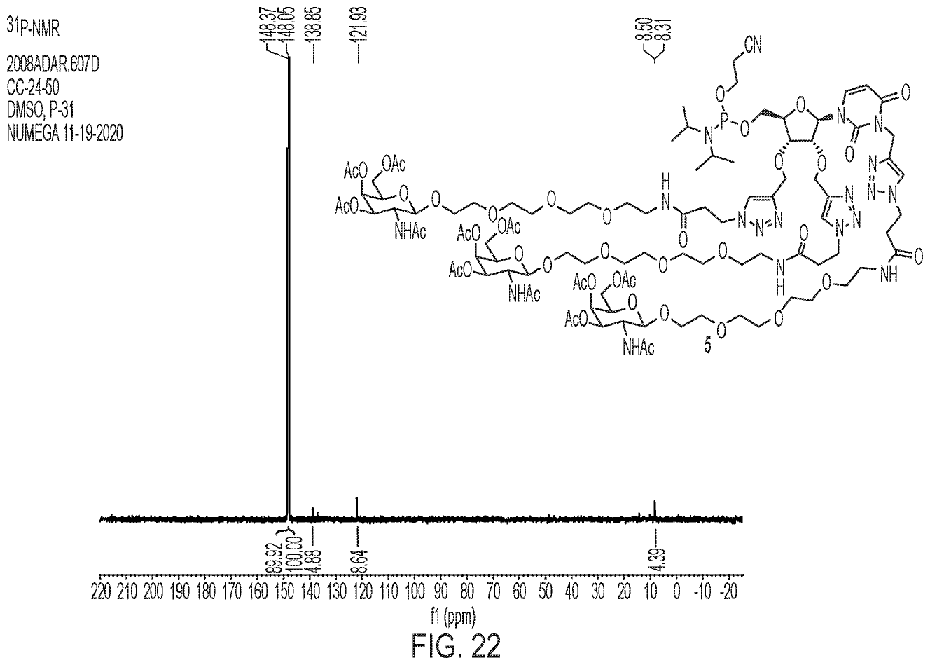

FIG. 23 shows a ¹H NMR spectrum of compound 2 from Example 7.

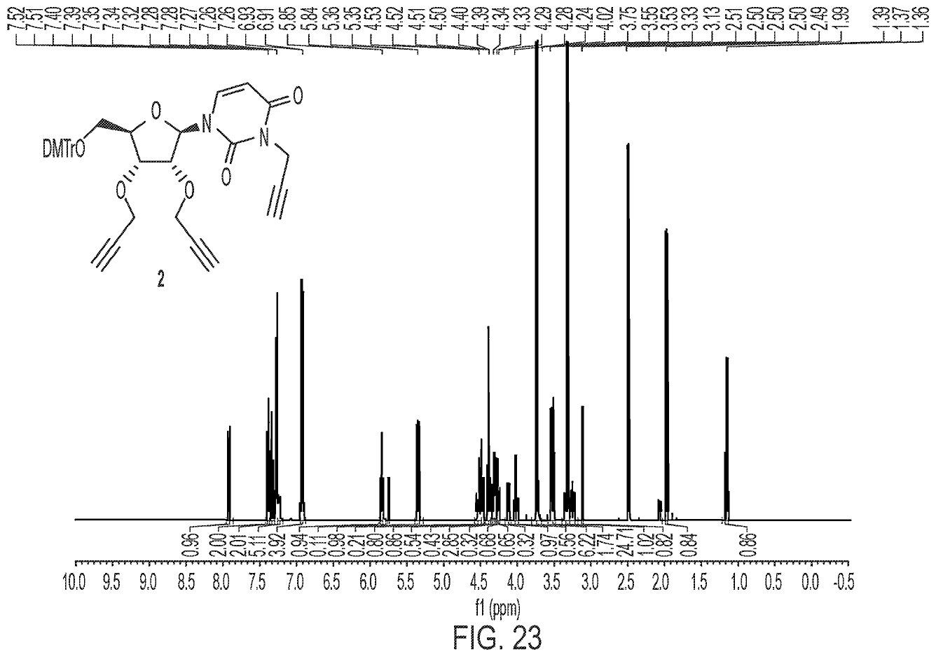

FIG. 24 shows a ¹H NMR spectrum of compound 3 from Example 7.

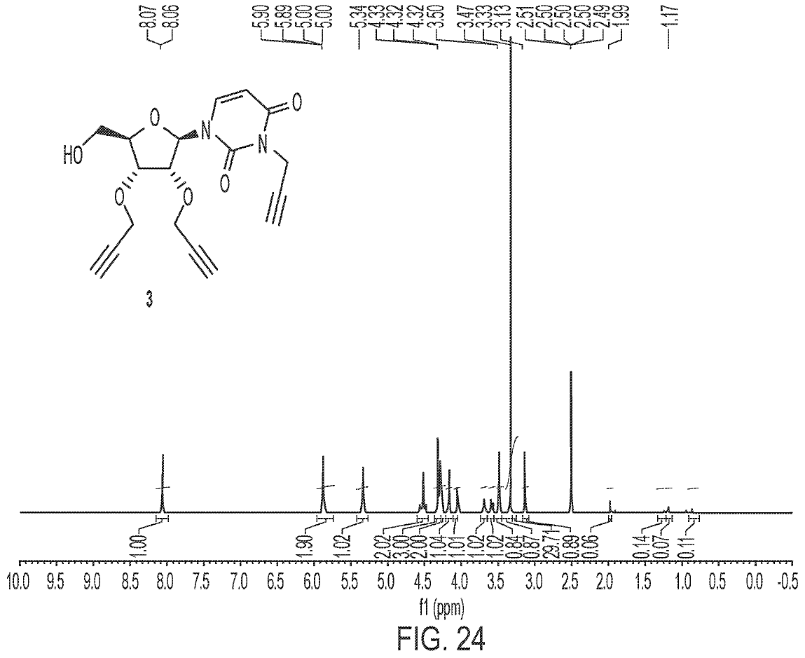

FIG. 25 shows a ¹H NMR spectrum of GalNAc-[PEG]₄-NH₂ trifluoroacetate from Example 7.

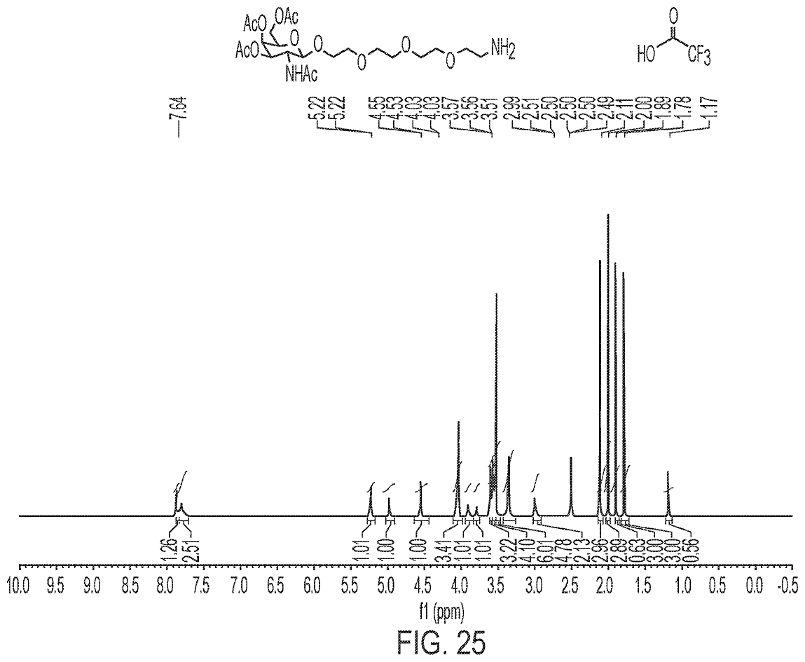

FIG. 26 shows a ¹H NMR spectrum of compound 10 from Example 7.

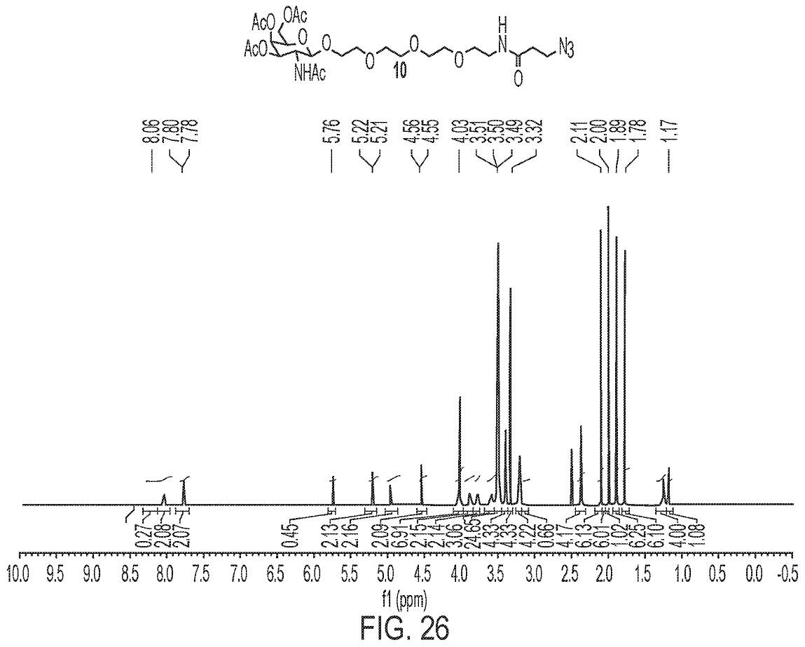

FIG. 27 shows a ¹H NMR spectrum of compound 4 from Example 7.

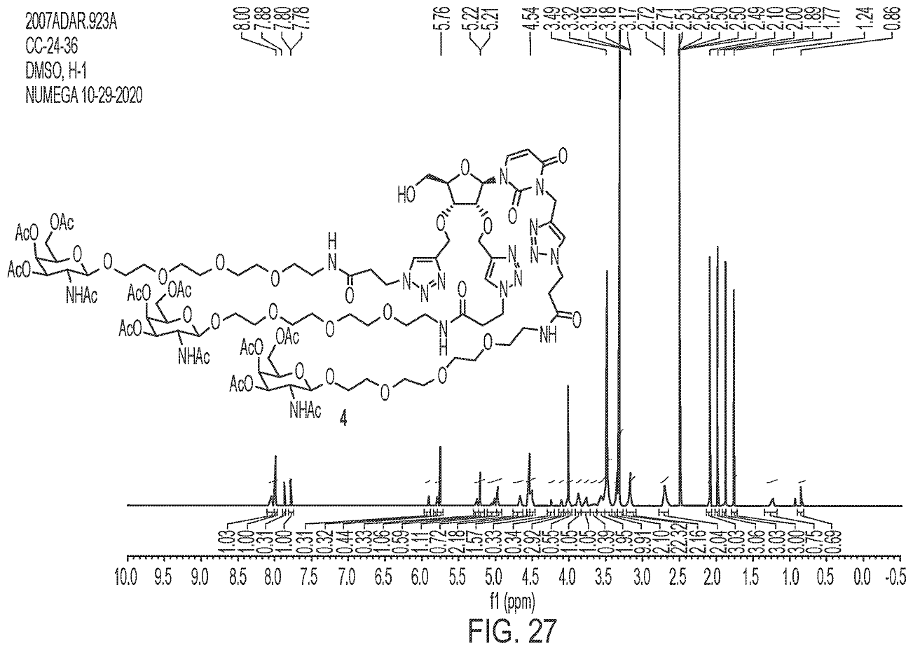

FIG. 28 shows a ¹H NMR spectrum of compound 2 from Example 8.

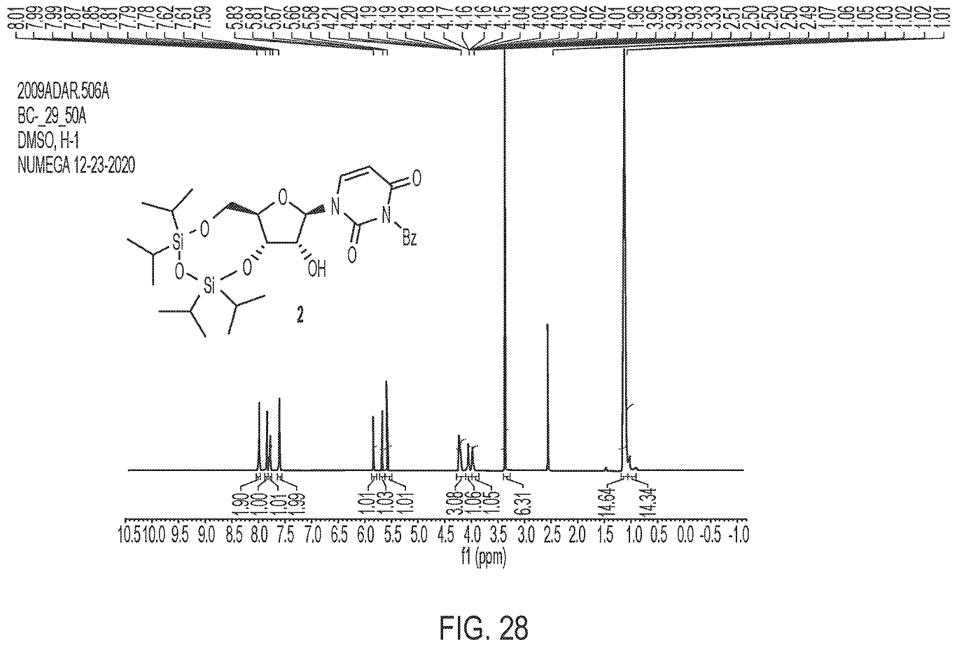

FIG. 29 shows a ¹H NMR spectrum of compound 3 from Example 8.

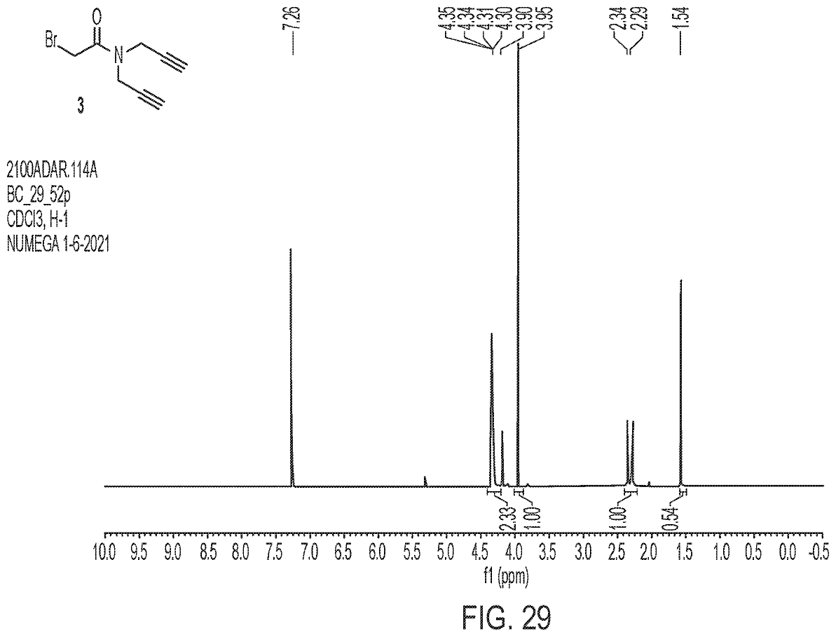

FIG. 30 shows a ¹H NMR spectrum of compound 4 from Example 8.

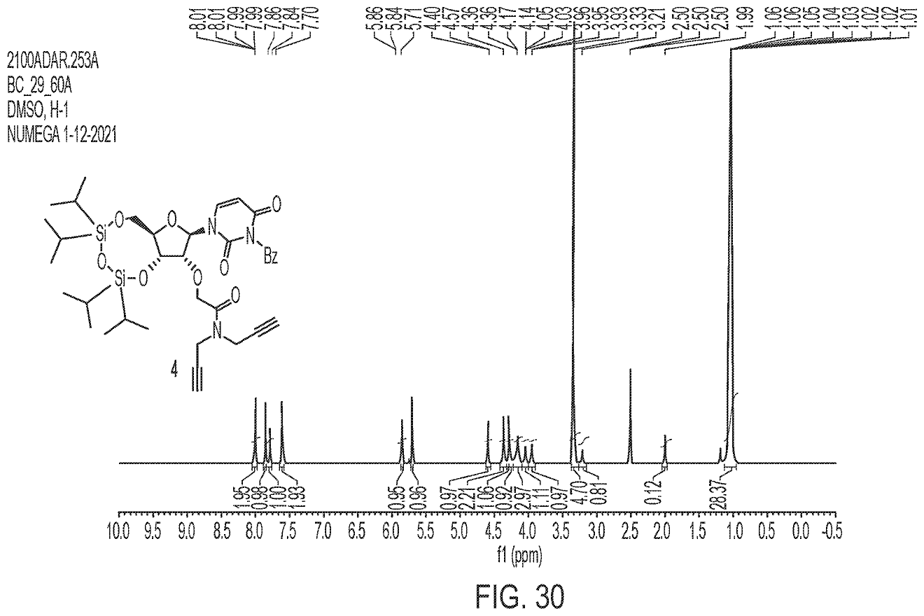

FIG. 31 shows a ¹H NMR spectrum of compound 5 from Example 8.

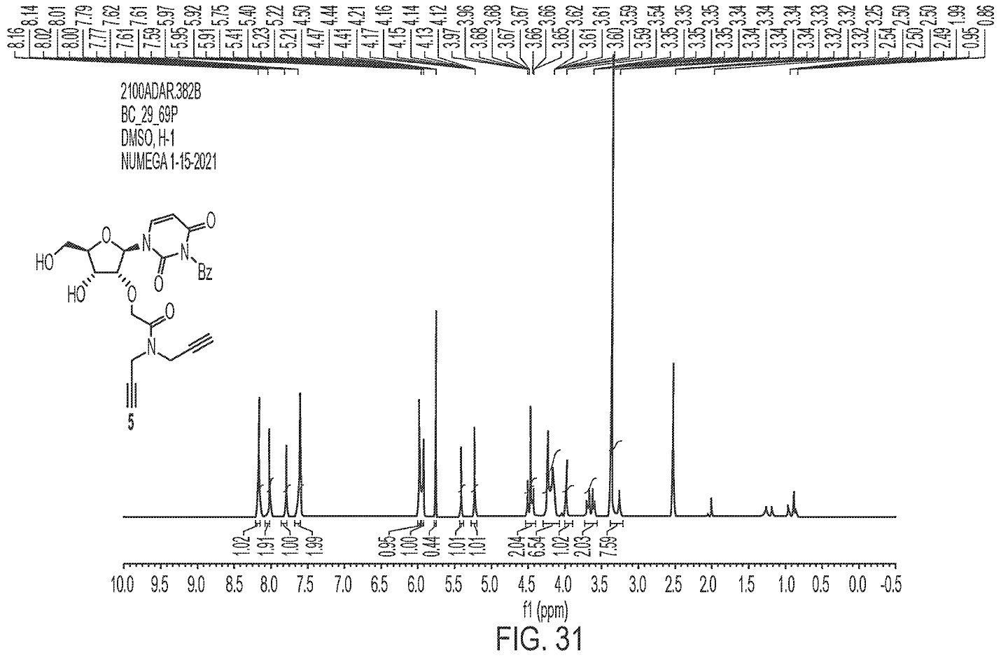

FIG. 32 shows a ¹H NMR spectrum of compound 6 from Example 8.

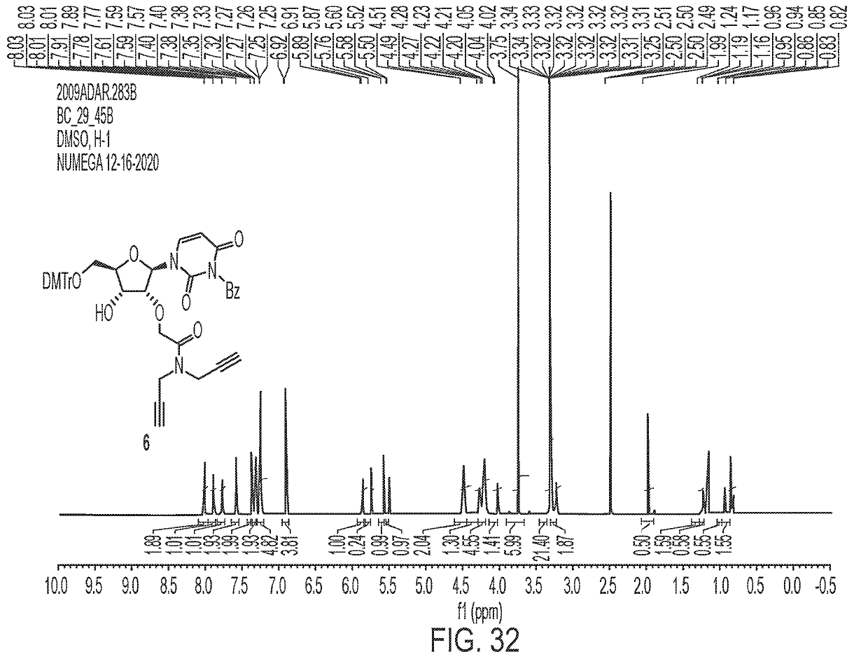

FIG. 33 shows a ¹H NMR spectrum of compound 8 from Example 8.

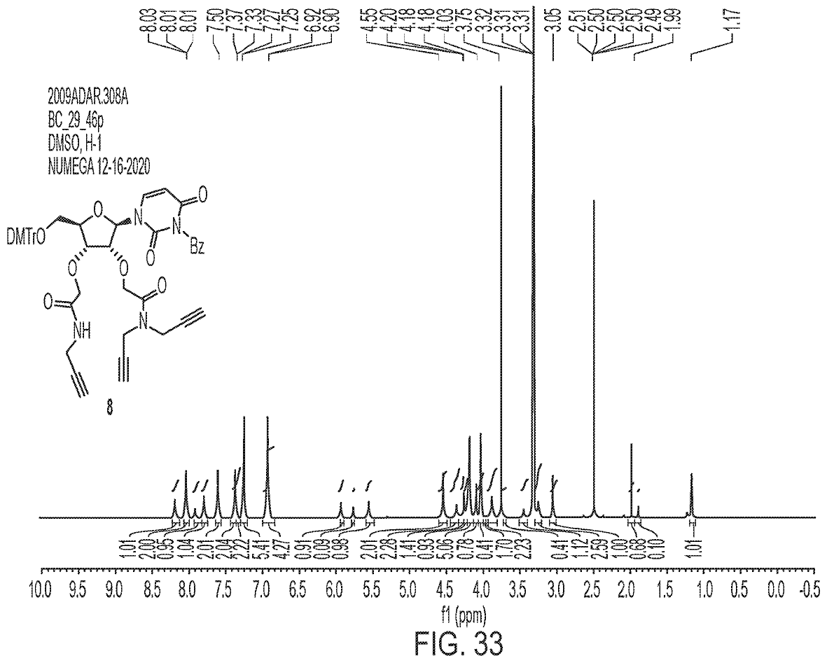

FIG. 34 shows a ¹H NMR spectrum of compound 9 from Example 8.

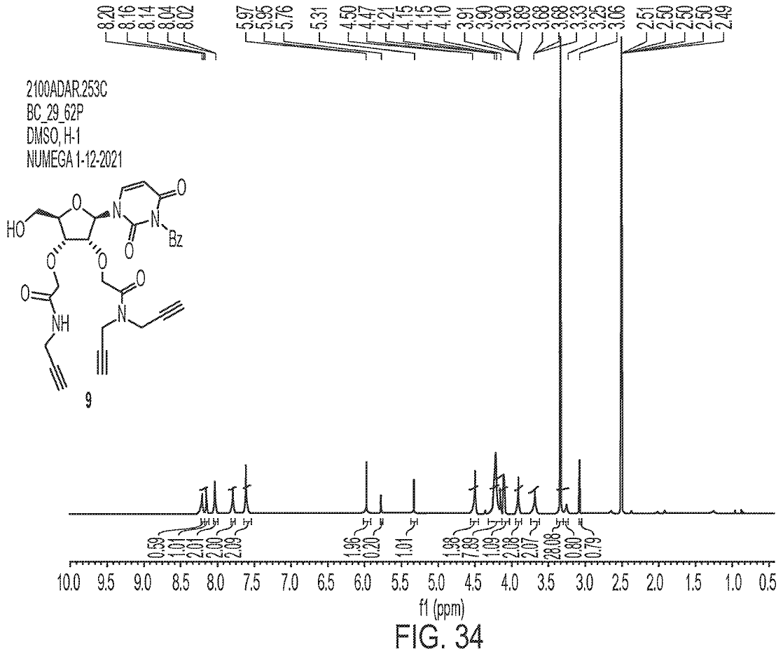

FIG. 35 shows a ¹H NMR spectrum of compound 12 from Example 8.

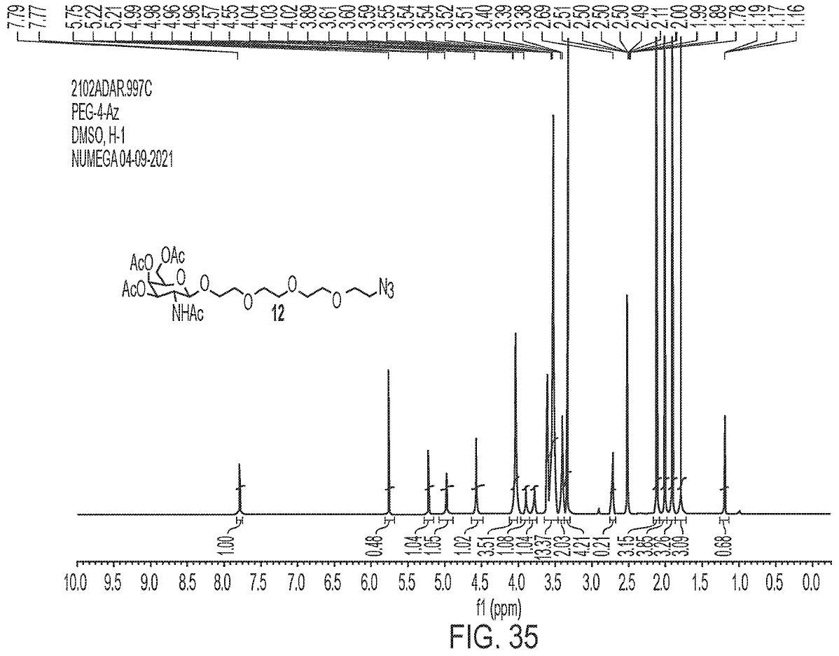

FIG. 36 shows a ¹H NMR spectrum of compound 10 from Example 8.

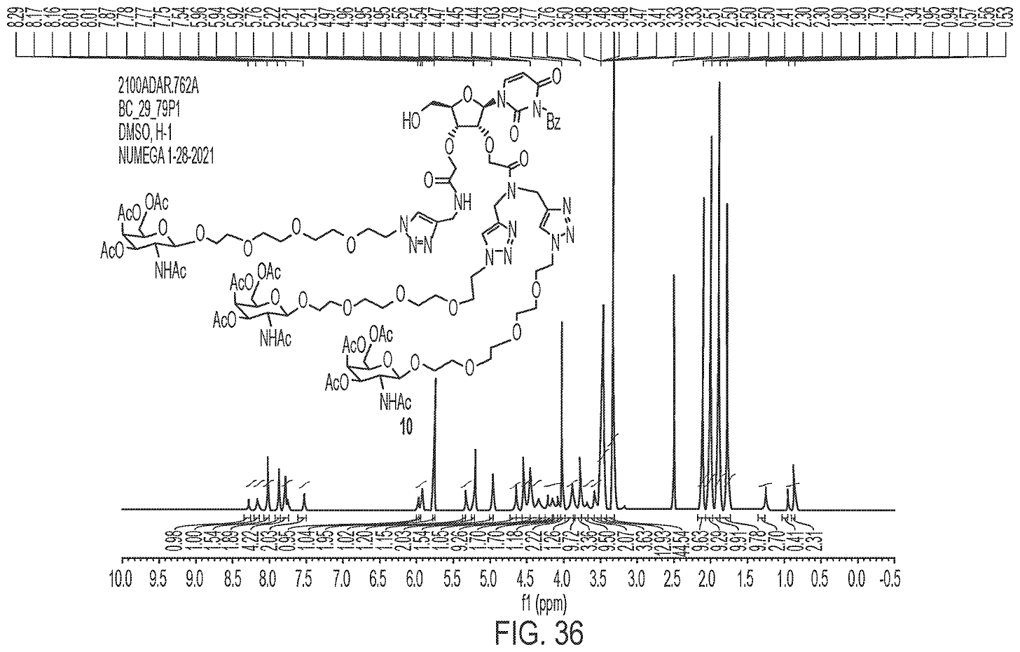

FIG. 37 shows a ¹H NMR spectrum of compound HA-103 from Example 8.

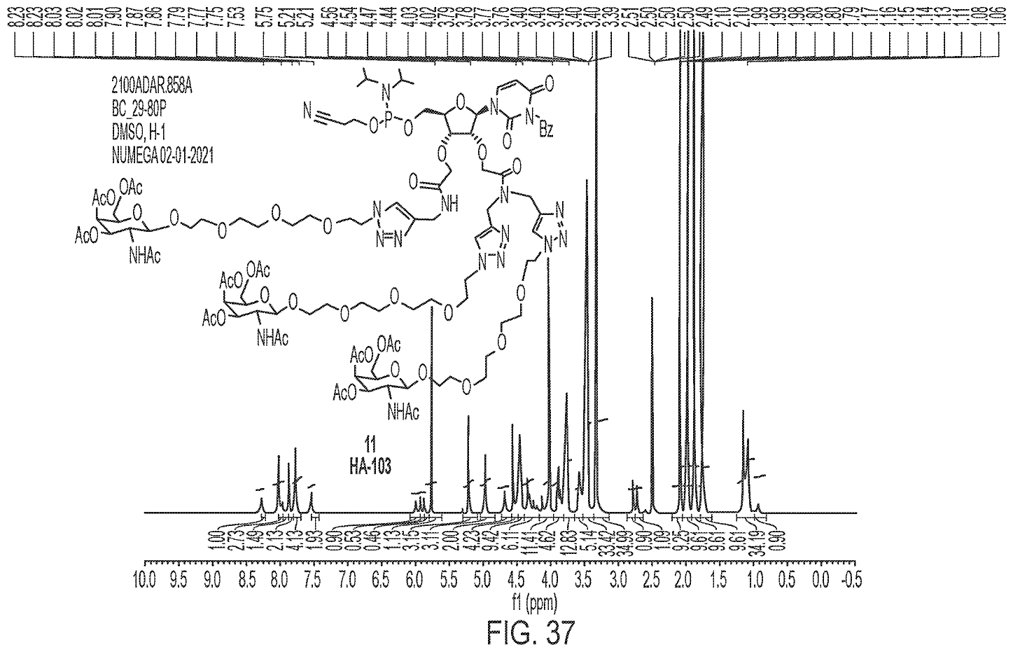

FIG. 38 shows a ³¹P NMR spectrum of compound HA-103 from Example 8.

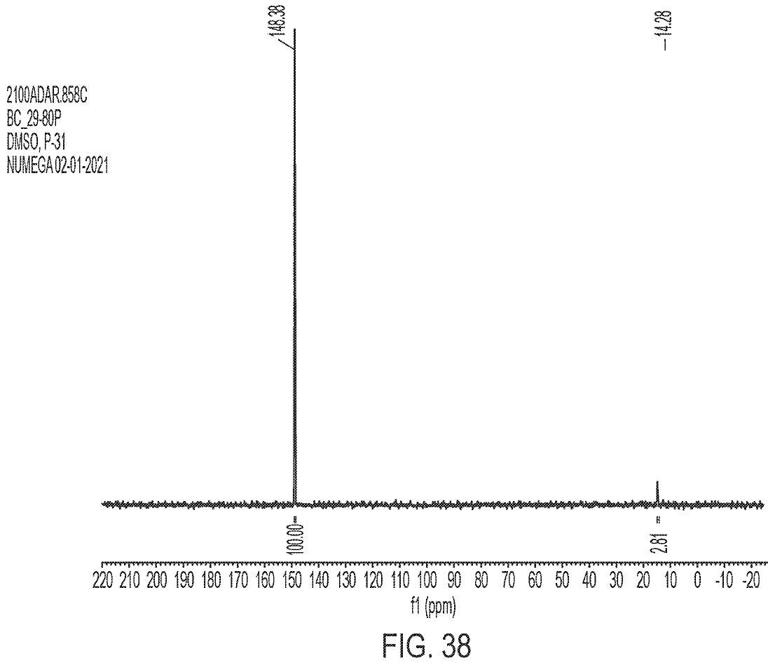

FIG. 39 shows a ¹H NMR spectrum of compound 2 from Example 9.

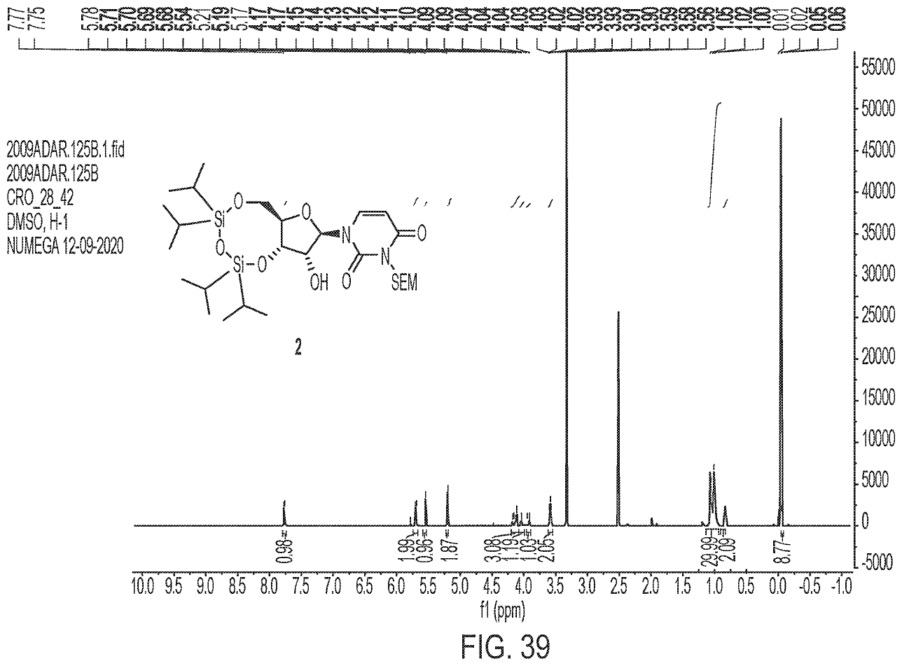

FIG. 40 shows a ¹H NMR spectrum of compound 3 from Example 9.

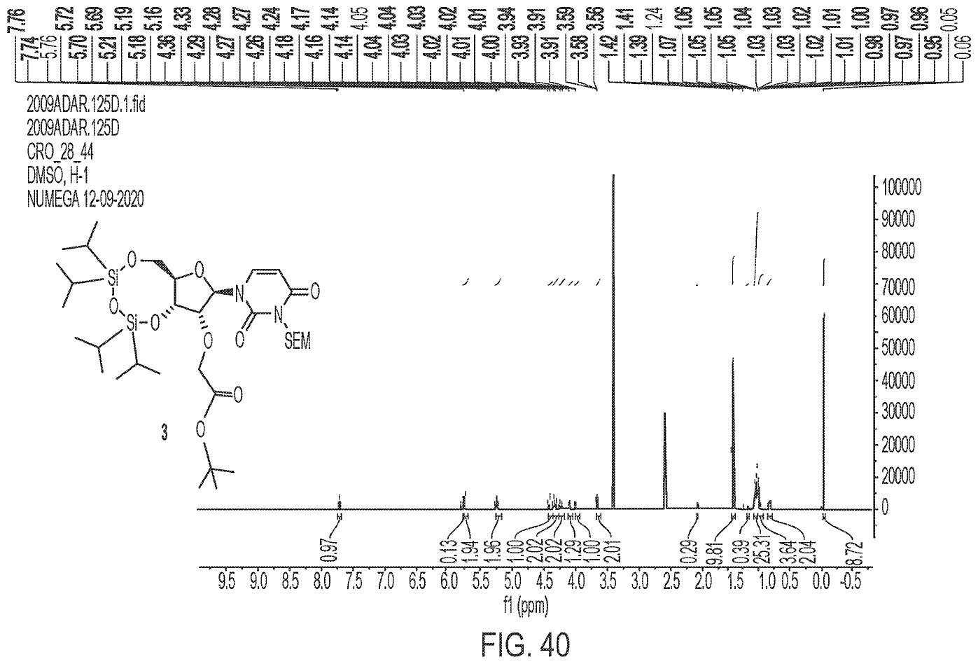

FIG. 41 shows a ¹H NMR spectrum of compound 4 from Example 9.

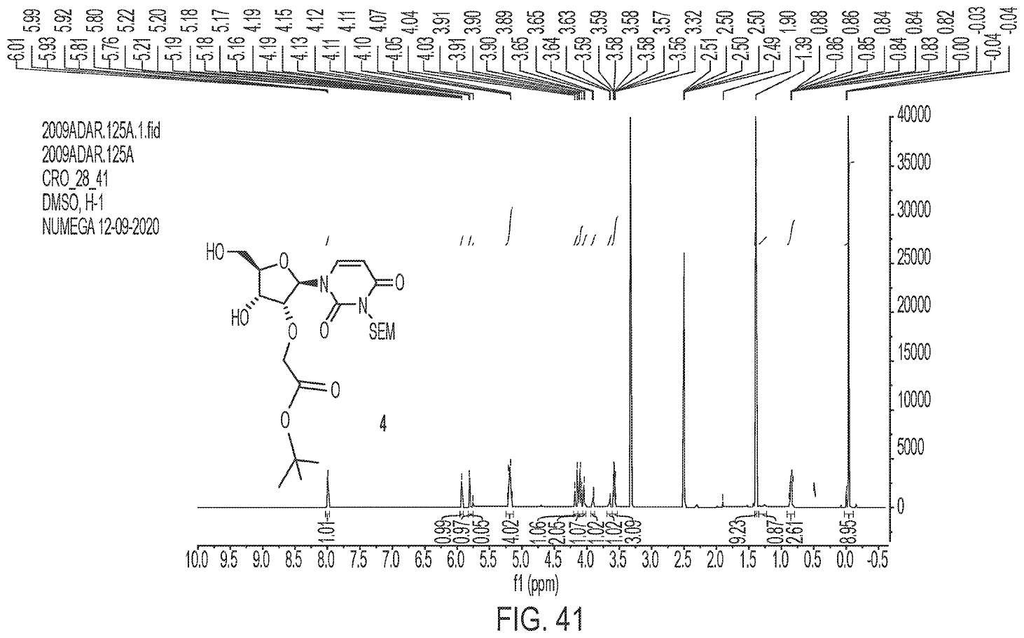

FIG. 42 shows a ¹H NMR spectrum of compound 5 from Example 9.

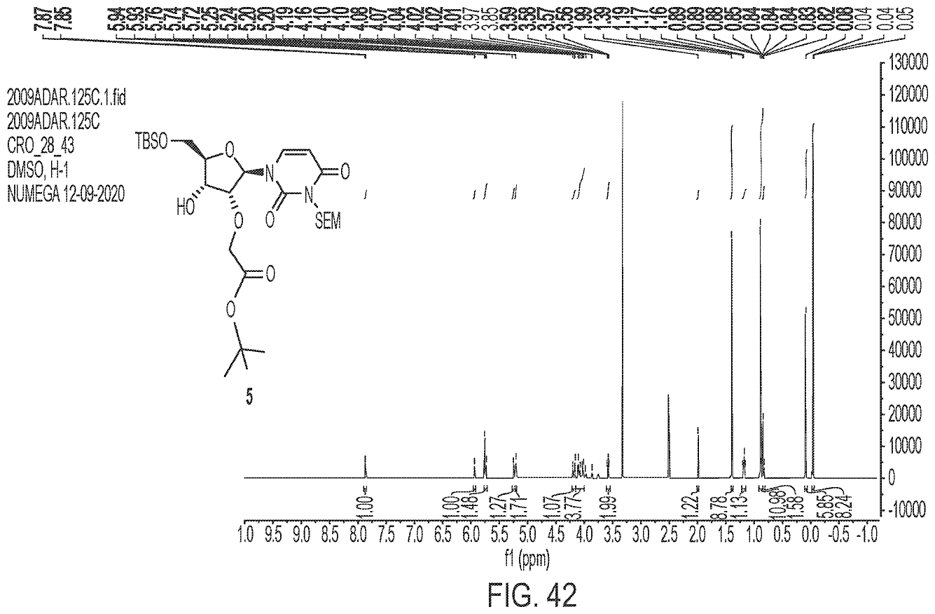

FIG. 43 shows a ¹H NMR spectrum of compound 6 from Example 9.

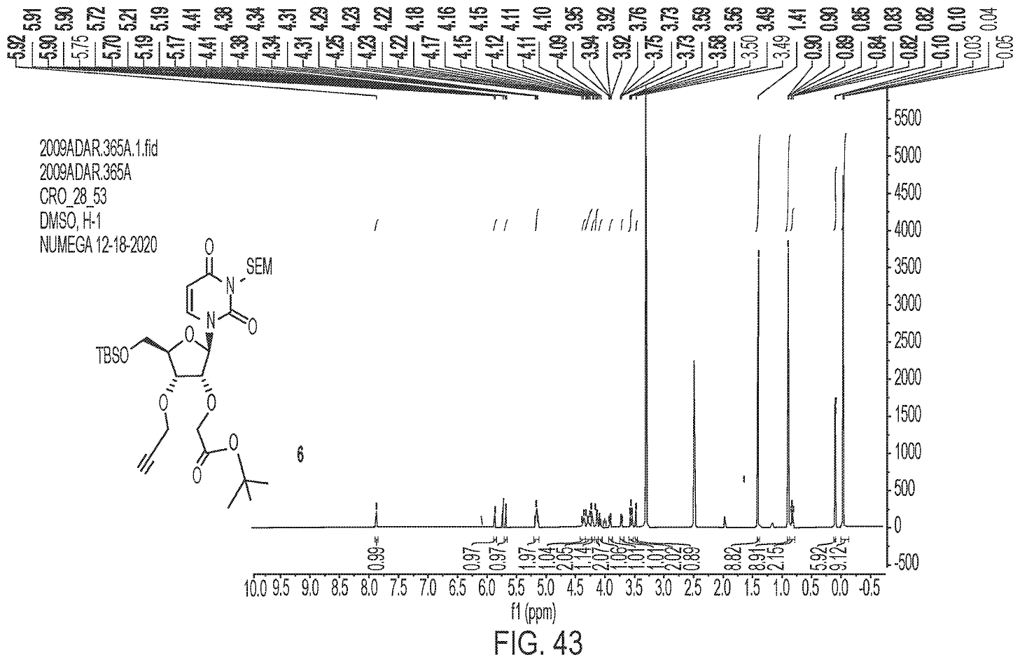

FIG. 44 shows a ¹H NMR spectrum of compound 7 from Example 9.

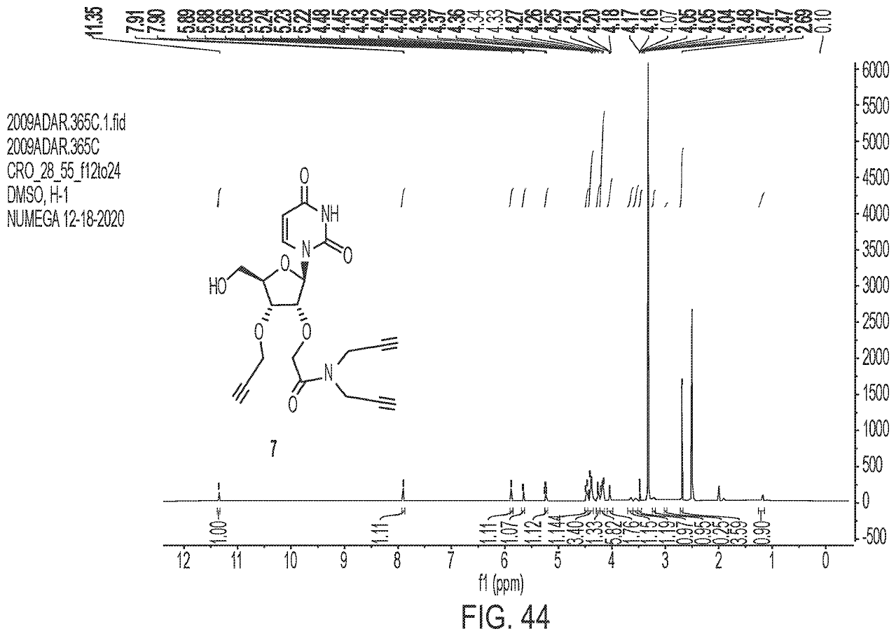

FIG. 45 shows a ¹H NMR spectrum of compound 9 from Example 9.

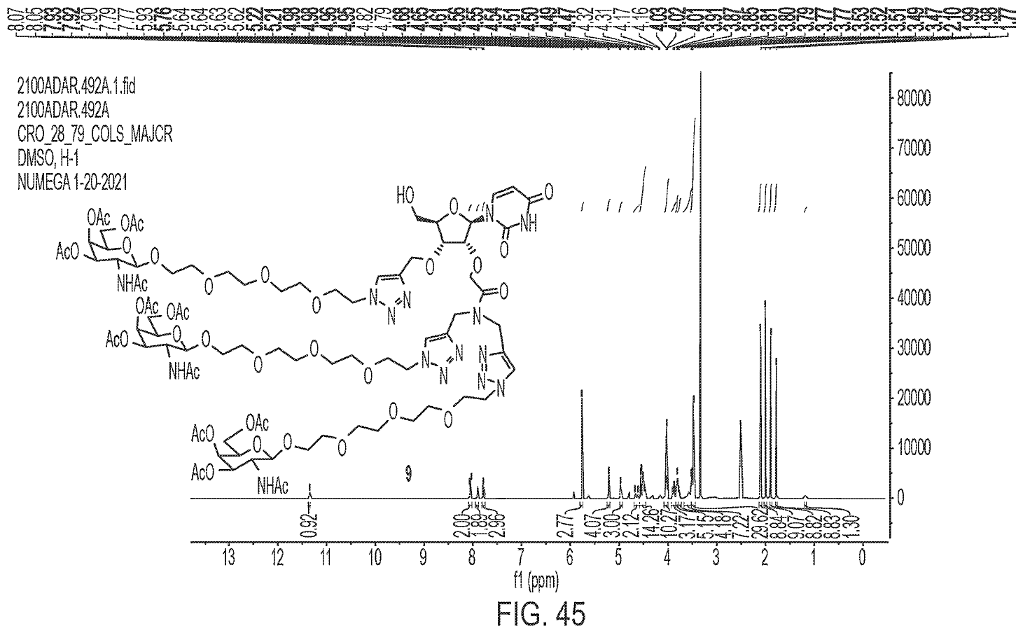

FIG. 46 shows a ¹H NMR spectrum of compound 10 from Example 9.

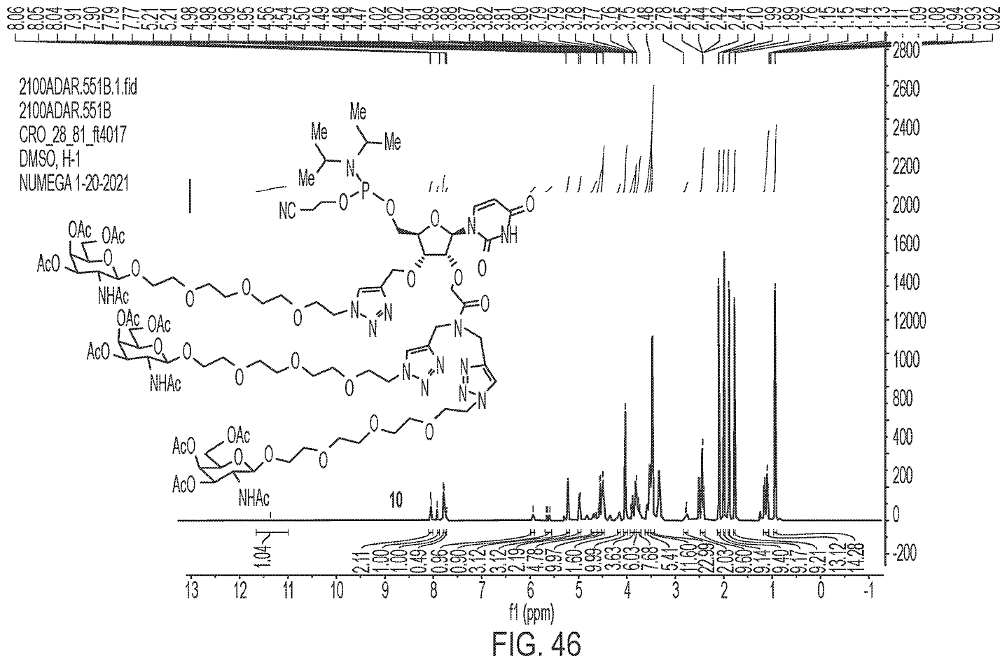

FIG. 47 shows a ³¹P NMR spectrum of compound 10 from Example 9.

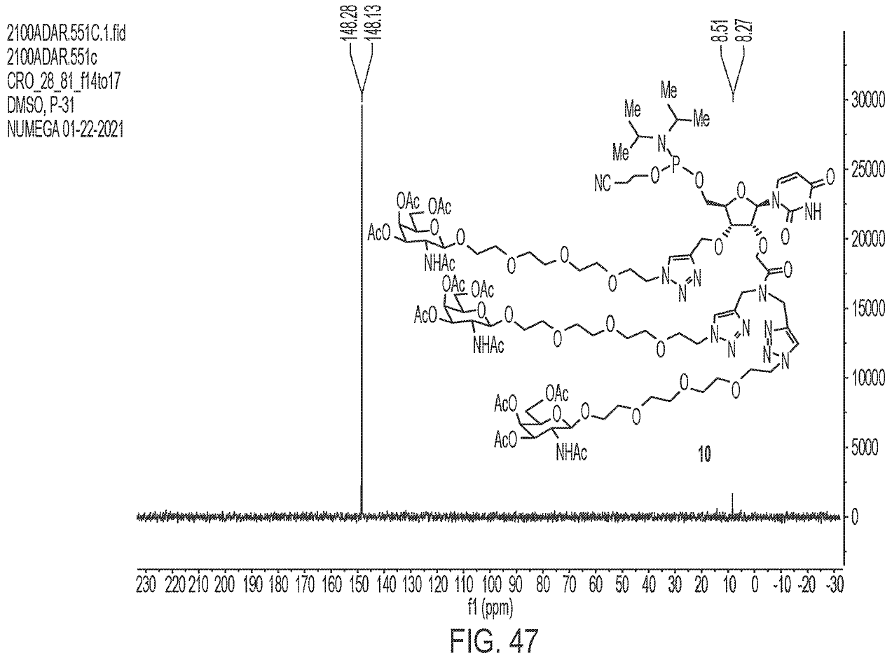

FIG. 48 shows a ¹H NMR spectrum of compound 11 from Example 10.

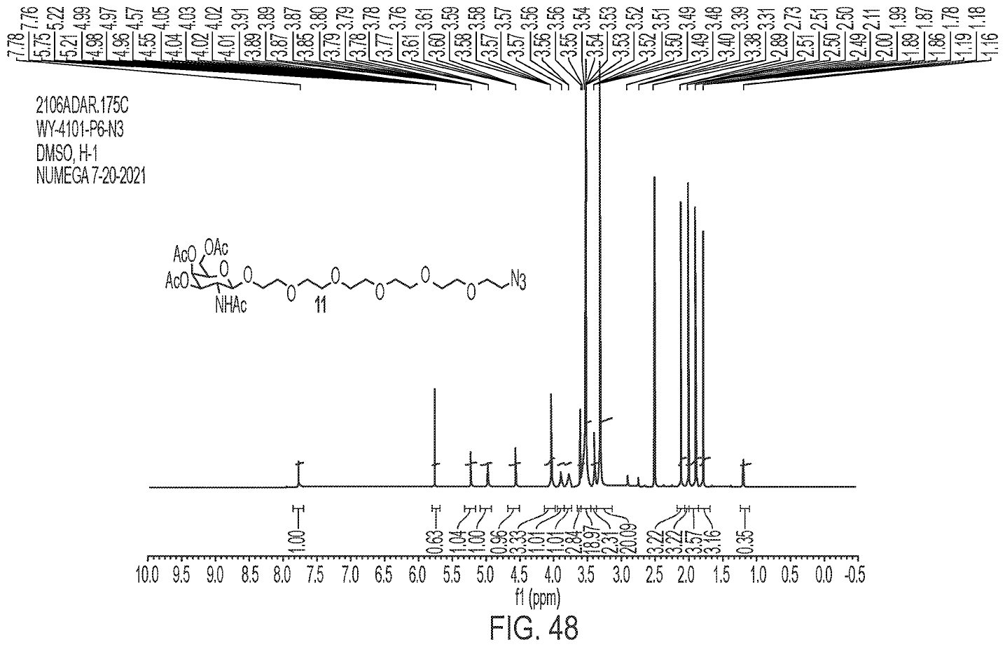

FIG. 49 shows a ¹H NMR spectrum of compound 10 from Example 10.

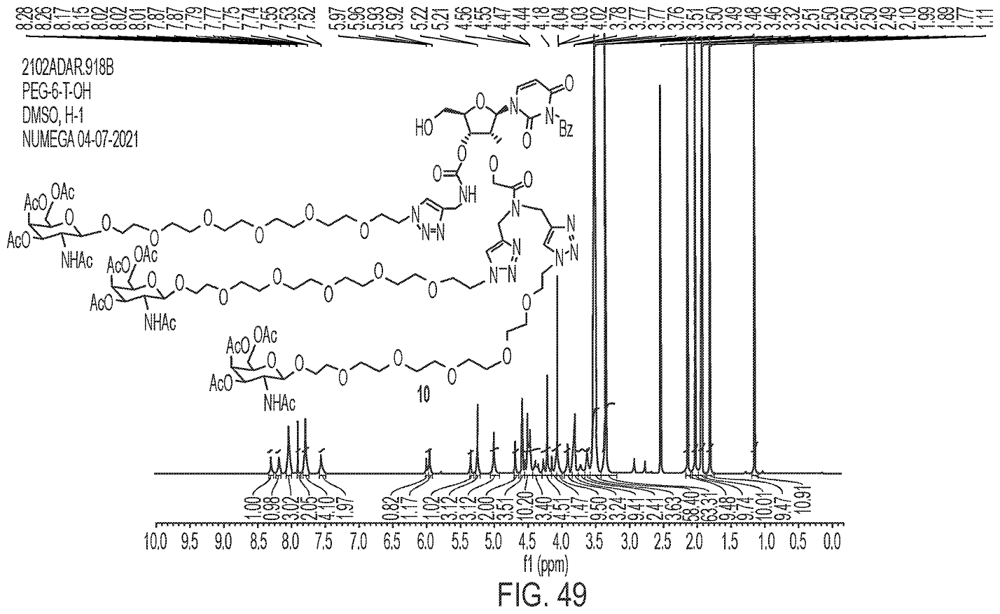

FIG. 50 shows a ¹H NMR spectrum of compound H4-(HA-111) from Example 10.

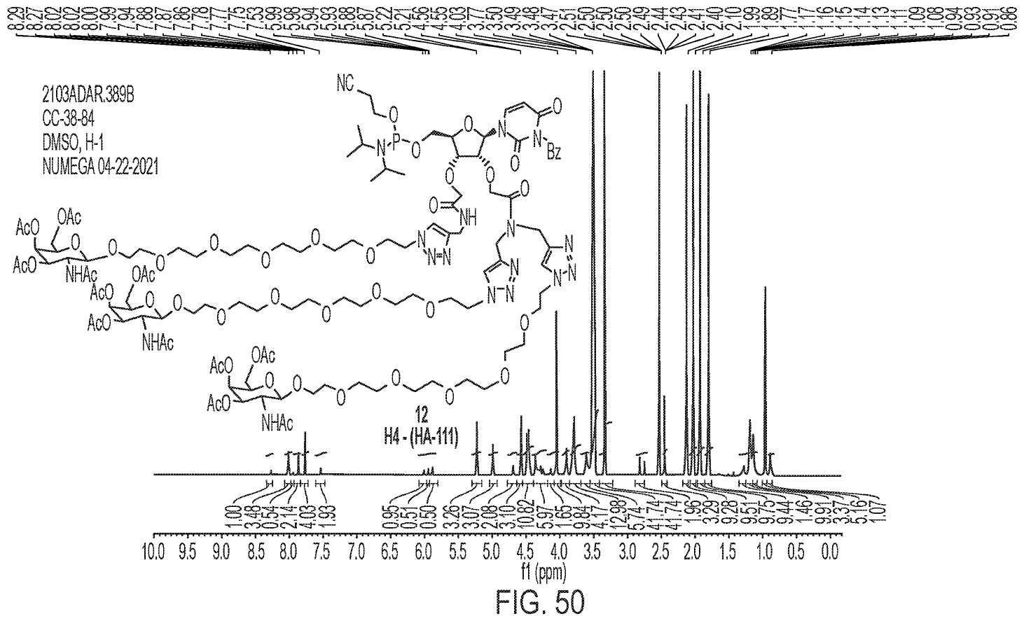

FIG. 51 shows a ³¹P NMR spectrum of compound H4-(HA-111) from Example 10.

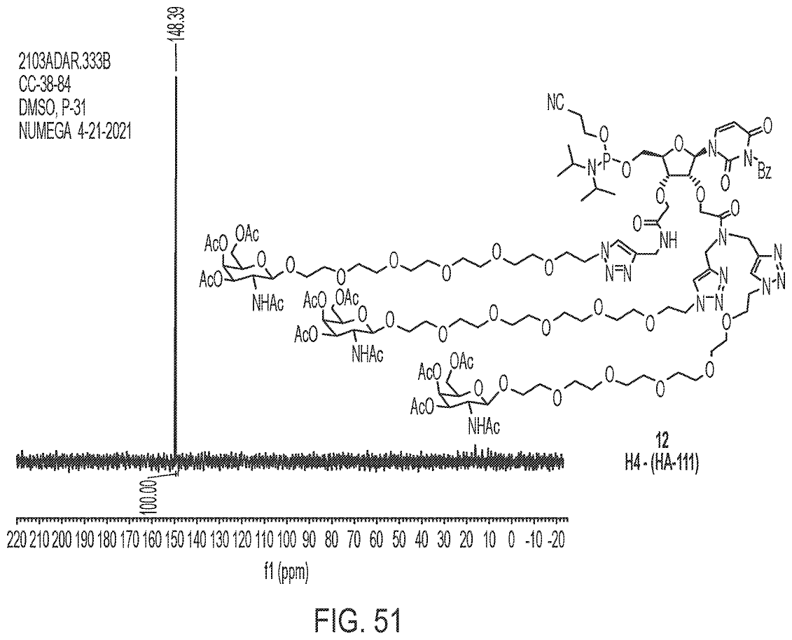

FIG. 52 shows a ¹H NMR spectrum of compound 12 from Example 11.

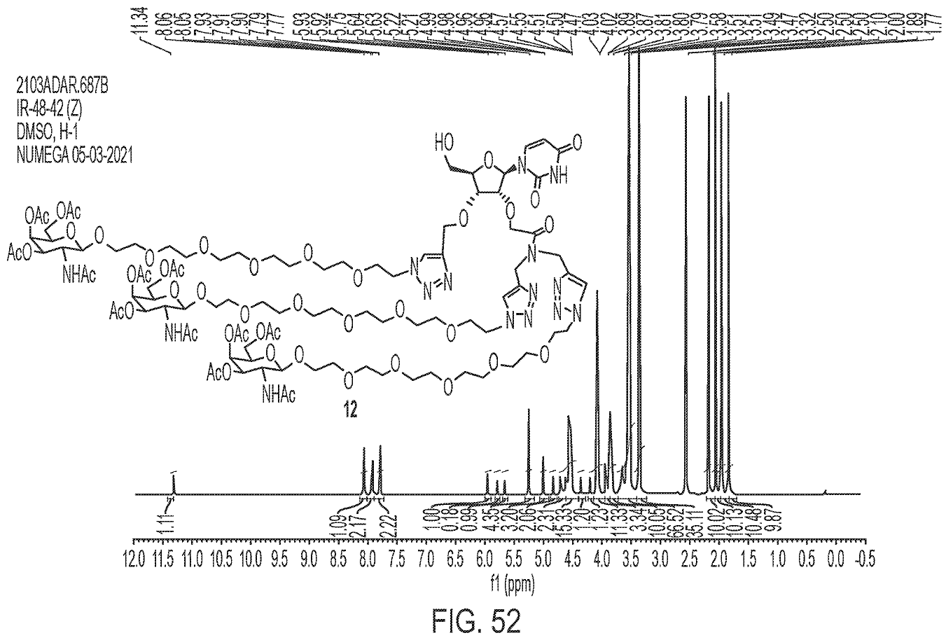

FIG. 53 shows a ¹H NMR spectrum of compound H6-(HA-112) from Example 11.

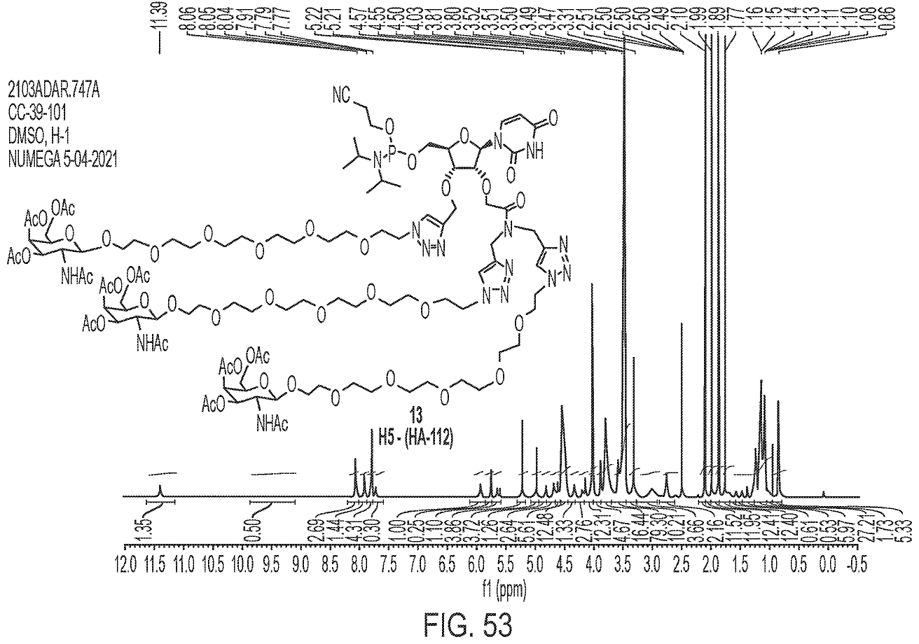

FIG. 54 shows a ³¹P NMR spectrum of compound 116-(HA-112) from Example 11.

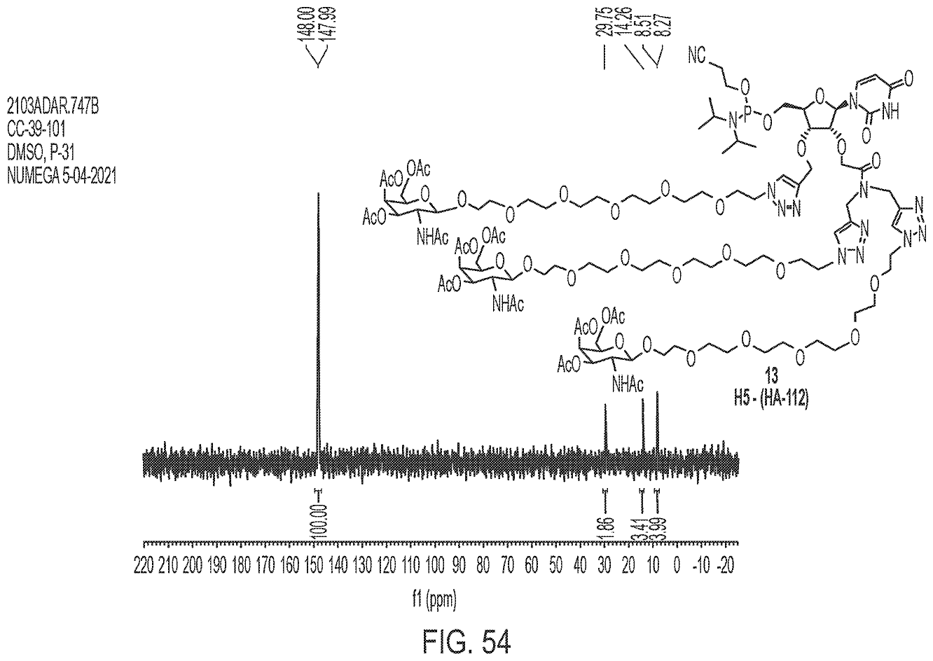

FIG. 55 shows a ¹H NMR spectrum of compound 2 from Example 12.

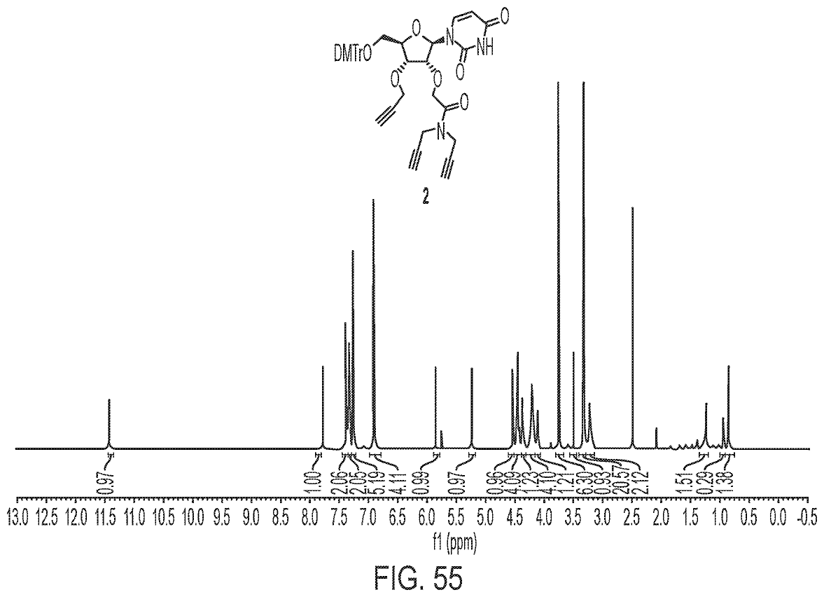

FIG. 56 shows a ¹H NMR spectrum of compound 3 from Example 12.

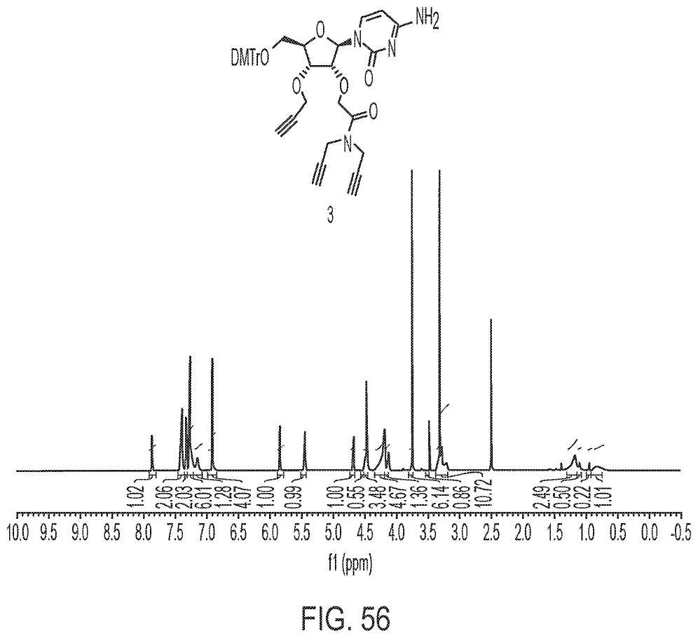

FIG. 57 shows a ¹H NMR spectrum of compound 4 from Example 12.

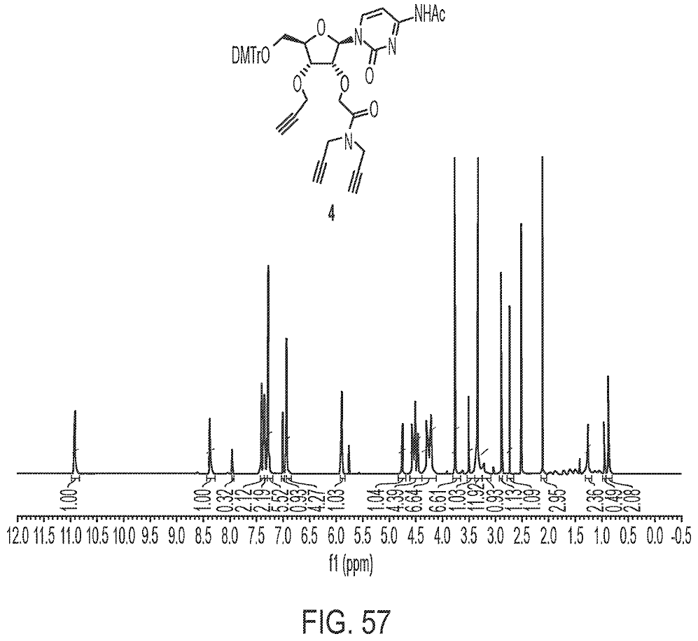

FIG. 58 shows a ¹H NMR spectrum of compound 5 from Example 12.

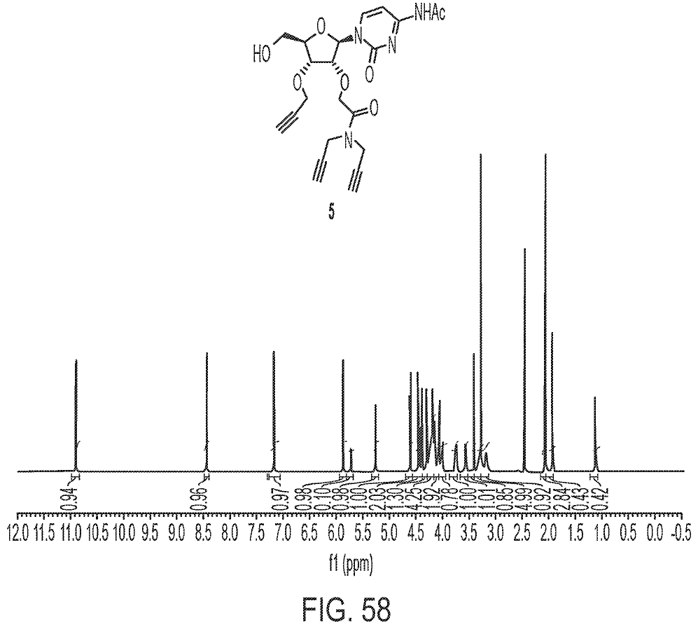

FIG. 59 shows a ¹H NMR spectrum of compound 6 from Example 12.

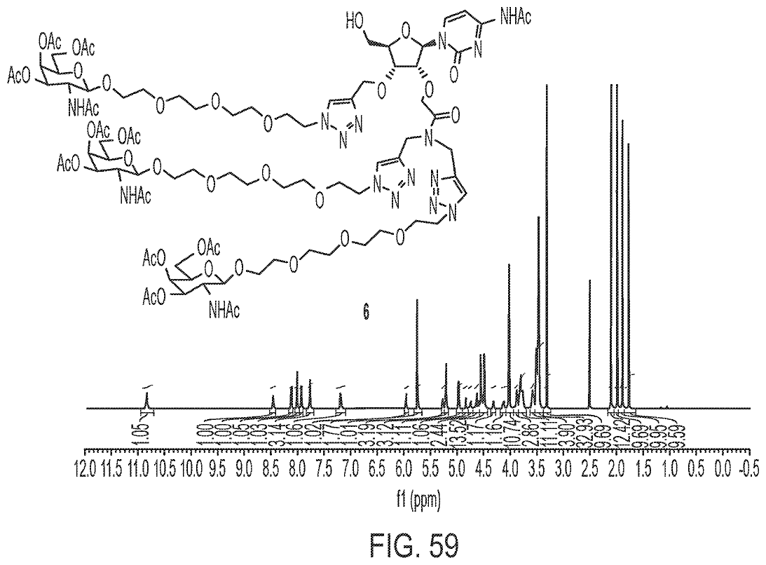

FIG. 60 shows a ¹H NMR spectrum of compound H6-(HA-113) from Example 12.

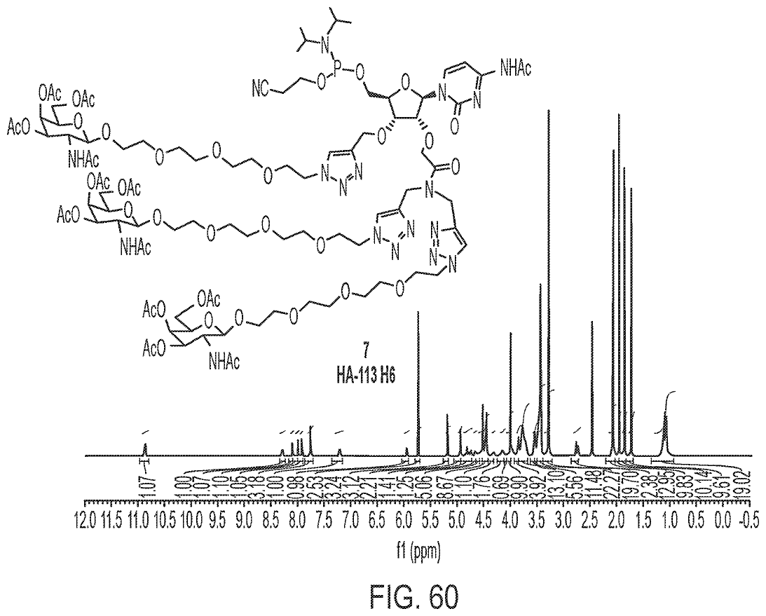

FIG. 61 shows a ³¹P NMR spectrum of compound H6-(HA-113) from Example 12.

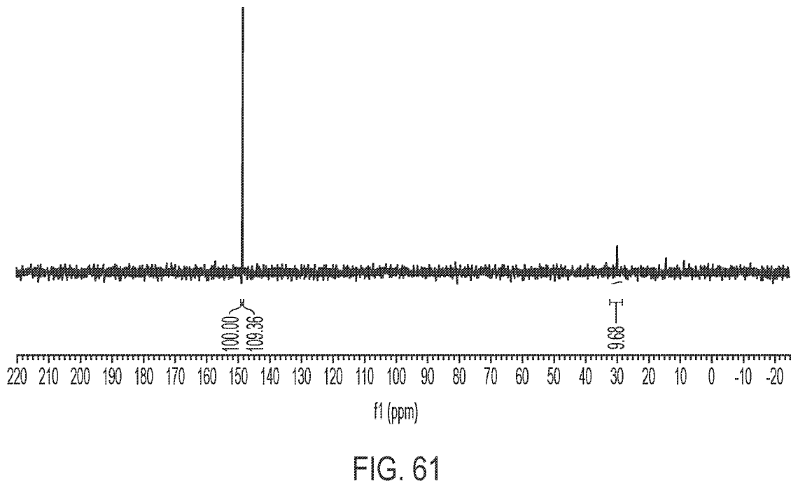

FIG. 62 shows a ¹H NMR spectrum of compound 2 from Example 13.

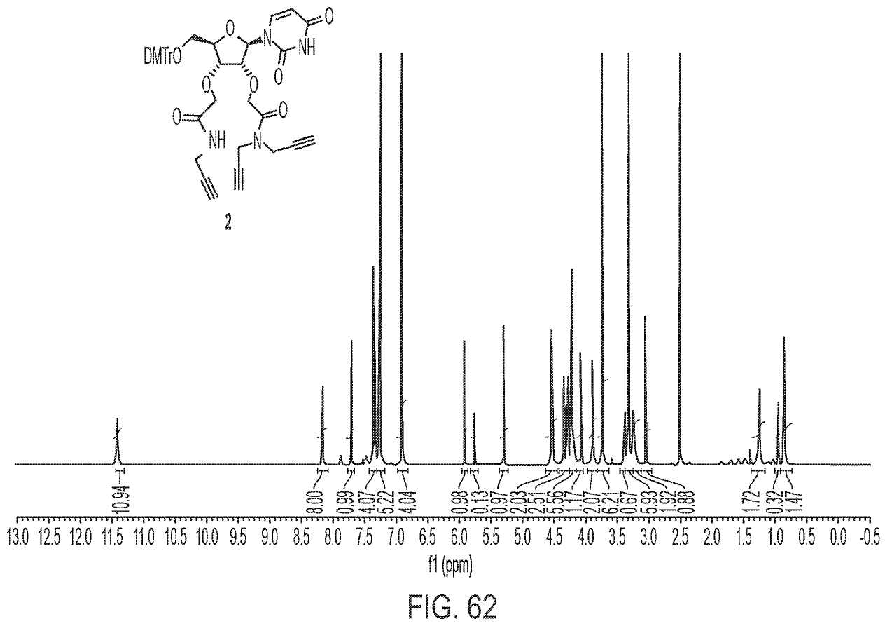

FIG. 63 shows a ¹H NMR spectrum of compound 3 from Example 13.

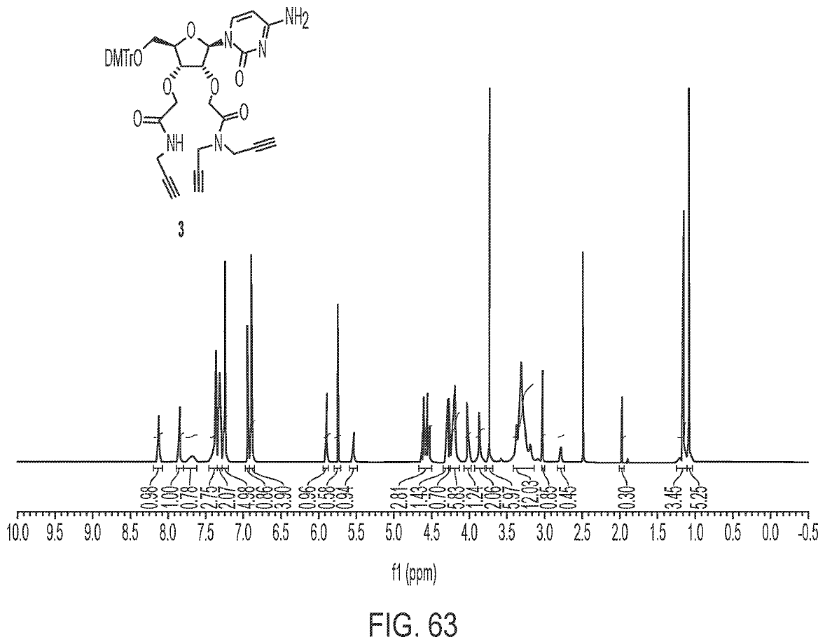

FIG. 64 shows a ¹H NMR spectrum of compound 4 from Example 13.

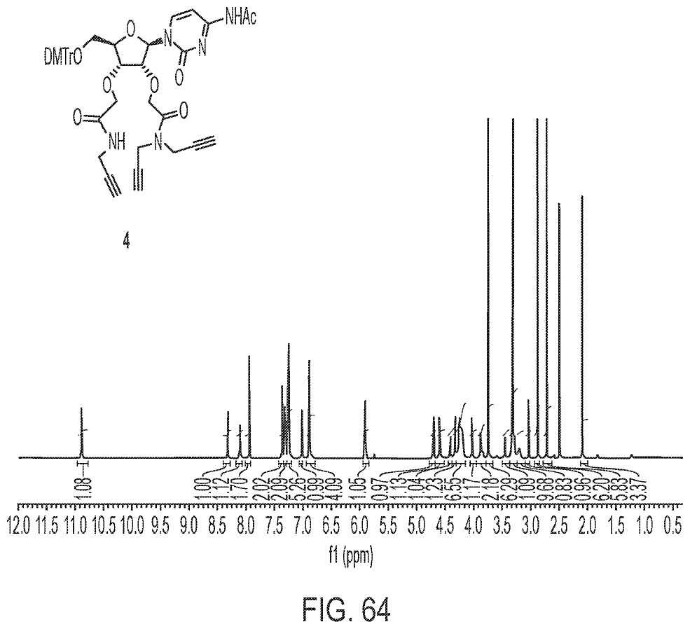

FIG. 65 shows a ¹H NMR spectrum of compound 5 from Example 13.

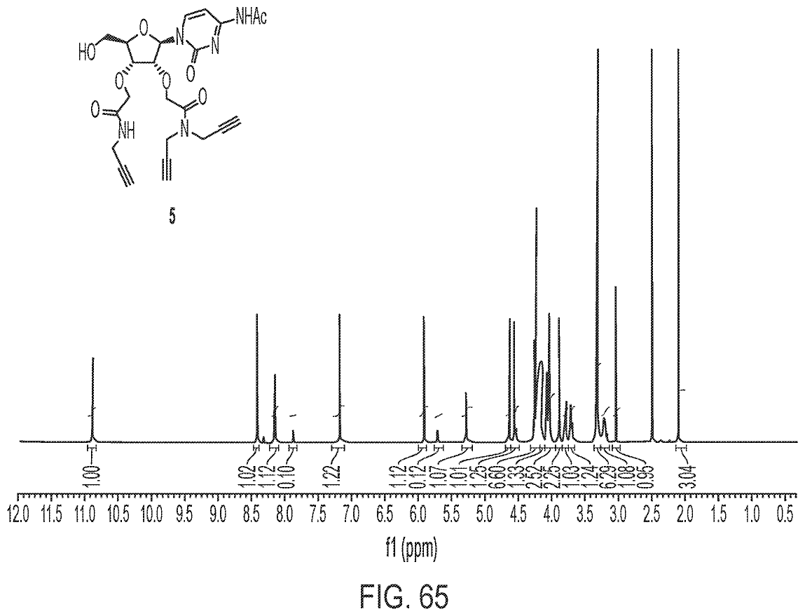

FIG. 66 shows a ¹H NMR spectrum of compound 7 from Example 13.

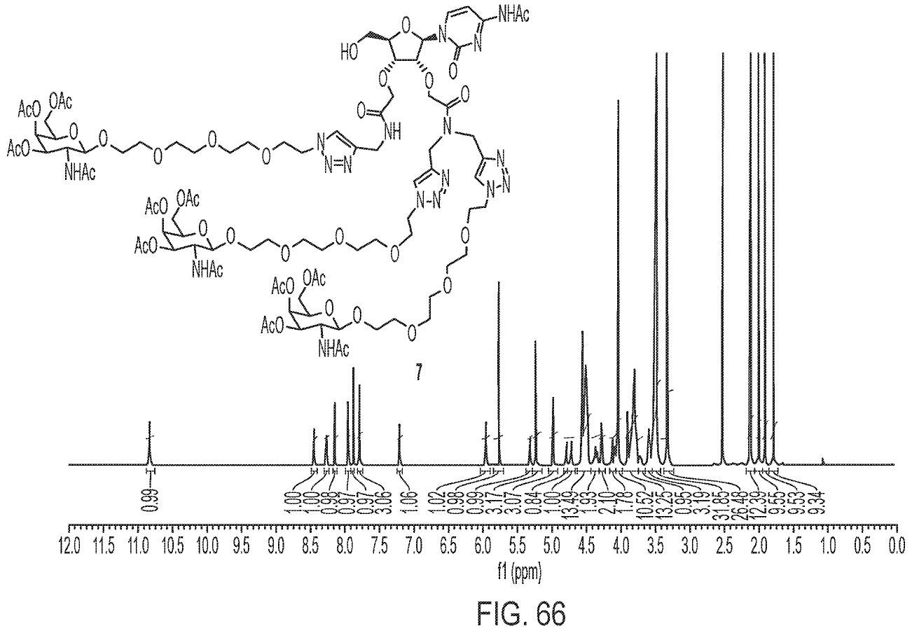

FIG. 67 shows a ¹H NMR spectrum of compound HA-114 from Example 13.

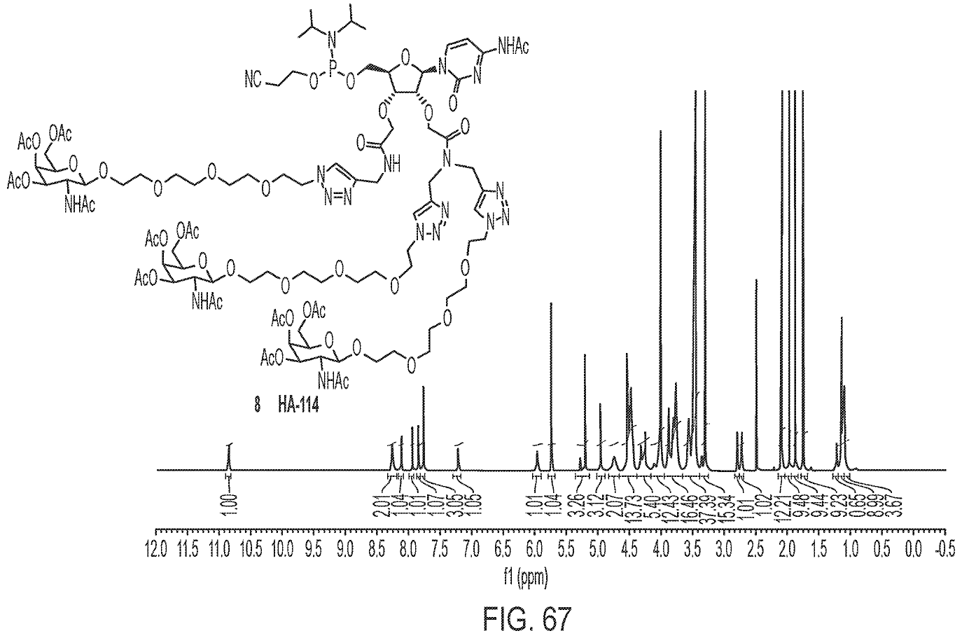

FIG. 68 shows a ³¹P NMR spectrum of compound HA-114 from Example 13.

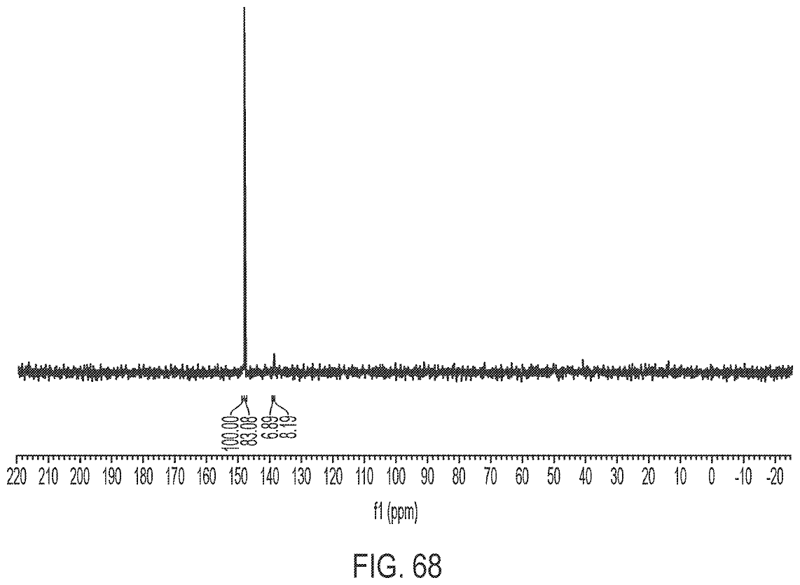

FIG. 69 shows a ¹H NMR spectrum of compound 3 from Example 14.

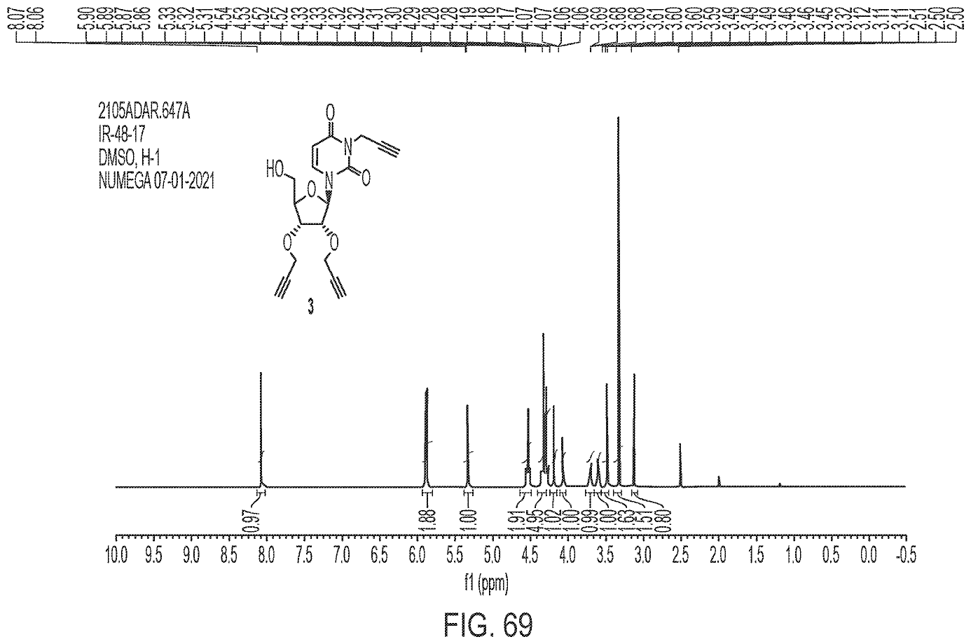

FIG. 70 shows a ¹H NMR spectrum of compound 5 from Example 14.

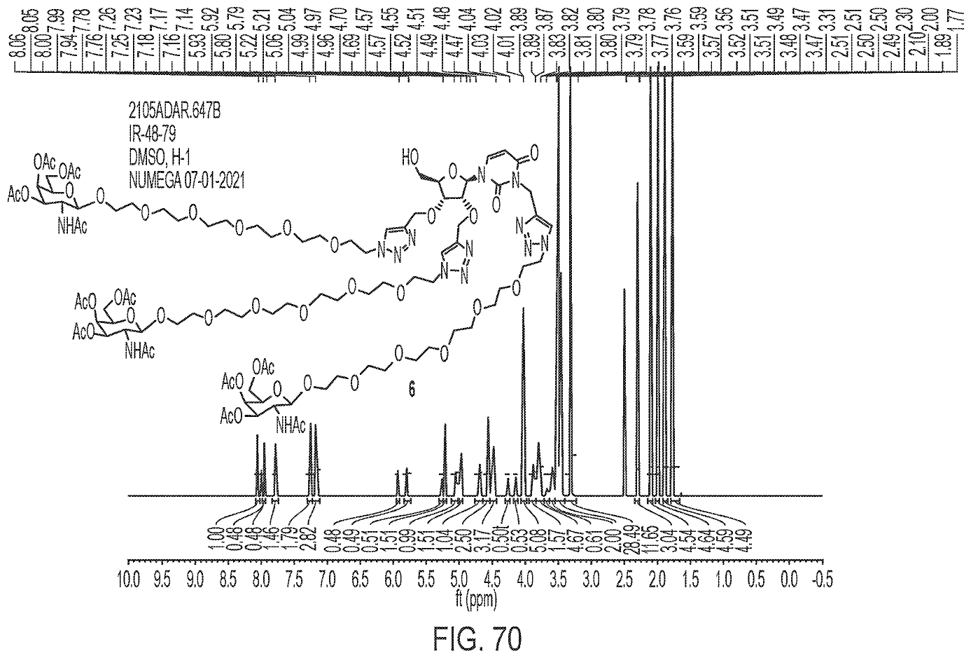

FIG. 71 shows a ¹H NMR spectrum of compound H8-(HA-115) from Example 14.

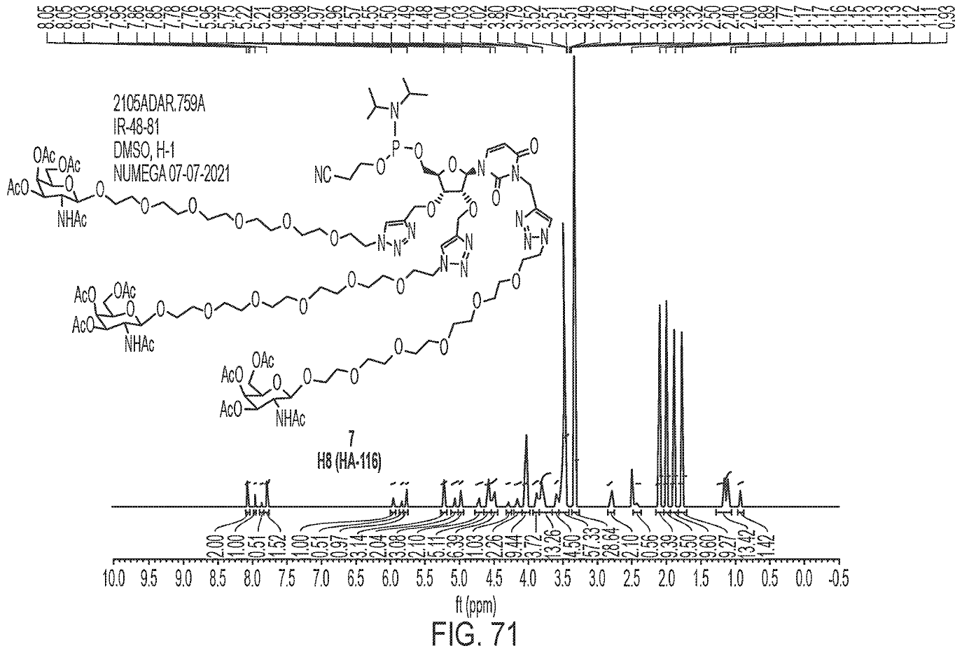

FIG. 72 shows a ³¹P NMR spectrum of compound H8-(HA-115) from Example 14.

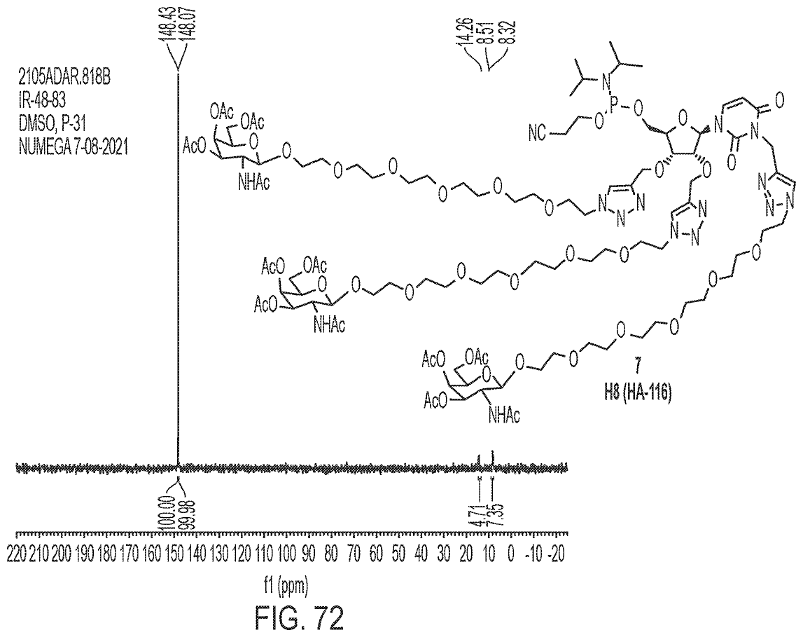

FIG. 73 shows a ¹H NMR spectrum of compound 9 from Example 15.

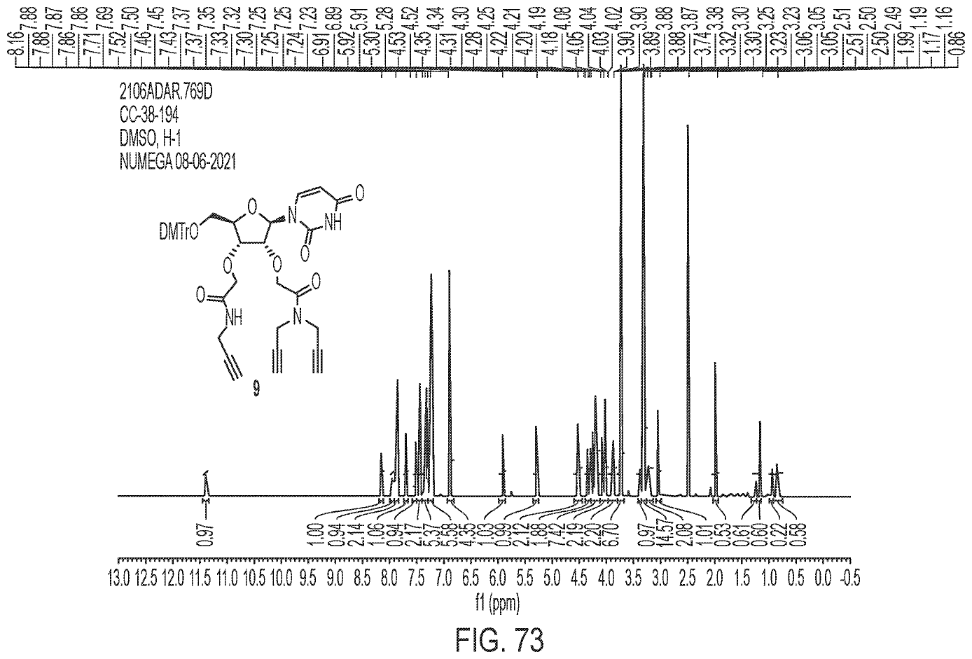

FIG. 74 shows a ¹H NMR spectrum of compound 10 from Example 15.

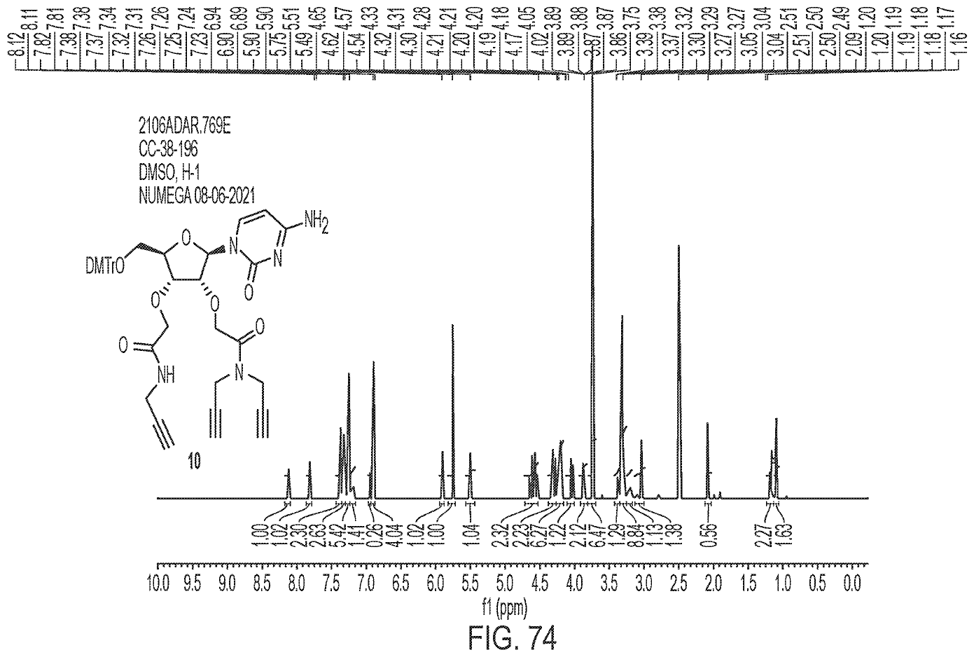

FIG. 75 shows a ¹H NMR spectrum of compound 12 from Example 15.

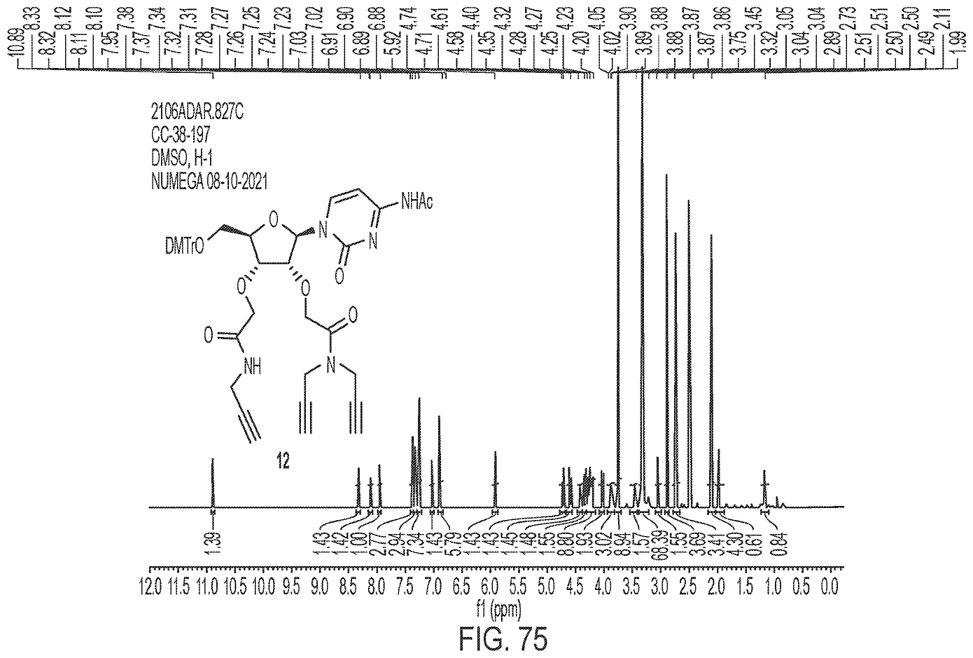

FIG. 76 shows a ¹H NMR spectrum of compound 13 from Example 15.

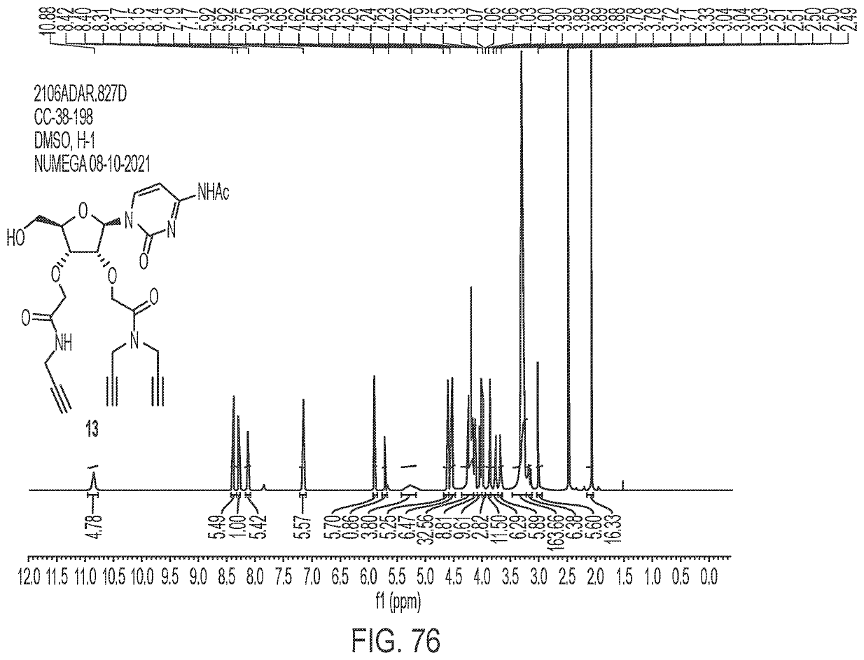

FIG. 77 shows a ¹H NMR spectrum of compound 14 from Example 15.

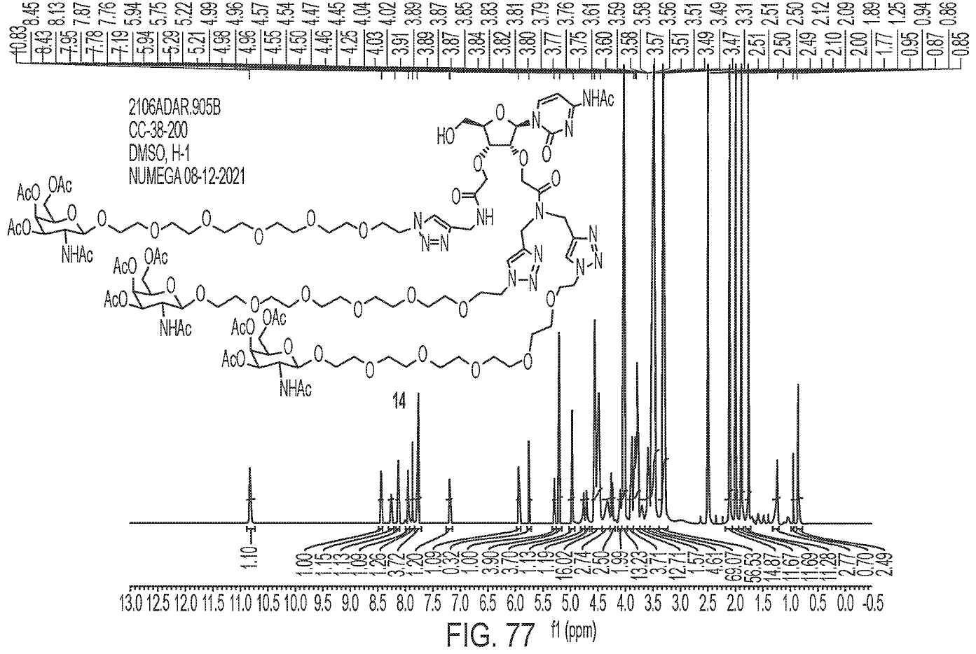

FIG. 78 shows a ¹H NMR spectrum of compound 119-(HA-116) from Example 15.

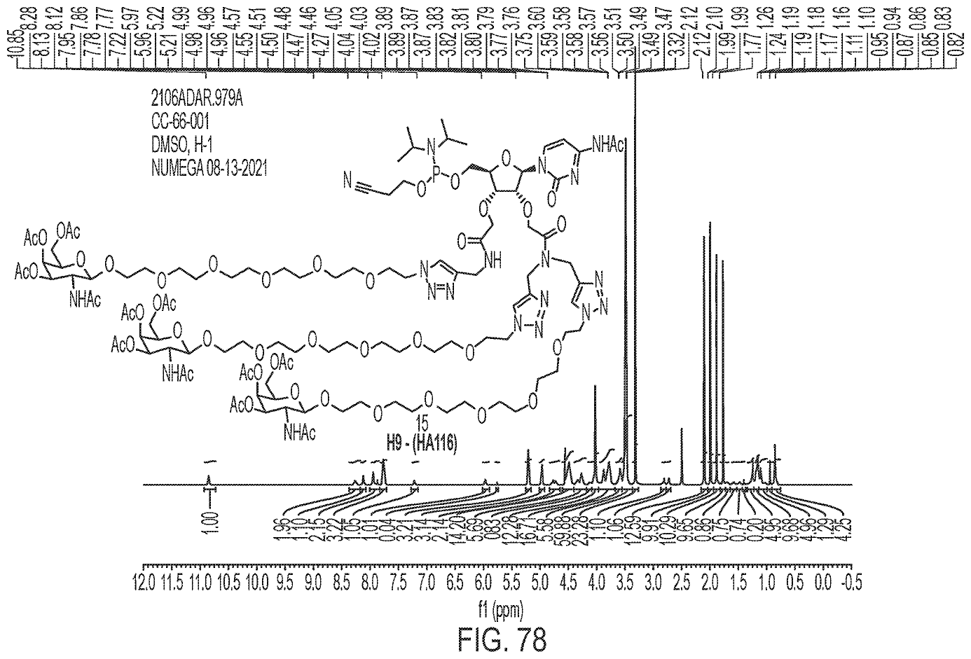

FIG. 79 shows a ³¹P NMR spectrum of compound H9-(HA-116) from Example 15.

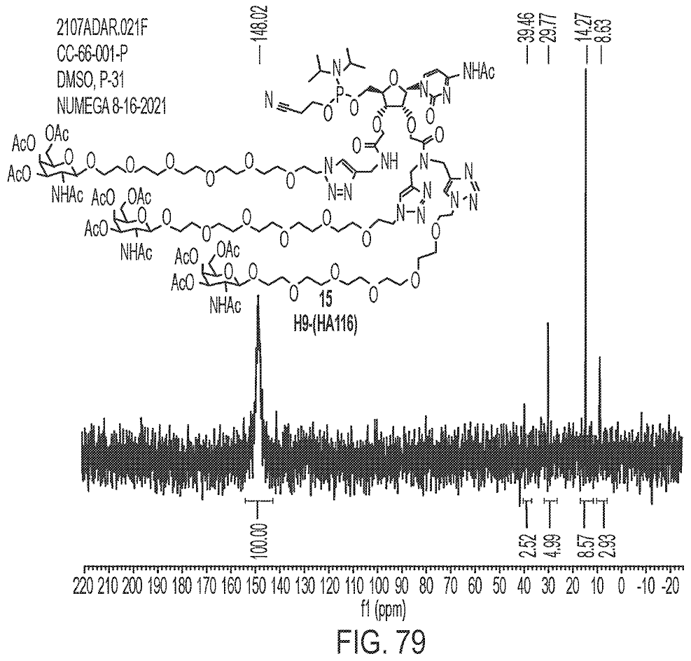

FIG. 80 shows a ¹H NMR spectrum of compound 2 from Example 16.

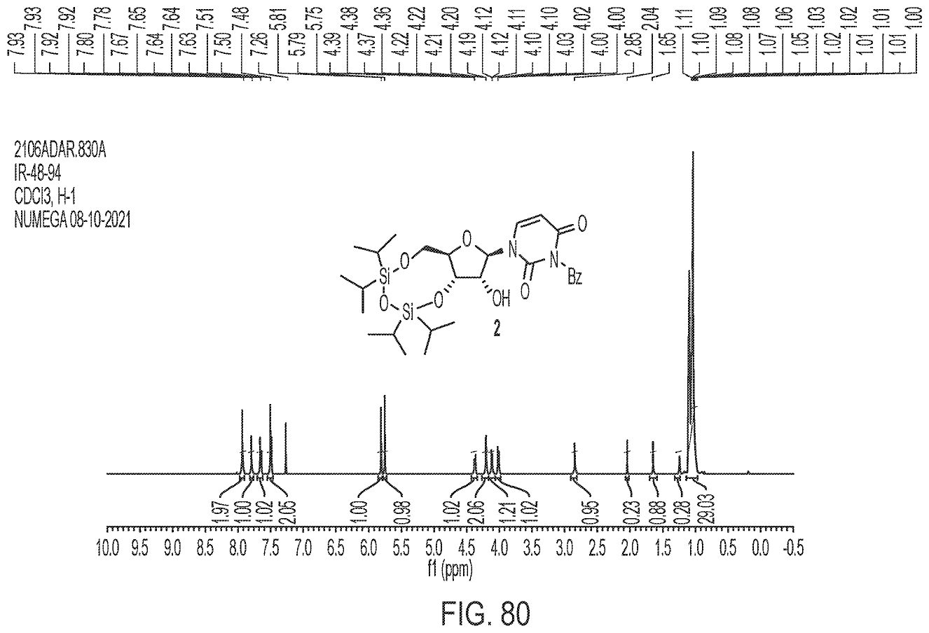

FIG. 81 shows a ¹H NMR spectrum of compound 6 from Example 16.

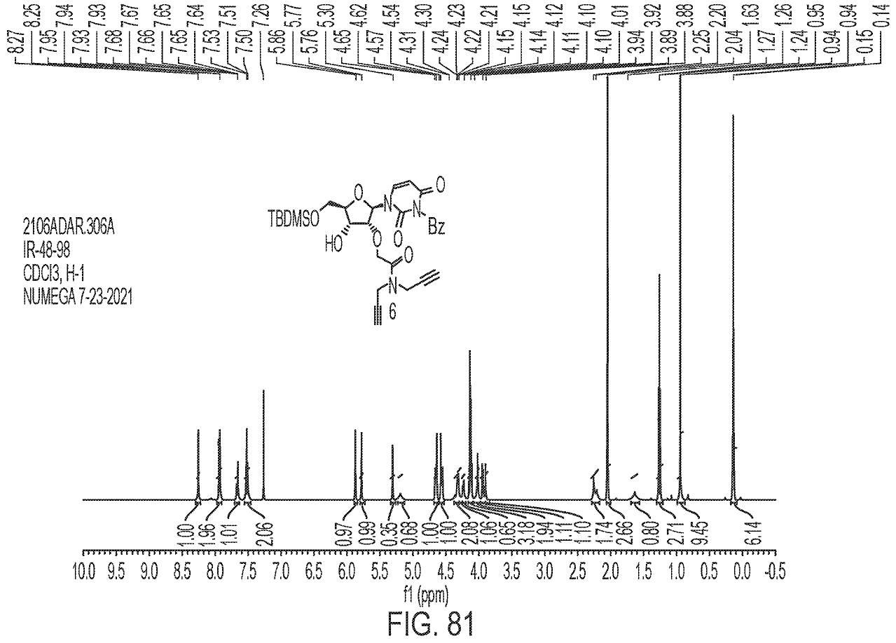

FIG. 82 shows a ¹H NMR spectrum of compound 8 from Example 16.

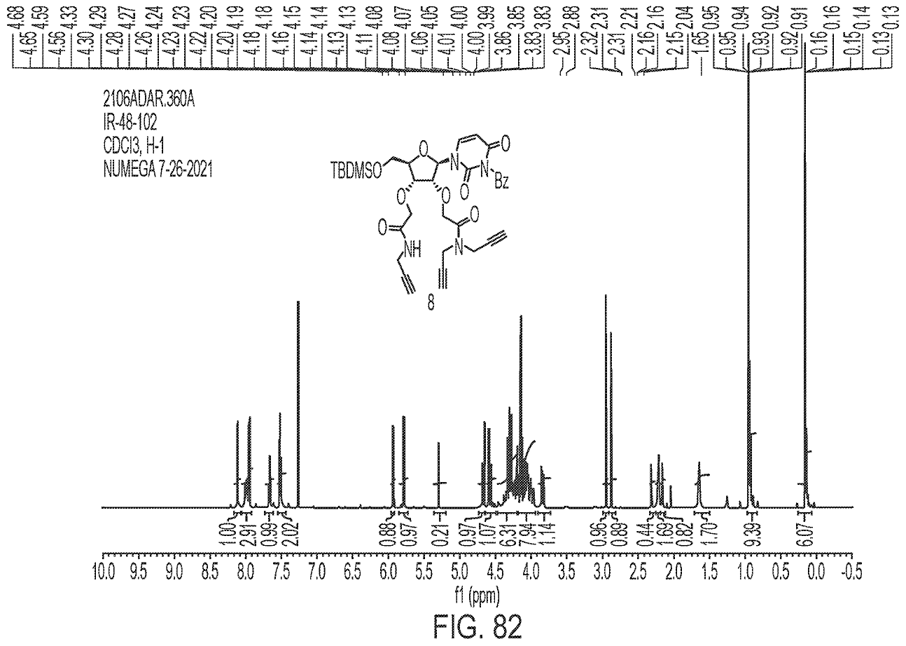

FIG. 83 shows a ¹H NMR spectrum of compound 12 from Example 16.

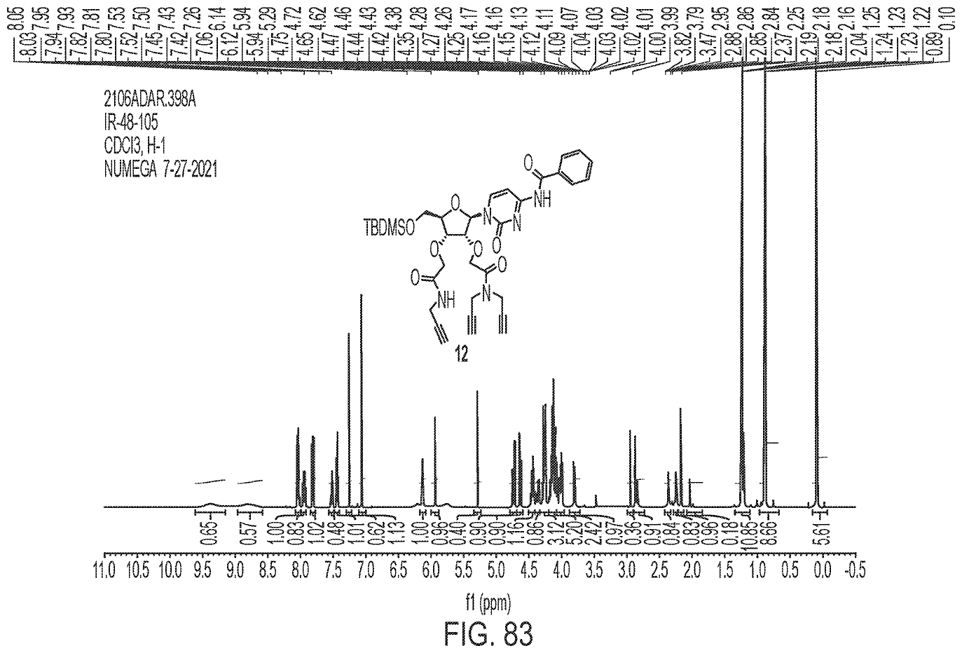

FIG. 84 shows a ¹H NMR spectrum of compound 14 from Example 16.

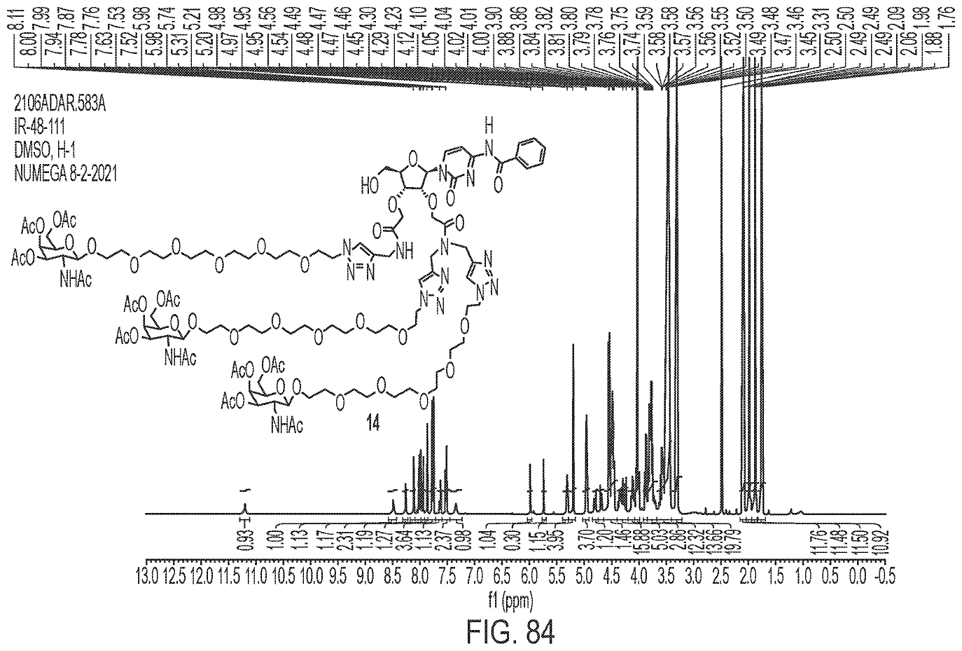

FIG. 85 shows a ¹H NMR spectrum of compound 15 (119-(HA-118)) from Example 16.

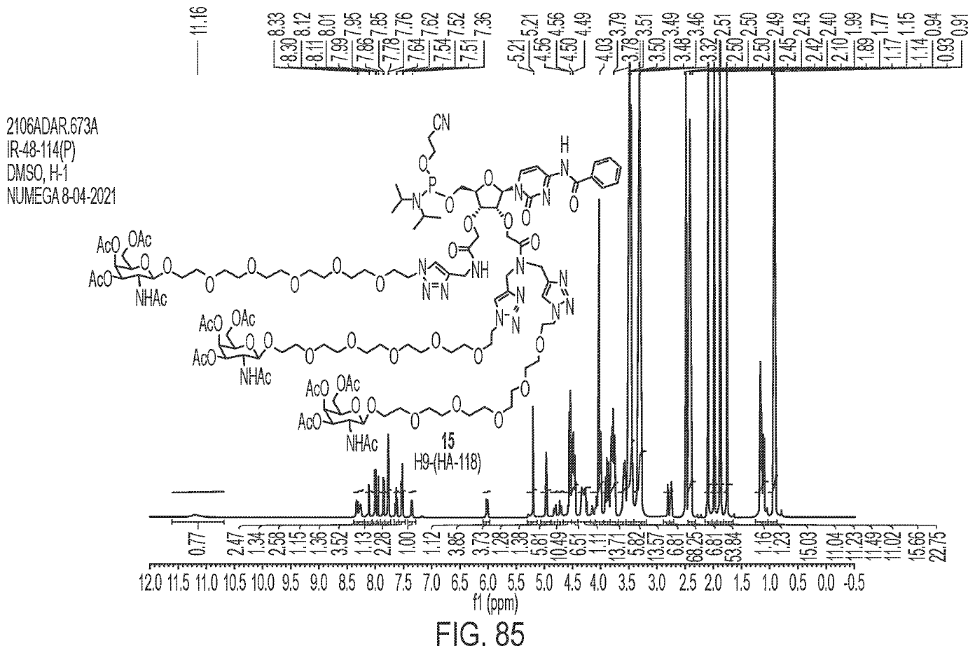

FIG. 86 shows a ³¹P NMR spectrum of compound 15 (H9-(HA-118)) from Example 16.

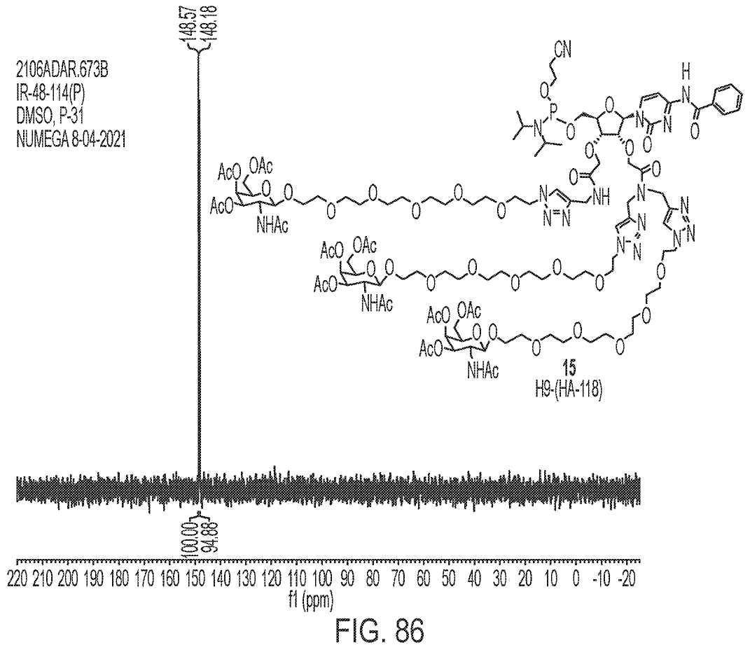

FIG. 87 shows a ¹H NMR spectrum of compound 2 from Example 17.

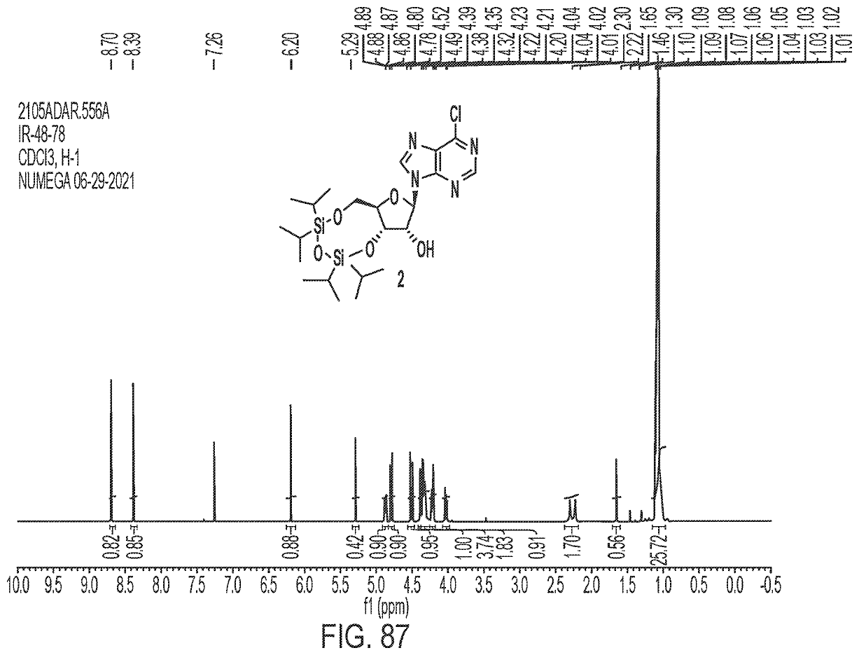

FIG. 88 shows a ¹H NMR spectrum of compound 5 from Example 17.

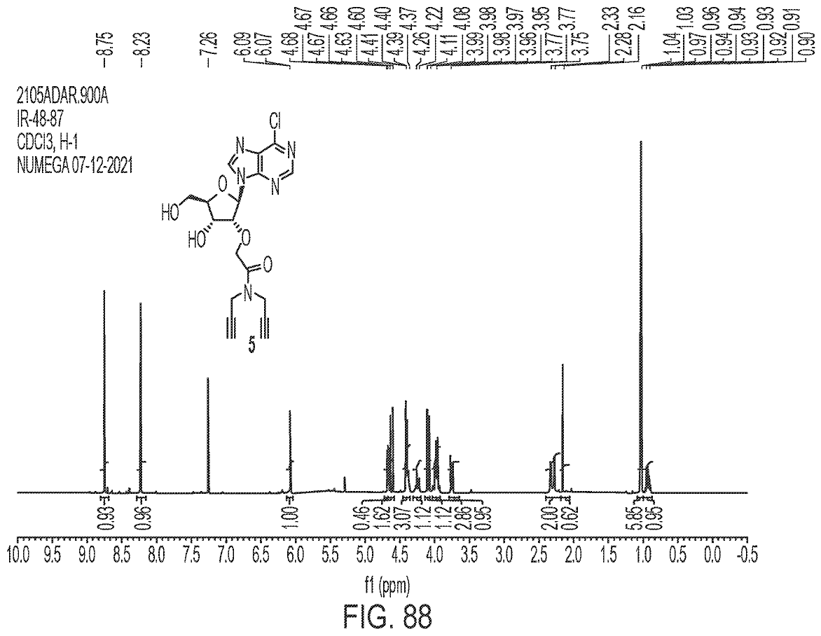

FIG. 89 shows a ¹H NMR spectrum of compound 8 from Example 17.

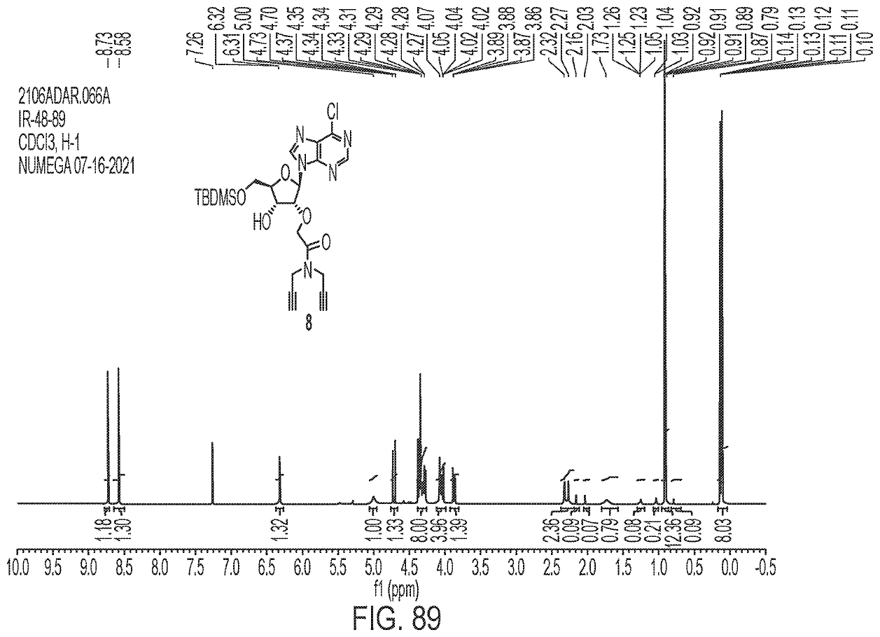

FIG. 90 shows a ¹H NMR spectrum of compound 9 from Example 17.

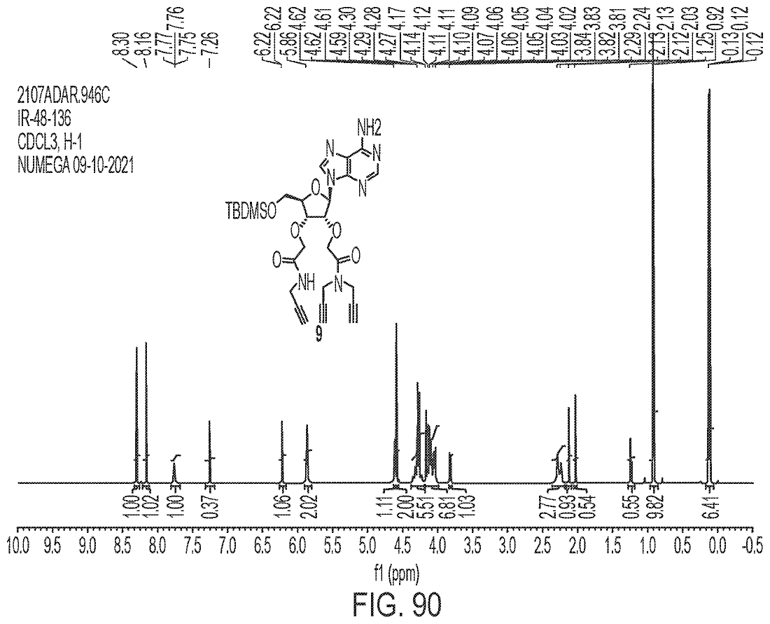

FIG. 91 shows a ¹H NMR spectrum of compound 13 from Example 17.

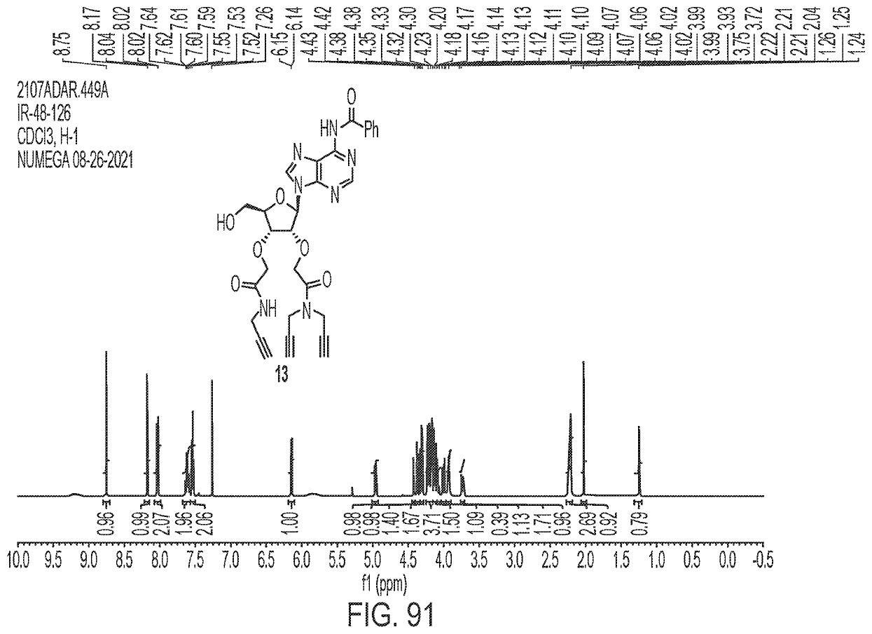

FIG. 92 shows a ¹H NMR spectrum of compound 14 from Example 17.

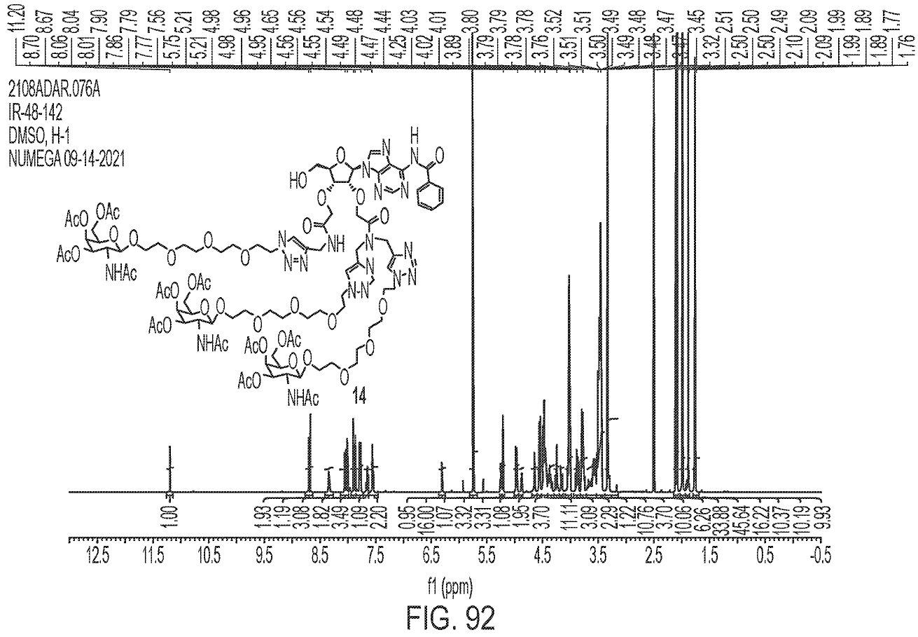

FIG. 93 shows a ¹H NMR spectrum of compound 15 (Hd-(HA-121)) from Example 17.

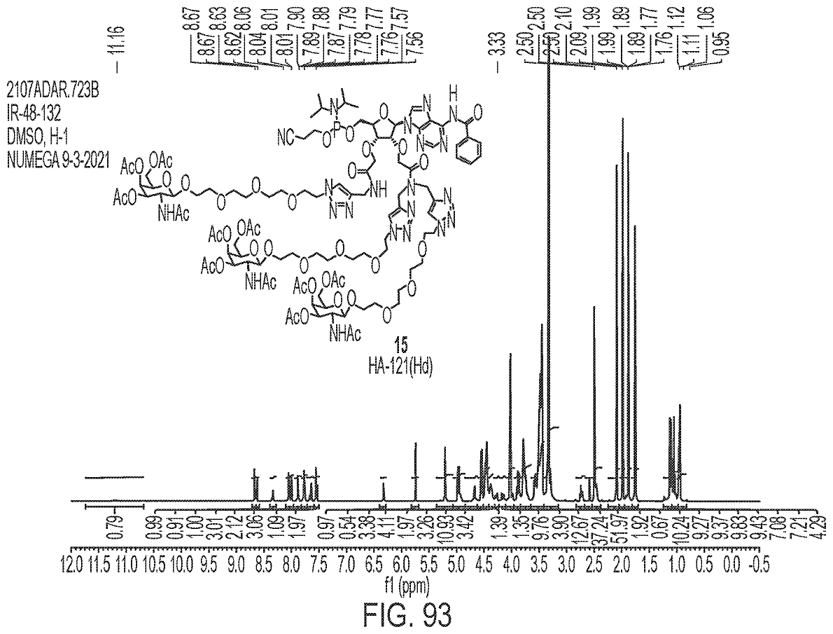

FIG. 94 shows a ³¹P NMR spectrum of compound 15 (Hd-(HA-121)) from Example 17.

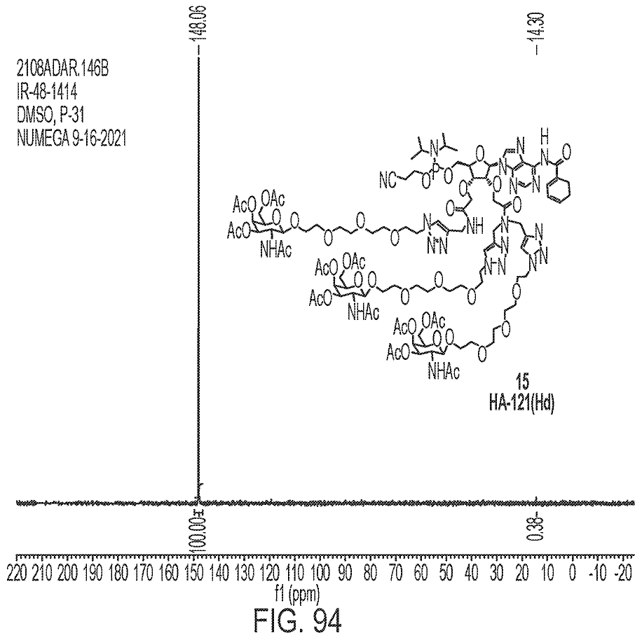

### DETAILED DESCRIPTION

### Definitions

In order that the invention may be more readily understood, certain terms are first defined here for convenience.

As used herein, the term “treating” a disorder encompasses ameliorating, mitigating and/or managing the disorder and/or conditions that may cause the disorder. The terms “treating” and “treatment” refer to a method of alleviating or abating a disease and/or its attendant symptoms. In accordance with the present disclosure, “treating” includes blocking, inhibiting, attenuating, protecting against, modulating, reversing the effects of, and reducing the occurrence of e.g., the harmful effects of a disorder. As used herein, “inhibiting” encompasses preventing, reducing and halting progression.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography (HPLC). Particularly, in certain embodiments the compound or oligonucleotide is at least 85% pure, more preferably at least 90% pure, more preferably at least 95% pure, and most preferably at least 99% pure.

The term “administration” or “administering” includes routes of introducing the compound(s) or oligonucleotide(s) to a subject to perform their intended function. Examples of routes of administration which can be used include injection (subcutaneous, intravenous, parenterally, intraperitoneally, intrathecal), topical, oral, inhalation, rectal and transdermal.

The term “effective amount” includes an amount effective, at dosages and for periods of time necessary, to achieve the desired result. An effective amount of compound or oligonucleotide may vary according to factors such as the disease state, age, and weight of the subject, and the ability of the compound or oligonucleotide to elicit a desired response in the subject. Dosage regimens may be adjusted to provide the optimum therapeutic response. An effective amount is also one in which any toxic or detrimental effects (e.g., side effects) of the inhibitor compound or oligonucleotide are outweighed by the therapeutically beneficial effects.

The phrases “systemic administration,” “administered systemically”, “peripheral administration” and “administered peripherally” as used herein mean the administration of a compound(s), oligonucleotide(s), drug, or other material, such that it enters the patient's system and, thus, is subject to metabolism and other like processes.

The term “therapeutically effective amount” refers to the amount of the compound or oligonucleotide being administered sufficient to prevent development of or alleviate to some extent one or more of the symptoms of the condition or disorder being treated.

A therapeutically effective amount of compound or oligonucleotide (i.e., an effective dosage) may range from about 0.005 μg/kg to about 200 mg/kg, preferably about 0.01 mg/kg to about 200 mg/kg, more preferably about 0.015 mg/kg to about 30 mg/kg of body weight. In other embodiments, the therapeutically effect amount may range from about 1.0 pM to about 10 μM. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a compound or oligonucleotide can include a single treatment or, preferably, can include a series of treatments. In one example, a subject is treated with a compound or oligonucleotide in the range of between about 0.005 μg/kg to about 200 mg/kg of body weight, one time per day for between about 1 to 10 weeks, preferably between 2 to 8 weeks, more preferably between about 3 to 7 weeks, and even more preferably for about 4, 5, or 6 weeks. In another example, a subject may be treated daily for several years in the setting of a chronic condition or illness. It will also be appreciated that the effective dosage of a compound or oligonucleotide used for treatment may increase or decrease over the course of a particular treatment.

The term “chiral” refers to molecules which have the property of non-superimposability of the mirror image partner, while the term “achiral” refers to molecules which are superimposable on their mirror image partner.

The term “diastereomers” refers to stereoisomers with two or more centers of dissymmetry and whose molecules are not mirror images of one another.

The term “enantiomers” refers to two stereoisomers of a compound which are non-superimposable mirror images of one another. An equimolar mixture of two enantiomers is called a “racemic mixture” or a “racemate.”

The term “isomers” or “stereoisomers” refers to compounds which have identical chemical constitution but differ with regard to the arrangement of the atoms or groups in space.

The term “prodrug” includes compounds with moieties which can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branched or unbranched lower alkyl ester moieties, (e.g., propionic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionic acid esters and acyl esters. Prodrugs which are converted to active forms through other mechanisms in vivo are also included. In aspects, the compounds of the present disclosure are prodrugs of any of the formulae herein.

The term “subject” refers to animals such as mammals, including, but not limited to, primates (e.g., humans), cows, sheep, goats, horses, dogs, cats, rabbits, rats, mice and the like. In certain embodiments, the subject is a human.

The terms “a,” “an,” and “the” refer to “one or more” when used in this application, including the claims. Thus, for example, reference to “a sample” includes a plurality of samples, unless the context clearly is to the contrary (e.g., a plurality of samples), and so forth.

Throughout this specification and the claims, the words “comprise,” “comprises,” and “comprising” are used in a non-exclusive sense, except where the context requires otherwise.

As used herein, the term “about,” when referring to a value is meant to encompass variations of, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

As used herein, the term “alkyl” refers to a straight-chained or branched hydrocarbon group containing 1 to 20 carbon atoms. A specified number of carbon atoms within this range includes for example, C1-C12 alkyl (having 1-12 carbon atoms) and C1-C4 alkyl (having 1-4 carbon atoms). The term “lower alkyl” refers to a C1-C6 alkyl chain. Examples of alkyl groups include methyl, ethyl, n-propyl, isopropyl, tert-butyl, and n-pentyl. Alkyl groups may be optionally substituted with one or more substituents.

The term “haloalkyl” refers to an alkyl group that is substituted by one or more halo substituents. Examples of haloalkyl groups include fluoromethyl, difluoromethyl, trifluoromethyl, bromomethyl, chloromethyl, and 2,2,2-trifluoroethyl.

The term “alkenyl” refers to an unsaturated hydrocarbon chain that may be a straight chain or branched chain, containing 2 to 12 carbon atoms and at least one carbon-carbon double bond. Alkenyl groups may be optionally substituted with one or more substituents.

The term “arylalkenyl” refers to an unsaturated hydrocarbon chain that may be a straight chain or branched chain, containing 2 to 12 carbon atoms and at least one carbon-carbon double bond wherein one or more of the sp² hybridized carbons of the alkenyl unit attaches to an aryl moiety. Alkenyl groups may be optionally substituted with one or more substituents.

The term “alkynyl” refers to an unsaturated hydrocarbon chain that may be a straight chain or branched chain, containing the 2 to 12 carbon atoms and at least one carbon-carbon triple bond. Alkynyl groups may be optionally substituted with one or more substituents.

The term “arylalkynyl” refers to an unsaturated hydrocarbon chain that may be a straight chain or branched chain, containing 2 to 12 carbon atoms and at least one carbon-carbon triple bond wherein one or more of the sp hybridized carbons of the alkynyl unit attaches to an aryl moiety. Alkynyl groups may be optionally substituted with one or more substituents.

The sp² or sp carbons of an alkenyl group and an alkynyl group, respectively, may optionally be the point of attachment of the alkenyl or alkynyl groups.

The term “alkoxy” refers to an —O-alkyl substituent.

As used herein, the term “halogen”, “hal” or “halo” means —F, —Cl, —Br or —I.

The term “alkylthio” refers to an —S-alkyl substituent.

The term “alkoxyalkyl” refers to an -alkyl-O-alkyl substituent.

The term “haloalkoxy” refers to an —O-alkyl that is substituted by one or more halo substituents. Examples of haloalkoxy groups include trifluoromethoxy, and 2,2,2-trifluoroethoxy.

The term “haloalkoxyalkyl” refers to an -alkyl-O-alkyl’ where the alkyl’ is substituted by one or more halo substituents.

The term “haloalkylaminocarbonyl” refers to a —C(O)-amino-alkyl where the alkyl is substituted by one or more halo substituents.

The term “haloalkylthio” refers to an —S-alkyl that is substituted by one or more halo substituents. Examples of haloalkylthio groups include trifluoromethylthio, and 2,2,2-trifluoroethylthio.

The term “haloalkylcarbonyl” refers to an —C(O)-alkyl that is substituted by one or more halo substituents. An example of a haloalkylcarbonyl group includes trifluoroacetyl.

The term “cycloalkyl” refers to a hydrocarbon 3-8 membered monocyclic or 7-14 membered bicyclic ring system having at least one saturated ring or having at least one non-aromatic ring, wherein the non-aromatic ring may have some degree of unsaturation. Cycloalkyl groups may be optionally substituted with one or more substituents. In one embodiment, 0, 1, 2, 3, or 4 atoms of each ring of a cycloalkyl group may be substituted by a substituent. Representative examples of cycloalkyl group include cyclopropyl, cyclopentyl, cyclohexyl, cyclobutyl, cycloheptyl, cyclopentenyl, cyclopentadienyl, cyclohexenyl, cyclohexadienyl, and the like.

The term “cycloalkoxy” refers to an —O-cycloalkyl substituent.

The term “cycloalkoxyalkyl” refers to an -alkyl-O-cycloalkyl substituent.

The term “cycloalkylalkoxy” refers to an —O-alkyl-cycloalkyl substituent.

The term “cycloalkylaminocarbonyl” refers to an —C(O)—NH-cycloalkyl substituent.

The term “aryl” refers to a hydrocarbon monocyclic, bicyclic or tricyclic aromatic ring system. Aryl groups may be optionally substituted with one or more substituents. In one embodiment, 0, 1, 2, 3, 4, 5 or 6 atoms of each ring of an aryl group may be substituted by a substituent. Examples of aryl groups include phenyl, naphthyl, anthracenyl, fluorenyl, indenyl, azulenyl, and the like.

The term “aryloxy” refers to an —O-aryl substituent.

The term “arylalkoxy” refers to an —O-alkyl-aryl substituent.

The term “arylalkylthio” refers to an —S-alkyl-aryl substituent.

The term “arylthioalkyl” refers to an -alkyl-S-aryl substituent.

The term “arylalkylaminocarbonyl” refers to a —C(O)-amino-alkyl-aryl substituent.

The term “arylalkylsulfonyl” refers to an —S(O)₂-alkyl-aryl substituent.

The term “arylalkylsulfinyl” refers to an —S(O)-alkyl-aryl substituent.

The term “aryloxyalkyl” refers to an -alkyl-O-aryl substituent.

The term “alkylaryl” refers to an -aryl-alkyl substituent.

The term “arylalkyl” refers to an -alkyl-aryl substituent.

The term “heteroaryl” refers to an aromatic 5-8 membered monocyclic, 8-12 membered bicyclic, or 11-14 membered tricyclic ring system having 1-4 ring heteroatoms if monocyclic, 1-6 heteroatoms if bicyclic, or 1-9 heteroatoms if tricyclic, said heteroatoms selected from O, N, or S, and the remainder ring atoms being carbon (with appropriate hydrogen atoms unless otherwise indicated). Heteroaryl groups may be optionally substituted with one or more substituents. In one embodiment, 0, 1, 2, 3, or 4 atoms of each ring of a heteroaryl group may be substituted by a substituent. Examples of heteroaryl groups include pyridyl, furanyl, thienyl, pyrrolyl, oxazolyl, oxadiazolyl, imidazolyl thiazolyl, isoxazolyl, quinolinyl, pyrazolyl, isothiazolyl, pyridazinyl, pyrimidinyl, pyrazinyl, triazinyl, isoquinolinyl, indazolyl, and the like.

The term “heteroarylalkyl” refers to an -alkyl-heteroaryl substituent.

The term “heteroaryloxy” refers to an —O-heteroaryl substituent.

The term “heteroarylalkoxy” refers to an —O-alkyl-heteroaryl substituent.

The term “heteroaryloxyalkyl” refers to an -alkyl-O-heteroaryl substituent.

The term “nitrogen-containing heteroaryl” refers to a heteroaryl group having 1-4 ring nitrogen heteroatoms if monocyclic, 1-6 ring nitrogen heteroatoms if bicyclic, or 1-9 ring nitrogen heteroatoms if tricyclic.

The term “heterocycloalkyl” refers to a nonaromatic 3-8 membered monocyclic, 7-12 membered bicyclic, or 10-14 membered tricyclic ring system comprising 1-3 heteroatoms if monocyclic, 1-6 heteroatoms if bicyclic, or 1-9 heteroatoms if tricyclic, said heteroatoms selected from O, N, S, B, P or Si, wherein the nonaromatic ring system is completely saturated. Heterocycloalkyl groups may be optionally substituted with one or more substituents. In one embodiment, 0, 1, 2, 3, or 4 atoms of each ring of a heterocycloalkyl group may be substituted by a substituent. Representative heterocycloalkyl groups include piperidinyl, piperazinyl, tetrahydropyranyl, morpholinyl, thiomorpholinyl, 1,3-dioxolane, tetrahydrofuranyl, tetrahydrothienyl, thiiranyl, and the like.

The term “heterocycloalkylalkyl” refers to an -alkyl-heterocycloalkyl substituent.

The term “alkylamino” refers to an amino substituent which is further substituted with one or two alkyl groups. The term “aminoalkyl” refers to an alkyl substituent which is further substituted with one or more amino groups. The term “hydroxyalkyl” or “hydroxylalkyl” refers to an alkyl substituent which is further substituted with one or more hydroxyl groups. The alkyl or aryl portion of alkylamino, aminoalkyl, mercaptoalkyl, hydroxyalkyl, mercaptoalkoxy, sulfonylalkyl, sulfonylaryl, alkylcarbonyl, and alkylcarbonylalkyl may be optionally substituted with one or more substituents.

The term “nucleobase” refers to nitrogen-containing biological compounds that form nucleosides. They include purine bases and pyrimidine bases. Five nucleobases—adenine (A), cytosine (C), guanine (G), thymine (T), and uracil (U)—are referred to as primary or canonical nucleobases. When a nucleobase is listed in a formula definition, it refers to that moiety covalently bonded to the recited formula.

The term “modified nucleobase” refers to derivatives of a nucleobase. Examples of modified nucleobases include, but are not limited to, xanthine, hypoxanthine, 7-methylguanine, 5,6-dihydrouracil, 5-methylcytosine, 5-hydroxymethylcytosine, purine, 2,6-diaminopurine, and 6,8-diaminopurine. When a modified nucleobase is listed in a formula definition, it refers to that moiety covalently bonded to the recited formula.

Acids and bases useful in the methods herein are known in the art. Acid catalysts are any acidic chemical, which can be inorganic (e.g., hydrochloric, sulfuric, nitric acids, aluminum trichloride) or organic (e.g., camphorsulfonic acid, p-toluenesulfonic acid, acetic acid, ytterbium triflate) in nature. Acids are useful in either catalytic or stoichiometric amounts to facilitate chemical reactions. Bases are any basic chemical, which can be inorganic (e.g., sodium bicarbonate, potassium hydroxide) or organic (e.g., triethylamine, pyridine) in nature. Bases are useful in either catalytic or stoichiometric amounts to facilitate chemical reactions.

Alkylating agents are any reagent that is capable of effecting the allylation of the functional group at issue (e.g., oxygen atom of an alcohol, nitrogen atom of an amino group). Alkylating agents are known in the art, including in the references cited herein, and include alkyl halides (e.g., methyl iodide, benzyl bromide or chloride), alkyl sulfates (e.g., methyl sulfate), or other alkyl group-leaving group combinations known in the art. Leaving groups are any stable species that can detach from a molecule during a reaction (e.g., elimination reaction, substitution reaction) and are known in the art, including in the references cited herein, and include halides (e.g., I—, Cl—, Br—, F—), hydroxy, alkoxy (e.g., —OMe, —O-t-Bu), acyloxy anions (e.g., —OAc, —OC(O)CF₃), sulfonates (e.g., mesyl, tosyl), acetamides (e.g., —NHC(O)Me), carbamates (e.g., N(Me)C(O)Ot-Bu), phosphonates (e.g., —OP(O)(OEt)₂), water or alcohols (protic conditions), and the like.

In certain embodiments, substituents on any group (such as, for example, alkyl, alkenyl, alkynyl, aryl, aralkyl, heteroaryl, heteroaralkyl, cycloalkyl, heterocycloalkyl) can be at any atom of that group, wherein any group that can be substituted (such as, for example, alkyl, alkenyl, alkynyl, aryl, aralkyl, heteroaryl, heteroaralkyl, cycloalkyl, heterocycloalkyl) can be optionally substituted with one or more substituents (which may be the same or different), each replacing a hydrogen atom. Examples of suitable substituents include, but are not limited to alkyl, alkenyl, alkynyl, cycloalkyl, heterocycloalkyl, aralkyl, heteroaralkyl, aryl, heteroaryl, halogen, haloalkyl, cyano, nitro, alkoxy, aryloxy, hydroxyl, hydroxylalkyl, oxo (i.e., carbonyl), carboxyl, formyl, alkylcarbonyl, alkylcarbonylalkyl, alkoxycarbonyl, alkylcarbonyloxy, aryloxycarbonyl, heteroaryloxy, heteroaryloxycarbonyl, thio, mercapto, mercaptoalkyl, arylsulfonyl, amino, aminoalkyl, dialkylamino, alkylcarbonylamino, alkylaminocarbonyl, alkoxycarbonylamino, alkylamino, arylamino, diarylamino, alkylcarbonyl, or arylamino-substituted aryl; arylalkylamino, aralkylaminocarbonyl, amido, alkylaminosulfonyl, arylaminosulfonyl, dialkylaminosulfonyl, alkylsulfonylamino, arylsulfonylamino, imino, carboxamido, carbamido, carbamyl, thioureido, thiocyanato, sulfoamido, sulfonylalkyl, sulfonylaryl, mercaptoalkoxy, N-hydroxyamidinyl, or N′-aryl, N″-hydroxyamidinyl. In certain embodiments, substituents on any group include alkyl, alkenyl, alkynyl, cycloalkyl, heterocycloalkyl, aralkyl, heteroaralkyl, aryl, heteroaryl, halogen, haloalkyl, cyano, nitro, alkoxy, aryloxy, hydroxyl, hydroxylalkyl, oxo (i.e., carbonyl), carboxyl, formyl, alkylcarbonyl, alkylcarbonylalkyl, alkoxycarbonyl, alkylcarbonyloxy, thiocarbonyl, thio, mercapto, mercaptoalkyl, arylsulfonyl, amino, aminoalkyl, dialkylamino, alkylcarbonylamino, alkylaminocarbonyl, alkoxycarbonylamino, alkylamino, arylamino, diarylamino, alkylcarbonyl, or arylamino-substituted aryl; arylalkylamino, arallylaminocarbonyl, or amido. In certain embodiments, substituents on any group include alkyl, halogen, haloalkyl, cyano, nitro, alkoxy, hydroxyl, hydroxylalkyl, carboxyl, formyl, alkylcarbonyl, alkoxycarbonyl, alkylcarbonyloxy, thio, mercapto, mercaptoalkyl, amino, aminoalkyl, dialkylamino, alkylcarbonylamino, alkylaminocarbonyl, or alkylamino.

The term “protecting group” or “protecting moiety” refers to a substituent that is commonly employed to block or protect a particular functionality while reacting other functional groups on the compound, a derivative thereof, or a conjugate thereof, and includes a nitrogen protecting group when attached to a nitrogen atom, or an oxygen protecting group when attached to an oxygen atom; Nitrogen and oxygen protecting groups are well known in the art and include those described in detail in Protecting Groups in Organic Synthesis, T. W. Greene and P. G. M. Wuts, 3rd edition, John Wiley & Sons, 1999, incorporated herein by reference.

In certain embodiments, the substituent present on a nitrogen atom is a nitrogen protecting group (also referred to as an amino protecting group). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein each R^{aa}, R^{bb}, and R^{cc} is independently alkyl, cycloalkyl, aryl, or heteroaryl, each of which may be optionally substituted with 1-3 independent R^{dd}, and each R^{dd} is independently alkyl, alkenyl, alkynyl, cycloalkyl, heterocycloalkyl, aralkyl, heteroaralkyl, aryl, heteroaryl, halogen, haloalkyl, cyano, nitro, alkoxy, aryloxy, hydroxyl, hydroxylalkyl, oxo (i.e., carbonyl), carboxyl, formyl, alkylcarbonyl, alkylcarbonylalkyl, alkoxycarbonyl, alkylcarbonyloxy, aryloxycarbonyl, heteroaryloxy, heteroaryloxycarbonyl, thio, mercapto, mercaptoalkyl, arylsulfonyl, amino, aminoalkyl, dialkylamino, alkylcarbonylamino, alkylaminocarbonyl, alkoxycarbonylamino, alkylamino, arylamino, diarylamino, alkylcarbonyl, or arylamino-substituted aryl; arylalkylamino, aralkylaminocarbonyl, amido, alkylaminosulfonyl, arylaminosulfonyl, dialkylaminosulfonyl, alkylsulfonylamino, arylsulfonylamino, imino, carbamido, carbamyl, thioureido, thiocyanato, sulfoamido, sulfonylalkyl, sulfonylaryl, or mercaptoalkoxy. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Amide nitrogen protecting groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitrophenylacetamide, a nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine, o-nitrobenzamide, and o-(benzoyloxymethyl)benzamide.

Carbamate nitrogen protecting groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluorenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), ally carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonoethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isobornyl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Sulfonamide nitrogen protecting groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to as a hydroxyl protecting group). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenyl methyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, trip-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), t-butyl carbonate (BOC), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a thiol protecting group). Sulfur protecting groups include, but are not limited to, —R^{aa}, —N(R^{bb})₂, —C(═O)SR^{aa}, —C(S)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

“Michael acceptor” means an α,β-unsaturated electrophile, such as, but not limited to, an α,β-unsaturated carbonyl derivative or an α,β-unsaturated nitrile.

It is to be understood that within the context of the definition of Michael acceptor. “Electrophile” means able to accept an electron pair; “α,β-unsaturated electrophile” means the compound class that includes, but is not limited to, α,β-unsaturated carbonyl derivative, α,β-unsaturated nitrile, α,β-unsaturated sulfone, or other vinyl derivative substituted with a strong electron withdrawing group, such as, but not limited to, a nitro group; “α,β-unsaturated carbonyl derivative” means the compound class that includes, but is not limited to, α,β-unsaturated ketone, quinone or derivative thereof, α,β-unsaturated aldehyde, α,β-unsaturated carboxylic acid derivative, such as, but not limited to, an ester, an amide, a substituted amide, or a maleimide or a derivative thereof. Representative examples of Michael acceptors include, but are not limited to, acrylonitrile, acrylamide, methyl acrylate, ethyl acrylate, methyl methacrylate, 2-ethylhexyl acrylate, crotonaldehyde, methyl vinyl ketone, and acrolein.

A “Michael acceptor, or electrophile, that can react with a nucleic acid” includes such species which can interact adversely with a nucleic acid, for example, species which can participate in potentially genotoxic reactions with nucleic acid nucleophiles.

Reaction of Michael acceptors with organic thiols to form thiol-Michael adducts is a well-documented reaction (Chem. Commun. 2005, 669-671 and work cited therein). The reaction has been shown to proceed in water and organic solvents. Both acidic and basic catalysts have been used to facilitate thiol-Michael adduct formation and minimize side reactions.

In one aspect, the compounds described herein are oligomeric compounds. As used herein, an “oligomeric compound” is a nucleotide sequence containing about 10-50 nucleotides or nucleotide base pairs. In some embodiments, an oligomeric compound has a nucleobase sequence that is at least partially complementary to a coding sequence in an expressed target nucleic acid or target gene within a cell. In some embodiments, the oligomeric compounds, upon delivery to a cell expressing a gene, are able to inhibit the expression of the underlying gene. The gene expression can be inhibited in vitro or in vivo. “Oligomeric compounds” include, but are not limited to: oligonucleotides, single-stranded oligonucleotides, single-stranded antisense oligonucleotides, short interfering RNAs (siRNAs), double-stranded RNAs (dsRNA), micro RNAs (miRNAs), short hairpin RNAs (shRNA), ribozymes, and interfering RNA molecules.

As used herein a “nucleic acid” (e.g, polynucleotide, oligonucleotide, polymer of nucleotides), without limitation, may be comprised of ribonucleic acids (e.g., comprised of ribonucleosides), deoxyribonucleic acids (e.g., comprised of deoxyribonucleosides), modified nucleic acids (e.g., comprised of modified nucleobases, sugars, and/or phosphate groups), or a combination thereof. In some embodiments, a nucleic acid comprises a ribonucleic acid (RNA). In some embodiments, a nucleic acid comprises a deoxyribonucleic acid (DNA). In some embodiments, a nucleic acid comprises a modification (e.g., modified nucleobase, modified sugar, or modified phosphate).

Nucleic acids may be single-stranded or double-stranded. In some embodiments, a nucleic acid is single-stranded (e.g., ssRNA, ssDNA, ssRNA/DNA hybrid (e.g., a single-stranded nucleic acid comprised of both ribonucleosides (modified or unmodified) and deoxyribonucleosides (modified or unmodified)). In some embodiments, a nucleic acid is double-stranded (e.g., comprised of two single-stranded nucleic acids).

In some embodiments, a nucleic acid is at least 2 (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or more) nucleotides in length. In some embodiments, a nucleic acid is at least 5 nucleotides in length. In some embodiments, a nucleic acid is at least 10 nucleotides in length. In some embodiments, a nucleic acid is at least 20 nucleotides in length. In some embodiments, a nucleic acid is at least 30 nucleotides in length. In some embodiments, a nucleic acid is at least 40 nucleotides in length. In some embodiments, a nucleic acid is at least 50 nucleotides in length. In some embodiments, a nucleic acid is at least 60 nucleotides in length. In some embodiments, a nucleic acid is at least 70 nucleotides in length. In some embodiments, a nucleic acid is at least 80 nucleotides in length. In some embodiments, a nucleic acid is at least 90 nucleotides in length. In some embodiments, a nucleic acid is at least 100 nucleotides in length. In some embodiments, a nucleic acid is at least 150 nucleotides in length.

In some embodiments, a nucleic acid is less than or equal to 150 (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150) nucleotides in length. In some embodiments, a nucleic acid is less than or equal to 150 nucleotides in length. In some embodiments, a nucleic acid is less than or equal to 100 nucleotides in length. In some embodiments, a nucleic acid is less than or equal to 90 nucleotides in length. In some embodiments, a nucleic acid is less than or equal to 80 nucleotides in length. In some embodiments, a nucleic acid is less than or equal to 70 nucleotides in length. In some embodiments, a nucleic acid is less than or equal to 60 nucleotides in length. In some embodiments, a nucleic acid is less than or equal to 50 nucleotides in length. In some embodiments, a nucleic acid is less than or equal to 40 nucleotides in length. In some embodiments, a nucleic acid is less than or equal to 30 nucleotides in length. In some embodiments, a nucleic acid is less than or equal to 20 nucleotides in length. In some embodiments, a nucleic acid is less than or equal to 10 nucleotides in length. In some embodiments, a nucleic acid is less than or equal to 5 nucleotides in length.

In some embodiments, a nucleic acid is about 5 nucleotides in length to about 150 nucleotides in length. In some embodiments, a nucleic acid is about 10 nucleotides in length to about 100 nucleotides in length. In some embodiments, a nucleic acid is about 20 nucleotides in length to about 90 nucleotides in length. In some embodiments, a nucleic acid is about 30 nucleotides in length to about 80 nucleotides in length. In some embodiments, a nucleic acid is about 40 nucleotides in length to about 70 nucleotides in length. In some embodiments, a nucleic acid is about 50 nucleotides in length to about 60 nucleotides in length.

In some embodiments, a nucleic acid is a therapeutic nucleic acid. A therapeutic nucleic acid may comprise, for example, without limitation, a small interfering RNA (siRNA), a micro RNA (miRNA), an ADAR recruiting molecule, an ADAR targeting molecule, a guide RNA, an antisense nucleic acid, or combinations thereof.

The terms “microRNA” and “miRNA,” as may be used interchangeably herein, refer to short (e.g., about 20 to about 24 nucleotides in length) non-coding ribonucleic acids (RNAs) that are involved in post-transcriptional regulation of gene expression in multicellular organisms by affecting both the stability and translation of mRNAs. miRNAs are transcribed by RNA polymerase II as part of capped and polyadenylated primary transcripts (pri-miRNAs) that can be either protein-coding or non-coding. The primary transcript is cleaved by the Drosha ribonuclease III enzyme to produce a stem-loop precursor miRNA (pre-miRNA) approximately 70 nucleotides in length, which is further processed in the RNAi pathway. As part of this pathway the pre-miRNA is cleaved by the cytoplasmic Dicer ribonuclease to generate the mature miRNA and antisense miRNA star (miRNA*) products. The mature miRNA is incorporated into an RNA-induced silencing complex (RISC), which recognizes target mRNAs through imperfect base pairing (i.e., partial complementarity) with the miRNA and most commonly results in translational inhibition or destabilization of the target mRNA. This mechanism is most often seen through the binding of the miRNA on the 3′ untranslated region (UTR) of the target mRNA, which can decrease gene expression by either inhibiting translation (for example, by blocking the access of ribosomes for translation) or directly causing degradation of the transcript. The term (i.e., miRNA) may be used herein to any form of the subject miRNA (e.g., precursor, primary, and/or mature miRNA).

The terms “small interfering RNA” and “siRNA,” as may be used interchangeably herein, refer to RNA molecules which present as non-coding double-stranded RNA (dsRNA) molecules of about 20 to about 24 nucleotides in length (approximately similar to miRNA) which are useful in RNA interference (RNAi). siRNA are often found with phosphorylated 5′ ends and hydroxylated 3′ ends, which 3′ ends typically have a 2 nucleotide overhang beyond the 5′ end of the anti-parallel strand (e.g., complementary strand of the dsRNA molecule). siRNA are most often found interfering with the expression of specific genes through binding of target sequences (e.g., target gene sequences) to which they are complementary and promoting (e.g., facilitating, triggering, initiating) degradation of the mRNA, thereby preventing (e.g., inhibiting, silencing, interfering with) translation. After integration and separation into the RISC complex, siRNAs base-pair (e.g., full complementary) to their target mRNA and cleave it, thereby preventing it from being used as a translation template. As discussed herein above, also part of the RNAi pathway, an miRNA-loaded RISC complex scans cytoplasmic mRNAs for potential complementarity (e.g., partial complementarity).

The term “ADAR recruiting molecule,” as may be used herein, refers to a nucleic acid that is configured to increase the concentration of Adenosine Deaminase Acting on Ribonucleic Acid (ADAR) enzyme in a locality around the nucleic acid. In some embodiments, an increased concentration is relative to the concentration in a given locality absent the ADAR recruiting molecule. In some embodiments, an ADAR recruiting molecule comprises a double-stranded RNA duplex.

The term “ADAR targeting molecule,” as may be used herein, refers to a nucleic acid which is configured to direct an ADAR molecule to a desirable location (e.g., locality). As used herein, the term “direct” refers to increasing the concentration of ADAR in the desirable location as compared to the concentration absent the ADAR targeting molecule. In some embodiments, the ADAR targeting molecule can be configured to control the desirable location by altering the sequence and/or properties of the nucleic acid (e.g., by modifications to the nucleobase, sugar, phosphate, or other component). In some embodiments, an ADAR targeting molecule comprises an ADAR recruiting molecule and a single-stranded guide nucleic acid. In some embodiments, an ADAR targeting molecule comprises a double-stranded RNA duplex and a single-stranded guide nucleic acid.

The term “single-stranded guide nucleic acid,” as may be used herein, refers to a nucleic acid of a single strand, which comprises a specific sequence that is at least partially complementary to a target sequence. In some embodiments, the target sequence is at, adjacent to, or in proximity to, a locality where it is desirable to modulate ADAR concentration. In some embodiments, the level of complementarity is sufficient to facilitate binding (e.g., annealing) of the single-stranded guide nucleic acid to the target sequence.

The term “antisense molecule,” as may be used herein, refers to an oligonucleotide (e.g., polymer of nucleotides) which is synthesized or contains a sequence of nucleotides complementary to a target nucleic acid sequence. For example, with respect to RNA (e.g., mRNA, miRNA), a strand may read 5′-AAGGUCCU-3′, wherein the antisense molecule will read 3′-UUCCAGGA-5′. In the case of antisense molecules targeting RNA, they can modulate expression in a variety of ways. For example, strands may target mRNA (thereby blocking translation and promoting degradation of the mRNA transcript) or in another manner, the strands may target miRNA (thereby inhibiting the blocking miRNA from targeting the mRNA and promoting or restoring translation from the mRNA, and promoting degradation of the blocking miRNA).

In some embodiments, a nucleic acid is conjugated to a GalNAc moiety. GalNAc (N-Acetylgalactosamine) is an amino sugar derivative of galactose. In some embodiments, a GalNAc moiety comprises the structure

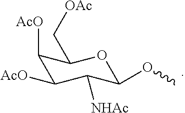

In some embodiments, a GalNAc moiety comprises the structure

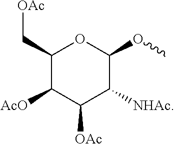

GalNAc moieties are moieties that have an affinity for various tissues and cell receptors. In this way, GalNAc moieties can facilitate the targeting of cargo (e.g., nucleic acids) to such tissues and receptors. In some embodiments, a GalNAc moiety is useful for directing nucleic acids. In some embodiments, a GalNAc moiety directs a nucleic acid to a locality. In some embodiments, a GalNAc moiety targets tissues. In some embodiments, the tissue is liver. In some embodiments, a GalNAc moiety targets a cell receptor. In some embodiments, a cell receptor is an asialoglycoprotein receptor. In some embodiments, an asialoglycoprotein receptor on a hepatocyte.

In some embodiments, a nucleic acid is conjugated to more than one GalNAc moiety. In some embodiments, a nucleic acid is conjugated to at least two GalNAc moieties (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or more). In some embodiments, a nucleic acid is conjugated to at least three GalNAc moieties. In some embodiments, a nucleic acid is conjugated to at least five GalNAc moieties. In some embodiments, a nucleic acid is conjugated to at least 1 to about 10 GalNAc moieties. In some embodiments, a nucleic acid is conjugated to at least 1 to about 8 GalNAc moieties. In some embodiments, a nucleic acid is conjugated to at least 1 to about 6 GalNAc moieties. In some embodiments, a nucleic acid is conjugated to at least 1 to about 4 GalNAc moieties. In some embodiments, a nucleic acid is conjugated to at least 1 to about 2 GalNAc moieties. In some embodiments, a nucleic acid is conjugated to at least 1 to 10 GalNAc moieties. In some embodiments, a nucleic acid is conjugated to at least 1 to 8 GalNAc moieties. In some embodiments, a nucleic acid is conjugated to at least 1 to 6 GalNAc moieties. In some embodiments, a nucleic acid is conjugated to at least 1 to 4 GalNAc moieties. In some embodiments, a nucleic acid is conjugated to at least 1 to 2 GalNAc moieties.

In some embodiments, a nucleic acid is conjugated (e.g., connected, attached, associated by) to a GalNAc moiety through either a 5′ end and/or a 3′ end of the nucleic acid. In some embodiments, a nucleic acid is conjugated to a GalNAc moiety through the 5′ end of the nucleic acid. In some embodiments, a nucleic acid is conjugated to a GalNAc moiety through the 3′ end of the nucleic acid. In some embodiments, a nucleic acid is conjugated to a GalNAc moiety through both the 5′ end and the 3′ end of the nucleic acid. In some embodiments, a nucleic acid is conjugated to a GalNAc moiety at an internal position within the nucleic acid.

In some aspects, the disclosure relates to methods of making the compositions comprising nucleic acids and GalNAc as disclosed herein.

In another aspect, the invention provides conjugates of the compound formulae herein. In one embodiment, the conjugates comprise a moiety of the formulae of the invention covalently coupled to a moiety that is a protein, nucleic acid, small molecule, large molecule, therapeutic, diagnostic, imaging, or targeting agent.

Conjugates as disclosed herein can be manufactured using any available method. When associating compound formulae moieties with agent moieties (e.g., protein, nucleic acid, small molecule, large molecule, therapeutic, diagnostic, imaging, or targeting agent), the moieties may be linked directly or indirectly (e.g., through a linker moiety; that is, the linker is covalently bonded to each of the compound formulae moiety and the agent moiety; in some formulae herein “-L-”). For example, the compound formulae and agent may be directly associated with one another, e.g., by one or more covalent bonds, or may be associated by means of one or more linkers.

The “therapeutic” can be a therapeutic agent for modulating hepatocytes or treating liver disease, such as viral hepatitis, liver fibrosis, non-alcoholic steatohepatitis (NASH), non-alcoholic fatty liver disease (NAFLD), rare disease, metabolic disease, obesity, cardiovascular disease hemochromatosis, thalassemia, liver injury, alcoholic liver disease, or liver genetic disease, including oligonucleotides, small molecules, or peptides, including but not limited to for example, ursodiol, methylclothiazide, pioglitazone, metadoxine, cystadane, ondansetron, silymarin, lamivudine, adefovir, tenofovir disoproxil, tenofovir alafenamide, telbivudine, entecavir, and cholic acid.

Any suitable linker can be used in accordance with the present invention. Linkers may be used to form amide linkages, ester linkages, disulfide linkages, etc. Linkers may contain carbon atoms or heteroatoms (e.g., nitrogen, oxygen, sulfur, etc.). Typically, linkers are 1 to 50 atoms long, 1 to 40 atoms long, 1 to 25 atoms long, 1 to 20 atoms long, 1 to 15 atoms long, 1 to 10 atoms long, or 1 to 5 atoms long. Linkers may be substituted with various substituents including, but not limited to, hydrogen atoms, alkyl, alkenyl, alkynyl, amino, alkylamino, dialkylamino, trialkylamino, hydroxyl, alkoxy, halogen, aryl, heterocyclic, aromatic heterocyclic, cyano, amide, carbamoyl, carboxylic acid, ester, thioether, alkylthioether, thiol, and ureido groups. As would be appreciated by one of skill in this art, each of these groups may in turn be substituted. A linker can an aliphatic or heteroaliphatic linker. For example, the linker can a polyalkyl linker. The linker can be a polyether linker. The linker can be a polyethylene linker, such as PEG. The linker can be a short peptide chain, e.g., between 1 and 10 amino acids in length, e.g., 1, 2, 3, 4, or 5 amino acids in length, a nucleic acid, an alkyl chain, etc.

Compounds and oligonucleotides of the present disclosure can be made by means known in the art of organic synthesis. Methods for optimizing reaction conditions, and minimizing competing by-products if necessary, are known in the art. Reaction optimization and scale-up may advantageously utilize high-speed parallel synthesis equipment and computer-controlled microreactors (e.g. Design And Optimization in Organic Synthesis, 2^{nd} Edition, Carlson R, Ed, 2005; Elsevier Science Ltd.; Jähnisch, K et al, Angew. Chem. Int. Ed. Engl. 2004 43: 406; and references therein). Additional reaction schemes and protocols may be determined by the skilled artisan by use of commercially available structure-searchable database software, for instance, SciFinder® (CAS division of the American Chemical Society) and CrossFire Beilstein® (Elsevier MDL), or by appropriate keyword searching using an Internet search engine such as Google® or keyword databases such as the US Patent and Trademark Office text database.

As can be appreciated by the skilled artisan, methods of synthesizing the compounds and oligonucleotides of the formulae herein will be evident to those of ordinary skill in the art, including in the schemes and examples herein. Additionally, the various synthetic steps may be performed in an alternate sequence or order to give the desired compounds and oligonucleotides. In addition, the solvents, temperatures, reaction durations, etc. delineated herein are for purposes of illustration only and one of ordinary skill in the art will recognize that variation of the reaction conditions can produce the desired compounds and oligonucleotides of the present disclosure.

The compounds and oligonucleotides herein may also contain linkages (e.g., carbon-carbon bonds) wherein bond rotation is restricted about that particular linkage, e.g. restriction resulting from the presence of a ring or double bond. Accordingly, all cis/trans and E/Z isomers are expressly included in the present disclosure. The compounds and oligonucleotides herein may also be represented in multiple tautomeric forms, in such instances, the present disclosure expressly includes all tautomeric forms of the compounds and oligonucleotides described herein, even though only a single tautomeric form may be represented. All such isomeric forms of such compounds and oligonucleotides herein are expressly included in the present disclosure. All crystal forms and polymorphs of the compounds and oligonucleotides described herein are expressly included in the present disclosure. Also embodied are extracts and fractions comprising compounds and oligonucleotides of the present disclosure. The term “isomers” is intended to include diastereoisomers, enantiomers, regioisomers, structural isomers, rotational isomers, tautomers, and the like. For compounds and oligonucleotides which contain one or more stereogenic centers, e.g., chiral compounds, the methods of the present disclosure may be carried out with an enantiomerically enriched compound, a racemate, or a mixture of diastereomers. All isomers of compounds delineated herein are expressly included in the present disclosure.

Preferred enantiomerically enriched compounds have an enantiomeric excess of 50% or more, more preferably the compound has an enantiomeric excess of 60%, 70%, 80%, 90%, 95%, 98%, or 99% or more. In preferred embodiments, only one enantiomer or diastereomer of a chiral compound of the present disclosure is administered to cells or a subject.

### Methods of Treatment

In one aspect, provided are methods of treating a subject suffering from or susceptible to a disorder or disease, comprising administering to the subject an effective amount of a compound, oligonucleotide, or pharmaceutical composition of any of the formula herein (e.g., Formula I-XXXIV).

In other aspects, provided are methods of treating a subject suffering from or susceptible to a disorder or disease, wherein the subject has been identified as in need of modulation of the function of a protein, comprising administering to said subject in need thereof, an effective amount of a compound, oligonucleotide, or pharmaceutical composition of any of the formula herein (e.g., Formula I-XXXIV), such that said subject is treated for said disorder.

In one aspect, provided are methods of modulating protein function in a subject, comprising contacting the subject with a compound or oligonucleotide of any of the formula herein (e.g., Formula I-XXXIV), in an amount and under conditions sufficient to modulate protein function.

In one embodiment, the modulation is inhibition.

In some embodiments, provided are methods for targeting hepatic cells in a subject, comprising administering to said subject in need thereof, an effective amount of a compound, oligonucleotide, or pharmaceutical composition of any of the formula herein (e.g., Formula I-XXXIV) in an amount and under conditions sufficient to target hepatic cells.

In certain embodiments, provided are methods of treating a disease, disorder or symptom thereof, wherein the disorder is cancer, a proliferative disease, a neurodegenerative disease, an autoimmune or inflammatory disorder, an infection, a metabolic disorder, a hematologic disorder, or a cardiovascular disease.

In certain embodiments, the disorder or disease is cancer or a proliferative disease. In certain embodiments, the cancer or proliferative disease includes a carcinoma, a leukemia, a blastoma, a lymphoma, a myeloma, or a melanoma, or a combination thereof. In certain embodiments, the disorder or disease is multiple myeloma, melanoma, breast cancer, pancreatic cancer, ovarian cancer, prostate cancer, hepatocellular cancer, renal cancer, leukemia, T-cell lymphoma, bone cancer, glioblastoma, neuroblastoma, oral squamous cell carcinoma, urothelial cancer, lung cancer, cervical cancer, colon cancer, head and neck squamous cell carcinoma, Burkitt's Lymphoma, esophageal cancer, Hodgkin's lymphoma, bladder cancer, or gastric cancer, or a combination thereof.

In certain embodiments, the disorder or disease is rheumatoid arthritis, spondylitis arthritis, psoriatic arthritis, multiple sclerosis, systemic lupus erythematosus, inflammatory bowel disease, graft versus host disease, transplant rejection, fibrotic disease, Crohn's Disease, type-1 diabetes, eczema, psoriasis, sepsis, airway hyperresponsiveness, ulcerative colitis, or a combination thereof.

In certain embodiments, wherein the disorder or disease is epilepsy, attention deficit disorder, Alzheimer's disease, Parkinson's Disease, Huntington's Disease, amyotrophic lateral sclerosis, spinal muscular atrophy, essential tremor, central nervous system trauma, multiple sclerosis, Charcot-Marie-Tooth (MCT), peripheral neuropathy, or cerebral ischemia, or a combination thereof.

In certain embodiments, the disorder or disease is an infection caused by virus, fungus, or bacteria, or a combination thereof.

In certain embodiments, the disorder or disease is metabolic syndrome, diabetes, obesity, high blood pressure, heart failure, cyst growth in autosomal dominant polycystic kidney disease (ADPKD), or a combination thereof.

In certain embodiments, the disorder or disease is cardiovascular stress, pressure overload, chronic ischemia, infarction-reperfusion injury, hypertension, atherosclerosis, peripheral artery disease, heart failure, hypertrophy, angina, arrhythmias, hypercholesterolemia, atherosclerosis, or stroke, or a combination thereof.

In certain embodiments, the disorder or disease is liver disease.

In certain embodiments, the subject is a mammal, preferably a primate or a human.

In another embodiment, provided are methods as described above, wherein the effective amount of the compound or oligonucleotide of any of the formula herein (e.g., Formula I-XXXIV) is as described above.

In another embodiment, provided are methods as described above, wherein the compound or oligonucleotide of any of the formula herein (e.g., Formula I-XXXIV) is administered intravenously, intramuscularly, subcutaneously, intracerebroventricularly, orally, or topically.

In other embodiments, provided are methods as described above, wherein the compound or oligonucleotide of any of the formula herein (e.g., Formula I-XXXIV) is administered alone or in combination with one or more other therapeutics. In a further embodiment, the additional therapeutic agent is an anti-cancer agent, antifungal agent, cardiovascular agent, anti-inflammatory agent, chemotherapeutic agent, an anti-angiogenesis agent, cytotoxic agent, an anti-proliferation agent, metabolic disease agent, ophthalmologic disease agent, central nervous system (CNS) disease agent, urologic disease agent, or gastrointestinal disease agent.

Another object of the present disclosure is the use of a compound or oligonucleotide as described herein (e.g., a compound or oligonucleotide of Formula I-XXXIV) in the manufacture of a medicament for use in the treatment of a disorder or disease. Another object of the present disclosure is the use of a compound or oligonucleotide as described herein (e.g., a compound or oligonucleotide of Formula I-XXXIV) for use in the treatment of a disorder or disease. Another object of the present disclosure is the use of a compound or oligonucleotide as described herein (e.g., a compound or oligonucleotide of Formula I-XXXIV) in the manufacture of an agricultural composition for use in the treatment or prevention of a disorder or disease in agricultural or agrarian settings.

### Pharmaceutical Compositions

In one aspect, provided are pharmaceutical compositions comprising the compound or oligonucleotide of any of the formula herein (e.g., Formula I-XXXIV) and a pharmaceutically acceptable carrier.

A compound, oligonucleotide, or composition, as described herein, can be administered in combination with one or more additional therapeutic agents (e.g., therapeutically and/or prophylactically active agents). The compounds, oligonucleotides, or compositions can be administered in combination with additional therapeutic agents that improve their activity (e.g., activity (e.g., potency and/or efficacy) in treating a disease in a subject in need thereof, in preventing a disease in a subject in need thereof, and/or in reducing the risk to develop a disease in a subject in need thereof), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound or oligonucleotide described herein and an additional therapeutic agent to exhibit a synergistic effect that is absent in a pharmaceutical composition including one of the compound or oligonucleotide and the additional therapeutic agent, but not both.

The compound, oligonucleotide, or composition can be administered concurrently with, prior to, or subsequent to one or more additional therapeutic agents, which may be useful as, e.g., combination therapies. Therapeutic agents include therapeutically active agents. Therapeutic agents also include prophylactically active agents. Therapeutic agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional therapeutic agent is a therapeutic agent useful for treating and/or preventing a disease (e.g., cancer, proliferative disease, neurodegenerative disease, autoimmune or inflammatory disorder, infection, metabolic disorder, hematologic disorder, cardiovascular disease). Each additional therapeutic agent may be administered at a dose and/or on a time schedule determined for that therapeutic agent. The additional therapeutic agents may also be administered together with each other and/or with the compound or composition described herein in a single dose or administered separately in different doses. The particular combination to employ in a regimen will take into account compatibility of the compound described herein with the additional therapeutic agent(s) and/or the desired therapeutic and/or prophylactic effect to be achieved. In general, it is expected that the additional therapeutic agent(s) in combination be utilized at levels that do not exceed the levels at which they are utilized individually. In some embodiments, the levels utilized in combination will be lower than those utilized individually.

The additional therapeutic agents include, but are not limited to, anti-proliferative agents, anti-cancer agents, anti-angiogenesis agents, anti-inflammatory agents, and immunosuppressants. In certain embodiments, the additional therapeutic agent is an immunotherapy. In certain embodiments, the additional therapeutic agent is an anti-proliferative agent. In certain embodiments, the additional therapeutic agent is an anti-cancer agent. In certain embodiments, the anti-cancer agents include, but are not limited to, epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), cell signaling pathway inhibitors (e.g., tyrosine protein kinase inhibitors), modulators of protein stability (e.g., proteasome inhibitors), Hsp90 inhibitors, glucocorticoids, all-trans retinoic acids, anti-estrogens (e.g., tamoxifen, raloxifene, and megestrol), LHRH agonists (e.g., goserelin and leuprolide), anti-androgens (e.g. flutamide and bicalutamide), photodynamic therapies (e.g., verteporfin (BPD-MA), phthalocyanine, photosensitizer Pc4, and demethoxy-hypocrellin A (2BA-2-DMHA)), nitrogen mustards (e.g., cyclophosphamide, ifosfamide, trofosfamide, chlorambucil, estramustine, and melphalan), nitrosoureas (e.g., carmustine (BCNU) and lomustine (CCNU)), alkylsulphonates (e.g., busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g., methotrexate, dichloromethotrexate, trimetrexate, edatrexate), IMP dehydrogenase inhibitors (e.g., mycophenolic acid, tiazofurin, ribavirin, and EICAR), ribonucleotide reductase inhibitors (e.g., hydroxyurea and deferoxamine), uracil analogs (e.g., 5-fluorouracil (5-FU), floxuridine, doxifluridine, ratitrexed, tegafur-uracil, capecitabine), cytosine analogs (e.g., cytarabine (ara C), cytosine arabinoside, and fludarabine), purine analogs (e.g. mercaptopurine and Thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g., bleomycin A2, bleomycin B2, peplomycin), anthracycline (e.g., daunorubicin, doxorubicin, pegylated liposomal doxorubicin, idarubicin, epirubicin, pirarubicin, zorubicin, mitoxantrone), MDR inhibitors (e.g. verapamil), Ca2+ ATPase inhibitors (e.g., thapsigargin), thalidomide, lenalidomide, pomalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE), ixazomib (NINLARO)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genentech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, camptothecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine.

In certain embodiments, the additional therapeutic agent is an immunotherapy. In certain embodiments, the immunotherapy is useful in the treatment of a cancer. Exemplary immunotherapies include, but are not limited to, T-cell therapies, interferons, cytokines (e.g., tumor necrosis factor, interferon α, interferon γ), vaccines, hematopoietic growth factors, monoclonal serotherapy, immunostimulants and/or immunodulatory agents (e.g., IL-1, 2, 4, 6, or 12), immune cell growth factors (e.g., GM-CSF) and antibodies. In certain embodiments, the immunotherapy is a T-cell therapy. In certain embodiments, the T-cell therapy is chimeric antigen receptor T cells (CAR-T). In certain embodiments, the immunotherapy is an antibody. In certain embodiments, the antibody is an anti-PD-1 antibody, an anti-PD-L1 antibody, an anti-CTLA-4 antibody, an anti-TIM3 antibody, an anti-OX40 antibody, an anti-GITR antibody, an anti-LAG-3 antibody, an anti-CD137 antibody, an anti-CD27 antibody, an anti-CD28 antibody, an anti-CD28H antibody, an anti-CD30 antibody, an anti-CD39 antibody, an anti-CD40 antibody, an anti-CD47 antibody, an anti-CD48 antibody, an anti-CD70 antibody, an anti-CD73 antibody, an anti-CD96 antibody, an anti-CD160 antibody, an anti-CD200 antibody, an anti-CD244 antibody, an anti-ICOS antibody, an anti-TNFRSF25 antibody, an anti-TMIGD2 antibody, an anti-DNAM1 antibody, an anti-BTLA antibody, an anti-LIGHT antibody, an anti-TIGIT antibody, an anti-VISTA antibody, an anti-HVEM antibody, an anti-Siglec antibody, an anti-GAL1 antibody, an anti-GAL3 antibody, an anti-GAL9 antibody, an anti-BTNL2 (butrophylins) antibody, an anti-B7-H3 antibody, an anti-B7-H4 antibody, an anti-B7-H5 antibody, an anti-B7-H6 antibody, an anti-KIR antibody, an anti-LIR antibody, an anti-ILT antibody, an anti-MICA antibody, an anti-MICB antibody, an anti-NKG2D antibody, an anti-NKG2A antibody, an anti-TGFβ antibody, an anti-TGFβR antibody, an anti-CXCR4 antibody, an anti-CXCL12 antibody, an anti-CCL2 antibody, an anti-IL-10 antibody, an anti-IL-13 antibody, an anti-IL-23 antibody, an anti-phosphatidylserine antibody, an anti-neuropilin antibody, an anti-GalCer antibody, an anti-HER2 antibody, an anti-VEGFA antibody, an anti-VEGFR antibody, an anti-EGFR antibody, or an anti-Tie2 antibody. In certain embodiments, the antibody is pembrolizumab, nivolumab, pidilizumab, ipilimumab, tremelimumab, durvalumab, atezolizumab, avelumab, PF-06801591, utomilumab, PDR001, PBF-509, MGB453, LAG525, AMP-224, INCSHR1210, INCAGN1876, INCAGN1949, samalizumab, PF-05082566, urelumab, lirilumab, lulizumab, BMS-936559, BMS-936561, BMS-986004, BMS-986012, BMS-986016, BMS-986178, IMP321, IPH2101, IPH2201, varilumab, ulocuplumab, monalizumab, MEDI0562, MEDI0680, MEDI1873, MEDI6383, MEDI6469, MEDI9447, AMG228, AMG820, CC-90002, CDX-1127, CGEN15001T, CGEN15022, CGEN15029, CGEN15049, CGEN15027, CGEN15052, CGEN15092, CX-072, CX-2009, CP-870893, lucatumumab, dacetuzumab, Chi Lob 7/4, RG6058, RG7686, RG7876, RG7888, TRX518, MK-4166, MGA271, IMC-CS4, emactuzumab, trastuzumab, pertuzumab, obinutuzumab, cabiralizumab, margetuximab, enoblituzumab, mogamulizumab, panitumumab, carlumab, bevacizumab, rituximab, or cetuximab.

In certain embodiments, the compounds, oligonucleotides, or pharmaceutical compositions described herein can be administered in combination with an anti-cancer therapy including, but not limited to, surgery, radiation therapy, and transplantation (e.g., stem cell transplantation, bone marrow transplantation).

In certain embodiments, the additional therapeutic agent is selected from the group consisting of Akt inhibitors, alkylating agents, androgen receptor antagonists, anti-estrogens, Bcl-2 inhibitors, BRAF kinase inhibitors, BTK inhibitors, CAR-T Cells, anti-CD38 antibodies, CDK inhibitors, anti-CTLA-4 antibodies, ERK/MAPK inhibitors, farnesyltransferase inhibitors, IL-6 inhibitors, immunomodulatory agents, immuno-oncology agents, JAK2/FLT3 inhibitors, kinesin spindle protein inhibitors, MEK inhibitors, anti-PD-1 antibodies, anti-PD-L1 antibodies, PI3K inhibitors, proteasome inhibitors, radiation (sensitizer), radioisotopes (sensitizer), synthetic retinoids (AM80), taxanes, tyrosine kinase inhibitors, VDR agonists, VEGF inhibitors, oncolytic viruses, and a combination thereof. In certain embodiments, the additional therapeutic agent is selected from the group consisting of all trans tetinoic acid (ATRA), arsenic trioxide, berberine, bevacizumab, bortezomib, cabazitaxel, carfilzomib, cisplatin, clarithromycin, cyclophosphamide, cytarabine, darzalex, dexamethasone, docetaxel, elotuzumab, enzalutamide, epirubicin, fluorouracil (5-FU), gefitinib, gemcitabine hydrochloride, ibiutinib, idelalisib, indatuximab, ixazomib, ravtansine, ipilimumab, lenalidomide, lonafarnib, methotrexate, nab-paclitaxel, nivolumab, paclitaxel, pacritinib, pomalidomide, sorafenib, temozolomide, thalidomide, vemurafenib, and vincristine.

In one aspect, provided are kits comprising an effective amount of a compound or oligonucleotide of any of the formulae herein (e.g., Formula I-XXXIV), in unit dosage form, together with instructions for administering the compound or oligonucleotide to a subject suffering from or susceptible to a disease or disorder, including cancer, proliferative disease, neurodegenerative disease, autoimmune or inflammatory disorder, infection, metabolic disorder, hematologic disorder, and cardiovascular disease. In other embodiments the disease, disorder or symptom thereof is a carcinoma, a leukemia, a blastoma, a lymphoma, a myeloma, or a melanoma. In other embodiments the disease, disorder or symptom thereof is multiple myeloma, melanoma, breast cancer, pancreatic cancer, ovarian cancer, prostate cancer, hepatocellular cancer, renal cancer, leukemia, T cell lymphoma, bone cancer, glioblastoma, neuroblastoma, oral squamous cell carcinoma, urothelial cancer, lung cancer, cervical cancer, colon cancer, head and neck squamous cell carcinoma, Burkitt's Lymphoma, esophageal cancer, Hodgkin's lymphoma, bladder cancer, or gastric cancer. In other embodiments the disease, disorder or symptom thereof is rheumatoid arthritis, spondylitis arthritis, psoriatic arthritis, multiple sclerosis, systemic lupus erythematosus, inflammatory bowel disease, graft versus host disease, transplant rejection, fibrotic disease, Crohn's Disease, type-1 diabetes, eczema, psoriasis, sepsis, airway hyperresponsiveness, or ulcerative colitis. In other embodiments the disease, disorder or symptom thereof is epilepsy, attention deficit disorder, Alzheimer's disease, Parkinson's Disease, Huntington's Disease, amyotrophic lateral sclerosis, spinal muscular atrophy, essential tremor, central nervous system trauma, multiple sclerosis, Charcot-Marie-Tooth (MCT), peripheral neuropathy, or cerebral ischemia. In other embodiments the disease, disorder or symptom thereof is an infection caused by vino, fungus, or bacteria. In other embodiments the disease, disorder or symptom thereof is metabolic syndrome, diabetes, obesity, high blood pressure, heart failure, or cyst growth in autosomal dominant polycystic kidney disease (ADPKD). In other embodiments the disease, disorder or symptom thereof is cardiovascular stress, pressure overload, chronic ischemia, infarction-reperfusion injury, hypertension, atherosclerosis, peripheral artery disease, heart failure, hypertrophy, angina, arrhythmias, hypercholesterolemia, atherosclerosis, or stroke.

The term “pharmaceutically acceptable salts” or “pharmaceutically acceptable carrier” is meant to include salts of the active compounds which are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydroiodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present disclosure contain both basic and acidic functionalities that allow the compounds to be converted into either base or acid addition salts. Other pharmaceutically acceptable carriers known to those of skill in the art are suitable for the present disclosure.

The neutral forms of the compounds and oligonucleotides may be regenerated by contacting the salt with a base or acid and isolating the parent compound or oligonucleotide in the conventional manner. The parent form of the compound or oligonucleotide differs from the various salt forms in certain physical properties, such as solubility in polar solvents, but otherwise the salts are equivalent to the parent form of the compound for the purposes of the present disclosure.

In addition to salt forms, the present disclosure provides compounds which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

Certain compounds of the present disclosure can exist in unsolvated forms as well as solvated forms, including hydrated forms. In general, the solvated forms are equivalent to unsolvated forms and are intended to be encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

The present disclosure also provides a pharmaceutical composition, comprising an effective amount a compound described herein and a pharmaceutically acceptable carrier. In an embodiment, a compound or oligonucleotide of any of the formula herein (e.g., Formula I-XXXIV) is administered to a subject using a pharmaceutically-acceptable formulation, e.g., a pharmaceutically-acceptable formulation that provides sustained delivery of the compound to a subject for at least 12 hours, 24 hours, 36 hours, 48 hours, one week, two weeks, three weeks, or four weeks after the pharmaceutically-acceptable formulation is administered to the subject.

Actual dosage levels and time course of administration of the active ingredients in the pharmaceutical compositions of the disclosure may be varied so as to obtain an amount of the active ingredient which is effective to achieve the desired therapeutic response for a particular patient, composition, and mode of administration, without being toxic (or unacceptably toxic) to the patient.

In use, at least one compound or oligonucleotide according to the present disclosure is administered in a pharmaceutically effective amount to a subject in need thereof in a pharmaceutical carrier by intravenous, intramuscular, subcutaneous, or intracerebroventricular injection or by oral administration or topical application. In accordance with the present disclosure, a compound or oligonucleotide of the disclosure may be administered alone or in conjunction with a second, different therapeutic. By “in conjunction with” is meant together, substantially simultaneously, or sequentially. In one embodiment, a compound or oligonucleotide of the disclosure is administered acutely. The compound or oligonucleotide of the disclosure may therefore be administered for a short course of treatment, such as for about 1 day to about 1 week. In another embodiment, the compound or oligonucleotide of the disclosure may be administered over a longer period of time to ameliorate chronic disorders, such as, for example, for about one week to several months depending upon the condition to be treated.

By “pharmaceutically effective amount” as used herein is meant an amount of a compound or oligonucleotide of the disclosure, high enough to significantly positively modify the condition to be treated but low enough to avoid serious side effects (at a reasonable benefit/risk ratio), within the scope of sound medical judgment. A pharmaceutically effective amount of a compound or oligonucleotide of the disclosure will vary with the particular goal to be achieved, the age and physical condition of the patient being treated, the severity of the underlying disease, the duration of treatment, the nature of concurrent therapy and the specific compound employed. For example, a therapeutically effective amount of a compound or oligonucleotide of the disclosure administered to a child or a neonate will be reduced proportionately in accordance with sound medical judgment. The effective amount of a compound or oligonucleotide of the disclosure will thus be the minimum amount which will provide the desired effect.

A decided practical advantage of the present disclosure is that the compound or oligonucleotide may be administered in a convenient manner such as by intravenous, intramuscular, subcutaneous, oral, or intra-cerebroventricular injection routes or by topical application, such as in creams or gels. Depending on the route of administration, the active ingredients which comprise a compound or oligonucleotide of the disclosure may be required to be coated in a material to protect the compound or oligonucleotide from the action of enzymes, acids and other natural conditions which may inactivate the compound or oligonucleotide. In order to administer a compound or oligonucleotide of the disclosure by a mode other than parenteral administration, the compound or oligonucleotide can be coated by, or administered with, a material to prevent inactivation.

The compound or oligonucleotide may be administered parenterally or intraperitoneally. Dispersions can also be prepared, for example, in glycerol, liquid polyethylene glycols, and mixtures thereof, and in oils.

Some examples of substances which can serve as pharmaceutical carriers are sugars, such as lactose, glucose and sucrose; starches such as corn starch and potato starch; cellulose and its derivatives such as sodium carboxymethylcellulose, ethylcellulose and cellulose acetates; powdered tragacanth; malt; gelatin; talc; stearic acids; magnesium stearate; calcium sulfate; vegetable oils, such as peanut oils, cotton seed oil, sesame oil, olive oil, corn oil, and oil of theobroma; polyols such as propylene glycol, glycerine, sorbitol, mannitol, and polyethylene glycol; agar; alginic acids; pyrogen-free water; isotonic saline; and phosphate buffer solution; skim milk powder; as well as other non-toxic compatible substances used in pharmaceutical formulations such as Vitamin C, estrogen and echinacea, for example. Wetting agents and lubricants such as sodium lauryl sulfate, as well as coloring agents, flavoring agents, lubricants, excipients, tableting agents, stabilizers, anti-oxidants, and preservatives, can also be present. Solubilizing agents, including for example, cremaphore, and beta-cyclodextrins can also used in the pharmaceutical compositions herein.

Pharmaceutical compositions comprising the active compounds or oligonucleotides of the present disclosure (or prodrugs thereof) can be manufactured by means of conventional mixing, dissolving, granulating, dragee-making levigating, emulsifying, encapsulating, entrapping or lyophilization processes. The compositions can be formulated in conventional manner using one or more physiologically acceptable carriers, diluents, excipients or auxiliaries which facilitate processing of the active compounds into preparations which can be used pharmaceutically.

Pharmaceutical compositions of the present disclosure subject matter can take a form suitable for virtually any mode of administration, including, for example, topical, ocular, oral, buccal, systemic, nasal, injection, transdermal, rectal, vaginal, and the like, or a form suitable for administration by inhalation or insufflation.

For topical administration, the active compound(s) or prodrug(s) can be formulated as solutions, gels, ointments, creams, suspensions, and the like.

Systemic formulations include those designed for administration by injection, e.g., subcutaneous, intravenous, intramuscular, intrathecal, or intraperitoneal injection, as well as those designed for transdermal, transmucosal, oral, or pulmonary administration.

Useful injectable preparations include sterile suspensions, solutions, or emulsions of the active compound(s) or oligonucleotide(s) in aqueous or oily vehicles. The compositions also can contain formulating agents, such as suspending, stabilizing and/or dispersing agent. The formulations for injection can be presented in unit dosage form (e.g., in ampules or in multidose containers) and can contain added preservatives.

Alternatively, the injectable formulation can be provided in powder form for reconstitution with a suitable vehicle, including but not limited to sterile pyrogen free water, buffer, dextrose solution, and the like, before use. To this end, the active compound(s) or oligonucleotide(s) can be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions can take the form of, for example, lozenges, tablets, or capsules prepared by conventional means with pharmaceutically acceptable excipients such as binding agents (e.g., pregelatinized maize starch, polyvinylpyrrolidone, or hydroxypropyl methylcellulose); fillers (e.g., lactose, microcrystalline cellulose or calcium hydrogen phosphate); lubricants (e.g., magnesium stearate, talc, or silica); disintegrants (e.g., potato starch or sodium starch glycolate); or wetting agents (e.g., sodium lauryl sulfate). The tablets can be coated by methods well known in the art with, for example, with sugars or enteric coatings.

Liquid preparations for oral administration can take the form of, for example, elixirs, solutions, syrups, or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives, or hydrogenated edible fats); emulsifying agents (e.g., lecithin or acacia); non aqueous vehicles (e.g., almond oil, oily esters, ethyl alcohol, or fractionated vegetable oils); and preservatives (e.g., methyl or propyl p-hydroxybenzoates or sorbic acid). The preparations also can contain buffer salts, preservatives, flavoring, coloring and sweetening agents as appropriate.

Preparations for oral administration can be suitably formulated to give controlled release of the active compound or prodrug, as is well known.

For buccal administration, the compositions can take the form of tablets or lozenges formulated in a conventional manner.

For rectal and vaginal routes of administration, the active compound(s) or oligonucleotide(s) can be formulated as solutions (for retention enemas), suppositories, or ointments containing conventional suppository bases, such as cocoa butter or other glycerides.

For nasal administration or administration by inhalation or insufflation, the active compound(s), oligonucleotide(s), or prodrug(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide, or other suitable gas. In the case of a pressurized aerosol, the dosage unit can be determined by providing a valve to deliver a metered amount. Capsules and cartridges for use in an inhaler or insufflator (for example capsules and cartridges comprised of gelatin) can be formulated containing a powder mix of the compound or oligonucleotide and a suitable powder base such as lactose or starch.

A specific example of an aqueous suspension formulation suitable for nasal administration using commercially-available nasal spray devices includes the following ingredients: active compound or prodrug (0.5-20 mg/ml); benzalkonium chloride (0.1-0.2 mg/mL); polysorbate 80 (TWEEN® 80; 0.5-5 mg/ml); carboxymethylcellulose sodium or microcrystalline cellulose (1-15 mg/ml); phenylethanol (1-4 mg/ml); and dextrose (20-50 mg/ml). The pH of the final suspension can be adjusted to range from about pH5 to pH7, with a pH of about pH 5.5 being typical.

For ocular administration, the active compound(s), oligonucleotide(s), or prodrug(s) can be formulated as a solution, emulsion, suspension, and the like, suitable for administration to the eye. A variety of vehicles suitable for administering compounds to the eye are known in the art. Specific non-limiting examples are described in U.S. Pat. Nos. 6,261,547; 6,197,934; 6,056,950; 5,800,807; 5,776,445; 5,698,219; 5,521,222; 5,403,841; 5,077,033; 4,882,150; and 4,738,851, each of which is incorporated herein by reference in its entirety.

For prolonged delivery, the active compound(s), oligonucleotide(s), or prodrug(s) can be formulated as a depot preparation for administration by implantation or intramuscular injection. The active ingredient can be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the active compound(s) or oligonucleotide(s) for percutaneous absorption can be used. To this end, permeation enhancers can be used to facilitate transdermal penetration of the active compound(s) or oligonucleotide(s). Suitable transdermal patches are described in for example, U.S. Pat. Nos. 5,407,713; 5,352,456; 5,332,213; 5,336,168; 5,290,561; 5,254,346; 5,164,189; 5,163,899; 5,088,977; 5,087,240; 5,008,110; and 4,921,475, each of which is incorporated herein by reference in its entirety.

Alternatively, other pharmaceutical delivery systems can be employed. Liposomes and emulsions are well-known examples of delivery vehicles that can be used to deliver active compound(s), oligonucleotide(s), or prodrug(s). Certain organic solvents such as dimethylsulfoxide (DMSO) also can be employed.

The pharmaceutical compositions can, if desired, be presented in a pack or dispenser device which can contain one or more unit dosage forms containing the active compound(s) or oligonucleotide(s). The pack can, for example, comprise metal or plastic foil, such as a blister pack. The pack or dispenser device can be accompanied by instructions for administration.

The active compound(s), oligonucleotide(s), or prodrug(s) of the present disclosure, or compositions thereof, will generally be used in an amount effective to achieve the intended result, for example in an amount effective to treat or prevent the particular disease being treated. The compound(s) and oligonucleotide(s) can be administered therapeutically to achieve therapeutic benefit or prophylactically to achieve prophylactic benefit. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient can still be afflicted with the underlying disorder. Therapeutic benefit also includes halting or slowing the progression of the disease, regardless of whether improvement is realized.

For prophylactic administration, the compound or oligonucleotide can be administered to a patient at risk of developing one of the previously described diseases. A patient at risk of developing a disease can be a patient having characteristics placing the patient in a designated group of at risk patients, as defined by an appropriate medical professional or group. A patient at risk may also be a patient that is commonly or routinely in a setting where development of the underlying disease could occur. In other words, the an at risk patient is one who is commonly or routinely exposed to the disease or illness causing conditions or may be acutely exposed for a limited time. Alternatively, prophylactic administration can be applied to avoid the onset of symptoms in a patient diagnosed with the underlying disorder.

The amount of compound administered will depend upon a variety of factors, including, for example, the particular indication being treated, the mode of administration, whether the desired benefit is prophylactic or therapeutic, the severity of the indication being treated and the age and weight of the patient, the bioavailability of the particular active compound, and the like. Determination of an effective dosage is well within the capabilities of those skilled in the art.

Effective dosages can be estimated initially from in vitro assays. For example, an initial dosage for use in animals can be formulated to achieve a circulating blood or serum concentration of active compound that is at or above an IC₅₀ of the particular compound as measured in as in vitro assay, such as the in vitro fungal MIC or MFC and other in vitro assays. Calculating dosages to achieve such circulating blood or serum concentrations taking into account the bioavailability of the particular compound is well within the capabilities of skilled artisans. For guidance, see “General Principles,” In: Goodman and Gilman's The Pharmaceutical Basis of Therapeutics, Chapter 1, pp. 1-112, 13th ed., McGraw-Hill, and the references cited therein, which are incorporated herein by reference.

Initial dosages also can be estimated from in vivo data, such as animal models. Animal models useful for testing the efficacy of compounds to treat or prevent the various diseases described above are well-known in the art.

Dosage amounts will typically be in the range of from about 0.0001 or 0.001 or 0.01 mg/kg/day to about 100 mg/kg/day, but can be higher or lower, depending upon, among other factors, the activity of the compound or oligonucleotide, its bioavailability, the mode of administration, and various factors discussed above. Dosage amount and interval can be adjusted individually to provide plasma levels of the compound(s) or oligonucleotide(s) which are sufficient to maintain therapeutic or prophylactic effect. In cases of local administration or selective uptake, such as local topical administration, the effective local concentration of active compound(s) or oligonucleotide(s) cannot be related to plasma concentration. Skilled artisans will be able to optimize effective local dosages without undue experimentation.

The compound(s) and oligonucleotide(s) can be administered once per day, a few or several times per day, or even multiple times per day, depending upon, among other things, the indication being treated and the judgment of the prescribing physician.

Preferably, the compound(s) and oligonucleotide(s) will provide therapeutic or prophylactic benefit without causing substantial toxicity. Toxicity of the compound(s) and oligonucleotide(s) can be determined using standard pharmaceutical procedures. The dose ratio between toxic and therapeutic (or prophylactic) effect is the therapeutic index. Compounds(s) and oligonucleotide(s) that exhibit high therapeutic indices are preferred.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

### General Experimental Procedures

Definitions of variables in the structures in schemes herein are commensurate with those of corresponding positions in the formulae delineated herein.

### Common Abbreviations

ACN acetonitrile
br broad
d doublet
DCM dichloromethane
dd doublet of doublets
dba dibenzylideneacetone
DFAA difluoroacetic anhydride
DIPEA diisopropylethylamine
DMF dimethylformamide
DMSO dimethyl sulfoxide
dppf 1,1′-ferrocenediyl-bis(diphenylphosphine)
EtOAc ethyl acetate
h hour(s)
HRMS high resolution mass spectrometry
HPLC high performance liquid chromatography
LCMS liquid chromatography and mass spectrometry
MS mass spectrometry
MW microwave
m multiplet
MeOH methanol
min minutes
mL milliliter(s)
m/z mass to charge ratio
NMP N-methyl-2-pyrrolidone
NMR nuclear magnetic resonance
ppm parts per million
rt or RT room temperature
s singlet
t triplet
TFAA trifluoroacetic anhydride
TLC thin layer chromatography

### Example 1

Exemplary compounds falling within the scope of the present disclosure could be synthesized according to the following scheme:

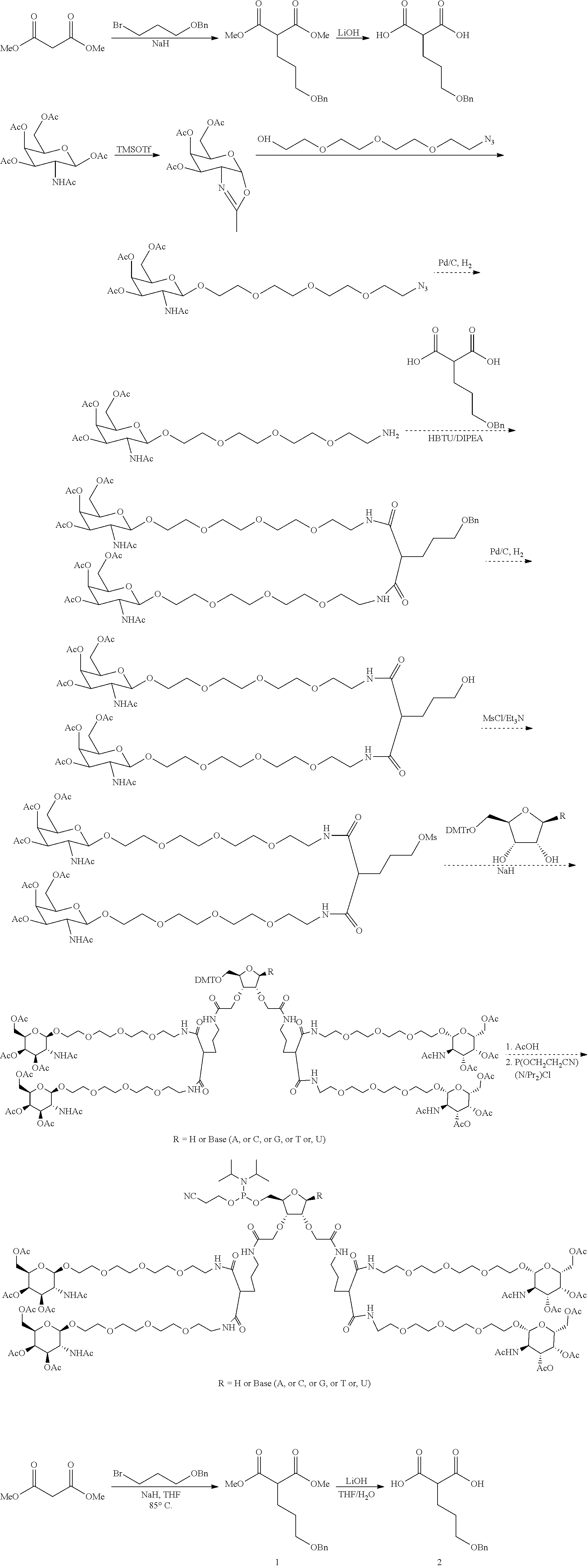

### Dimethyl 2-(3-phenoxypropyl)malonate (1)

To a stirring solution of NaH (1.91 g, 47.8 mmol, 1.2 eq) in THF (50 mL) at room temperature was carefully added dimethyl malonate dropwise (10 g, 75.68 mmol, 1.9 eq). Upon addition, the reaction mixture became a thick suspension. THF (10 mL) was added to the reaction, and it was stirred for 1 h. Benzyl 3-bromopropyl ether (9.126 g, 39.8 mmol, 1 eq) was added to the reaction, and the reaction mixture was heated to 85° C. After stirring for 5 hours, the reaction was cooled down to RT, diluted with EtOAc (100 ml) and washed with water (100 mL). The aqueous phase was extracted with EtOAc (100 mL). The combined organic layers were dried over Na₂SO₄, filtered, and concentrated. The residue was purified by flash chromatography using 0-50% EtOAc in Hexanes to give 10.5 g 1 (94%) as a clear oil. Product was confirmed by LCMS and NMR. HPLC: rt=4.996 min.

### 2-(3 phenoxypropyl)malonic Acid (2)

To a stirring solution of compound 1 (10.5 g, 37.5 mmol, 1 eq) in THF/H₂O (1:1, 100 mL) was added LiOH. The reaction was stirred overnight and then acidified with 1 N HCl to pH=2. The mixture was extracted with EtOAc (100 mL×2), dried over Na₂SO₄, and concentrated to give 8.9 g crude product 2 (93%) as a white solid which was used in the next step without purification. HPLC: rt=3.592 min.

### Example 2

Exemplary compounds falling within the scope of the present disclosure could be synthesized according to the following scheme:

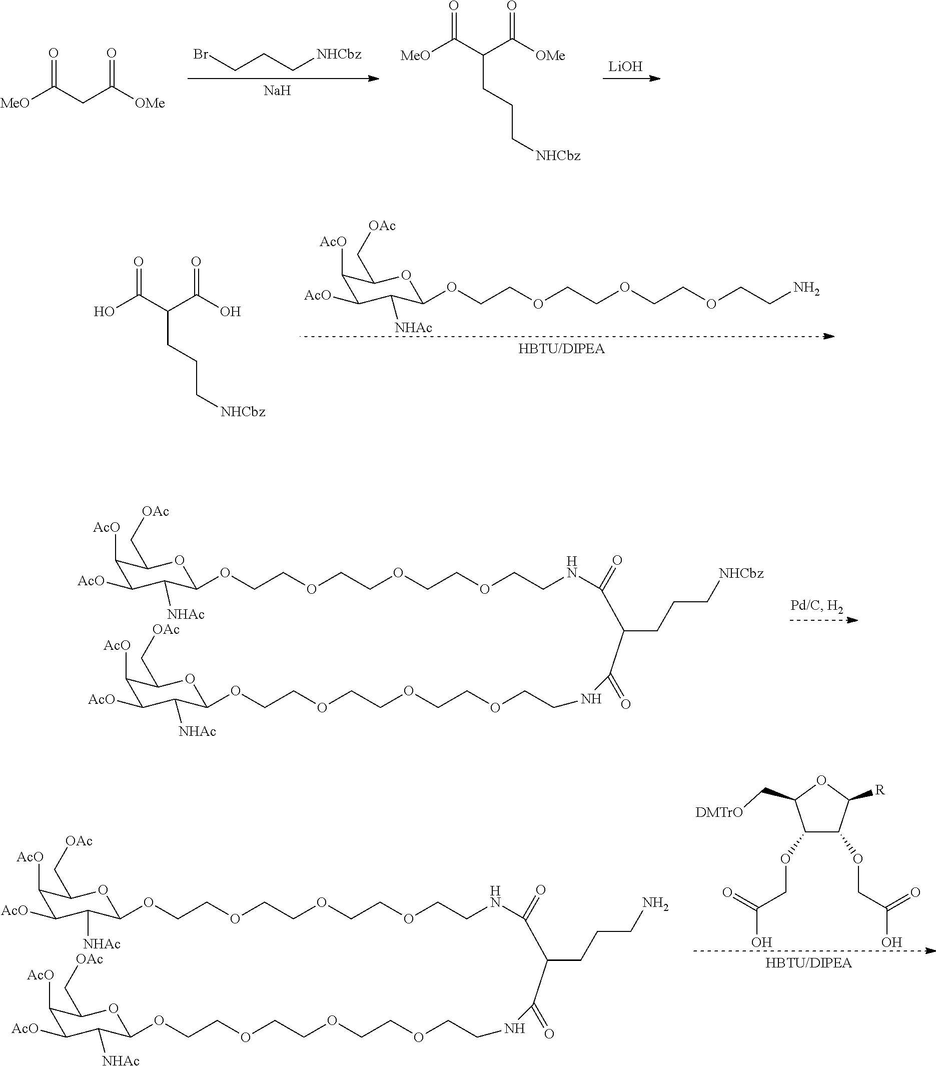

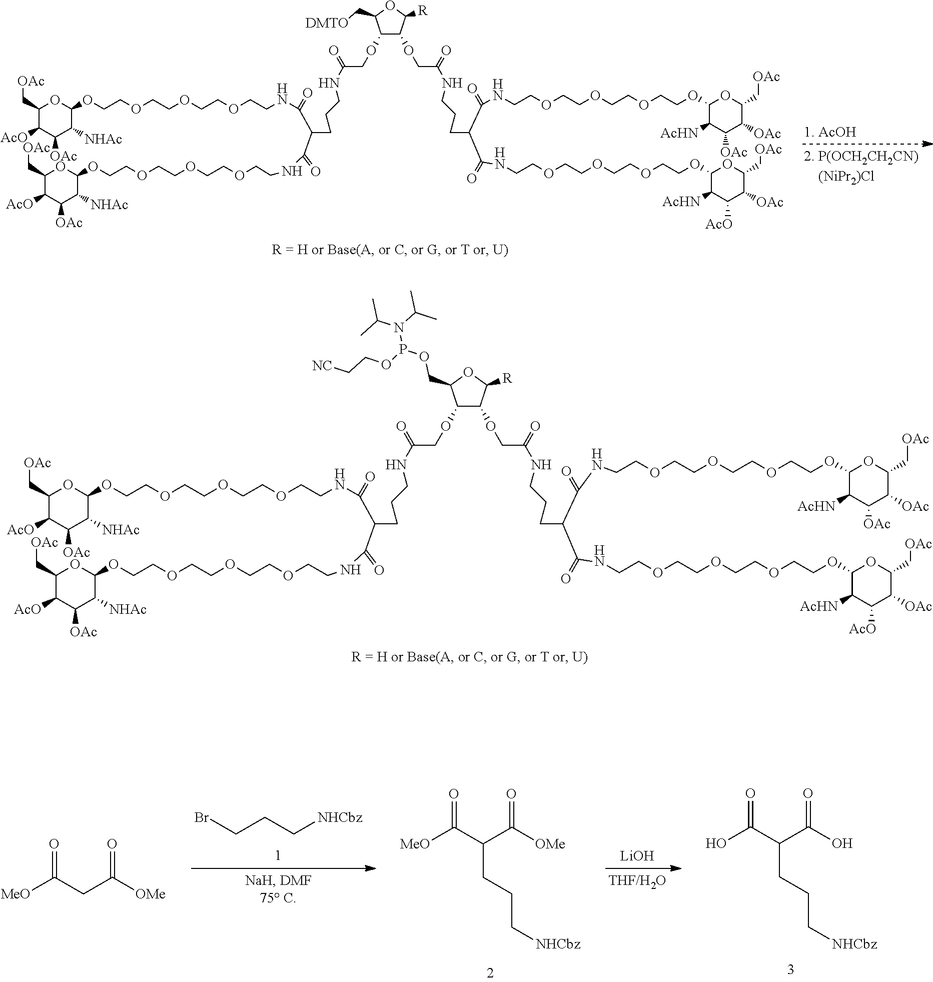

### Dimethyl 2-(3-(((benzyloxy)carbonyl)amino)propyl)malonate (2)

To a stirring solution of NaH (1.1 g, 27.6 mmol, 1.5 eq) in DMF (15 mL) at room temperature was carefully added dimethyl malonate (3.64 g, 27.6 mmol, 1.5 eq) in DMF (5 mL) dropwise. Upon addition, the reaction mixture became a thick suspension. DMF (5 mL) was added to the reaction, and it was stirred for 1 h at RT. Starting material 1 (5.0 g, 18.4 mmol, 1 eq) in DMF (5 mL) was added to the reaction, and the reaction mixture was heated to 75° C. After stirring for 5 hours at 75° C., the reaction was cooled down to RT. The reaction mixture was diluted with EtOAc (100 ml) and washed with water (100 mL). The aqueous phase was extracted with EtOAc (100 mL). The combined organic layers were dried over Na₂SO₄, filtered, and concentrated. The residue was purified by flash chromatography using 0-40% EtOAc in hexanes to give 3.6 g product 2 (60%) as a clear oil. Product was confirmed by LCMS and NMR. HPLC: rt=4.518.

### 2-(3-(((benzyloxy)carbonyl)amino)propyl)malonatic Acid (3)

To a stirring solution of compound 2 (3.6 g, 11.13 mmol, 1 eq) in THF/H₂O (1:1, 50 mL) was added LiOH (0.8 g, 33.4 mmol, 3 eq). The reaction was stirred overnight and then acidified with 1 N HCl to pH=2. The mixture was extracted with EtOAc (60 mL×2), dried over Na₂SO₄, and concentrated to give 3.3 g crude product 3 (100%) as a clear oil which was used in the next step without purification. HPLC: rt=3.40 min.

### Example 3

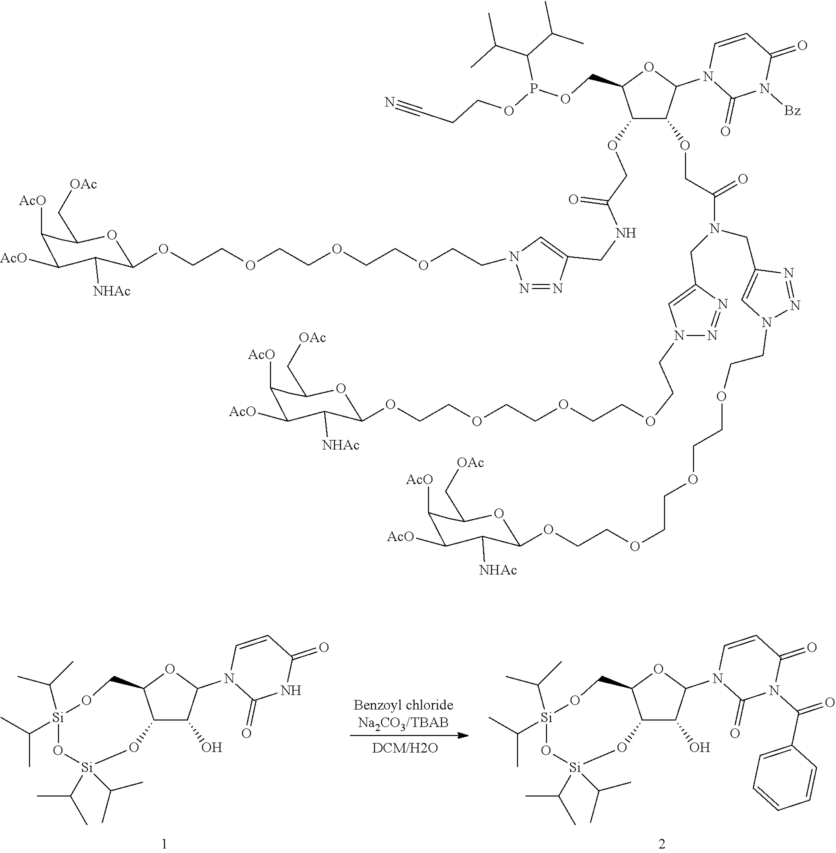

Alcohol 1 (5.0 g, 10.27 mmol, 1 eq), Na₂CO₃ (7.619 g, 71.888 mmol, 7 eq), and tetrabutylammonium bromide (0.132 g, 0.411 mmol, 0.04 eq) were dissolved in a biphasic mixture of CH₂Cl₂ (125 mL) and H₂O (250 mL). Benzoyl chloride (1.550 mL, 13.351 mmol, 1.3 eq) was added to the reaction, and the reaction mixture was vigorously stirred overnight. The reaction mixture was extracted with DCM. The organic phase was dried over anhydrous Na₂SO₄, filtered, and evaporated under reduced pressure. The residue was dissolved in 1,2-dichloroethane (30 mL) and heated at 60° C. for 20 min. The solution was stirred at room temperature overnight, the solvent was removed under reduced pressure, and the residue was purified by column chromatography on silica gel using a gradient 0-40% EtOAc in hexanes to afford 3.4 g (51%) of product (2) as a solid. LCMS: ESI+ m/z=591.1 [M+H]⁺.

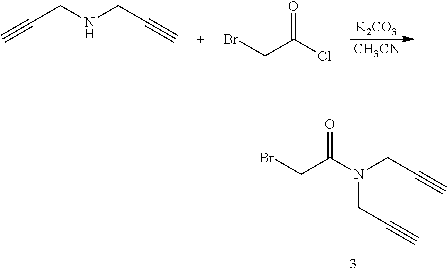

To a suspension dipropargylamine (2 g, 21.48 mmol, 1 eq) and K₂CO₃ (14.84 g, 107.4 mmol, 5 eq) in CH₃CN (50 mL) at 0° C. was added bromoacetyl chloride (1.788 mL, 21.48 mmol, 1 eq). The reaction was stirred for 1 h at 0° C., then at room temperature for 1 h. The reaction was diluted with DCM and washed with water. The organic phase was dried on Na₂SO₄, concentrated, and purified by column chromatography on silica gel using a gradient 0-30% EtOAc in hexanes to afford 2.35 g product 3 (51%) as an oil. LCMS: ESI+ m/z=215.9 [M+H]⁺.

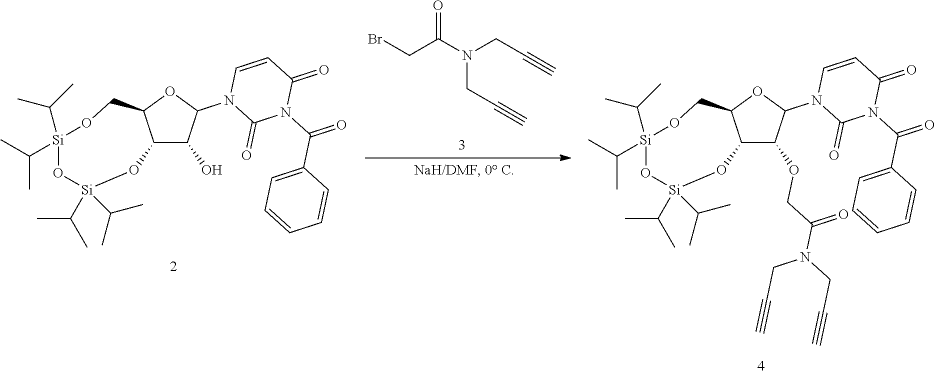

To a stirred solution of alcohol 2 (2.4 g, 4.062 mmol, 1 eq) in DMF (8 mL) at 0° C. was added bromo acetamide 3 (1.304 g, 6.093 mmol, 1.5 eq) followed by NaH (0.211 g, 60% in mineral oil, 2.068 mmol, 1.3 eq). The reaction was stirred at 0° C. for 45 min, then quenched by addition of H₂O. The Reaction mixture was diluted with EtOAc and washed with water. The organic layer was dried over Na₂SO₄, filtered, and concentrated. The residue was purified by flash chromatography using a gradient 0-30% EtOAc in Hexanes to give 2.17 g (74%) product 4 as a white solid. LCMS: ESI+ m/z=725.2 [M+H]⁺.

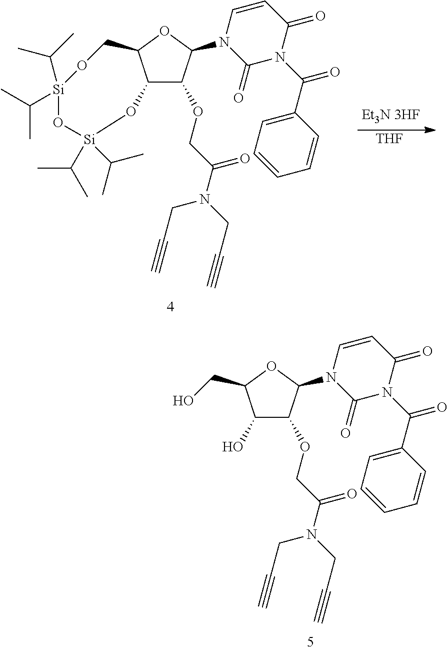 [SMILES: C#CCN(CC#C)C(=O)CO[C@@H]1[C@@H]2O[Si](C(C)C)(C(C)C)O[Si](C(C)C)(C(C)C)OC[C@H]2O[C@H]1n1ccc(=O)n(C(=O)c2ccccc2)c1=O ; C#CCN(CC#C)C(=O)CO[C@@H]1[C@H](O)[C@@H](CO)O[C@H]1n1ccc(=O)n(C(=O)c2ccccc2)c1=O ; "THF"]

To a stirred solution of bis-alkyne 4 (2.91 g, 4.02 mmol, 1 eq) in THF (30 mL) was added Et₃N·3HF (3.276 mL, 20.098 mmol, 5 eq). The reaction was stirred overnight at room temperature then quenched by addition of MeOH. The reaction mixture was diluted with EtOAc and washed with aqueous saturated NaHCO₃ then brine. The organic layer was dried over Na₂SO₄, filtered, and concentrated. The residue was purified by flash chromatography using a gradient 0-5% MeOH in EtOAc to give 1.85 g alcohol 5 (95%) as a white solid. LCMS: ESI+ m/z=482.0 [M+H]⁺.

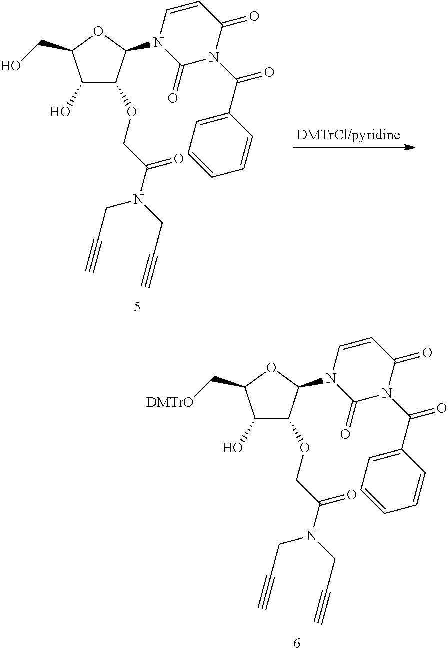

To a stirred solution of the alcohol 5 (1.85 g, 3.842 mmol, 1 eq) in pyridine (30 mL) was added DMTrCl (1.693 mg, 4.995 mmol, 1.3 eq). The reaction was stirred at room temperature overnight. The reaction was quenched by addition of MeOH and the mixture was concentrated under reduced pressure. The residue obtained was partitioned between EtOAc and aqueous saturated NaHCO₃. The organic layer was dried over Na₂SO₄, filtered, and concentrated. The residue obtained was purified by column chromatography on silica gel using a gradient 0-50% EtOAc in hexanes to afford 2.48 g DMT ether 6 (82%) as a white solid. LCMS: ESI+ m/z=806.0 [M+Na]⁺.

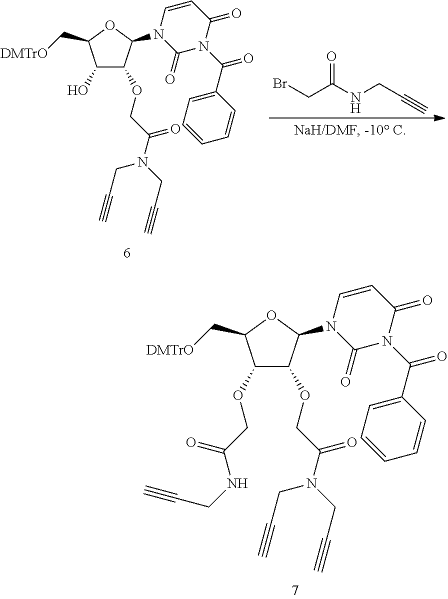

To a stirred solution of DMT ether 6 (780 mg, 0.995 mmol, 1 eq) in DMF (4 mL) at −10° C. was added NaH (52 mg, 60% in mineral oil, 1.294 mmol, 1.3 eq) followed by 2-bromo-N-(prop-2-yn-1-yl)acetamide (263 mg, 1.493 mmol, 1.5 eq) in DMF (1 mL). The reaction was stirred at −10° C. for 20 mins then quenched by addition of H₂O. The reaction mixture was partitioned between EtOAc and water. The aqueous phase was extracted with EtOAc, and the combined organic layers were dried over Na₂SO₄, filtered, and concentrated. The residue obtained was purified by column chromatography on silica gel using a gradient 0˜60% EtOAc in hexanes to afford 380 mg (43%) of product 7 as a white solid. LCMS: ESI+ m/z=901.7 [M+Na]⁺.

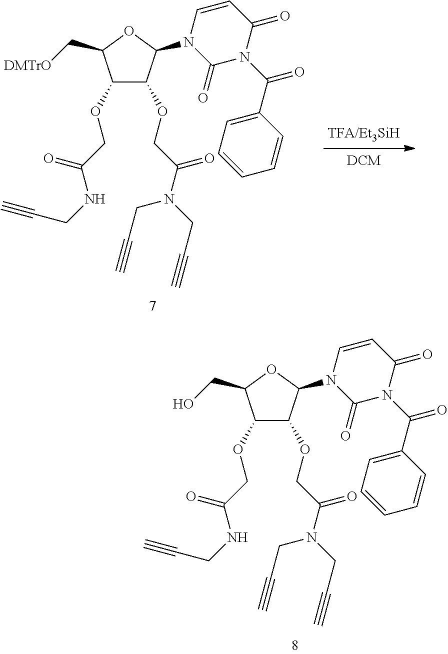

To a solution of DMT ether 7 (0.38 g, 0.432 mmol, 1.00 eq) in DCM (5 mL) at room temperature was added TFA (0.076 mL, 0.994 mmol, 2.30 eq) followed by Et₃SiH (0.069 mL, 0.432 mmol, 1 eq). The reaction was stirred at room temperature for 2 h then quenched with aqueous saturated NaHCO₃. The product was extracted with DCM, and the organic layers were combined, dried over Na₂SO₄, filtered, and concentrated. The residue was purified by column chromatography on silica gel using a gradient 0-10% MeOH in DCM to afford 179 mg alcohol 8 (71%). LCMS: ESI+ m/z=577.0 [M+H]⁺.

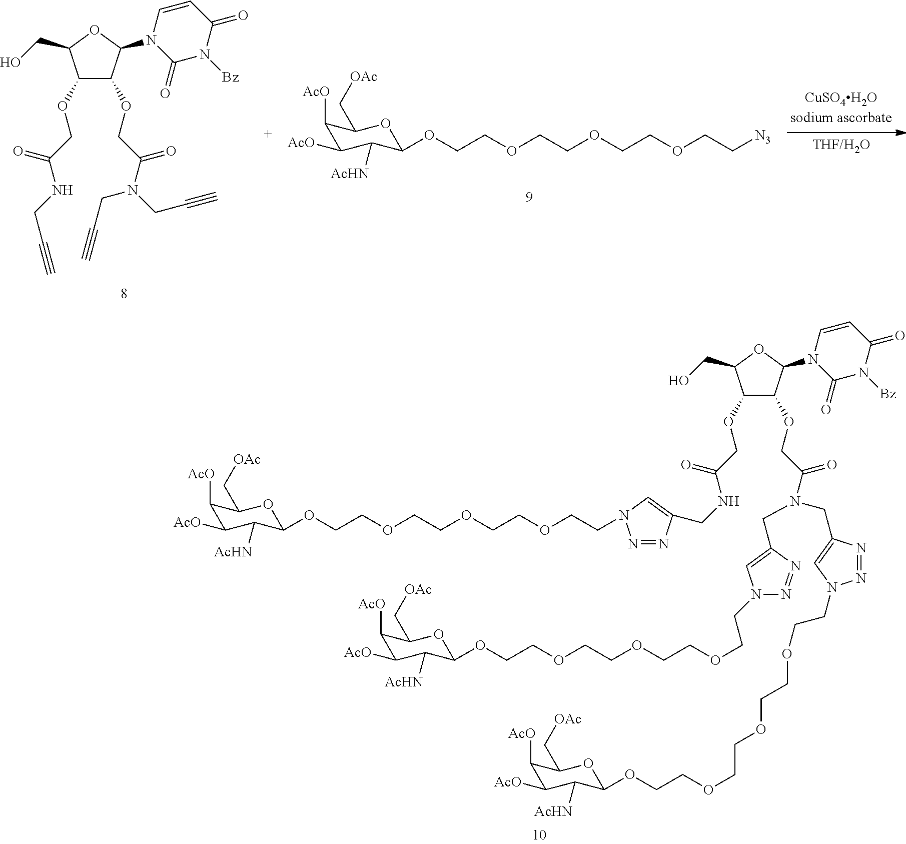

To a solution of alcohol 8 (179 mg, 0.31 mmol, 1 eq) and azide 9 (0.562 g, 1.025 mmol, 3.3 eq) in THF (8 mL) at 0° C. was added a solution of CuSO₄·5H₂O (0.233 g, 0.931 mmol, 3 eq) in water (2 mL) followed by a solution of sodium ascorbate (0.215 g, 1.087 mmol, 3.5 eq) in water (2 mL). After stirring for 5 min at 0° C., the reaction was warmed to room temperature then stirred for 45 mins at room temperature. The mixture was diluted with DCM then washed with NaHCO₃. The aqueous phase was extracted with DCM, and the organic layers were combined, dried over Na₂SO₄, filtered, and concentrated. The residue obtained was purified by column chromatography on silica gel using a gradient 0-20% MeOH in DCM to afford 398 mg of triazole 10 (58%) as a solid. MS: m/z=2243.5 [M+Na]⁺.

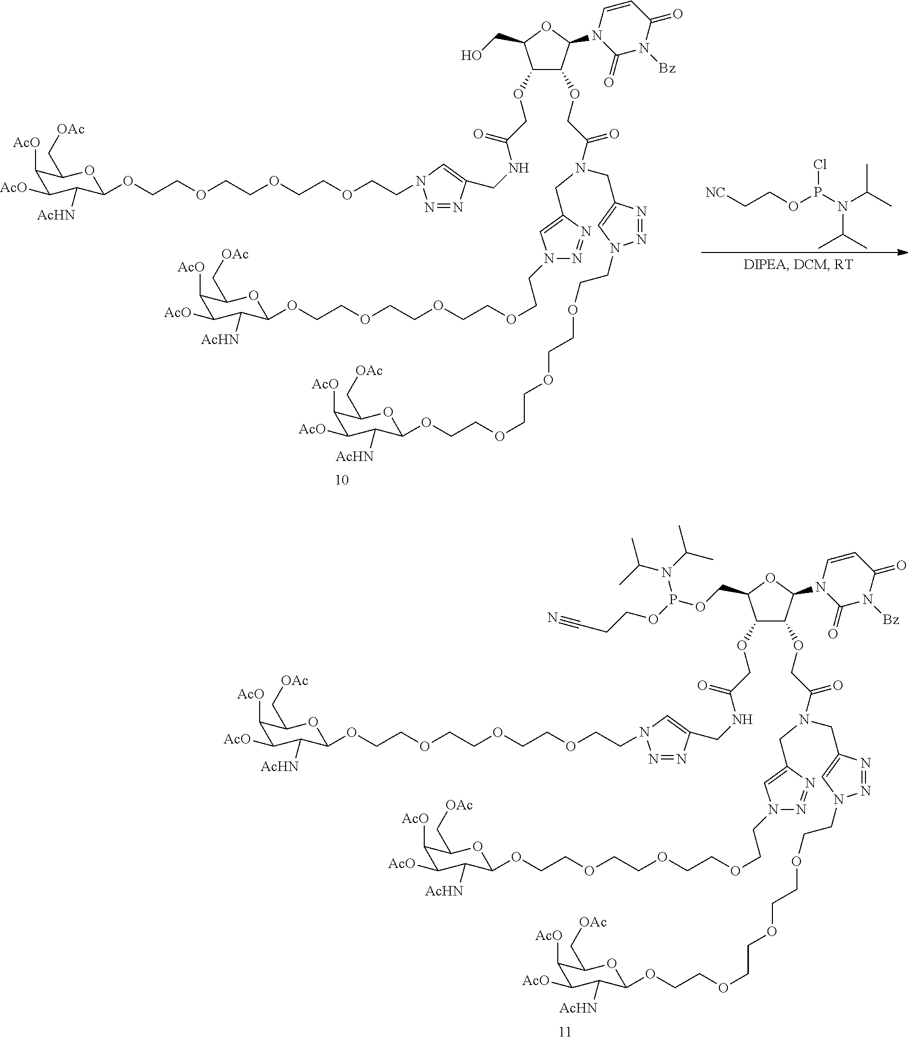

To a stirred solution of triazole 10 (100 mg, 0.045 mmol, 1 eq) and diisopropylethylamine (0.051 mL, 0.135 mmol, 6.5 eq) in anhydrous DCM (2 mL) under Argon was added 2-cyanoethyl N,N-diisopropyl chlorophosphoramidite (0.030 mL, 0.293 mmol, 3 eq) dropwise. The reaction mixture was stirred at room temperature for 30 mins, then quenched with aqueous saturated NaHCO₃ solution. The product was extracted with DCM, and the combined organic phases were washed with brine, dried over Na₂SO₄, filtered, then concentrated. The residue obtained was purified by flash chromatography using a gradient 0-5% MeOH in DCM in the presence of base to afford 71 mg of phosphoramidite 11 (65%). MS: m/z=2443.6 [M+H]⁺.

### Example 4

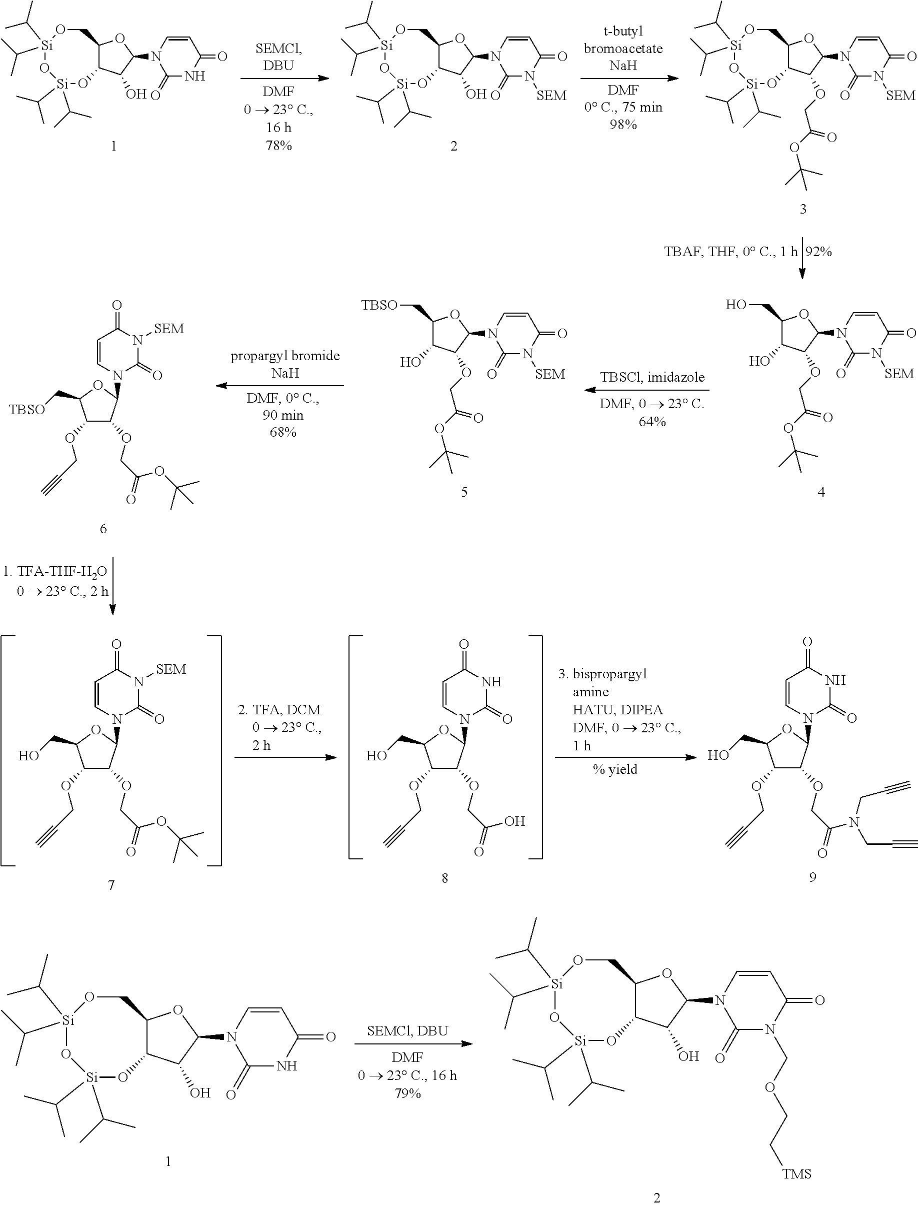

SEMCl (3.3 mL, 18.5 mmol, 1.2 equiv) was added dropwise to a solution of alcohol 1 (7.5 g, 15.4 mmol, 1 equiv) and DBU (3.5 mL, 23 mmol, 1.5 equiv) in DMF (50 mL) at 0° C. The reaction mixture was stirred overnight at room temperature then diluted with ethyl acetate and hexanes and poured into cold water (500 mL). The layers were partitioned, and the organic layer was successively washed with water (2×300 mL) then brine (200 mL). The organic layer was dried over anhydrous sodium sulfate, filtered, and concentrated under reduced pressure. The residue obtained was purified by flash column chromatography on silica gel (50 g) using a gradient 0-25% ethyl acetate in hexanes to afford compound 2 (7.5 g, 79%). LCMS: ESI+ m/z=639.4 [M+Na]⁺.

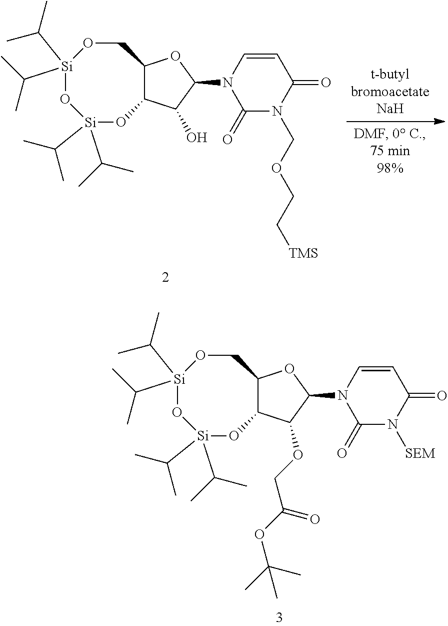 [SMILES: CC(C)[Si]1(C(C)C)OC[C@H]2O[C@@H](n3ccc(=O)n(COCC[Si](C)(C)C)c3=O)[C@H](O)[C@@H]2O[Si](C(C)C)(C(C)C)O1 ; CC(C)[Si]1(C(C)C)OC[C@H]2O[C@@H](n3ccc(=O)n(COCC[Si](C)(C)C)c3=O)[C@H](OCC(=O)OC(C)(C)C)[C@@H]2O[Si](C(C)C)(C(C)C)O1]

Alcohol 2 (2.7 g, 4.38 mmol, 1 equiv) was taken up in DMF (29 mL) and cooled to 0° C. t-Butyl bromoacetate (1.9 mL, 13 mmol, 3 equiv) was added, followed by sodium hydride (219 mg, 5.5 mmol, 1.25 equiv). The reaction mixture was stirred at 0° C. for 1 h then residual base was quenched at 0° C. by the addition of methanol (1.5 mL). The product was extracted with ethyl acetate and the organic layer was washed with water then brine. The organic phase was dried over sodium sulfate, filtered, then concentrated under reduced pressure. The residue obtained was purified by flash column chromatography on silica gel (50 g) using a gradient 0-20% ethyl acetate in hexanes to afford ester 3 (3.13 g, 98%) as an oil. LCMS: ESI+ m/z=753.4 [M+Na]⁺.

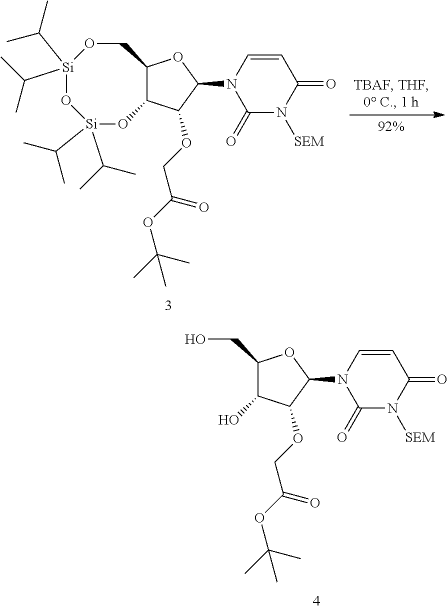 [SMILES: CC(C)[Si]1(C(C)C)OC[C@H]2O[C@@H](n3ccc(=O)n(COCC[Si](C)(C)C)c3=O)[C@H](OCC(=O)OC(C)(C)C)[C@@H]2O[Si](C(C)C)(C(C)C)O1 ; CC(C)(C)OC(=O)CO[C@@H]1[C@H](O)[C@@H](CO)O[C@H]1n1ccc(=O)n(COCC[Si](C)(C)C)c1=O]

TBAF (1.0 M in THF, 12.7 mL, 3.9 mmol, 3 equiv) was added dropwise to a solution of disiloxane 3 (3.1 g, 4.24 mmol, 1 equiv) in THF (42 mL, 0.1 M) at 0° C. After 2 h at 0° C., the reaction mixture was poured into a saturated solution of sodium bicarbonate (200 mL) and was extracted with DCM (2×150 mL). The combined organic extracts were washed with water (200 mL) then brine (150 mL). The combined organic layer was dried over anhydrous sodium sulfate, filtered, then concentrated under reduced pressure. The residue obtained was purified by flash column chromatography on silica gel (25 g) using a gradient 0-70% ethyl acetate in hexanes to afford diol 4 (1.82 g, 88%) as an oil. LCMS: ESI+ m/z=511.0 [M+Na]⁺.

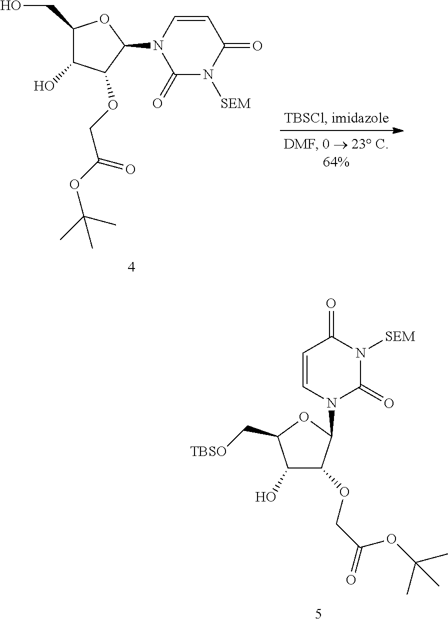 [SMILES: CC(C)(C)OC(=O)CO[C@@H]1[C@H](O)[C@@H](CO)O[C@H]1n1ccc(=O)n(COCC[Si](C)(C)C)c1=O ; CC(C)(C)OC(=O)CO[C@@H]1[C@H](O)[C@@H](CO[Si](C)(C)C(C)(C)C)O[C@H]1n1ccc(=O)n(COCC[Si](C)(C)C)c1=O]

TBSCl (666 mg, 4.4 mmol, 1.2 equiv) was added as a solid in one portion to a solution of diol (4) (1.8 g, 3.7 mmol, 1 equiv) and imidazole (501 mg, 7.4 mmol, 2 equiv) in DMF (18 mL) at 0° C. The reaction mixture was stirred at room temperature for 2.5 h, diluted with ethyl acetate, then poured into stirring cold water (200 mL). The organic layer was washed with water (2×200 mL) then brine (150 mL). The organic layer was dried over anhydrous sodium sulfate, filtered, then concentrated under reduced pressure. The residue obtained was purified by flash column chromatography on silica gel (50 g) using a 0-25% ethyl acetate in hexanes to afford alcohol 5 (1.94 g, 87%) as an oil. LCMS: ESI+ m/z=625.2 [M+Na]⁺.

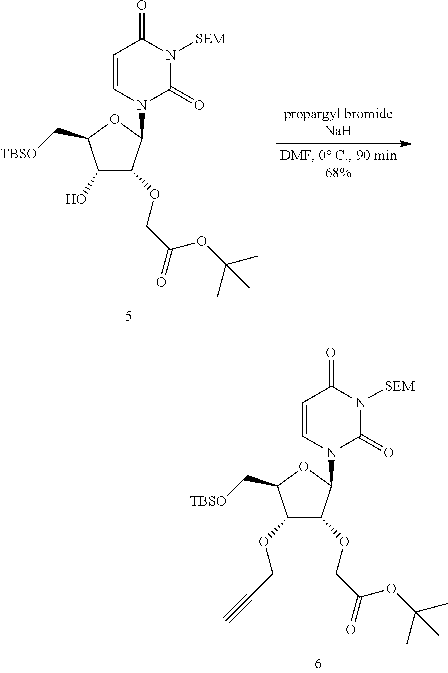 [SMILES: CC(C)(C)OC(=O)CO[C@@H]1[C@H](O)[C@@H](CO[Si](C)(C)C(C)(C)C)O[C@H]1n1ccc(=O)n(COCC[Si](C)(C)C)c1=O ; C#CCO[C@H]1[C@@H](OCC(=O)OC(C)(C)C)[C@H](n2ccc(=O)n(COCC[Si](C)(C)C)c2=O)O[C@@H]1CO[Si](C)(C)C(C)(C)C]

Alcohol 5 (1.94 g, 3.14 mmol, 1 equiv) was taken up in anhydrous DMF (21 mL) and cooled to 0° C. Propargyl bromide (80% w/w in toluene, 0.7 mL, 6.29 mmol, 2 equiv) was added, followed by NaH (157 mg, 3.93 mmol, 1.25 equiv) as a solid in one portion. After 90 minutes at 0° C., additional propargyl bromide (80% w/w in toluene, 0.35 mL, 3.1 mmol, 1 equiv) was added, followed by NaH (63 mg, 1.57 mmol, 0.5 equiv). After an additional 1 h, the reaction mixture was diluted with ethyl acetate and washed with water (2×200 mL) then brine (150 mL). The organic layer was dried over anhydrous sodium sulfate, filtered, then concentrated under reduced pressure. The residue obtained was purified by flash column chromatography on silica gel (25 g) using a gradient of 0-15% ethyl acetate in hexanes to afford alkyne (6) (1.38 g, 68% yield) as an oil. LCMS: ESI+ m/z=663.3 [M+Na]⁺.

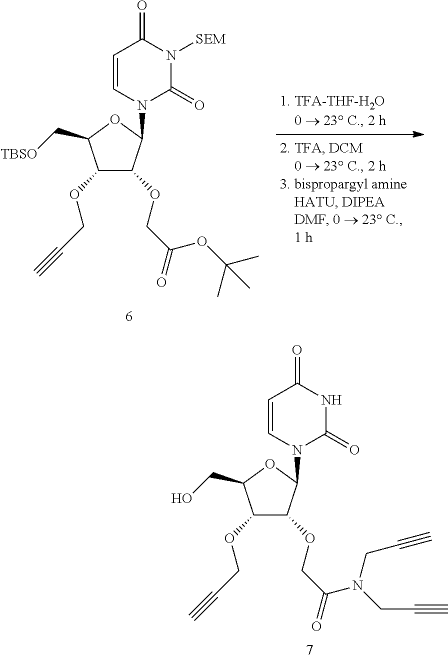 [SMILES: C#CCO[C@H]1[C@@H](OCC(=O)OC(C)(C)C)[C@H](n2ccc(=O)n(COCC[Si](C)(C)C)c2=O)O[C@@H]1CO[Si](C)(C)C(C)(C)C ; C#CCO[C@H]1[C@@H](OCC(=O)N(CC#C)CC#C)[C@H](n2ccc(=O)[nH]c2=O)O[C@@H]1CO]

TFA (2.5 mL) was added dropwise to a solution of t-butyl ester (6) (0.98 g, 1.53 mmol, 1 equiv) in THF (10 mL) and H₂O (2.5 mL) at 0° C. The reaction mixture was stirred for 1.5 h at room temperature then concentrated. The residue obtained was dissolved in dichloromethane (10 mL), cooled to 0° C., then TFA (5 mL) was added. After 1.5 h, the reaction mixture was concentrated under reduced pressure and the residue obtained was diluted with DCM (2 mL) and toluene (2 mL), then concentrated under reduced pressure. The residue obtained was dissolved in DMF (5 mL), cooled to 0° C., then DIPEA (1.3 mL, 7.65 mmol, 5 equiv), dipropargyl amine (0.8 mL, 7.65 mmol, 5 equiv) and HATU (1.098 g, 2.89 mmol, 1.9 equiv) were added consecutively. The reaction mixture was stirred at room temperature for 75 mins then the reaction mixture was diluted with ethyl acetate and 1 N HCl (50 mL). The mixture was stirred vigorously for 5 minutes and then the organic phase was separated. The aqueous layer was further extracted with ethyl acetate. The combined organic extracts were washed with water (2×50 mL) then brine (50 mL), dried over anhydrous sodium sulfate, filtered, and concentrated under reduced pressure. The residue obtained was purified by flash column chromatography on silica gel (25 g) using a gradient 0-95% ethyl acetate in hexanes to afford amide 7 (532 mg, 84%) as a sticky solid. LCMS: ESI+ m/z=416.1 [M+H]⁺.

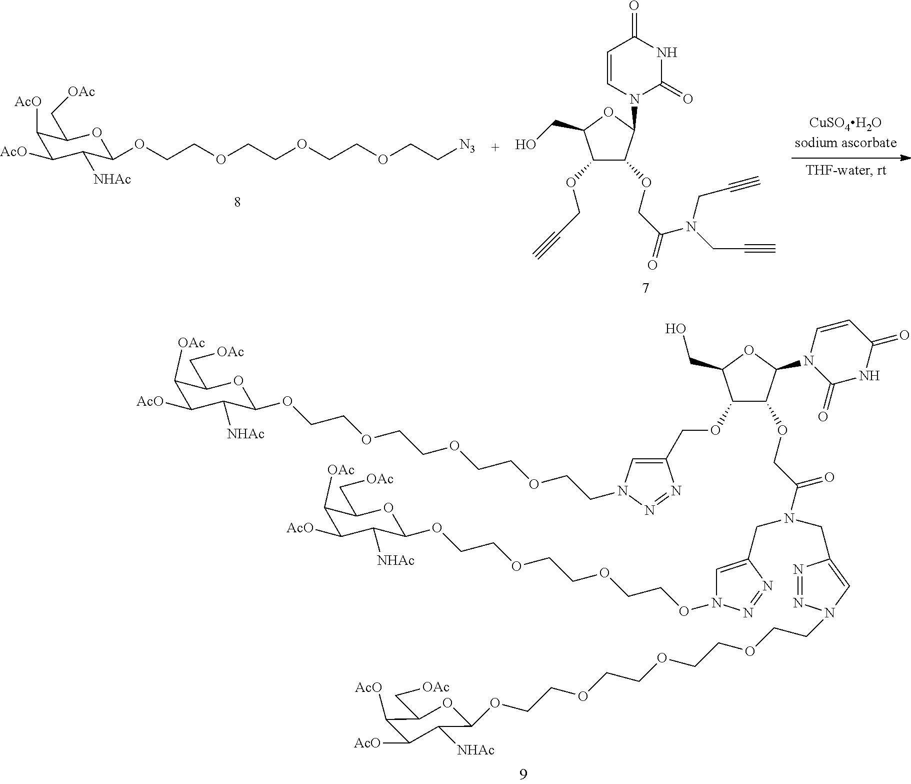 [SMILES: CC(=O)NC1C(OCCOCCOCCOCCN=[N+]=[N-])OC(COC(C)=O)C(OC(C)=O)C1OC(C)=O ; C#CCO[C@H]1[C@@H](OCC(=O)N(CC#C)CC#C)[C@H](n2ccc(=O)[nH]c2=O)O[C@@H]1CO ; CC(=O)NC1C(OCCOCCOCCOCCn2cc(CO[C@H]3[C@@H](OCC(=O)N(Cc4cn(CCOCCOCCOCCOC5OC(COC(C)=O)C(OC(C)=O)C(OC(C)=O)C5NC(C)=O)nn4)Cc4cn(OCCOCCOCCOC5OC(COC(C)=O)C(OC(C)=O)C(OC(C)=O)C5NC(C)=O)nn4)[C@H](n4ccc(=O)[nH]c4=O)O[C@@H]3CO)nn2)OC(COC(C)=O)C(OC(C)=O)C1OC(C)=O]

A solution of sodium ascorbate (90 mg, 0.455 mmol, 3.5 equiv) in water (1.5 mL) and a solution of copper sulfate pentahydrate (97 mg, 0.39 mmol, 3 equiv) in water (1.5 mL) were successively added dropwise to the solution of alkyne 7 and azide 8 in THF at 0° C. After 5 mins, the reaction mixture was stirred at room temperature for 3 h then diluted with DCM (50 mL) and washed with aqueous saturated sodium bicarbonate (50 mL). The aqueous layer was extracted with DCM (3×25 mL), the combined organic extracts were dried over anhydrous sodium sulfate then filtered over celite. The filtrate was concentrated under reduced pressure and the residue obtained was purified by flash column chromatography on silica gel (5 g) using a gradient 0-75% methanol in ethyl acetate to afford tris-triazole 9 (120 mg, 45%) as a sticky solid. LCMS: ESI+ m/z=1031.0 [(M+2H)/2]⁺.

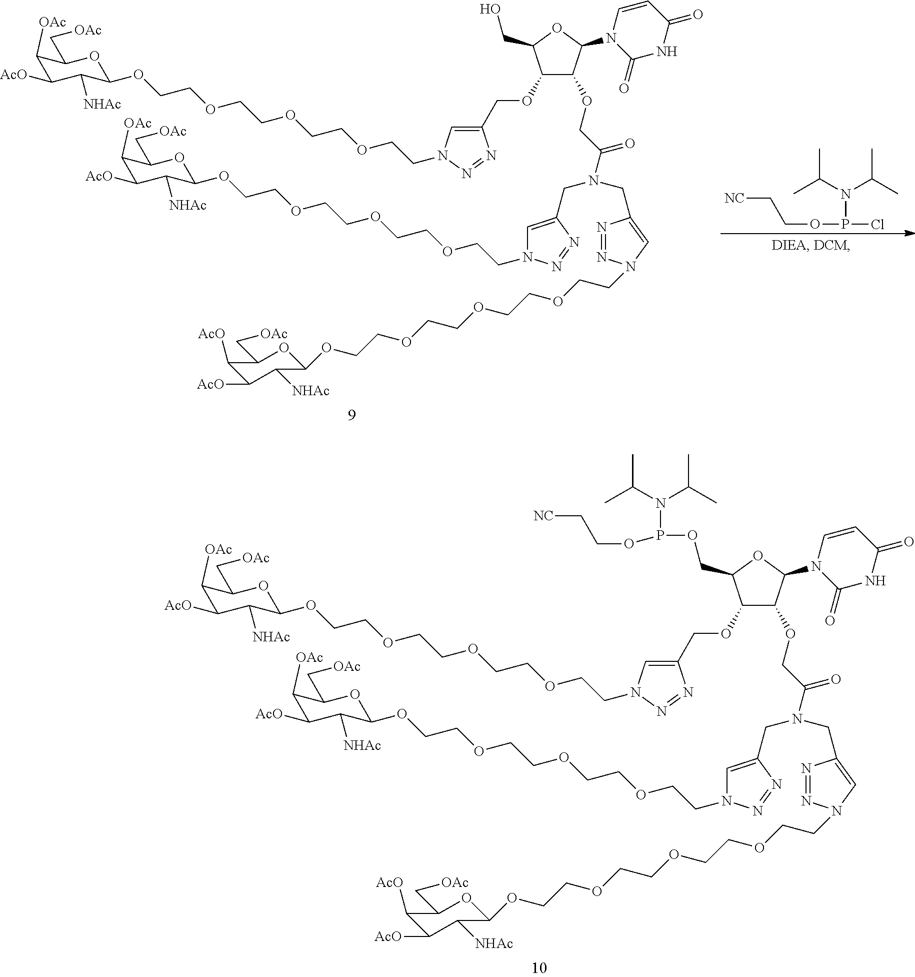

Compound 10 was synthesized from compound 9 following the same procedure as Example 3. LCMS: ESI+ m/z=1089.4 [(M−NH(iPr)₂+H₂O+2H)/2]⁺.

### Example 5

### Synthesis of Asymmetric Tri-Antennary 3′-Triazole GalNAc-Linkers:

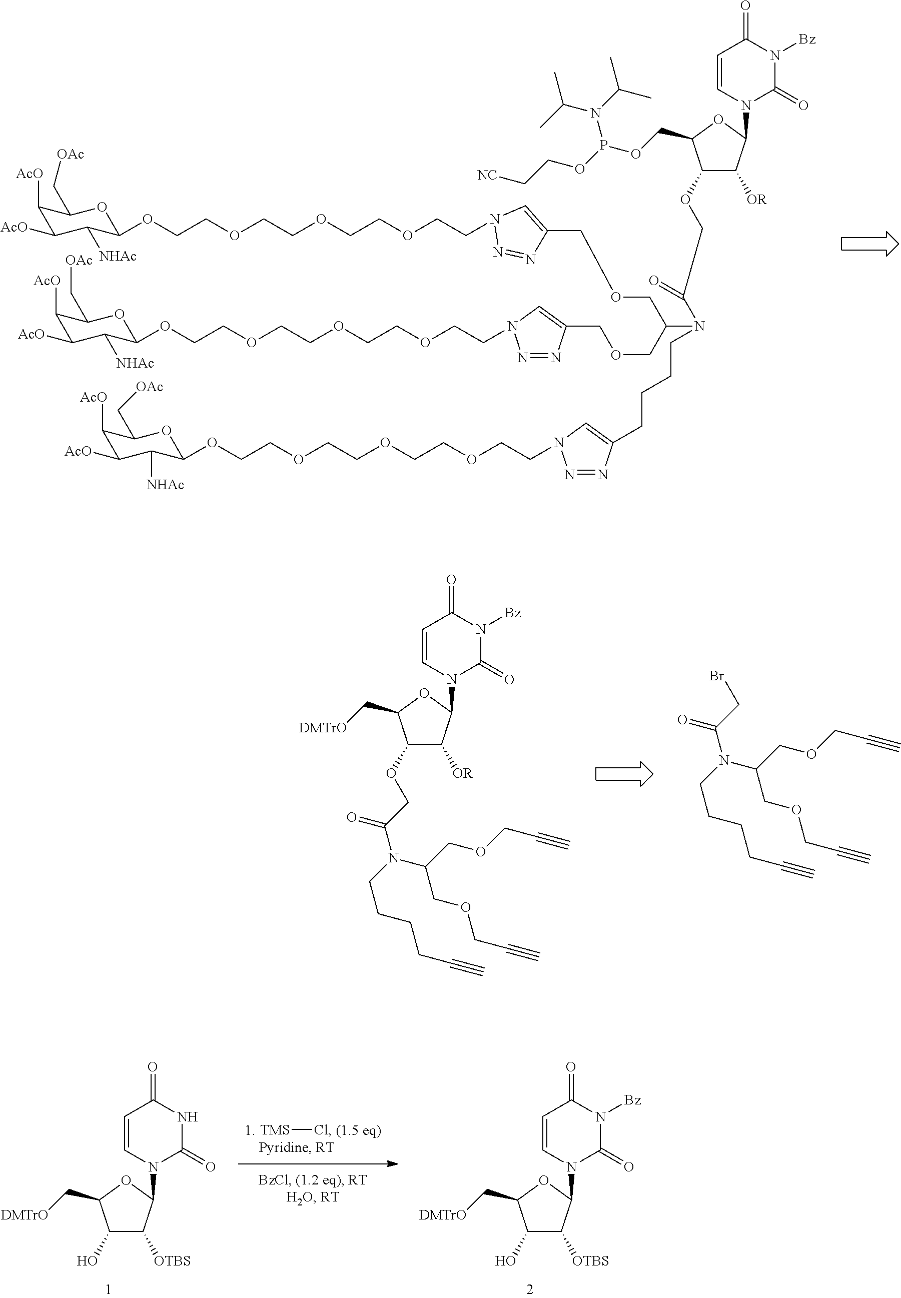

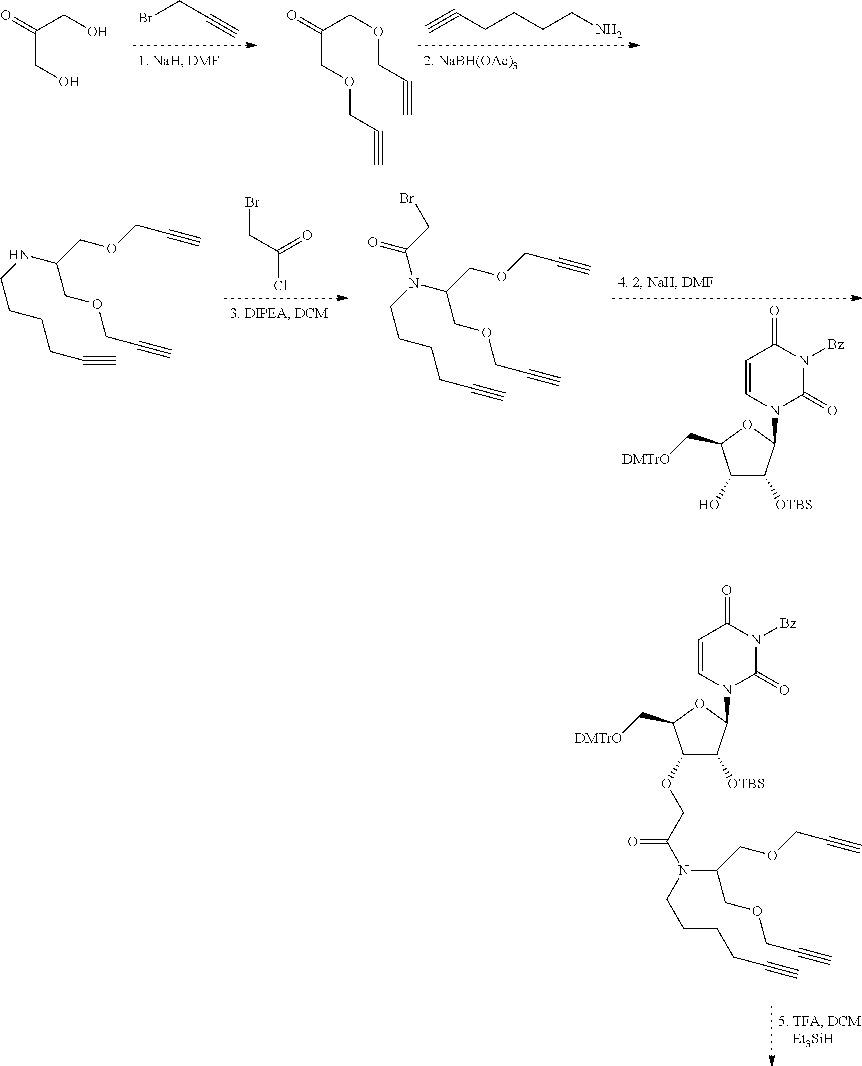

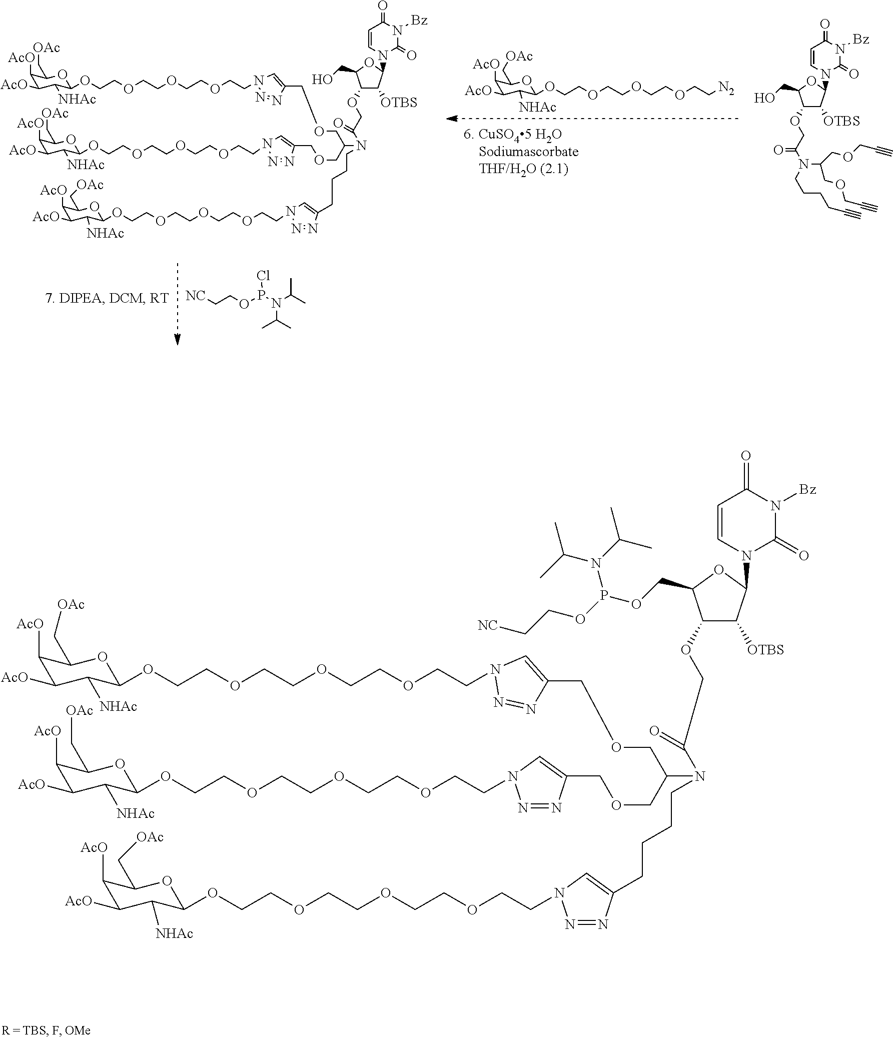

### Synthesis of Asymmetric Tri-Antennary 5′-GalNAc-Triazole Linkers:

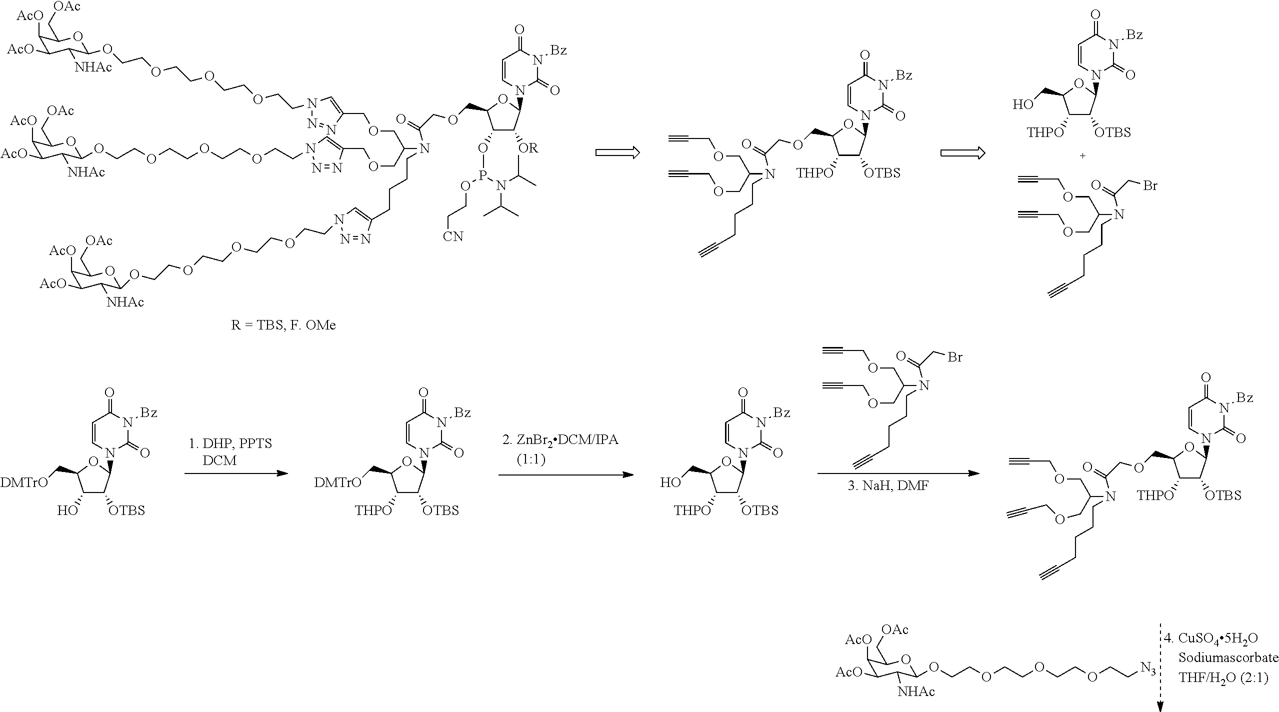

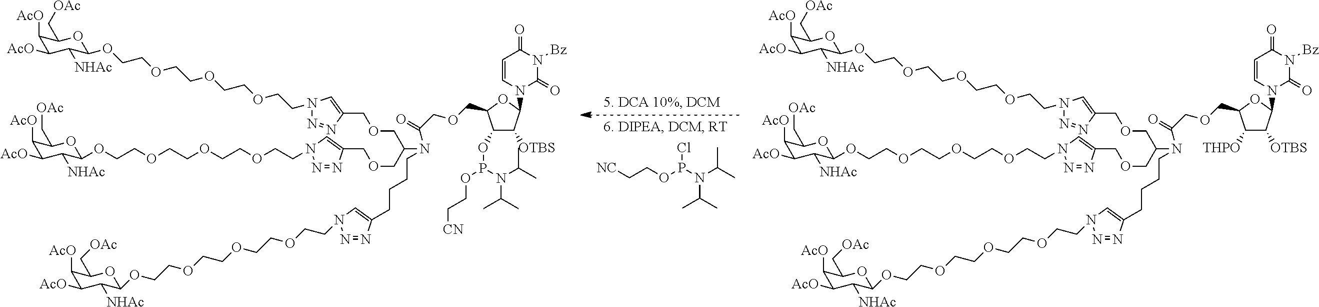

### Synthesis of Asymmetric Tri-Antennary 3′-GalNAc-Triazole & Amide Linkers:

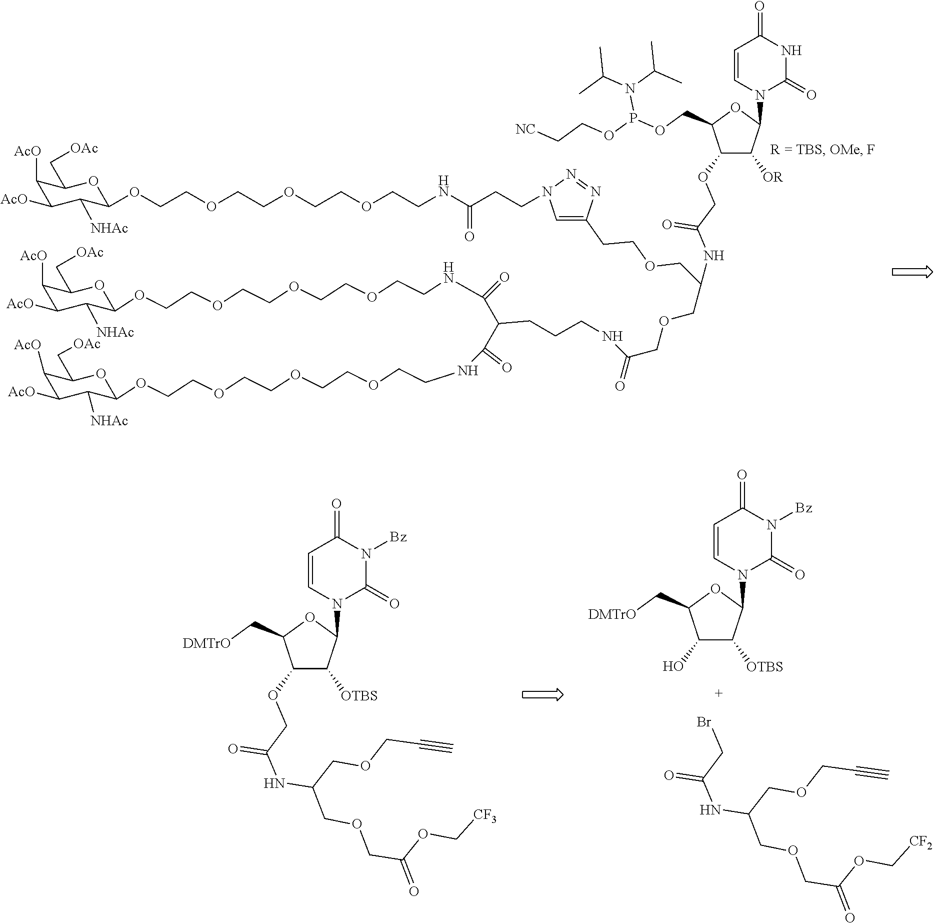

Exemplary compounds falling within the scope of the present disclosure could be synthesized according to the following scheme:

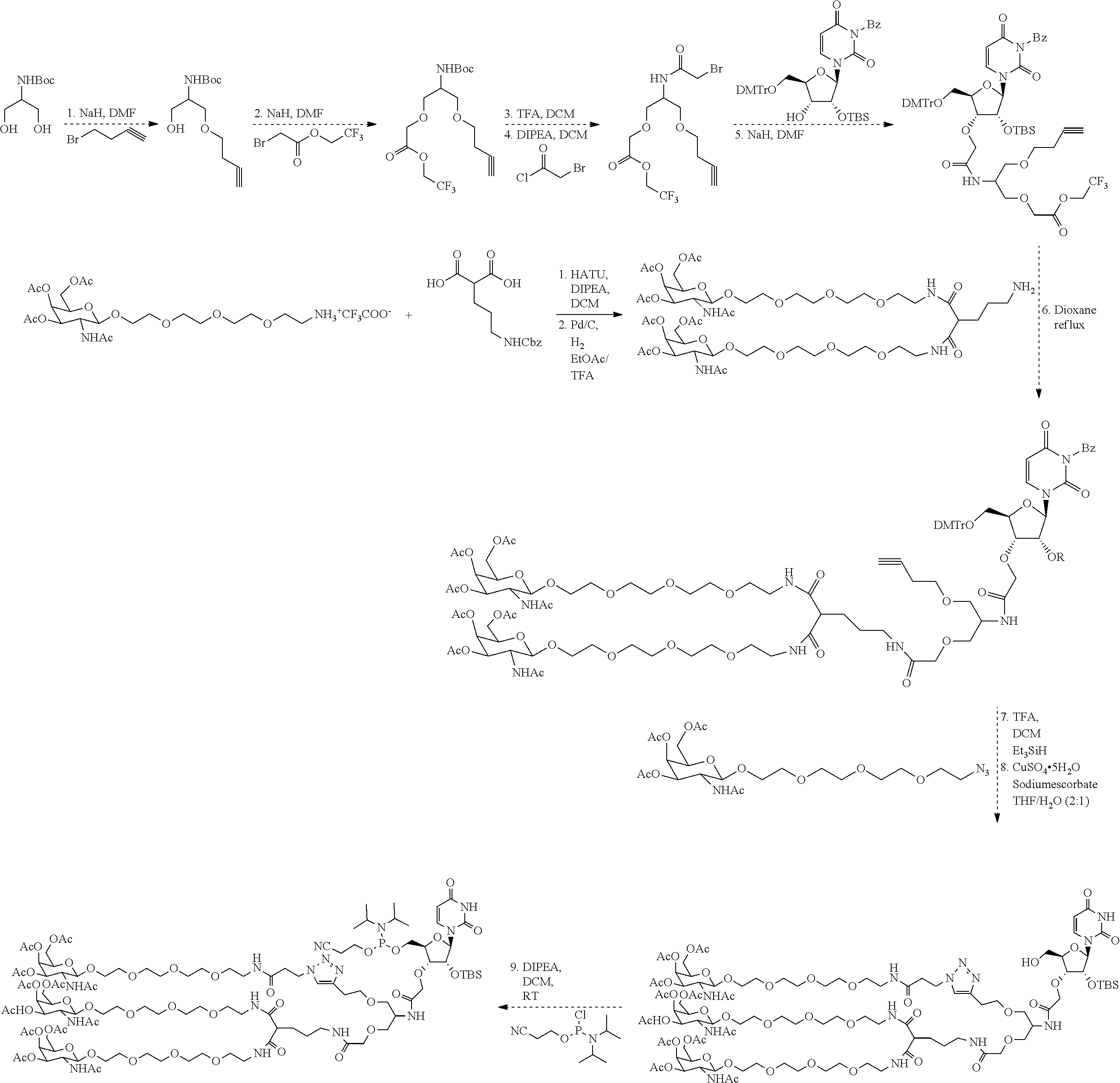

### Synthesis of Asymmetric Tri-Antennary 3′-Triazole GalNAc-Linkers:

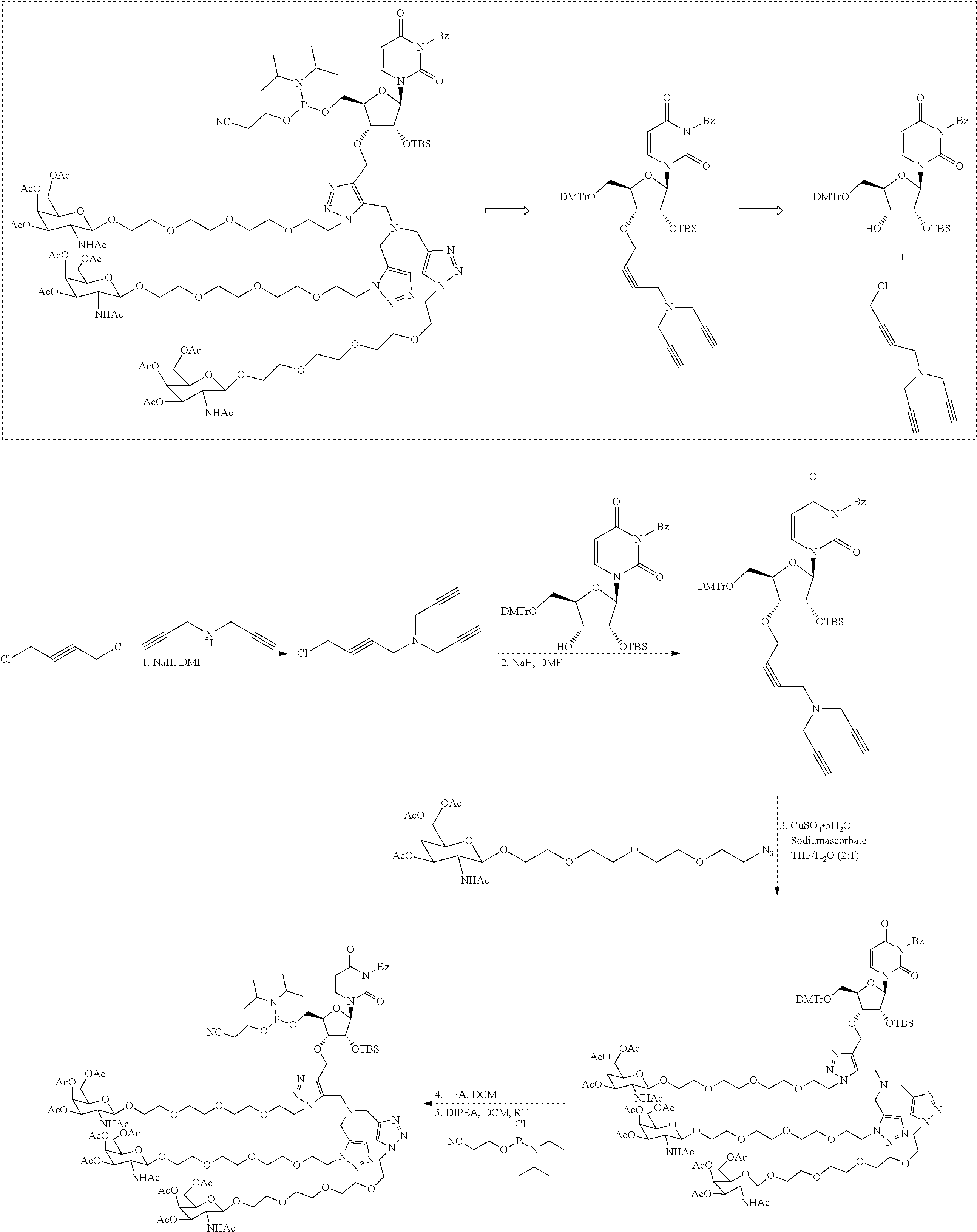

### Synthesis of Asymmetric Tri-Antennary 3′-GalNAc Amide Linkers:

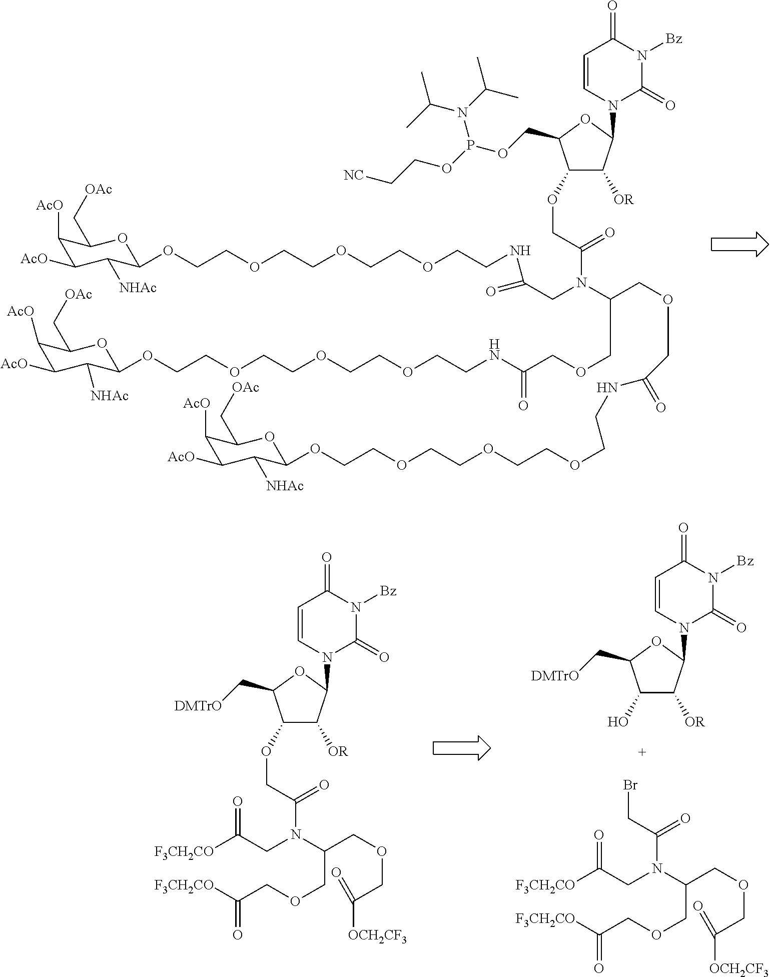

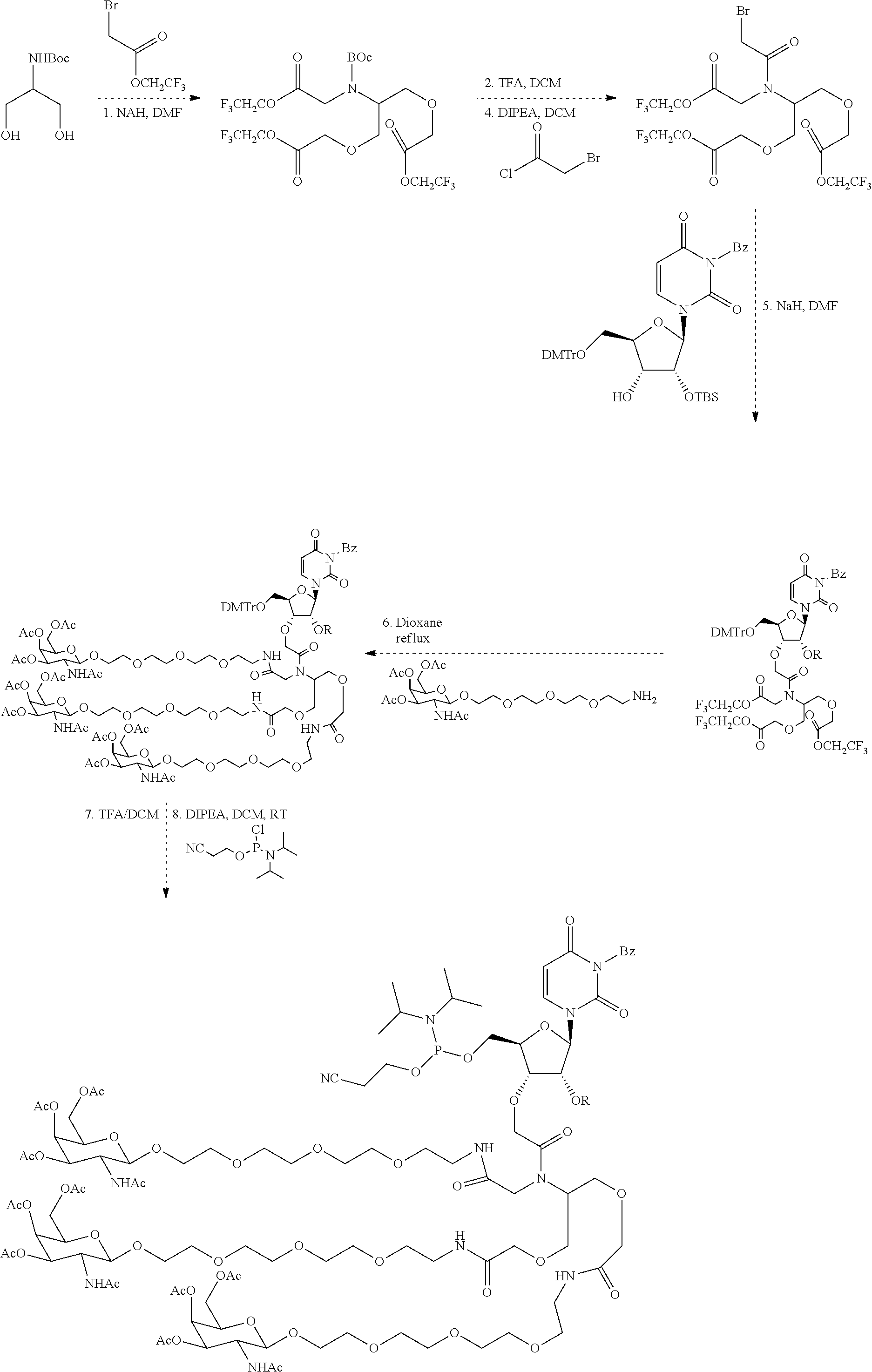

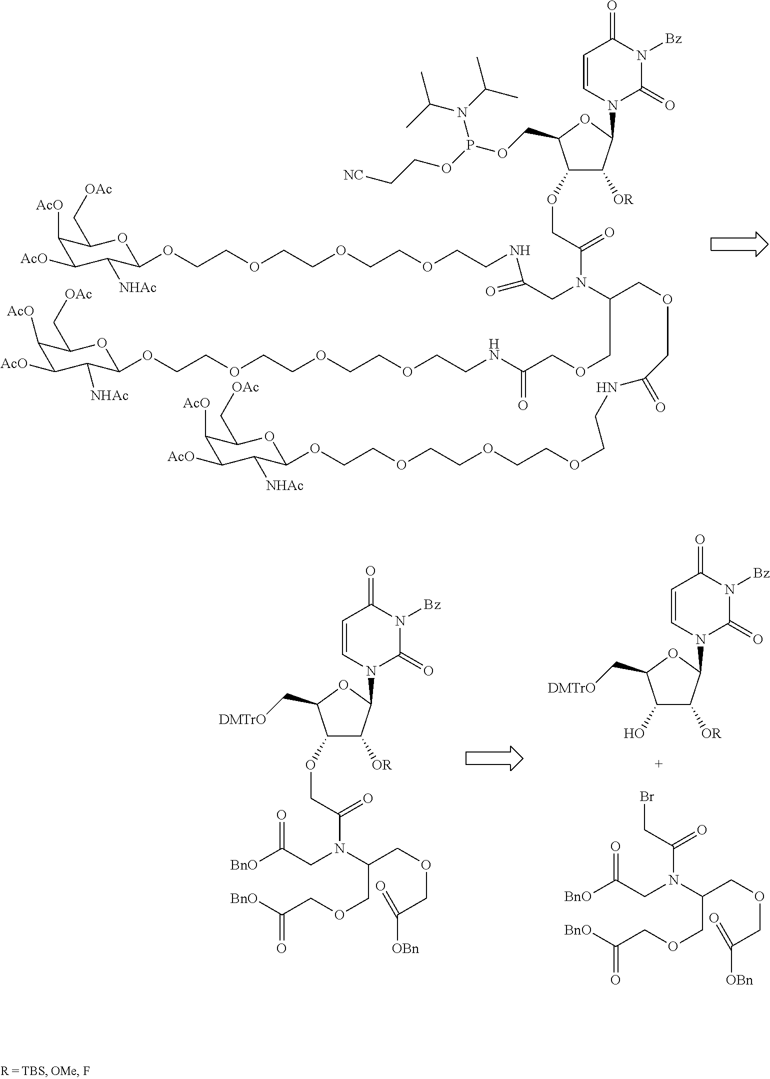

Exemplary compounds falling within the scope of the present disclosure could be synthesized according to the following scheme:

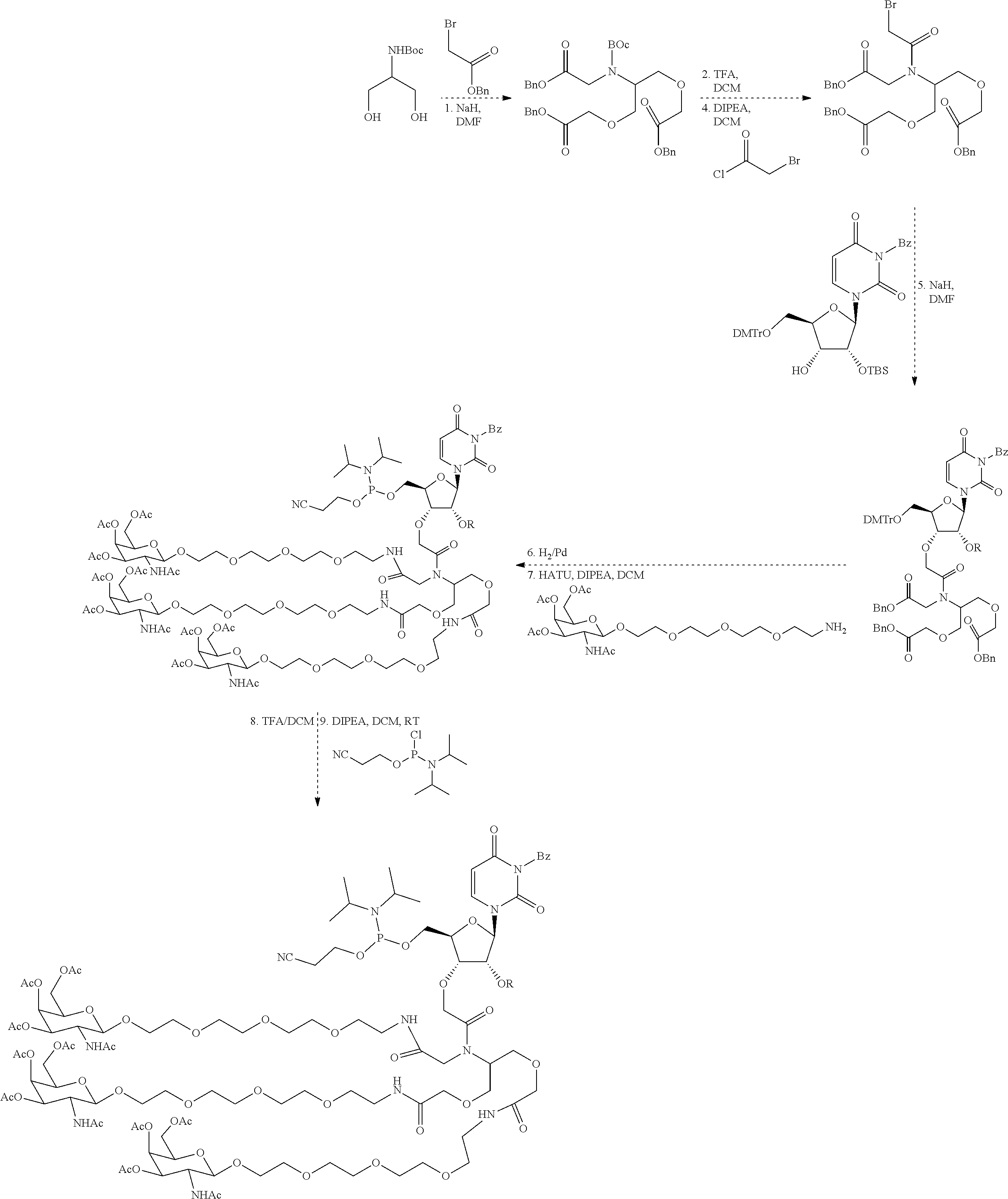

### Example 6

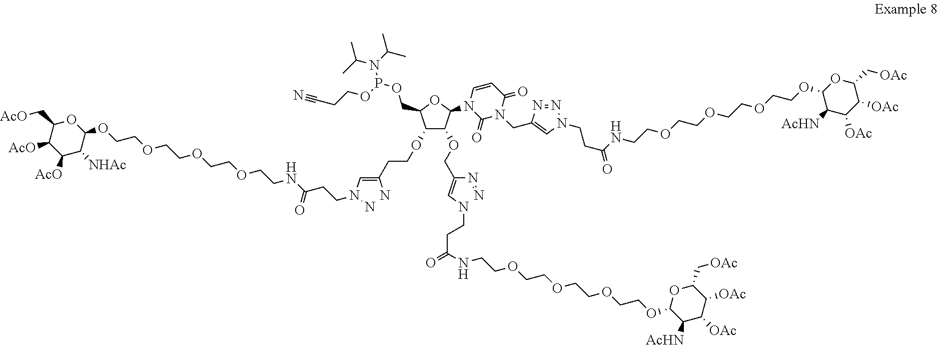 [SMILES: CC(=O)N[C@H]1[C@H](OCCOCCOCCOCCNC(=O)CCn2cc(CCO[C@H]3[C@@H](OCc4cn(CCC(=O)NCCOCCOCCOCCO[C@@H]5O[C@H](COC(C)=O)[C@H](OC(C)=O)[C@H](OC(C)=O)[C@H]5NC(C)=O)nn4)[C@H](n4ccc(=O)n(Cc5cn(CCC(=O)NCCOCCOCCOCCO[C@@H]6O[C@H](COC(C)=O)[C@H](OC(C)=O)[C@H](OC(C)=O)[C@H]6NC(C)=O)nn5)c4=O)O[C@@H]3COP(OCCC#N)N(C(C)C)C(C)C)nn2)O[C@H](COC(C)=O)[C@H](OC(C)=O)[C@@H]1OC(C)=O]

### Intermediate 2 (INT-2).

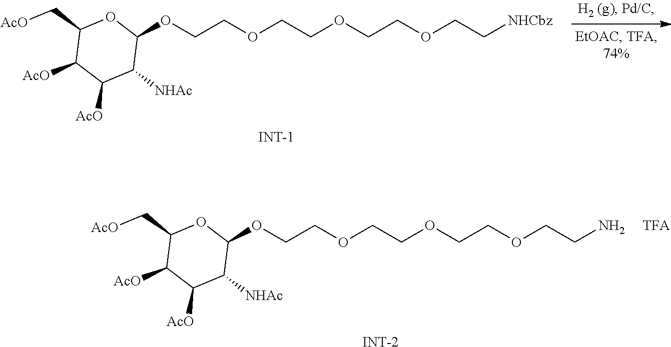 [SMILES: CC(=O)N[C@H]1[C@H](OCCOCCOCCOCCNC(=O)OCc2ccccc2)O[C@H](COC(C)=O)[C@H](OC(C)=O)[C@@H]1OC(C)=O ; "INT-1" ; CC(=O)N[C@H]1[C@H](OCCOCCOCCOCCN)O[C@H](COC(C)=O)[C@H](OC(C)=O)[C@@H]1OC(C)=O ; "INT-2" ; "TFA"]

To a solution of INT-1 (8.5 g, 12.94 mmol, 1 eq) in EtOAc (200 mL) and TFA (3 mL) under argon was added Pd/C (1.377 g, 10 wt %, 1.294 mmol, 0.1 eq). The round-bottom flask was flushed with H₂ gas, and the suspension was then stirred overnight under H₂ (1 atm). The catalyst was filtered off through celite, and the filtrate was concentrated. The residue obtained was purified by silica-gel column chromatography using a gradient 0-20% MeOH in DCM to give INT-2 (5.9 g, 74%).

### Intermediate 3 (INT-3).

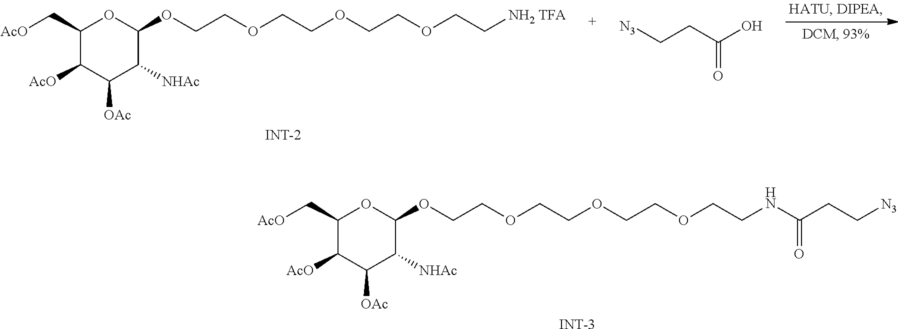

To a solution of INT-2 (3.5 g, 5.649 mmol, 1 eq) and 3-azidopropionic acid (0.975 g, 8.474 mmol, 1.5 eq) in DCM (50 mL) at 0° C. were successively added HATU (3.222 g, 8.474 mmol, 1.5 eq) then DIPEA (9.84 mL, 56.492 mmol, 10 eq) dropwise. The reaction was stirred at 0° C. for 5 min then at rt for 1 h. The reaction was quenched by addition of water and the product was partitioned between DCM and H₂O. The aqueous phase was extracted with DCM, the combined organic layers were dried over Na₂SO₄, filtered, and concentrated. The residue obtained was purified by silica-gel column chromatography using a gradient 0-10% MeOH in EtOAc to give INT-3 (3.27 g, 93%).

### Intermediate 5 (INT-5).

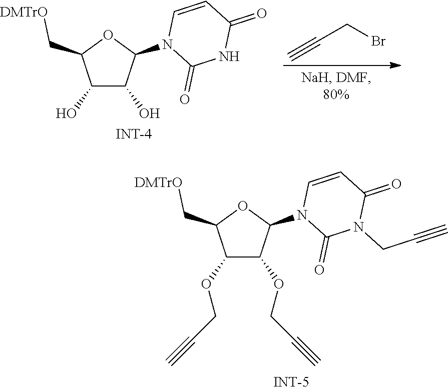

To a stirred solution of INT-4 (1 g, 1.832 mmol, 1 eq) at 0° C. in anhydrous DMF (15 ml) was added NaH 60 w % (220 mg 5.495 mmol, 3 eq). The reaction mixture was stirred at 0° C. for 30 mins, and then propargyl bromide (0.68 mL, 4.579 mmol, 2.5 eq) was added dropwise and the mixture was stirred for 12 h at room temperature. The reaction mixture was quenched with water (20 mL), extracted with ethyl acetate (3×50 mL), dried (Na₂SO₄), and then concentrated. The residue obtained was purified by silica gel column chromatography using a gradient 0-50% ethyl acetate in hexane as an eluent to INT-S (919 mg 80%) as a solid. LCMS ESI+: m/z=683 [M+Na]+.

### Intermediate 6 (INT-6).

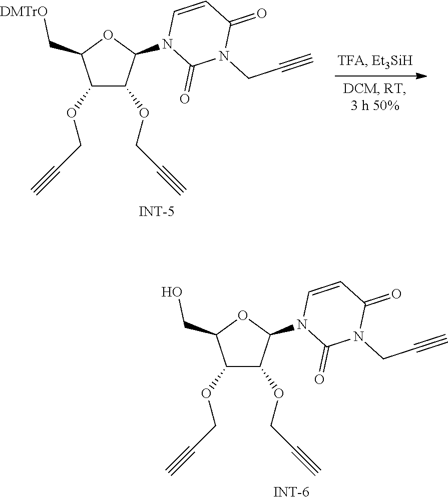

To a solution of INT-5 (400 mg, 0.606 mmol, 1.00 eq.) in DCM (10 mL) at room temperature were successively added TFA (0.1 mL, 1.33 mmol, 2.20 eq) and Et₃SiH (0.1 mL, 0.667 mmol, 1.1 eq.). The reaction mixture was stirred at room temperature for 5 h then diluted with DCM (50 mL). The organic phase was washed with NaHCO₃ (50 mL) then brine (50 mL), dried (Na₂SO₄), filtered, and concentrated. The residue obtained was purified by silica-gel column chromatography using a gradient 0-100% EtOAc in hexanes to afford INT-6 (200 mg, 50%) as a solid. LCMS ESI+: m/z=359 [M+H]+.

### Intermediate 7 (INT-7).

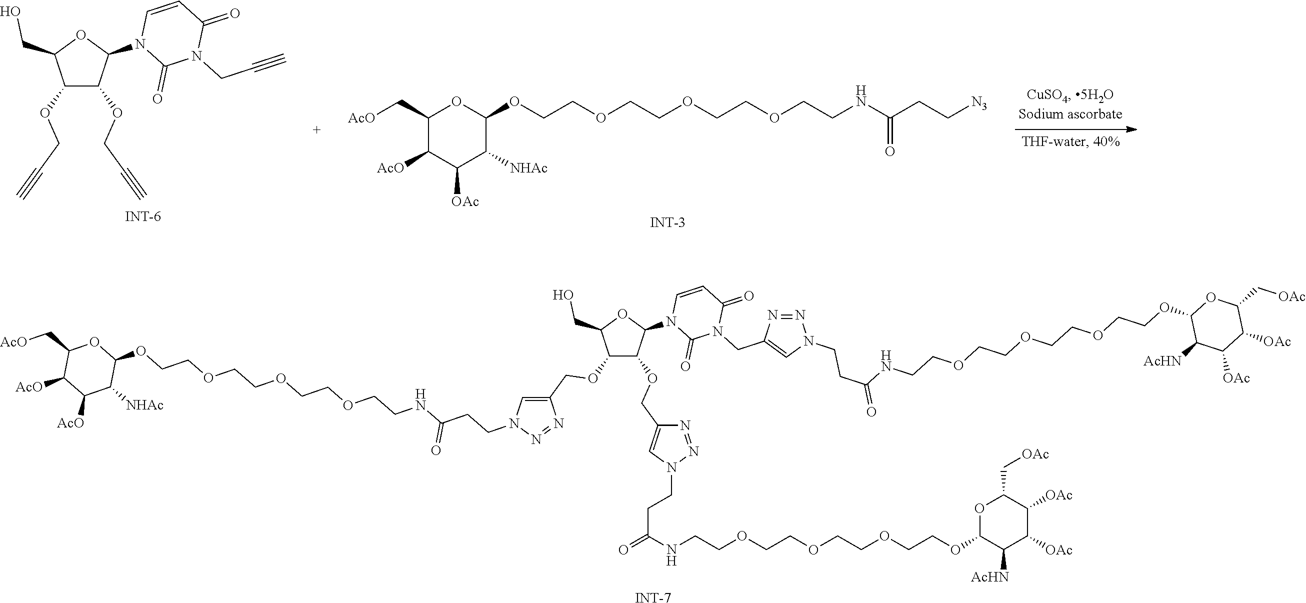

To a stirred mixture of INT-6 (8 mg, 0.022 mmol, 1 eq) and INT-3 (62 mg, 0.101 mmol, 4.5) in THF (1 mL) at 0° C. was added a solution of CuSO₄·5H₂O (17 mg, 0.067 mmol, 3 eq) in water (0.25 mL), followed by a solution of sodium ascorbate (15 mg, 0.078 mmol, 3.5 eq) in water (0.25 mL). The mixture was stirred for 1 h at room temperature, diluted with aqueous saturated NaHCO₃ (10 mL), and then extracted with DCM (2×20 mL). The organic phases were combined, washed with brine (20 mL), dried (Na₂SO₄), filtered, and concentrated. The crude residue obtained was purified by silica-gel column chromatography using a gradient 0-30% MeOH in DCM to afford INT-7 (20 mg 40%) as a solid. LCMS ESI+: m/z=2239 [M+Na]+.

### Example 6

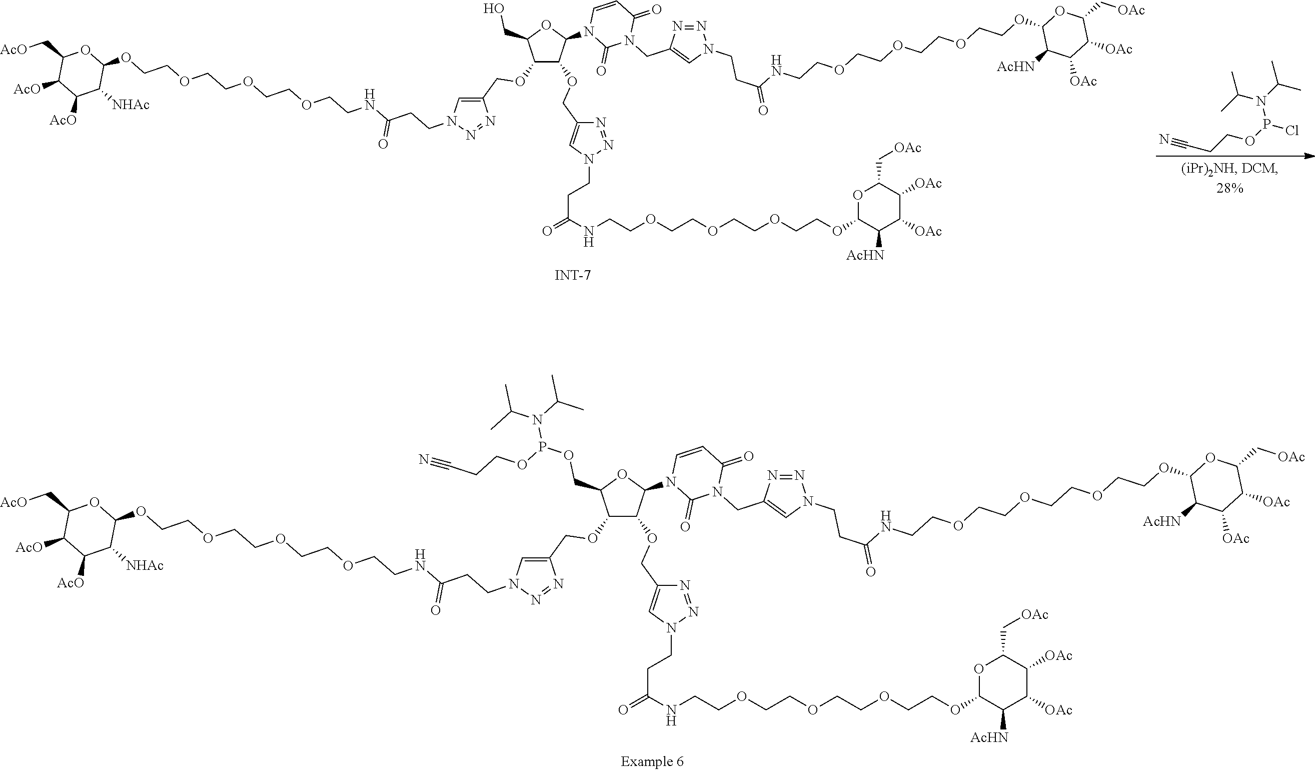

To a solution of INT-7 (70 mg, 0.032 mmol, 1 eq) in DCM (10 ml) at 0° C. were successively added diisopropylethylamine (0.013 mL, 0.076 mmol, 2.4 eq) followed by yanoethyl N,N-diisopropyl chlorophosphoramidite (0.008 mL, 0.038 mmol, 1.2 eq) dropwise. The reaction mixture was stirred at room temperature for 2 hours, then another portion of diisopropylethylamine (0.013 mL, 0.076 mmol, 2.4 eq) followed by cyanoethyl N, N-diisopropyl chlorophosphoramidite (0.008 mL, 0.038 mmol, 1.2 eq) were added dropwise. The mixture was stirred for 24 h at room temperature, diluted with aqueous saturated NaHCO₃ solution (20 mL), and the product was extracted with DCM (2×50 mL). The organic phases were dried (Na₂SO₄), filtered, and concentrated. The residue obtained was purified by silica-gel column chromatography using a gradient 0-30% MeOH in DCM to afford Example 6 (10 mg, 26%) as a solid. LCMS ESI+: m/z=2439 [M+Na]+. ¹H and ³¹P NMR are consistent with the structure of the desired product.

### Example 7

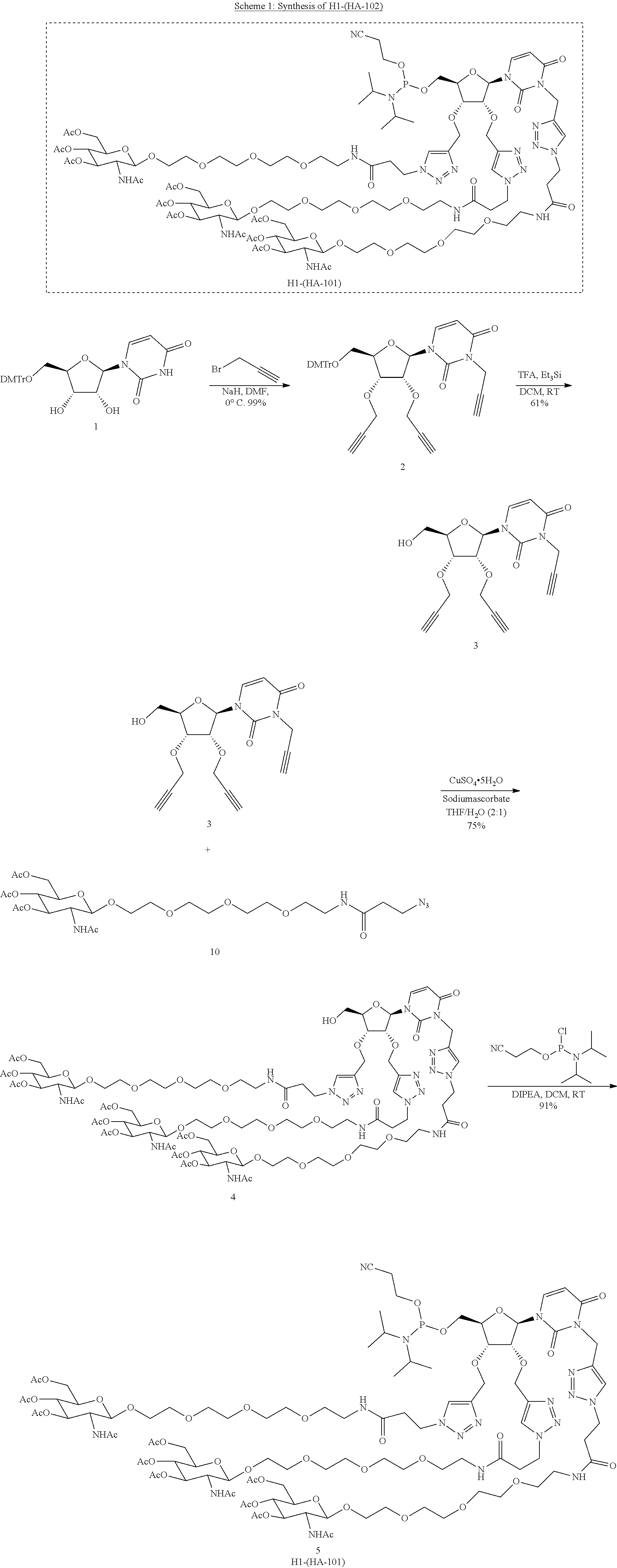

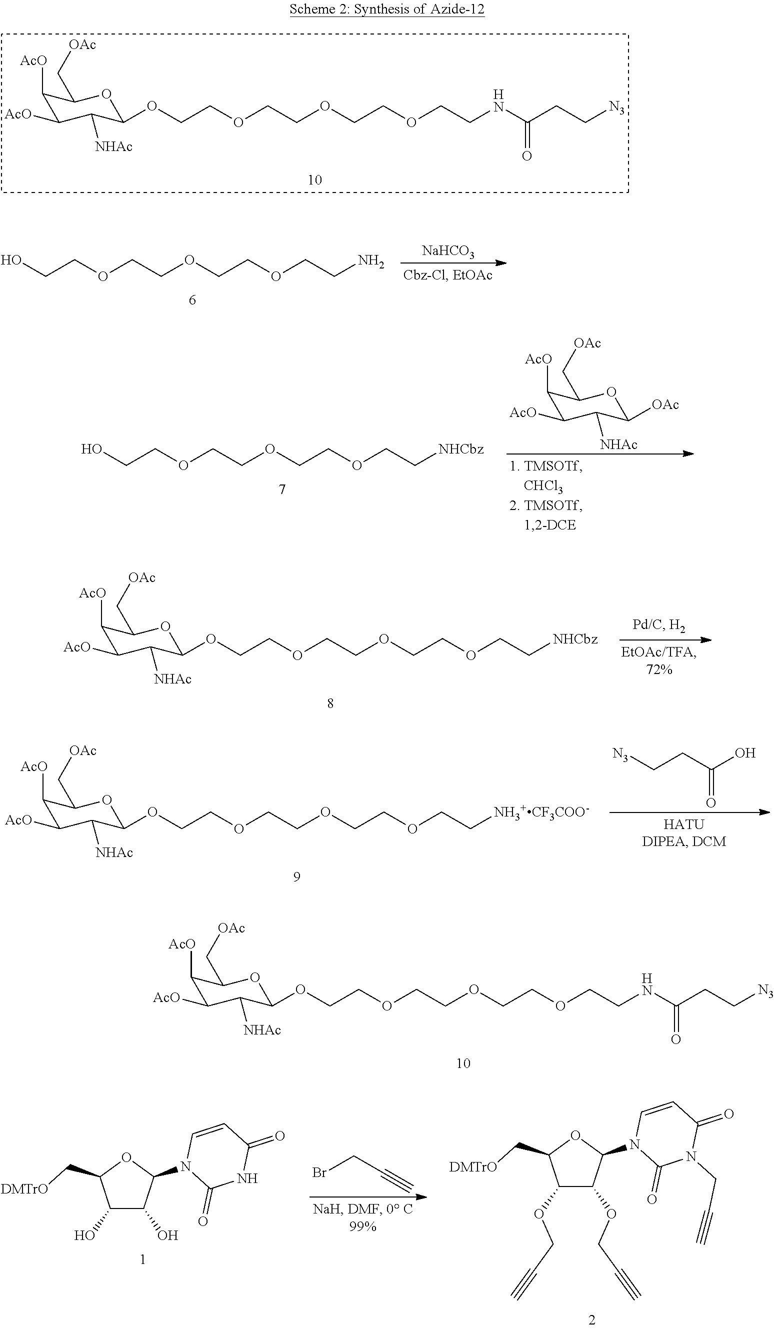

To a stirred and cooled (0° C.) solution of compound 1 (10.08 g, 18.462 mmol, 1 eq) in anhydrous DMF (100 ml) was added NaH 60 w % (2.58 mg 64.615 mmol, 3 eq). The reaction mixture was stirred at 0° C., for 30 min. Propargylbromide (8.236 mL, 55.38 mmol, 3 eq) was added dropwise, stirring was continued for 2 h at 0° C. The reaction mixture was quenched with water (200 mL), extracted with Ethyl acetate (200 mL), washed with water 2×100 mL and Brine 100 mL, dried (Na2SO₄) and concentrated, and the residue was purified by silica gel column chromatography using 0-70% Ethyl acetate/hexane as an eluent. Pure fractions were combined and concentrated to obtained tri-propargyl 2 (12.1 g 99%) as a white solid. Product was confirmed by NMR and LCMS (m/z 684 M+Na).

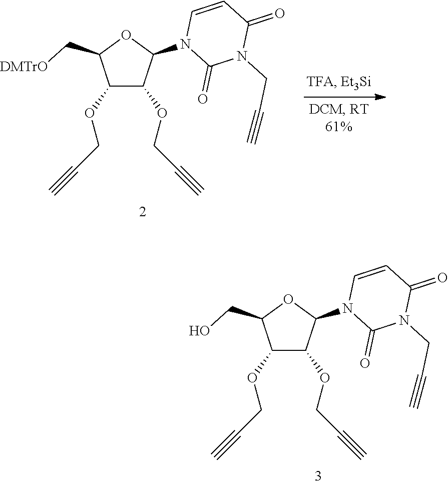

To a solution of DMT ether 2 (4.1 g, 6.212 mmol, 1.00 eq.) in DCM (30 mL) was added TFA (1.09 mL, 14.288 mmol, 2.30 eq.). The color of the solution turned to red. Et₃SiH (1.09 mL, 6.833 mmol, 1.1 eq.) was added at room temperature. The reaction mixture was stirred at room temperature for 5 h. LCMS showed complete deprotection. The reaction mixture was diluted with aq. saturated NaHCO₃ (100 ml), extracted with DCM (2×200 ml), and the organic phase was washed with NaHCO₃ (50 ml), brine (100 ml), dried over Na₂SO₄, filtered and concentrated. The residue was purified by silica-gel column chromatography using a gradient 0-100% EtOAc in hexanes as eluent to afford the desired product 3 as a solid (1.35 g, 61%). LCMS ESI+ m/z 359 [M+H]⁺.

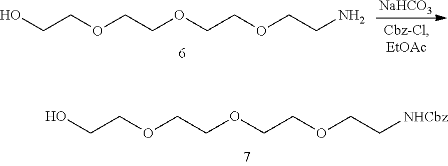

To a mixture of commercially available amino alcohol 6 (50.0 g, 258.75 mmol, 1 eq) in EtOAc (250 mL) 250 mL H₂O was added NaHCO₃ (30.4 g, 362.25 mmol, 1.4 eq), followed by addition of CbzCl (36.8 mL, 258.75 mmol, 1 eq) at rt. The mixture was stirred at rt overnight. Work-up: Organic layer was separated, aqueous phase was extracted with EtOAc (80 ml×2), and dried over MgSO₄. The residue was purified by flash chromatography using 30% to 50% EtOAc/Hept to give compound 7 (72 g, 85% yield) as a syrup.

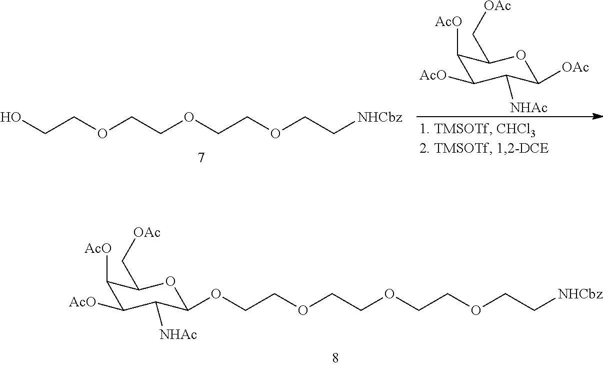 [SMILES: CC(=O)NC1C(OC(C)=O)OC(COC(C)=O)C(OC(C)=O)C1OC(C)=O ; O=C(NCCOCCOCCOCCO)OCc1ccccc1 ; CC(=O)NC1C(OCCOCCOCCOCCNC(=O)OCc2ccccc2)OC(COC(C)=O)C(OC(C)=O)C1OC(C)=O]

To a suspension of commercially available GalNAc acetate (10 g, 25.68 mmol, 1 eq) in 1,2-dichloroethane (50 mL) at rt was added TMSOTf (6.5 mL, 35.96 mmol, 1.4 eq) over 3 min. The mixture was then warmed up to 50° C. and stirred at 50° C. for 5 hr. Compound 7 (11.8 g, 35.957 mmol, 1.4 eq), 4 A molecular sieves (10 g) were added, followed by addition of TMSOTf (1.2 ml, 6.42 mmol, 0.25 eq). The mixture was stirred at rt for 2 days. Filtration, filtrate was washed with brine (100 mL) and Sat NaHCO₃ (50 mL), concentrated, and the residue was purified by flash column chromatography.

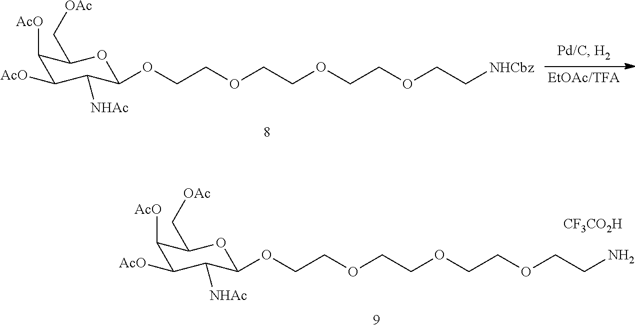 [SMILES: CC(=O)NC1C(OCCOCCOCCOCCNC(=O)OCc2ccccc2)OC(COC(C)=O)C(OC(C)=O)C1OC(C)=O ; CC(=O)NC1C(OCCOCCOCCOCCN)OC(COC(C)=O)C(OC(C)=O)C1OC(C)=O ; "CF3CO2H"]

To a solution of GalNAc-PEG4-NHCbz 8 (8.5 g, 12.9 mmol, 1 eq) in EtOAc (200 ml) and TFA (3 ml) was added Pd/C (1.38 g, 10 wt %, 1.3 mmol, 0.1 eq). The suspension was stirred under H₂ (1 atm) overnight. Then the reaction mixture was filtered through celite and concentrated. The residue was purified by silica-gel column chromatography using a gradient 0-20% MeOH in DCM to afford GalNAc-PEG4-NH₂·TFA 9 (5.90 g, 74%) as a sticky solid.

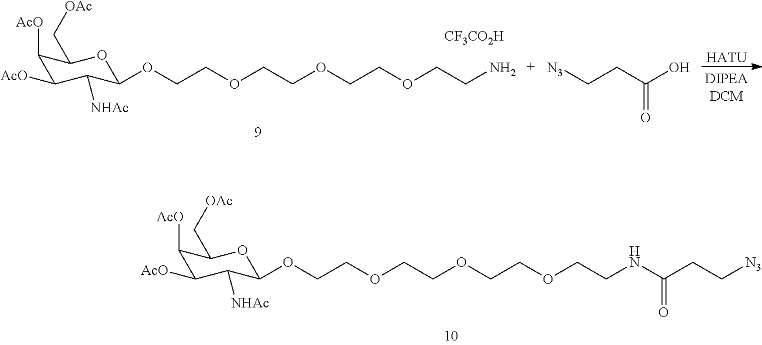

To a solution of GalNAc-PEG4-NH₂·TFA 9 (6.0 g, 9.6 mmol, 1 eq) and 3-azidoacetic acid (1.6 g, 14.5 mmol, 1.5 eq) in DCM (100 ml) at 0° C. was added HATU (5.5 g, 14.5 mmol, 1.5 eq) followed by DIEA (16.9 ml, 96.8 mmol, 10 eq). The reaction was stirred at 0° C. for 5 min, then warmed to room temperature and stirred for 4 h. The reaction was quenched by addition of H₂O. The crude reaction mixture was partitioned between DCM and 1 N HCl. The aqueous phase was extracted with DCM. The combined organic layers were dried over Na₂SO₄, filtered, then concentrated. The residue was purified by silica-gel column chromatography using a gradient 0-10% MeOH in EtOAc to afford the desired product 10 (4.95 g, 83%) as an oil. LCMS ESI+ m/z=620 [M+H]⁺.

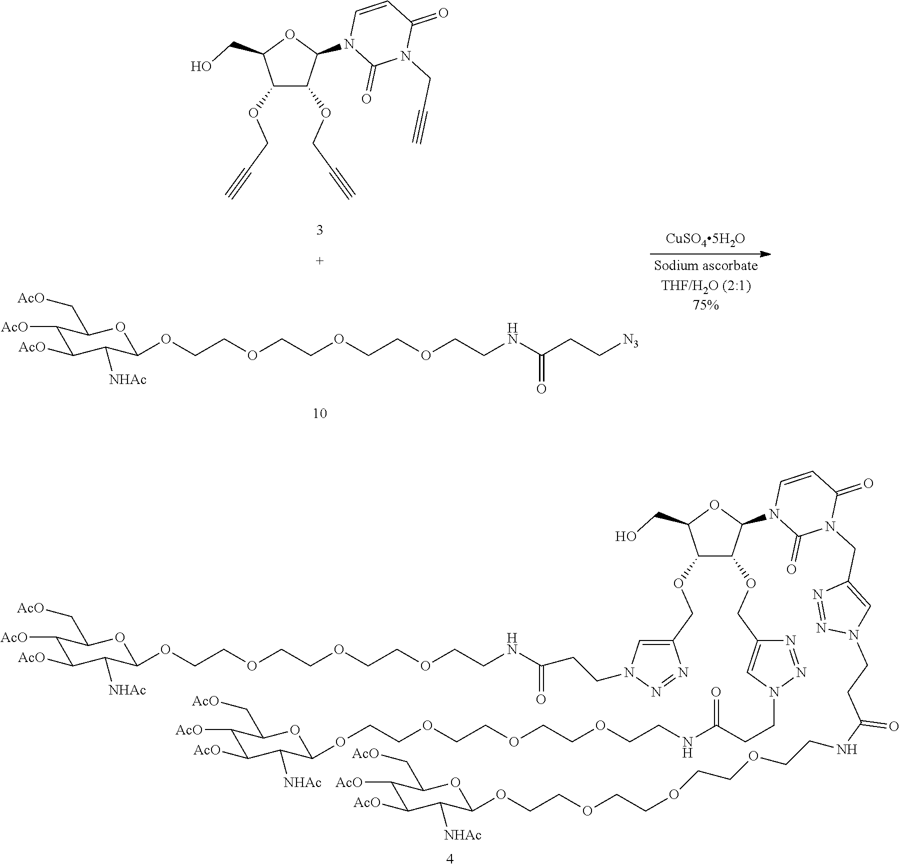 [SMILES: C#CCO[C@@H]1[C@H](OCC#C)[C@@H](CO)O[C@H]1n1ccc(=O)n(CC#C)c1=O ; CC(=O)NC1C(OCCOCCOCCOCCNC(=O)CCN=[N+]=[N-])OC(COC(C)=O)C(OC(C)=O)C1OC(C)=O ; CC(=O)NC1C(OCCOCCOCCOCCNC(=O)CCn2cc(CO[C@@H]3[C@H](OCc4cn(CCC(=O)NCCOCCOCCOCCOC5OC(COC(C)=O)C(OC(C)=O)C(OC(C)=O)C5NC(C)=O)nn4)[C@@H](CO)O[C@H]3n3ccc(=O)n(Cc4cn(CCC(=O)NCCOCCOCCOCCOC5OC(COC(C)=O)C(OC(C)=O)C(OC(C)=O)C5NC(C)=O)nn4)c3=O)nn2)OC(COC(C)=O)C(OC(C)=O)C1OC(C)=O]

To a stirred and cooled 0° C. solution of alkyne 3 (585 mg, 1.634 mmol, 1 eq) and azide 10 (3.136 g, 5.066 mmol, 3.1 eq), in THF (40 mL), were added CuSO₄·5H₂O (0.408 g, 1.634 mmol, 1 eq), in water (10 mL) followed by sodium ascorbate (0.485 g, 2.451 mmol, 1.5 eq) in water (10 mL). The mixture was stirred for 1 h at room temperature. LCMS showed product formation and complete disappearance of alkyne. The reaction mixture was diluted with aq. Saturated NaHCO₃ (100 mL), extracted with DCM 2×200 mL, and washed with brine solution (100 mL). Combined extracts were dried over Na₂SO₄, and concentrated, and the resulting crude was purified by column chromatography using 0-30% MeOH/DCM as an eluent. Pure fractions were combined and concentrated, and the resulting solids were co-evaporated with toluene and dried under high vacuum to obtain tris-triazole 4 (2.75 g 75%) as a yellow solid. LCMS (m/z 1109 M+/2) and NMR are corresponding with product.

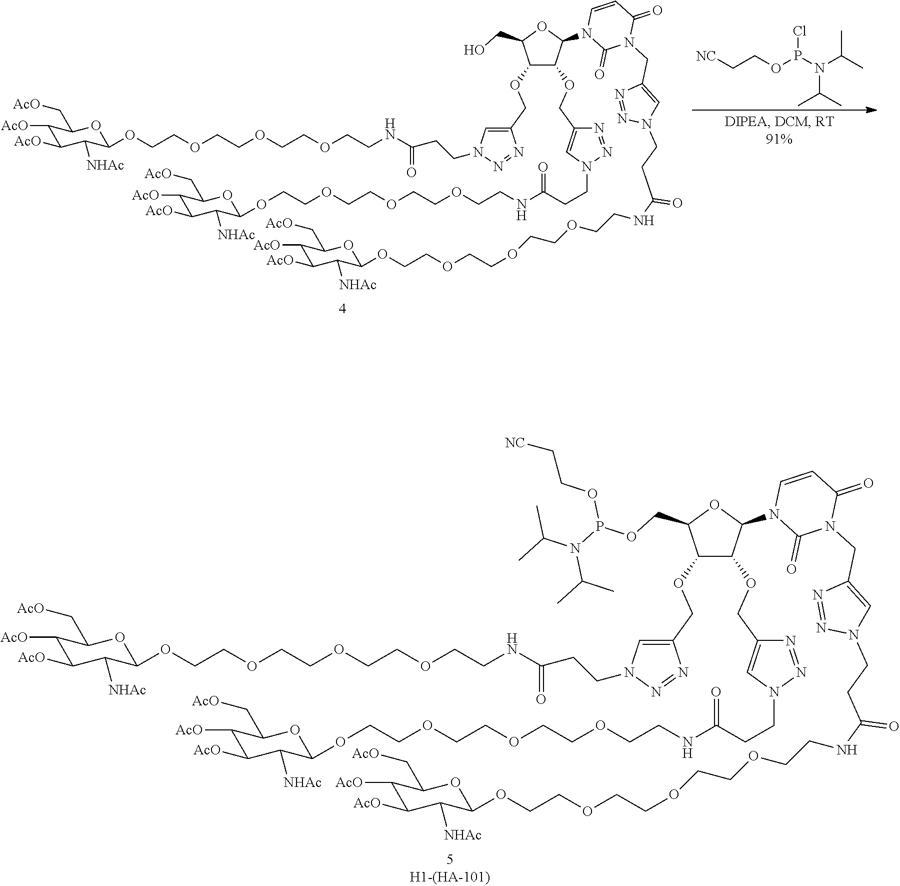

To a stirred solution of alcohol 4 (2.7 g, 1.218 mmol, 1 eq) and diisopropylethylamine (1.37 mL, 7.9 mmol, 6.5 eq), in DCM (20 mL), was added N, N-diisopropyl chlorophosphoramidite (0.8 mL 3.65 mmol, 3 eq) dropwise. The reaction mixture was stirred at room temperature for 15 min. LCMS and HPLC showed completion of reaction. Reaction mixture was quenched with Aq. Saturated NaHCO₃ solution (100 mL), extracted with DCM (2×100) ml, washed with brine (100 mL), dried over N₂SO₄, and the crude product was evaporated and loaded on to pre-equilibrated (2% Et₃N-DCM) Biotage silica gel column (50 g 20 μm). The crude product was purified by flash chromatography using 0-10% MeOH/DCM containing 2% Et₃N as an additive. Pure fractions were combined and concentrated and dried under high vacuum to obtain Phosphoramidite 5 (2.75 g 93%) as a white solid. 91% purity by HPLC, P31-NMR, Mass (m/z 2439 M+Na), H1-NMR and LCMS, HPLC correspond with product.

### Example 8

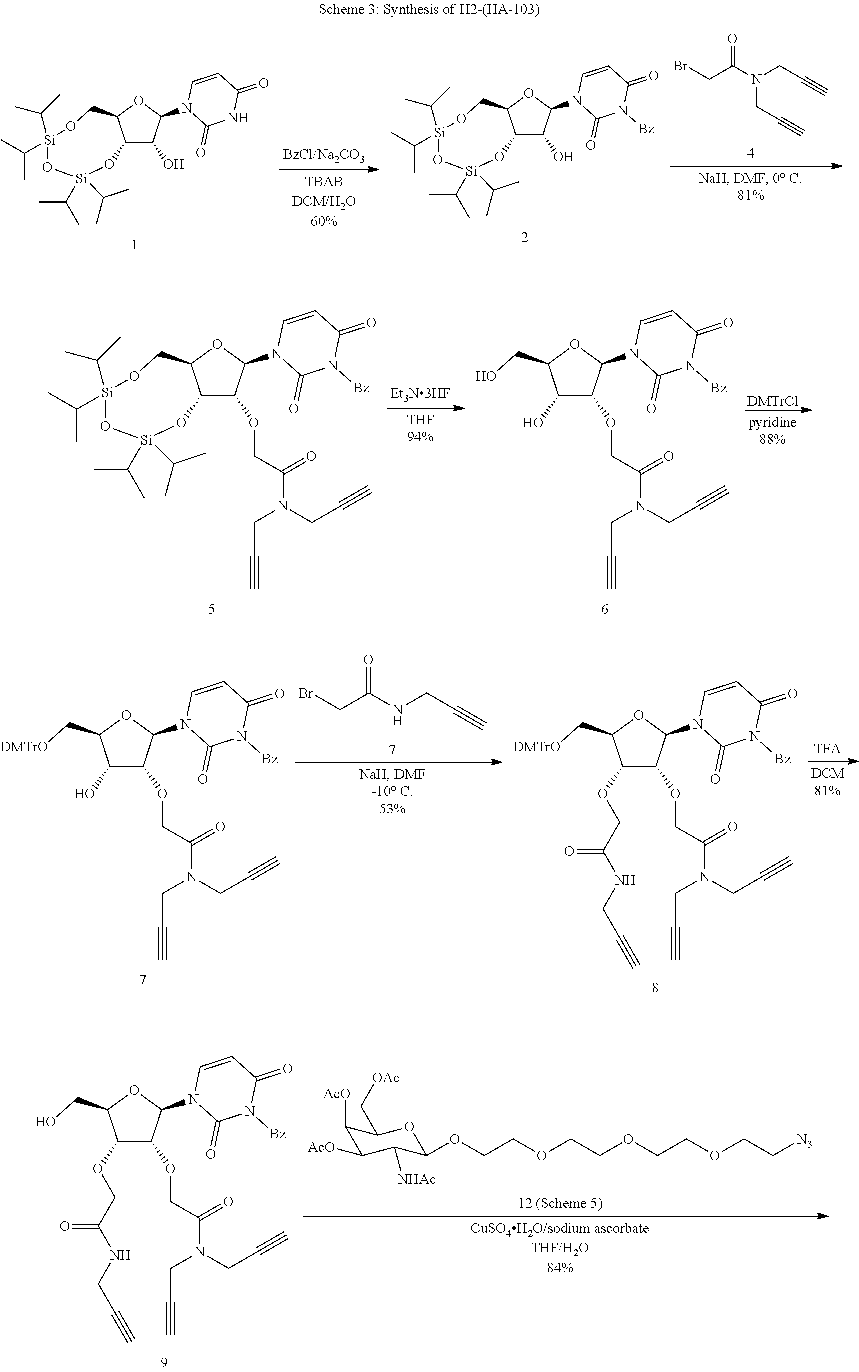

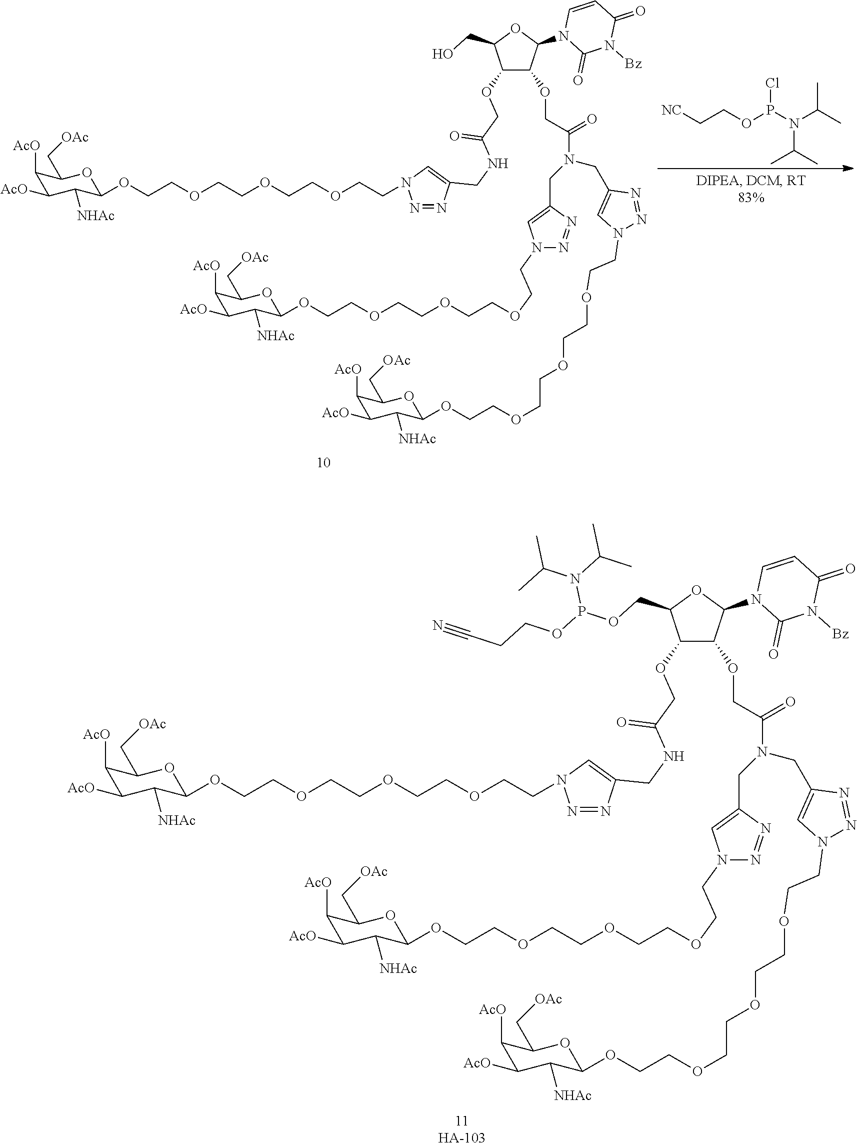

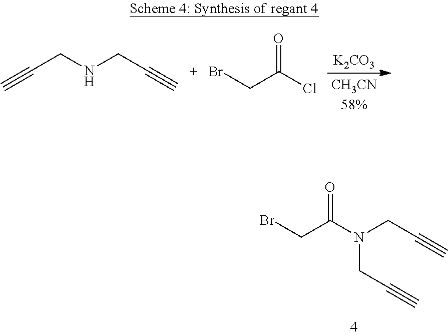

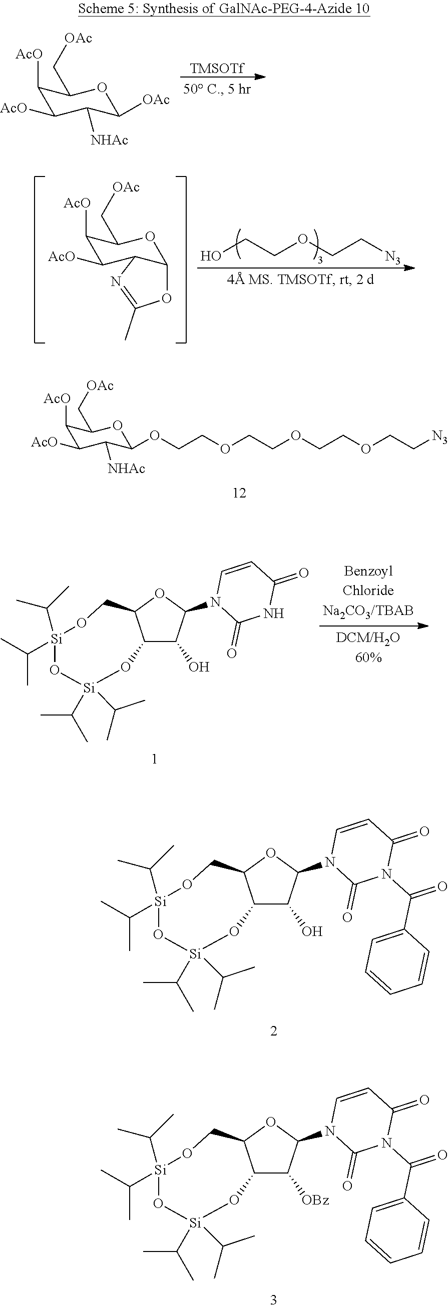

Alcohol 1 (10.0 g, 20.54 mmol, 1 eq), Na₂CO₃ (15.239 g, 143.776 mmol, 7 eq) and tetrabutylammonium bromide (0.265 g, 0.822 mmol, 0.04 eq) were stirred in a biphasic mixture of CH₂Cl₂ (200 mL) and H₂O (400 mL). Benzoyl chloride (3.099 mL, 26.71 mmol, 1.3 eq) was added to the reaction and the reaction mixture was vigorously stirred for 6 hours. The reaction mixture was diluted with CH₂Cl₂. The aqueous phase was separated and extracted with DCM. The combined organic layers were dried over anhydrous Na₂SO₄, filtered, and evaporated under reduced pressure. The residue was dissolved in 1,2-dichloroethane (50 mL) and heated at 60° C. for 20 min. Then the solution was stirred at RT overnight. The solvent was removed under reduced pressure and the residue was purified by column chromatography on silica gel with 0-30% EtOAc in Hexanes to afford 7.3 g (60%) product 2 as a white solid. Compound 3 is the major side product.

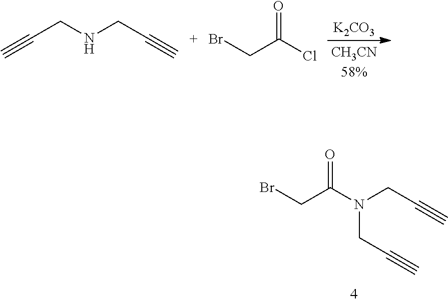

To a suspension di-propargylamine (5 g, 53.688 mmol, 1 eq) and K₂CO₃ (37.1 g, 268.44 mmol, 5 eq) in CH₃CN (100 mL) at 0° C. was added bromoacetyl chloride (4.471 mL, 53.688 mmol, 1 eq). The reaction was stirred for 1 h at 0° C. LCMS showed the reaction was complete. The reaction was diluted with DCM and washed with water. The organic phase was dried by Na₂SO₄, concentrated, and purified by CC (0%→30% EtOAc-hexanes) to afford 6.7 g product 4 (58%) as a yellow oil.

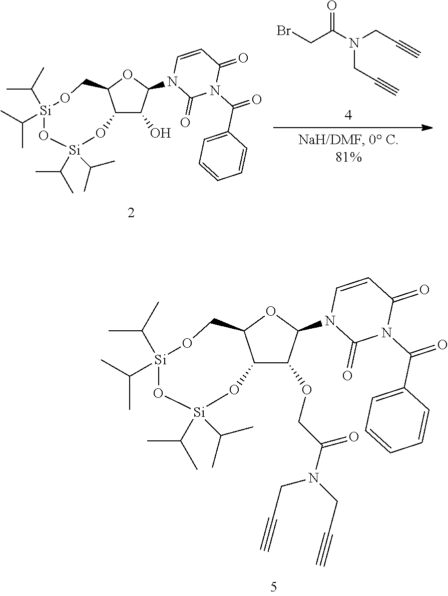

To a stirred solution of alcohol 2 (7.1 g, 12.017 mmol, 1 eq) in DMF (25 mL) at 0° C. was added amide 4 (3.86 g, 18.026 mmol, 1.5 eq) followed by NaH (0.625 g, 60% in mineral oil, 15.622 mmol, 1.3 eq) in two portions. The reaction was stirred at 0° C. for 45 min, then quenched by addition of H₂O. The Reaction mixture was diluted with EtOAc and washed with water. The aqueous phase was extracted with EtOAc. The combined organic layers were dried over Na₂SO₄, filtered, and concentrated. The residue was purified by flash chromatography using 0 to 40% EtOAc in Hexanes to give 7.1 g (81%) product 5 as a white solid.

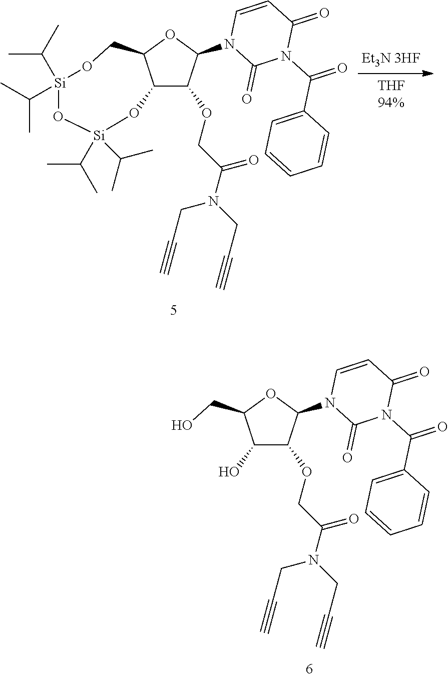

To a stirred solution of bis-alkyne 5 (7.2 g, 9.945 mmol, 1 eq) in THF (100 mL) was added Et₃N·3HF (6.484 mL, 39.781 mmol, 4 eq). The reaction was stirred overnight at RT, and then quenched by addition of MeOH. The Reaction mixture was diluted with EtOAc and washed with NaHCO₃ and brine. The organic layer was dried over Na₂SO₄, filtered, and concentrated. The residue was purified by flash chromatography using 0 to 5% MeOH in EtOAc to give 4.5 g product 6 (94%) as a white solid.

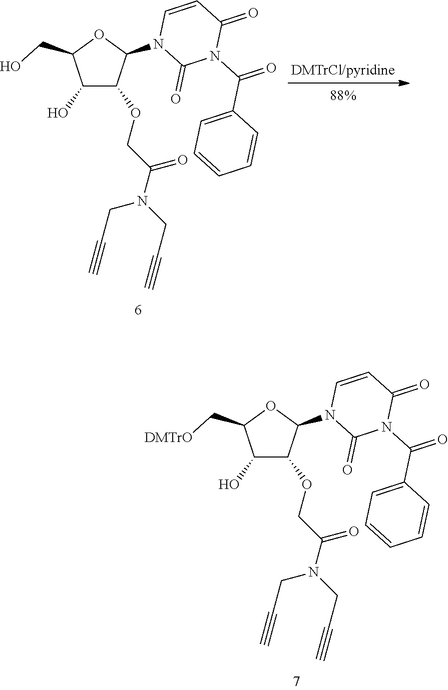

Alcohol 6 was dried by azeotrope distillation with toluene (1×50 mL). To a stirred solution of the alcohol 6 (6.4 g, 13.293 mmol, 1 eq) in anhydrous pyridine (50 mL) was added DMTrCl (5.855 g, 17.281 mmol, 1.3 eq). The reaction was stirred at rt overnight. MeOH was added to quench the reaction. The crude reaction was concentrated under reduced pressure. The residue was partitioned between EtOAc and NaHCO3. The organic layer was dried over Na2SO4, filtered and concentrated. The residue was purified twice by 0-50% EtOAc in hexanes to afforded 9.8 g product 7 (88%) as a white solid.

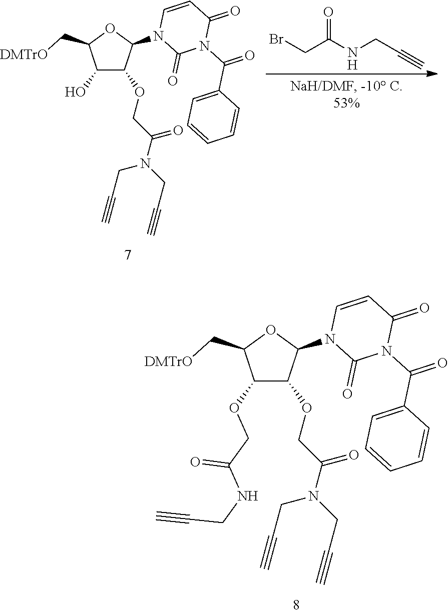

To a stirred solution of DMTr ether 7 (6.0 g, 7.655 mmol, 1 eq) in DMF (40 mL) at −10° C. was added NaH (459 mg, 60% in mineral oil, 11.482 mmol, 1.5 eq) followed by immediate addition of 2-bromo-N-(prop-2-yn-1-yl)acetamide (2.695 g, 15.309 mmol, 2 eq; Synthonix) in DMF (5 mL). The reaction was stirred at −10° C. for 30 min. Then the reaction was quenched by addition of H₂O. The reaction mixture was diluted with EtOAc and washed with water. The aqueous phase was extracted with EtOAc (2×). The combined organic layers were dried over Na₂SO₄, filtered, and concentrated. The residue was purified by flash chromatography using 0 to 60% EtOAc in hexanes to give 3.56 g (53%) of product 8 as a white solid.

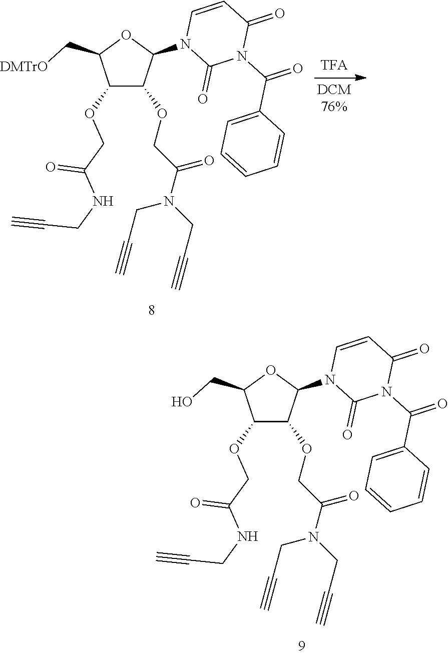

To a solution of DMT ether 8 (3.56 g, 4.05 mmol, 1.00 eq) in DCM (50 mL) at RT was added TFA (0.93 mL, 12.15 mmol, 3.0 eq). The reaction was stirred at RT for 90 min. Additional TFA (0.31 mL, 4.05 mmol, 1.0 eq) was added to the reaction, and the reaction was stirred for 30 min at RT. The reaction was quenched by carefully addition of aq. NaHCO₃. The reaction mixture was partitioned between Saturated NaHCO₃ and DCM. The aqueous phase was extracted with DCM (1×). The organic layers were combined, dried over Na₂SO₄, filtered, and concentrated. The residue was purified by column chromatography on silica gel using 0-10% MeOH in DCM to give 1.89 g product 9 (81%) as a white solid.

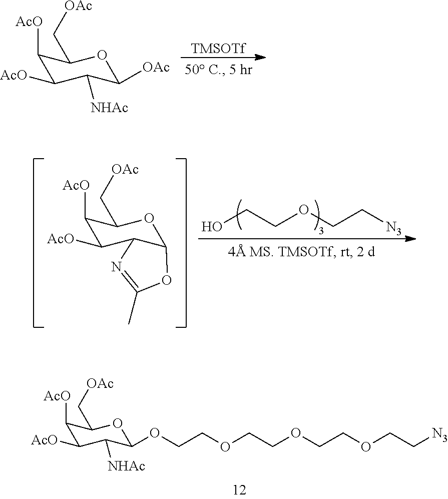

To a suspension of commercially available GalNAc-Acetate (64 g, 164.20 mmol, 1.2 eq) in 1,2-dichloroethane (300 mL) at rt was added TMSOTf (29.7 mL, 164.21 mmol, 1.2 eq, d:1.228) over 5 min, then the mixture was warmed up to 50° C. and stirred at 50° C. for 5 hr. The mixture was cooled down to rt, then was added 4 A MS (60 g) followed by commercially available HO-PEG-6-N₃ (30.0 g, 136.84 mmol, 1.0 eq). After stirring at rt for 10 min, TMSOTf (7.4 mL, 41.05 mmol, 0.3 eq) was added. The resultant mixture was stirred at rt for 2 days. Work-up: the reaction mixture was filtrated and washed with DCM (50 mL). Filtrate was poured into brine (100 mL)+Sat NaHCO₃ (150 mL). Org. layer was separated, Aq. Phase was extracted with DCM (50 mL). The combined Org. layers were washed with brine (100 mL)+Sat NaHCO₃ (100 mL), and concentrated. The residue was purified by flash chromatography, using 50% EtOAc/Hept to EtOAc to 1%-6% MeOH/EtOAc. To obtain azide 12 37.2 g (60%) as a pale-yellow syrup.

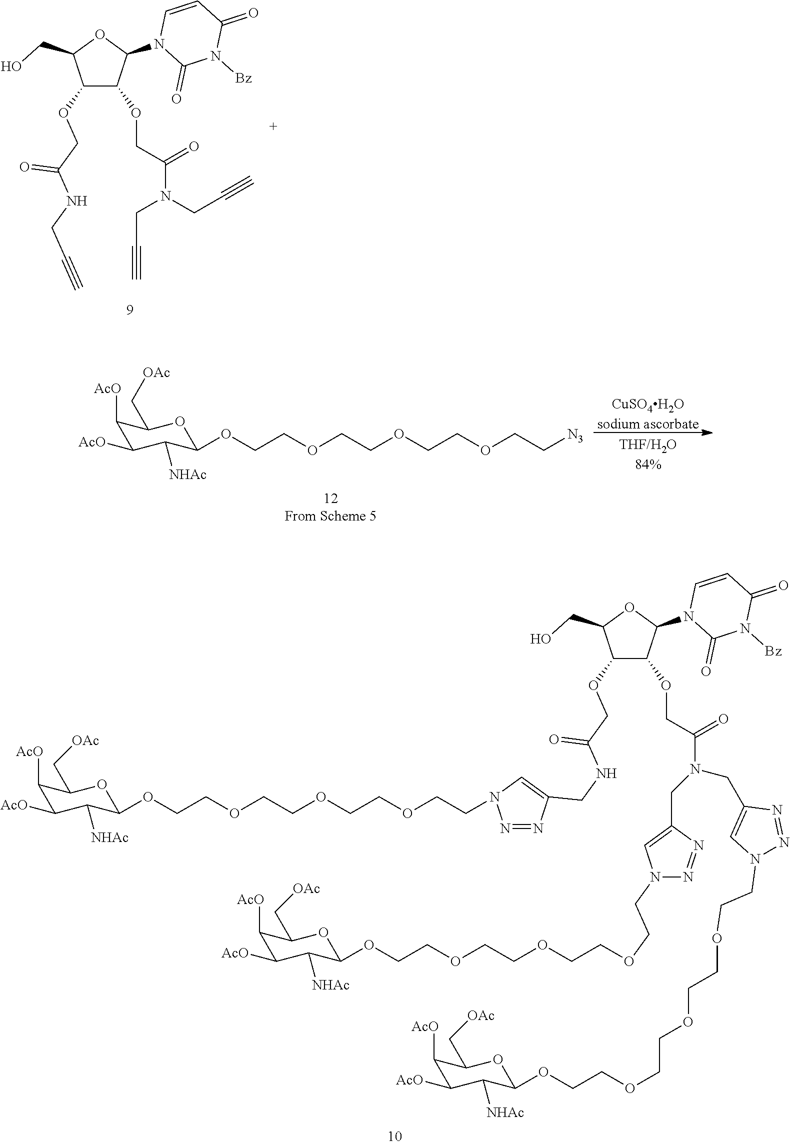

To a solution of alcohol 9 (2.0 g, 3.469 mmol, 1 eq) and azide 12 (6.089 g, 11.1 mmol, 3.2 eq) in THF (30 mL) at 0° C. was added a solution of CuSO₄·5H₂O (0.260 g, 1.041 mmol, 0.3 eq) in water (7.5 mL) and a solution of sodium ascorbate (0.309 g, 1.561 mmol, 0.45 eq) in water (7.5 mL). After stirring for 5 min at 0° C., the reaction was warmed to RT. The reaction was stirred for 3 hours at RT. LCMS showed the reaction was completed. The reaction was diluted with DCM and washed with NaHCO₃. The aqueous phase was extracted with DCM (4×). The organic layers were combined, dried over Na₂SO₄, filtered, and concentrated. The residue was purified by 0-20% MeOH in DCM to give 6.46 g triazole 10 (84%) as a yellow solid.

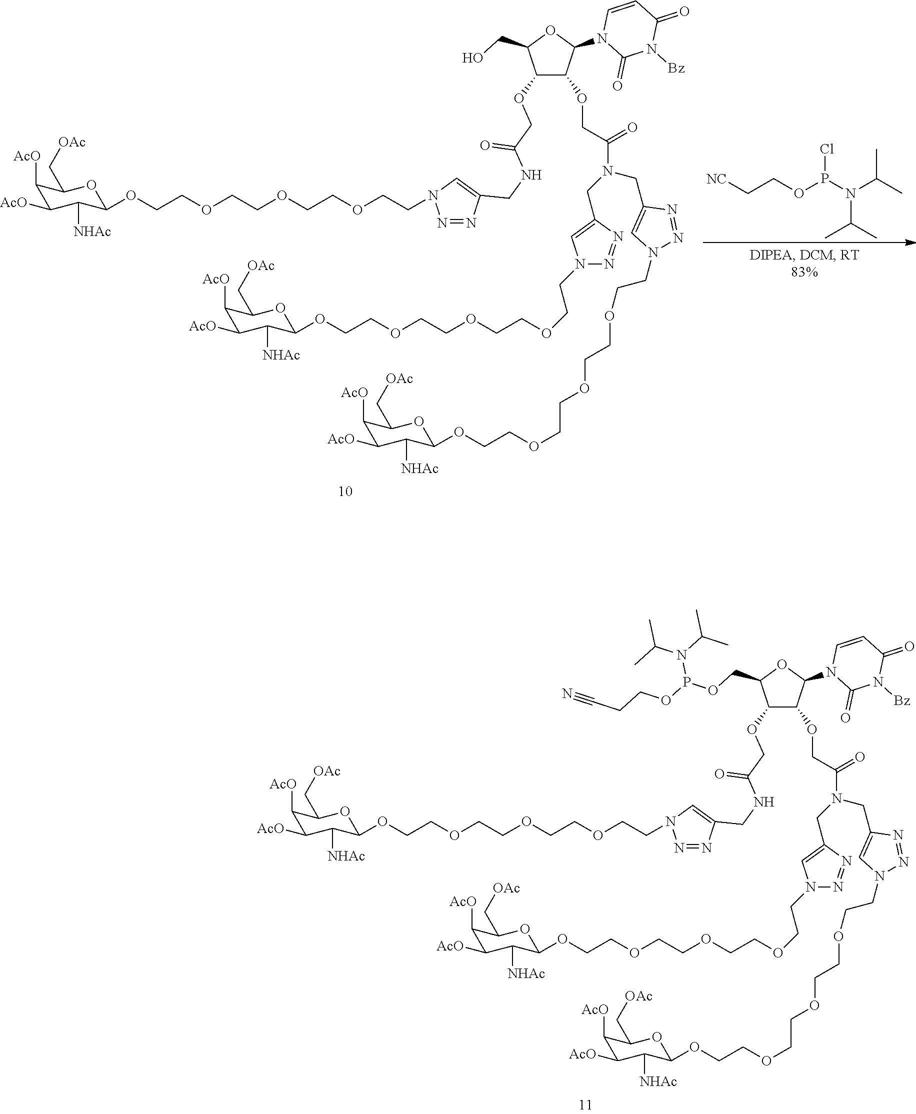

To a stirred solution of triazole 10 (6.1 g, 2.745 mmol, 1 eq) and diisopropylethylamine (3.108 mL, 17.843 mmol, 6.5 eq) in anhydrous DCM (100 mL) was added 2-cyanoethyl N,N-diisopropyl chlorophosphoramidite (1.837 mL, 8.235 mmol, 3 eq) dropwise. The reaction mixture was stirred at room temperature for 45 min. LCMS and HPLC showed completion of reaction. The reaction mixture was quenched with saturated NaHCO₃ solution and partitioned between DCM and saturated NaHCO₃. The DCM phase was collected. The aqueous phase was extracted with DCM (1×). The combined organic phases were washed with brine, dried over Na₂SO₄, filtered, and concentrated. The residue was loaded on to a pre-equilibrated (2% Et₃N-DCM) biotage silica gel column (50 g, 20 μm) and purified by flash chromatography using 0-5-10% MeOH/DCM containing 2% Et₃N as an additive. Pure fractions were combined, concentrated, and dried under high vacuum to obtain 5.51 g phosphoramidite 11 (83%) (97% purity HPLC).

### Example 9

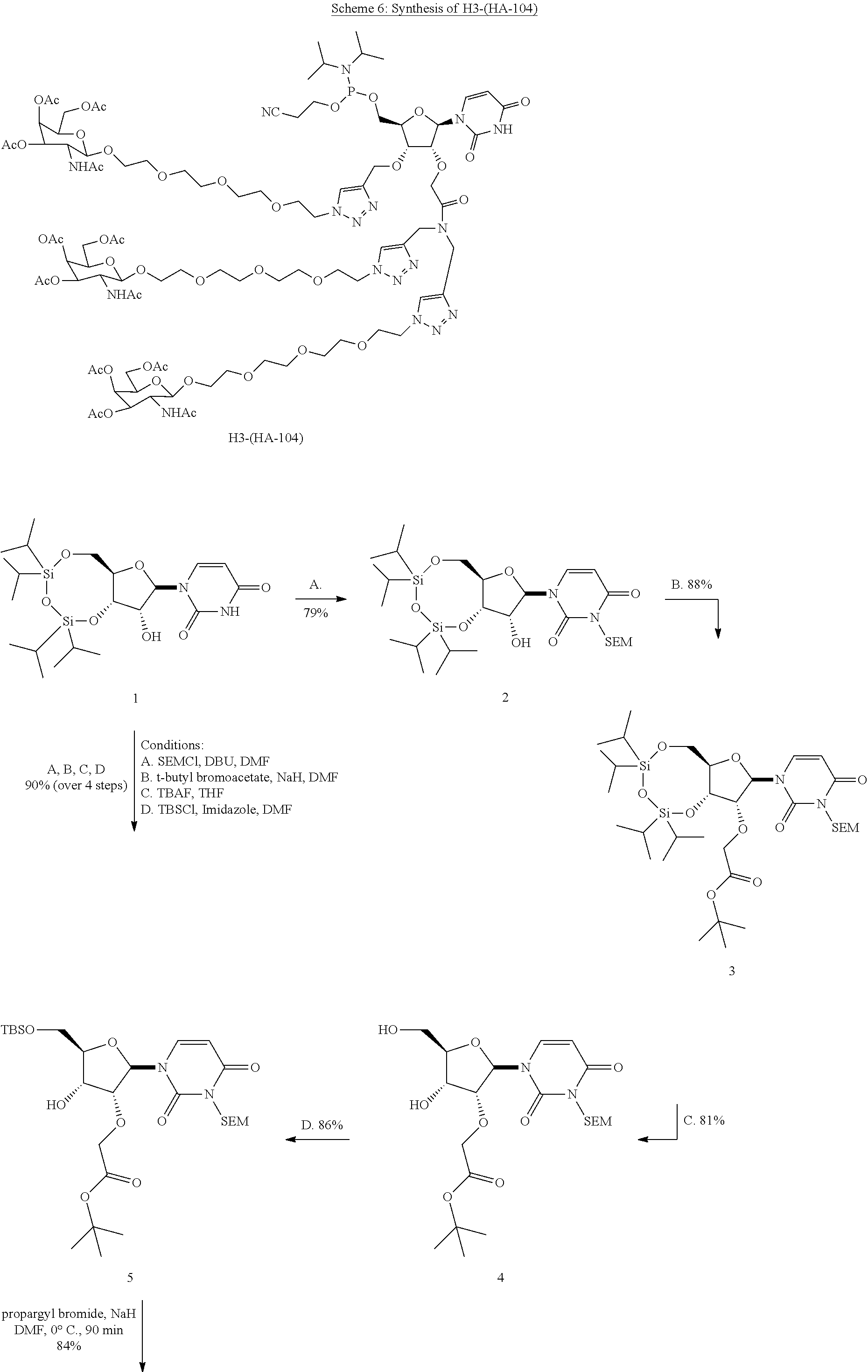

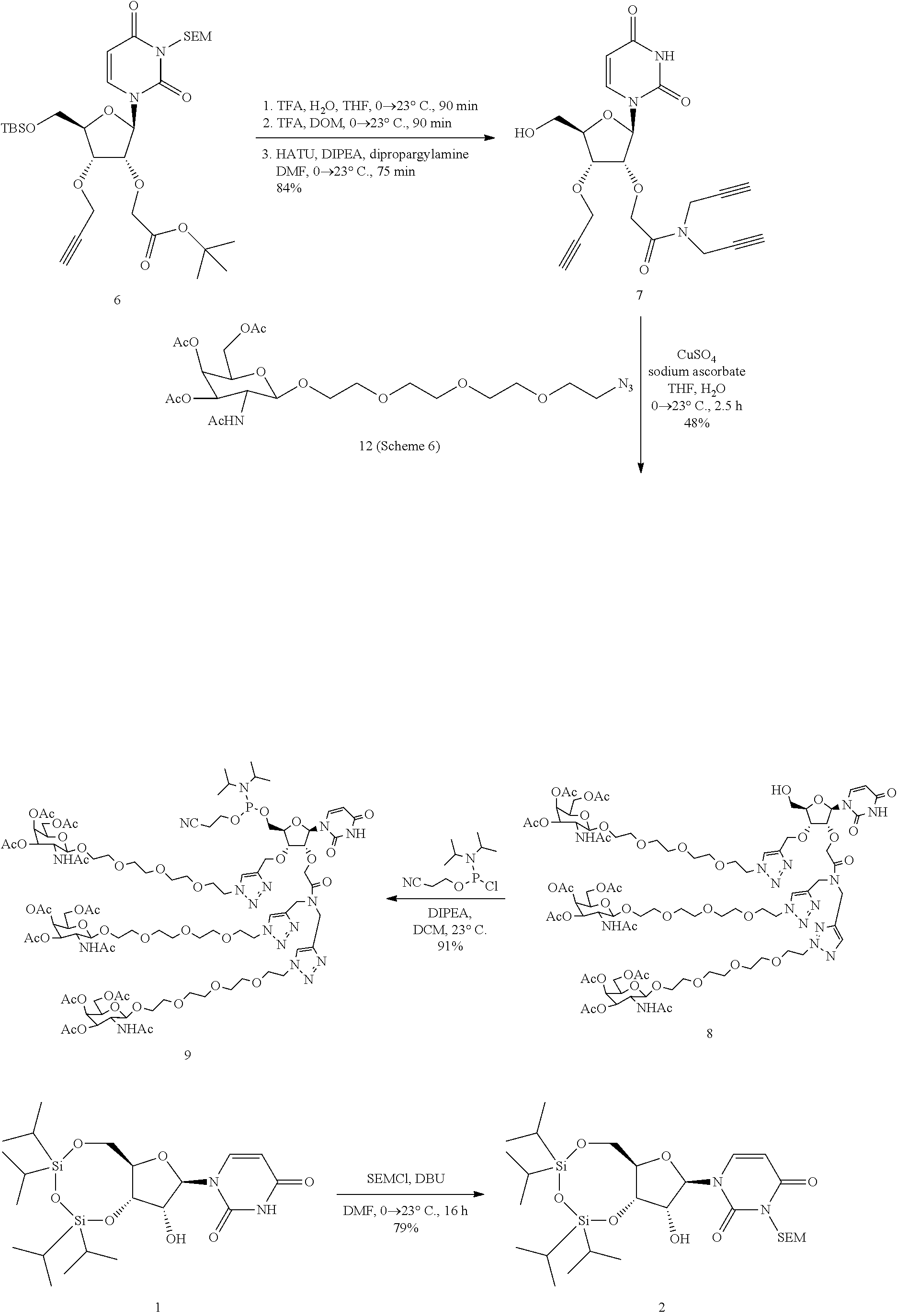

SEMCl (3.3 mL, 18.5 mmol, 1.2 equiv) was added dropwise to a solution of alcohol 1 (7.5 g, 15.4 mmol, 1 equiv) and DBU (3.5 mL, 23 mmol, 1.5 equiv) in DMF (50 mL) at 0° C. The reaction mixture was left in the ice-water bath to slowly warm to room temperature overnight. After 16 h, the reaction mixture was diluted with ethyl-acetate hexanes (5:1, 350 mL) and poured into cold stirring water (500 mL). The organic layer was washed with water (2×300 mL), and with a saturated aqueous sodium chloride solution (200 mL). The aqueous layers were separately extracted with a single portion of ethyl acetate-hexanes (5:1, 250 mL). The combined organic extracts were dried over anhydrous sodium sulfate, filtered, and concentrated under reduced pressure. The resulting colorless oil (10 g) was purified by flash column chromatography on silica gel (50 g, gradient: ethyl acetate in hexanes, 0→15% [3CV], 15% [3CV], 15→20% [4CV], 20% [2CV], 20→25% [3CV], 25% [4CV]) to afford alcohol 2 as a colorless oil (7.5 g, 79%). TLC (25% ethyl acetate in hexanes). LCMS: ESI+ m/z 639.45 [M+Na]⁺.

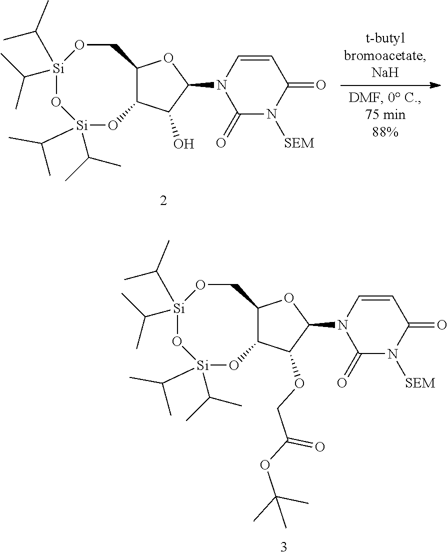 [SMILES: CC(C)[Si]1(C(C)C)OC[C@H]2O[C@@H](n3ccc(=O)n(COCC[Si](C)(C)C)c3=O)[C@H](O)[C@@H]2O[Si](C(C)C)(C(C)C)O1 ; CC(C)[Si]1(C(C)C)OC[C@H]2O[C@@H](n3ccc(=O)n(COCC[Si](C)(C)C)c3=O)[C@H](OCC(=O)OC(C)(C)C)[C@@H]2O[Si](C(C)C)(C(C)C)O1]

Alcohol 2 (7.5 g, 12.2 mmol, 1 equiv) was dried azeotropically by concentration from anhydrous toluene (20 mL) under reduced pressure at 35° C. The resulting oil was dissolved in DMF (50 mL) and cooled to 0° C. t-butyl bromoacetate (4.5 mL, 26 mol, 2.5 equiv) was added, followed by sodium hydride (60% dispersion in mineral oil, 608 mg, 15.2 mmol, 1.25 equiv). The reaction mixture was maintained at 0° C. After 70 min, additional sodium hydride (230 mg, 5.8 mmol, 0.5 equiv) was added as a solid in a single portion. After 2.5 h total reaction time, the reaction mixture was quenched with methanol (2 mL) and was then immediately poured into stirring ethyl acetate-hexanes (4:1, 250 mL) and 25% saturated aqueous sodium chloride solution (500 mL). The organic layer was washed with 10% saturated aqueous sodium chloride solution (2×250 mL), with water (100 mL), and with a saturated aqueous sodium chloride solution (100 mL). The aqueous layers were extracted with a single portion of ethyl acetate-hexanes (4:1, 250 mL). The combined organic layers were dried over anhydrous sodium sulfate, filtered, and concentrated under reduced pressure. The resulting colorless residue (12.2 g) was purified by flash column chromatography on silica gel (eluent: ethyl acetate in hexanes, gradient: 0% [2CV], 0→5% [2CV], 5% [1CV], 5→10% [5CV], 10% [1CV], 10→15% [3 CV, minor elutes, then major], 15% [1CV], 15→20% [1CV], 20% [5CV]) to afford ester 3 (7.8 g, 88%) as a colorless oil. TLC (20% EtOAc/Hexanes). LCMS: ESI+ m/z 753.41 [M+Na]⁺.

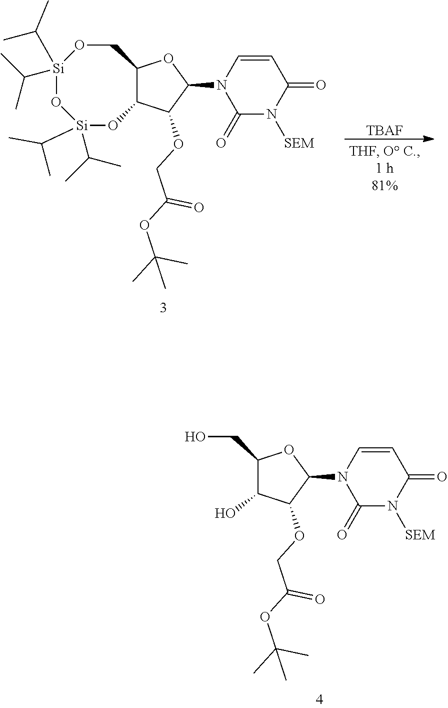

TBAF (1.0 M in THF, 30 mmol, 2.8 equiv) was added dropwise to a solution of disiloxane 3 (7.8 g, 10.7 mmol, 1 equiv) in THF (100 mL, 0.1 M) at 0° C. After 6 h at 0° C., the reaction mixture was poured into a saturated aqueous solution of sodium bicarbonate (250 mL) and was extracted with DCM (3×150 mL). The combined organic extracts were washed with water (250 mL) and with a saturated aqueous sodium chloride solution (150 mL). The aqueous layers were separately extracted with a single portion of DCM (150 mL). The combined organic layers were dried over anhydrous sodium sulfate, filtered, and concentrated under reduced pressure. The resulting colorless oil (10 g) was purified by flash column chromatography on silica gel (50 g, gradient: ethyl acetate in hexanes, 0→60% [7CV], 60→75% [2CV], 75% [2CV], 75→85% [2CV], 85% [4 CV]) to afford diol 4 (4.2 g, 81%) as a sticky white foam. LCMS: ESI+ m/z 511.09 [M+Na]⁺.

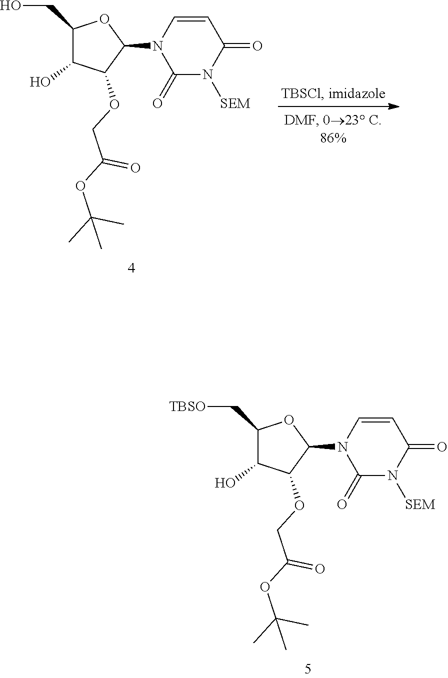 [SMILES: CC(C)(C)OC(=O)CO[C@@H]1[C@H](O)[C@@H](CO)O[C@H]1n1ccc(=O)n(COCC[Si](C)(C)C)c1=O ; CC(C)(C)OC(=O)CO[C@@H]1[C@H](O)[C@@H](CO[Si](C)(C)C(C)(C)C)O[C@H]1n1ccc(=O)n(COCC[Si](C)(C)C)c1=O]

Diol 4 (4.2 g, 8.6 mmol, 1 equiv) was azeotropically dried by concentration from anhydrous DCM (10 mL) and toluene (20 mL) at 35° C. TBSCl (1.56 g, 10.4 mmol, 1.2 equiv) was added as a solid in one portion to a solution of diol 4 and imidazole (1.17 g, 17.2 mmol, 2 equiv) in DMF (40 mL) at 0° C. The reaction mixture was removed from the ice bath and allowed to stir at room temperature. After 20 h, the reaction mixture was diluted with ethyl acetate-hexanes (5:1, 250 mL), and was then poured into stirring cold water (400 mL). The organic layer was washed with water (3×300 mL) and with a saturated aqueous sodium chloride solution (250 mL). The aqueous layers were separately extracted with a single portion of ethyl acetate-hexanes (5:1 150 mL). The organic layer was dried over anhydrous sodium sulfate, filtered, and concentrated under reduced pressure. The resulting crude residue (6.6 g) was purified by flash column chromatography on silica gel (50 g, 60 uM, gradient: ethyl acetate in hexanes, 0→10% [3CV], 10% [6CV], 10→25% [5 CV], 25% [4CV]) to afford alcohol 5. Mixed fractions (1.1 g) were repurified by flash column chromatography on silica gel (25 g, 60 uM, gradient: ethyl acetate in hexanes, 0→5% [3CV], 5% [5CV], 5→10% [8CV], 10% [5CV]) to afford additional alcohol 5, which was combined with earlier pure fractions to afford alcohol 5 (4.43 g, 85.5% yield) as a colorless sticky oil. TLC (25% EtOAc/hexanes). LCMS: ESI+ m/z 625.25 [M+Na]⁺.

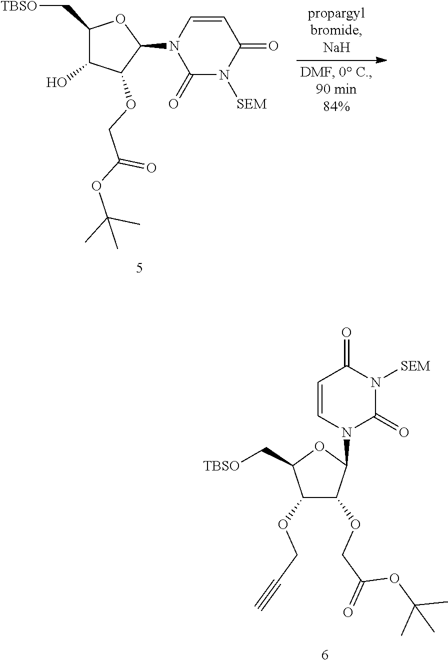 [SMILES: CC(C)(C)OC(=O)CO[C@@H]1[C@H](O)[C@@H](CO[Si](C)(C)C(C)(C)C)O[C@H]1n1ccc(=O)n(COCC[Si](C)(C)C)c1=O ; C#CCO[C@H]1[C@@H](OCC(=O)OC(C)(C)C)[C@H](n2ccc(=O)n(COCC[Si](C)(C)C)c2=O)O[C@@H]1CO[Si](C)(C)C(C)(C)C]

Alcohol 5 (4.43 g, 7.18 mmol, 1 equiv) was azeotropically dried by concentration from anhydrous toluene (10 mL) under reduced pressure. The resulting residue was dissolved in anhydrous DMF (35 mL, 0.2 M) and cooled to 0° C. Propargyl bromide (80% w/w in toluene, 2.4 mL, 22 mmol, 3 equiv) was added, followed by NaH (431 mg, 10.8 mmol, 1.5 equiv) as a solid in one portion. After 1 h, additional propargyl bromide (80% w/w in toluene, 1.6 mL, 11 mmol, 1.5 equiv) was added, followed by NaH (144 mg, 3.6 mmol, 0.5 equiv) as a solid in one portion. After 1 h (2 hrs total reaction time), the reaction mixture was diluted with ethyl acetate-hexanes (5:1, 350 mL) and was washed with 10% saturated brine (3×350 mL), and with a saturated aqueous sodium chloride solution (250 mL). The aqueous layers were separately extracted with a single portion of ethyl acetate-hexanes (5:1, 150 mL). The combined organic layers were dried over anhydrous sodium sulfate, filtered, and concentrated under reduced pressure. The resulting brown oil (5.2 g) was purified by flash column chromatography on silica gel (50 g, 60 μm, eluent: ethyl acetate in hexanes, 0% [2CV], 0→7% [5CV], 7% [4CV], 7→12% [3CV], 12% [2CV], 12→15% [2 CV], 15% [3CV]) to afford alkyne 6 (3.85 g, 83.7%) as a light yellow oil. LCMS: ESI+ m/z 663.36 [M+Na]⁺.

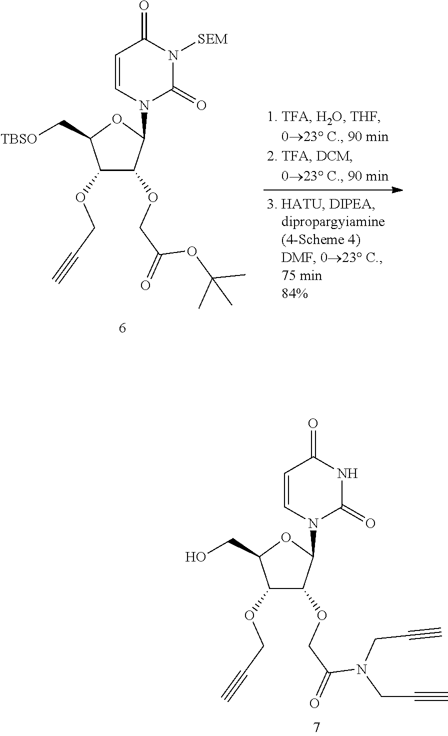 [SMILES: C#CCO[C@H]1[C@@H](OCC(=O)OC(C)(C)C)[C@H](n2ccc(=O)n(COCC[Si](C)(C)C)c2=O)O[C@@H]1CO[Si](C)(C)C(C)(C)C ; C#CCO[C@H]1[C@@H](OCC(=O)N(CC#C)CC#C)[C@H](n2ccc(=O)[nH]c2=O)O[C@@H]1CO]

TFA (2.5 mL) was added dropwise to a solution of alkyne 6 (0.98 g, 1.53 mmol, 1 equiv) in THF (10 mL) and H₂O (2.5 mL) at 0° C. The reaction mixture was removed from the ice-water bath and allowed to stir at room temperature. After 1.5 h, the reaction mixture was concentrated under reduced pressure at 37° C. The resulting residue was dissolved in dichloromethane (10 mL), cooled to 0° C., then TFA (5 mL) was added. After 1.5 h, the reaction mixture was concentrated under reduced pressure at 37° C. The resulting residue was diluted with DCM (2 mL) and toluene (2 mL), then concentrated under reduced pressure. The resulting brown oil was dissolved in DMF (5 mL), cooled to 0° C., then DIPEA (1.3 mL, 7.65 mmol, 5 equiv), dipropargyl amine (0.8 mL, 7.65 mmol, 5 equiv), and HATU (1.098 g, 2.89 mmol, 1.9 equiv) were added consecutively. The reaction mixture was removed from the ice-water bath and allowed to stir at room temperature. After 75 min, the reaction mixture was diluted with ethyl acetate-hexanes (9:1, 50 mL) and with 1 N HCl (50 mL) and was stirred vigorously for 5 minutes. The aqueous layer was extracted with ethyl acetate-hexanes (9:1, 3×50 mL). The combined organic extracts were washed with water (2×50 mL) and with a saturated aqueous sodium chloride solution (50 mL). The aqueous layers were separately extracted with ethyl acetate-hexanes (4:1, 50 mL). The combined organic layers were dried over anhydrous sodium sulfate, filtered, and concentrated under reduced pressure. The resulting yellow solid (1.62 g) was suspended in dichloromethane (˜10 mL) and was filtered through a plastic frit. The filter cake was washed with DCM (2×10 mL), and the combined filtrates were concentrated under reduced pressure. The resulting residue (1 g) was purified by flash column chromatography on silica gel (25 g, gradient: ethyl acetate in hexanes, 0% [2CV] 0→60% [5CV], 60% [2CV], 60→90% [3CV], 90% [4CV], 90→95% [3CV], 95% [4CV]) to afford amide 7 (532 mg, 84%) as an off white-foam. LCMS: ESI+ m/z 438.04 [M+Na]⁺.

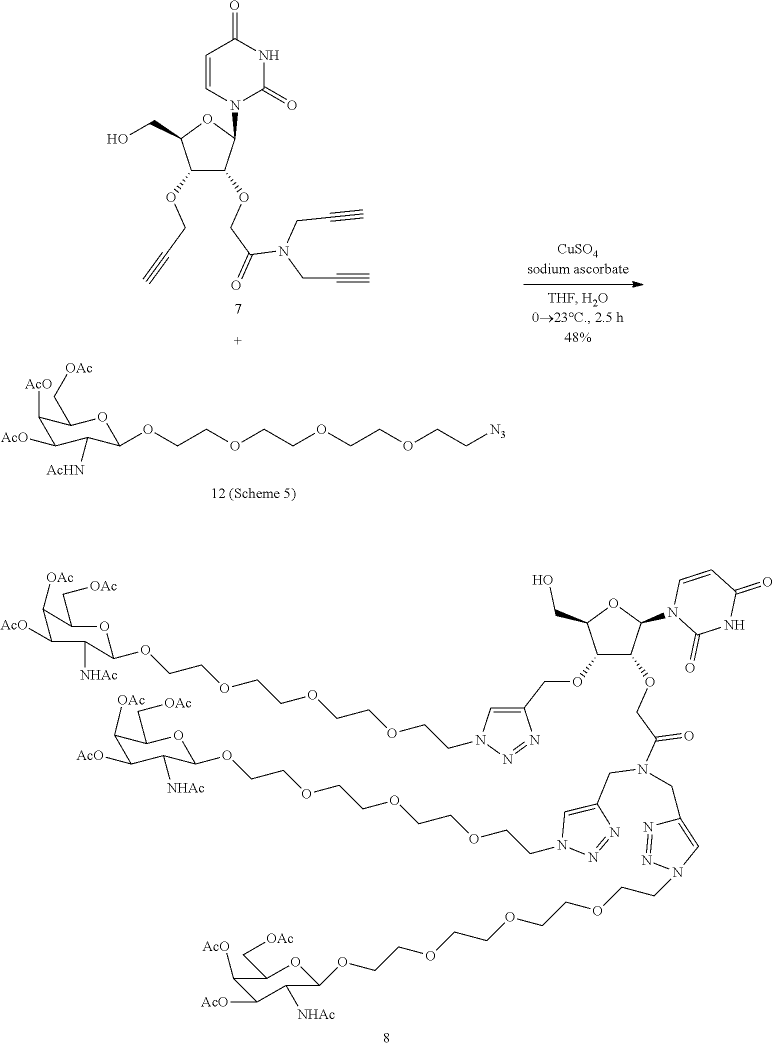 [SMILES: C#CCO[C@H]1[C@@H](OCC(=O)N(CC#C)CC#C)[C@H](n2ccc(=O)[nH]c2=O)O[C@@H]1CO ; CC(=O)NC1C(OCCOCCOCCOCCN=[N+]=[N-])OC(COC(C)=O)C(OC(C)=O)C1OC(C)=O ; CC(=O)NC1C(OCCOCCOCCOCCn2cc(CO[C@H]3[C@@H](OCC(=O)N(Cc4cn(CCOCCOCCOCCOC5OC(COC(C)=O)C(OC(C)=O)C(OC(C)=O)C5NC(C)=O)nn4)Cc4cn(CCOCCOCCOCCOC5OC(COC(C)=O)C(OC(C)=O)C(OC(C)=O)C5NC(C)=O)nn4)[C@H](n4ccc(=O)[nH]c4=O)O[C@@H]3CO)nn2)OC(COC(C)=O)C(OC(C)=O)C1OC(C)=O]

Water and THF for stock solutions and reactions was sparged for 15 min with argon via a needle from a balloon. Tris-alkyne (780 mg, 1.88 mmol, 1 equiv) and azide (3.2 g, 5.9 mmol, 3.2 equiv) were placed under an argon atmosphere, were dissolved in THF (18 mL), and were sparged with argon for 15 min. Solutions of sodium ascorbate and copper sulfate (2× the mass and volume needed for reaction) in water were prepared in scintillation vials and sparged with argon for 15 min. A solution of sodium ascorbate (151 mg, 0.76 mmol, 0.4 equiv) in water (4.5 mL) was added, then a solution of copper sulfate pentahydrate (94 mg, 0.38 mmol, 0.2 equiv) in water (4.5 mL) was added dropwise over 5 min to the solution of alkyne and azide in THF at 0° C. After 5 min, the reaction-mixture was removed from the ice-water bath and allowed to warm to room temperature. After 2.5 h, the reaction mixture was concentrated under reduced pressure (50 torr) at 35° C. to remove THF. The reaction mixture was diluted with DCM (100 mL) and washed with 50% saturated aqueous sodium bicarbonate solution (100 mL), with water (100 mL), and with a saturated aqueous sodium chloride solution (100 mL). The aqueous layers were separately extracted with dichloromethane (2×50 mL). The combined organic extracts were dried over anhydrous sodium sulfate, filtered, and concentrated under reduced pressure. The resulting off-white foam (2.8 g total) was repurified by flash column chromatography on silica gel (25 g, High Capacity 20 μm cartridge, gradient: methanol in dichloromethane, 0% [2CV], 0→10% [4CV], 10→15% [8CV], 15% [13CV], 15→20% [5 CV], 20% [10CV]) to afford clicked product 8 (1.86 g, 96% HPLC purity, 48% yield) and lower purity batch (494 mg, 90% purity, 12% yield). LCMS: ESI+ m/z 2082.8 [M+Na]⁺.

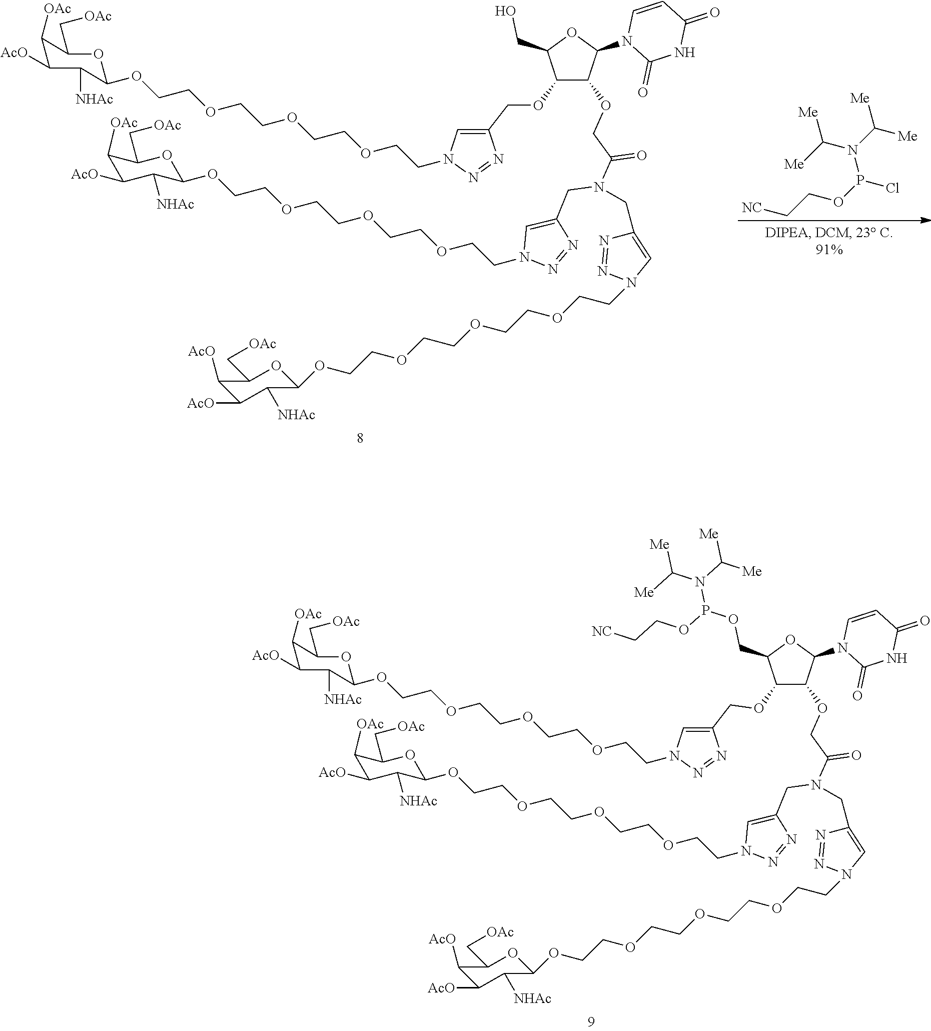

Alcohol 8 (1.86 g, 0.903 mmol, 1 equiv) was azeotropically dried by concentration from anhydrous DCM (10 mL) and toluene (15 mL) at 35° C. under reduced pressure on the rotavap, with backfilling of argon via the Schlenk port of the flask. DCM for the reaction was sparged in bulk with argon for 15 minutes. Alcohol 8 was dissolved in anhydrous DCM (30 mL), then DIPEA (1.1 mL, 6.3 mmol, 7 equiv) was added, followed by 2-cyanoethyl N,N-diisopropylchloro-phosphoramidite (0.60 mL, 2.7 mmol, 3 equiv) dropwise. HPLC analysis at 10 minutes showed full conversion. After 25 min, the reaction mixture was quenched by the direct addition of 50% saturated aqueous sodium bicarbonate solution (50 mL). The aqueous layer was extracted with DCM (2×50 mL). The combined organic extract was washed with a 10% saturated aqueous sodium bicarbonate solution (50 mL—a slight emulsion forms, add 20 mL saturated sodium chloride solution to help break), and with a saturated aqueous sodium chloride solution (50 mL). The aqueous layers were separately extracted with DCM (2×50 mL). The combined organic extracts were dried over anhydrous sodium sulfate, filtered, and concentrated under reduced pressure. The resulting white foam (3.2 g) was purified by flash column chromatography on silica gel (25 g high capacity 20 μm column, pre-equilibrated with 3 CV of 2% triethylamine in dichloromethane, gradient: 2% triethylamine constant, methanol in dichloromethane, 0% [2CV], 0→4% [8CV], 4→5% [5CV], 5→7% [5CV], 7→10% [5CV], 10% [2CV]) to afford amidite. Fractions were analyzed by HPLC for purity: f14 (96%), f15 (94.5%), f16 (94%), f17 (91%), f18 (88%), f19 (86%), f22 (34%). Fractions f14-17 were combined to afford amidite 9 (2.00 g, 93% HPLC purity→1.86 g, 91% yield) as a white foam (1H and 31P NMR are clean -DMSO-d6).

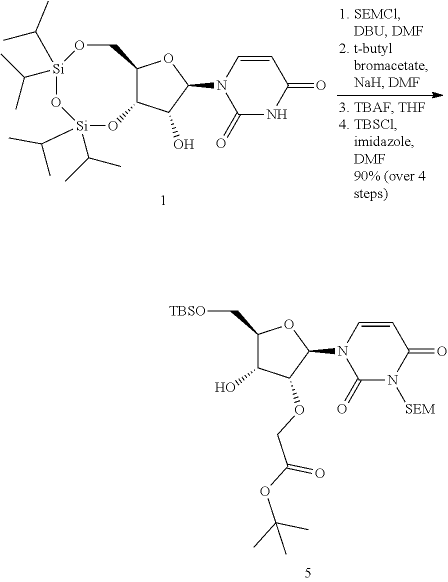 [SMILES: CC(C)[Si]1(C(C)C)OC[C@H]2O[C@@H](n3ccc(=O)[nH]c3=O)[C@H](O)[C@@H]2O[Si](C(C)C)(C(C)C)O1 ; CC(C)(C)OC(=O)CO[C@@H]1[C@H](O)[C@@H](CO[Si](C)(C)C(C)(C)C)O[C@H]1n1ccc(=O)n(COCC[Si](C)(C)C)c1=O]

STEP 1: SEMCl (0.87 mL, 4.9 mmol, 1.2 equiv) was added dropwise to a solution of disiloxane (2.00 g, 4.11 mmol, 1 equiv) and DBU (0.92 mL, 6.2 mmol, 1.5 equiv) in anhydrous DMF (21 mL, 0.2 M) at 0° C. The flask was left to slowly warm in the ice-water bath. After 17 h, the reaction mixture was poured into stirring ethyl acetate-hexanes (5:1, 200 mL) and cold water (200 mL). The organic layer was washed with water (200 mL) and with a saturated aqueous sodium chloride solution (150 mL). The aqueous layers were separately extracted with a single portion of ethyl acetate-hexanes (5:1, 200 mL). The combined organic extracts were dried over anhydrous sodium sulfate, were filtered, and were concentrated under reduced pressure.

STEP 2: The resulting residue was azeotropically dried by concentration from anhydrous toluene (20 mL). The resulting residue was dissolved in DMF (21 mL), cooled to 0° C., then t-butyl bromoacetate (1.52 mL, 10.3 mmol, 2.5 equiv) and sodium hydride (60% dispersion in mineral oil, 329 mg, 8.2 mmol, 2 equiv) were added. The reaction mixture was left in the ice-water bath to slowly warm. After 2 h, the reaction mixture was quenched with methanol (0.5 mL) and then immediately poured into stirring ethyl acetate-hexanes (4:1, 150 mL) and 10% saturated aqueous sodium chloride solution (200 mL). The organic layer was washed with 10% saturated aqueous sodium chloride solution (2×100 mL), and with a saturated aqueous sodium chloride solution (100 mL). The aqueous layers were separately extracted with a single portion of ethyl acetate-hexanes (4:1, 100 mL). The combined organic extracts were dried over anhydrous sodium sulfate, were filtered, and were concentrated under reduced pressure.

STEP 3: The resulting residue was dissolved in THF (25 mL), cooled to 0° C., then TBAF (1.0 M in THF, 10 mL, 10 mmol, 2.5 equiv) was added, then the ice-water bath was removed. After 30 min, the reaction mixture was poured into stirring saturated aqueous sodium bicarbonate solution (100 mL) and DCM (100 mL). The aqueous layer was extracted with DCM (2×100 mL). The last organic layer was reserved for later extractions. The combined first two organic extracts were washed with water (100 mL) and with a saturated aqueous sodium chloride solution (100 mL). The aqueous layers were separately extracted with the reserved DCM layer. The combined organic extracts were dried over anhydrous sodium sulfate, were filtered, and were concentrated under reduced pressure.

STEP 4: The resulting residue was azeotropically dried by concentration from anhydrous toluene (20 mL). The resulting residue was dissolved in DMF (15 mL) and cooled to 0° C., followed by the addition of imidazole (559 mg, 8.2 mmol, 2 equiv) and TBSCl (619 mg, 4.1 mmol, 1 equiv). The ice-water bath was removed, and the reaction was allowed to stir at room temperature. After 1.5 h, additional imidazole (303 mg, 4.5 mmol, 1 equiv) and TBSCl (325 mg, 2.2 mmol, 0.5 equiv) were added. After an additional 30 min, the reaction mixture was poured into stirring ethyl acetate-hexanes (5:1, 150 mL) and cold water (150 mL). The organic layer was washed with water (2×150 mL), and with a saturated aqueous sodium chloride solution (150 mL). The aqueous layers were separately extracted with a single portion of ethyl acetate-hexanes (5:1, 100 mL). The combined organic extracts were dried over anhydrous sodium sulfate, were filtered, and were concentrated under reduced pressure. The resulting residue (5 g) was purified by flash column chromatography on silica gel (50 g, 60 μm, ethyl acetate in hexanes, 0% [2CV], 0→8% [5CV], 8→15% [8CV], 15% [7CV], 15→30% [5CV], 30% [5CV]) to afford alcohol (2.25 g, 90%) as a colorless oil.

### Example 10

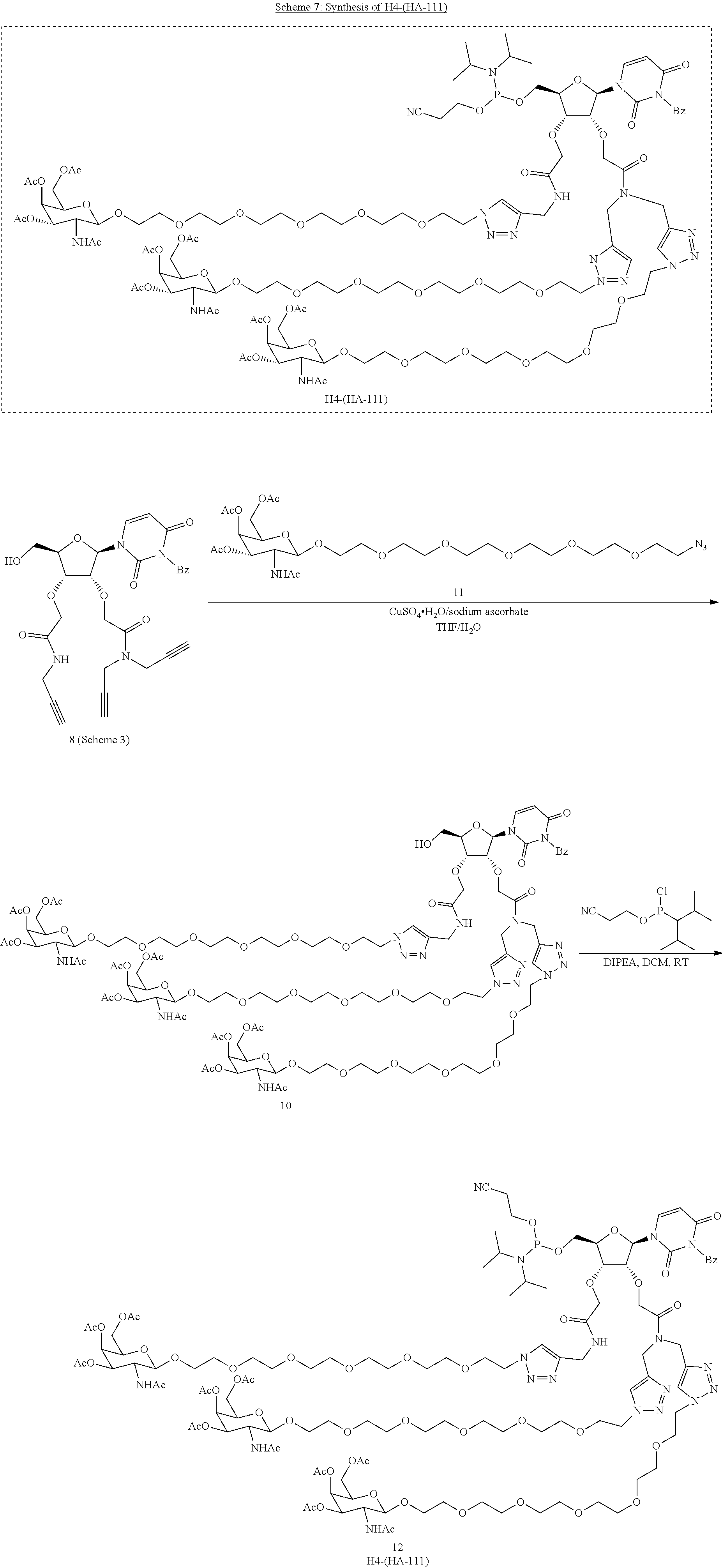

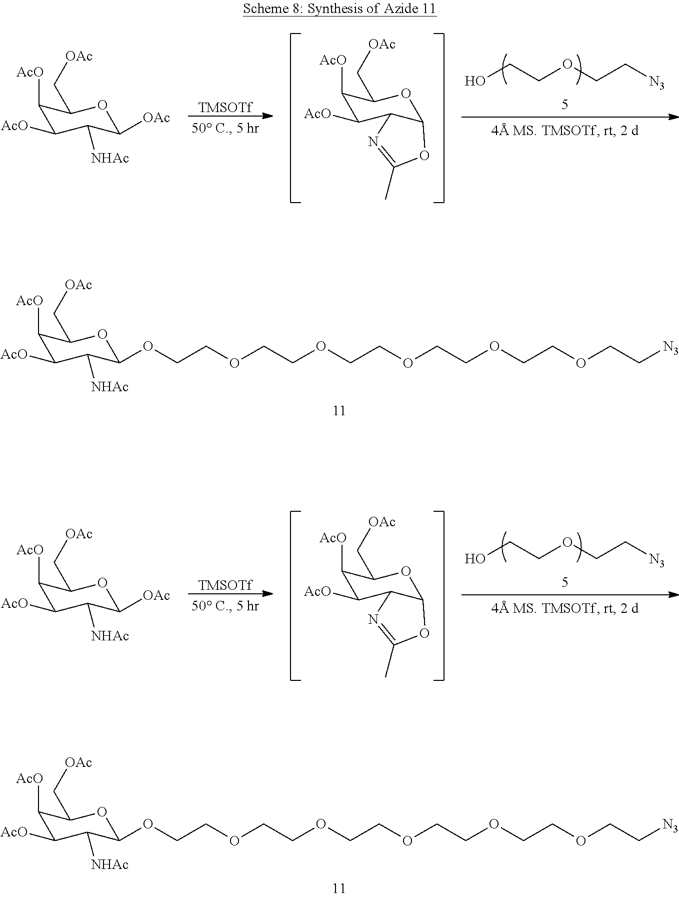

To a suspension of commercially available GalNAc-acetate (11.5 g, 29.579 mmol, 1 eq) in 1,2-dichloroethane (70 mL) at rt was added TMSOTf (6.4 mL, 35.495 mmol, 1.2 eq, d:1.228) over 5 min, then the mixture was warmed up to 50° C. and stirred at 50° C. for 5 hr. The mixture was cooled down to rt, 4 A MS (20 g) was added, then HO-PEG 6 N3 (10.0 g, 32.537 mmol, 1.1 eq) was added. After stirring at rt for 10 min, TMSOTf (1.8 mL, 9.762 mmol, 0.3 eq) was added. The resultant mixture was stirred at rt for 2 days. Work-up: the reaction mixture was filtered and washed with DCM (50 mL). Filtrate was poured into brine (100 mL)+Sat NaHCO₃ (100 mL). Org. layer was separated, Aq. Phase was extracted with DCM (50 mL). The combined Organic layers were washed with brine (100 mL)+Sat NaHCO3 (50 mL), and concentrated. The residue was purified by flash chromatography using 50% EtOAc/Hept to EtOAc to 1%-6% MeOH/EtOAc to obtain azide 11 (10.4 70%) as a pale-yellow syrup.

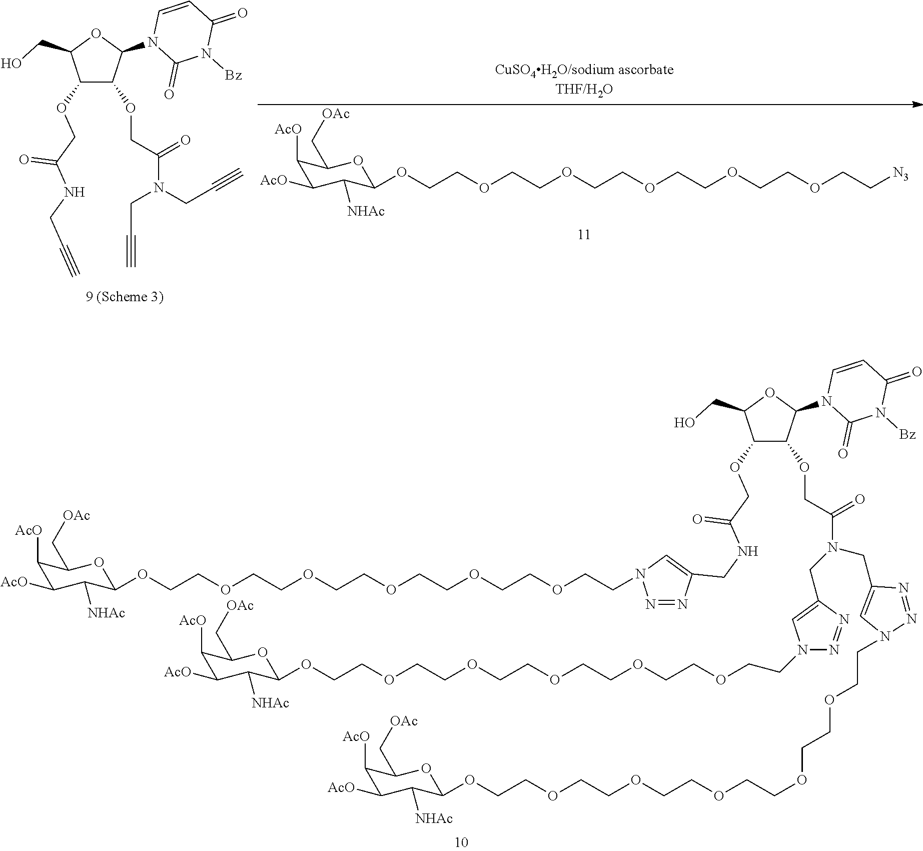

To a solution of compound 9 (1.4 g, 2.428 mmol, 1 eq) and azide 11 (5.1 g, 8.013 mmol, 3.3 eq) in anhydrous THF (28 mL) at 0° C. was added CuSO₄·5H₂O (243 mg, 0.971 mmol, 0.4 eq) in 7 mL water, followed by addition of L-Na-ascorbate (1.457 mmol, 0.6 eq) in 7 mL water, and then the cooling bath was removed. The resultant mixture (pale yellow) was stirred at rt for 5 hr. Work-up: NH4Cl—NH3·H2O (prepared by: 5 g NH₄Cl, 5 mL NH₃·H₂O (29%) in 50 mL water) (20 mL) was added, Aq. phase was extracted with DCM (50 mL×4). The combined organic layers were washed with brine (100 mL)+NH₄Cl—NH₃·H₂O (10 mL) two times, then brine (80 mL)+NH₃·H₂O (1 ml) until Aq. phase showed no blue color, then brine (80 mL). The residue was concentrated and purified by flash chromatography 50% EtOAc/Hept to EtOAc to 1% to 4% MeOH/DCM to give product 10 as a white foam 4.9 g, 81% yield.

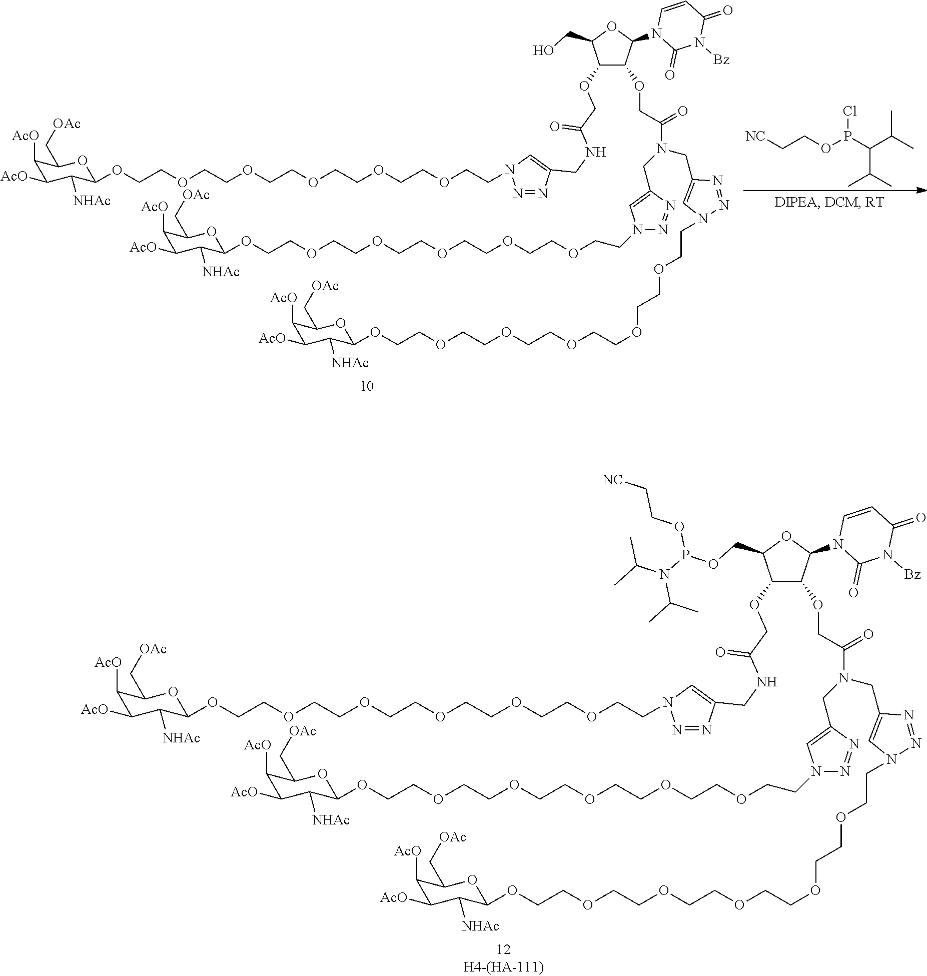

To a stirred solution of alcohol 10 (2.93 g, 1.179 mmol, 1 eq) and diisopropylethylamine (1.23 mL, 7.072 mmol, 6 eq), in DCM (30 mL), was added 2-cyanoethyl N,N-diisopropyl chlorophosphoramidite (0.78 mL 3.536 mmol, 3 eq) dropwise. The reaction mixture was stirred at room temperature for 1 h. Reaction mixture was quenched with Aq. Saturated NaHCO₃ solution (50 mL), extracted with DCM (100) ml, washed with brine (100 mL), dried over N₂SO₄, and evaporated. The crude product was loaded onto (pre-equilibrated two times with 1% Et₃N-DCM) Biotage silica gel column (50 g 20 μm) and purified by flash chromatography using 0-10% MeOH/DCM containing 1% Et₃N as an additive. Pure fractions were combined, concentrated, and dried under high vacuum to obtain Phosphoramidite 12 (2.8 g, 88%) as a beige solid. 92% purity by HPLC, P31-NMR, Mass (m/z 2708 M+Na), H1-NMR correspond with product.

### Example 11

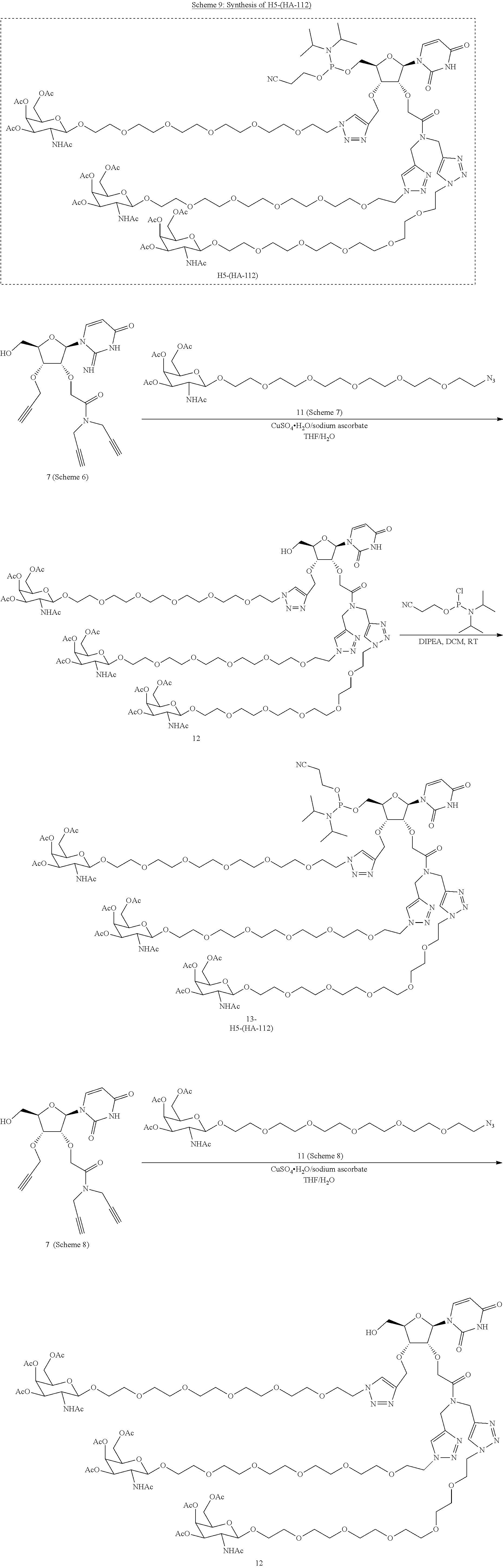

To a stirred solution of alkyne 7 (0.65 g, 1.566 mmol, 1 eq) and azide 11 (3.18 g, 5.012 mmol, 3.2 eq) in THF (30 mL) were added CuSO₄·5H₂O (0.19 g, 0.788 mmol) in water (5 mL) and Na-ascorbate (0.23 g, 1.198 mmol) in water (5 mL), and the mixture was stirred for 3 h at room temperature. LCMS showed product formation and complete disappearance alkyne. The reaction mixture was diluted with aq. Saturated NaHCO₃ (50 mL), extracted with DCM 2×50 mL, and washed with brine solution (50 mL). Combined extracts were dried over Na₂SO₄, and concentrated, resulting crude was purified by column chromatography using 0-30% MeOH/DCM as an eluent, pure fractions were combined, and concentrated resulting solids were co-evaporated with toluene and dried under high vacuum to obtain tris-triazole (2.8 g 76%) as a white solid. LCMS and NMR correspond with product.

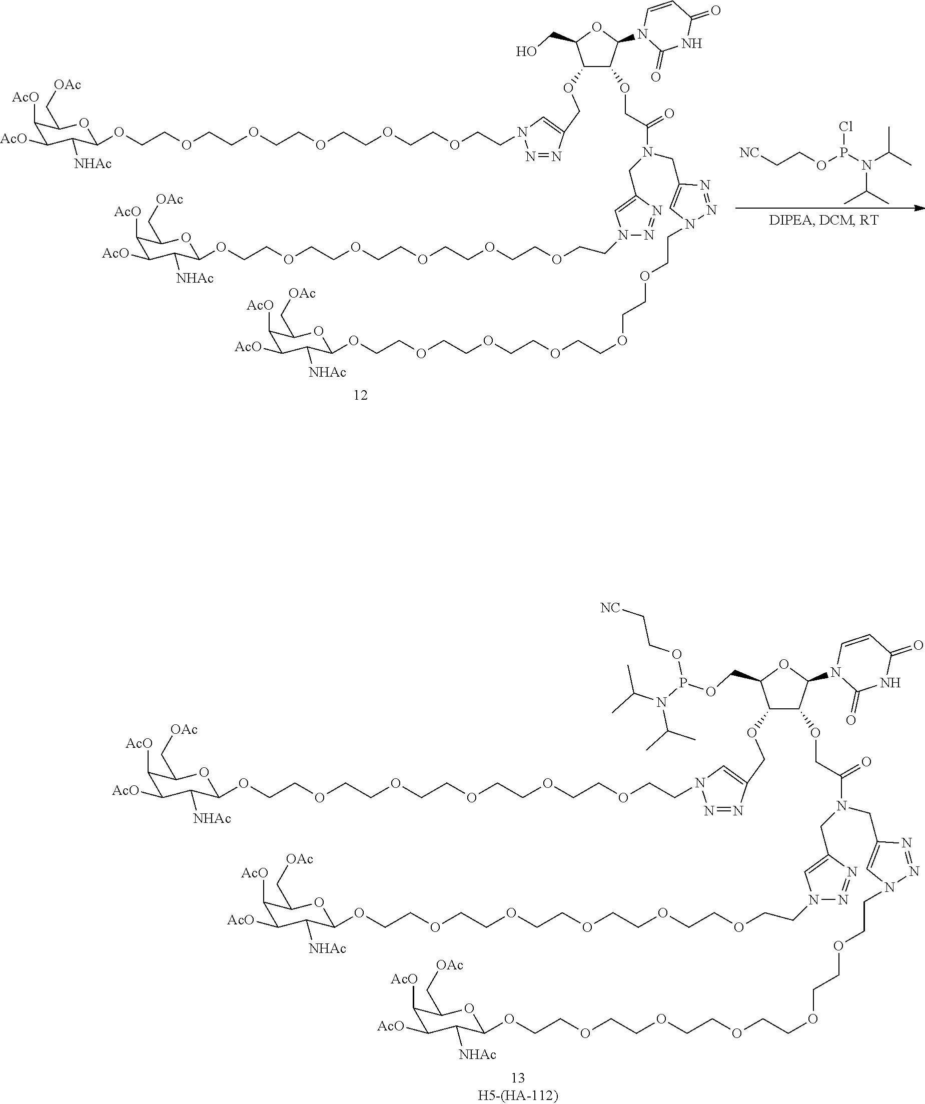

To a stirred solution of alcohol 12 (1.033 g, 0.444 mmol, 1 eq) and diisopropylethylamine (0.46 mL, 2.666 mmol, 6 eq), in DCM (10 mL), was added 2-cyanoethyl N,N-diisopropyl chlorophosphoramidite (0.29 mL 1.33 mmol, 3 eq) dropwise. The reaction mixture was stirred at room temperature for 1 h. Reaction mixture was quenched with Aq. Saturated NaHCO₃ solution (50 mL), extracted with DCM (100 ml), washed with brine (100 mL), dried over Na₂SO₄, and the evaporated crude product was loaded onto (pre-equilibrated with 1% Et3N-DCM) Biotage silica gel column (25 g 20 μm) and purified by flash chromatography using 0-10% MeOH/DCM containing 1% Et₃N as an additive. Pure fractions were combined, concentrated, and dried under high vacuum to obtain Phosphoramidite 13 (1.01 g, 90%) as a beige solid. 91% purity by HPLC, P31-NMR, Mass (m/z 2546 M−1+ Na), and H1-NMR correspond with product.

### Example 12

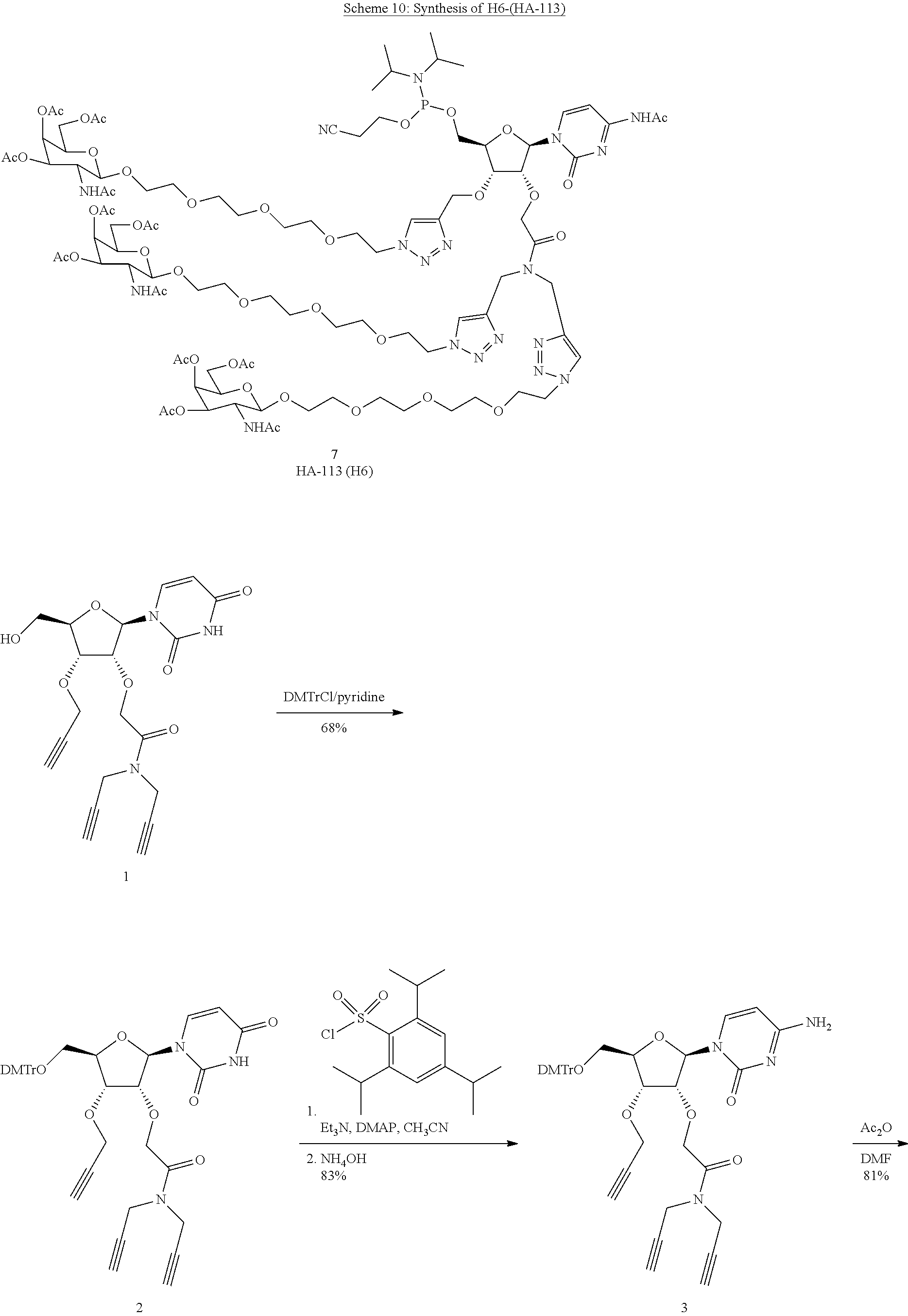

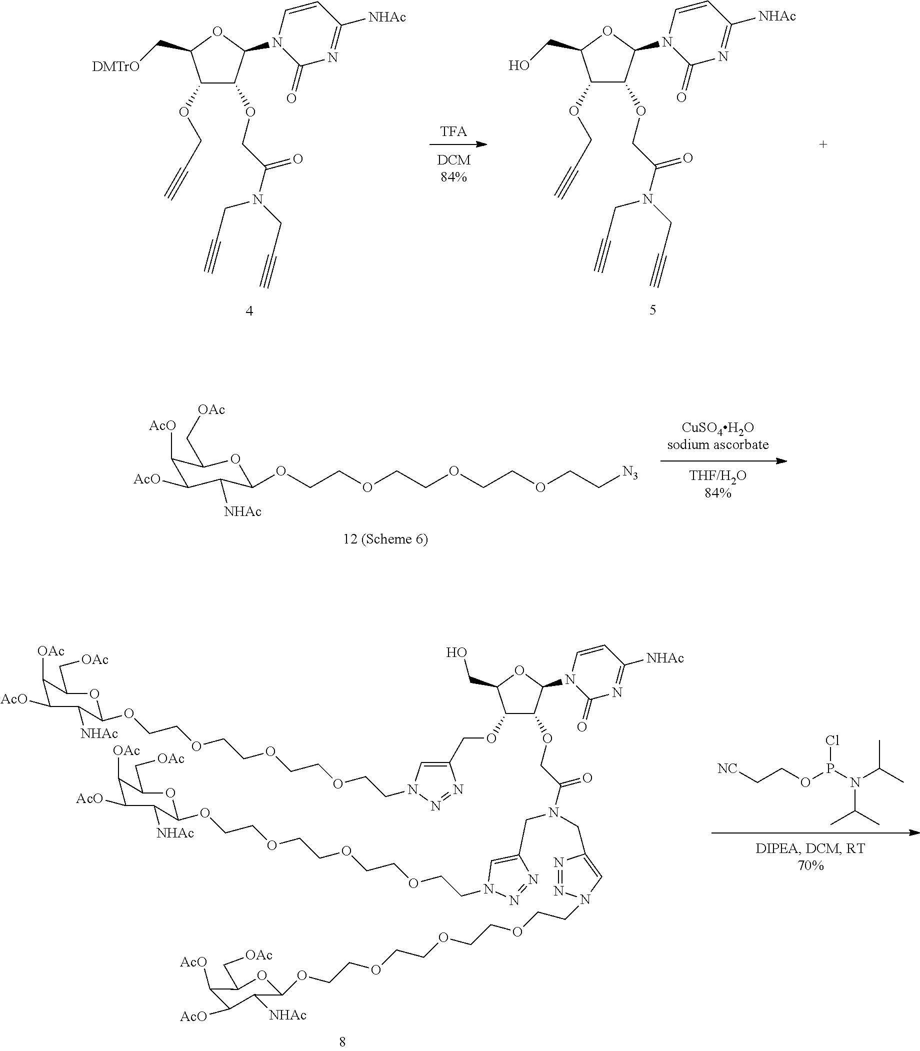

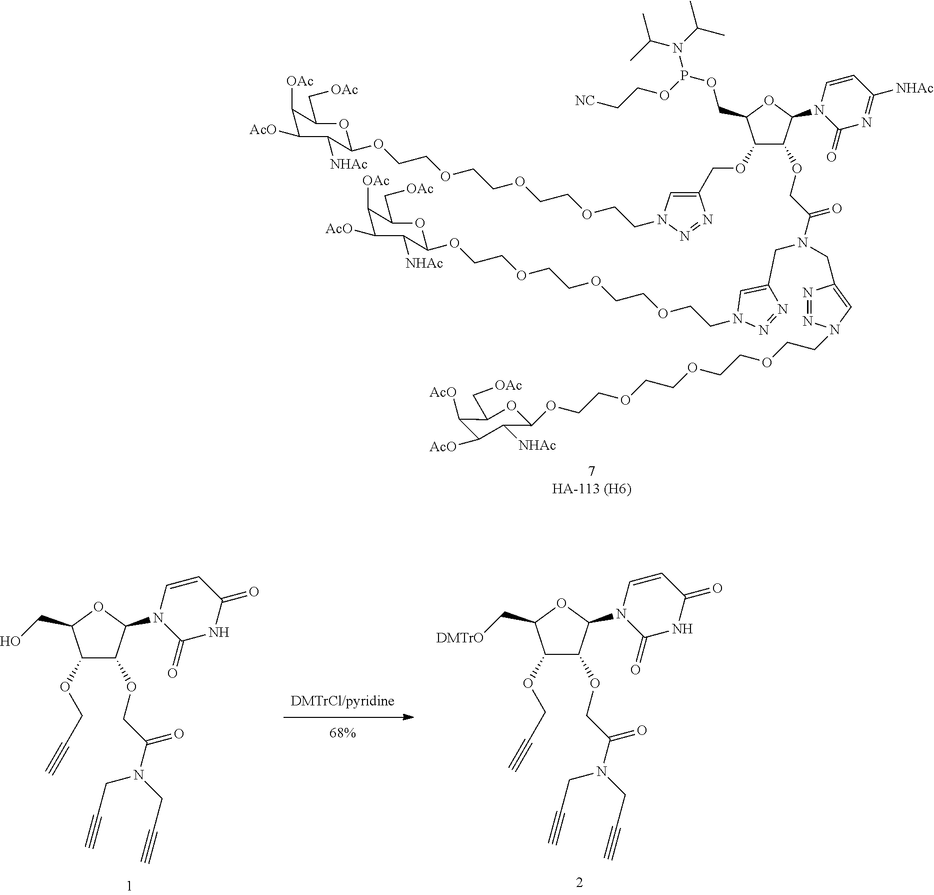

To a stirred solution of the alcohol 1 (0.85 g, 1.974 mmol, 1 eq) in anhydrous pyridine (10 mL) was added DMTrCl (0.87 g, 2.566 mmol, 1.3 eq). The reaction was stirred at rt overnight. MeOH was added to quench the reaction. The crude reaction was concentrated under reduced pressure. The residue was partitioned between EtOAc and NaHCO₃. The organic layer was dried over Na₂SO₄, filtered, and concentrated. The residue was purified on a biotage column (25 g, 20 uM) using 0-60% EtOAc in hexanes (in 10 CV) to afford 0.96 g product 2 (68%) as a slightly yellow solid.

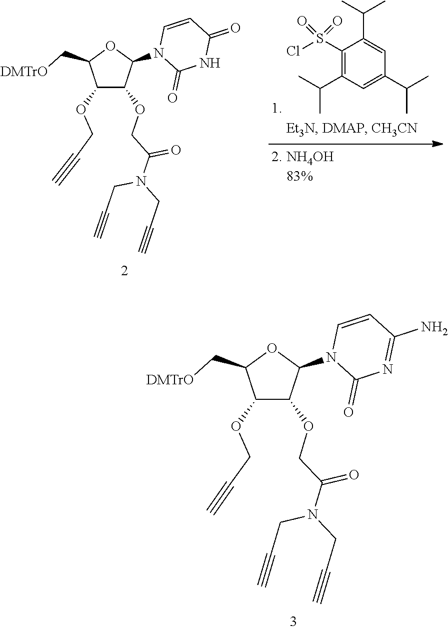

To a stirred solution of the compound 2 (0.8 g, 1.115 mmol, 1 eq) in anhydrous CH₃CN (10 mL) was added DMAP (272 mg, 2.229 mmol, 2 eq), triethylamine (0.311 mL, 2.229 mmol, 2 eq) and TPSCl (675 mg, 2.229 mmol, 2 eq). The reaction was stirred at rt for 2 h. Concentrated ammonium hydroxide (50% v/v in H2O) (5 mL) was added to the reaction. The reaction was stirred overnight. The crude reaction was concentrated under reduced pressure. The residue was partitioned between DCM and sat. NH₄Cl. The aqueous layer was extracted with DCM (1×). The organic layers were collected, dried over Na₂SO₄, filtered, and concentrated. The residue was purified on a biotage column (25 g, 20 uM) using 0-8% MeOH in DCM in 10 CV to give 661 mg product 3 (83%) as an off-white solid.

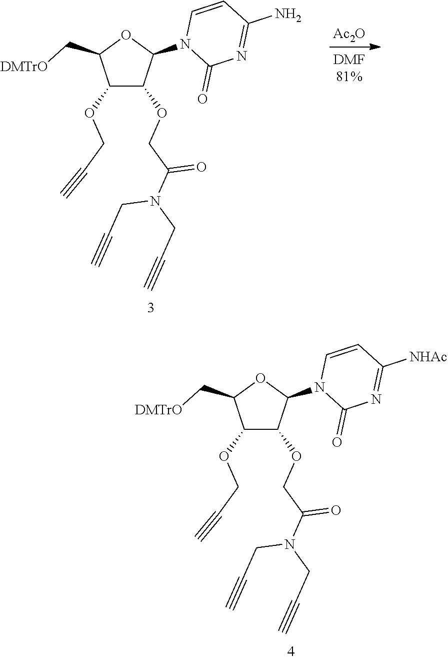

To a solution of amine 3 (660 mg, 0.921 mmol, 1 eq) in anhydrous DMF (5 mL) was added acetic anhydride (0.113 mL, 1.197 mmol, 1.3 eq). The reaction was stirred for 60 h at room temperature. The reaction was diluted with DCM and washed NaHCO₃ (1×). The organic layer was dried with Na₂SO₄, filtered, and concentrated in vacuo to remove solvent. The residue was purified by silica gel chromatography (25 g, 20 μm) using 0-10% MeOH/DCM in 15 CV. Pure fractions were combined, concentrated, and dried under high vacuum to obtain 550 mg product 3 (81%) as a white solid.

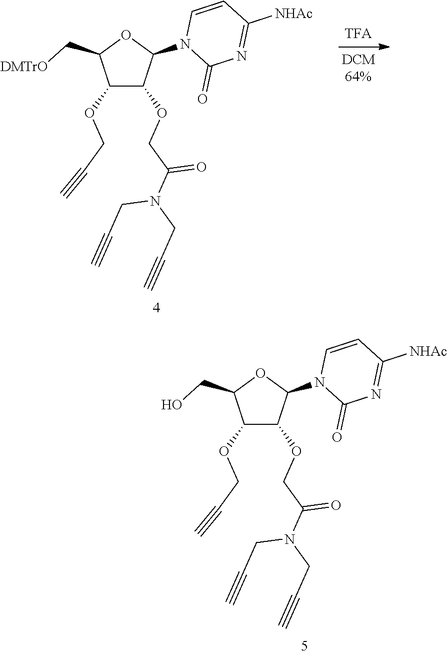

To a solution of DMT ether 4 (0.61 g, 0.804 mmol, 1.00 eq) in DCM (5 mL) at RT was added TFA (0.185 mL, 2.4 mmol, 3.0 eq). The reaction was stirred at RT for 60 min. Additional 0.1 mL TFA was added to the reaction, and the reaction was stirred for 30 min. The reaction was carefully quenched by addition of aq. NaHCO₃. The reaction mixture was partitioned between Saturated NaHCO₃ and DCM. The aqueous phase was extracted with DCM (1×). The organic layers were combined, dried over Na₂SO₄, filtered, and concentrated. The residue was purified by column chromatography on silica gel using 0-10% MeOH in DCM to give 235 mg product 5 (64%) as a white solid.

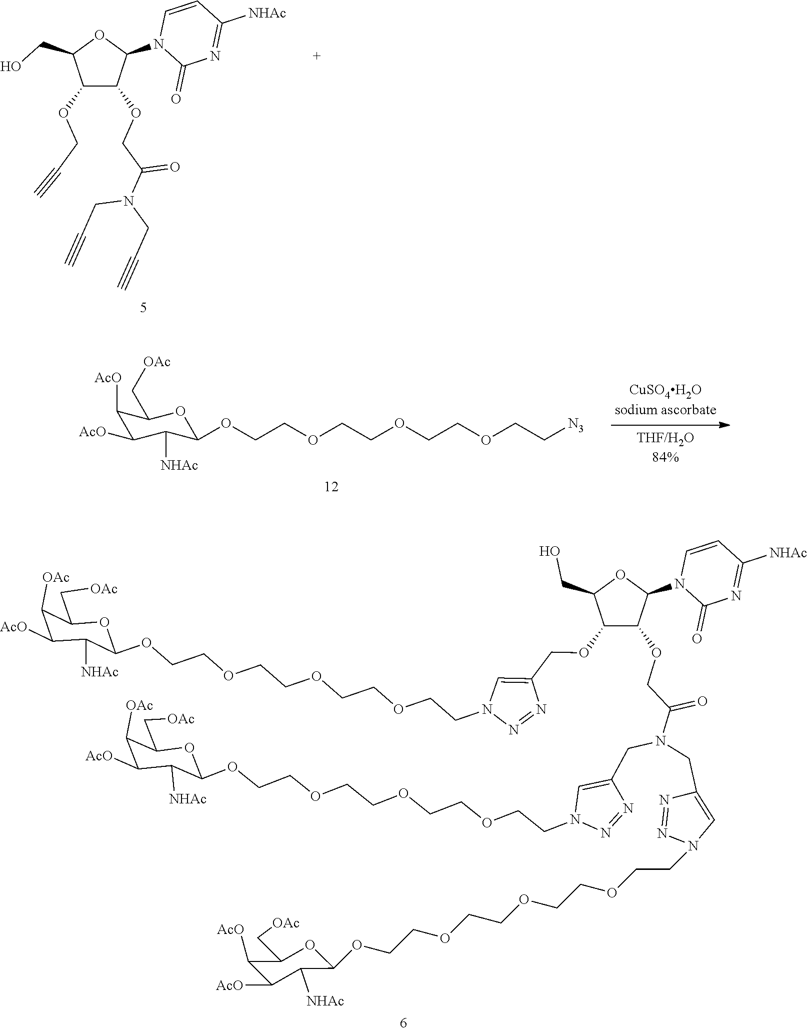 [SMILES: C#CCO[C@H]1[C@@H](OCC(=O)N(CC#C)CC#C)[C@H](n2ccc(NC(C)=O)nc2=O)O[C@@H]1CO ; CC(=O)NC1C(OCCOCCOCCOCCN=[N+]=[N-])OC(COC(C)=O)C(OC(C)=O)C1OC(C)=O ; CC(=O)Nc1ccn([C@@H]2O[C@H](CO)[C@@H](OCc3cn(CCOCCOCCOCCOC4OC(COC(C)=O)C(OC(C)=O)C(OC(C)=O)C4NC(C)=O)nn3)[C@H]2OCC(=O)N(Cc2cn(CCOCCOCCOCCOC3OC(COC(C)=O)C(OC(C)=O)C(OC(C)=O)C3NC(C)=O)nn2)Cc2cn(CCOCCOCCOCCOC3OC(COC(C)=O)C(OC(C)=O)C(OC(C)=O)C3NC(C)=O)nn2)c(=O)n1]

To a solution of compound 5 (0.22 g, 0.482 mmol, 1 eq) and azide 12 (0.846 g, 1.542 mmol, 3.2 eq) in THF (4 mL) at 0° C. was added a solution of CuSO₄·5H₂O (0.036 g, 0.145 mmol, 0.3 eq) in water (1 mL) followed by a solution of sodium ascorbate (0.043 g, 0.217 mmol, 0.45 eq) in water (1 mL). After stirring for 5 min at 0° C., the reaction was warmed to RT. The reaction was stirred for 2 hrs at RT, and LCMS showed the major product with some double-clicked product (m/z: 777). Additional azide 2 (0.132 g, 0.241, 0.5 eq) was added to the reaction. The reaction was stirred for 2 hours at RT. LCMS showed completion of the reaction. The reaction was diluted with DCM and washed with NaHCO₃. The aqueous phase was extracted with DCM (2×). The organic layers were combined, dried over Na₂SO₄, filtered, and concentrated. The residue was purified by 0-20% MeOH in DCM (25 g, 20 uM, in 15 CV) to give 0.85 g triazole 6 (84%) as a white solid.

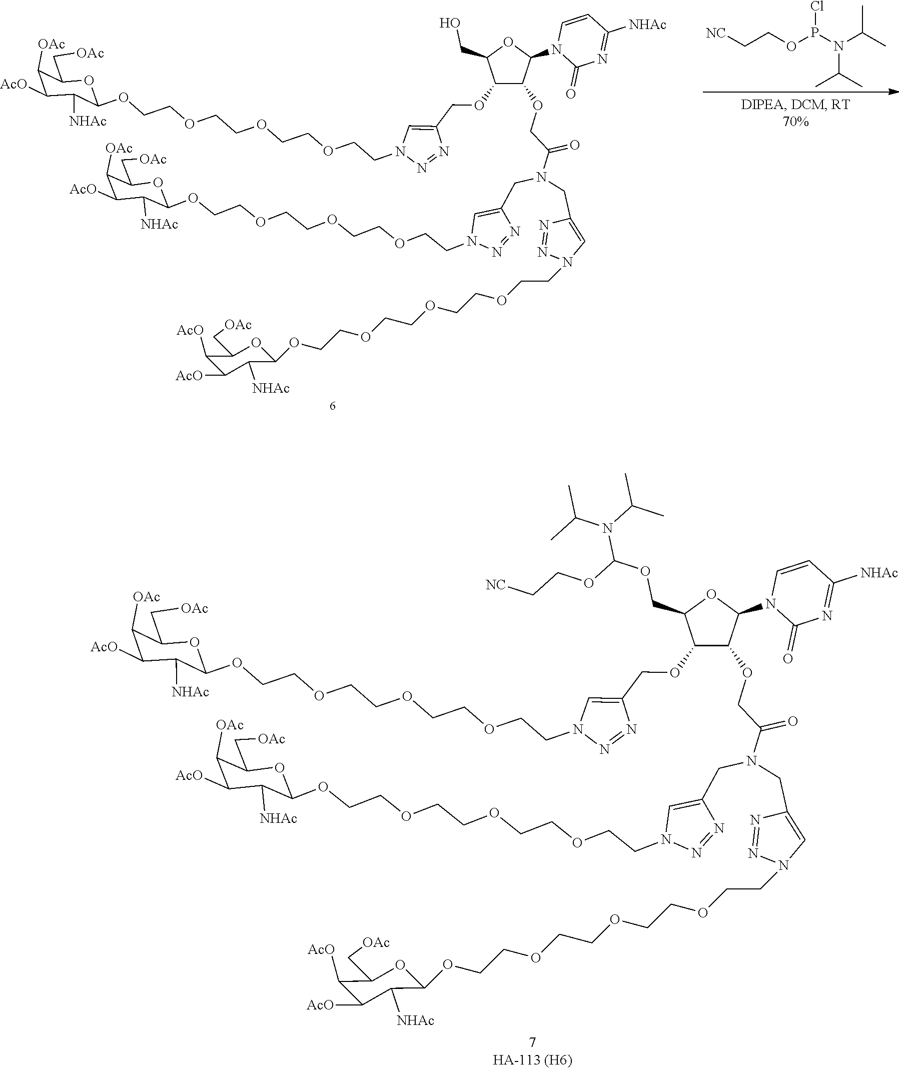

To a stirred solution of triazole 6 (0.78 g, 0.371 mmol, 1 eq) and diisopropylethylamine (0.42 mL, 2.412 mmol, 6.5 eq) in anhydrous DCM (15 mL) was added 2-cyanoethyl N,N-diisopropyl chlorophosphoramidite (0.248 mL, 1.113 mmol, 3 eq) dropwise. The reaction mixture was stirred at room temperature for 40 min. LCMS and HPLC showed completion of the reaction. The reaction mixture was quenched with saturated NaHCO₃ solution and partitioned between DCM and saturated NaHCO₃. The DCM phase was collected. The aqueous phase was extracted with DCM (1×). The combined organic phases were washed with brine, dried over Na₂SO₄, filtered, and concentrated. The residue was loaded on to a pre-equilibrated (1% Et₃N-DCM) biotage silica gel column (25 g, 20 μm) and purified by flash chromatography using 0-15% MeOH/DCM (in 15 CV) containing 1% Et₃N as an additive. Pure fractions were combined, concentrated, and dried under high vacuum to give 0.60 g phosphoramidite 7 (98% purity by HPLC) as an off white solid. MS: m/z=2323.7 [M+Na]⁺. P31-NMR and H1-NMR correspond with product.

### Example 13

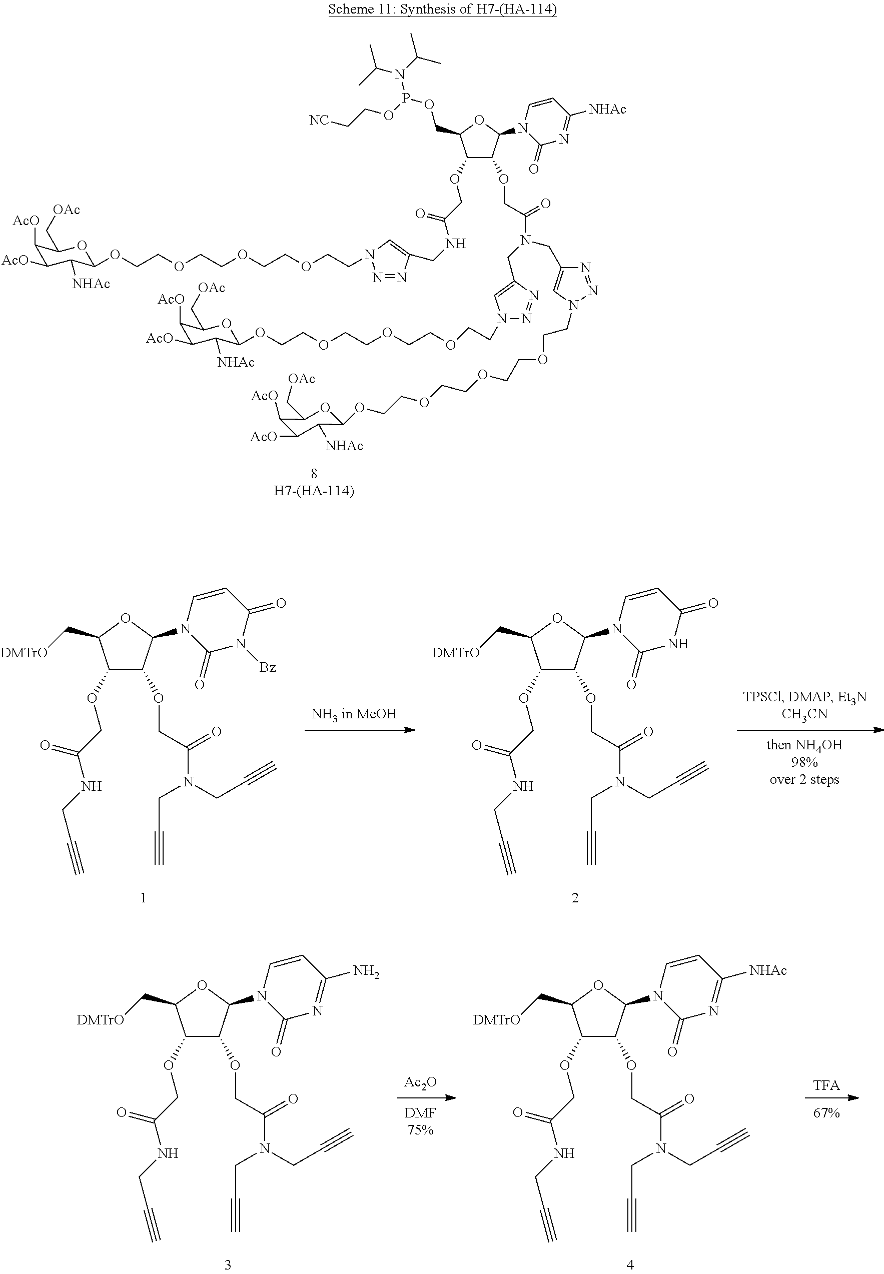

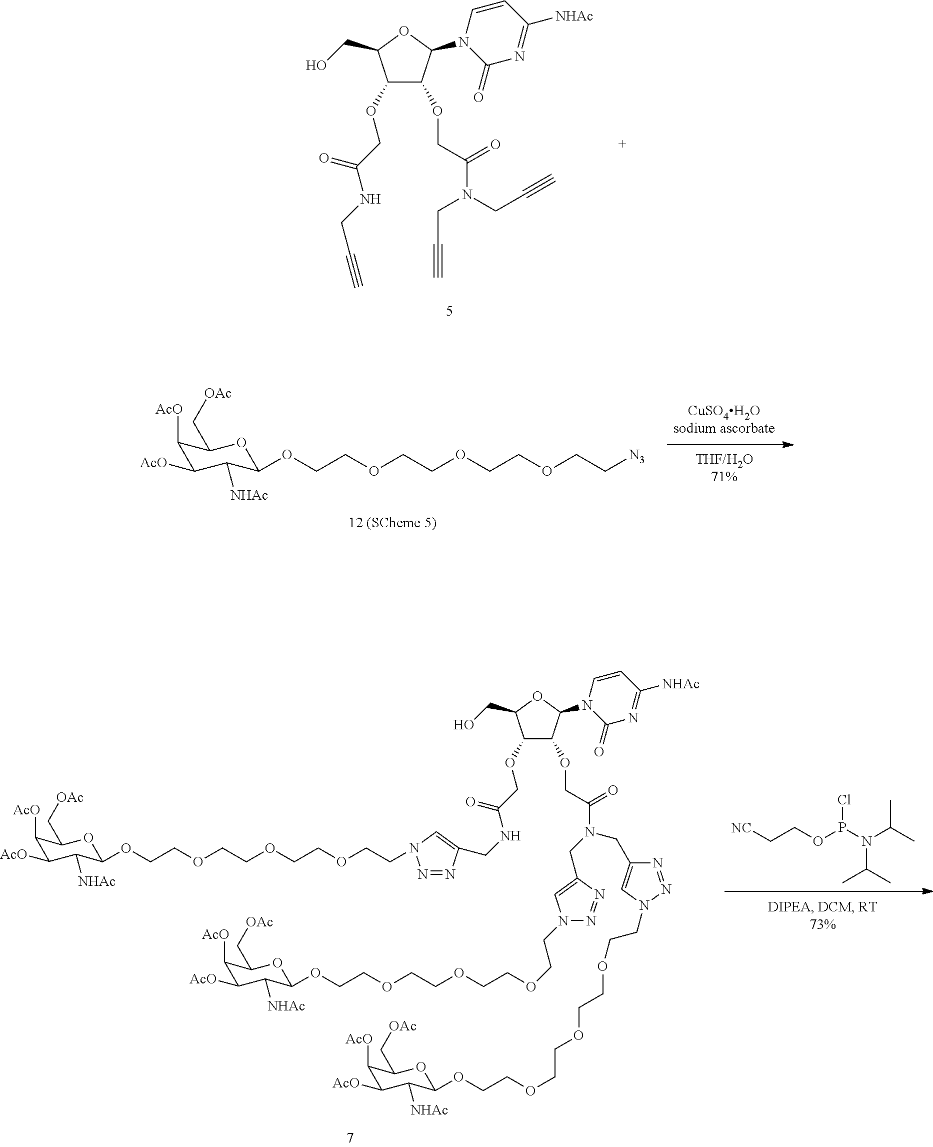 [SMILES: C#CCNC(=O)CO[C@H]1[C@@H](OCC(=O)N(CC#C)CC#C)[C@H](n2ccc(NC(C)=O)nc2=O)O[C@@H]1CO ; CC(=O)NC1C(OCCOCCOCCOCCN=[N+]=[N-])OC(COC(C)=O)C(OC(C)=O)C1OC(C)=O ; CC(=O)Nc1ccn([C@@H]2O[C@H](CO)[C@@H](OCC(=O)NCc3cn(CCOCCOCCOCCOC4OC(COC(C)=O)C(OC(C)=O)C(OC(C)=O)C4NC(C)=O)nn3)[C@H]2OCC(=O)N(Cc2cn(CCOCCOCCOCCOC3OC(COC(C)=O)C(OC(C)=O)C(OC(C)=O)C3NC(C)=O)nn2)Cc2cn(CCOCCOCCOCCOC3OC(COC(C)=O)C(OC(C)=O)C(OC(C)=O)C3NC(C)=O)nn2)c(=O)n1 ; CC(C)N(C(C)C)P(Cl)OCCC#N]

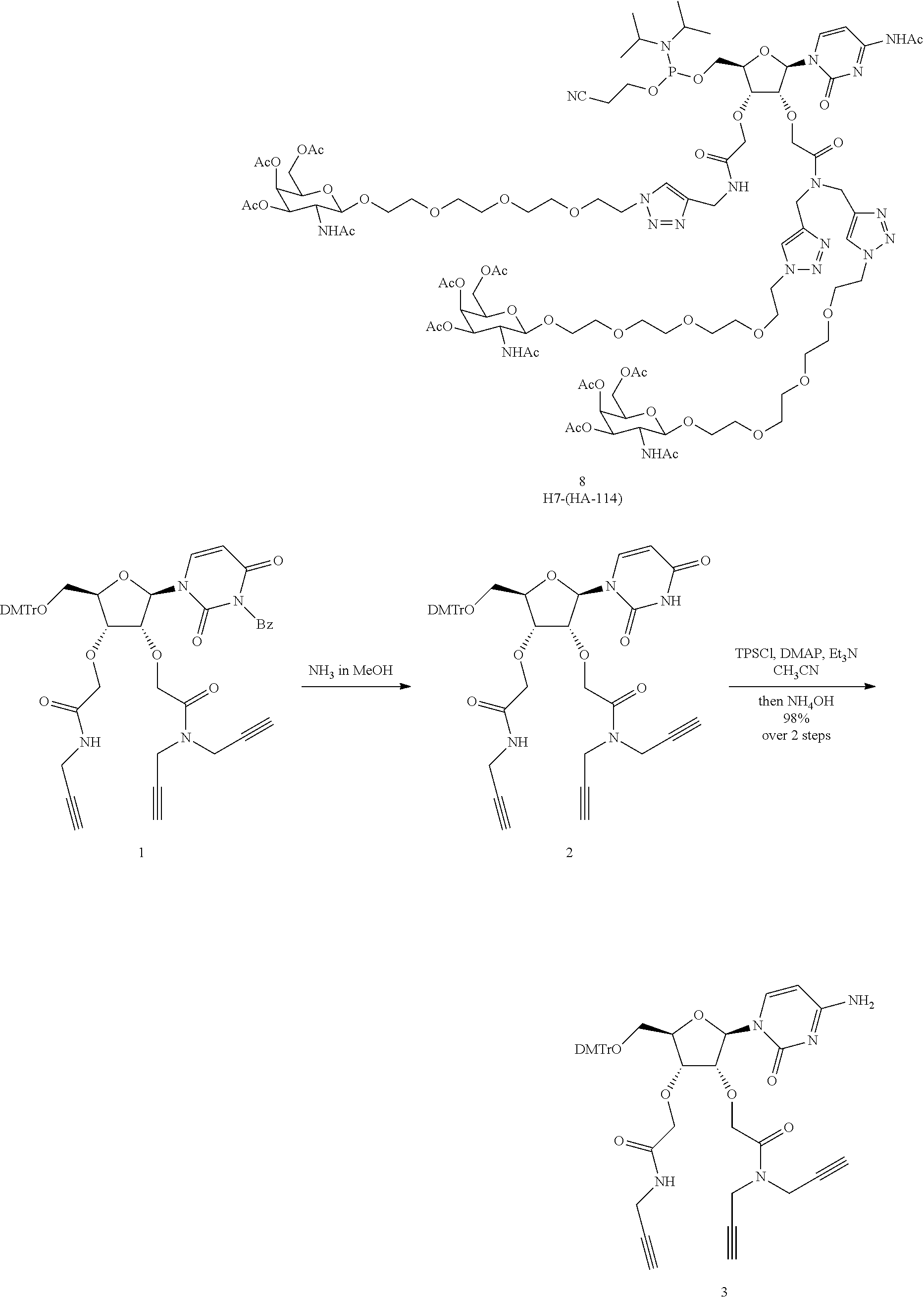

Compound 1 (2.0 g, 2.275 mmol, 1 eq) was dissolved in 7 N NH₃ MeOH (20 mL). The reaction was stirred at RT for 2 h. LCMS showed the reaction was complete. The reaction was concentrated under reduced pressure. The residue was purified by silica gel chromatography (50 g, 20 μm) using 0-10% MeOH/DCM in 15 CV. Pure fractions were combined, concentrated, and dried under high vacuum to obtain 1.82 g product 2 as a white solid. To a stirred solution of the compound 2 (1.82 g, 2.349 mmol, 1 eq) in anhydrous CH₃CN (20 mL) was added DMAP (574 mg, 4.698 mmol, 2 eq), triethylamine (0.655 mL, 4.698 mmol, 2 eq) and 2,4,6-Triisopropylbenzenesulfonyl chloride (1.423 g, 0.468 mmol, 2 eq). The reaction was stirred at rt for 45 min. Concentrated ammonium hydroxide (50% v/v in H₂O) (5 mL) was added to the reaction. The reaction was stirred overnight. The crude reaction was concentrated under reduced pressure. The residue was partitioned between DCM and sat. NH₄Cl. The aqueous layer was extracted with DCM (1×). The organic layers were collected, dried over Na₂SO₄, filtered, and concentrated. The residue was purified by biotage column (50 g, 20 uM) using 0-10% MeOH in DCM in 15 CV to give 1.75 g product 3 (99%) as a white solid.

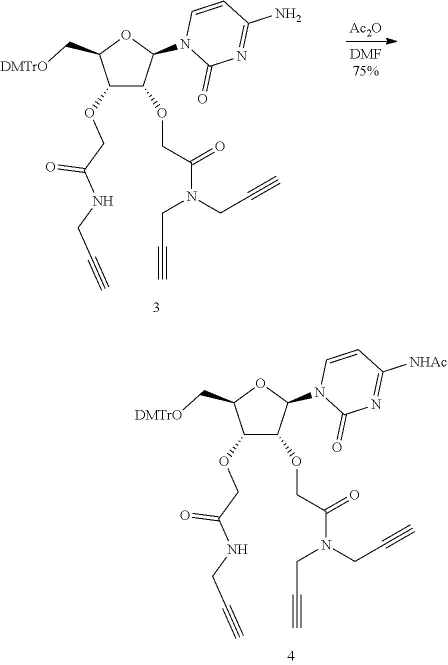

To a solution of compound 3 (1.75 g, 2.261 mmol, 1 eq) in anhydrous DMF (10 mL) was added acetic anhydride (0.278 mL, 2.94 mmol, 1.3 eq). The reaction was stirred for 60 h at room temperature. LCMS shows small amount of SM left. The reaction was diluted with DCM and washed with NaHCO₃ (1×). The organic layer was dried with Na₂SO₄, filtered, and concentrated in vacuo to remove solvent. The residue was purified by silica gel chromatography (50 g, 20 μm) using 0-10% MeOH/EtOAc in 10 CV. Pure fractions were combined, concentrated, and dried under high vacuum to obtain 1.38 g product 4 (75%) as a white solid.

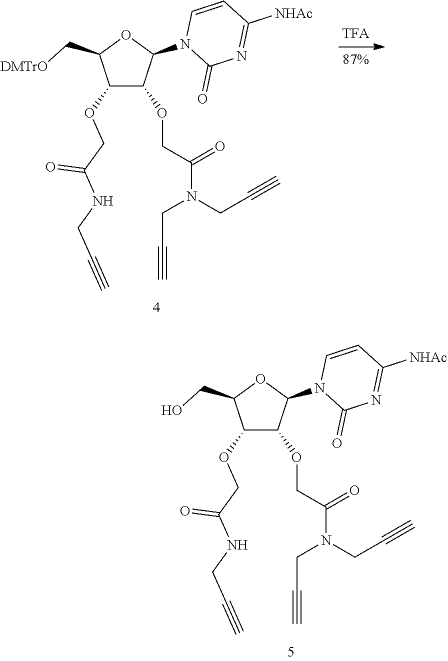

To a solution of DMT ether 4 (1.4 g, 1.716 mmol, 1.00 eq) in DCM (15 mL) at RT was added TFA (0.394 mL, 5.148 mmol, 3.0 eq). The reaction was stirred at RT for 30 min. Additional (0.2 mL) TFA was added to the reaction, and the reaction was stirred for 30 min. The reaction was carefully quenched by addition of aq. NaHCO₃. The reaction mixture was partitioned between Saturated NaHCO₃ and DCM. The aqueous phase was extracted with DCM (1×). The organic layers were combined. White insoluble solids in the organic layers were collected by filtration. LCMS showed that the insoluble solid is product 5. The solid was dried on Vacuum to give 586 mg product 5 (67%) as a white solid.

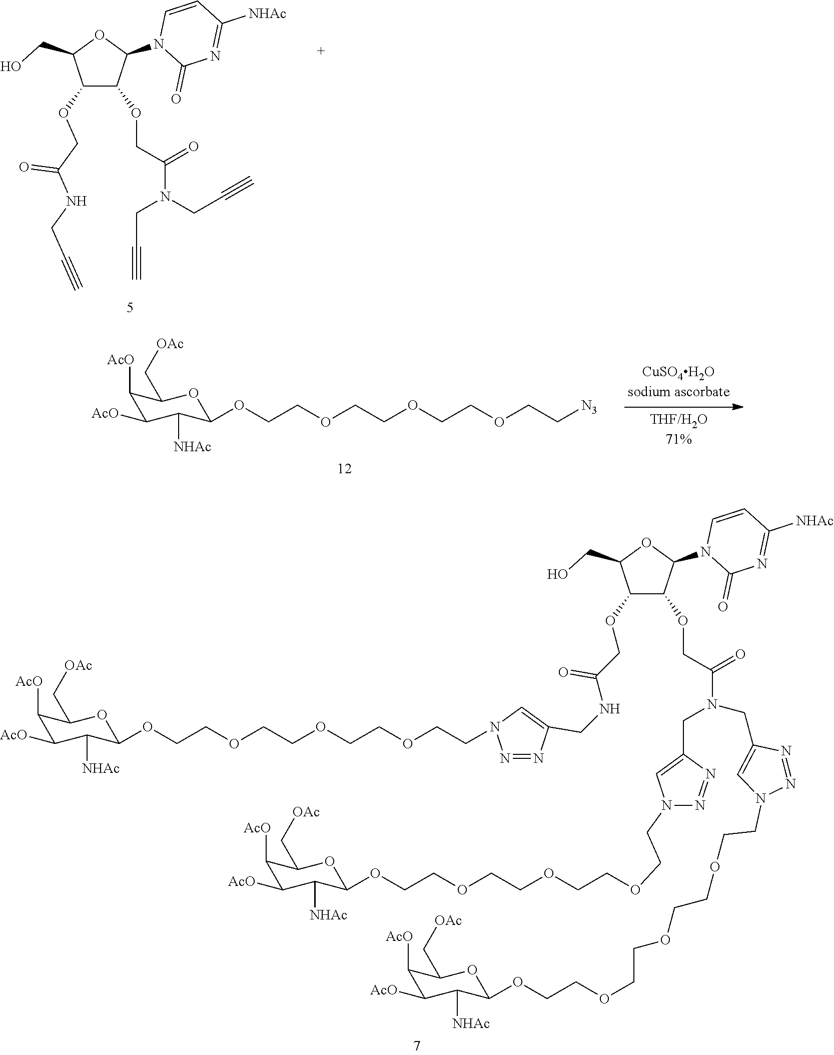 [SMILES: C#CCNC(=O)CO[C@H]1[C@@H](OCC(=O)N(CC#C)CC#C)[C@H](n2ccc(NC(C)=O)nc2=O)O[C@@H]1CO ; CC(=O)NC1C(OCCOCCOCCOCCN=[N+]=[N-])OC(COC(C)=O)C(OC(C)=O)C1OC(C)=O ; CC(=O)Nc1ccn([C@@H]2O[C@H](CO)[C@@H](OCC(=O)NCc3cn(CCOCCOCCOCCOC4OC(COC(C)=O)C(OC(C)=O)C(OC(C)=O)C4NC(C)=O)nn3)[C@H]2OCC(=O)N(Cc2cn(CCOCCOCCOCCOC3OC(COC(C)=O)C(OC(C)=O)C(OC(C)=O)C3NC(C)=O)nn2)Cc2cn(CCOCCOCCOCCOC3OC(COC(C)=O)C(OC(C)=O)C(OC(C)=O)C3NC(C)=O)nn2)c(=O)n1]

To a suspension of compound 12 (0.41 g, 0.798 mmol, 1 eq) and azide 6 (1.445 g, 2.635 mmol, 3.3 eq) in THF (12 mL) at RT was added a solution of CuSO₄·5H₂O (0.060 g, 0.240 mmol, 0.3 eq) in water (3 mL) followed by a solution of sodium ascorbate (0.071 g, 0.359 mmol, 0.45 eq) in water (3 mL). The reaction was stirred for 7 hrs at RT. Then the reaction was diluted with DCM and washed with NaHCO₃. The aqueous phase was extracted with DCM (1×). The organic layers were combined, dried over Na₂SO₄, filtered, and concentrated. The residue was purified by 0-20% MeOH in DCM (50 g, 20 uM, in 15 CV) to give 1.24 g triazole 7 (71%) as a white solid.

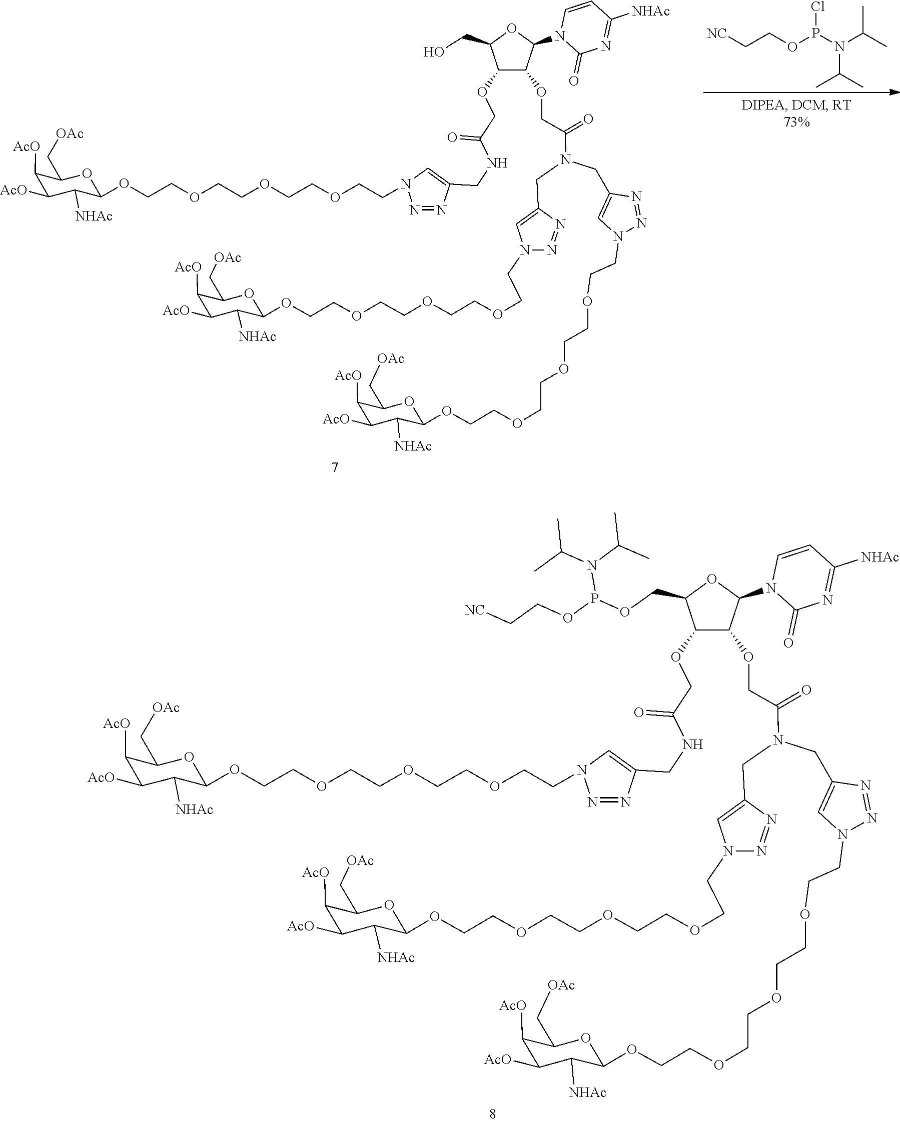

To a stirred solution of triazole 7 (1.63 g, 0.755 mmol, 1 eq) and diisopropylethylamine (0.855 mL, 4.9 mmol, 6.5 eq) in anhydrous DCM (20 mL) was added 2-cyanoethyl N,N-diisopropyl chlorophosphoramidite (0.505 mL, 2.265 mmol, 3 eq) dropwise. The reaction mixture was stirred at room temperature for 40 min. LCMS and HPLC showed completion of the reaction. The reaction mixture was quenched with saturated NaHCO₃ solution and partitioned between DCM and saturated NaHCO₃. The DCM phase was collected. The aqueous phase was extracted with DCM (1×). The combined organic phases were washed with brine, dried over Na₂SO₄, filtered, and concentrated. The residue was loaded on to a pre-equilibrated (1% Et₃N-DCM) biotage silica gel column (50 g, 20 μm) and purified by flash chromatography using 0-15% MeOH/DCM (in 15 CV) containing 1% Et₃N as an additive. Pure fractions were combined, concentrated, and dried under high vacuum to give 1.3 g phosphoramidite 8 (98% purity by HPLC) as an off white solid. MS: m/z=2381.5 [M+Na]⁺. P31-NMR and H1-NMR correspond with product.

### Example 14

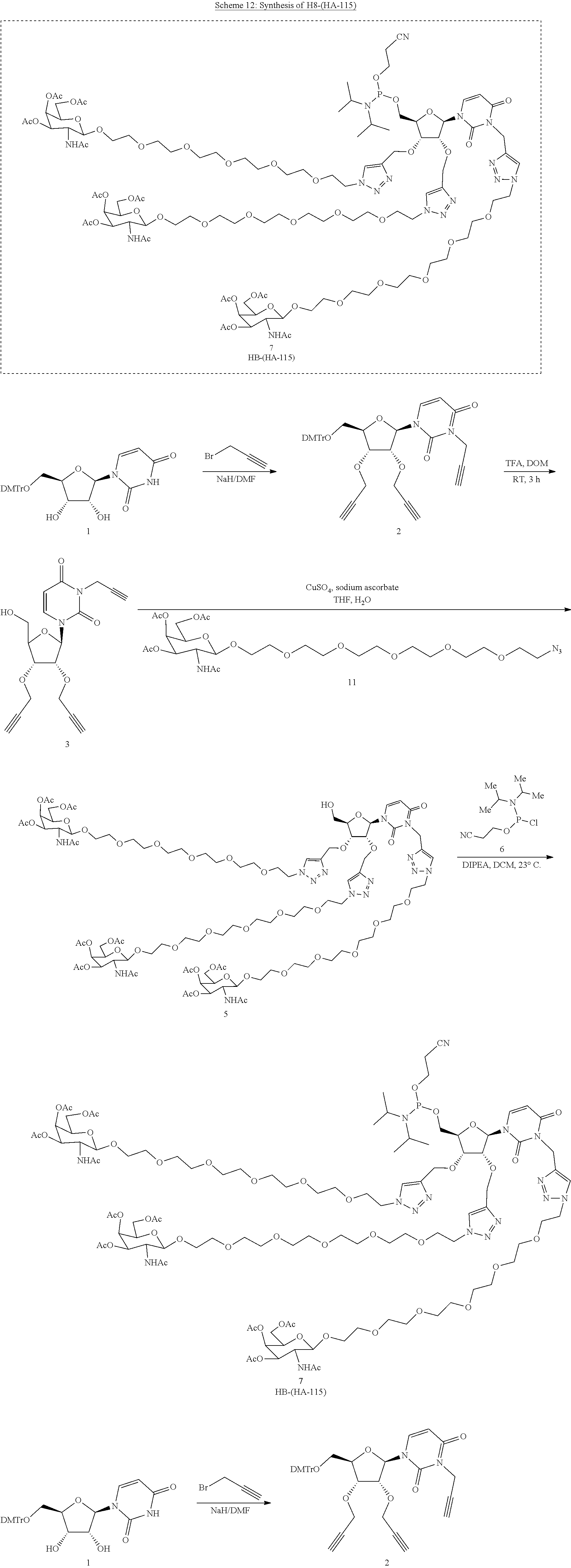

To a stirred and cooled (0° C.) solution of compound 1 (10.08 g, 18.462 mmol, 1 eq) in anhydrous DMF (100 ml) was added NaH 60 w % (2.58 mg 64.615 mmol, 3 eq). The reaction mixture was stirred at 0° C., for 30 min. Propargylbromide (8.236 mL, 55.38 mmol, 3 eq) was added dropwise, and stirring was continued for 2 h at 0° C. The reaction mixture was quenched with water (200 mL), extracted with Ethyl acetate (200 mL), washed with water (2×100 mL) and Brine (100 mL), dried (Na₂SO₄), and concentrated, and the residue was purified by silica gel column chromatography using 0-70% Ethyl acetate/hexane as an eluent. Pure fractions were combined and concentrated to obtain the tri-propargyl product as a white solid (11 g, 90%). Product was confirmed by NMR and LCMS (m/z 684 M+Na).

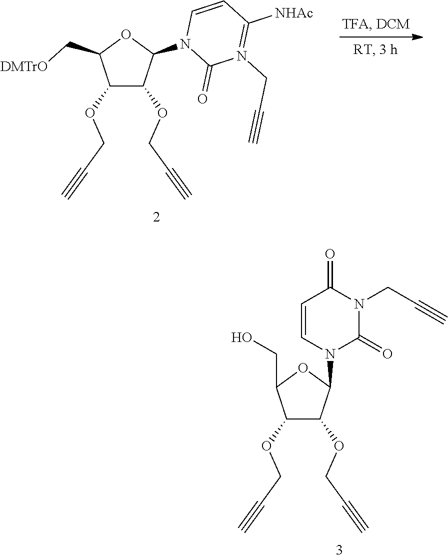

To a stirred and cooled 0° C. solution of DMT ether (10 g, 14.265 mmol, 1.00 eq.) in DCM (50 mL) was added TFA (2.4 mL, 31.384 mmol, 5 eq). The reaction mixture was stirred at RT for 3 h. LCMS showed complete deprotection. The reaction mixture was quenched with NaHCO₃ (50 mL), extracted with DCM (2×200 mL), dried over Na₂SO₄, filtered, and concentrated. The residue was purified by column chromatography on silica gel using 0-100 EtOAc/Hexane as an eluent, and pure fractions were combined and concentrated to obtain alcohol (4.5 g 83%) as a white solid. Product was confirmed by NMR and LCMS. M/z 359 (M+1).

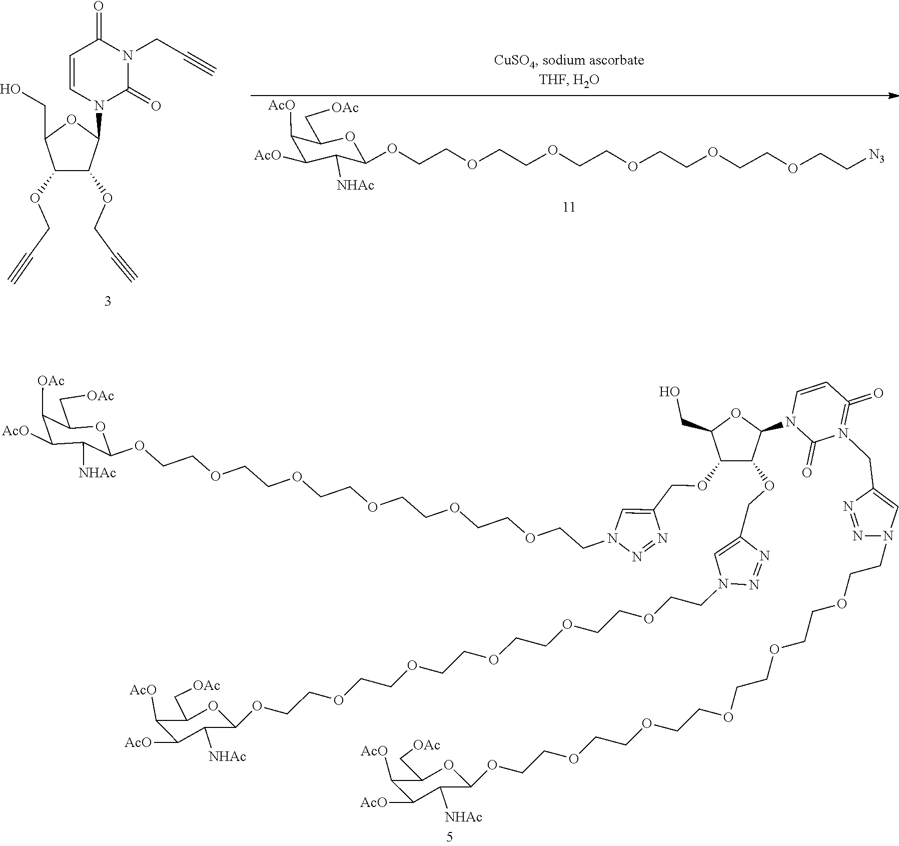

To a stirred solution of Tris-alkyne 3 (1.07 g, 2.98 mmol, 1 equiv) and azide 11 (6.08 g, 9.56 mmol, 3.2 eq) in THF (20 mL) were added CuSO₄·5H₂O (0.37 g, 1.49 mmol, 0.5 eq), in water (2 mL) followed by sodium ascorbate (0.44 g, 2.24 mmol, 0.75 eq) in water (2 mL), and the mixture was stirred for 3 h at room temperature. LCMS showed complete disappearance of alkyne. The reaction mixture was diluted with aq. Saturated NaHCO₃ (50 mL) and extracted with DCM (2×50 mL) and 10% DCM:MeOH (50 ml). The combined organic layers were washed with brine solution 100 mL, dried over Na₂SO₄, and concentrated. The resulting crude product was purified by column chromatography using 0-40% MeOH/DCM as an eluent. Pure fractions were combined and concentrated, and the resulting solids were co-evaporated with toluene and dried under high vacuum to obtain the tris-triazole product (5 g; 72%) as a white solid. LCMS and NMR correspond with product.

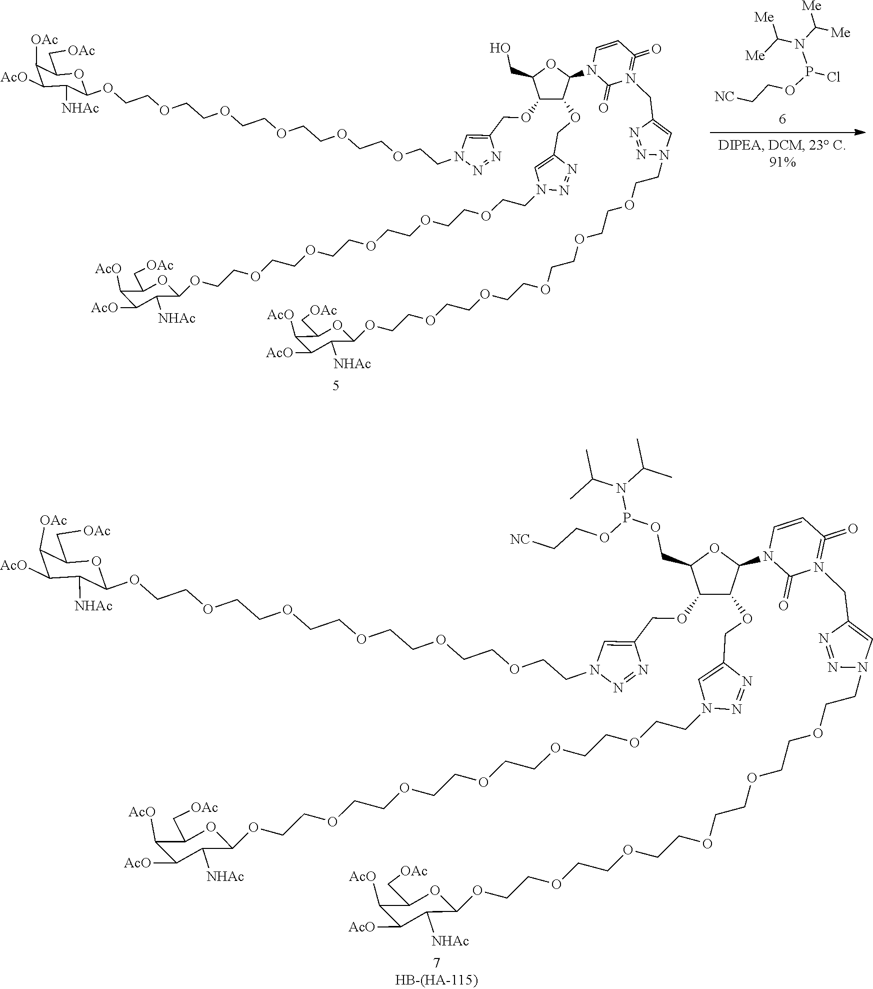

To a stirred solution of alcohol 5 (0.6 g, 0.724 mmol, 1 eq) and diisopropylethylamine (0.199 g, 4.346 mmol, 6 eq), in DCM (20 mL), was added 2-cyanoethyl N, N-diisopropyl chlorophosphoramidite (0.18 g, 0.77 mmol, 3 eq) dropwise. The reaction mixture was stirred at room temperature for 30 min. LCMS showed completion of the reaction. The reaction mixture was quenched with Aq. Saturated NaHCO₃ solution (20 mL), extracted with DCM (2×30 mL), washed with brine (20 mL), and dried over N₂SO₄, and the evaporated crude product was loaded on to pre-equilibrated (1% Et3N-MeOH:1% DCM (1:10)) Biotage silica gel column (50 g 20 μm) and purified by flash chromatography using 0-10% MeOH/DCM containing 1% Et3N as an additive. Pure fractions were collected and concentrated to obtain product 7 0.5 g (77%) as a white solid with >95% HPLC purity. Mass and NMR correspond with product.

### Example 15

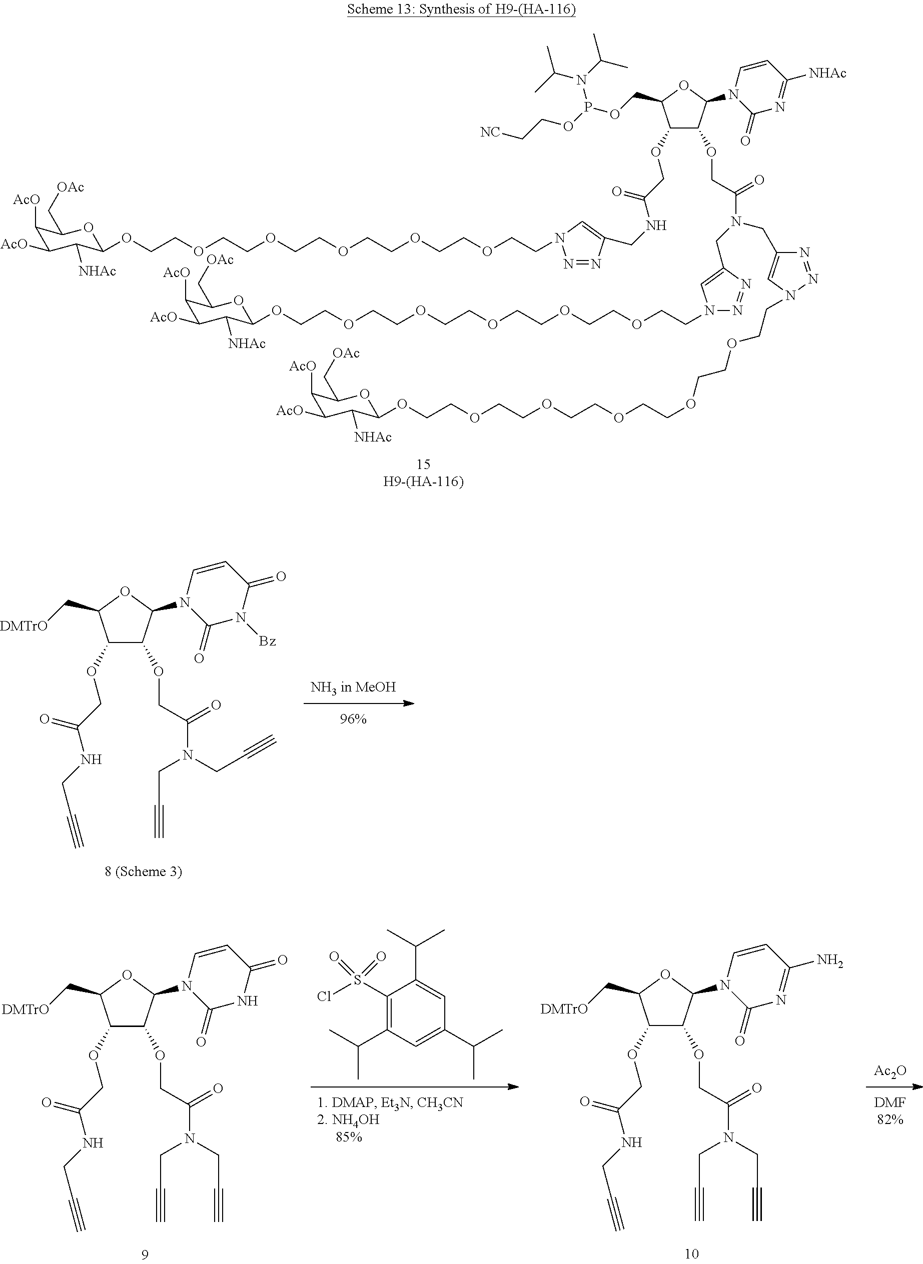

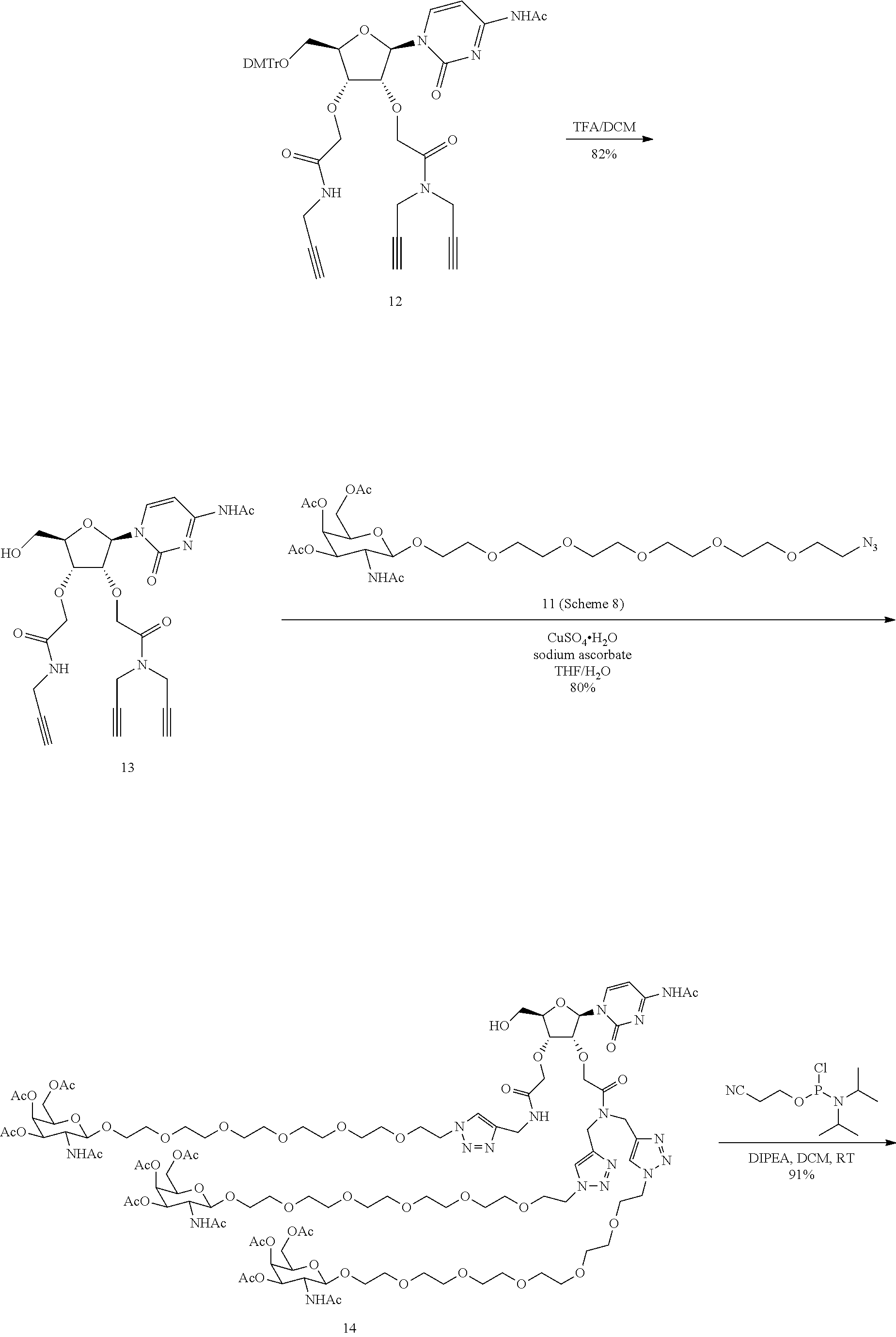

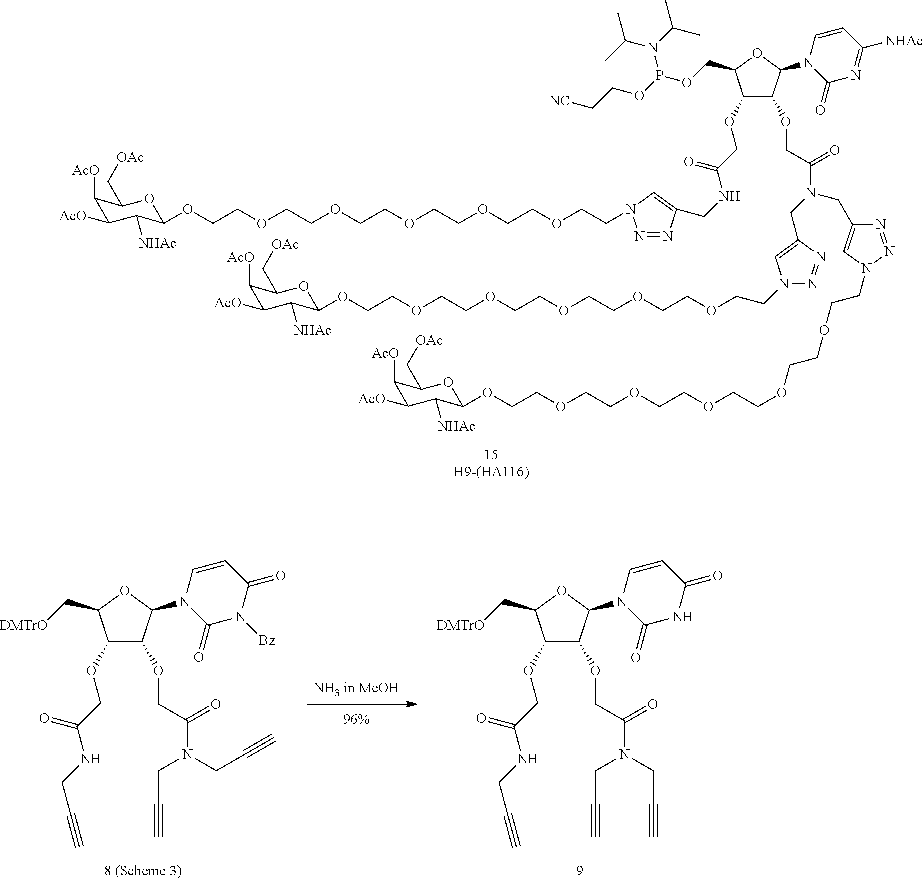

Benzoyl derivative 8 (7.3 g, 8.314 mmol, 1 eq) was dissolved in 7 N NH₃-MeOH (70 mL). The reaction was stirred at RT for 2 h. LCMS showed completion of reaction. The reaction mixture was concentrated under reduced pressure. The residue was purified by silica gel chromatography (100 g, 60 μm) using 0-10% MeOH/DCM in 15 CV. Pure fractions were combined, concentrated, and dried under high vacuum to obtain 6.2 g product (96%) as a white solid. LCMS m/z=775 M+Na and NMR correspond with product.

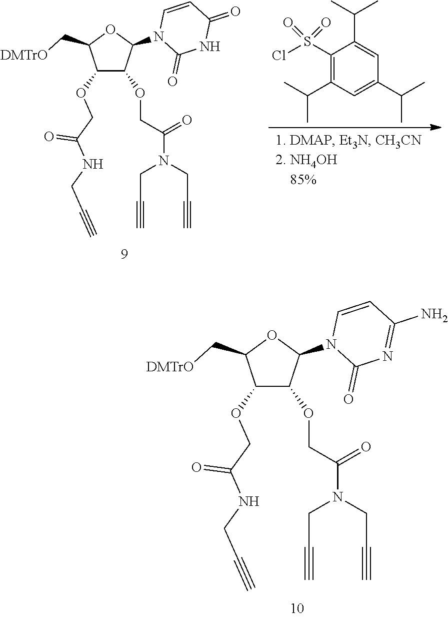

To a stirred solution of the uridine derivative 9 (6.15 g, 7.946 mmol, 1 eq) in anhydrous CH₃CN (60 mL) was added DMAP (1.94 g, 15.891 mmol, 2 eq), triethylamine (2.215 mL, 15.891 mmol, 2 eq), and 2,4,6-Triisopropylbenzenesulfonyl chloride (4.813 g, 15.891 mmol, 2 eq). The reaction was stirred at rt for 1 h. LCMS showed completion of starting material. Concentrated ammonium hydroxide (50% v/v in H2O) (15 mL) was added to the reaction and the mixture was stirred overnight. The crude reaction was concentrated under reduced pressure. The residue was partitioned between DCM and sat. NH₄Cl. The aqueous layer was extracted with DCM (1×200 mL). The organic layers were collected, dried over Na₂SO₄, filtered, and concentrated. The residue was purified by Biotage column (50 g, 20 uM) using 0-10% MeOH in DCM in 15 CV to give 5.2 g product (85%) as a white solid. NMR and LCMS m/z 774 (M+1) correspond with product.

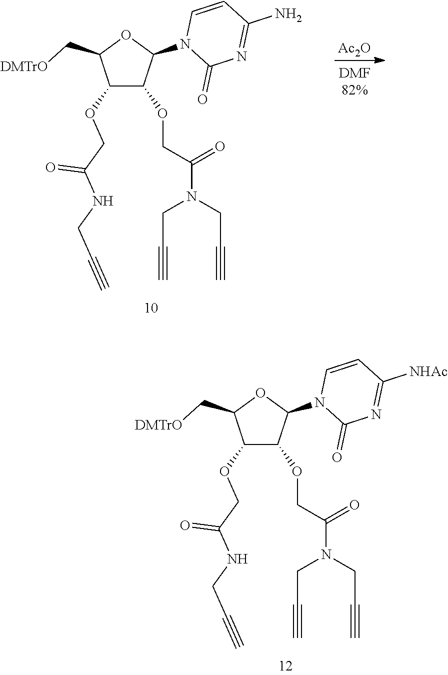

To a solution of amine 10 (5.12 g, 6.624 mmol, 1 eq) in anhydrous DMF (40 mL) was added acetic anhydride (0.75 mL, 7.948 mmol, 1.2 eq). The reaction was stirred for 60 h at room temperature. The reaction was diluted with DCM (300 mL) and washed with NaHCO₃ (100 mL). The organic layer was dried with Na₂SO₄, filtered, and concentrated. The residue was purified by silica gel chromatography (50 g, 20 μm) using 0-10% MeOH/EtOAc in 10 CV. Pure fractions were combined, concentrated, and dried under high vacuum to obtain 4.4 g of acetate (82%) as a white solid. NMR and LCMS m/z 816 (M+1) correspond with product.

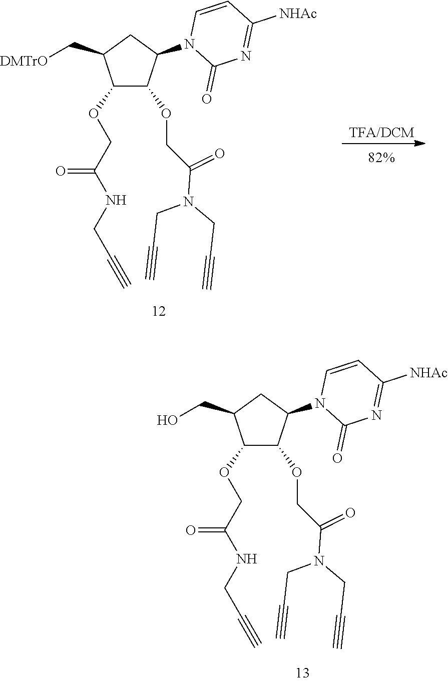

To a solution of DMT ether 12 (4.35 g, 5.337 mmol, 1.00 eq) in DCM (40 mL) at RT was added TFA (1.63 mL, 21.35 mmol, 4.0 eq), and the mixture was stirred at RT for 4 h. The reaction mixture was diluted with DCM (100 mL) and carefully quenched by addition of aq. NaHCO₃ (100 mL). The product was crashed out as a white solid. The solids were collected by filtration, washed with DCM, and dried under high vacuum to give 2.25 g product (82%) as a white solid. NMR and LCMS m/z=514 (M+Na) correspond with product.

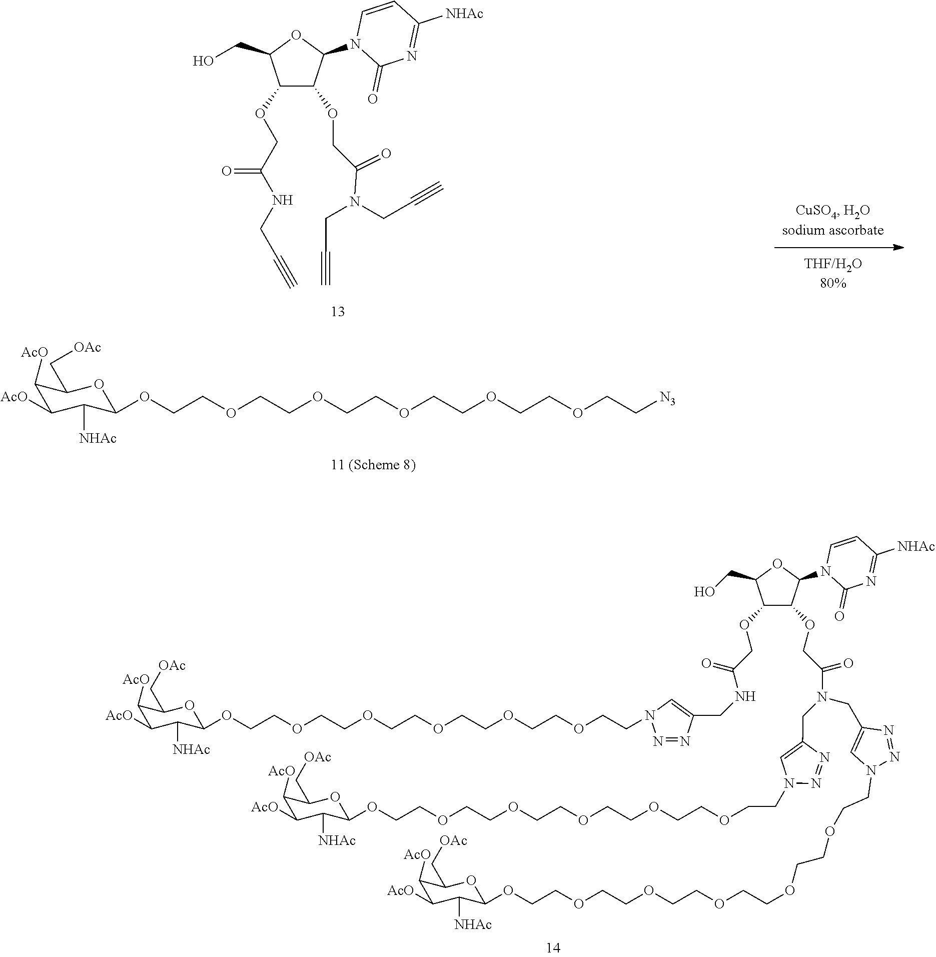 [SMILES: C#CCNC(=O)CO[C@H]1[C@@H](OCC(=O)N(CC#C)CC#C)[C@H](n2ccc(NC(C)=O)nc2=O)O[C@@H]1CO ; CC(=O)NC1C(OCCOCCOCCOCCOCCOCCN=[N+]=[N-])OC(COC(C)=O)C(OC(C)=O)C1OC(C)=O ; CC(=O)Nc1ccn([C@@H]2O[C@H](CO)[C@@H](OCC(=O)NCc3cn(CCOCCOCCOCCOCCOCCOC4OC(COC(C)=O)C(OC(C)=O)C(OC(C)=O)C4NC(C)=O)nn3)[C@H]2OCC(=O)N(Cc2cn(CCOCCOCCOCCOCCOCCOC3OC(COC(C)=O)C(OC(C)=O)C(OC(C)=O)C3NC(C)=O)nn2)Cc2cn(CCOCCOCCOCCOCCOCCOC3OC(COC(C)=O)C(OC(C)=O)C(OC(C)=O)C3NC(C)=O)nn2)c(=O)n1]

To a suspension of compound tri-alkyne 13 (2.2 g, 4.288 mmol, 1 eq) and azide 11 (9 g, 14.152 mmol, 3.3 eq) in THF (50 mL) at RT was added a solution of CuSO₄·5H₂O (535 mg, 2.144 mmol, 0.5 eq) in water (10 mL) followed by a solution of sodium ascorbate (198 mg, 3.216 mmol, 0.75 eq) in water (10 mL). The reaction was stirred for 2 h at RT. Then the reaction was diluted with DCM and washed with NaHCO₃ (100 mL). The aqueous phase was extracted with DCM (2×500 mL). The organic layers were combined, dried over Na₂SO₄, filtered, and concentrated. The residue was purified by 0-20% MeOH in DCM (100 g, 20 uM, in 15 CV) to give 8.31 g of triazole (80%) as a white solid. NMR and mass m/z=2445 (M+Na) correspond with product.

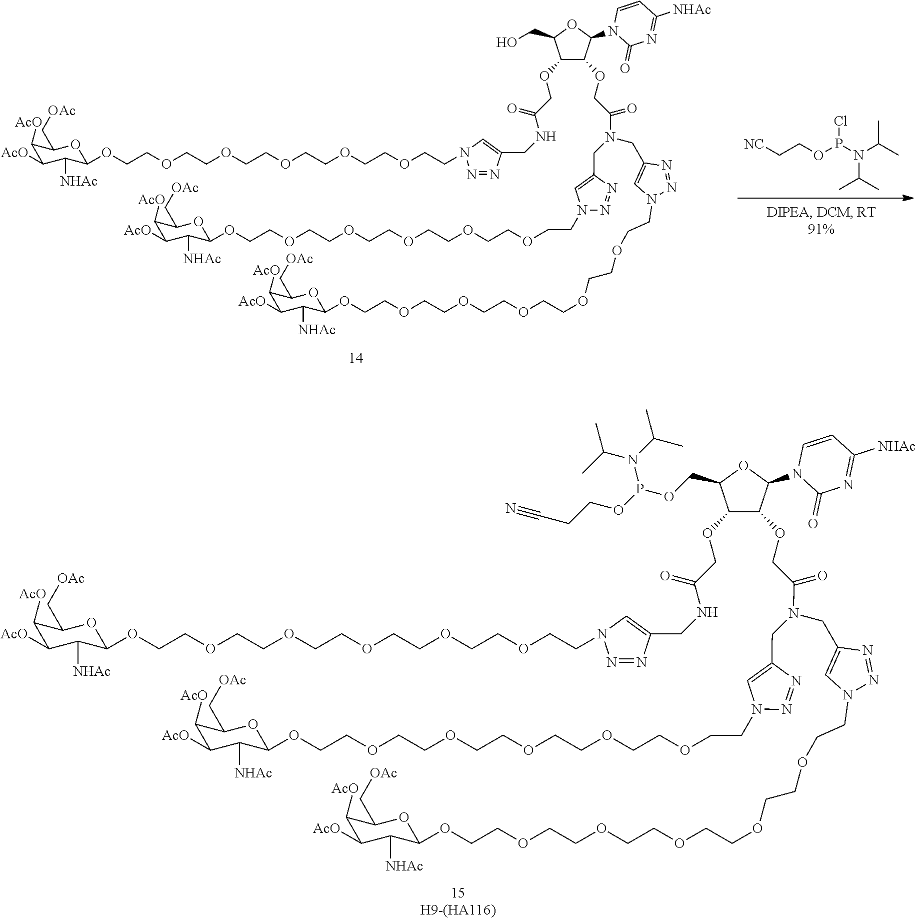

To a stirred solution of alcohol 14 (6.00 g, 2.474 mmol, 1 eq) and diisopropylethylamine (2.58 mL, 14.858 mmol, 6 eq), in DCM (50 mL), was added 2-cyanoethyl N, N-diisopropyl chlorophosphoramidite (1.65 mL 7.429 mmol, 3 eq) dropwise. The reaction mixture was stirred at room temperature for 1 h. The reaction mixture was quenched with Aq. Saturated NaHCO₃ solution (100 mL), extracted with DCM (500) ml, washed with brine (100 mL), and dried over N₂SO₄, and the evaporated crude product was loaded onto (pre-equilibrated two times with 1% Et₃N-DCM) Biotage silica gel column (100 g 20 μm) and purified by flash chromatography using 0-10% MeOH/DCM containing 1% Et₃N as an additive. Pure fractions were combined, concentrated, and dried under high vacuum to obtain Phosphoramidite 15 (6.9 g). NMR showed the triethylamine salt. The compound was dissolved in 150 ml DCM, washed with 2×50 mL aqueous saturated bicarbonate solution, and the organic layer was dried over Na₂SO₄, evaporated, and dried under high vacuum to obtain (5.9 g 91%) product as a white solid. 95% purity by HPLC, P31-NMR Mass (m/z 2644 M+Na), H1-NMR correspond with product.

### Example 16

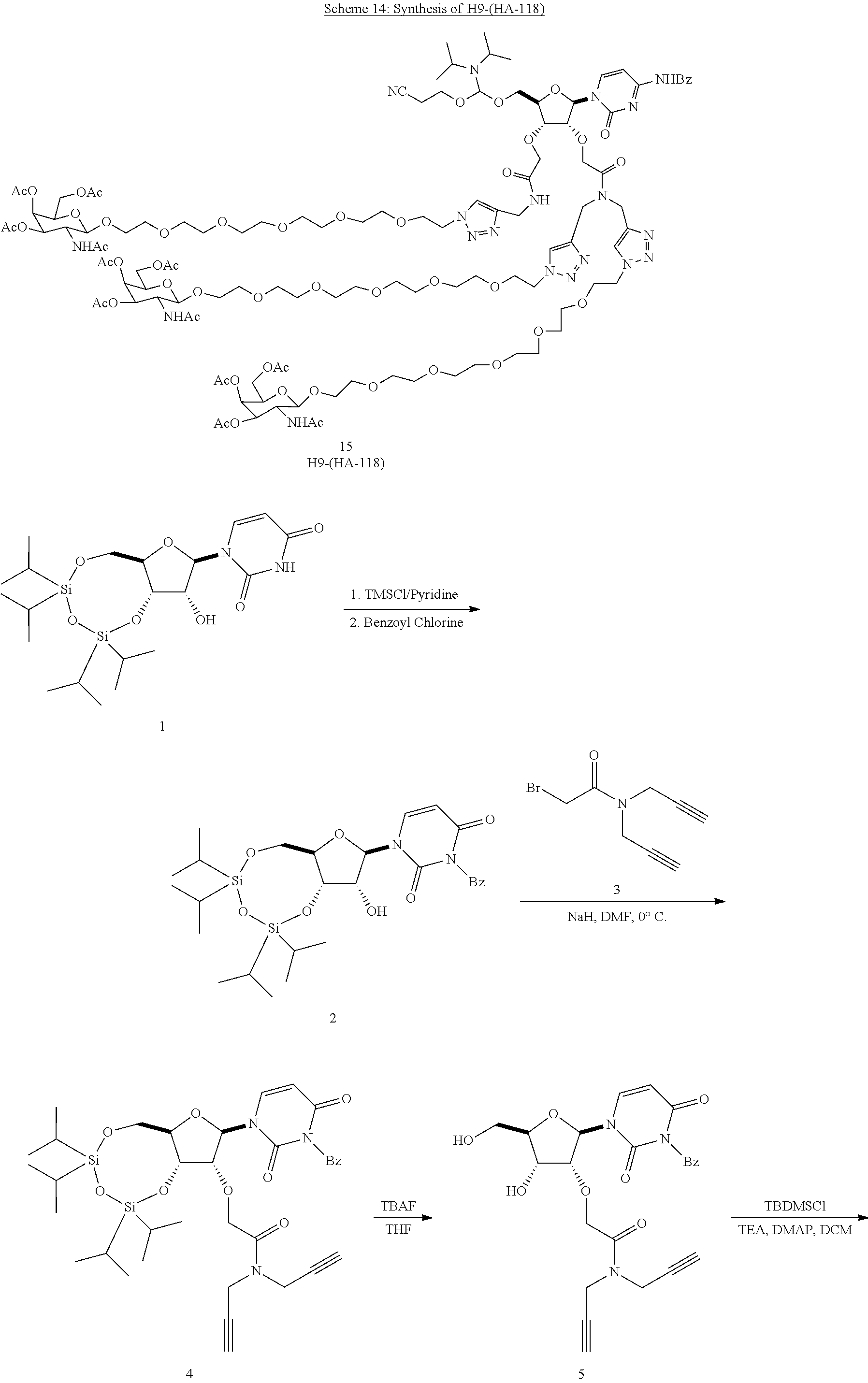

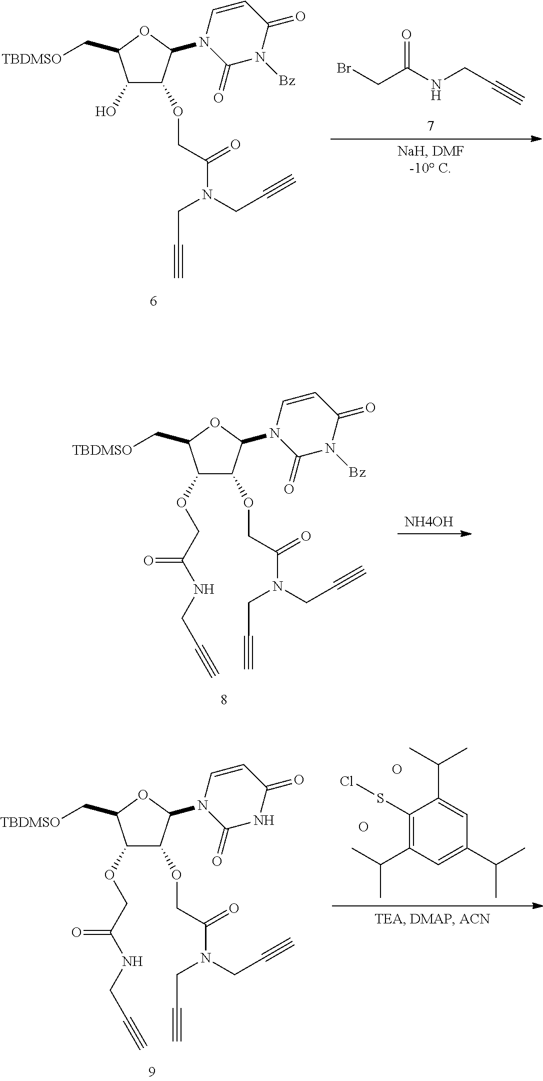

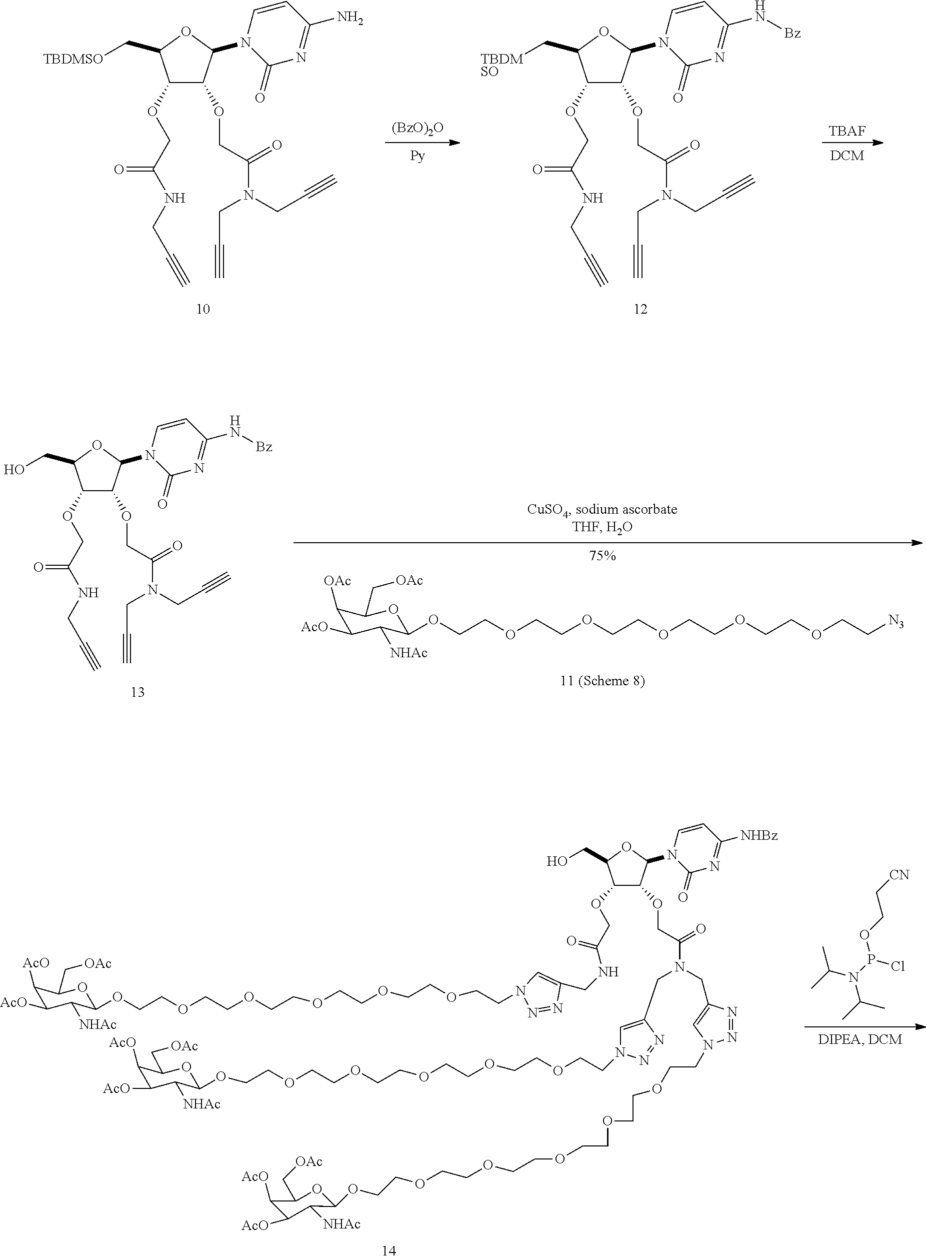

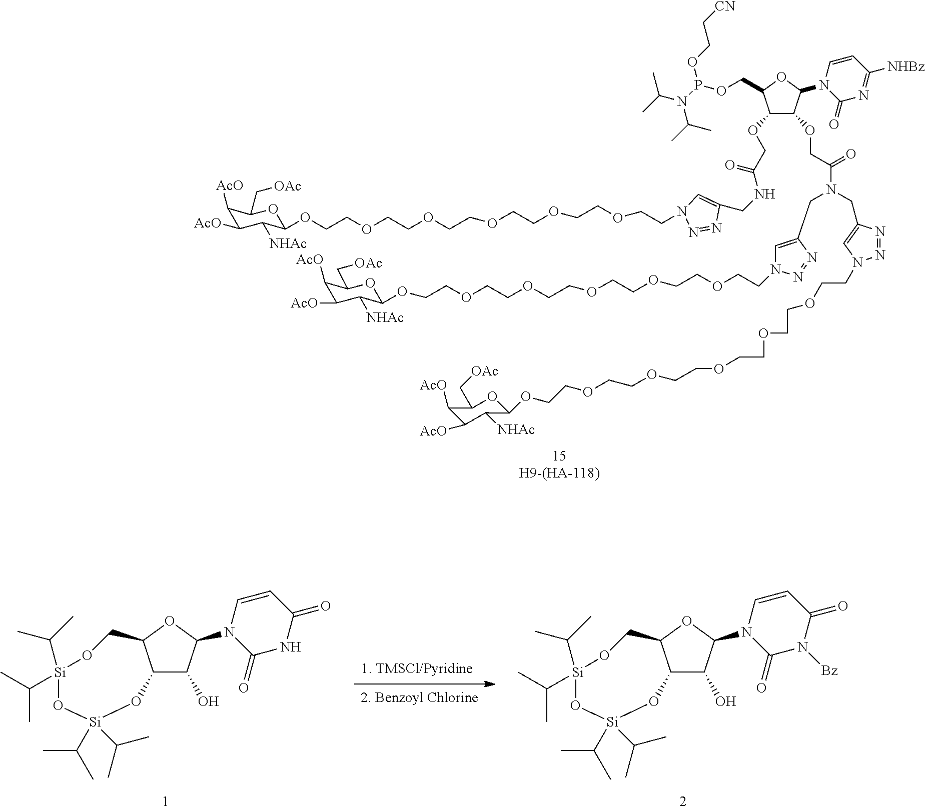

To a solution of SM 1 (5.0 g, 10.27 mmol, 1 eq) in anhydrous pyridine (40 mL) at RT was added chlorotrimethylsilane (3.129 mL, 24.655 mmol, 2.4 eq) dropwise. The mixture was stirred for 2 hours, then benzoyl chloride (2.862 mL, 24.655 mmol, 2.4 eq) was added dropwise to the reaction mixture. The reaction was stirred overnight at RT. H₂O (10 mL) was added to the reaction, and the reaction was stirred for 8 hours. The solvent was removed by rotavap. The crude mixture was partitioned between H₂O and EtOAc. The aqueous phase was extracted with EtOAc (2×100 mL). The combined organic layers were washed with brine, dried over Na₂SO₄, filtered, and concentrated. The residue was purified by 0-30% EtOAc in Hexanes to give 5.2 g product 2 (85%) as a white solid.

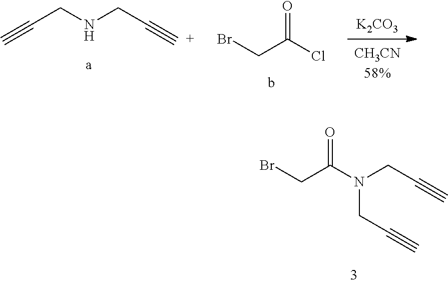

To a suspension of di-propargylamine (5 g, 53.688 mmol, 1 eq) and K₂CO₃ (37.1 g, 268.44 mmol, 5 eq) in CH₃CN (50 mL) at 0° C. was added bromoacetyl chloride (4.471 mL, 53.688 mmol, 1 eq). The reaction was stirred for 1 h at 0° C. LCMS showed the reaction was complete. The reaction was diluted with DCM and washed with water. The organic phase was dried over Na₂SO₄, concentrated, and purified by column chromatography (0%→30% EtOAc-hexanes) to afford 9 g product 3 (58%) as a yellow oil.

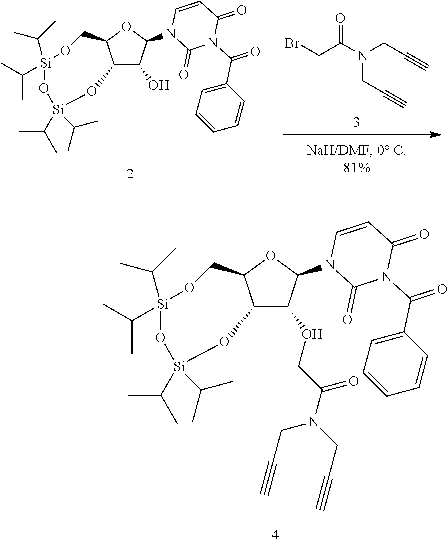

To a stirred solution of alcohol 2 (7.1 g, 12.017 mmol, 1 eq) in DMF (25 mL) at 0° C. was added amide 3 (3.86 g, 18.026 mmol, 1.5 eq) followed by NaH (0.625 g, 60% in mineral oil, 15.622 mmol, 1.3 eq) in two portions. The reaction was stirred at 0° C. for 45 min, then quenched by addition of ice water. Precipitated solids were filtered and redissolved in DCM, dried over Na₂SO₄, and concentrated. The crude was used as is for the next step.

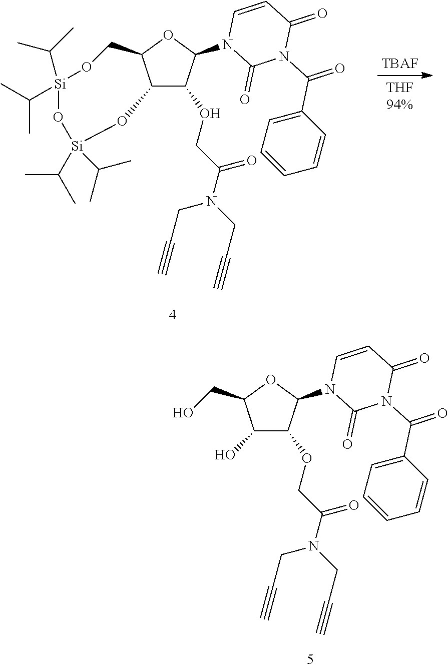

To a stirred solution of bis-alkyne 4 (7.2 g, 9.945 mmol, 1 eq) in THF (100 mL) was added TBAF (5.19 g, 19.917 mmol, 2 eq). The reaction was stirred 3 h at RT. The Reaction mixture was diluted with EtOAc and washed with NaHCO₃ and brine. The organic layer was dried over Na₂SO₄, filtered, and concentrated. The residue was purified by flash chromatography using 0 to 5% MeOH in EtOAc to give 4.5 g product 5 (79% over 2 steps) as a white solid.

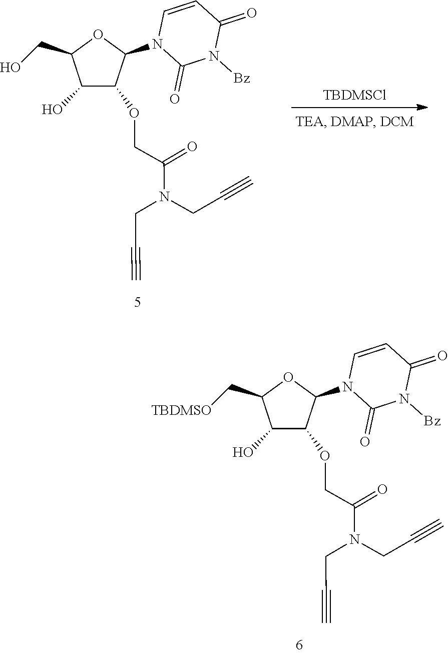

To a stirred solution of diol 5 (11 g, 18 mmol, 1.00 eq.) in DCM (100 mL) was added TEA (3.7 g, 36.9 mmol, 2 eq) and DMAP (0.27 g, 2.2 mmol, 0.1 eq) followed by the addition of TBDMS-Cl (3.3 g, 22.1 mmol, 1.2 eq). The reaction mixture was stirred at RT for 12 h. LCMS showed complete reaction. To the reaction mixture was added water (50 mL), and the DCM layer was separated, dried over Na₂SO₄, filtered, and concentrated. The residue was purified by column chromatography on silica gel using 0-60% Hexane/EA as an eluent, and pure fractions were combined and concentrated to obtain product as a white solid (11.5 g; 84%). Product was confirmed by LCMS.

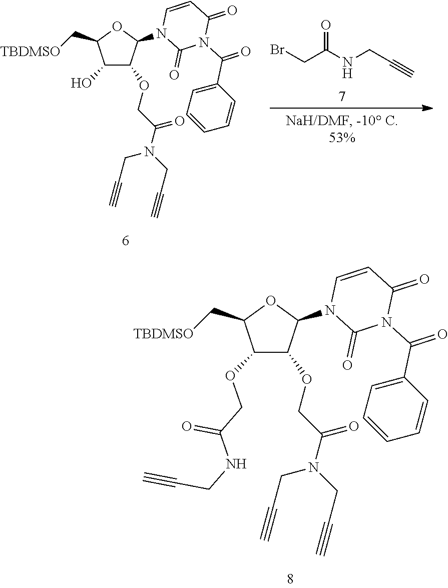

To a stirred mixture of alcohol 6 (11.5 g, 19.3 mmol, 1 eq) and bromo compound 7 (4.4 g, 23.19 mmol, 1.3 eq) in THF was added NaH (0.55 g, 23.19 mmol, 1.2 eq) at −10° C. The RM was stirred for 1 h at −10° C. After completion, the reaction was quenched with water and extracted with EA, dried over Na₂SO₄, and filtered and concentrated under reduced pressure. The resulting crude was purified by Biotage column chromatography (0-60% ethyl acetate/hexanes) to give compound 8 as a beige solid (7 g, 52%). LCMS and ¹H-NMR correspond with product.

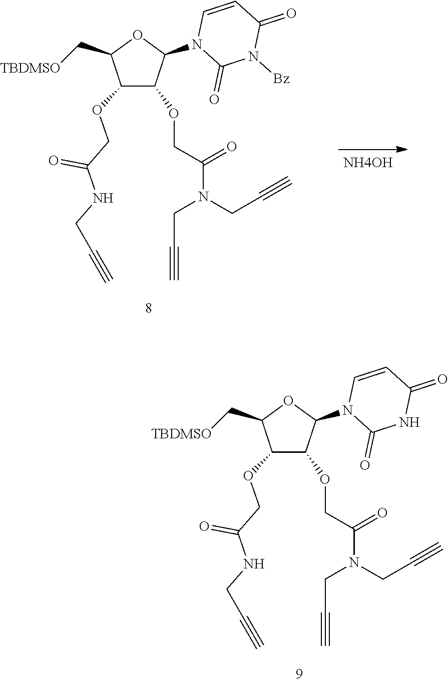

Aqueous NH₄OH solution (50 ml) was added to a solution of compound 8 (6.8 g, 9.8 mmol, 1 eq) in THF (50 mL), and the mixture was stirred for 3 h at room temperature. After completion of the reaction, water was added and extracted with DCM, dried over Na₂SO₄, filtered, and concentrated under reduced pressure. The resulting crude was purified by Biotage column chromatography (1:10 DCM/MeOH) to give 3 as a beige solid (4 g, 70%).

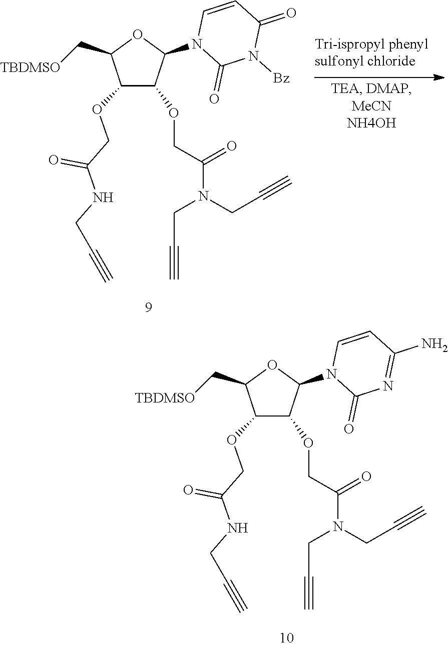

To a stirred solution of the uridine derivative 9 (4.5 g, 7.143 mmol, 1 eq) in anhydrous CH₃CN (50 mL) was added DMAP (462 mg, 3.784 mmol, 2 eq), triethylamine (0.527 mL, 3.784 mmol, 2 eq), and TiPSCl (1.146 g, 2.3.784 mmol, 2 eq). The reaction was stirred at rt for 2 h. Concentrated ammonium hydroxide (50% v/v in H₂O) (10 mL) was added to the reaction. The reaction was stirred overnight. The crude reaction was concentrated under reduced pressure. The residue was partitioned between DCM and sat. NH₄Cl. The aqueous layer was extracted with DCM (1×100 mL). The organic layers were collected, dried over Na₂SO₄, filtered, and concentrated. The residue was purified by Biotage column (25 g, 20 uM) using 0-5% MeOH in DCM in 10 CV to 3.4 g of cytosine (91%) as an off-white solid.

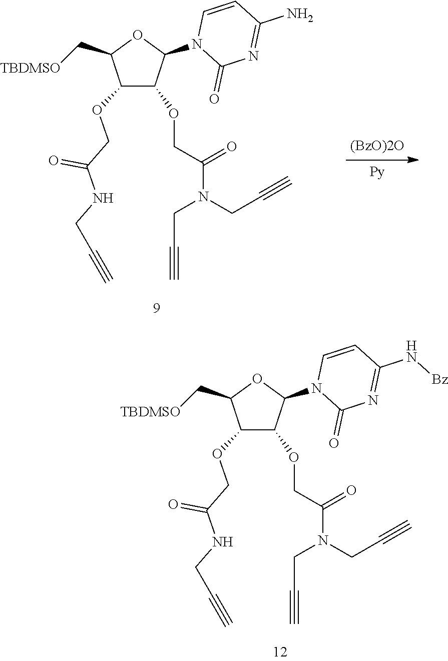

Compound 9 (4 g, 6.83 mmol, 1 eq) and benzoic anhydride (3.09 g, 13.67 mmol, 2 eq) were dissolved in 50 mL of dry DMF, and the reaction mixture was stirred for 24 h at 25° C. Py (2 ml) was then added, and the reaction was stirred another 12 h at RT. After completion of the reaction, the excess DMF was removed under reduced pressure and the crude product was purified by using silica gel column chromatography (0-100% Ethylacetate/Hexane) to obtain compound 12 as a beige solid (2.8 g 77%).

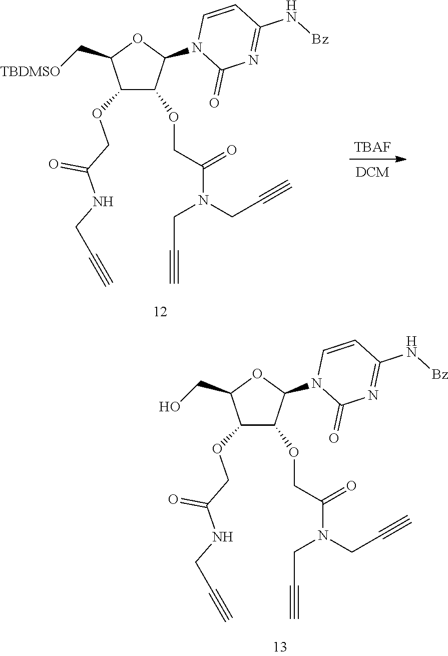

To a stirred solution of TBS-ether 12 (1.4 g, 2.031 mmol, 1 eq) in THF (30 mL) was added TBAF (1 g, 4.063 mmol, 2 eq). The reaction was stirred for 3 h at RT. The reaction mixture was diluted with EtOAc and washed with NaHCO₃ and brine. The organic layer was dried over Na₂SO₄, filtered, and concentrated. The residue was purified by flash chromatography using 0 to 10% MeOH in EtOAc to give 0.8 g product 13 (70%) as a white solid.

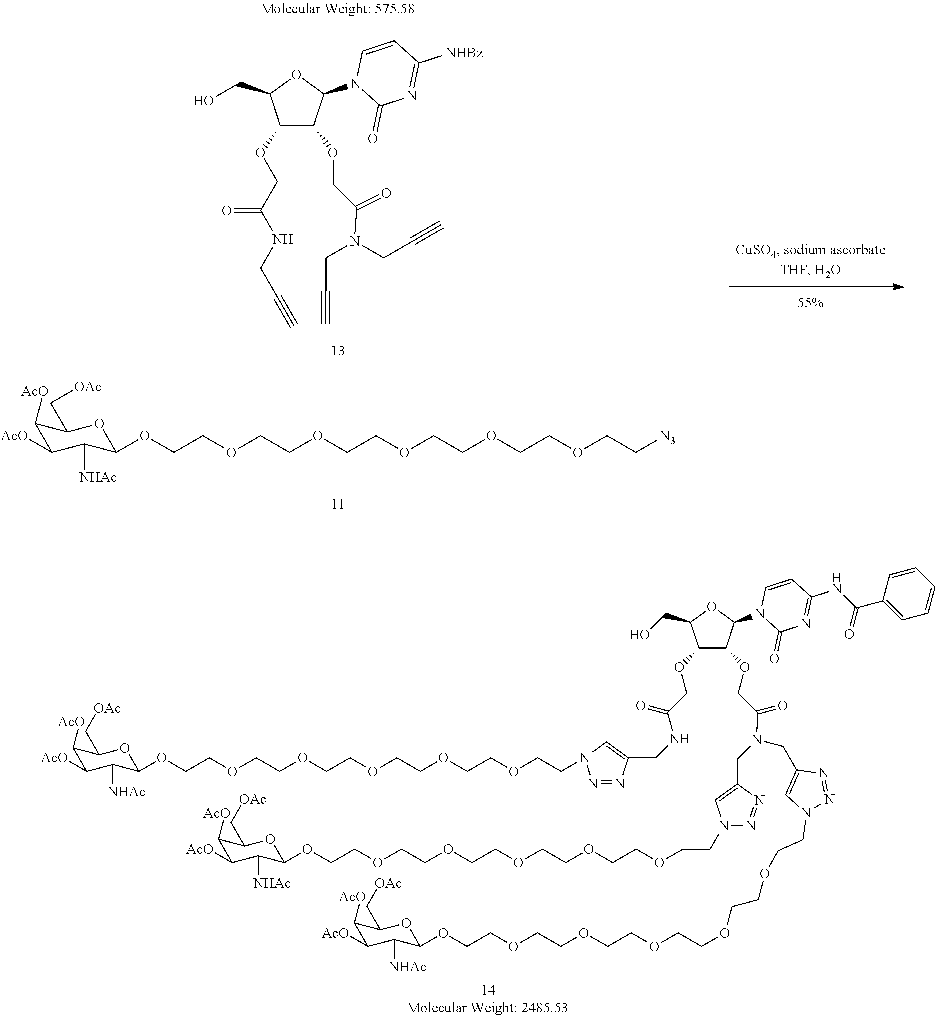

To a stirred solution of alkyne 13 (0.8 g, 1.39 mmol, 1 eq) and azide 11 (2.83 g, 4.45 mmol, 3.2 eq) in THF (40 mL) were added CuSO₄·5H₂O (0.17 g, 0.64 mmol, 0.5 eq) in water (5 mL) followed by sodium ascorbate (0.2 g, 1.04 mmol, 0.75 eq) in water (5 mL). The mixture was stirred for 3 h at room temperature. LCMS showed complete product formation and disappearance of alkyne. The reaction mixture was diluted with aq. Saturated NaHCO₃ (50 mL) and extracted with DCM (2×50 mL). The organic layers were combined and washed with brine solution (100 mL), dried over Na₂SO₄, and concentrated, and the resulting crude was purified by column chromatography using 0-30% MeOH/DCM as an eluent. Pure fractions were combined and concentrated, and the resulting solids were co-evaporated with toluene and dried under high vacuum to obtain tris-triazole (2.7 g 78%) as a white solid. LCMS and NMR correspond with the product.

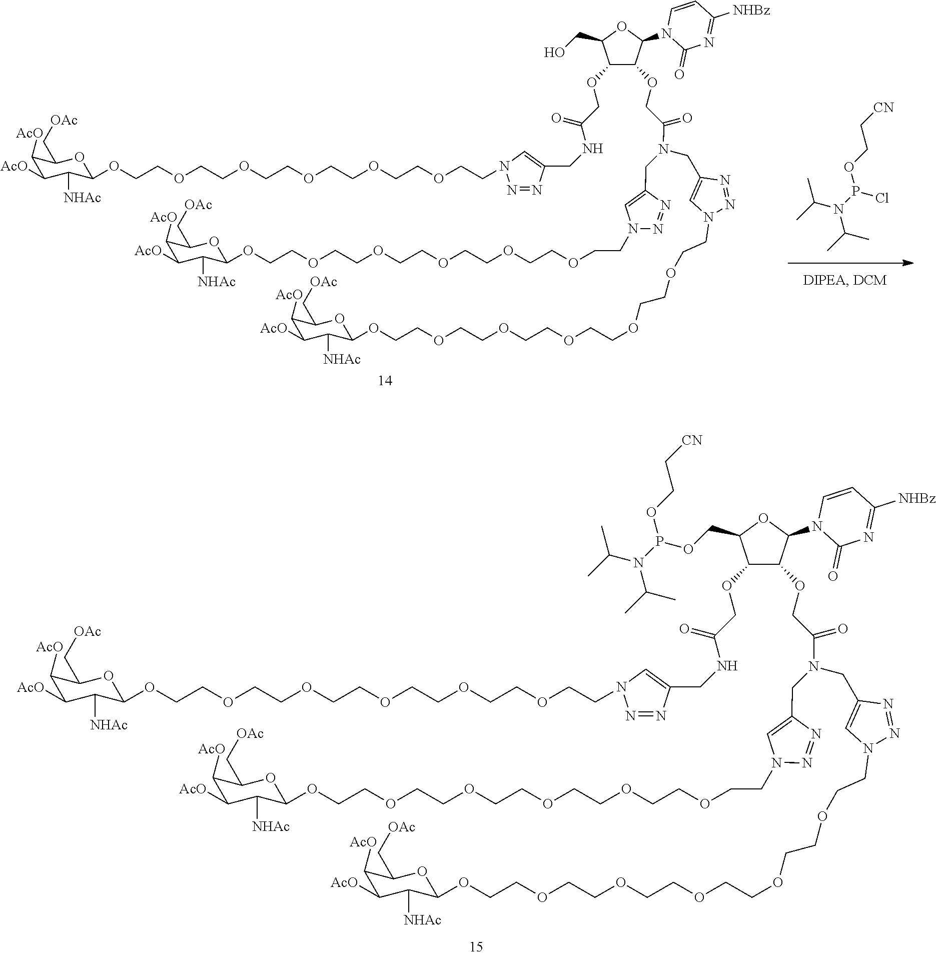

To a stirred solution of alcohol 14 (1.8 g, 0.724 mmol, 1 eq) and diisopropylethylamine (0.56 g, 4.346 mmol, 6 eq), in DCM (20 mL), was added 2-cyanoethyl N,N-diisopropyl chlorophosphoramidite (0.51 g, 2.173 mmol, 3 eq) dropwise. The reaction mixture was stirred at room temperature for 30 min. LCMS showed completion of the reaction. The reaction mixture was quenched with Aq. Saturated NaHCO₃ solution (20 mL), extracted with DCM (2×30 mL), washed with brine (20 mL), dried over N₂SO₄, and the evaporated crude product was loaded onto pre-equilibrated (1% Et₃N-MeOH:1% DCM (1:10) Biotage silica gel column (50 g 20 μm) and purified by flash chromatography using 0-10% MeOH/DCM containing 1% Et₃N as an additive. Pure fractions were collected and concentrated to obtain 1.5 g amidite 15 (80%) with >95% HPLC purity. NMR and mass correspond with the product.

### Example 17

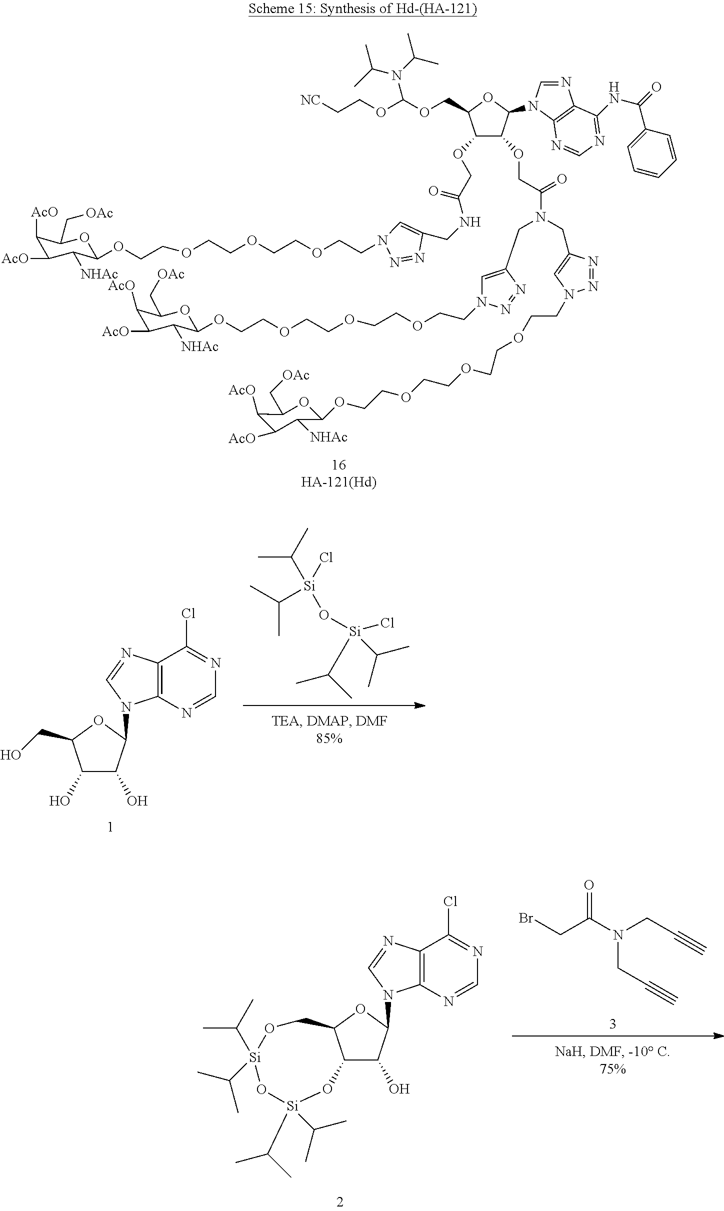

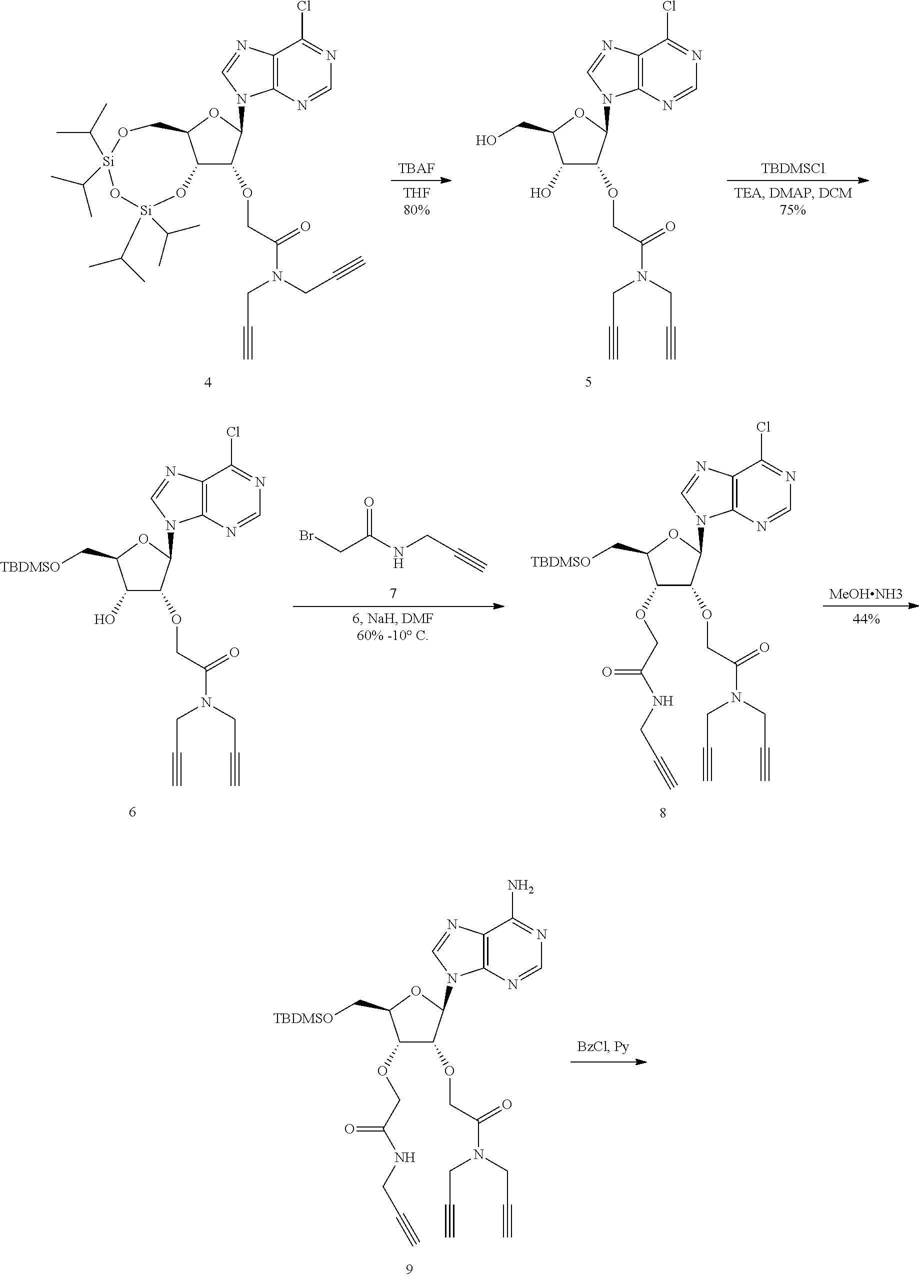

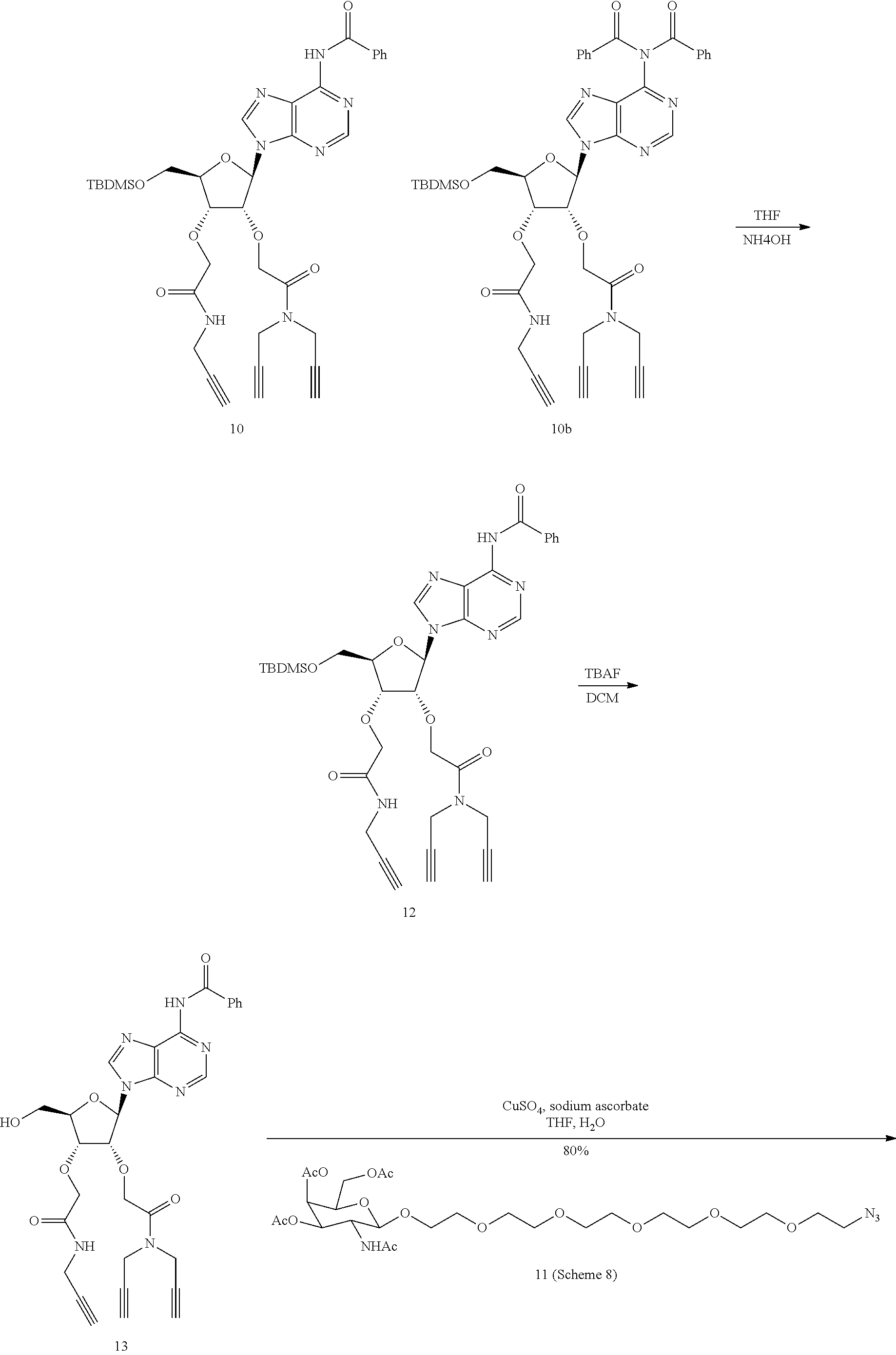

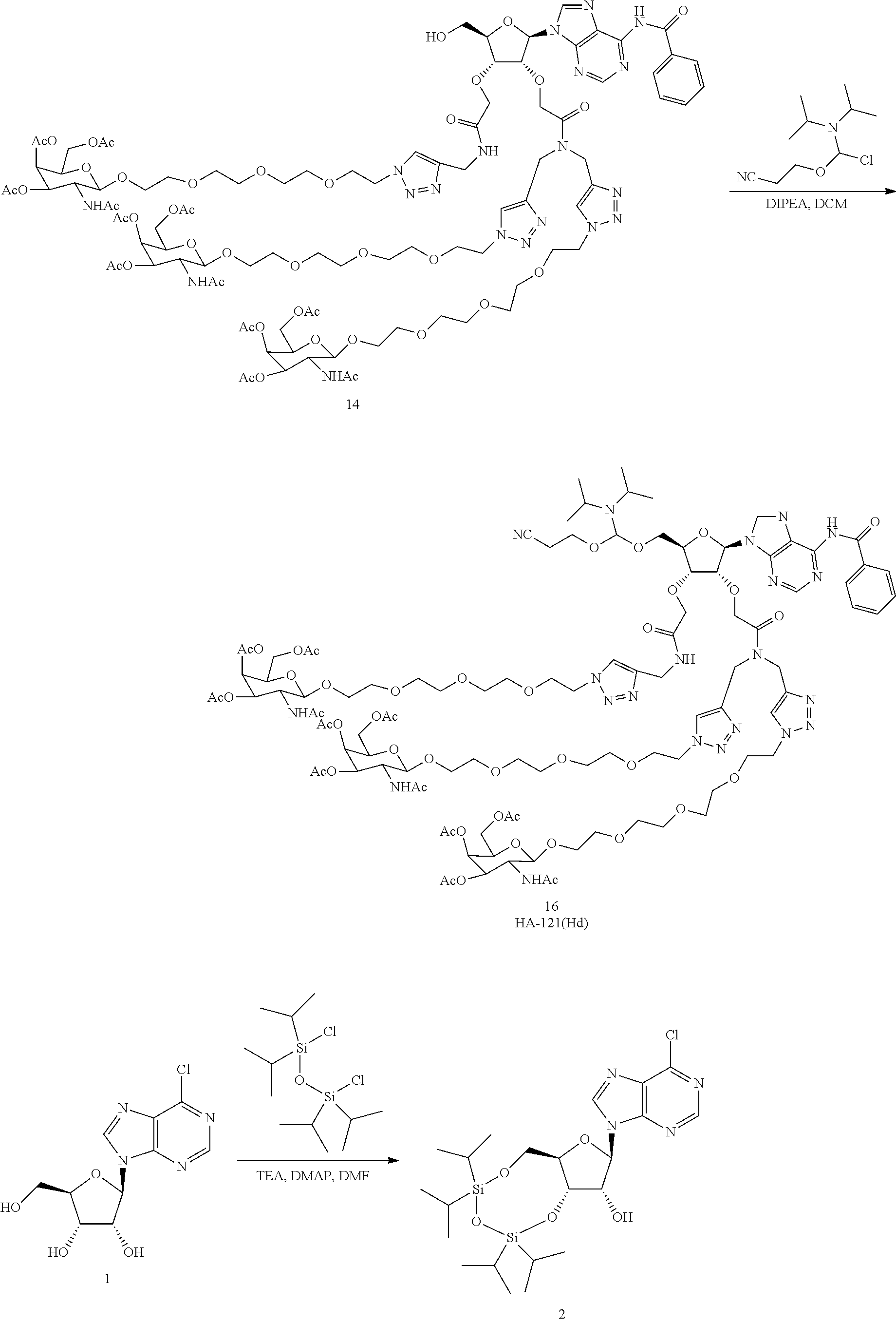

6-Chloropurine riboside (5 g, 17.483 mmol) and 1,3-dichloro-1,1,3,3-tetraisopropyldisiloxane compound (6.6 g, 20.978 mmol) were dissolved in anhydrous DMF (50 mL). To the reaction mixture was added TEA (7.25 mL, 52.447 mmol), followed by DMAP (0.2 g, 1.75 mmol). The reaction mixture was stirred for 6 h at RT. The reaction mixture was then poured into water (100 mL), and the resulting suspension was extracted with dichloromethane (3×50 mL). The combined organic extracts were washed with brine (1×20 mL), dried over magnesium sulfate, and concentrated under reduced pressure. The resulting crude was purified by Biotage column chromatography (0-60% Hexane/Ethyl acetate) to give compound 2 (8 g, 86%) as a white solid.

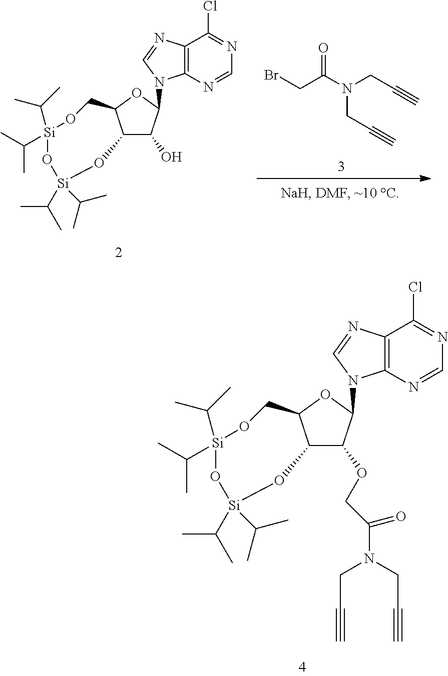

To a stirred mixture of 2 (5 g, 9.452 mmol) and bromo compound 3 (2.42 g, 11.342 mmol) in DMF (50 mL) was added NaH (0.272 g, 11.342 mmol) at −10° C. The RM was stirred for 1 hour at −10° C. After completion of the reaction, ice cold water was added to the RM. Precipitated solid was filtered and redissolved in DCM, dried with Na₂SO₄, filtered, and concentrated. The crude product was was used for the next step without further purification.

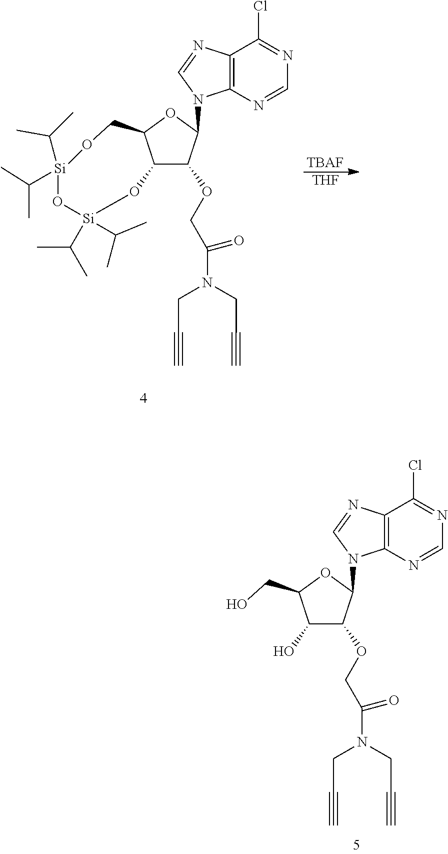

To a stirred solution of di-silyl ether 4 (4 g, 6.042 mmol) in THF (50 mL) was added TBAF (3.13 g, 12.085 mmol). The reaction mixture was stirred at RT for 3 h. LCMS showed complete deprotection. Water was added to the reaction mixture (50 mL), extracted with DCM (2×50 mL), dried over Na₂SO₄, filtered, and concentrated. The residue was purified by column chromatography on silica gel using 0-10% DCM/MeOH as an eluent, and pure fractions were combined and concentrated to obtain diol 5 (2 g, 80%) as a white solid.

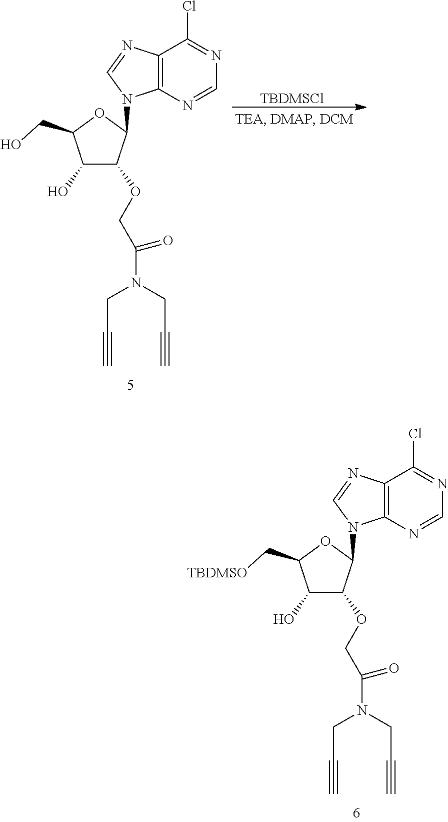

To a stirred solution of diol 5 (2 g, 4.773 mmol) in DCM (20 mL) was added TEA (0.96, 9.54 mmol) and DMAP (0.1 g) followed by the addition of TBDMSCl (0.86 g, 5.728 mmol). The reaction mixture was stirred at RT for 12 h. LCMS showed that the reaction was complete. The reaction mixture was quenched with water (50 mL), extracted with DCM (2×40 mL), dried over Na₂SO₄, filtered, and concentrated. The residue was purified by column chromatography on silica gel using 0-60% Hexane/EA as an eluent, and pure fractions were combined and concentrated to obtain product 6 (1.8 g, 75%) as a white solid.

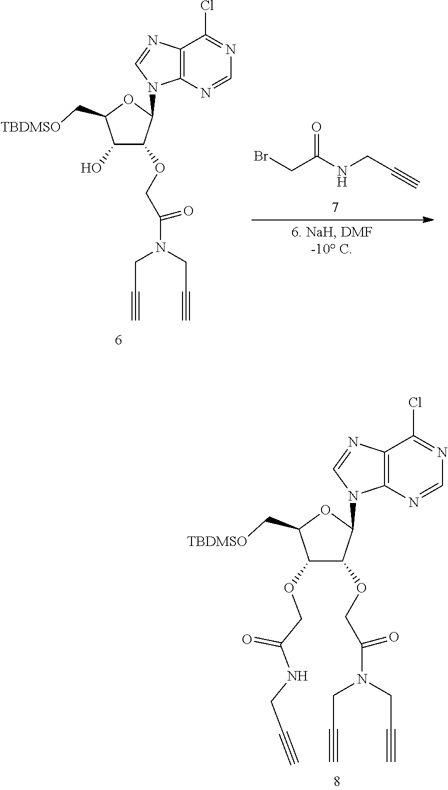

To a stirred mixture of alcohol 6 (1.8 g, 3.371 mmol) and bromo compound 7 (0.71 g, 4.045 mmol) in DMF (20 mL) was added NaH (0.097 g, 4.045 mmol) at −10° C. The RM was stirred for 1 h at −10° C. After completion, the reaction was quenched with water, extracted with EA, washed with brine, dried over Na₂SO₄, filtered, and concentrated under reduced pressure. The resulting crude was purified by Biotage column chromatography (0-60 Hexane/ethyl acetate) to obtain product 8 (1.3 g 60%) as a white solid.

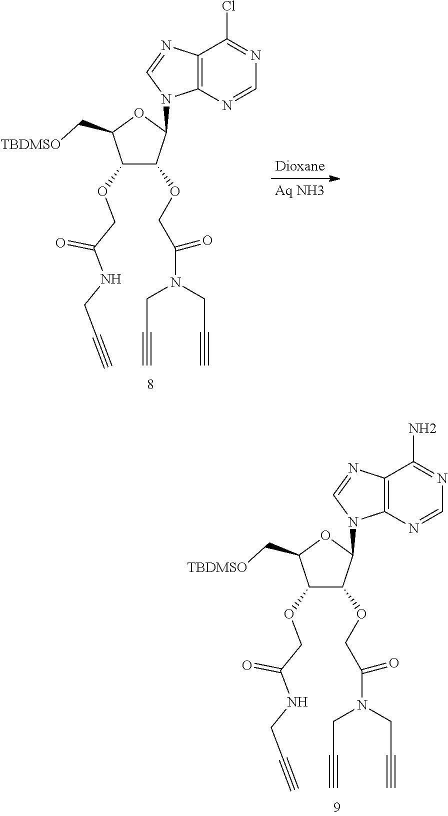 [SMILES: C#CCNC(=O)CO[C@H]1[C@@H](OCC(=O)N(CC#C)CC#C)[C@H](n2cnc3c(Cl)ncnc32)O[C@@H]1CO[Si](C)(C)C(C)(C)C ; C#CCNC(=O)CO[C@H]1[C@@H](OCC(=O)N(CC#C)CC#C)[C@H](n2cnc3c(N)ncnc32)O[C@@H]1CO[Si](C)(C)C(C)(C)C]

A mixture of 8 (0.1 g, 0.159 mmol), aq. NH₃ (2 mL), and dioxane (5 mL) in a sealed tube was heated for 12 h at 70° C. After completion of the reaction, the excess of dioxane and water was removed under rotavap. The resulting crude was purified by Biotage column chromatography using 0-100 Ethyl acetate to give (0.075 g, 70%) as a beige solid.

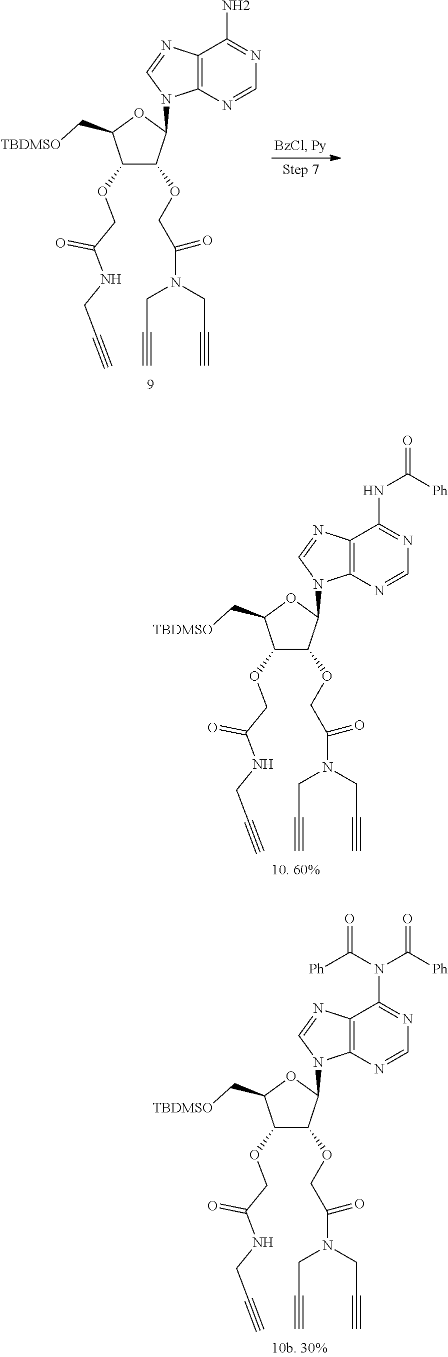 [SMILES: C#CCNC(=O)CO[C@H]1[C@@H](OCC(=O)N(CC#C)CC#C)[C@H](n2cnc3c(N)ncnc32)O[C@@H]1CO[Si](C)(C)C(C)(C)C ; C#CCNC(=O)CO[C@H]1[C@@H](OCC(=O)N(CC#C)CC#C)[C@H](n2cnc3c(NC(=O)c4ccccc4)ncnc32)O[C@@H]1CO[Si](C)(C)C(C)(C)C ; C#CCNC(=O)CO[C@H]1[C@@H](OCC(=O)N(CC#C)CC#C)[C@H](n2cnc3c(N(C(=O)c4ccccc4)C(=O)c4ccccc4)ncnc32)O[C@@H]1CO[Si](C)(C)C(C)(C)C]

To a stirred solution of amine 9 (0.1 g, 0.164 mmol) in Py (5 mL) was added BzCl (0.023 mL, 0.328 mmol). The RM was stirred for 2 h at 0° C. After completion of the reaction, 2N HCl was added and extracted with DCM, and the organic layer was separated and dried over Na₂SO₄, filtered, and concentrated under rotavap. The resulting crude mixture of 10 & 10b was used as is for the next step.

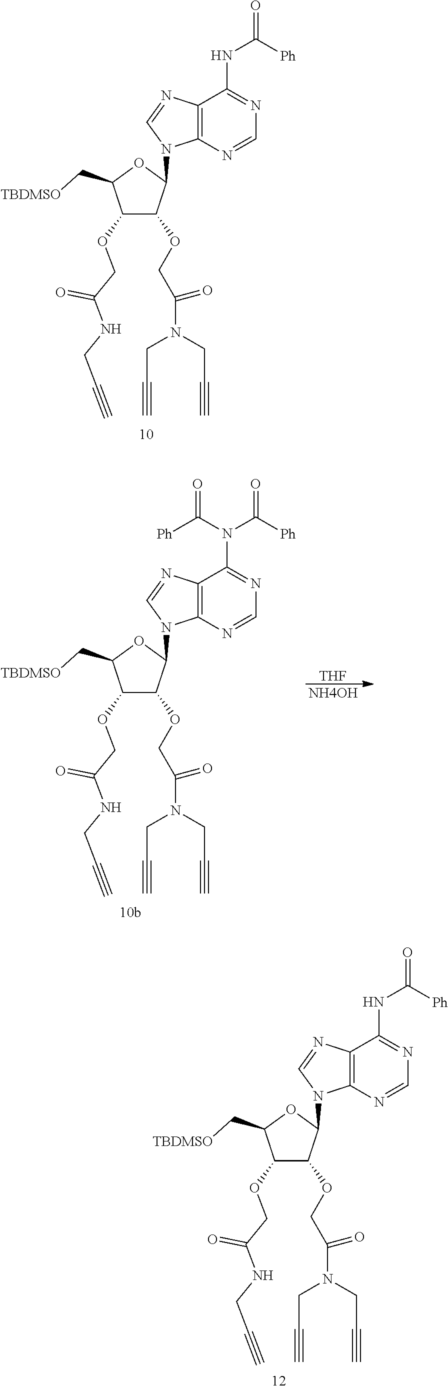 [SMILES: C#CCNC(=O)CO[C@H]1[C@@H](OCC(=O)N(CC#C)CC#C)[C@H](n2cnc3c(NC(=O)c4ccccc4)ncnc32)O[C@@H]1CO[Si](C)(C)C(C)(C)C ; C#CCNC(=O)CO[C@H]1[C@@H](OCC(=O)N(CC#C)CC#C)[C@H](n2cnc3c(N(C(=O)c4ccccc4)C(=O)c4ccccc4)ncnc32)O[C@@H]1CO[Si](C)(C)C(C)(C)C ; "NH4OH" ; "THF" ; C#CCNC(=O)CO[C@H]1[C@@H](OCC(=O)N(CC#C)CC#C)[C@H](n2cnc3c(NC(=O)c4ccccc4)ncnc32)O[C@@H]1CO[Si](C)(C)C(C)(C)C]

To a stirred solution of crude 10 & 10b (0.15 g) in THF (5 mL) was added NH₄OH (2 mL). The RM was stirred for 3 h at 0° C. After completion of the reaction, to the RM was added 2N HCl and extracted with DCM, and the organic layer was separated and dried with Na₂SO₄, filtered, and concentrated under rotavap. The resulting crude mixture was used for the next step without any purification.

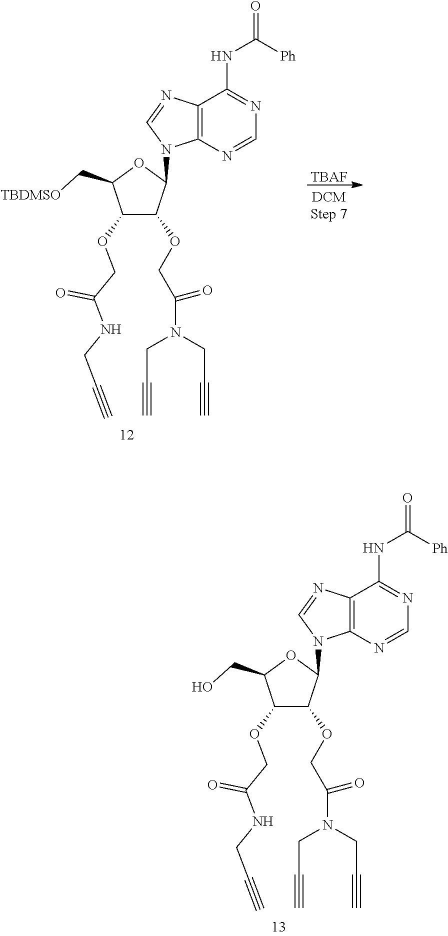

To a stirred solution of crude compound 12 (0.12 g) in THF (10 mL) was added TBAF (0.1 g). The reaction mixture was stirred at RT for 3 h. LCMS showed complete deprotection. To the reaction mixture was added water (30 mL), and then the reaction mixture was extracted with DCM (2×30 mL), dried over Na₂SO₄, filtered, and concentrated. The residue was purified by column chromatography on silica gel using 0-10% DCM/MeOH as an eluent, and pure fractions were combined and concentrated to obtain (0.06 g 43%) as a white solid. Product was confirmed by LCMS and NMR.

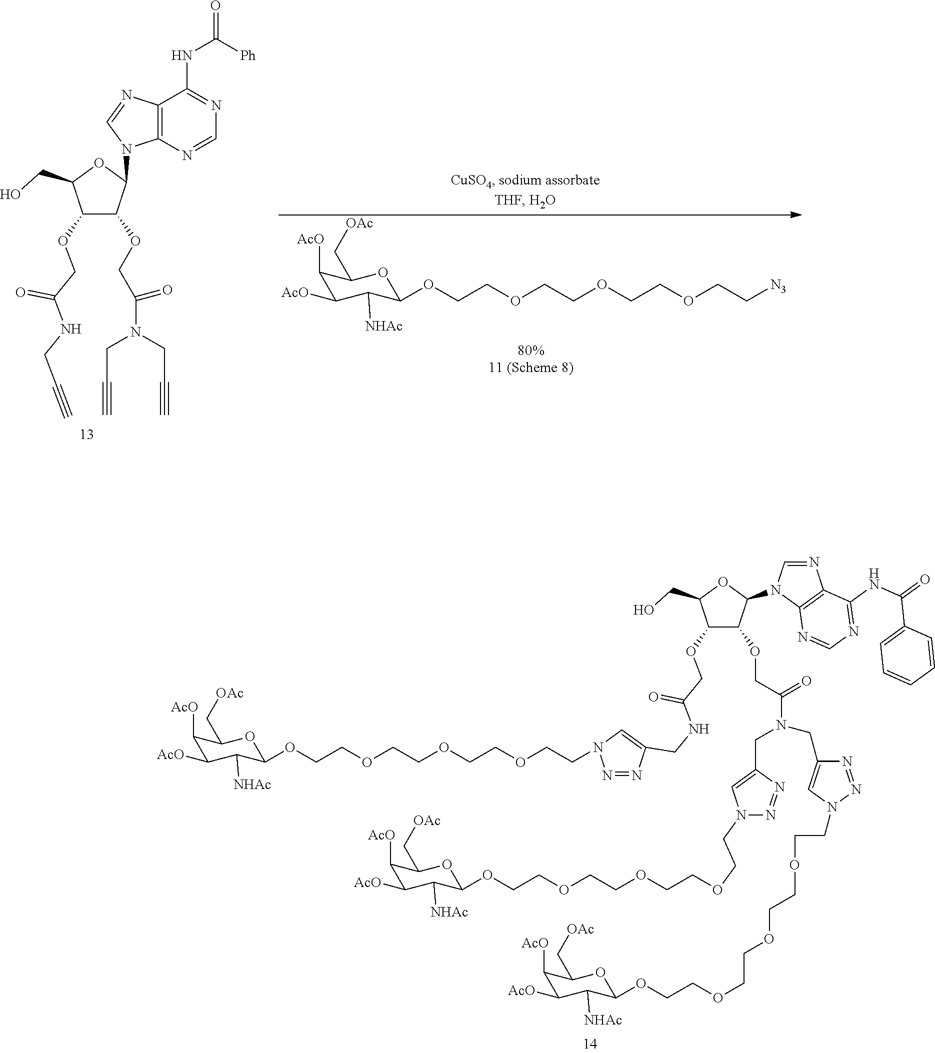 [SMILES: C#CCNC(=O)CO[C@H]1[C@@H](OCC(=O)N(CC#C)CC#C)[C@H](n2cnc3c(NC(=O)c4ccccc4)ncnc32)O[C@@H]1CO ; CC(=O)NC1C(OCCOCCOCCOCCN=[N+]=[N-])OC(COC(C)=O)C(OC(C)=O)C1OC(C)=O ; CC(=O)NC1C(OCCOCCOCCOCCn2cc(CNC(=O)CO[C@H]3[C@@H](OCC(=O)N(Cc4cn(CCOCCOCCOCCOC5OC(COC(C)=O)C(OC(C)=O)C(OC(C)=O)C5NC(C)=O)nn4)Cc4cn(CCOCCOCCOCCOC5OC(COC(C)=O)C(OC(C)=O)C(OC(C)=O)C5NC(C)=O)nn4)[C@H](n4cnc5c(NC(=O)c6ccccc6)ncnc54)O[C@@H]3CO)nn2)OC(COC(C)=O)C(OC(C)=O)C1OC(C)=O]

To a stirred solution of alkyne 13 (1.35 g, 2.25 mmol) and azide 11 (3.93 g, 7.21 mmol) in THF (50 mL), were added CuSO₄·5H₂O (0.28 g, 1.12 mmol), and Na-ascorbate (0.32 g, 1.62 mmol) in water (10+10 mL), and the mixture was stirred for 3 h at room temperature. LCMS showed product formation and complete disappearance alkyne. The reaction mixture was diluted with aq. Saturated NaHCO₃ (100 mL) and extracted with DCM 2×100 mL, and the organic layer was washed with brine solution (100 mL). The combined extracts were dried over Na₂SO₄ and concentrated, and the resulting crude was purified by column chromatography using 0-50% MeOH/DCM as an eluent. Pure fractions were combined and concentrated, and the resulting solids were co-evaporated with toluene and dried under high vacuum to obtain tris-triazole (4 g, 82%) as a white solid.

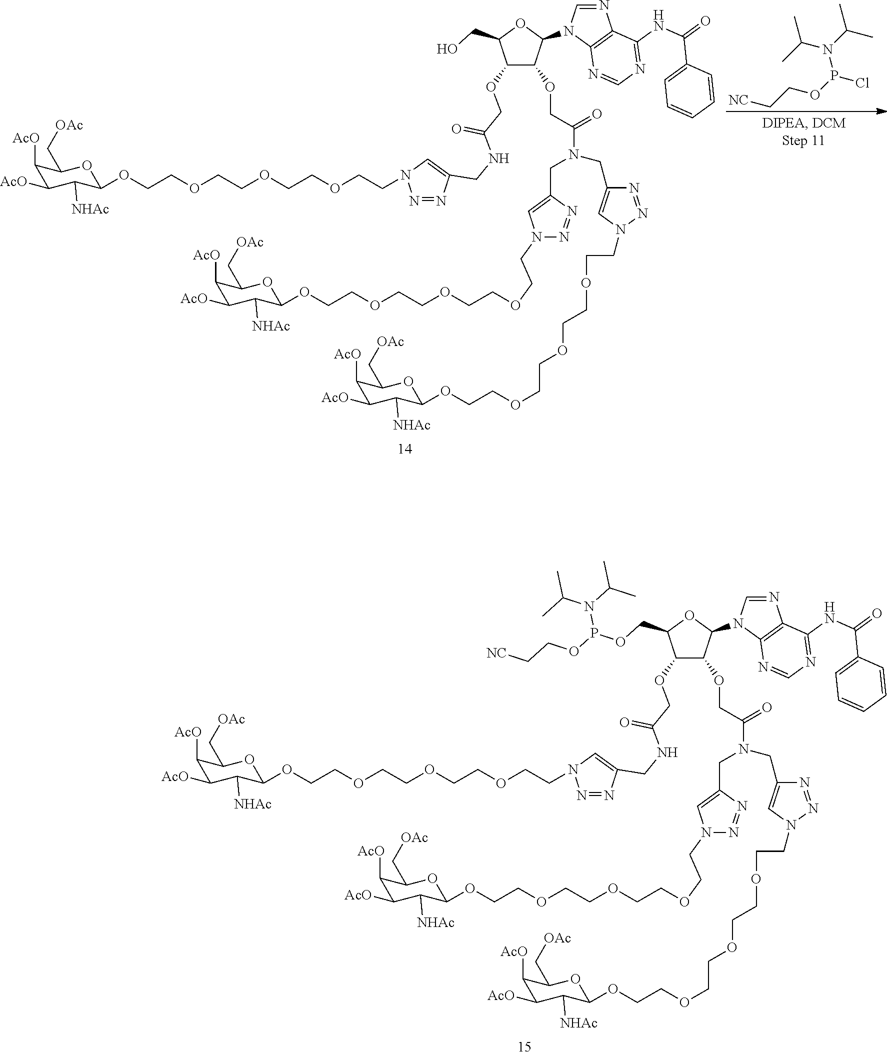

To a stirred solution of alcohol 14 (0.68 g, 0.688 mmol) and diisopropylethylamine (0.51 g, 4.008 mmol), in DCM (10 mL), was added 2-cyanoethyl N,N-diisopropyl chlorophosphoramidite (0.47 g, 2.004 mmol) dropwise. The reaction mixture was stirred at room temperature for 15 min. LCMS showed completion of the reaction. The reaction mixture was quenched with Aq. Saturated NaHCO₃ solution (20 mL), extracted with DCM (2×30) ml, washed with brine (20 mL), dried over Na₂SO₄, and evaporated. The crude product was then loaded on to pre-equilibrated (1% Et₃N-MeOH:1% DCM (1:20) biotage silica gel column (50 g 20 μm) and purified by flash chromatography using 0-30% MeOH/DCM containing 1% Et₃N as an additive. Pure fractions were collected and concentrated to obtain amidite 15 (1.6 g 74%) as an off-white solid with >95% HPLC purity. Mass and NMR correspond with the product.

### Example 18

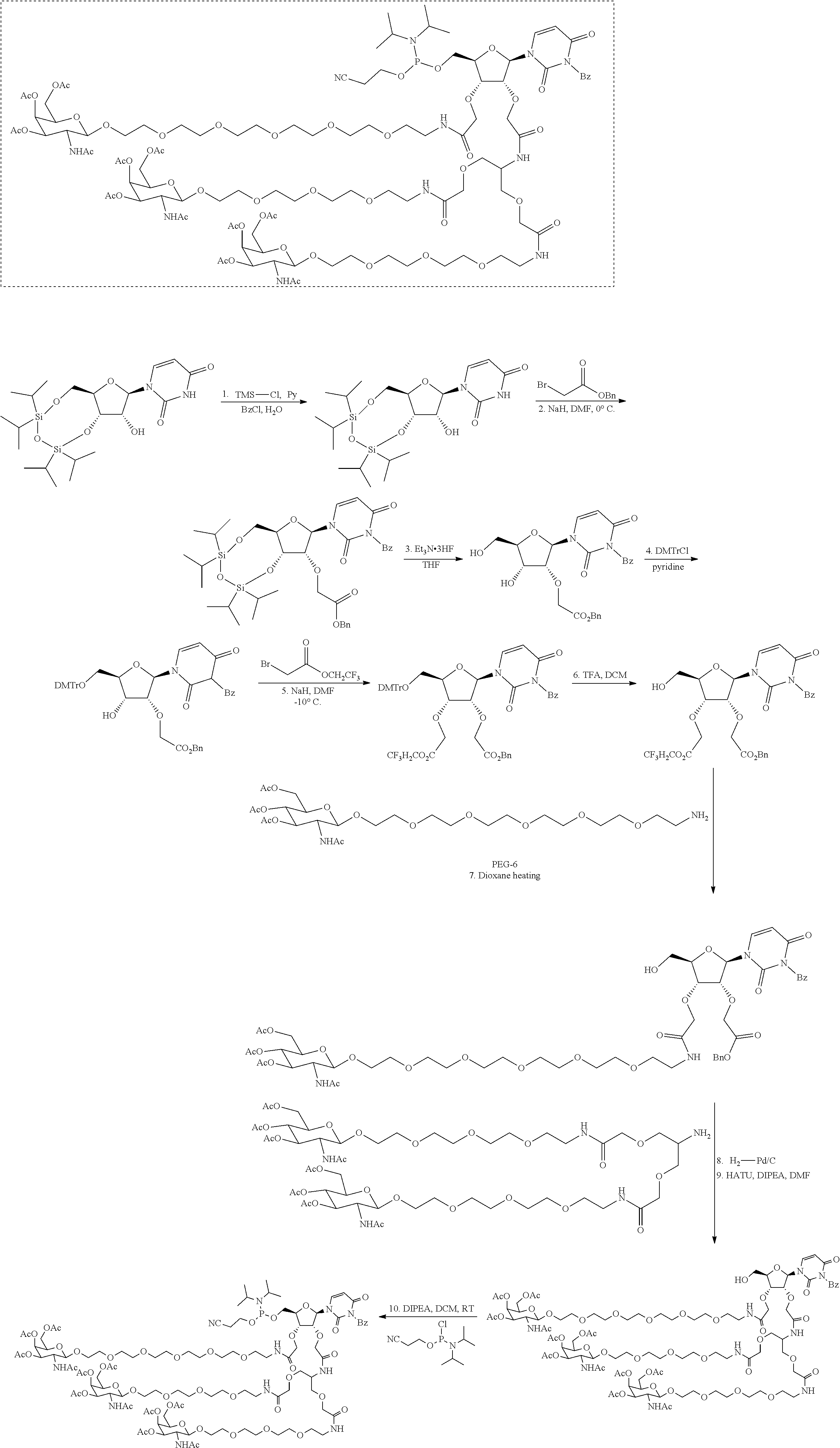

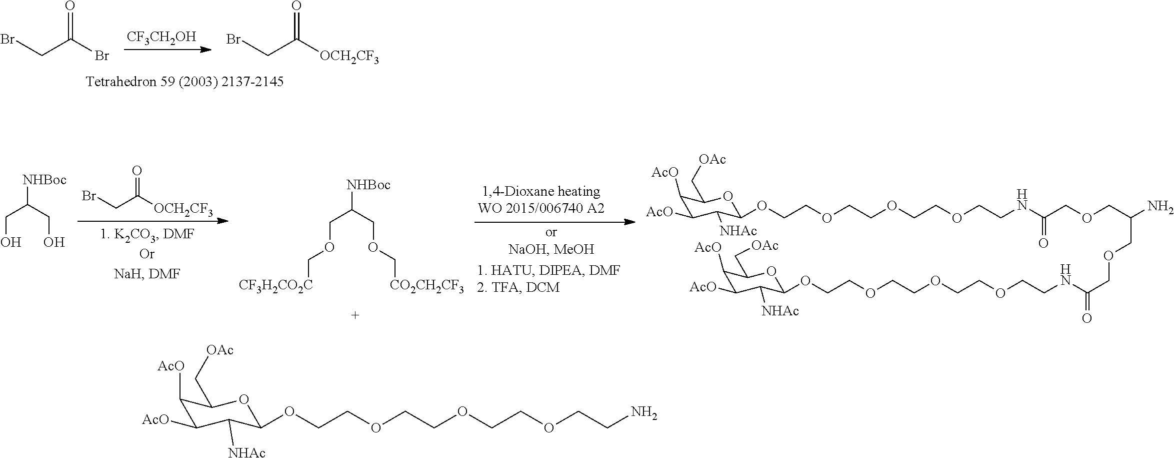

### Example 19

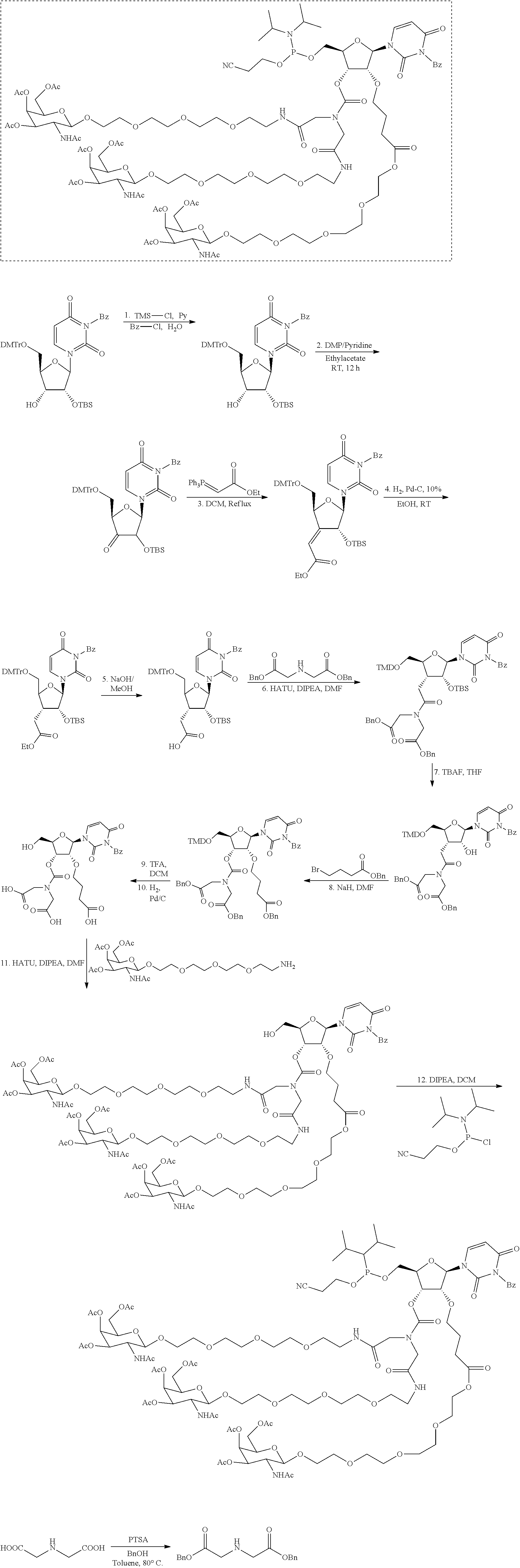

### Example 20

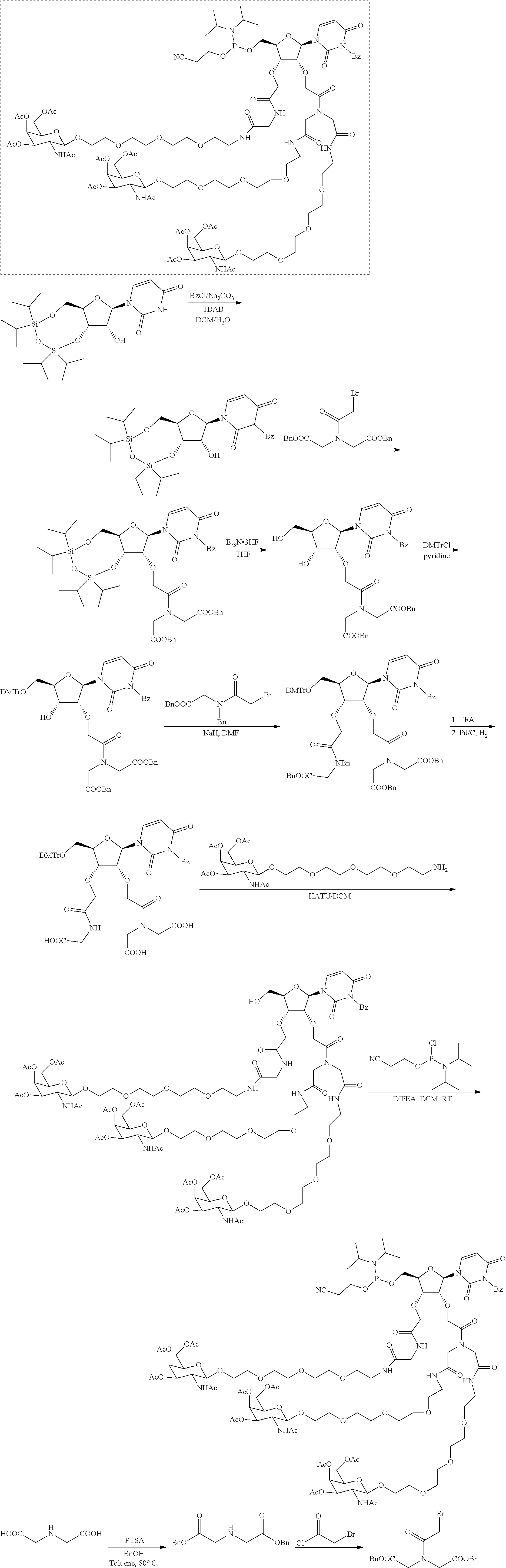

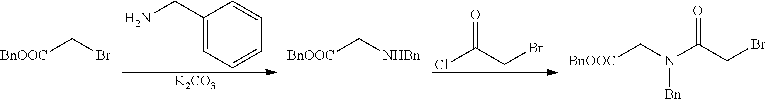

### Example 21

Exemplary compounds falling within the scope of the present disclosure could be synthesized according to the following scheme:

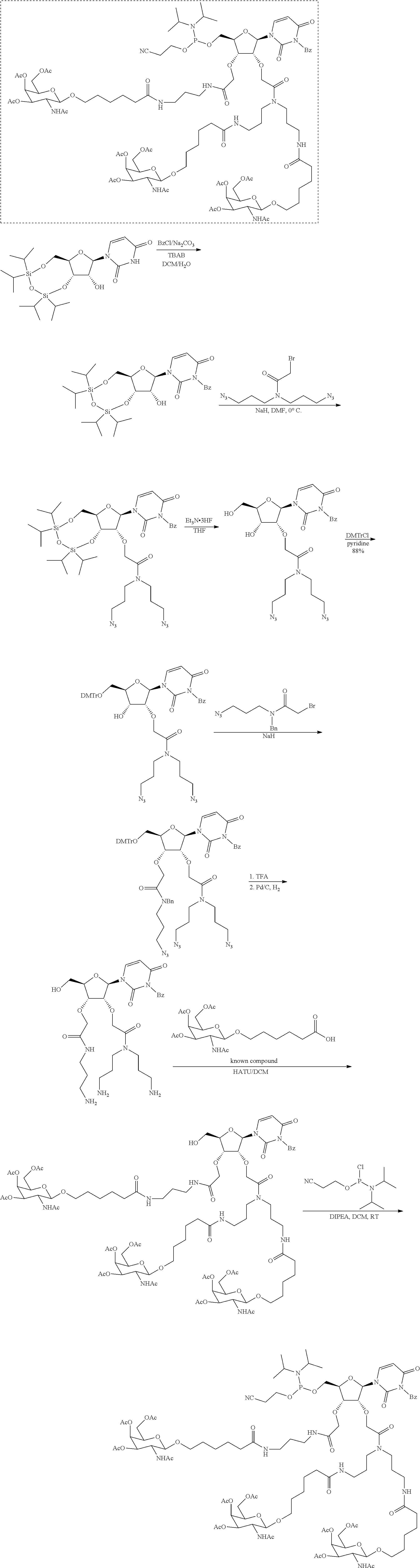

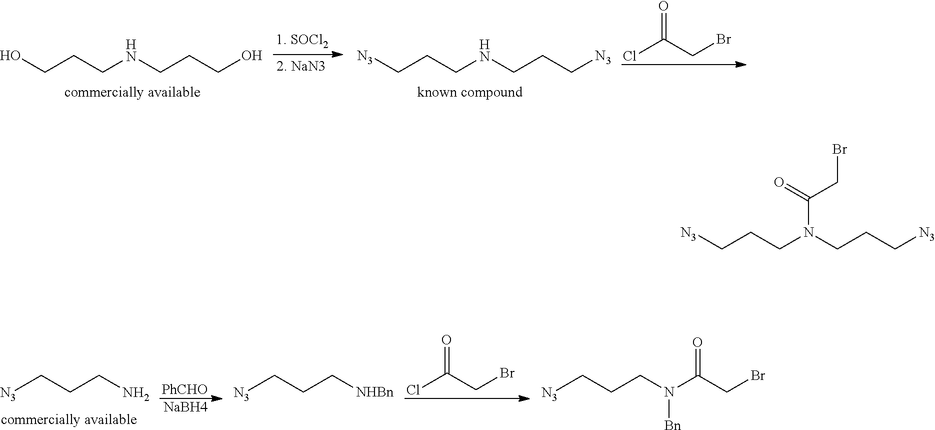

### Example 22

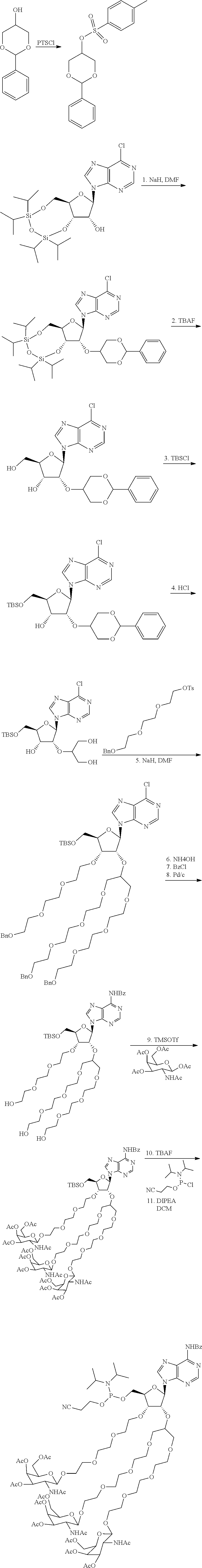

### Example 23

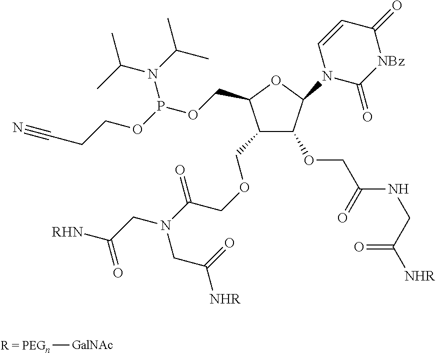

Exemplary compounds falling within the scope of the present disclosure could be synthesized according to the following scheme:

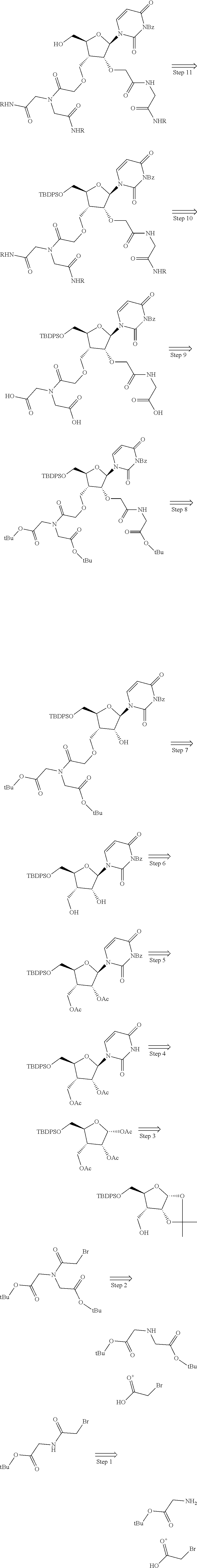

### INCORPORATION BY REFERENCE

The contents of all references (including literature references, issued patents, published patent applications, and co-pending patent applications) cited throughout this application are hereby expressly incorporated herein in their entireties by reference.

### Equivalents

Those skilled in the art will recognize, or be able to ascertain using no more than routine experimentation, many equivalents of the specific embodiments of the invention described herein. Such equivalents are intended with be encompassed by the following claims.

### Embodiments

Additional embodiments of the invention include:
1. A compound of Formula (I-a), (I-b), (I-c), or (I-d), or a salt, solvate, or hydrate thereof:

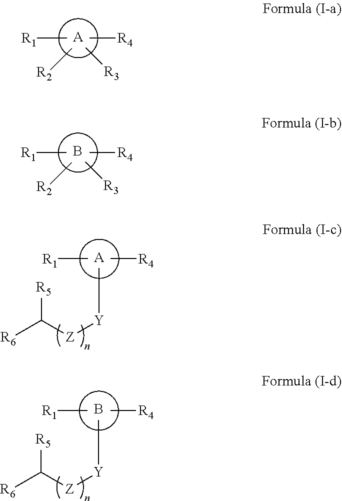

wherein:
[symbol] is Ring A, wherein each Ring A is independently optionally substituted carbocyclyl or optionally substituted heterocyclyl;
[symbol] is Ring B, wherein each Ring B is independently optionally substituted aryl or optionally substituted heteroaryl;
each n is independently 0, 1, 2, 3, or 4;
each Y is independently O, CH₂, S, S(═O), S(═O)₂, NH, substituted amino, NHC(═O), C(═O)NH, P(═O)₂—O—, P(═O)(═S)—O, P(═S)₂—O, —O—P(═O)₂—O—, —O—P(═O)(═S)—O—, —O—P(═S)₂—O—, —O—P(O)₂—, —O—P(═O)(═S)—, —O—P(═S)₂—;
each Z is independently an optionally substituted alkyl group, an optionally substituted alkenyl group, an optionally substituted alkynyl group, or ethylene glycol;
each R₁ is independently alkyl-O-phosphoramidite, alkyl phosphoramidite, optionally substituted alkenyl phosphoramidite, or optionally substituted alkynyl phosphoramidite;
R₂ and R₅ are each independently optionally substituted alkyl-O-GalNAc, polyethylene glycol-O-GalNAc, optionally substituted alkenyl-O-GalNAc, optionally substituted alkynyl-O-GalNAc, alkyl-S(═O)-alkyl-GalNAc, alkyl-S(═O)₂-alkyl-GalNAc, alkyl-S(═O)—NH-alkyl, alkyl-S(═O)₂—NH-alkyl-GalNAc, alkyl-P P(═O)(—O—)—NH-alkyl-GalNAc, alkyl-O—P(═O)(—O—)—O-alkyl-GalNAc, alkyl-O—P(—O—)(═S)—O-alkyl-GalNAc, or alkyl-O—P(—S—)(═S)—O-alkyl-GalNAc;
R₃ and R₆ are each independently optionally substituted alkyl-O-GalNAc, optionally substituted alkenyl-O-GalNAc, optionally substituted alkynyl-O-GalNAc, alkyl-S(═O)-alkyl-GalNAc, alkyl-S(═O)₂-alkyl-GalNAc, alkyl-S(═O)—NH-alkyl, alkyl-S(═O)₂—NH-alkyl-GalNAc, alkyl-P P(═O)(—O—)—NH-alkyl-GalNAc, alkyl-O—P(═O)(—O—)—O-alkyl-GalNAc, alkyl-O—P(—O—)(═S)—O-allyl-GalNAc, or alkyl-O—P(—S—)(═S)—O-alkyl-GalNAc; and
each R₄ is independently optionally substituted alkyl-O-GalNAc, optionally substituted alkenyl-O-GalNAc, optionally substituted alkynyl-O-GalNAc, alkyl-S(═O)-alkyl-GalNAc, alkyl-S(═O)₂-alkyl-GalNAc, alkyl-S(═O)—NH-alkyl, alkyl-S(═O)₂—NH-alkyl-GalNAc, alkyl-P P(═O)(—O—)—NH-alkyl-GalNAc, alkyl-O—P(═O)(—O—)—O-alkyl-GalNAc, alkyl-O—P(—O—)(═S)—O-alkyl-GalNAc, or alkyl-O—P(—S—)(═S)—O-alkyl-GalNAc.
2. The compound of embodiment 1, or a salt, solvate, or hydrate thereof, having Formula (I-a).
3. The compound of embodiment 1 or 2, wherein R₂ and R₃ are the same.
4. The compound of embodiment 1, or a salt, solvate, or hydrate thereof, having Formula (I-c).
5. The compound of any of embodiments 1-4, or a salt, solvate, or hydrate thereof, wherein Ring A is optionally substituted carbocyclyl.
6. The compound of any of embodiments 1-4, or a salt, solvate, or hydrate thereof, wherein Ring A is optionally substituted heterocyclyl.
7. The compound of embodiment 1, or a salt, solvate, or hydrate thereof, having Formula (I-b).
8. The compound of embodiment 1 or 7, wherein R₂ and R₃ are the same.
9. The compound of embodiment 1, or a salt, solvate, or hydrate thereof, having Formula (I-d).
10. The compound of any of embodiments 1 or 7-9, or a salt, solvate, or hydrate thereof, wherein Ring B is optionally substituted aryl.
11. The compound of any of embodiments 1, or 7-9, or a salt, solvate, or hydrate thereof, wherein Ring B is optionally substituted heteroaryl.
12. A compound of Formula (II-a), (II-b), or (II-c), or a salt, solvate, or hydrate thereof:

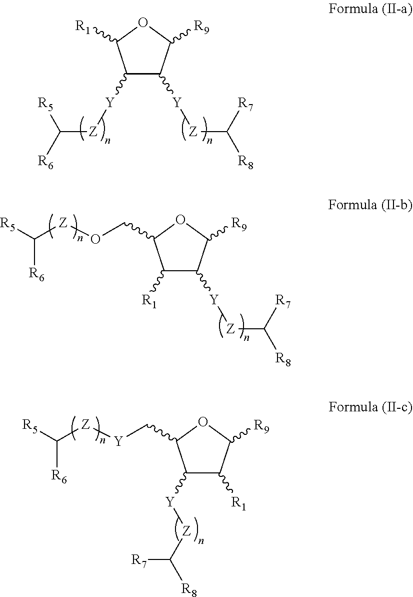

wherein:
each n is independently 0, 1, 2, 3, or 4;
each Y is independently O, CH₂, S, S(═O), S(═O)₂, NH, substituted N group, NHC(═O), C(═O)NH, P(═O)₂—O—, P(═O)(═S)—O, P(═S)₂—O, —O—P(═O)₂—O—, —O—P(═O)(═S)—O—, —O—P(═S)₂—O—, —O—P(═O)₂—, —O—P(═O)(═S)—, —O—P(═S)₂—;
each Z is independently an optionally substituted alkyl group, an optionally substituted alkenyl group, an optionally substituted alkynyl group, or ethylene glycol;
each R₁ is independently alkyl-O-phosphoramidite, optionally substituted alkenyl phosphoramidite, or optionally substituted alkynyl phosphoramidite;
R₅, R₆, R₇, and R₈ are each independently optionally substituted alkyl-O-GalNAc, polyethylene glycol-O-GalNAc, optionally substituted alkenyl-O-GalNAc, optionally substituted alkynyl-O-GalNAc, alkyl-S(═O)-alkyl-GalNAc, alkyl-S(═O)₂-alkyl-GalNAc, alkyl-S(═O)—NH-alkyl, alkyl-S(═O)₂—NH-alkyl-GalNAc, alkyl-P P(═O)(—O—)—NH-alkyl-GalNAc, alkyl-O—P(═O)(—O—)—O-alkyl-GalNAc, alkyl-O—P(—O—)(═S)—O-alkyl-GalNAc, or alkyl-O—P(—S—)(═S)—O-alkyl-GalNAc; and
R₉ is H, adenine, guanine, thymine, cytosine, uracil, inosine (I), or a nucleobase variant.
13. The compound of embodiment 12, or a salt, solvate, or hydrate thereof, having Formula (II-a).
14. The compound of embodiment 12, or a salt, solvate, or hydrate thereof, having Formula (II-b).
15. The compound of embodiment 12, or a salt, solvate, or hydrate thereof, having Formula (II-c).
16. The compound of any of embodiments 12-15, or a salt, solvate, or hydrate thereof, wherein three of R₅, R₆, R₇, and R₈ are each independently optionally substituted alkyl-O-GalNAc, polyethylene glycol-O-GalNAc, optionally substituted alkenyl-O-GalNAc, optionally substituted alkynyl-O-GalNAc, alkyl-S(═O)-alkyl-GalNAc, alkyl-S(═O)₂-alkyl-GalNAc, alkyl-S(═O)—NH-alkyl, alkyl-S(═O)₂—NH-alkyl-GalNAc, alkyl-P P(═O)(—O—)—NH-alkyl-GalNAc, alkyl-O—P(═O)(—O—)—O-alkyl-GalNAc, alkyl-O—P(—O—)(═S)—O-alkyl-GalNAc, or alkyl-O—P(—S—)(═S)—O-alkyl-GalNAc, and the remaining one of R₅, R₆, R₇, or R₈ is independently H, alkyl, alkenyl, alkynyl, halogen, substituted amine, thiol, or amide.
17. The compound of any of embodiments 12-16, or a salt, solvate, or hydrate thereof, wherein two of R₅, R₆, R₇, and R₈ are each the same.
18. The compound of any of embodiments 12-16, or a salt, solvate, or hydrate thereof, wherein three of R₅, R₆, R₇, and R₈ are each the same.
19. The compound of any of embodiments 12-16, or a salt, solvate, or hydrate thereof, wherein three of R₅, R₆, R₇, and R₈ are each the same, and the remaining one of R₅, R₆, R₇, or R₈ is independently H, alkyl, alkenyl, alkynyl, halogen, substituted amine, thiol, or amide.
20. The compound of any of embodiments 12-19, or a salt, solvate, or hydrate thereof, wherein R₉ is H.
21. The compound of any of embodiments 12-19, or a salt, solvate, or hydrate thereof, wherein R₉ is adenine, guanine, thymine, cytosine, or uracil.
22. A compound of Formula (III-a) or (III-b) or a salt, solvate, or hydrate thereof:

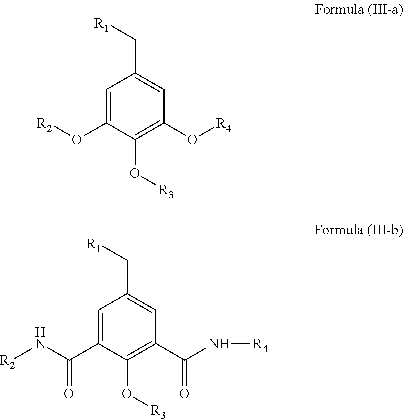

wherein:
each R₁ is independently alkyl-O-phosphoramidite, alkyl phosphoramidite, optionally substituted alkenyl phosphoramidite, or optionally substituted alkynyl phosphoramidite;
each R₂ is independently optionally substituted alkyl-O-GalNAc, polyethylene glycol-O-GalNAc, optionally substituted alkenyl-O-GalNAc, optionally substituted alkynyl-O-GalNAc, alkyl-S(═O)-alkyl-GalNAc, alkyl-S(═O)₂-alkyl-GalNAc, alkyl-S(═O)—NH-alkyl, alkyl-S(═O)₂—NH-alkyl-GalNAc, alkyl-P P(═O)(—O—)—NH-alkyl-GalNAc, alkyl-O—P(═O)(—O—)—O-alkyl-GalNAc, alkyl-O—P(—O—)(═S)—O-alkyl-GalNAc, or alkyl-O—P(—S—)(═S)—O-alkyl-GalNAc;
each R₃ is independently optionally substituted alkyl-O-GalNAc, polyethylene glycol-O-GalNAc, optionally substituted alkenyl-O-GalNAc, optionally substituted alkynyl-O-GalNAc, alkyl-S(═O)-alkyl-GalNAc, alkyl-S(═O)₂-alkyl-GalNAc, alkyl-S(═O)—NH-alkyl, alkyl-S(═O)₂—NH-alkyl-GalNAc, alkyl-P P(═O)(—O—)—NH-alkyl-GalNAc, alkyl-O—P(═O)(—O—)—O-alkyl-GalNAc, alkyl-O—P(—O—)(═S)—O-alkyl-GalNAc, or alkyl-O—P(—S—)(═S)—O-alkyl-GalNAc; and
each R₄ is independently optionally substituted alkyl-O-GalNAc, polyethylene glycol-O-GalNAc, optionally substituted alkenyl-O-GalNAc, optionally substituted alkynyl-O-GalNAc, alkyl-S(═O)-alkyl-GalNAc; alkyl-S(═O)₂-alkyl-GalNAc, alkyl-S(═O)—NH-alkyl, alkyl-S(═O)₂—NH-alkyl-GalNAc, alkyl-P P(═O)(—O—)—NH-alkyl-GalNAc, alkyl-O—P(═O)(—O—)—O-alkyl-GalNAc, alkyl-O—P(—O—)(═S)—O-alkyl-GalNAc, or alkyl-O—P(—S—)(═S)—O-alkyl-GalNAc.
23. The compound of embodiment 22, or a salt, solvate, or hydrate thereof, having Formula (III-a).
24. The compound of embodiment 22, or a salt, solvate, or hydrate thereof, having Formula (III-b).
25. A compound of the formula:

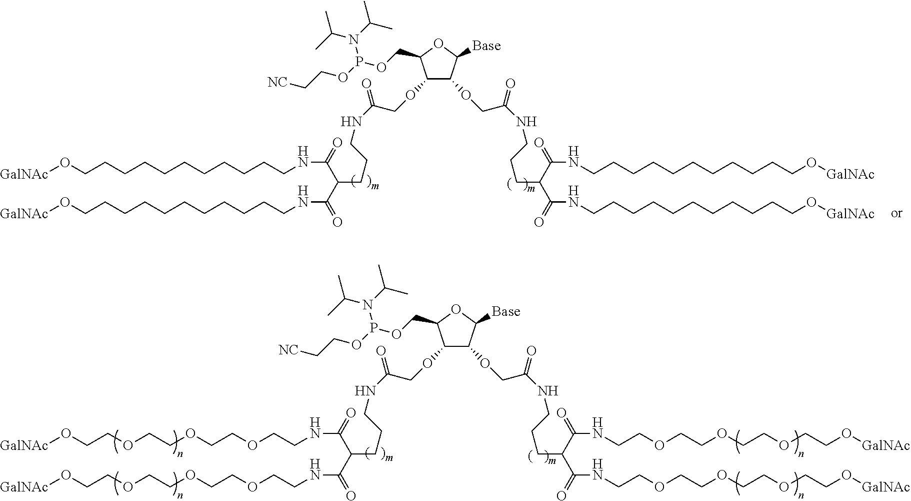

or a salt, solvate, or hydrate thereof,
wherein:
each n is independently an integer 0-10, inclusive;
each m is independently an integer 0-10, inclusive; and
base is adenosine, uracil, thymine, cytosine, inosine, or guanosine.
26. A compound of Formula (IV-a), (IV-b), (IV-c), or (IV-d):

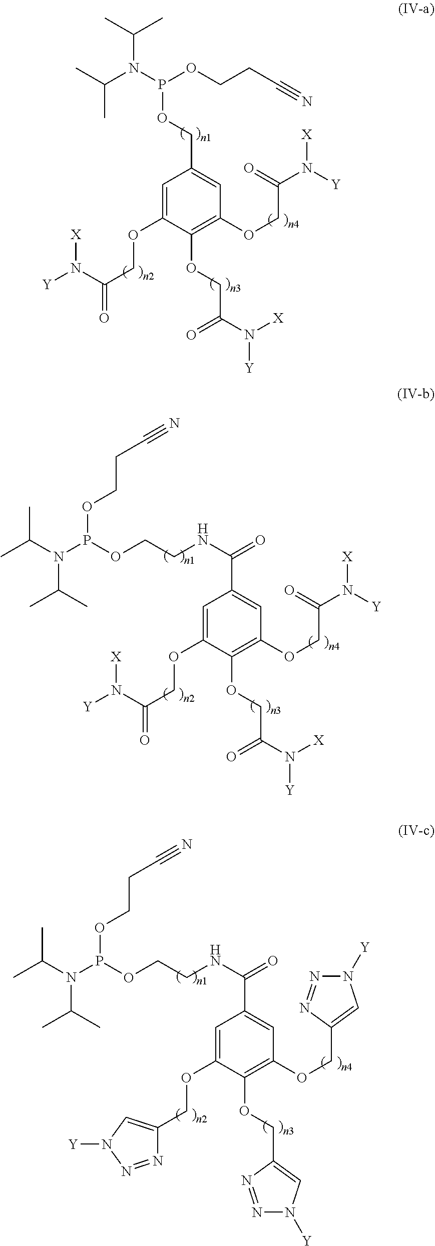

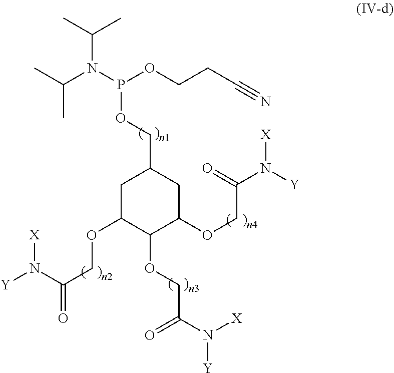

or a salt, solvate, or hydrate thereof,
wherein:
n1 is independently an integer 1-10, inclusive;
n2 is independently an integer 1-10, inclusive;
n3 is independently an integer 1-10, inclusive;
n4 is independently an integer 1-10, inclusive;
each X is independently H, alkyl-GalNAc, or PEG-GalNAc; and
each Y is independently H, alkyl-GalNAc, or PEG-GalNAc.
27. A modified oligonucleotide comprising the compound of any one of embodiments 1-26.
28. The modified oligonucleotide of embodiment 27, wherein the oligonucleotide comprises an siRNA, an miRNA, an ADAR recruiting molecule, an ADAR targeting molecule, a guide RNA, or an antisense nucleic acid.
29. A compound of Formula (V), or salt, solvate, or hydrate thereof:

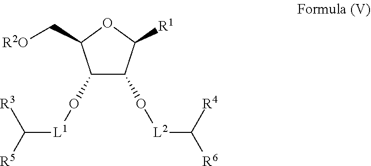

wherein:
R¹ is H, adenine, guanine, thymine, cytosine, or uracil;
R² is H, Protecting Group (PG),

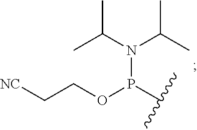 [SMILES: *P(OCCC#N)N(C(C)C)C(C)C]

L¹ is alkyl, or alkyl-C(═O)—NH-alkyl;
L² is alkyl, or alkyl-C(═O)—NH-alkyl;
R³ is H, —C═(O)—NH—(CH₂CH₂O)ⱼ-GalNAc, or —C(═O)—NH-alkyl-NH—C(═O)-alkyl-O-GalNAc;
R⁴ is H, —C═(O)—NH—(CH₂CH₂O)ₖ-GalNAc, or —C(═O)—NH-alkyl-NH—C(═O)-alkyl-O-GalNAc;
R⁵ is —C═(O)—NH—(CH₂CH₂O)ₘ-GalNAc, or —C(═O)—NH-alkyl-NH—C(═O)-alkyl-O-GalNAc;
R⁶ is —C═(O)—NH—(CH₂CH₂O)ₙ-GalNAc, or —C(═O)—NH-alkyl-NH—C(═O)-alkyl-O-GalNAc;
j is an integer 1-10, inclusive;
k is an integer 1-10, inclusive;
m is an integer 1-10, inclusive; and
n is an integer 1-10, inclusive.
30. The compound of embodiment 29, or a salt, solvate, or hydrate thereof, wherein R³, R⁴, R⁵, and R⁶ are the same.
31. The compound of embodiment 29 or 30, or a salt, solvate, or hydrate thereof, wherein R³, R⁵, and R⁶ are the same.
32. The compound of any one of embodiments 29-31, or a salt, solvate, or hydrate thereof, wherein R³ or R⁴ is H.
33. The compound of any one of embodiments 29-32, or a salt, solvate, or hydrate thereof, wherein L¹ and L² are the same.
34. The compound of any one of embodiments 29-33, or a salt, solvate, or hydrate thereof, wherein
L¹ and L² are each independently alkyl;
R³ is H, —C═(O)—NH—(CH₂CH₂O)ⱼ-GalNAc, or —C(═O)—NH-alkyl-NH—C(═O)-alkyl-O-GalNAc;
R⁴ is H, —C═(O)—NH—(CH₂CH₂O)ₖ-GalNAc, or —C(═O)—NH-alkyl-NH—C(═O)-alkyl-O-GalNAc;
R⁵ is —C═(O)—NH—(CH₂CH₂O)ₘ-GalNAc, or —C(═O)—NH-alkyl-NH—C(═O)-alkyl-O-GalNAc;
R⁶ is —C═(O)—NH—(CH₂CH₂O)ₙ-GalNAc, or —C(═O)—NH-alkyl-NH—C(═O)-alkyl-O-GalNAc.
35. The compound of any one of embodiments 29-34, or a salt, solvate, or hydrate thereof, wherein
L¹ and L² are each independently alkyl-C(═O)—NH-alkyl;
R³ is H, —C═(O)—NH—(CH₂CH₂O)ⱼ-GalNAc, or —C(═O)—NH-alkyl-NH—C(═O)-alkyl-O-GalNAc;
R⁴ is H, —C═(O)—NH—(CH₂CH₂O)ₖ-GalNAc, or —C(═O)—NH-alkyl-NH—C(═O)-alkyl-O-GalNAc;
R⁵ is —C═(O)—NH—(CH₂CH₂O)ₘ-GalNAc, or —C(═O)—NH-alkyl-NH—C(═O)-alkyl-O-GalNAc;
R⁶ is —C═(O)—NH—(CH₂CH₂O)ₙ-GalNAc, or —C(═O)—NH-alkyl-NH—C(═O)-alkyl-O-GalNAc.
36. The compound of any one of embodiments 29-35, or a salt, solvate, or hydrate thereof, wherein R⁴ is H.
37. The compound of any one of embodiments 29-36, or a salt, solvate, or hydrate thereof, wherein
L¹ and L² are each independently alkyl;
R³ is —C═(O)—NH—(CH₂CH₂O)ⱼ-GalNAc, or —C(═O)—NH-alkyl-NH—C(═O)-alkyl-O-GalNAc;
R⁴ is H;
R⁵ is —C═(O)—NH—(CH₂CH₂O)ₘ-GalNAc, or —C(═O)—NH-alkyl-NH—C(═O)-alkyl-O-GalNAc;
R⁶ is —C═(O)—NH—(CH₂CH₂O)ₙ-GalNAc, or —C(═O)—NH-alkyl-NH—C(═O)-alkyl-O-GalNAc.
38. The compound of any one of embodiments 29-37, or a salt, solvate, or hydrate thereof, wherein
L¹ and L² are each independently alkyl-C(═O)—NH-alkyl;
R³ is —C═(O)—NH—(CH₂CH₂O)ⱼ-GalNAc, or —C(═O)—NH-alkyl-NH—C(═O)-alkyl-O-GalNAc;
R⁴ is H;
R⁵ is —C═(O)—NH—(CH₂CH₂O)ₘ-GalNAc, or —C(═O)—NH-alkyl-NH—C(═O)-alkyl-O-GalNAc;
R⁶ is —C═(O)—NH—(CH₂CH₂O)ₙ-GalNAc, or —C(═O)—NH-alkyl-NH—C(═O)-alkyl-O-GalNAc.
39. The compound of any one of embodiments 29-38, or a salt, solvate, or hydrate thereof, wherein
R³ is —C═(O)—NH—(CH₂CH₂O)ⱼ-GalNAc;
R⁴ is H;
R⁵ is —C═(O)NH—(CH₂CH₂O)ₘ-GalNAc;
R⁶ is —C═(O)—NH—(CH₂CH₂O)ₙ-GalNAc.
40. The compound of any one of embodiments 29-39, or a salt, solvate, or hydrate thereof, wherein
R³ is —C(═O)—NH-alkyl-NH—C(═O)-alkyl-O-GalNAc;
R⁴ is H;
R⁵ is —C(═O)—NH-alkyl-NH—C(═O)-alkyl-O-GalNAc;
R⁶ is —C(═O)—NH-alkyl-NH—C(═O)-alkyl-O-GalNAc.
41. The compound of any one of embodiments 29-40, or salt, solvate, or hydrate thereof, wherein j, m, and n are each independently an integer 4-10, inclusive.
42. The compound of any one of embodiments 29-41, or salt, solvate, or hydrate thereof, wherein j, m, and n are each the same integer 4-10, inclusive.
43. The compound of any one of embodiments 29-42, or salt, solvate, or hydrate thereof, wherein R¹ is H.
44. The compound of any one of embodiments 29-43, or salt, solvate, or hydrate thereof, wherein R¹ is adenine, guanine, thymine, cytosine, or uracil.
45. The compound of any one of embodiments 29-44, or salt, solvate, or hydrate thereof, wherein R¹ is adenine.
46. The compound of any one of embodiments 29-45, or salt, solvate, or hydrate thereof, wherein R¹ is guanine
47. The compound of any one of embodiments 29-46, or salt, solvate, or hydrate thereof, wherein R¹ is thymine.
48. The compound of any one of embodiments 29-47, or salt, solvate, or hydrate thereof, wherein R¹ is cytosine.
49. The compound of any one of embodiments 29-48, or salt, solvate, or hydrate thereof, wherein R¹ is uracil.
50. The compound of any one of embodiments 29-49, or salt, solvate, or hydrate thereof, wherein R² is H.
51. The compound of any one of embodiments 29-50, or salt, solvate, or hydrate thereof, wherein R² is Protecting Group (PG).
52. The compound of any one of embodiments 29-51, or salt, solvate, or hydrate thereof, wherein PG is an oxygen protecting group.
53. The compound of any one of embodiments 29-52, or salt, solvate, or hydrate thereof, wherein R² is

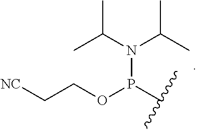 [SMILES: *P(OCCC#N)N(C(C)C)C(C)C]

54. The compound of any one of embodiments 29-53, or salt, solvate, or hydrate thereof, wherein for use as a reagent in a chemical reaction.
55. A compound of Formula (VI), or salt, solvate, or hydrate thereof:

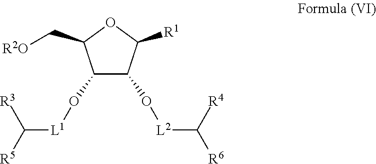

wherein:
R¹ is H, adenine, guanine, thymine, cytosine, or uracil;
R² is an oligonucleotide sequence;
L¹ is alkyl, or alkyl-C(═O)—NH-alkyl;
L² is alkyl, or alkyl-C(═O)—NH-alkyl;
R³ is H, —C═(O)—NH—(CH₂CH₂O)ⱼ-GalNAc, or —C(═O)—NH-alkyl-NH—C(═O)-alkyl-O-GalNAc;
R⁴ is H, —C═(O)—NH—(CH₂CH₂O)ₖ-GalNAc, or —C(═O)—NH-alkyl-NH—C(═O)-alkyl-O-GalNAc;
R⁵ is —C═(O)—NH—(CH₂CH₂O)ₘ-GalNAc, or —C(═O)—NH-alkyl-NH—C(═O)-alkyl-O-GalNAc;
R⁶ is —C═(O)—NH—(CH₂CH₂O)ₙ-GalNAc, or —C(═O)—NH-alkyl-NH—C(═O)-alkyl-O-GalNAc;
j is an integer 1-10, inclusive;
k is an integer 1-10, inclusive;
m is an integer 1-10, inclusive; and
n is an integer 1-10, inclusive.
56. A modified oligonucleotide comprising the compound of embodiment 55, wherein the oligonucleotide comprises an siRNA, an miRNA, an ADAR recruiting molecule, an ADAR targeting molecule, a guide RNA, or an antisense nucleic acid.
57. An oligonucleotide comprising a moiety of Formula (VII), or salt, solvate, or hydrate thereof:

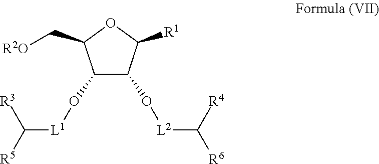

wherein:
R¹ is H, adenine, guanine, thymine, cytosine, or uracil;
R² is a bond;
L¹ is alkyl, or alkyl-C(═O)—NH-alkyl;
L² is alkyl, or alkyl-C(═O)—NH-alkyl;
R³ is H, —C═(O)—NH—(CH₂CH₂O)ⱼ-GalNAc, or —C(═O)—NH-alkyl-NH—C(═O)-alkyl-O-GalNAc;
R⁴ is H, —C═(O)—NH—(CH₂CH₂O)ₖ-GalNAc, or —C(═O)—NH-alkyl-NH—C(═O)-alkyl-O-GalNAc;
R⁵ is —C═(O)—NH—(CH₂CH₂O)ₘ-GalNAc, or —C(═O)—NH-alkyl-NH—C(═O)-alkyl-O-GalNAc;
R⁶ is —C═(O)—NH—(CH₂CH₂O)ₙ-GalNAc, or —C(═O)—NH-alkyl-NH—C(═O)-alkyl-O-GalNAc;
j is an integer 1-10, inclusive;
k is an integer 1-10, inclusive;
m is an integer 1-10, inclusive; and
n is an integer 1-10, inclusive.
58. The compound of embodiment 29, where the oligonucleotide comprises an siRNA, an miRNA, an ADAR recruiting molecule, an ADAR targeting molecule, a guide RNA, or an antisense nucleic acid.
59. A composition comprising a compound of any of embodiments 1-58, and a pharmaceutically acceptable carrier.
60. A method for modulating protein function in a subject, comprising administration of a compound of any of embodiments 1-58 to the subject.
61. A method for treating or ameliorating a disease, disorder, or symptom thereof in a subject, comprising administration of a compound of any of embodiments 1-58 to the subject.
62. A compound of Formula (I-a) or (I-c), or a salt, solvate, or hydrate thereof:

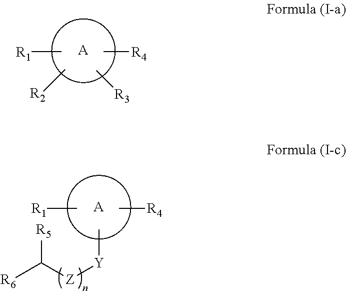

wherein:
[symbol] is Ring A, wherein each Ring A is independently optionally substituted carbocyclyl; each n is independently 0, 1, 2, 3, or 4;
each Y is independently O, CH₂, S, S(═O), S(═O)₂, NH, substituted amino, NHC(═O), C(═O)NH, P(═O)₂—O—, P(═O)(═S)—O, P(═S)₂—O, —O—P(═O)₂—O—, —O—P(═O)(═S)—O—, —O—P(═S)₂—O—, —O—P(O)₂—, —O—P(═O)(═S)—, —O—P(═S)₂—;
each Z is independently an optionally substituted alkyl group, an optionally substituted alkenyl group, an optionally substituted alkynyl group, or ethylene glycol;
each R₁ is independently alkyl-O-phosphoramidite, alkyl phosphoramidite, optionally substituted alkenyl phosphoramidite, or optionally substituted alkynyl phosphoramidite;
R₂ and R₅ are each independently optionally substituted alkyl-O-GalNAc, polyethylene glycol-O-GalNAc, optionally substituted alkenyl-O-GalNAc, optionally substituted alkynyl-O-GalNAc, alkyl-S(═O)-alkyl-GalNAc, alkyl-S(═O)₂-alkyl-GalNAc, alkyl-S(═O)—NH-alkyl, alkyl-S(═O)₂—NH-alkyl-GalNAc, alkyl-P P(═O)(—O—)—NH-alkyl-GalNAc, alkyl-O—P(═O)(—O—)—O-alkyl-GalNAc, alkyl-O—P(—O—)(═S)—O-alkyl-GalNAc, or alkyl-O—P(—S—)(═S)—O-alkyl-GalNAc;
R₃ and R₆ are each independently optionally substituted alkyl-O-GalNAc, optionally substituted alkenyl-O-GalNAc, optionally substituted alkynyl-O-GalNAc, alkyl-S(═O)-alkyl-GalNAc, alkyl-S(═O)₂-alkyl-GalNAc, alkyl-S(═O)—NH-alkyl, alkyl-S(═O)₂—NH-alkyl-GalNAc, alkyl-P P(═O)(—O—)—NH-alkyl-GalNAc, alkyl-O—P(═O)(—O—)—O-alkyl-GalNAc, alkyl-O—P(—O—)(═S)—O-allyl-GalNAc, or alkyl-O—P(—S—)(═S)—O-alkyl-GalNAc; and
each R₄ is independently optionally substituted alkyl-O-GalNAc, optionally substituted alkenyl-O-GalNAc, optionally substituted alkynyl-O-GalNAc, alkyl-S(═O)-alkyl-GalNAc, alkyl-S(═O)₂-alkyl-GalNAc, alkyl-S(═O)—NH-alkyl, alkyl-S(═O)₂—NH-alkyl-GalNAc, alkyl-P P(═O)(—O—)—NH-alkyl-GalNAc, alkyl-O—P(═O)(—O—)—O-alkyl-GalNAc, alkyl-O—P(—O—)(═S)—O-alkyl-GalNAc, or alkyl-O—P(—S—)(═S)—O-alkyl-GalNAc.
63. The compound of embodiment 62, wherein Ring A is not cyclopentyl.
64. A compound of Formula (I-a) or (I-c), or a salt, solvate, or hydrate thereof:

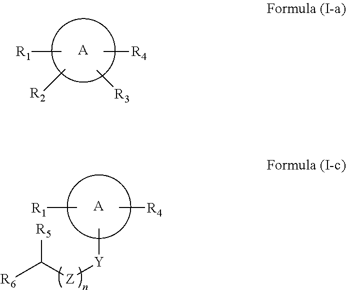

wherein:
[symbol] is Ring A, wherein each Ring A is independently optionally substituted heterocyclyl;
each n is independently 0, 1, 2, 3, or 4;
each Y is independently O, CH₂, S, S(═O), S(═O)₂, NH, substituted amino, NHC(═O),
C(═O)NH, P(═O)₂—O—, P(═O)(═S)—O, P(═S)₂—O, —O—P(═O)₂—O—, —O—P(═O)(═S)—O—, —O—P(═S)₂—O—, —O—P(═O)₂—, —O—P(═O)(═S)—, —O—P(═S)₂—;
each Z is independently an optionally substituted alkyl group, an optionally substituted alkenyl group, an optionally substituted alkynyl group, or ethylene glycol;
each R₁ is independently alkyl-O-phosphoramidite, alkyl phosphoramidite, optionally substituted alkenyl phosphoramidite, or optionally substituted alkynyl phosphoramidite;
R₂ and R₅ are each independently optionally substituted alkyl-O-GalNAc, polyethylene glycol-O-GalNAc, optionally substituted alkenyl-O-GalNAc, optionally substituted alkynyl-O-GalNAc, alkyl-S(═O)-alkyl-GalNAc, alkyl-S(═O)₂-alkyl-GalNAc, alkyl-S(═O)—NH-alkyl, alkyl-S(═O)₂—NH-alkyl-GalNAc, alkyl-P P(═O)(—O—)—NH-alkyl-GalNAc, alkyl-O—P(═O)(—O—)—O-alkyl-GalNAc, alkyl-O—P(—O—)(═S)—O-alkyl-GalNAc, or alkyl-O—P(—S—)(═S)—O-alkyl-GalNAc;
R₃ and R₆ are each independently optionally substituted alkyl-O-GalNAc, optionally substituted alkenyl-O-GalNAc, optionally substituted alkynyl-O-GalNAc, alkyl-S(═O)-alkyl-GalNAc, alkyl-S(═O)₂-alkyl-GalNAc, alkyl-S(═O)—NH-alkyl, alkyl-S(═O)₂—NH-alkyl-GalNAc, alkyl-P P(═O)(—O—)—NH-alkyl-GalNAc, alkyl-O—P(═O)(—O—)—O-alkyl-GalNAc, alkyl-O—P(—O—)(═S)—O-alkyl-GalNAc, or alkyl-O—P(—S—)(═S)—O-alkyl-GalNAc; and
each R₄ is independently optionally substituted alkyl-O-GalNAc, optionally substituted alkenyl-O-GalNAc, optionally substituted alkynyl-O-GalNAc, alkyl-S(═O)-alkyl-GalNAc, alkyl-S(═O)₂-alkyl-GalNAc, alkyl-S(═O)—NH-alkyl, alkyl-S(═O)₂—NH-alkyl-GalNAc, alkyl-P P(═O)(—O—)—NH-alkyl-GalNAc, alkyl-O—P(═O)(—O—)—O-alkyl-GalNAc, alkyl-O—P(—O—)(═S)—O-alkyl-GalNAc, or alkyl-O—P(—S—)(═S)—O-alkyl-GalNAc.
65. The compound of embodiment 64, wherein Ring A (i) contains only one heteroatom, which is oxygen, (ii) comprises at least one heteroatom that is S, or (iii) contains no nitrogen heteroatoms.
66. The compound of embodiment 64 or 65, wherein Ring A is oxetanyl, tetrahydrofuranyl, or tetrahydropyranyl.
67. The compound of any one of embodiment 64-66, wherein Ring A is tetrahydrofuranyl.
68. The compound of embodiment 64, wherein Ring A is not pyrrolidinyl, piperidinyl, or morpholinyl
69. A compound of Formula (I-b) or (I-d), or a salt, solvate, or hydrate thereof:

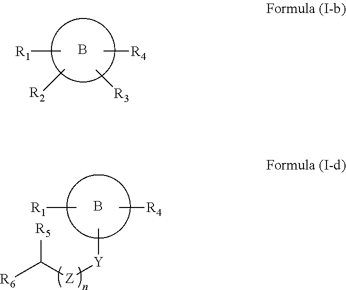

wherein:
[symbol] is Ring B, wherein each Ring B is independently optionally substituted aryl;
each n is independently 0, 1, 2, 3, or 4;
each Y is independently O, CH₂, S, S(═O), S(═O)₂, NH, substituted amino, NHC(═O), C(═O)NH, P(═O)₂—O—, P(═O)(═S)—O, P(═S)₂—O, —O—P(═O)₂—O—, —O—P(═O)(═S)—O—, —O—P(═S)₂—O—, —O—P(═O)₂—, —O—P(═O)(═S)—, —O—P(═S)₂—;
each Z is independently an optionally substituted alkyl group, an optionally substituted alkenyl group, an optionally substituted alkynyl group, or ethylene glycol;
each R₁ is independently alkyl-O-phosphoramidite, alkyl phosphoramidite, optionally substituted alkenyl phosphoramidite, or optionally substituted alkynyl phosphoramidite;
R₂ and R₅ are each independently optionally substituted alkyl-O-GalNAc, polyethylene glycol-O-GalNAc, optionally substituted alkenyl-O-GalNAc, optionally substituted alkynyl-O-GalNAc, alkyl-S(═O)-alkyl-GalNAc, alkyl-S(═O)₂-alkyl-GalNAc, alkyl-S(═O)—NH-alkyl, alkyl-S(═O)₂—NH-alkyl-GalNAc, alkyl-P P(═O)(—O—)—NH-alkyl-GalNAc, alkyl-O—P(═O)(—O—)—O-alkyl-GalNAc, alkyl-O—P(—O—)(═S)—O-alkyl-GalNAc, or alkyl-O—P(—S—)(═S)—O-alkyl-GalNAc;
R₃ and R₆ are each independently optionally substituted alkyl-O-GalNAc, optionally substituted alkenyl-O-GalNAc, optionally substituted alkynyl-O-GalNAc, alkyl-S(═O)-alkyl-GalNAc, alkyl-S(═O)₂-alkyl-GalNAc, alkyl-S(═O)—NH-alkyl, alkyl-S(═O)₂—NH-alkyl-GalNAc, alkyl-P P(═O)(—O—)—NH-alkyl-GalNAc, alkyl-O—P(═O)(—O—)—O-alkyl-GalNAc, alkyl-O—P(—O—)(═S)—O-alkyl-GalNAc, or alkyl-O—P(—S—)(═S)—O-alkyl-GalNAc; and
each R₄ is independently optionally substituted alkyl-O-GalNAc, optionally substituted alkenyl-O-GalNAc, optionally substituted alkynyl-O-GalNAc, alkyl-S(═O)-alkyl-GalNAc, alkyl-S(═O)₂-alkyl-GalNAc, alkyl-S(═O)—NH-alkyl, alkyl-S(═O)₂—NH-alkyl-GalNAc, alkyl-P P(═O)(—O—)—NH-alkyl-GalNAc, alkyl-O—P(═O)(—O—)—O-alkyl-GalNAc, alkyl-O—P(—O—)(═S)—O-alkyl-GalNAc, or alkyl-O—P(—S—)(═S)—O-alkyl-GalNAc.
70. The compound of embodiment 69, wherein Ring B is not phenyl.
71. A compound of Formula (I-b) or (I-d), or a salt, solvate, or hydrate thereof:

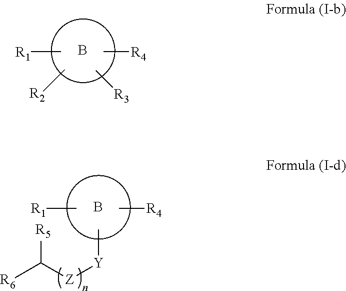

wherein:
[symbol] is Ring B, wherein each Ring B is independently optionally substituted heteroaryl;
each n is independently 0, 1, 2, 3, or 4;
each Y is independently O, CH₂, S, S(═O), S(═O)₂, NH, substituted amino, NHC(═O), C(═O)NH, P(═O)₂—O—, P(═O)(═S)—O, P(═S)₂—O, —O—P(═O)₂—O—, —O—P(═O)(═S)—O—, —O—P(═S)₂—O—, —O—P(═O)₂—, —O—P(═O)(═S)—, —O—P(═S)₂—;
each Z is independently an optionally substituted alkyl group, an optionally substituted alkenyl group, an optionally substituted alkynyl group, or ethylene glycol;
each R₁ is independently alkyl-O-phosphoramidite, alkyl phosphoramidite, optionally substituted alkenyl phosphoramidite, or optionally substituted alkynyl phosphoramidite;
R₂ and R₅ are each independently optionally substituted alkyl-O-GalNAc, polyethylene glycol-O-GalNAc, optionally substituted alkenyl-O-GalNAc, optionally substituted alkynyl-O-GalNAc, alkyl-S(═O)-alkyl-GalNAc, alkyl-S(═O)₂-alkyl-GalNAc, alkyl-S(═O)—NH-alkyl, alkyl-S(═O)₂—NH-alkyl-GalNAc, alkyl-P P(═O)(—O—)—NH-alkyl-GalNAc, alkyl-O—P(═O)(—O—)—O-alkyl-GalNAc, alkyl-O—P(—O—)(═S)—O-alkyl-GalNAc, or alkyl-O—P(—S—)(═S)—O-alkyl-GalNAc;
R₃ and R₆ are each independently optionally substituted alkyl-O-GalNAc, optionally substituted alkenyl-O-GalNAc, optionally substituted alkynyl-O-GalNAc, alkyl-S(═O)-alkyl-GalNAc, alkyl-S(═O)₂-alkyl-GalNAc, alkyl-S(═O)—NH-alkyl, alkyl-S(═O)₂—NH-alkyl-GalNAc, alkyl-P P(═O)(—O—)—NH-alkyl-GalNAc, alkyl-O—P(═O)(—O—)—O-alkyl-GalNAc, alkyl-O—P(—O—)(═S)—O-alkyl-GalNAc, or alkyl-O—P(—S—)(═S)—O-alkyl-GalNAc; and
each R₄ is independently optionally substituted allyl-O-GalNAc, optionally substituted alkenyl-O-GalNAc, optionally substituted alkynyl-O-GalNAc, alkyl-S(═O)-alkyl-GalNAc, alkyl-S(═O)₂-alkyl-GalNAc, alkyl-S(═O)—NH-alkyl, allyl-S(═O)₂—NH-alkyl-GalNAc, alkyl-P P(═O)(—O—)—NH-alkyl-GalNAc, alkyl-O—P(═O)(—O—)—O-alkyl-GalNAc, alkyl-O—P(—O—)(═S)—O-alkyl-GalNAc, or alkyl-O—P(—S—)(═S)—O-alkyl-GalNAc.
72. The compound of embodiment 71, wherein Ring B is not pyridinyl, 1,3-pyrimidinyl, 1,4-pyrimidinyl, 1-quinolinyl, or 9H-purinyl.
73. A compound of Formula (IV-a) or (IV-d):

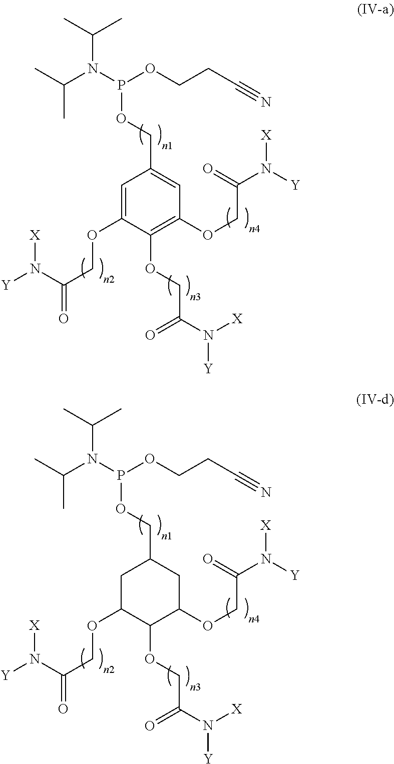

or a salt, solvate, or hydrate thereof,
wherein:
n1 is independently an integer 1-10, inclusive;
n2 is independently an integer 1-10, inclusive;
n3 is independently an integer 1-10, inclusive;
n4 is independently an integer 1-10, inclusive;
each X is independently H, alkyl-GalNAc, or PEG-GalNAc; and
each Y is independently H, alkyl-GalNAc, or PEG-GalNAc.
74. A modified oligonucleotide comprising the compound of any one of embodiments 1-26, wherein: (i) heterocyclyl contains only one heteroatom, which is oxygen, (ii) heterocyclyl comprises at least one heteroatom that is S; or (iii) heterocyclyl contains no nitrogen heteroatoms.
75. The modified oligonucleotide of embodiment 74, wherein the oligonucleotide comprises an siRNA, an miRNA, an ADAR recruiting molecule, an ADAR targeting molecule, a guide RNA, or an antisense nucleic acid.
76. The modified oligonucleotide of embodiment 27 or 28, wherein Ring A is not cyclopentyl, pyrrolidinyl, piperidinyl, or morpholinyl.
77. A modified oligonucleotide comprising the compound of any one of embodiments 1-26, wherein: (i) heteroaryl contains no nitrogen heteroatoms; (ii) heteroaryl contains only one heteroatom, which is oxygen; or (iii) heteroaryl contains only one heteroatom, which is sulfur.
78. The modified oligonucleotide of embodiment 77, wherein the oligonucleotide comprises an siRNA, an miRNA, an ADAR recruiting molecule, an ADAR targeting molecule, a guide RNA, or an antisense nucleic acid.
79. The modified oligonucleotide of embodiment 27 or 28, wherein Ring B is not phenyl, pyridinyl, 1,3-pyrimidinyl, 1,4-pyrimidinyl, 1-quinolinyl, or 9H-purinyl.
80. A compound of Formula (VIII), or salt, solvate, or hydrate thereof:

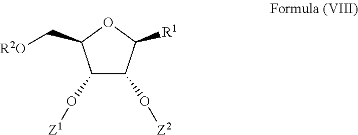

wherein:
R¹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, or optionally substituted heteroaryl;
R² is H, Protecting Group (PG),

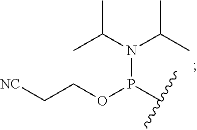 [SMILES: *P(OCCC#N)N(C(C)C)C(C)C]

Z¹ is

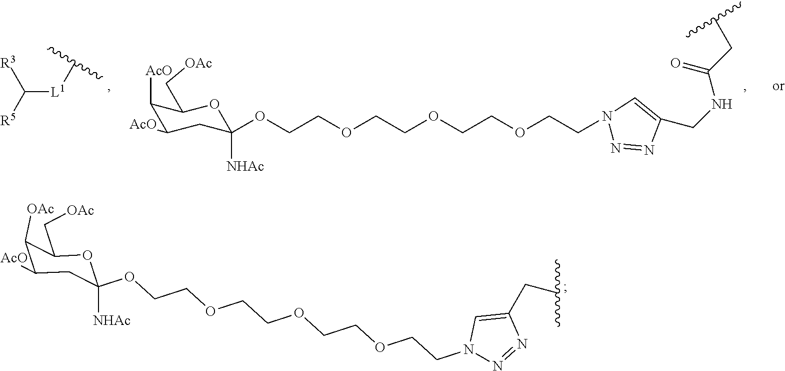

Z² is

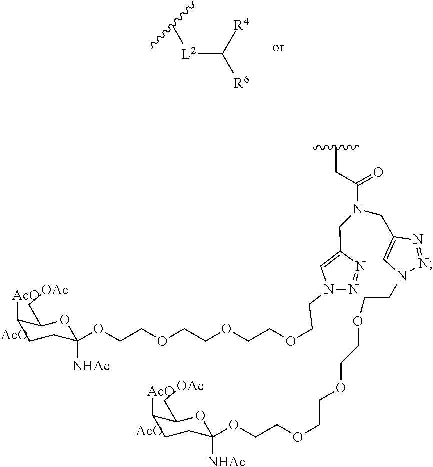

L¹ is alkyl, or alkyl-C(═O)—NH-alkyl;
L² is alkyl, or alkyl-C(═O)—NH-alkyl;
R³ is H, —C═(O)—NH—(CH₂CH₂O)ⱼ-GalNAc, or —C(═O)—NH-alkyl-NH—C(═O)-alkyl-O-GalNAc;
R⁴ is H, —C═(O)—NH—(CH₂CH₂O)ₖ-GalNAc, or —C(═O)—NH-alkyl-NH—C(═O)-alkyl-O-GalNAc;
R⁵ is —C═(O)—NH—(CH₂CH₂O)ₘ-GalNAc, or —C(═O)—NH-alkyl-NH—C(═O)-alkyl-O-GalNAc;
R⁶ is —C═(O)—NH—(CH₂CH₂O)ₙ-GalNAc, or —C(═O)—NH-alkyl-NH—C(═O)-alkyl-O-GalNAc;
j is an integer 1-10, inclusive;
k is an integer 1-10, inclusive;
m is an integer 1-10, inclusive; and
n is an integer 1-10, inclusive.
81. A compound of Formula (IX), or salt, solvate, or hydrate thereof:

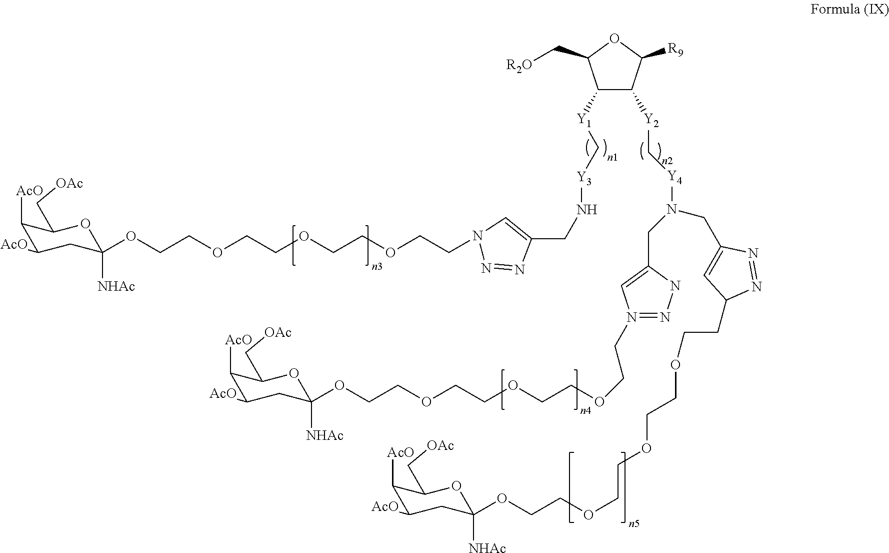

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, or optionally substituted heteroaryl;
R² is H, Protecting Group (PG), or

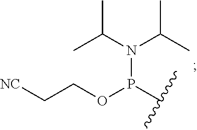 [SMILES: *P(OCCC#N)N(C(C)C)C(C)C]

Y₁ is O;
Y₂ is O;
Y₃ is CO, SO₂, P(O)O, CH₂—O—C(O), CH₂—NH—C(O), CH₂—NH—SO₂, or CH₂;
Y₄ is CO, SO₂, P(O)O, CH₂—O—C(O), CH₂—NH—C(O), CH₂—NH—SO₂, or CH₂;
n₂ is 1, 2, 3, 4, 5, or 6;
each n₁, n₃, n₄ and n₅ is independently 0, 1, 2, 3, 4, 5, or 6.
82. A compound of Formula (X), or salt, solvate, or hydrate thereof:

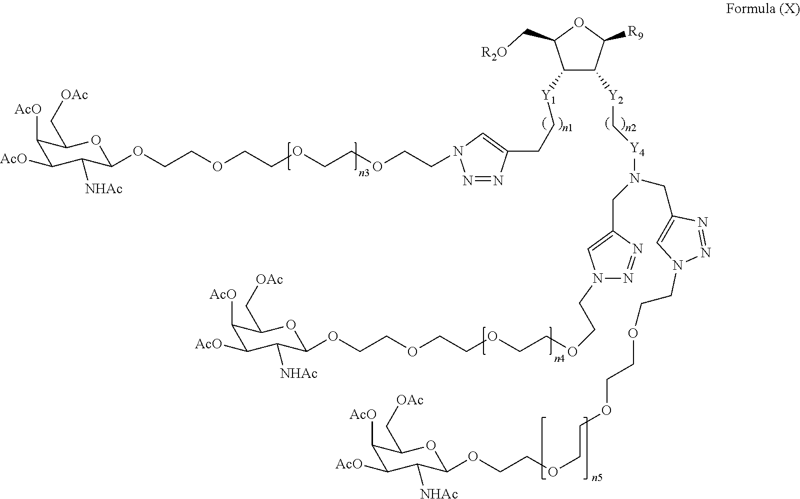

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, or optionally substituted heteroaryl;
R² is H, Protecting Group (PG), or

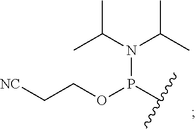 [SMILES: *P(OCCC#N)N(C(C)C)C(C)C]

Y₁ is O;
Y₂ is O;
Y₄ is CO, SO₂, P(O)O, CH₂—O—C(O), CH₂—NH—C(O), CH₂—NH—SO₂, or CH₂;
n₂ is 1, 2, 3, 4, 5, or 6;
each n₁, n₃, n₄ and n₅ is independently 0, 1, 2, 3, 4, 5, or 6.
83. The compound of embodiment 81 that is:

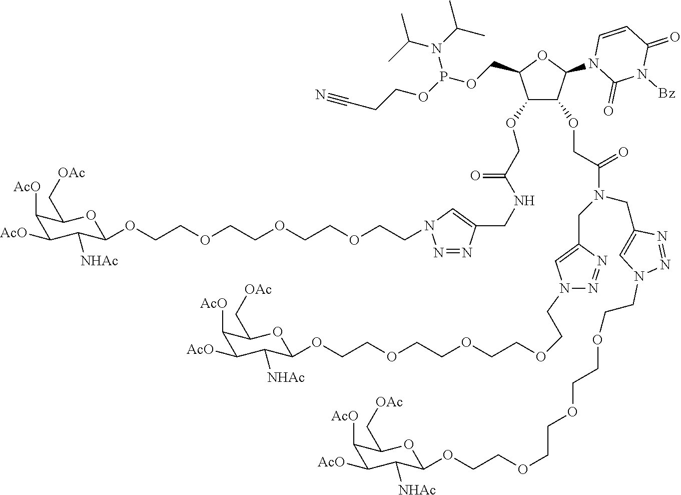

or salt, solvate, or hydrate thereof.
84. The compound of embodiment 82 that is:

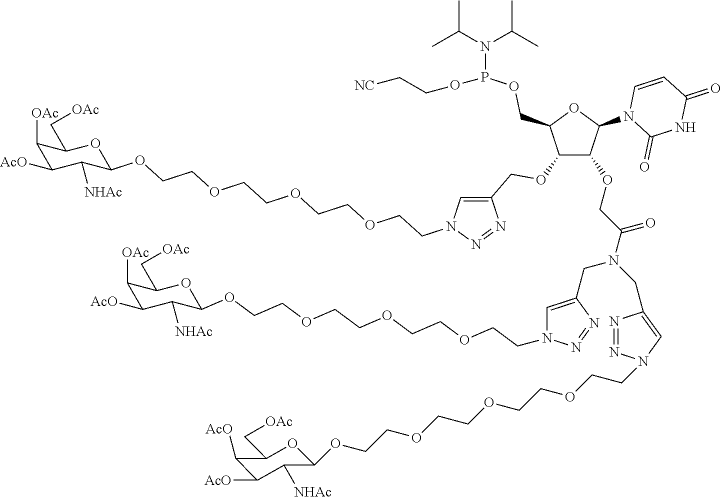 [SMILES: CC(=O)NC1C(OCCOCCOCCOCCn2cc(CO[C@H]3[C@@H](OCC(=O)N(Cc4cn(CCOCCOCCOCCOC5OC(COC(C)=O)C(OC(C)=O)C(OC(C)=O)C5NC(C)=O)nn4)Cc4cn(CCOCCOCCOCCOC5OC(COC(C)=O)C(OC(C)=O)C(OC(C)=O)C5NC(C)=O)nn4)[C@H](n4ccc(=O)[nH]c4=O)O[C@@H]3COP(OCCC#N)N(C(C)C)C(C)C)nn2)OC(COC(C)=O)C(OC(C)=O)C1OC(C)=O]

or salt, solvate, or hydrate thereof.
85. A compound of Formula (XI), or salt, solvate, or hydrate thereof:

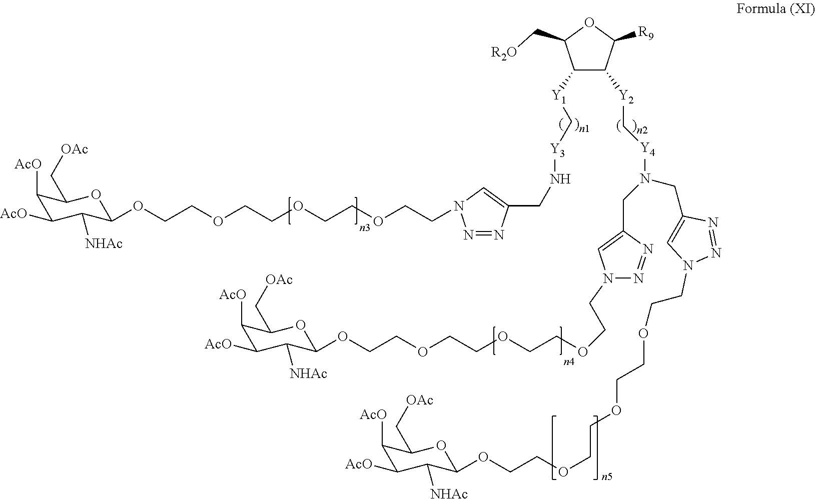

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, or optionally substituted heteroaryl;
R² is an oligonucleotide sequence;
Y₁ is O;
Y₂ is O;
Y₃ is CO, SO₂, P(O)O, CH₂—O—C(O), CH₂—NH—C(O), CH₂—NH—SO₂, or CH₂;
Y₄ is CO, SO₂, P(O)O, CH₂—O—C(O), CH₂—NH—C(O), CH₂—NH—SO₂, or CH₂;
n₂ is 1, 2, 3, 4, 5, or 6;
each n₁, n₃, n₄ and n₅ is independently 0, 1, 2, 3, 4, 5, or 6.
86. A compound of Formula (XII), or salt, solvate, or hydrate thereof:

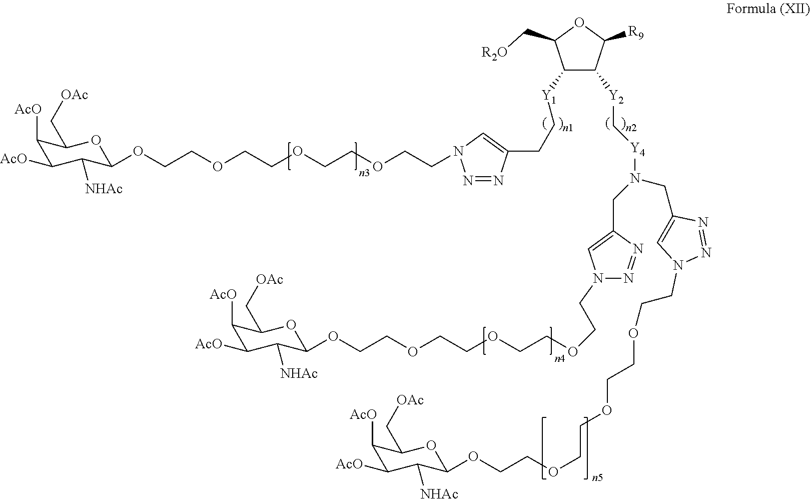

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, or optionally substituted heteroaryl;
R² is an oligonucleotide sequence;
Y₁ is O;
Y₂ is O;
Y₄ is CO, SO₂, P(O)O, CH₂—O—C(O), CH₂—NH—C(O), CH₂—NH—SO₂, or CH₂;
n₂ is 1, 2, 3, 4, 5, or 6;
each n₁, n₃, n₄ and n₅ is independently 0, 1, 2, 3, 4, 5, or 6.
87. An oligonucleotide comprising a moiety of Formula (XIII), or salt, solvate, or hydrate thereof:

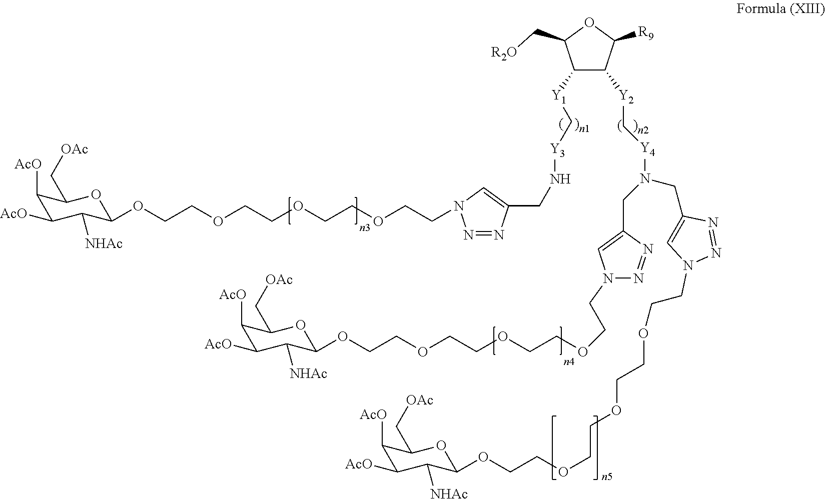

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, or optionally substituted heteroaryl;
R² is a bond;
Y₁ is O;
Y₂ is O;
Y₃ is CO, SO₂, P(O)O, CH₂—O—C(O), CH₂—NH—C(O), CH₂—NH—SO₂, or CH₂;
Y₄ is CO, SO₂, P(O)O, CH₂—O—C(O), CH₂—NH—C(O), CH₂—NH—SO₂, or CH₂;
n₂ is 1, 2, 3, 4, 5, or 6;
each n₁, n₃, n₄ and n₅ is independently 0, 1, 2, 3, 4, 5, or 6.
88. An oligonucleotide comprising a moiety of Formula (XIV), or salt, solvate, or hydrate thereof:

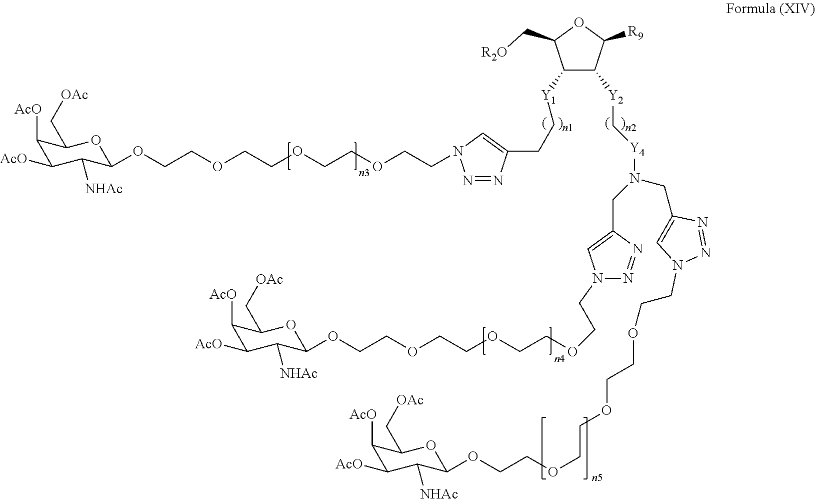

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, or optionally substituted heteroaryl;
R² is a bond;
Y₁ is O;
Y₂ is O;
Y₄ is CO, SO₂, P(O)O, CH₂—O—C(O), CH₂—NH—C(O), CH₂—NH—SO₂, or CH₂;
n₂ is 1, 2, 3, 4, 5, or 6;
each n₁, n₃, n₄ and n₅ is independently 0, 1, 2, 3, 4, 5, or 6.
89. A compound of Formula (XV), or a salt, solvate, or hydrate thereof:

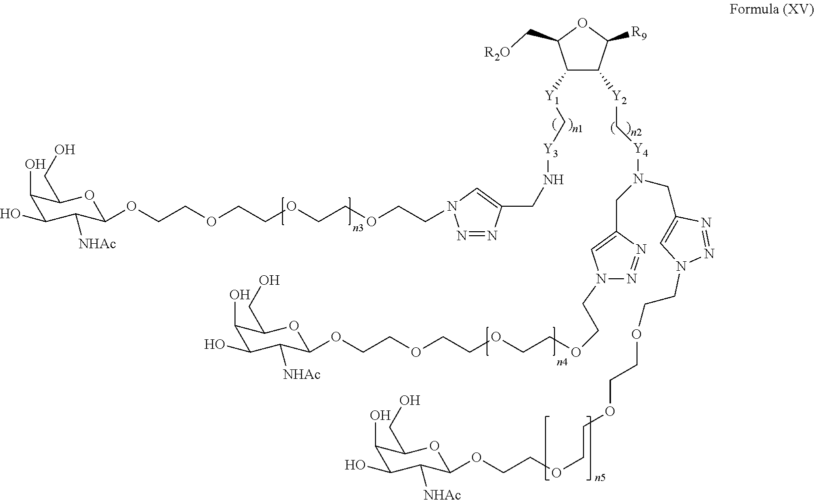

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, or optionally substituted heteroaryl;
R² is an oligonucleotide sequence;
Y₁ is O;
Y₂ is O;
Y₃ is CO, SO₂, P(O)O, CH₂—O—C(O), CH₂—NH—C(O), CH₂—NH—SO₂, or CH₂;
Y₄ is CO, SO₂, P(O)O, CH₂—O—C(O), CH₂—NH—C(O), CH₂—NH—SO₂, or CH₂;
n₂ is 1, 2, 3, 4, 5, or 6;
each n₁, n₃, n₄ and n₅ is independently 0, 1, 2, 3, 4, 5, or 6.
90. A compound of Formula (XVI), or a salt, solvate, or hydrate thereof:

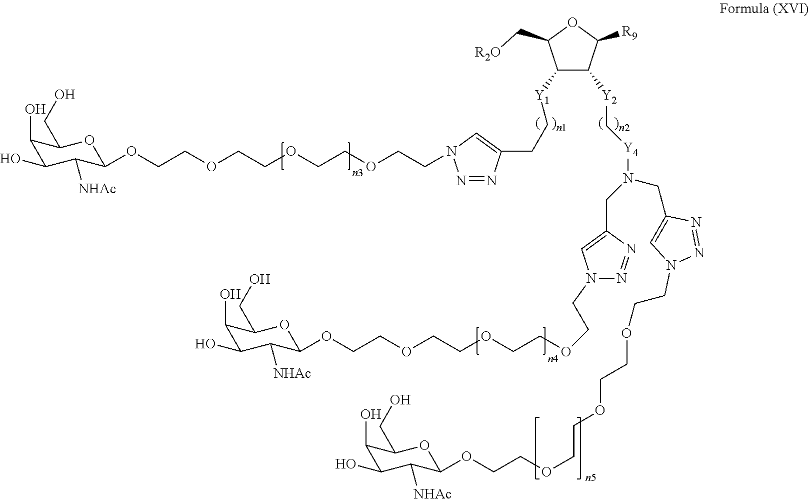

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, or optionally substituted heteroaryl;
R² is an oligonucleotide sequence;
Y₁ is O;
Y₂ is O;
Y₄ is CO, SO₂, P(O)O, CH₂—O—C(O), CH₂—NH—C(O), CH₂—NH—SO₂, or CH₂;
n₂ is 1, 2, 3, 4, 5, or 6;
each n₁, n₃, n₄ and n₅ is independently 0, 1, 2, 3, 4, 5, or 6.
91. An oligonucleotide comprising a moiety of Formula (XVII), or a salt, solvate, or hydrate thereof:

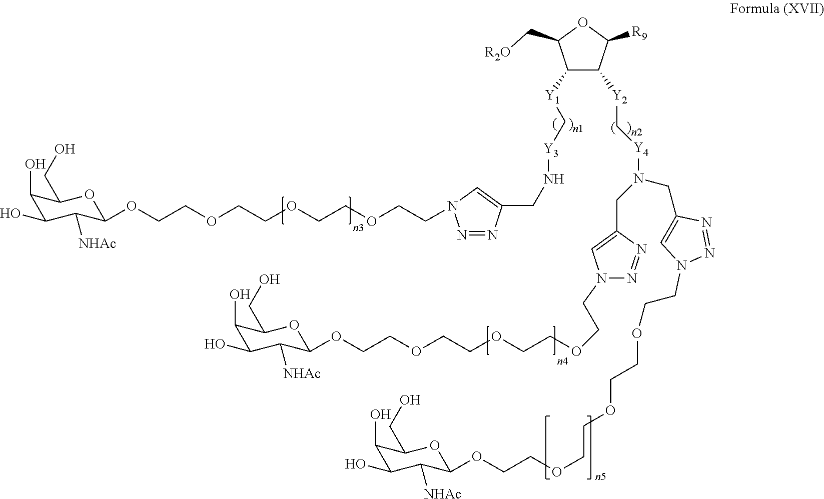

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, or optionally substituted heteroaryl;
R² is a bond;
Y₁ is O;
Y₂ is O;
Y₃ is CO, SO₂, P(O)O, CH₂—O—C(O), CH₂—NH—C(O), CH₂—NH—SO₂, or CH₂;
Y₄ is CO, SO₂, P(O)O, CH₂—O—C(O), CH₂—NH—C(O), CH₂—NH—SO₂, or CH₂;
n₂ is 1, 2, 3, 4, 5, or 6;
each n₁, n₃, n₄ and n₅ is independently 0, 1, 2, 3, 4, 5, or 6.
92. An oligonucleotide comprising a moiety of Formula (XVIII), or a salt, solvate, or hydrate thereof:

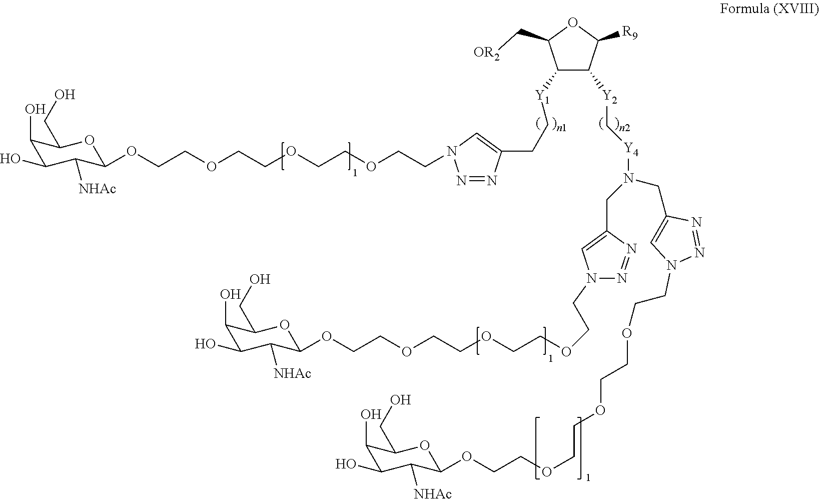

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, or optionally substituted heteroaryl;
R² is a bond;
Y₁ is O;
Y₂ is O;
Y₄ is CO, SO₂, P(O)O, CH₂—O—C(O), CH₂—NH—C(O), CH₂—NH—SO₂, or CH₂;
n₂ is 1, 2, 3, 4, 5, or 6;
each n₁, n₃, n₄ and n₅ is independently 0, 1, 2, 3, 4, 5, or 6.
93. The compound of any of embodiments 80-82 or 85-92, wherein R⁹ is H, adenine, guanine, thymine, cytosine, or uracil, or adenine, guanine, thymine, cytosine, or uracil, each comprising a Protecting Group (PG).
94. The compound of any of embodiments 80-82 or 85-92, wherein R⁹ is a modified nucleobase.
95. The compound of any of embodiments 80-82 or 85-92, wherein R⁹ is optionally substituted alkyl, optionally substituted aryl, or optionally substituted heteroaryl.
96. A composition comprising a compound of any of embodiments 80-95, and a pharmaceutically acceptable carrier.
97. A method for modulating protein function in a subject, comprising administration of a compound of any of embodiments 80-95 or composition of embodiment 96 to the subject.
98. A method for treating or ameliorating a disease, disorder, or symptom thereof in a subject, comprising administration of a compound of any of embodiments 80-95 or composition of embodiment 96 to the subject.
99. The method of embodiment 98, wherein the disease, disorder, or symptom thereof is a liver disease, disorder, or symptom thereof.
100. A method for making a compound of any of embodiments 80-95 comprising one or more compounds and chemical transformations described herein, including Examples 1-4.
101. A compound of Formula (XIX), or a salt, solvate, or hydrate thereof:

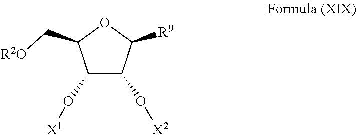

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, or optionally substituted heteroaryl;
R² is H, Protecting Group (PG), or

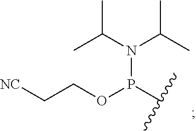 [SMILES: *P(OCCC#N)N(C(C)C)C(C)C]

X¹ is

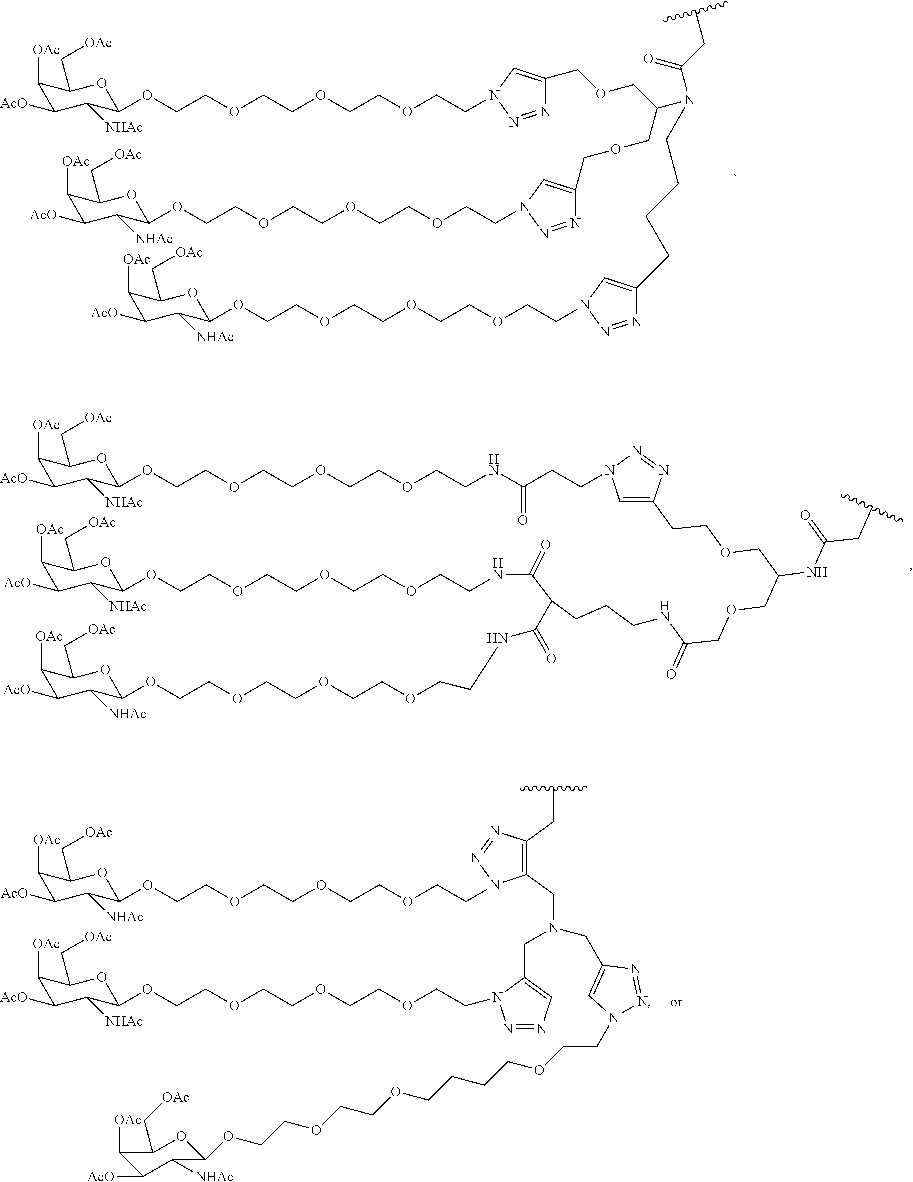

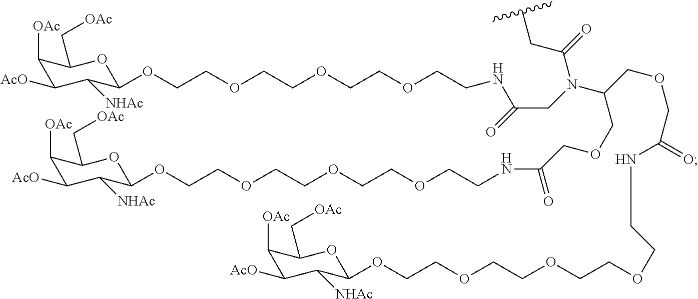 [SMILES: *CC(=O)N(CC(=O)NCCOCCOCCOCCOC1OC(COC(C)=O)C(OC(C)=O)C(OC(C)=O)C1NC(C)=O)C(COCC(=O)NCCOCCOCCOCCOC1OC(COC(C)=O)C(OC(C)=O)C(OC(C)=O)C1NC(C)=O)COCC(=O)NCCOCCOCCOCCOC1OC(COC(C)=O)C(OC(C)=O)C(OC(C)=O)C1NC(C)=O]

X² is H, alkyl, or Protecting Group (PG).
102. A compound of Formula (XX), or a salt, solvate, or hydrate thereof:

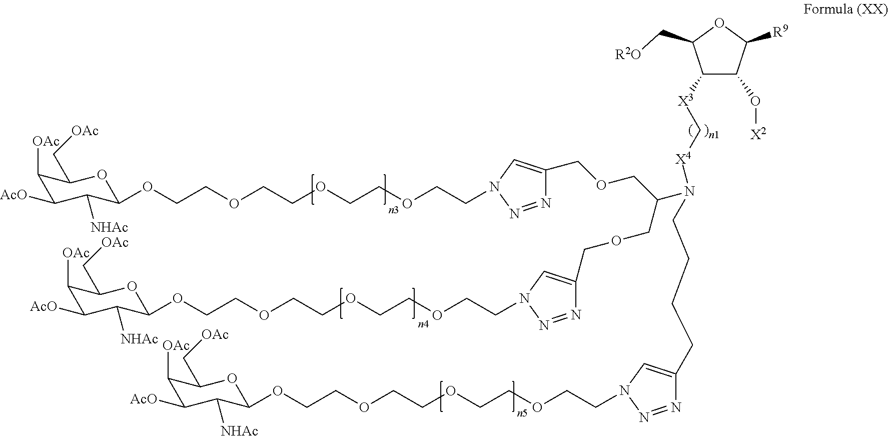

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, or optionally substituted heteroaryl;
R² is H, Protecting Group (PG), or

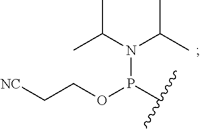 [SMILES: *P(OCCC#N)N(C(C)C)C(C)C]

X² is H, alkyl, or Protecting Group (PG);
X³ is O;
X⁴ is CO, SO₂, P(O)O, CH₂—O—C(O), CH₂—NH—C(O), CH₂—NH—SO₂, or CH₂; and
each n₁, n₃, n₄ and n₅ is independently 0, 1, 2, 3, 4, 5, or 6.
103. A compound of Formula (XXI), or a salt, solvate, or hydrate thereof:

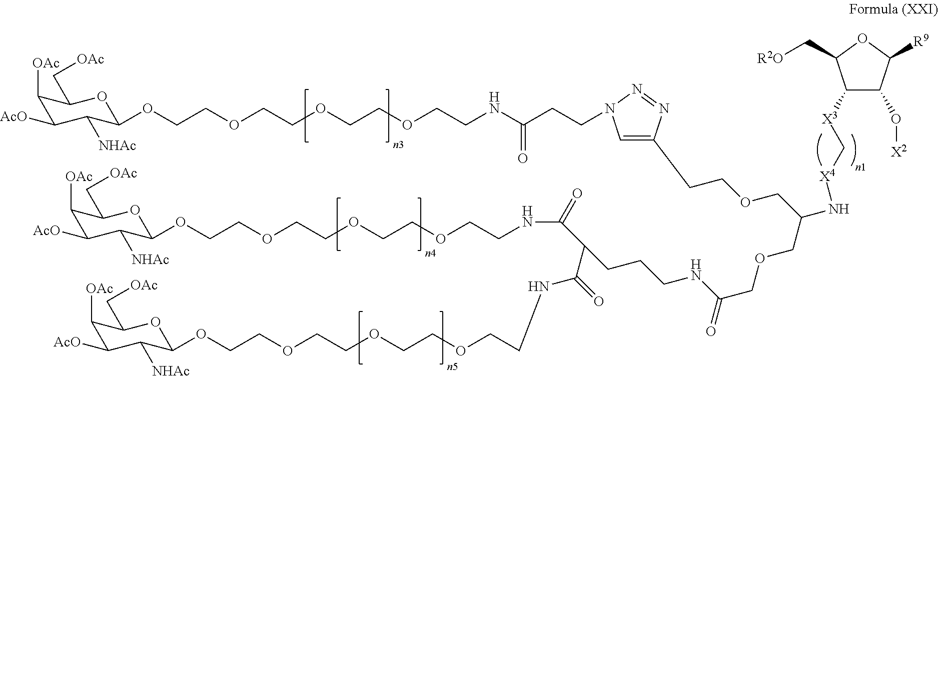

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, or optionally substituted heteroaryl;
R² is H, Protecting Group (PG), or

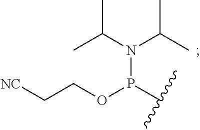 [SMILES: *P(OCCC#N)N(C(C)C)C(C)C]

X² is H, alkyl, or Protecting Group (PG);
X³ is O;
X⁴ is CO, SO₂, P(O)O, CH₂—O—C(O), CH₂—NH—C(O), CH₂—NH—SO₂, or CH₂; and
each n₁, n₃, n₄ and n₅ is independently 0, 1, 2, 3, 4, 5, or 6.
104. A compound of Formula (XXII), or a salt, solvate, or hydrate thereof:

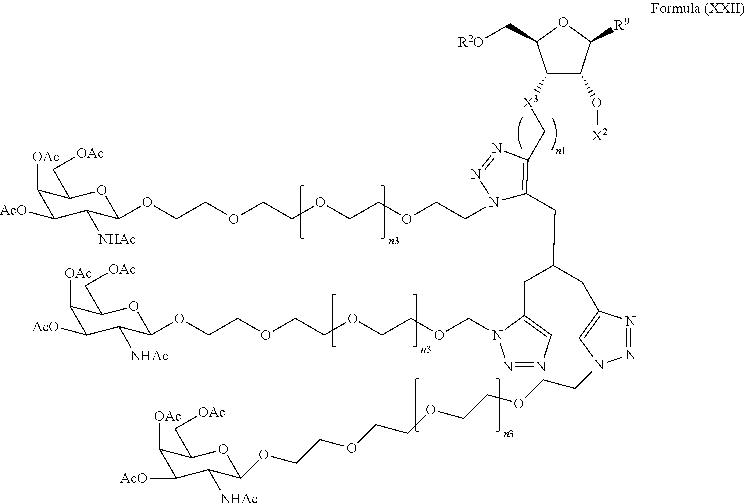

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, or optionally substituted heteroaryl;
R² is H, Protecting Group (PG), or

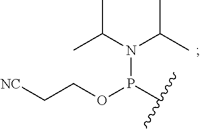 [SMILES: *P(OCCC#N)N(C(C)C)C(C)C]

X² is H, alkyl, or Protecting Group (PG);
X³ is O;
each n₁, n₃, n₄ and n₅ is independently 0, 1, 2, 3, 4, 5, or 6.
105. A compound of Formula (XXIII), or a salt, solvate, or hydrate thereof:

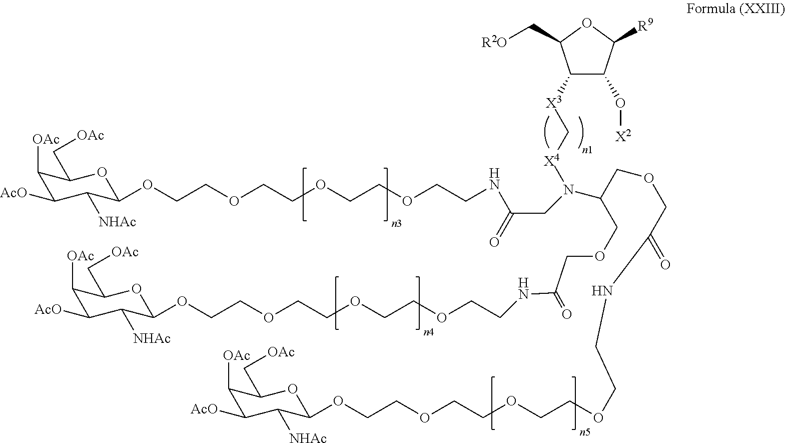

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, or optionally substituted heteroaryl;
R² is H, Protecting Group (PG), or

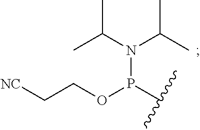 [SMILES: *P(OCCC#N)N(C(C)C)C(C)C]

X² is H, alkyl, or Protecting Group (PG);
X³ is O;
X⁴ is CO, SO₂, P(O)O, CH₂—O—C(O), CH₂—NH—C(O), CH₂—NH—SO₂, or CH₂; and
each n₁, n₃, n₄ and n₅ is independently 0, 1, 2, 3, 4, 5, or 6.
106. The compound of embodiment 102 that is:

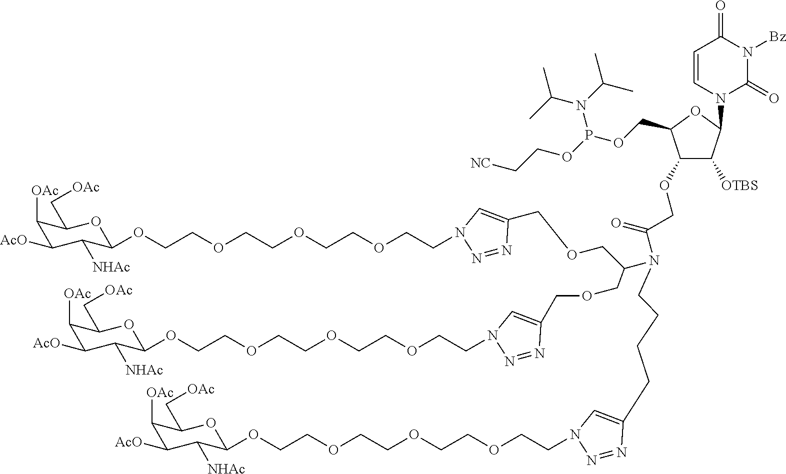

or a salt, solvate, or hydrate thereof.
107. The compound of embodiment 103 that is:

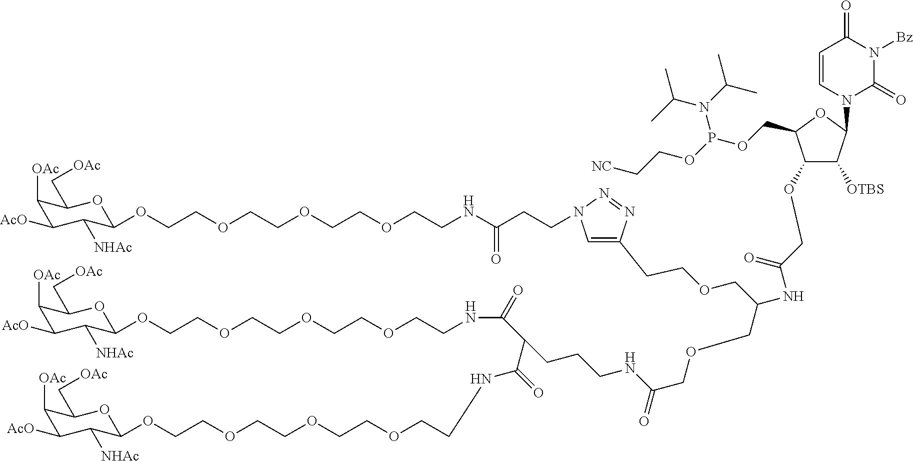

or a salt, solvate, or hydrate thereof.
108. The compound of embodiment 104 that is:

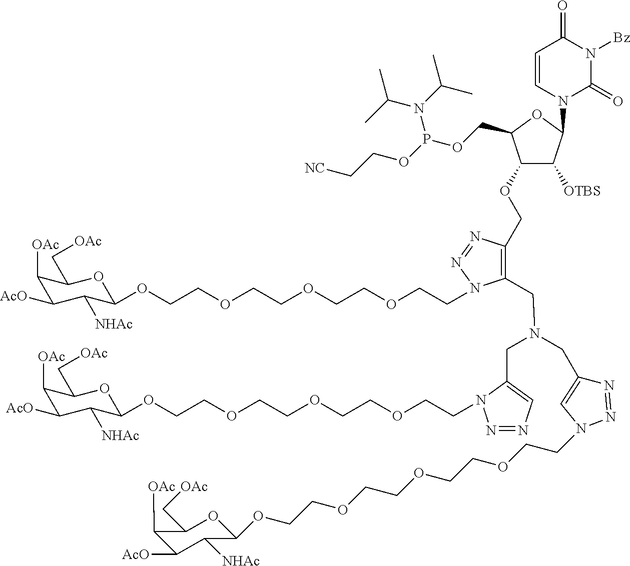

or a salt, solvate, or hydrate thereof.
109. The compound of embodiment 105 that is:

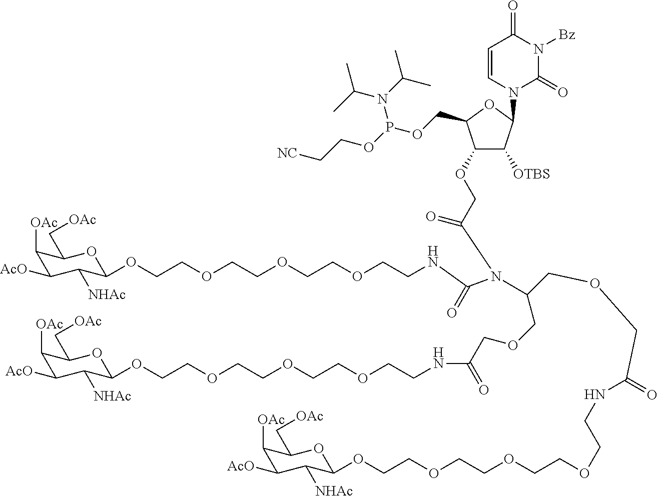

or a salt, solvate, or hydrate thereof.
110. A compound of Formula (XXIV), or a salt, solvate, or hydrate thereof:

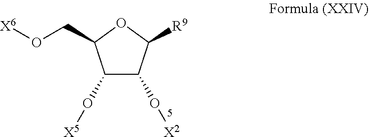

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, or optionally substituted heteroaryl;
X² is H, alkyl, or Protecting Group (PG);
X⁵ is H, Protecting Group (PG), or

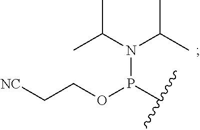 [SMILES: *P(OCCC#N)N(C(C)C)C(C)C]

and
X⁶ is

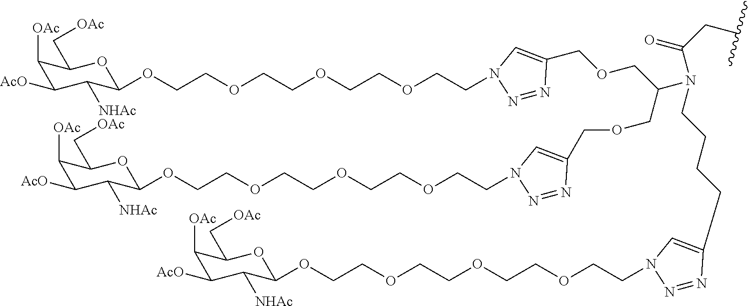 [SMILES: *CC(=O)N(CCCCc1cn(CCOCCOCCOCCOC2OC(COC(C)=O)C(OC(C)=O)C(OC(C)=O)C2NC(C)=O)nn1)C(COCc1cn(CCOCCOCCOCCOC2OC(COC(C)=O)C(OC(C)=O)C(OC(C)=O)C2NC(C)=O)nn1)COCc1cn(CCOCCOCCOCCOC2OC(COC(C)=O)C(OC(C)=O)C(OC(C)=O)C2NC(C)=O)nn1]

111. A compound of Formula (XXV), or a salt, solvate, or hydrate thereof:

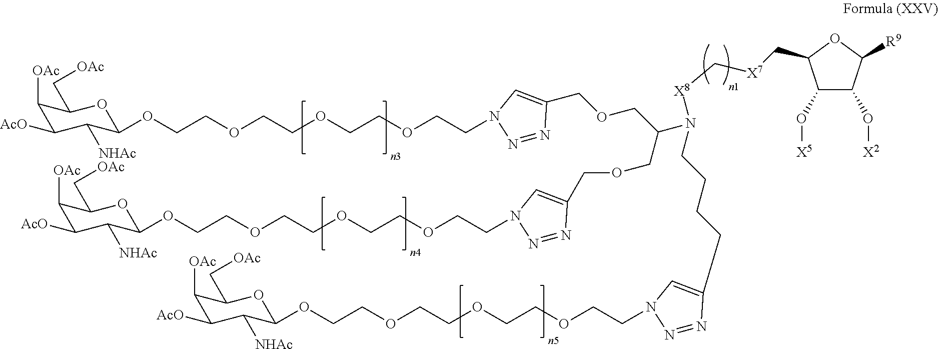

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, or optionally substituted heteroaryl;
X² is H, alkyl, or Protecting Group (PG);
X⁵ is H, Protecting Group (PG), or

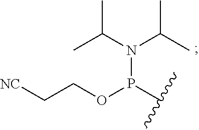 [SMILES: *P(OCCC#N)N(C(C)C)C(C)C]

X⁷ is O;
X⁸ is CO, SO₂, P(O)O, CH₂—O—C(O), CH₂—NH—C(O), CH₂—NH—SO₂, or CH₂; and
each n₁, n₃, n₄ and n₅ is independently 0, 1, 2, 3, 4, 5, or 6.
112. The compound of embodiment 111 that is:

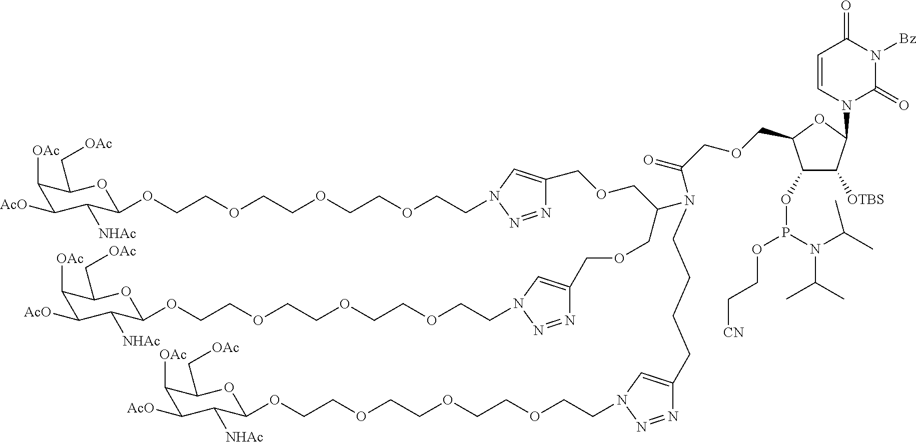

or a salt, solvate, or hydrate thereof.
113. An oligonucleotide comprising the compound of any one of embodiments 101-112.
114. A composition comprising a compound of any one of embodiments 101-112, or an oligonucleotide of embodiment 113, and a pharmaceutically acceptable carrier.
115. A method for modulating protein function in a subject comprising administration of a compound of any one of embodiments 101-112, an oligonucleotide of embodiment 113, or a composition of embodiment 114 to the subject.
116. A method for treating or ameliorating a disease, disorder, or symptom thereof in a subject, comprising administration of a compound of any one of embodiments 101-112, an oligonucleotide of embodiment 113, or a composition of embodiment 114.
117. The method of embodiment 116, wherein the disease, disorder, or symptom thereof is a liver disease, disorder, or symptom thereof.
118. A method for making a compound of any one of embodiments 101-112 comprising one or more compounds and chemical transformations described herein, including Example 5.
119. A compound of Formula (I), or a salt, solvate, or hydrate thereof:

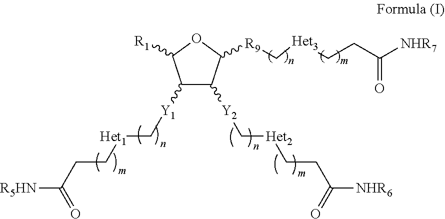

wherein:
each n is independently 1, 2, 3, 4, or 5;
each m is independently 0, 1, 2, 3, 4, 5, or 6;
each o is independently 0, 1, 2, 3, 4, 5, or 6;
each Y₁ is independently O, CH(R^{a}), S, S(═O), S(═O)₂, NH, substituted N group, NHC(═O), C(═O)NH, P(═O)₂—O—, P(═O)(═S)—O, P(═S)₂—O, —O—P(═O)₂—O—, —O—P(═O)(═S)—O—, —O—P(═S)₂—O—, —O—P(═O)₂—, —O—P(═O)(═S)—, —O—P(═S)₂—;
each Y₂ is independently O, CH(R^{b}), S, S(═O), S(═O)₂, NH, substituted N group, NHC(═O), C(═O)NH, P(═O)₂—O—, P(═O)(═S)—O, P(═S)₂—O, —O—P(═O)₂—O—, —O—P(═O)(═S)—O—, —O—P(═S)₂—O—, —O—P(═O)₂—, —O—P(═O)(═S)—, —O—P(═S)₂—;
each of Het₁, Het₂, and Het₃ is independently optionally substituted heteroaryl or optionally substituted heterocyclyl;
each R₁ is independently alkyl-O-phosphoramidite, optionally substituted alkenyl phosphoramidite, optionally substituted alkynyl phosphoramidite, OH, NH₂, NHR^{a}, N₃, C(═O)OH, C(═O)X, CN, SH, SSH, SO₂X, C(═O)NHNH₂, NHNH₂, C(═S)NHNH₂, C(═S)NH₂, NHOH, C(═O)CH₂X, malonyl, alkyl, alkenyl, dienyl, alkynyl, heteroalkyl, —OP(═S)X, —C(═O)H, —C(═O)R^{a}, —N═C═O, —N═C═NR^{a}, —N═C═S, CHX, —OP(═O)OH, phosphane, alkoxyphosphane, —C(R^{a})₂, a Michael acceptor, a protein, or a therapeutic agent for modulating hepatocytes or treating liver disease, wherein X is a leaving group;
each R₅, R₆, and R₇ is independently

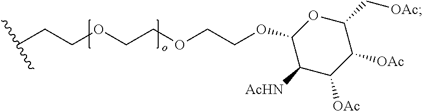

R₉ is optionally substituted heterocyclyl;
each R^{a} is independently H, alkyl, halo, OR^{c}, or SR^{c};
each R^{b} is independently H, alkyl, halo, OR^{c}, or SR^{c}; and
each R^{c} is independently H or alkyl.
120. The compound of embodiment 119, or a salt, solvate, or hydrate thereof, wherein R₉ is an optionally substituted nitrogen-containing heterocyclyl.
121. The compound of embodiment 119, or a salt, solvate, or hydrate thereof, wherein R₉ is an optionally substituted pyrimidinyl.
122. The compound of embodiment 119, or a salt, solvate, or hydrate thereof, wherein R₉ is a 4H-1λ², 3λ²-pyrimidine-2,4-dione.
123. The compound of embodiment 119, or a salt, solvate, or hydrate thereof, wherein R₉ is

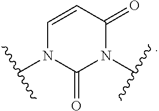 [SMILES: *n1ccc(=O)n(*)c1=O]

124. The compound of any of embodiments 119-123, or a salt, solvate, or hydrate thereof, wherein each of Het₁, Het₂, and Het₃ is independently optionally substituted nitrogen-containing heterocyclyl or optionally substituted nitrogen-containing heteroaryl.
125. The compound of any of embodiments 119-123, or a salt, solvate, or hydrate thereof, wherein each of Het₁, Het₂, and Het₃ is independently optionally substituted 1, 2, 3-triazolyl.
126. The compound of any of embodiments 119-123, or a salt, solvate, or hydrate thereof, wherein each of Het₁, Het₂, and Het₃ is independently optionally substituted 1λ²,2,3-triazol-4-yl.
127. The compound of any of embodiments 119-123, or a salt, solvate, or hydrate thereof, wherein each of Het₁, Het₂, and Het₃ is independently

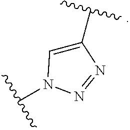 [SMILES: *c1cn(*)nn1]

128. The compound of any of embodiments 119-127, or a salt, solvate, or hydrate thereof, wherein two of R₅, R₆, and R₇ are each the same.
129. The compound of any of embodiments 119-127, or a salt, solvate, or hydrate thereof, wherein R₅, and R₆ are each the same.
130. The compound of any of embodiments 119-127, or a salt, solvate, or hydrate thereof, wherein three of R₅, R₆, and R₇ are each the same.
131. The compound of any of embodiments 119-130, or a salt, solvate, or hydrate thereof, wherein o is the same in each of R₅, R₆, and R₇.
132. The compound of any of embodiments 119-130, or a salt, solvate, or hydrate thereof, wherein o is the same in each of R₅ and R₆.
133. The compound of any of embodiments 119-132, or a salt, solvate, or hydrate thereof, wherein R₁ is alkyl-O-phosphonamidite, optionally substituted alkenyl phosphonamidite, or optionally substituted alkynyl phosphonamidite.
134. The compound of any of embodiments 119-132, or a salt, solvate, or hydrate thereof, wherein R₁ is

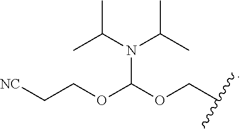 [SMILES: *COC(OCCC#N)N(C(C)C)C(C)C]

135. A compound of Formula (II), or a salt, solvate, or hydrate thereof:

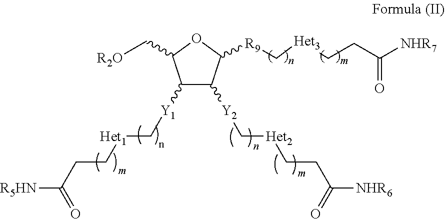

wherein,
each n is independently 1, 2, 3, 4, or 5;
each m is independently 0, 1, 2, 3, 4, 5, or 6;
each o is independently 0, 1, 2, 3, 4, 5, or 6;
each Y₁ is independently O, CH(R^{a}), S, S(═O), S(═O)₂, NH, substituted N group, NHC(═O), C(═O)NH, P(═O)₂—O—, P(═O)(═S)—O, P(═S)₂—O, —O—P(═O)₂—O—, —O—P(═O)(═S)—O—, —O—P(═S)₂—O—, —O—P(═O)₂—, —O—P(═O)(═S)—, —O—P(═S)₂—;
each Y₂ is independently O, CH(R^{b}), S, S(═O), S(═O)₂, NH, substituted N group, NHC(═O), C(═O)NH, P(═O)₂—O—, P(═O)(═S)—O, P(═S)₂—O, —O—P(═O)₂—O—, —O—P(═O)(═S)—O—, —O—P(═S)₂—O—, —O—P(═O)(═S)—, —O—P(═S)₂—;
each of Het₁, Het₂, and Het₃ is independently optionally substituted heteroaryl or optionally substituted heterocyclyl;
each R₅, R₆, and R₇ is independently

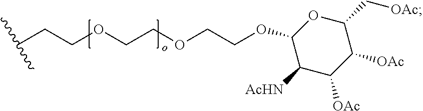

R₉ is optionally substituted heterocyclyl; and
R² is H, Protecting Group (PG),

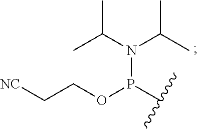 [SMILES: *P(OCCC#N)N(C(C)C)C(C)C]

each R^{a} is independently H, alkyl, halo, OR^{c}, or SR^{c};
each R^{b} is independently H, alkyl, halo, OR^{c}, or SR^{c}; and
each R^{c} is independently H or alkyl.
136. The compound of embodiment 135, or a salt, solvate, or hydrate thereof, wherein R² is H.
137. The compound of embodiment 135, or a salt, solvate, or hydrate thereof, wherein R² is Protecting Group (PG).
138. The compound of embodiment 135, or a salt, solvate, or hydrate thereof, wherein R² is

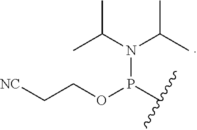 [SMILES: *P(OCCC#N)N(C(C)C)C(C)C]

139. The compound of any of embodiments 135-138, or a salt, solvate, or hydrate thereof, wherein R₉ is an optionally substituted nitrogen-containing heterocyclyl.
140. The compound of any of embodiments 135-138, or a salt, solvate, or hydrate thereof, wherein R₉ is an optionally substituted dihydropyrimidinyl, optionally substituted tetrahydropyrimidinyl, or optionally substituted hexahydropyrimidinyl.
141. The compound of any of embodiments 135-138, or a salt, solvate, or hydrate thereof, wherein R₉ is a 4H-1λ²,3λ²-pyrimidine-2,4-dione.
142. The compound of any of embodiments 135-138, or a salt, solvate, or hydrate thereof, wherein R₉ is

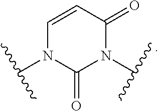 [SMILES: *n1ccc(=O)n(*)c1=O]

143. The compound of any of embodiments 135-142, or a salt, solvate, or hydrate thereof, wherein each of Het₁, Het₁, and Het₃ is independently optionally substituted nitrogen-containing heteroaryl.
144. The compound of any of embodiments 135-142, or a salt, solvate, or hydrate thereof, wherein each of Het₁, Het₂, and Het₃ is independently optionally substituted 1, 2, 3-triazolyl.
145. The compound of any of embodiments 135-142, or a salt, solvate, or hydrate thereof, wherein each of Het₁, Het₂, and Het₃ is independently optionally substituted 1λ²,2,3-triazol-4-yl.
146. The compound of any of embodiments 135-142, or a salt, solvate, or hydrate thereof, wherein each of Het₁, Het₂, and Het₃ is independently

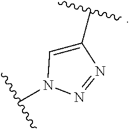 [SMILES: *c1cn(*)nn1]

147. The compound of any of embodiments 135-146, or a salt, solvate, or hydrate thereof, wherein two of R₅, R₆, and R₇ are each the same.
148. The compound of any of embodiments 135-146, or a salt, solvate, or hydrate thereof, wherein R₅, and R₆ are each the same.
149. The compound of any of embodiments 135-146, or a salt, solvate, or hydrate thereof, wherein three of R₅, R₆, and R₇ are each the same.
150. The compound of any of embodiments 135-146, or a salt, solvate, or hydrate thereof, wherein o is the same in each of R₅, R₆, and R₇.
151. The compound of any of embodiments 135-146, or a salt, solvate, or hydrate thereof, wherein o is the same in each of R₅ and R₆.
152. The compound of any one of embodiments 135-151, wherein the compound is of Formula (II-a):

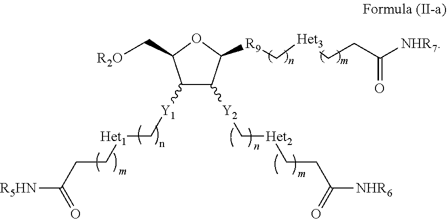

153. A modified oligonucleotide comprising a moiety of Formula (III), or a salt, solvate, or hydrate thereof:

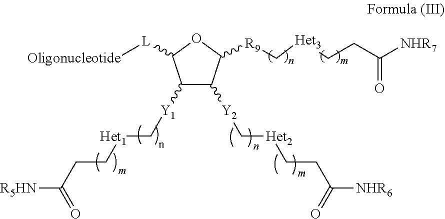

wherein:
each n is independently 1, 2, 3, 4, or 5;
each m is independently 0, 1, 2, 3, 4, 5, or 6;
each o is independently 0, 1, 2, 3, 4, 5, or 6;
each Y₁ is independently O, CH(R^{a}), S, S(═O), S(═O)₂, NH, substituted N group, NHC(═O), C(═O)NH, P(═O)₂—O—, P(═O)(═S)—O, P(═S)₂—O, —O—P(═O)₂—O—, —O—P(═O)(═S)—O—, —O—P(═S)₂—O—, —O—P(═O)(═S)—, —O—P(═S)₂—;
each Y₂ is independently O, CH(R^{b}), S, S(═O), S(═O)₂, NH, substituted N group, NHC(═O), C(═O)NH, P(═O)₂—O—, P(═O)(═S)—O, P(═S)₂—O, —O—P(═O)₂—O—, —O—P(═O)(═S)—O—, —O—P(═S)₂—O—, —O—P(═O)₂—, —O—P(═O)(═S)—, —O—P(═S)₂—;
each of Het₁, Het₂, and Het₃ is independently optionally substituted heteroaryl or optionally substituted heterocyclyl;
each R₅, R₆, and R₇ is independently

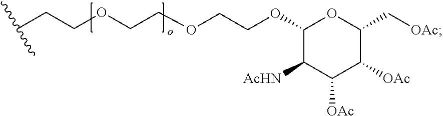

and
R₉ is optionally substituted heterocyclyl;
L is bond, O, CH₂, S, S(═O), S(═O)₂, NH, substituted N group, NHC(═O), C(═O)NH, alkyl, alkenyl, dienyl, alkynyl, heteroalkyl, or a thiol-Michael adduct;
each R^{a} is independently H, alkyl, halo, OR^{c}, or SR^{c};
each R^{b} is independently H, alkyl, halo, OR^{c}, or SR^{c}; and
each R^{c} is independently H or alkyl.
154. The compound of embodiment 153, or a salt, solvate, or hydrate thereof, wherein R₉ is an optionally substituted nitrogen-containing heterocyclyl.
155. The compound of embodiment 153, or a salt, solvate, or hydrate thereof, wherein R₉ is an optionally substituted dihydropyrimidinyl, optionally substituted tetrahydropyrimidinyl, or optionally substituted hexahydropyrimidinyl.
156. The compound of embodiment 153, or a salt, solvate, or hydrate thereof, wherein R₉ is a 4H-1λ²,3λ²-pyrimidine-2,4-dione.
157. The compound of embodiment 153, or a salt, solvate, or hydrate thereof, wherein R₉ is

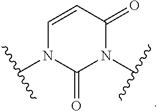 [SMILES: *n1ccc(=O)n(*)c1=O]

158. The compound of any of embodiments 153-157, or a salt, solvate, or hydrate thereof, wherein each of Het₁, Het₂, and Het₃ is independently optionally substituted nitrogen-containing heteroaryl.
159. The compound of any of embodiments 153-157, or a salt, solvate, or hydrate thereof, wherein each of Het₁, Het₂, and Het₃ is independently optionally substituted 1, 2, 3-triazolyl.
160. The compound of any of embodiments 153-157, or a salt, solvate, or hydrate thereof, wherein each of Het₁, Het₂, and Het₃ is independently optionally substituted 1λ²,2,3-triazol-4-yl.
161. The compound of any of embodiments 153-157, or a salt, solvate, or hydrate thereof, wherein each of Het₁, Het₂, and Het₃ is independently

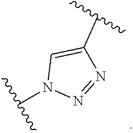 [SMILES: *c1cn(*)nn1]

162. The compound of any of embodiments 153-161, or a salt, solvate, or hydrate thereof, wherein two of R₅, R₆, and R₇ are each the same.
163. The compound of any of embodiments 153-161, or a salt, solvate, or hydrate thereof, wherein R₅, and R₆ are each the same.
164. The compound of any of embodiments 153-161, or a salt, solvate, or hydrate thereof, wherein three of R₅, R₆, and R₇ are each the same.
165. The compound of any of embodiments 153-164, or a salt, solvate, or hydrate thereof, wherein o is the same in each of R₅, R₆, and R₇.
166. The compound of any of embodiments 153-164, or a salt, solvate, or hydrate thereof, wherein o is the same in each of R₅ and R₆.
167. The modified oligonucleotide of any one of embodiments 153-166, wherein the oligonucleotide comprises an siRNA, an miRNA, an ADAR recruiting molecule, an ADAR targeting molecule, a guide RNA, or an antisense nucleic acid.
168. The compound of any one of embodiments 119-167, or salt, solvate, or hydrate thereof, wherein for use as a reagent in a chemical reaction.
169. An oligonucleotide comprising a moiety of Formula (IV), or salt, solvate, or hydrate thereof:

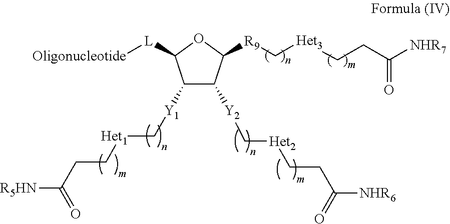

wherein:
each n is independently 1, 2, 3, 4, or 5;
each m is independently 0, 1, 2, 3, 4, 5, or 6;
each o is independently 0, 1, 2, 3, 4, 5, or 6;
each Y₁ is independently O, CH(R^{a}), S, S(═O), S(═O)₂, NH, substituted N group, NHC(═O), C(═O)NH, P(═O)₂—O—, P(═O)(═S)—O, P(═S)₂—O, —O—P(═O)₂—O—, —O—P(═O)(═S)—O—, —O—P(═S)₂—O—, —O—P(═O)₂—, —O—P(═O)(═S)—, —O—P(═S)₂—;
each Y₂ is independently O, CH(R^{b}), S, S(═O), S(═O)₂, NH, substituted N group, NHC(═O), C(═O)NH, P(═O)₂—O—, P(═O)(═S)—O, P(═S)₂—O, —O—P(═O)₂—O—, —O—P(═O)(═S)—O—, —O—P(═S)₂—O—, —O—P(═O)₂—, —O—P(═O)(═S)—, —O—P(═S)₂—;
each of Het₁, Het₂, and Het₃ is independently optionally substituted heteroaryl or optionally substituted heterocyclyl;
each R₅, R₆, and R₇ is independently

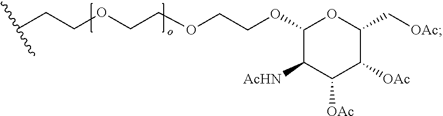

R₉ is optionally substituted heterocyclyl;
L is bond, O, CH₂, S, S(═O), S(═O)₂, NH, substituted N group, NHC(═O), C(═O)NH, alkyl, alkenyl, dienyl, alkynyl, heteroalkyl, or a thiol-Michael adduct;
each R^{a} is independently H, alkyl, halo, OR^{c}, or SR^{c};
each R^{b} is independently H, alkyl, halo, OR^{c}, or SR^{c}; and
each R^{c} is independently H or alkyl.
170. The oligonucleotide of embodiment 169, wherein the oligonucleotide comprises an siRNA, an miRNA, an ADAR recruiting molecule, an ADAR targeting molecule, a guide RNA, or an antisense nucleic acid.
171. The oligonucleotide of embodiment 169, or a salt, solvate, or hydrate thereof, wherein R₉ is an optionally substituted nitrogen-containing heterocyclyl.
172. The oligonucleotide of embodiment 169, or a salt, solvate, or hydrate thereof, wherein R₉ is an optionally substituted dihydropyrimidinyl, optionally substituted tetrahydropyrimidinyl, or optionally substituted hexahydropyrimidinyl.
173. The oligonucleotide of embodiment 169, or a salt, solvate, or hydrate thereof, wherein R₉ is a 4H-1λ²,3λ²-pyrimidine-2,4-dione.
174. The oligonucleotide of embodiment 169, or a salt, solvate, or hydrate thereof, wherein R₉ is

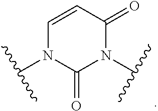 [SMILES: *n1ccc(=O)n(*)c1=O]

175. The oligonucleotide of any of embodiments 169-174, or a salt, solvate, or hydrate thereof, wherein each of Het₁, Het₂, and Het₃ is independently optionally substituted nitrogen-containing heteroaryl.
176. The oligonucleotide of any of embodiments 169-174, or a salt, solvate, or hydrate thereof, wherein each of Het₁, Het₂, and Het₃ is independently optionally substituted 1, 2, 3-triazolyl.
177. The oligonucleotide of any of embodiments 169-174, or a salt, solvate, or hydrate thereof, wherein each of Het₁, Het₂, and Het₃ is independently optionally substituted 1λ²,2,3-triazol-4-yl.
178. The oligonucleotide of any of embodiments 169-174, or a salt, solvate, or hydrate thereof, wherein each of Het₁, Het₂, and Het₃ is independently

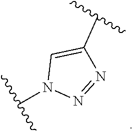 [SMILES: *c1cn(*)nn1]

179. The oligonucleotide of any of embodiments 169-178, or a salt, solvate, or hydrate thereof, wherein two of R₅, R₆, and R₇ are each the same.
180. The oligonucleotide of any of embodiments 169-178, or a salt, solvate, or hydrate thereof, wherein R₅, and R₆ are each the same.
181. The oligonucleotide of any of embodiments 169-178, or a salt, solvate, or hydrate thereof, wherein three of R₅, R₆, and R₇ are each the same.
182. The oligonucleotide of any of embodiments 169-181, or a salt, solvate, or hydrate thereof, wherein o is the same in each of R₅, R₆, and R₇.
183. The oligonucleotide of any of embodiments 169-81, or a salt, solvate, or hydrate thereof, wherein o is the same in each of R₅ and R₆.
184. A compound, or a salt, solvate, or hydrate thereof, that is Example 1.
185. A compound, or a salt, solvate, or hydrate thereof, that is one of INT-1, INT-2, INT-3, INT-4, INT-5, INT-6, or INT-7.
186. A compound, or a salt, solvate, or hydrate thereof, that is one of INT-5 or INT-6.
187. A compound, or a salt, solvate, or hydrate thereof, that is INT-7.
188. A composition comprising a compound of any of embodiments 119-187, and a pharmaceutically acceptable carrier.
189. A method for modulating protein function in a subject, comprising administration of a compound of any of embodiments 119-187 to the subject.
190. A method for treating or ameliorating a disease, disorder, or symptom thereof in a subject, comprising administration of a compound of any of embodiments 119-187 to the subject.
191. A method of making a compound of Formula (V), or salt, solvate, or hydrate thereof:

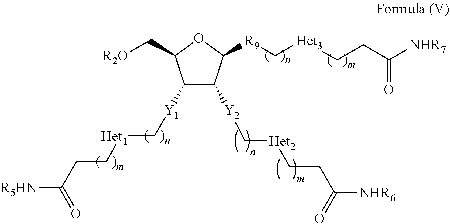

wherein:
each n is independently 1, 2, 3, 4, or 5;
each m is independently 0, 1, 2, 3, 4, 5, or 6;
each o is independently 0, 1, 2, 3, 4, 5, or 6;
each Y₁ is independently O, CH(R^{a}), S, S(═O), S(═O)₂, NH, substituted N group, NHC(═O), C(═O)NH, P(═O)₂—O—, P(═O)(═S)—O, P(═S)₂—O, —O—P(═O)₂—O—, —O—P(═O)(═S)—O—, —O—P(═S)₂—O—, —O—P(═O)₂—, —O—P(═O)(═S)—, —O—P(═S)₂—;
each Y₂ is independently O, CH(R^{b}), S, S(═O), S(═O)₂, NH, substituted N group, NHC(═O), C(═O)NH, P(═O)₂—O—, P(═O)(═S)—O, P(═S)₂—O, —O—P(═O)₂—O—, —O—P(═O)(═S)—O—, —O—P(═S)₂—O—, —O—P(═O)₂—, —O—P(═O)(═S)—, —O—P(═S)₂—;
each of Het₁, Het₂, and Het₃ is independently optionally substituted heteroaryl or optionally substituted heterocyclyl;
each R₅, R₆, and R₇ is independently

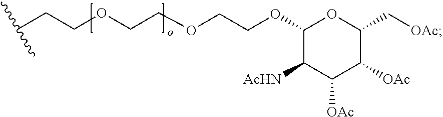

R₉ is optionally substituted heterocyclyl;
wherein R² is

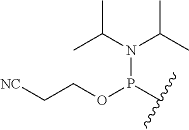 [SMILES: *P(OCCC#N)N(C(C)C)C(C)C]

or a therapeutic agent for modulating hepatocytes or treating liver disease;
each R^{a} is independently H, alkyl, halo, OR^{c}, or SR^{c};
each R^{b} is independently H, alkyl, halo, OR^{c}, or SR^{c}; and
each R^{c} is independently H or alkyl;
comprising reacting a compound of Formula (V) wherein R₂ is H (and remaining variables are as described above) with a reactant of the formula

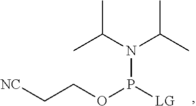

wherein LG is a leaving group (e.g., halides, —OS(O)₂R, —OS(O)₂OR, —OC(O)R, etc.; (where R is alkyl, arylalkyl, aryl, each of which may be optionally substituted)).
192. The method of embodiment 191, wherein the reactant is 3-((chloro(diisopropylamino)phosphanyl)oxy)propanenitrile.
193. A method of making a compound of formula INT-7, comprising reacting a compound of formula INT-6 with a compound of INT-3.
194. The method of embodiment 193, further comprising a copper catalyst.
195. The method of embodiment 193, further comprising CuSO₄·5 H₂O.
196. The method of embodiment 195, further comprising sodium ascorbate.
197. The compound of any of embodiments 119-133, or a salt, solvate, or hydrate thereof, wherein each R₁ is independently alkyl-O-phosphonamidite, optionally substituted alkenyl phosphonamidite, or optionally substituted alkynyl phosphonamidite.
198. The compound of any of embodiments 119-133, or a salt, solvate, or hydrate thereof, wherein each R₁ is independently OH, NH₂, NHR^{a}, N₃, C(═O)OH, C(═O)X, CN, SH, SSH, SO₂X, C(═O)NHNH₂, NHNH₂, C(═S)NHNH₂, C(═S)NH₂, NHOH, C(═O)CH₂X, malonyl, alkyl, alkenyl, dienyl, alkynyl, heteroalkyl, —OP(═S)X, —C(═O)H, —C(═O)R^{a}, —N═C═O, —N═C═NR^{a}, —N═C═S, CHX, —OP(═O)OH, phosphane, alkoxyphosphane, or —C(R^{a})₂, wherein X is a leaving group.
199. The compound of any of embodiments 119-133, or a salt, solvate, or hydrate thereof, wherein each R₁ is independently a Michael acceptor, a protein, or a therapeutic agent for modulating hepatocytes or treating liver disease.
200. A compound of Formula (IX-a), or a salt, solvate, or hydrate thereof:

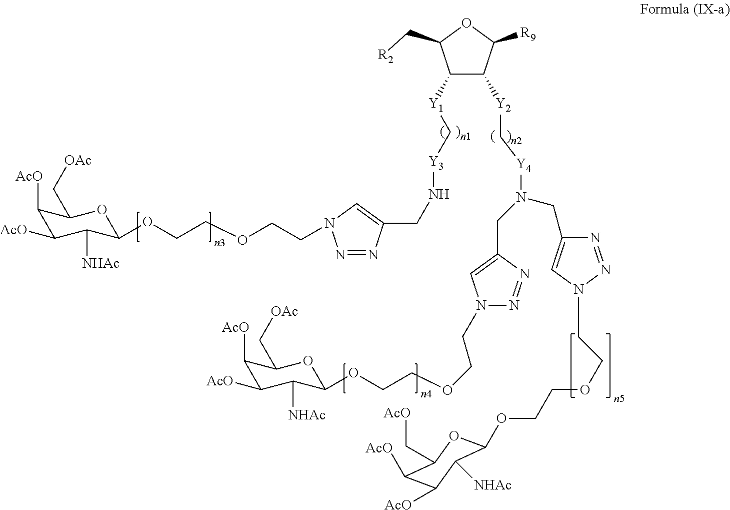

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, optionally substituted heteroaryl, optionally substituted carbocyclyl, optionally substituted heterocyclyl, or a nucleobase isostere;
R² is H, OH, O-Protecting Group (PG),

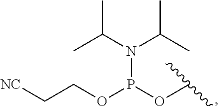 [SMILES: *OP(OCCC#N)N(C(C)C)C(C)C]

a linker, an azide, a carboxylic acid, or an amine;
Y₁ is O, optionally substituted NH, CH₂, or CH₂O;
Y₂ is O, optionally substituted NH, CH₂, or CH₂O;
Y₃ is CO, SO₂, P(O)O, CH₂—O—C(O), CH₂—NH—C(O), CH₂—NH—SO₂, or CH₂;
Y₄ is CO, SO₂, P(O)O, CH₂—O—C(O), CH₂—NH—C(O), CH₂—NH—SO₂, or CH₂;
n₂ is 0, 1, 2, 3, 4, 5, or 6; and
each n₁, n₃, n₄ and n₅ is independently 0, 1, 2, 3, 4, 5, 6, 7, 8, 9, or 10.
201. The compound of embodiment 200, wherein R⁹ is uracil comprising a protecting group.
202. The compound of embodiment 200 or 201, wherein R⁹ is uracil comprising a benzoyl protecting group.
203. The compound of any one of embodiments 200-202, wherein R² is O-Protecting Group (PG).
204. The compound of any one of embodiments 200-203, wherein R² is

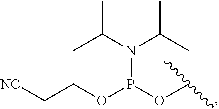 [SMILES: *OP(OCCC#N)N(C(C)C)C(C)C]

205. The compound of any one of embodiments 200-204, wherein Y₁ is O.
206. The compound of any one of embodiments 200-205, wherein Y₂ is O.
207. The compound of any one of embodiments 200-206, wherein both Y₁ and Y₂ are O.
208. The compound of any one of embodiments 200-207, wherein Y₃ is CO.
209. The compound of any one of embodiments 200-208, wherein Y₄ is CO.
210. The compound of any one of embodiments 200-209, wherein Y₃ and Y₄ are CO.
211. The compound of any one of embodiments 200-210, wherein Y₁ and Y₂ are O, and Y₃ and Y₄ are CO.
212. The compound of any one of embodiments 200-211, wherein n₃ is 3.
213. The compound of any one of embodiments 200-212, wherein n₄ is 3.
214. The compound of any one of embodiments 200-213, wherein n₅ is 3.
215. The compound of embodiment 200, wherein the compound is of Formula (IX-b):

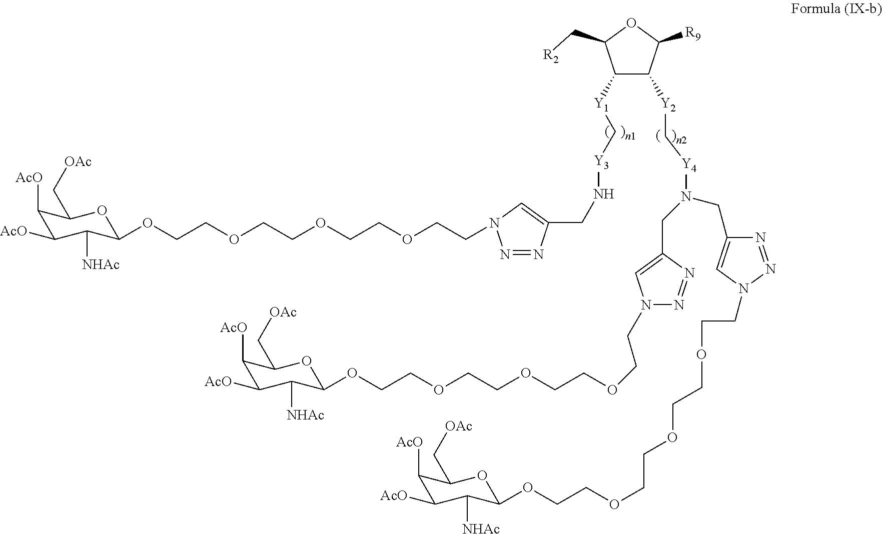

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, optionally substituted heteroaryl, optionally substituted carbocyclyl, optionally substituted heterocyclyl, or a nucleobase isostere;
R² is H, OH, O-Protecting Group (PG),

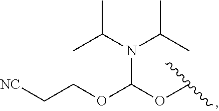 [SMILES: *OC(OCCC#N)N(C(C)C)C(C)C]

a linker, an azide, a carboxylic acid, or an amine;
Y₁ is O, optionally substituted NH, CH₂, or CH₂O;
Y₂ is O, optionally substituted NH, CH₂, or CH₂O;
Y₃ is CO, SO₂, P(O)O, CH₂—O—C(O), CH₂—NH—C(O), CH₂—NH—SO₂, or CH₂;
Y₄ is CO, SO₂, P(O)O, CH₂—O—C(O), CH₂—NH—C(O), CH₂—NH—SO₂, or CH₂;
n₂ is 0, 1, 2, 3, 4, 5, or 6; and
n₁ 0, 1, 2, 3, 4, 5, 6, 7, 8, 9, or 10.
216. A compound of Formula (XV-a), or a salt, solvate, or hydrate thereof:

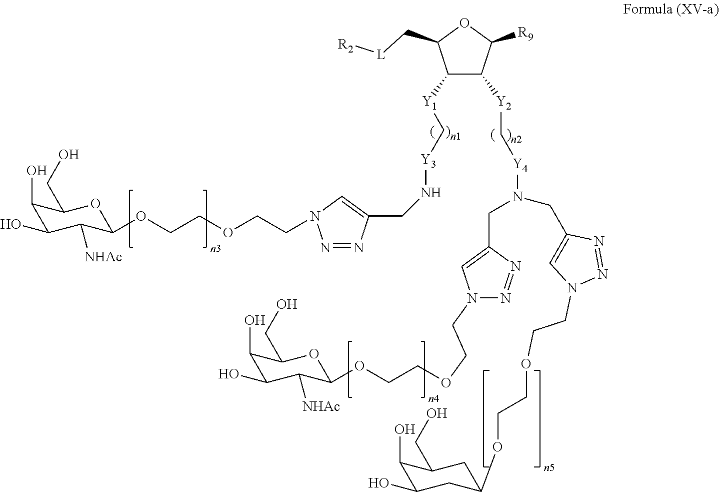

wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, optionally substituted heteroaryl, optionally substituted carbocyclyl, optionally substituted heterocyclyl, or a nucleobase isostere;
L is a bond, a phosphodiester bond, a phosphorothioate bond, a triazole, a tetrazole, an amide, a reverse-amide, a carbamate, a carbonate, urea, alkyl, or heteroalkyl;
R² is an oligonucleotide sequence;
Y₁ is O, CH₂, CH₂O, or optionally substituted NH;
Y₂ is O, CH₂, CH₂O, or optionally substituted NH;
Y₃ is CO, SO₂, P(O)O, CH₂—O—C(O), CH₂—NH—C(O), CH₂—NH—SO₂, or CH₂;
Y₄ is CO, SO₂, P(O)O, CH₂—O—C(O), CH₂—NH—C(O), CH₂—NH—SO₂, or CH₂;
n₂ is 0, 1, 2, 3, 4, 5, or 6; and
each n₁, n₃, n₄ and n₅ is independently 0, 1, 2, 3, 4, 5, 6, 7, 8, 9, or 10.
217. The compound of embodiment 216, wherein Y₁ is O.
218. The compound of embodiment 216 or 217, wherein Y₂ is O.
219. The compound of any one of embodiments 216-218, wherein both Y₁ and Y₂ are O.
220. The compound of any one of embodiments 216-219, wherein Y₃ is CO.
221. The compound of any one of embodiments 216-220, wherein Y₄ is CO.
222. The compound of any one of embodiments 216-221, wherein both Y₃ and Y₄ are CO.
223. The compound of any one of embodiments 216-222, wherein Y₁ and Y₂ are 0, and Y₃ and Y₄ are CO.
224. The compound of any one of embodiments 216-223, wherein n₃ is 3.
225. The compound of any one of embodiments 216-224, wherein n₄ is 3.
226. The compound of any one of embodiments 216-225, wherein n₅ is 3.
227. The compound of any one of embodiments 216-226, wherein the oligonucleotide comprises an siRNA, an miRNA, an ADAR recruiting molecule, an ADAR targeting molecule, a guide RNA, or an antisense nucleic acid.
228. The compound of any one of embodiments 200-227, or salt, solvate, or hydrate thereof, for use as a reagent in a chemical reaction.
229. A composition comprising a compound of any one of embodiments 200-227, and a pharmaceutically acceptable carrier.
230. A method for modulating protein function in a subject, comprising administration of a compound of any one of embodiments 200-227, or a composition of embodiment 229, to the subject.
231. A method for treating or ameliorating a disease, disorder, or symptom thereof in a subject, comprising administration of a compound of any one of embodiments 200-227, or a composition of embodiment 229, to the subject.
232. The method of embodiment 231, wherein the disease, disorder, or symptom thereof is a liver disease, disorder, or symptom thereof.
233. A method for making a compound of any of embodiments 200-229 comprising one or more compounds and chemical transformations described herein, including Examples 1-23.

1. A compound of Formula (IX-a), or a salt, solvate, or hydrate thereof: 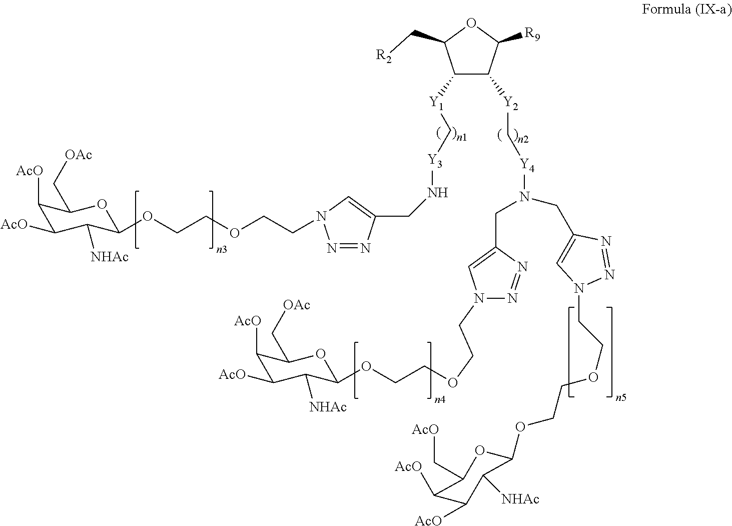
wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, optionally substituted heteroaryl, optionally substituted carbocyclyl, optionally substituted heterocyclyl, or a nucleobase isostere;
R² is H, OH, O-Protecting Group (PG), 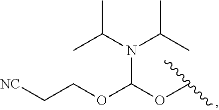 [SMILES: *OC(OCCC#N)N(C(C)C)C(C)C]
a linker, an azide, a carboxylic acid, or an amine;
Y₁ is O, optionally substituted NH, CH₂, or CH₂O;
Y₂ is O, optionally substituted NH, CH₂, or CH₂O;
Y₃ is CO, SO₂, P(O)O, CH₂—O—C(O), CH₂—NH—C(O), CH₂—NH—SO₂, or CH₂;
Y₄ is CO, SO₂, P(O)O, CH₂—O—C(O), CH₂—NH—C(O), CH₂—NH—SO₂, or CH₂;
n₂ is 0, 1, 2, 3, 4, 5, or 6; and
each of n₁, n₃, n₄, and n₅ is independently 0, 1, 2, 3, 4, 5, 6, 7, 8, 9, or 10.

2. The compound of claim 1, wherein R⁹ is uracil comprising a protecting group.

3-4. (canceled)

5. The compound of claim 1, wherein R² is

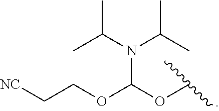 [SMILES: *OC(OCCC#N)N(C(C)C)C(C)C]

6. The compound of claim 1, wherein Y₁ is O.

7. The compound of claim 1, wherein Y₂ is O.

8. (canceled)

9. The compound of claim 1, wherein Y₃ is CO.

10. The compound of claim 1, wherein Y₄ is CO.

11-12. (canceled)

13. The compound of claim 1, wherein n₃, n₄, and n₅ are each 3.

14-16. (canceled)

17. The compound of claim 1, wherein the compound is of the formula: 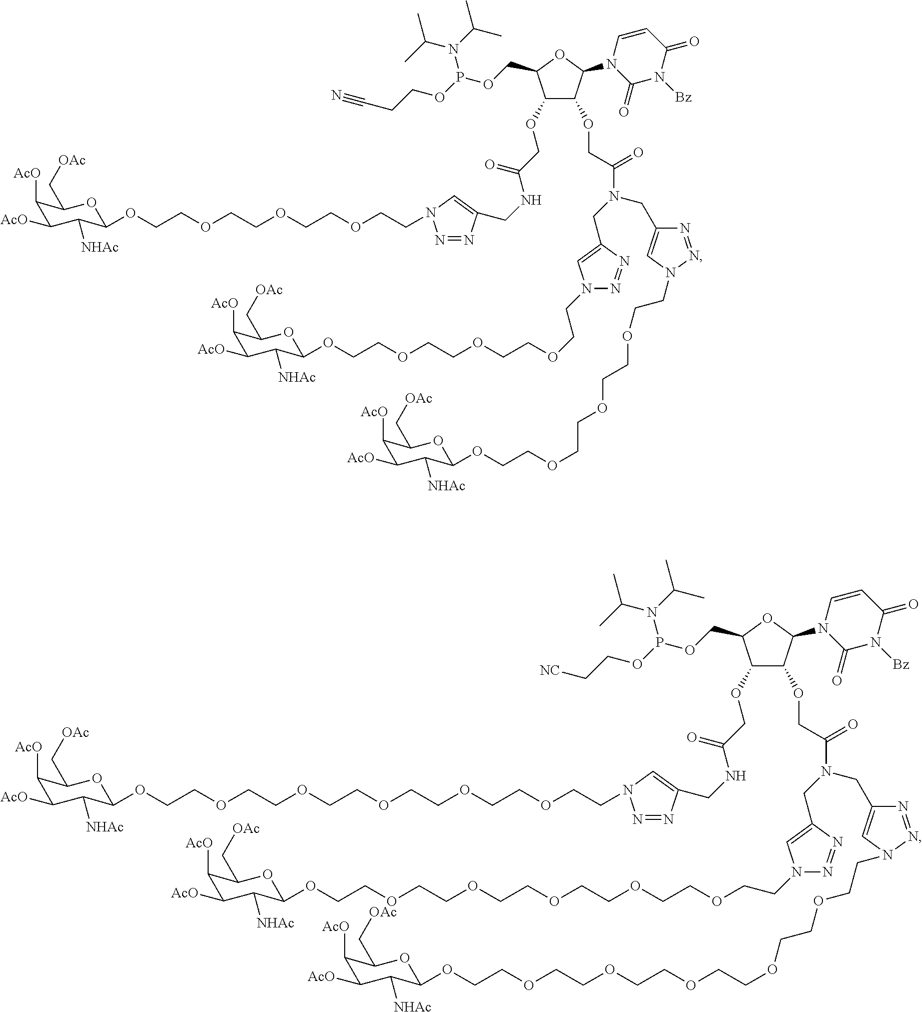 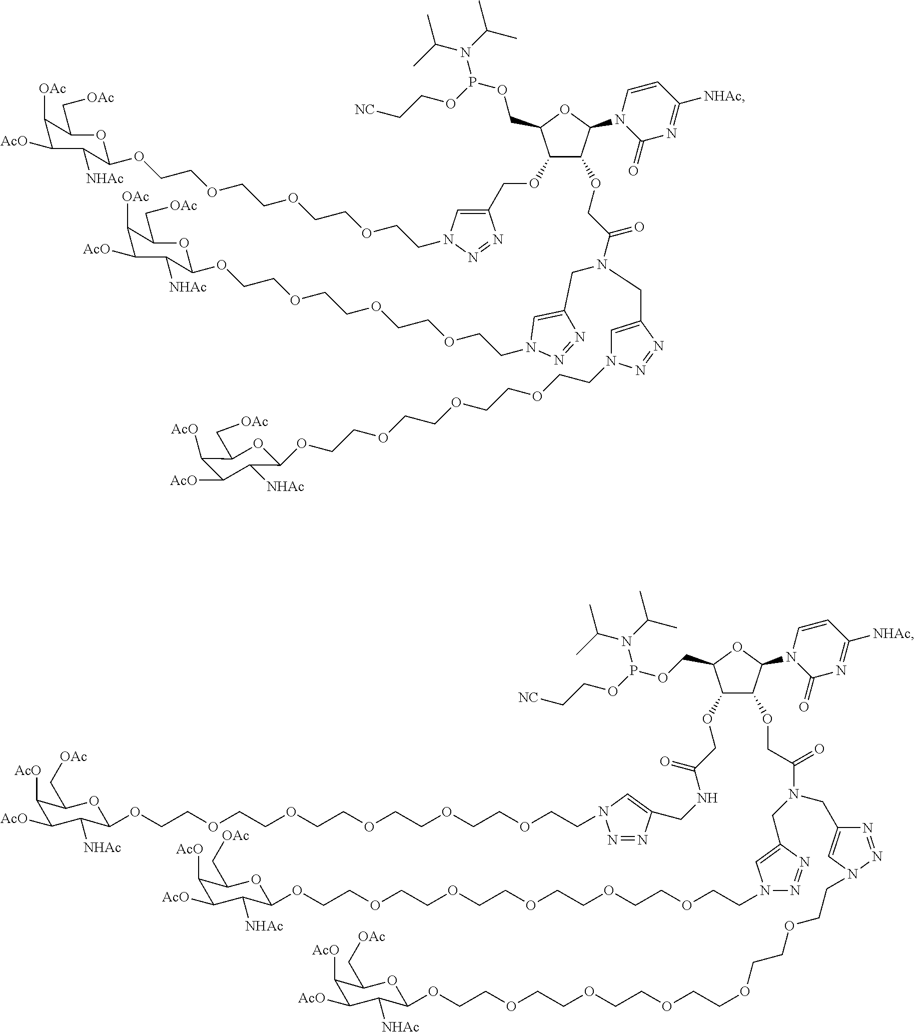 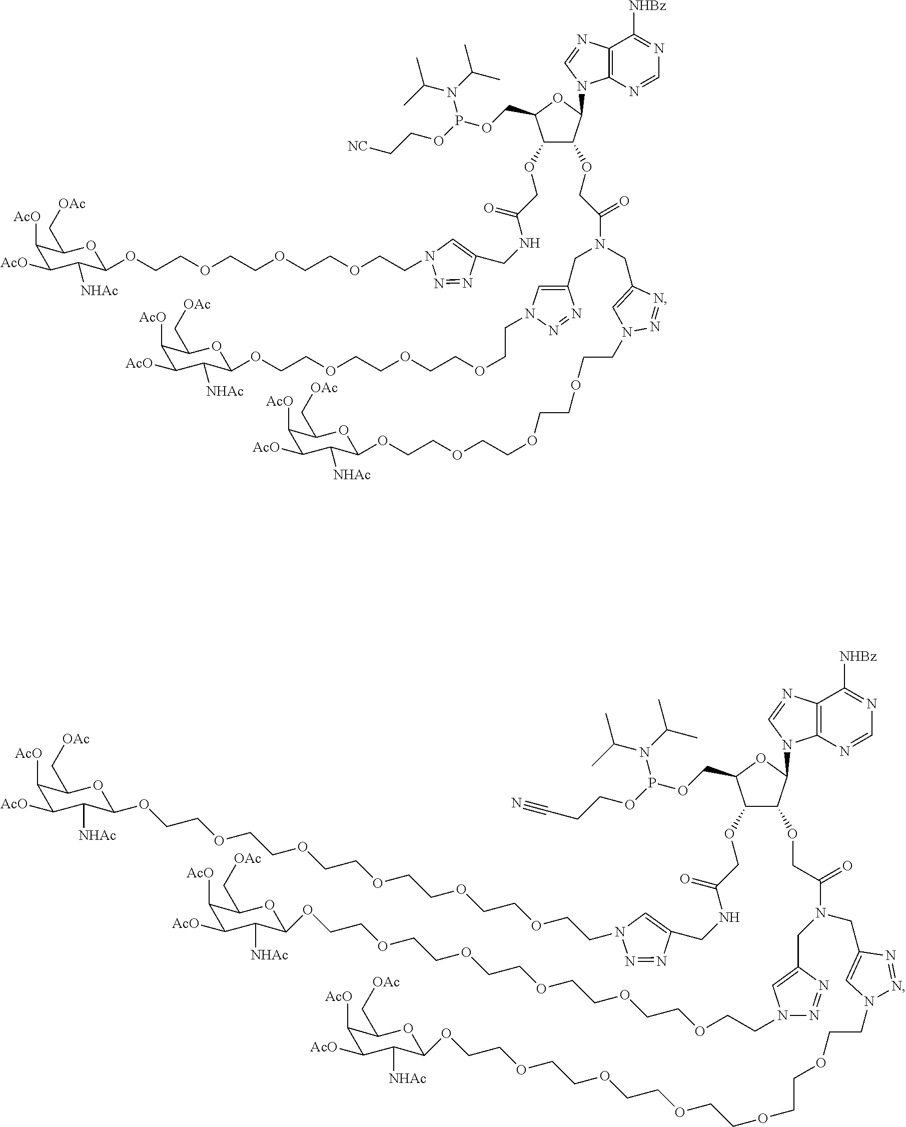 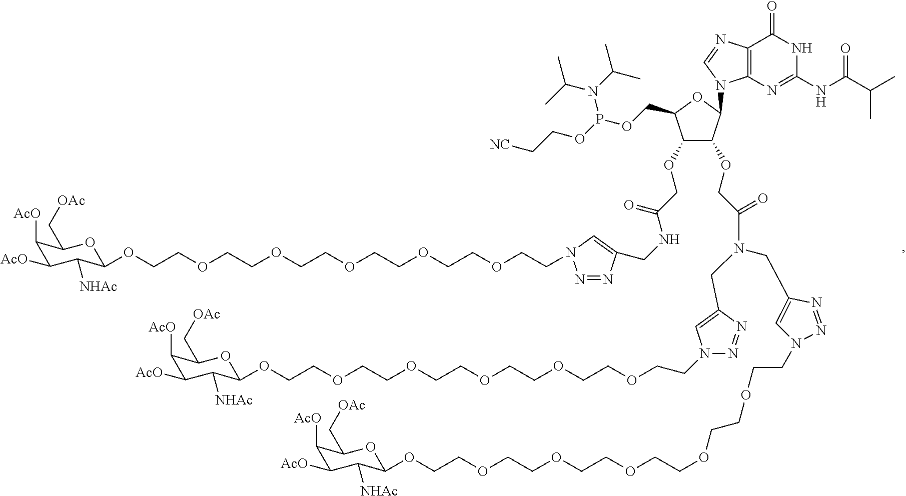 [SMILES: CC(=O)NC1C(OCCOCCOCCOCCOCCOCCn2cc(CNC(=O)CO[C@H]3[C@@H](OCC(=O)N(Cc4cn(CCOCCOCCOCCOCCOCCOC5OC(COC(C)=O)C(OC(C)=O)C(OC(C)=O)C5NC(C)=O)nn4)Cc4cn(CCOCCOCCOCCOCCOCCOC5OC(COC(C)=O)C(OC(C)=O)C(OC(C)=O)C5NC(C)=O)nn4)[C@H](n4cnc5c(=O)[nH]c(NC(=O)C(C)C)nc54)O[C@@H]3COP(OCCC#N)N(C(C)C)C(C)C)nn2)OC(COC(C)=O)C(OC(C)=O)C1OC(C)=O]
or a salt, solvate, or hydrate thereof.

18. (canceled)

19. A compound of Formula (XV-a), or a salt, solvate, or hydrate thereof: 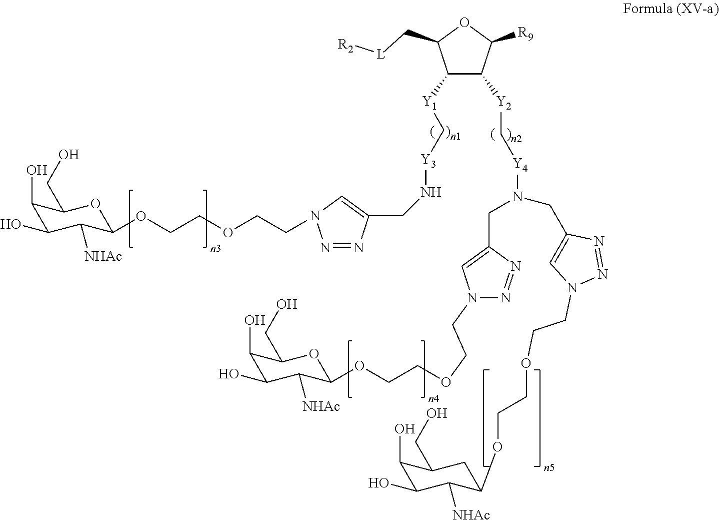
wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, optionally substituted heteroaryl, optionally substituted carbocyclyl, optionally substituted heterocyclyl, or a nucleobase isostere;
L is a bond, a phosphodiester bond, a phosphorothioate bond, a triazole, a tetrazole, an amide, a reverse-amide, a carbamate, a carbonate, urea, alkyl, or heteroalkyl;
R² is an oligonucleotide;
Y₁ is O, CH₂, CH₂O, or optionally substituted NH;
Y₂ is O, CH₂, CH₂O, or optionally substituted NH;
Y₃ is CO, SO₂, P(O)O, CH₂—O—C(O), CH₂—NH—C(O), CH₂—NH—SO₂, or CH₂;
Y₄ is CO, SO₂, P(O)O, CH₂—O—C(O), CH₂—NH—C(O), CH₂—NH—SO₂, or CH₂;
n₂ is 0, 1, 2, 3, 4, 5, or 6; and
each of n₁, n₃, n₄, and n₅ is independently 0, 1, 2, 3, 4, 5, 6, 7, 8, 9, or 10.

20. The compound of claim 19, wherein Y₁ is O.

21. The compound of claim 19, wherein Y₂ is O.

22. (canceled)

23. The compound of claim 19, wherein Y₃ is CO.

24. The compound of claim 19, wherein Y₄ is CO.

25-26. (canceled)

27. The compound of claim 19, wherein n₃, n₄, and n₅ are each 3.

28-29. (canceled)

30. The compound of claim 19, wherein the compound is of the formula: 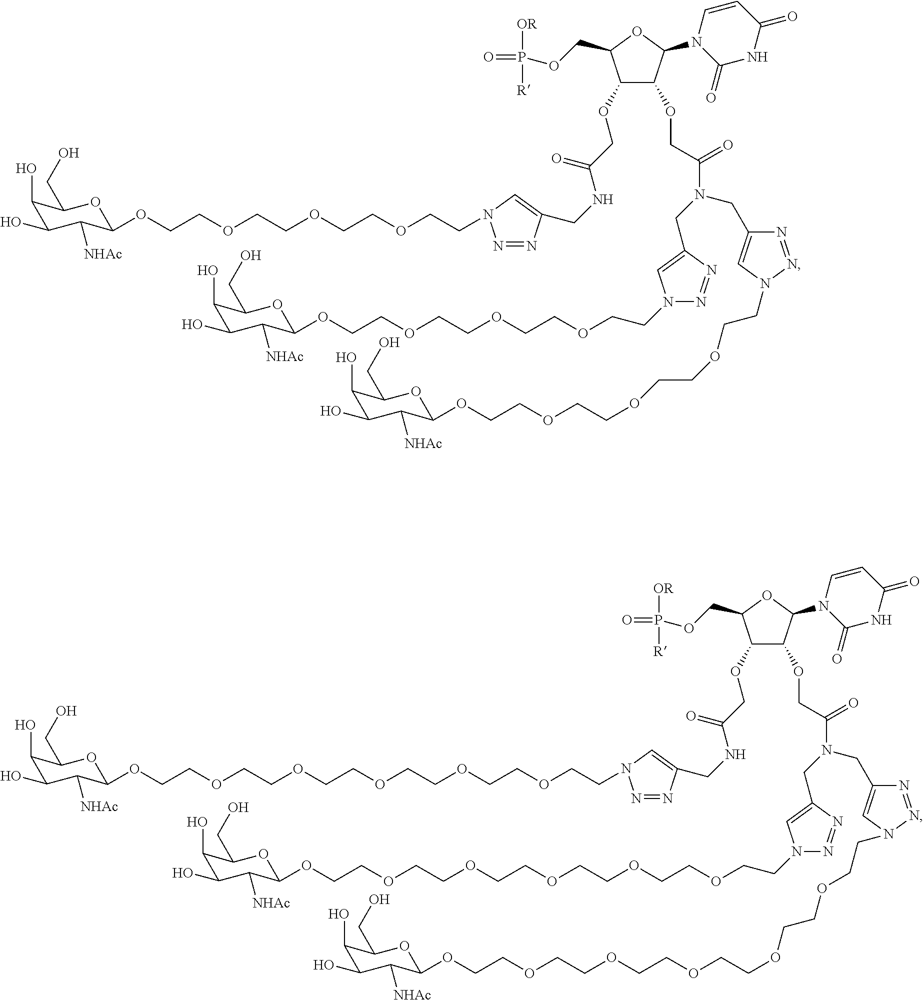 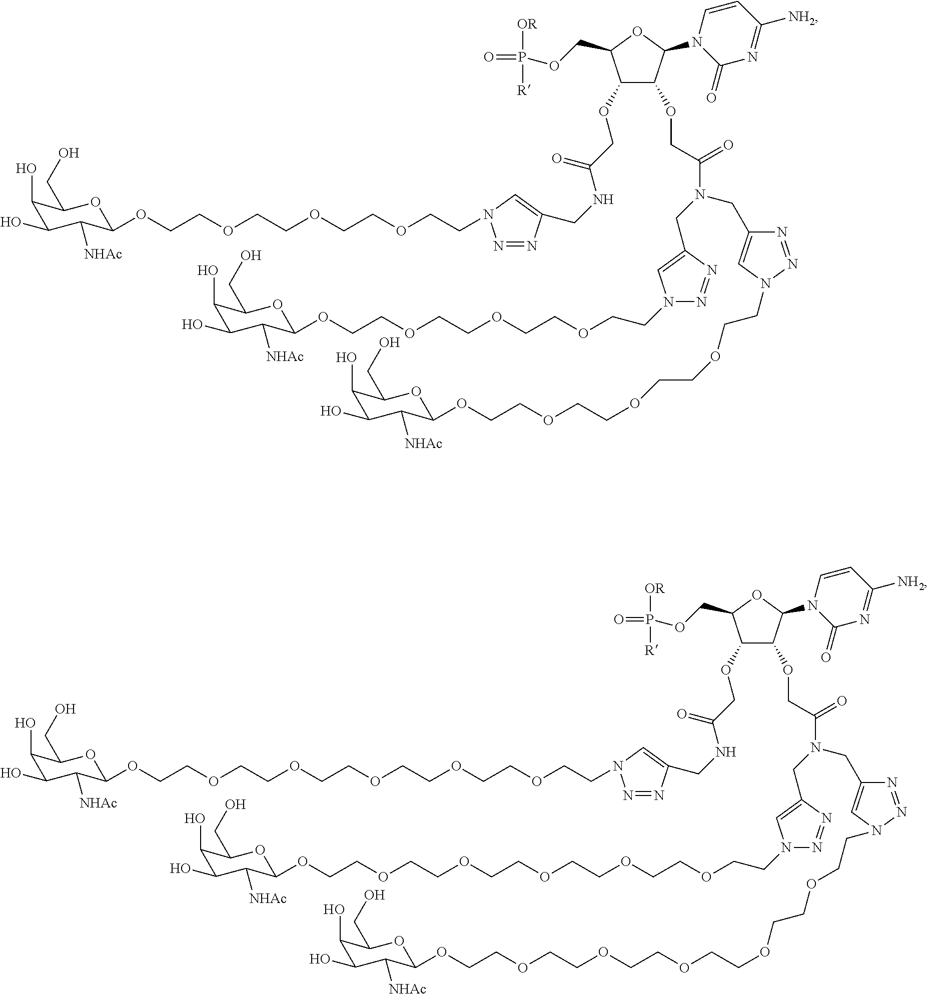 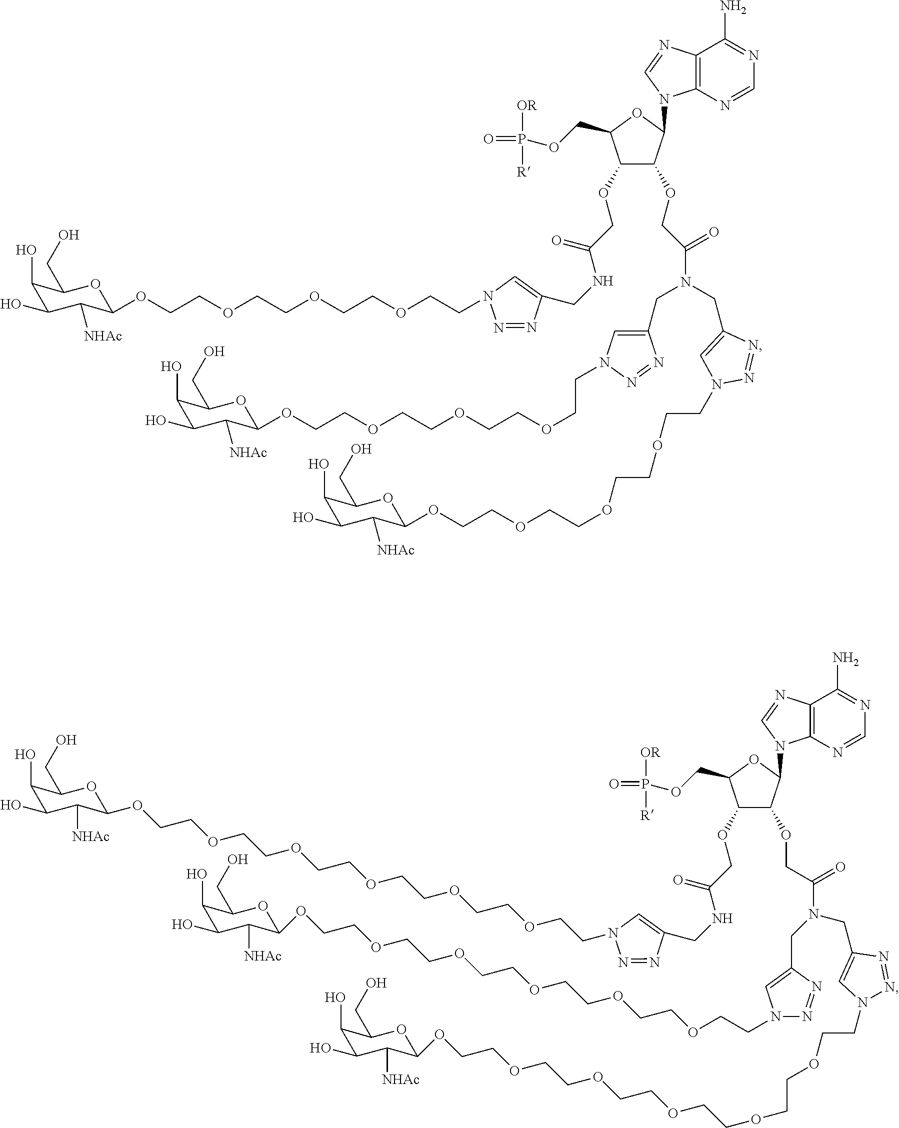 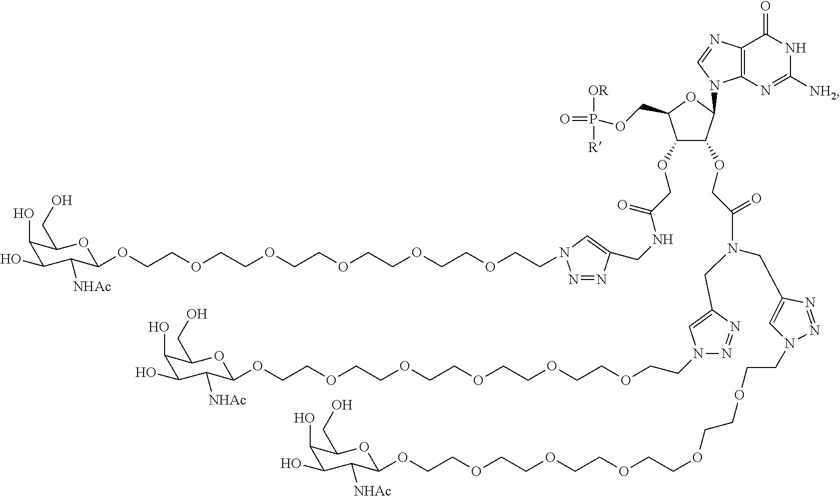
or a salt, solvate, or hydrate thereof;
wherein R is an oligonucleotide and R′ is O⁻ or S⁻.

31. (canceled)

32. The compound of claim 19, wherein the oligonucleotide comprises an siRNA, an miRNA, an ADAR recruiting molecule, an ADAR targeting molecule, a guide RNA, or an antisense nucleic acid.

33. (canceled)

34. A composition comprising a compound of claim 19, or a salt, solvate, or hydrate thereof, and a pharmaceutically acceptable carrier.

35. A method for modulating protein function in a subject, comprising administration of a compound of claim 19, or a salt, solvate, or hydrate thereof, to the subject.

36. A method for treating or ameliorating a disease, disorder, or symptom thereof in a subject, comprising administration of a compound of claim 19, or a salt, solvate, or hydrate thereof to the subject.

37. The method of claim 36, wherein the disease, disorder, or symptom thereof is a liver disease, disorder, or symptom thereof.

38. A method for making a compound of claim 1, or a salt, solvate, or hydrate thereof, comprising contacting a compound of the formula: 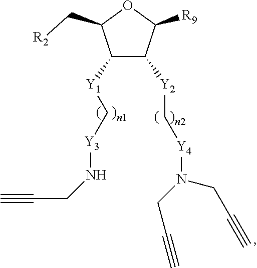
or a salt, solvate, or hydrate thereof, wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, optionally substituted heteroaryl, optionally substituted carbocyclyl, optionally substituted heterocyclyl, or a nucleobase isostere;
R² is H, OH, O-Protecting Group (PG), 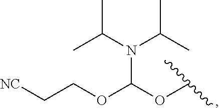 [SMILES: *OC(OCCC#N)N(C(C)C)C(C)C]
a linker, an azide, a carboxylic acid, or an amine;
Y₁ is O, optionally substituted NH, CH₂, or CH₂O;
Y₂ is O, optionally substituted NH, CH₂, or CH₂O;
Y₃ is CO, SO₂, P(O)O, CH₂—O—C(O), CH₂—NH—C(O), CH₂—NH—SO₂, or CH₂;
Y₄ is CO, SO₂, P(O)O, CH₂—O—C(O), CH₂—NH—C(O), CH₂—NH—SO₂, or CH₂;
n₁ is 0, 1, 2, 3, 4, 5, 6, 7, 8, 9, or 10; and
n₂ is 0, 1, 2, 3, 4, 5, or 6;
with compounds of the formulae: 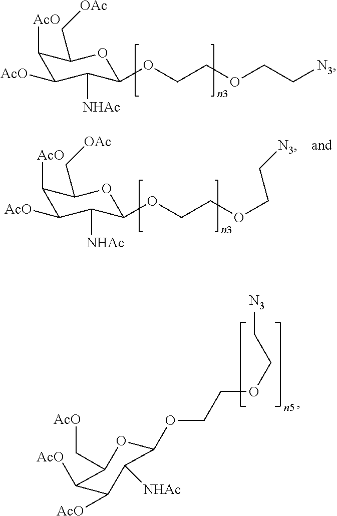
or salts, solvates, or hydrates thereof, wherein each of n₃, n₄, and n₅ is independently 0, 1, 2, 3, 4, 5, 6, 7, 8, 9, or 10.

39. A compound of Formula (I-a), (I-b), (I-c), or (I-d), or a salt, solvate, or hydrate thereof: 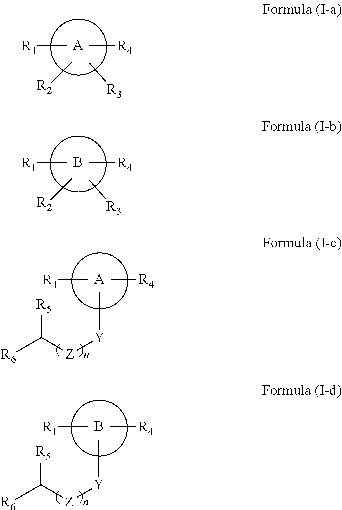
wherein: [symbol]
is Ring A, wherein each Ring A is independently optionally substituted carbocyclyl or optionally substituted heterocyclyl; [symbol]
is Ring B, wherein each Ring B is independently optionally substituted aryl or optionally substituted heteroaryl;
each n is independently 0, 1, 2, 3, or 4;
each Y is independently O, CH₂, S, S(═O), S(═O)₂, NH, substituted amino, NHC(═O), C(═O)NH, P(═O)₂—O—, P(═O)(═S)—O, P(═S)₂—O, —O—P(═O)₂—O—, —O—P(═O)(═S)—O—, —O—P(═S)₂—O—, —O—P(═O)₂—, —O—P(═O)(═S)—, —O—P(═S)₂—;
each Z is independently an optionally substituted alkyl group, an optionally substituted alkenyl group, an optionally substituted alkynyl group, or ethylene glycol;
each R₁ is independently an oligonucleotide, alkyl-O-phosphoramidite, alkyl phosphoramidite, optionally substituted alkenyl phosphoramidite, or optionally substituted alkynyl phosphoramidite;
R₂ and R₅ are each independently optionally substituted alkyl-O-GalNAc, polyethylene glycol-O-GalNAc, optionally substituted alkenyl-O-GalNAc, optionally substituted alkynyl-O-GalNAc, alkyl-S(═O)-alkyl-GalNAc, alkyl-S(═O)₂-alkyl-GalNAc, alkyl-S(═O)—NH-alkyl, alkyl-S(═O)₂—NH-alkyl-GalNAc, alkyl-P P(═O)(—O—)—NH-alkyl-GalNAc, alkyl-O—P(═O)(—O—)—O-alkyl-GalNAc, alkyl-O—P(—O—)(═S)—O-alkyl-GalNAc, or alkyl-O—P(—S—)(═S)—O-alkyl-GalNAc;
R₃ and R₆ are each independently optionally substituted alkyl-O-GalNAc, optionally substituted alkenyl-O-GalNAc, optionally substituted alkynyl-O-GalNAc, alkyl-S(═O)-alkyl-GalNAc, alkyl-S(═O)₂-alkyl-GalNAc, alkyl-S(═O)—NH-alkyl, alkyl-S(═O)₂—NH-alkyl-GalNAc, alkyl-P P(═O)(—O—)—NH-alkyl-GalNAc, alkyl-O—P(═O)(—O—)—O-alkyl-GalNAc, alkyl-O—P(—O—)(═S)—O-alkyl-GalNAc, or alkyl-O—P(—S—)(═S)—O-alkyl-GalNAc; and
each R₄ is independently optionally substituted alkyl-O-GalNAc, optionally substituted alkenyl-O-GalNAc, optionally substituted alkynyl-O-GalNAc, alkyl-S(═O)-alkyl-GalNAc, alkyl-S(═O)₂-alkyl-GalNAc, alkyl-S(═O)—NH-alkyl, alkyl-S(═O)₂—NH-alkyl-GalNAc, alkyl-P P(═O)(—O—)—NH-alkyl-GalNAc, alkyl-O—P(═O)(—O—)—O-alkyl-GalNAc, alkyl-O—P(—O—)(═S)—O-alkyl-GalNAc, or alkyl-O—P(—S—)(═S)—O-alkyl-GalNAc.

40. A compound of Formula (II-a), (II-b), or (II-c), or a salt, solvate, or hydrate thereof: 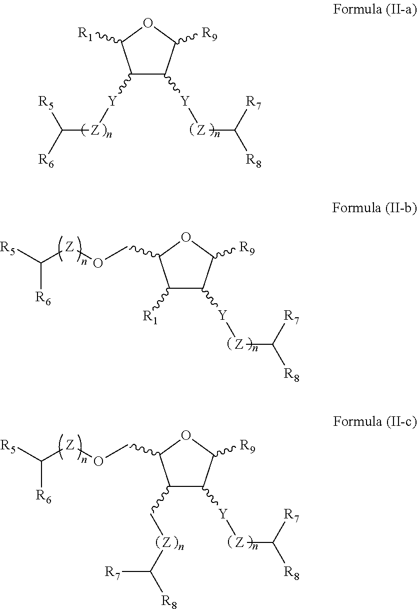
wherein:
each n is independently 0, 1, 2, 3, or 4;
each Y is independently O, CH₂, S, S(═O), S(═O)₂, NH, substituted N group, NHC(═O), C(═O)NH, P(═O)₂—O—, P(═O)(═S)—O, P(═S)₂—O, —O—P(═O)₂—O—, —O—P(═O)(═S)—O—, —O—P(═S)₂—O—, —O—P(═O)₂—, —O—P(═O)(═S)—, or —O—P(═S)₂—;
each Z is independently an optionally substituted alkyl group, an optionally substituted alkenyl group, an optionally substituted alkynyl group, or ethylene glycol;
each R₁ is independently an oligonucleotide, alkyl-O-phosphoramidite, optionally substituted alkenyl phosphoramidite, or optionally substituted alkynyl phosphoramidite;
R₅, R₆, R₇, and R₈ are each independently optionally substituted alkyl-O-GalNAc, polyethylene glycol-O-GalNAc, optionally substituted alkenyl-O-GalNAc, optionally substituted alkynyl-O-GalNAc, alkyl-S(═O)-alkyl-GalNAc, alkyl-S(═O)₂-alkyl-GalNAc, alkyl-S(═O)—NH-alkyl, alkyl-S(═O)₂—NH-alkyl-GalNAc, alkyl-P P(═O)(—O—)—NH-alkyl-GalNAc, alkyl-O—P(═O)(—O—)—O-alkyl-GalNAc, alkyl-O—P(—O—)(═S)—O-alkyl-GalNAc, or alkyl-O—P(—S—)(═S)—O-alkyl-GalNAc; and
R₉ is H, adenine, guanine, thymine, cytosine, uracil, inosine (I), or a nucleobase variant.

41. A compound of Formula (XXXI), or a salt, solvate, or hydrate thereof: 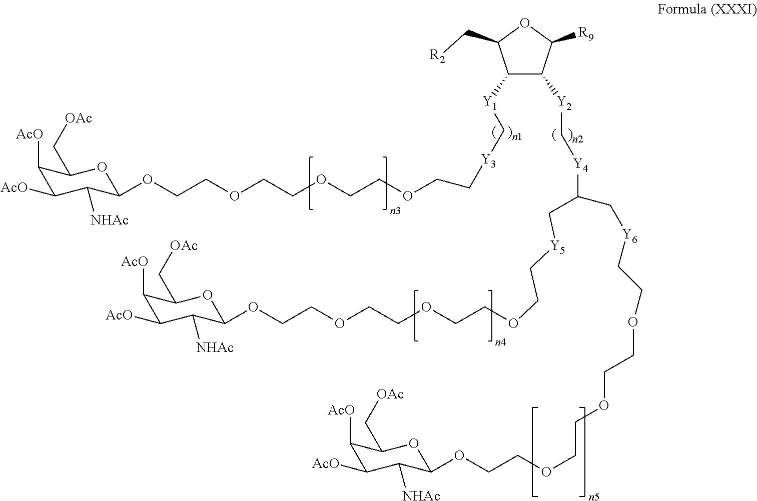
wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, optionally substituted heteroaryl, optionally substituted carbocyclyl, optionally substituted heterocyclyl, or a nucleobase isostere;
R² is H, O-Protecting Group (PG), 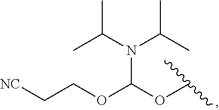 [SMILES: *OC(OCCC#N)N(C(C)C)C(C)C]
a linker, an azide, a carboxylic acid, an amine, or an oligonucleotide;
Y₁ is O, CH₂, CH₂O, or optionally substituted NH;
Y₂ is O, CH₂, CH₂O, or optionally substituted NH;
Y₃ is CO, C(O)NH, C(O)—NH—CH₂—C(O)—NH, SO₂, P(O)O, CH₂—O—C(O), CH₂—NH—C(O), CH₂—NH—SO₂, or CH₂;
Y₄ is CO, C(O)NH, SO₂, P(O)O, CH₂—O—C(O), CH₂—NH—C(O), CH₂—NH—SO₂, or CH₂;
Y₅ is CO, C(O)NH, OC(O)NH, or OCH₂C(O)NH;
Y₆ is CO, C(O)NH, OC(O)NH, or OCH₂C(O)NH;
n₂ is 0, 1, 2, 3, 4, 5, or 6;
each n₁, n₃, n₄, and n₅ is independently 0, 1, 2, 3, 4, 5, 6, 7, 8, 9, or 10.

42. A compound of Formula (XXXIII), or a salt, solvate, or hydrate thereof: 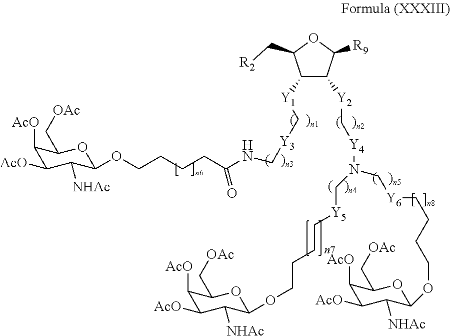
wherein:
R⁹ is H, adenine, guanine, thymine, cytosine, or uracil, or adenine, guanine, thymine, cytosine, or uracil, each comprising a Protecting Group (PG), a modified nucleobase, optionally substituted alkyl, optionally substituted aryl, optionally substituted heteroaryl, optionally substituted carbocyclyl, optionally substituted heterocyclyl, or a nucleobase isostere;
R² is H, O-Protecting Group (PG), 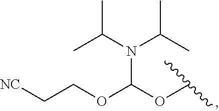 [SMILES: *OC(OCCC#N)N(C(C)C)C(C)C]
a linker, an azide, a carboxylic acid, or an amine;
Y₁ is O, CH₂, CH₂O, or optionally substituted NH;
Y₂ is O, CH₂, CH₂O, or optionally substituted NH;
Y₃ is CO, C(O)NH, C(O)—NH—CH₂—C(O)—NH, SO₂, P(O)O, CH₂—O—C(O), CH₂—NH—C(O), CH₂—NH—SO₂, or CH₂;
Y₄ is CO, C(O)NH, SO₂, P(O)O, CH₂—O—C(O), CH₂—NH—C(O), CH₂—NH—SO₂, or CH₂;
Y₅ is CO, C(O)NH, OC(O)NH, or OCH₂C(O)NH;
Y₆ is CO, C(O)NH, OC(O)NH, or OCH₂C(O)NH;
n₂ is 0, 1, 2, 3, 4, 5, or 6;
each n₁, n₃, n₄, n₅, n₆, n₇, and n₈ is independently 0, 1, 2, 3, 4, 5, 6, 7, 8, 9, or 10.
